(19) United States
(12) Patent Application Publication
(10) Pub. No.: US 2013/0029980 A1
(43) Pub. Date: Jan. 31, 2013

(54) FLAVIN DERIVATIVES

(71) Applicants: Philip D. G. Coish, North Haven, CT (US); Brian R. Dixon, Woodbridge, CT (US); David Osterman, Glastonbury, CT (US); Paul Adrian Aristoff, Dexter, MI (US); Manuel Navia, Boston, MA (US); Frank Sciavolino, Waterford, CT (US); Stephanie Avola, Woodbridge (CA); Nick Baboulas, Scarborough (CA); Thomas R. Belliotti, Portage, MI (US); Angelica Bello, North york (CA); Judd Berman, Toronto (CA); Robert A. Chrusciel, Portage, MI (US); Bruce R. Evans, Holland, MI (US); Harpreet Kaur, Brampton (CA); David Moon, Brampton (CA); Vinh Pham, Markham (CA); Andrew Roughton, Port Hope (CA); Phil Wickens, Richmond Hill (CA); Jeffrey Wilson, Toronto (CA); Heinrich J. Schostarez, Portage, MI (US)

(72) Inventors: Philip D. G. Coish, North Haven, CT (US); Brian R. Dixon, Woodbridge, CT (US); David Osterman, Glastonbury, CT (US); Paul Adrian Aristoff, Dexter, MI (US); Manuel Navia, Boston, MA (US); Frank Sciavolino, Waterford, CT (US); Stephanie Avola, Woodbridge (CA); Nick Baboulas, Scarborough (CA); Thomas R. Belliotti, Portage, MI (US); Angelica Bello, North york (CA); Judd Berman, Toronto (CA); Robert A. Chrusciel, Portage, MI (US); Bruce R. Evans, Holland, MI (US); Harpreet Kaur, Brampton (CA); David Moon, Brampton (CA); Vinh Pham, Markham (CA); Andrew Roughton, Port Hope (CA); Phil Wickens, Richmond Hill (CA); Jeffrey Wilson, Toronto (CA); Heinrich J. Schostarez, Portage, MI (US)

(21) Appl. No.: 13/640,054

(22) PCT Filed: Apr. 6, 2011
(86) PCT No.: PCT/US11/00617

§ 371 Date: Oct. 9, 2012

Related U.S. Application Data
(60) Provisional application No. 61/321,347, filed on Apr. 6, 2010.
(60) Provisional application No. 61/441,160, filed on Feb. 9, 2011.

(51) Int. Cl. C07D 487/04; A61P 31/04; A61K 31/5377; A61K 31/519; C07D 491/147

(52) U.S. Cl. 514/232.8; 544/251; 514/250; 544/247; 514/249; 544/345; 544/115; 540/477

(57) ABSTRACT

The present invention relates novel flavin derivatives, their use and compositions for use as riboswitch ligands and/or anti-infectives.

### TECHNICAL FIELD

The present invention relates to flavin derivatives and their use and compositions for use as riboswitch ligands and/or anti-infectives.

### BACKGROUND OF THE INVENTION

The fast growing rate of antibiotic resistance over the past decades has raised serious concerns that the antibiotic treatment options currently available will soon be ineffective. With the widespread usage of antibiotics in combination with the rapid growing rate of bacterial resistance in stark contrast with the decade-old chemical scaffolds available for their treatment, it is imperative that new drugs are developed in the battle against bacterial pathogens.

In many bacteria and fungi, RNA structures termed riboswitches regulate the expression of various genes crucial for survival or virulence. Typically located within the 5′-untranslated region (5′-UTR) of certain mRNAs, members of each known class of riboswitch can fold into a distinct, three-dimensionally structured receptor that recognizes a specific organic metabolite. When the cognate metabolite is present at sufficiently high concentrations during transcription of the mRNA, the riboswitch receptor binds to the metabolite and induces a structural change in the nascent mRNA that prevents expression of the open reading frame (ORF), thereby altering gene expression. In the absence of the cognate metabolite, the riboswitch folds into a structure that does not interfere with the expression of the ORF.

Sixteen different classes of riboswitches have been reported. Members of each class of riboswitch bind to the same metabolite and share a highly conserved sequence and secondary structure. Riboswitch motifs have been identified that bind to thiamine pyrophosphate (TPP), flavin mononucleotide (FMN), glycine, guanine, 3′-5′-cyclic eiguanylic acid (c-di-GMP), molybdenum cofactor, glucosamine-6-phosphate (GlcN6P), lysine, adenine, and adocobalamin (AdoCbl) riboswitches. Additionally, four dinstinct riboswitch motifs have been identified that recognize S-adenosylmethionine (SAM) I, II and III, IV and two distinct motifs that recognize pre-queosine-1 (PreQ1). Several antimetabolite ligands have also been identified that bind to known riboswitch classes, including pyrithiamine pyrophosphate (PTPP) which binds TPP riboswitches, L-aminoethylcysteine (AEC) and DL-4-oxalysine which bind to lysine riboswitches and roseoflavin and FMN which bind to FMN riboswitches. The riboswitch-receptors bind to their respective ligands in an interface that approaches the level of complexity and selectivity of proteins. This highly specific interaction allows riboswitches to discriminate against most intimately related analogs of ligands. For instance, the receptor of a guanine-binding riboswitch from Bacillus subtilis forms a three-dimensional structure such that the ligand is almost completely enveloped. The guanine is positioned between two aromatic bases and each polar functional group of the guanine hydrogen bonds with four additional riboswitch nucleotides surrounding it. This level of specificity allows the riboswitch to discriminate against most closely related purine analogs. Similarly, studies of the SAM-binding riboswitches reveal that nearly every functional group of SAM is critical in binding the ligands, allowing it to discriminate highly similar compounds such as S-adenosylhomocysteine (SAH) and S-adenosylmethionine (SAM), which only differ by a single methyl group. Likewise, TPP riboswitches comprise one subdomain that recognizes every polar functional group of the 4-amino-5-hydroxymethyl-2-methylpyrimidine (HMP) moiety, albeit not the thiazole moiety, and another subdomain that coordinates two metal ions and several water molecules to bind the negatively charged pyrophosphate moiety of the ligand. Similar to TPP, guanine and SAM riboswitches, FMN riboswitches form receptor structures that are highly specific for the natural metabolite FMN. It is by this highly specific interaction that allows for the design of small molecules for the regulation of specific genes.

FMN riboswitches are of particular interest of this invention because it is believed that the riboswitch binds to flavin mono-nucleotide (FMN) and represses the expression of enzymes responsible for riboflavin and FMN biosynthesis. Riboflavin is a water-soluble vitamin that is converted by flavokinases and FAD synthases to co-factors FMN and FAD, which are indispensable cofactors involved in energy metabolism and metabolism of fats, ketones, carbohydrates and proteins crucial for all living organisms. Although vertebrates rely on uptake of vitamin from their gut for riboflavin sources, most prokaryotes, fungi and plants synthesize the necessary riboflavin for survival. It is therefore suggested that compounds that are selective for FMN riboswitch may be useful targets against bacterial pathogens in shutting down biosynthesis of riboflavin crucial for survival or virulence. In addition, no examples of the FMN, TPP, nor any other riboswitch class have presently been identified in humans. Therefore, riboswitches appear to offer the potential for the discovery of selective antipathogenic drugs. It is therefore the objective of this invention to provide novel flavin derivatives for targeting FMN riboswitch and methods of treating infections comprising administering flavin derivatives. Flavin derivatives that target FMN riboswitch are generically disclosed in PCT/US2009/004576 and PCT/US2010/001876, the contents of which are incorporated by reference in their entirety. The current application provides further flavin derivatives that target the FMN and/or the CD3299 riboswitch and/or are active against various bacterial strains.

### SUMMARY OF THE INVENTION

The invention relates to a compound of Formula P:

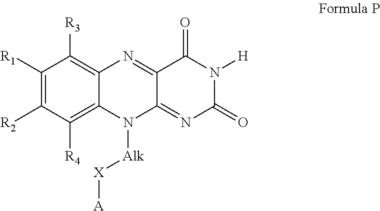

wherein:
(i) Alk is C₁₋₆alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl, ethyl or isobutyl), arylC₁₋₄alkyl (e.g., benzyl) and/or —N(R_{c})(R_{d}); or
   Alk is C₁₋₆alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄alkoxy (e.g., methoxy, ethoxy, propoxy, isobutoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
   C₁₋₄alkyl (e.g., methyl, ethyl, t-butyl or n-prop-2-en-1-yl),
   C₁₋₄alkoxy (e.g., methoxy),
   hydroxy,
   —O—C₁₋₄alkyl-N(R_{c})(R_{d}), for example —OCH₂CH₂N(CH₃)₂,
   halo (e.g., Cl, F),
   haloC₁₋₄alkyl (e.g., CF₃),
   —O-haloC₁₋₄alkyl (e.g., —OCF₃),
   cyano,
   —O—(CH₂CH₂O)₁₋₃—C₁₋₄alkyl (e.g., —OCH₂CH₂OCH₃ or —O(CH₂CH₂O)₃CH₃), and/or
   —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
   H,
   C₁₋₆alkyl, e.g., C₁₋₄alkyl (for example, methyl, ethyl, n-propyl, isopropyl, isobutyl, t-butyl, 1-methylpropyl or n-hexyl),
   C₃₋₈cycloalkyl (e.g., cyclopropyl or cyclopentyl),
   aryl (e.g., phenyl), or
   C₁₋₄alkoxy (e.g., methoxy);
(v) R₂ is:
   H,
   C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
   —C₀₋₄alkyl-C₃₋₈cycloalkyl (e.g., cyclopropyl),
   —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
   —C₀₋₄alkyl-N(Rₐ)(R_{b}), for example —C₀alkyl-N(Rₐ)(R_{b}) or —C₁alkyl-N(Rₐ)(R_{b}),
   C₁₋₄alkoxy (e.g., methoxy),
   halo (e.g., Cl),
   —O—(CH₂CH₂O)₁₋₃—C₁₋₄alkyl (e.g., —OCH₂CH₂OCH₃ or —O(CH₂CH₂O)₃CH₃),
   —N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
   —N(Rₑ)—C(O)—O—C₁₋₄alkyl (e.g., —N(H)—C(O)—O—C(H)(CH₃)CH₃),
   —N(Rₑ)—C(O)-aryl wherein said aryl is optionally substituted with one or more halo (e.g., F), for example —N(H)—C(O)-(4-fluorophenyl),
   —C₁₋₆alkyl-OC₁₋₄alkyl (e.g., —CH₂CH₂CH₂CH₂—O—CH₃),
   —O—CH₂CH₂—O—CH₂-phenyl,
   —O-haloC₁₋₄alkyl (e.g., —OCH₂CF₃),
   —CH₂—O—C(O)—C₁₋₄alkyl (e.g., —CH₂—O—C(O)—CH₃),
   —C(O)O—C₁₋₄alkyl (e.g., —C(O)OCH₃), or
   C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
   H,
   C₁₋₄alkyl (e.g., methyl) optionally substituted with one or more hydroxy groups for example, 2,3-dihydroxyprop-1-yl,
   C₃₋₈cycloalkyl (e.g., cyclopropyl or cyclopentyl),
   C₁₋₄alkoxy-C₁₋₄alkyl (e.g., methoxyethyl),
   hydroxy-C₁₋₄alkyl (e.g., hydroxyethyl),
   N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
   in free or salt form.

The invention relates to a compound of Formula Q:

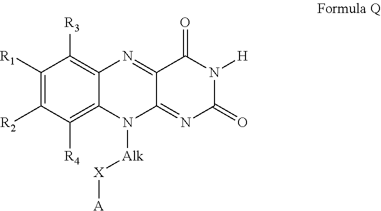

wherein:
(i) Alk is C₁₋₆alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl or isobutyl) and/or —N(R_{c})(R_{d}); or
   Alk is C₁₋₆alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄-alkoxy (e.g., methoxy, ethoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
   C₁₋₄alkyl (e.g., methyl, t-butyl or n-prop-2-en-1-yl),
   C₁₋₄alkoxy (e.g., methoxy),
   hydroxy,
   —O—C₁₋₄alkyl-N(R_{c})(R_{d}), for example —OCH₂CH₂N(CH₃)₂,
   halo (e.g., Cl, F),
   haloC₁₋₄alkyl (e.g., CF₃),
   —O-haloC₁₋₄alkyl (e.g., —OCF₃),
   cyano,
   —O—(CH₂CH₂O)₁₋₃—C₁₋₄alkyl (e.g., —OCH₂CH₂OCH₃ or —O(CH₂CH₂O)₃CH₃), and/or
   —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
   H,
   C₁₋₆alkyl, e.g., C₁₋₄alkyl (for example, methyl, ethyl, n-propyl, isopropyl, isobutyl, t-butyl, 1-methylpropyl or n-hexyl),
   C₃₋₈cycloalkyl (e.g., cyclopropyl),
   aryl (e.g., phenyl), or
   C₁₋₄alkoxy (e.g., methoxy);
(v) R₂ is:
   H,
   C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
   —C₀₋₄alkyl-C₃₋₈cycloalkyl (e.g., cyclopropyl),
   —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
   —C₀₋₄alkyl-N(Rₐ)(R_{b}), for example —C₀alkyl-N(Rₐ)(R_{b}) or —C₁alkyl-N(Rₐ)(R_{b}),
   C₁₋₄alkoxy (e.g., methoxy),
   halo (e.g., Cl),
   —O—(CH₂CH₂O)₁₋₃—C₁₋₄alkyl (e.g., —OCH₂CH₂OCH₃ or —O(CH₂CH₂O)₃CH₃),
   —N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
   —N(Rₑ)—C(O)—O—C₁₋₄alkyl (e.g., —N(H)—C(O)—O—C(H)(CH₃)CH₃),
   —N(Rₑ)—C(O)-aryl wherein said aryl is optionally substituted with one or more halo (e.g., F), for example —N(H)—C(O)-(4-fluorophenyl),
   —C₁₋₆alkyl-OC₁₋₄alkyl (e.g., —CH₂CH₂CH₂CH₂—O—CH₃),
   —O—CH₂CH₂—O—CH₂-phenyl,
   —O-haloC₁₋₄alkyl (e.g., —OCH₂CF₃),
   —CH₂—O—C(O)—C₁₋₄alkyl (e.g., —CH₂—O—C(O)—CH₃),
   —C(O)O—C₁₋₄alkyl (e.g., —C(O)OCH₃), or
   C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
   H,
   C₁₋₄alkyl (e.g., methyl) optionally substituted with one or more hydroxy groups for example, 2,3-dihydroxyprop-1-yl,
   C₃₋₈cycloalkyl (e.g., cyclopropyl or cyclopentyl),
   C₁₋₄ alkoxy-C₁₋₄alkyl (e.g., methoxyethyl),
   hydroxy-C₁₋₄ alkyl (e.g., hydroxyethyl),
   N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
   in free or salt form.

In a further embodiment, the invention relates to a compound of Formula I:

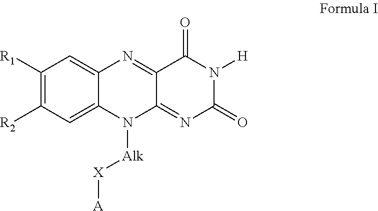

wherein:
(i) Alk is C₁₋₄alkylene (e.g., ethylene, n-propylene, n-butylene, n-pentylene) optionally substituted with one or more C₁₋₄alkyl, —N(R_{c})(R_{d}); or
   Alk is C₁₋₆alkylene (e.g., n-propylene, n-butylene, n-pentylene) optionally substituted with one hydroxy or C₁₋₄alkoxy group;
(ii) X is a single bond, —S— or —O—;
(iii) A is aryl (e.g., phenyl) or aryl-C₁₋₄alkyl (e.g., benzyl), wherein the aryl group of said aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl) or C₁₋₄alkoxy (e.g., methoxy);
(v) R₂ is H, C₁₋₄alkyl (e.g., methyl), —C₀₋₄alkyl-C₃₋₈cycloalkyl (e.g., cyclopropyl), —C₁₋₄alkyl-N(Rₐ)(R_{b}), C₁₋₄alkoxy (e.g., methoxy), halo (e.g., Cl), or C₃₋₈heterocycloalkyl (e.g., pyrrolidinyl, for example pyrrolidin-1-yl)
   wherein said heterocycloalkyl is optionally substituted with one or more hydroxy; or
(vi) Optionally, R₁ and R₂ are linked together so that together with the carbon atoms to which they are attached they form a cyclic structure (e.g., R₁ and R₂ are linked together to form ethylenedioxy);
(vii) Rₐ and R_{b} are independently H, C₁₋₄alkyl (e.g., methyl), C₃₋₈cycloalkyl (e.g., cyclopropyl, cyclopentyl), C₁₋₄alkoxy-C₁₋₄alkyl (e.g., methoxyethyl), hydroxy-C₁₋₄alkyl (e.g., hydroxyethyl), N(R_{c})(R_{d})—C₁₋₄alkyl (e.g., dimethylaminoethyl);
(viii) R_{c} and R_{d} are independently H or C₁₋₄alkyl (e.g., methyl);
   in free or salt form.

The invention further relates to a compound of Formula P as described in the following formulae:
P.1. The compound of Formula P wherein;
   (i) Alk is C₁₋₆alkylene (e.g., C₂₋₅alkylene, for example ethylene, i.e., CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl, ethyl or isobutyl), arylC₁₋₄alkyl (e.g., benzyl) and/or —N(R_{c})(R_{d}); or
      Alk is C₁₋₆alkylene (e.g., C₂₋₅alkylene, for example n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one hydroxy or C₁₋₄alkoxy (e.g., methoxy, ethoxy, propoxy, isobutoxy or isopropyloxy) group;
   (ii) X is a single bond, —S—, —S(O)₂—, —S(O)— or —O—;
   (iii) A is aryl (e.g., phenyl or naphthyl) or aryl-C₁₋₄ alkyl (e.g., benzyl or naphthylmethyl), wherein the aryl group of said aryl or arylalkyl is optionally substituted with one or more
      C₁₋₄alkyl (e.g., methyl, ethyl, t-butyl or n-prop-2-en-1-yl),
      C₁₋₄alkoxy (e.g., methoxy),
      hydroxy,
      —O—C₁₋₄alkyl-N(R_{c})(R_{d}), for example —
      OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      —O—(CH₂CH₂O)₁₋₃—C₁₋₄alkyl (e.g., —OCH₂CH₂OCH₃ or —O(CH₂CH₂O)₃CH₃), and/or
      —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl);
   (iv) R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
      C₃₋₈cycloalkyl (e.g., cyclopropyl or cyclopentyl), aryl (e.g., phenyl), or
      C₁₋₄alkoxy (e.g., methoxy);
   (v) R₂ is:
      H,
      C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
      —C₀₋₄alkyl-C₃₋₈cycloalkyl (e.g., cyclopropyl),
      —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
      —C₀₋₄alkyl-N(Rₐ)(R_{b}), for example —C₀alkyl-N(Rₐ)(R_{b}) or
      —C₁alkyl-N(Rₐ)(R_{b}),
      C₁₋₄alkoxy (e.g., methoxy),
      halo (e.g., Cl),
      —O—(CH₂CH₂O)₁₋₃—C₁₋₄alkyl (e.g., —OCH₂CH₂OCH₃ or —O(CH₂CH₂O)₃CH₃),
      —N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
      —N(Rₑ)—C(O)—O—C₁₋₄alkyl (e.g., —N(H)—C(O)—C(H)(CH₃)CH₃),
      —N(Rₑ)—C(O)-aryl wherein said aryl is optionally substituted with one or more halo (e.g., F), for example —N(H)—C(O)-(4-fluorophenyl),
      —C₁₋₆alkyl-OC₁₋₄alkyl (e.g., —CH₂CH₂CH₂CH₂—O—CH₃),
      —O—CH₂CH₂—O—CH₂-phenyl,
      —O-haloC₁₋₄alkyl (e.g., —OCH₂CF₃),
      —CH₂—O—C(O)—C₁₋₄alkyl (e.g., —CH₂—O—C(O)—CH₃),
      —C(O)O—C₁₋₄alkyl (e.g., —C(O)OCH₃), or
      C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
   (vi) Optionally, R₁ and R₂ are linked together so that together with the carbon atoms to which they are attached they form a cyclic structure (e.g., R₁ and R₂ are linked together to form ethylenedioxy);
   (vii) Rₐ and R_{b} are independently:
      H,
      C₁₋₄alkyl (e.g., methyl) optionally substituted with one or more hydroxy groups for example, 2,3-dihydroxyprop-1-yl,
      C₃₋₈cycloalkyl (e.g., cyclopropyl or cyclopentyl),
      C₁₋₄alkoxy-C₁₋₄alkyl (e.g., methoxyethyl),
      hydroxy-C₁₋₄alkyl (e.g., hydroxyethyl),
      N(R_{c})(R_{d})—C₁₋₄alkyl (e.g., dimethylaminoethyl);
   (viii) R_{c} and R_{d} are independently H, C₁₋₄alkyl (e.g., methyl) or arylC₁₋₄alkyl (e.g., benzyl);
   (ix) R₃ and R₄ are independently H or C₁₋₄alkyl (e.g., methyl);
   (x) Rₑ is H or C₁₋₄alkyl;
P.2. the compound of formula P or P.1, wherein R₂ is:
   H,
   C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
   —C₀₋₄alkyl-C₃₋₈cycloalkyl (e.g., cyclopropyl),
   —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
   —C₀₋₁alkyl-N(Rₐ)(R_{b}), wherein Rₐ is H and R_{b} is C₁₋₄alkoxy-C₁₋₄alkyl (e.g., methoxyethyl) or both Rₐ and R_{b} are methyl,
   C₁₋₄alkoxy (e.g., methoxy),
   halo (e.g., Cl),
   —C₁₋₆alkyl-OC₁₋₄alkyl (e.g., —CH₂CH₂CH₂CH₂—O—CH₃),
   —O-haloC₁₋₄alkyl (e.g., —OCH₂CF₃),
   —CH₂—O—C(O)—C₁₋₄alkyl (e.g., —CH₂—O—C(O)—CH₃),
   —C(O)O—C₁₋₄alkyl (e.g., —C(O)OCH₃), or
   C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
P.3. the compound of formula P, P.1 or P.2, wherein:
   (i) Alk is C₁₋₆alkylene (e.g., C₂₋₅alkylene, for example ethylene, i.e., —CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl, ethyl or isobutyl), arylC₁₋₄alkyl (e.g., benzyl); or
      Alk is C₁₋₆alkylene (e.g., C₂₋₅alkylene, for example n-propylene, i.e., —CH₂CH₂CH₂—, n-butylene, i.e., —CH₂CH₂CH₂CH₂—, n-pentylene, i.e., —CH₂CH₂CH₂CH₂CH₂—) optionally substituted with one hydroxy or C₁₋₄alkoxy (e.g., methoxy, ethoxy, propoxy, isobutoxy or isopropyloxy) group;
P.4. the compound of formula P or any of P.1-P.3 wherein:
   A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, ethyl, t-butyl or n-prop-2-en-1-yl),
      C₁₋₄alkoxy (e.g., methoxy),
      hydroxy,
      —O—C₁₋₄alkyl-N(R_{c})(R_{d}), for example —OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      —OCH₂CH₂OCH₃, and/or
      —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl);
P.5. the compound of formula P wherein:
   (i) Alk is C₁₋₄alkylene (e.g., C₂₋₅alkylene, for example ethylene, i.e., —CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl, ethyl or isobutyl), arylC₁₋₄alkyl (e.g., benzyl) and/or —N(R_{c})(R_{d}); or
      Alk is C₁₋₆alkylene (e.g., C₂₋₅alkylene, for example n-propylene, i.e., —CH₂CH₂CH₂—, n-butylene, i.e., —CH₂CH₂CH₂CH₂—, n-pentylene, i.e., —CH₂CH₂CH₂CH₂CH₂—) optionally substituted with one hydroxy or C₁₋₄alkoxy (e.g., methoxy, ethoxy, isobutoxy or isopropyloxy) group; and
   (ii) X is a single bond, —S— or —O—;
   (iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, ethyl t-butyl),
      —O—C₁₋₄alkyl-N(R_{c})(R_{d}), for example —OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      [2,6-dimethylmorpholin-4-yl]methyl, e.g. [(2R,6S)-2,6-dimethylmorpholin-4-yl]methyl);
   (iv) R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
      C₃₋₈cycloalkyl (e.g., cyclopropyl or cyclopentyl),
      aryl (e.g., phenyl), or
      C₁₋₄alkoxy (e.g., methoxy);
   (v) R₂ is:
      H,
      C₁₋₆alkyl, e.g., C₁₋₄alkyl (for example, methyl, ethyl, n-propyl, isopropyl, n-butyl, isobutyl, n-but-2-en-1-yl, n-hexyl),
      —C₀₋₄alkyl-C₃₋₈cycloalkyl (e.g., cyclopropyl), —C₁₋₄alkyl-heteroC₃₋₈cycloalkyl, wherein said heterocycloalkyl is optionally substituted with one or more C₁₋₄alkyl (e.g., methyl) groups, for example, [2,6-dimethylmorpholin-4-yl]methyl,
      —C₀₋₄alkyl-N(Rₐ)(R_{b}), for example —C₀alkyl-N(Rₐ)(R_{b}) or —C₁alkyl-N(Rₐ)(R_{b}),
      halo (e.g., Cl),
      —N(Rₑ)—C(O)—O—C₁₋₄alkyl (e.g., —N(H)—C(O)—O—C(H)(CH₃)CH₃),
      —C₁₋₆alkyl-OC₁₋₄alkyl (e.g., —CH₂CH₂CH₂CH₂—O—CH₃),
      —O-haloC₁₋₄alkyl (e.g., —OCH₂CF₃),
      —CH₂—O—C(O)—C₁₋₄alkyl (e.g., —CH₂—O—C(O)—CH₃),
      —C(O)O—C₁₋₄alkyl (e.g., —C(O)OCH₃); or
   (vi) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[1.10.2.25,8.016,24.018,23]heptacosa-5,7,13(25),14,16(24),17,22,26-octaene-19,21-dione;
   (vii) Rₐ is H and R_{b} is:
      C₁₋₄alkyl (e.g., methyl),
      C₁₋₄alkoxy-C₁₋₄alkyl (e.g., methoxyethyl),
   or Rₐ and R_{b} are independently:
      C₁₋₄alkyl (e.g., methyl),
      C₁₋₄alkoxy-C₁₋₄alkyl (e.g., methoxyethyl),
   (viii) R_{c} and R_{d} are independently H, C₁₋₄alkyl (e.g., methyl) or arylC₁₋₄alkyl (e.g., benzyl);
   (ix) R₃ and R₄ are independently H or C₁₋₄alkyl (e.g., methyl);
   (x) Rₑ is H or C₁₋₄alkyl,
P.6. the compound of formula P or any of P.1-P.5, wherein:
   Alk is C₂₋₃alkylene (e.g., ethylene, i.e., —CH₂CH₂—, or n-propylene, i.e. —CH₂CH₂CH₂—) optionally substituted with one or more C₁₋₄alkyl(e.g., methyl or ethyl) or arylC₁₋₄alkyl (e.g., benzyl); or
   Alk is C₂₋₃alkylene (e.g., ethylene, i.e., —CH₂CH₂—, or n-propylene, i.e., —CH₂CH₂CH₂—) optionally substituted with one hydroxy or C₁₋₄alkoxy (e.g., ethoxy or isopropyloxy) group; and
   X is a single bond, —S— or —O—;
   A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, t-butyl), and/or
      halo (e.g., Cl, F),
   R₁ is:
      C₁₋₆alkyl, e.g., C₁₋₄alkyl (for example, methyl, ethyl, n-propyl, isopropyl, isobutyl, 1-methylpropyl), or C₃₋₈cycloalkyl (e.g., cyclopentyl),
   R₂ is:
      H,
      C₁₋₆alkyl, e.g., C₁₋₄alkyl (for example, methyl, ethyl, n-propyl, isobutyl, n-hexyl),
      —C₀₋₄alkyl-C₃₋₈cycloalkyl (e.g., cyclopropyl),
   R₃ and itₜ are H;
P.7. the compound of formula P or any of P.1-P.6, wherein:
   Alk is C₂₋₃alkylene (e.g., ethylene, i.e., —CH₂CH₂—, or n-propylene, i.e. —CH₂CH₂CH₂—) optionally substituted with one or more C₁₋₄-alkyl (e.g., methyl or ethyl); or
   Alk is C₂₋₃alkylene (e.g., ethylene, i.e., —CH₂CH₂—, or n-propylene, i.e., —CH₂CH₂CH₂—) optionally substituted with one C₁₋₄alkoxy (e.g., ethoxy or isopropyloxy) group; and
   X is a single bond and A is aryl (e.g., phenyl), wherein the aryl group is optionally substituted with one or more
      C₁₋₄alkyl (e.g., methyl, t-butyl), and/or
      halo (e.g., Cl, F), or
   R₁ is C₁₋₆alkyl, e.g., C₁₋₄alkyl (for example, methyl, ethyl, n-propyl, isopropyl, isobutyl, 1-methylpropyl), or
      C₃₋₈cycloalkyl (e.g., cyclopentyl),
   R₂ is:
      H,
      C₁₋₆alkyl, e.g., C₁₋₄alkyl (for example, methyl, ethyl, n-propyl, isobutyl, n-hexyl),
      —C₀₋₄alkyl-C₃₋₈cycloalkyl (e.g., cyclopropyl),
   R₃ and R₄ are H;
P.8. the compound of Formula P or any of formulae P.1-P.7 wherein:
   Alk is n-propylene, i.e., —CH₂CH₂CH₂—;
   X is a single bond;
   A is phenyl optionally substituted with one or more C₁₋₄alkyl (e.g., methyl, t-butyl) or halo (e.g., Cl, F);
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl), or
      Cyclopentyl,
   R₂ is:
      H,
      C₁₋₆alkyl, e.g., C₁₋₄alkyl (for example, methyl, n-propyl, isobutyl, n-hexyl),
   R₃ and R₄ are H;
P.9. the compound of Formula P or any of formulae P.1-P.8 wherein:
   Alk is n-propylene;
   X is a single bond;
   A is phenyl;
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
   R₂ is:
      H,
      C₁₋₆alkyl, e.g., C₁₋₄alkyl (for example, methyl, n-propyl, isobutyl, n-hexyl),
   R₃ and R₄ are H;
P.10. the compound of Formula P or any of formulae P.1-P.9 wherein:
   Alk is n-propylene;
   X is a single bond;
   A is phenyl substituted with one or more C₁₋₄alkyl (e.g., methyl, t-butyl) or halo (e.g., Cl, F);
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
   R₂ is:
      H,
      C₁₋₆alkyl, e.g., C₁₋₄alkyl (for example, methyl, n-propyl, isobutyl, n-hexyl),
   R₃ and R₄ are H;
P.11. The compound according to any of the preceding formulae, wherein:
   Alk is C₂₋₃alkylene (e.g., ethylene, i.e., CH₂CH₂—, n-propylene, i.e., —CH₂CH₂CH₂—) optionally substituted with one or more C₁₋₄alkyl (e.g., methyl, ethyl or isobutyl); or
   Alk is C₂₋₃alkylene (e.g., ethylene, i.e., CH₂CH₂— or n-propylene, i.e., —CH₂CH₂CH₂—) optionally substituted with one C₁₋₄alkoxy (e.g., ethoxy or isopropyloxy) group;
   X is a single bond, —S— or —O—;
   A is aryl (e.g., phenyl), wherein the aryl group is optionally substituted with one or more
      C₁₋₄alkyl (e.g., methyl),
      halo (e.g., Cl, F),
   R₁ is:
      C₁₋₆alkyl, e.g., C₁₋₄alkyl (for example, methyl, ethyl, n-propyl, isopropyl, isobutyl, t-butyl, 1-methylpropyl), C₃₋₈cycloalkyl (e.g., cyclopentyl),
   R₂ is:
      H,
      C₁₋₆alkyl, e.g., C₁₋₄alkyl (for example, methyl, ethyl, n-propyl or isopropyl),
      —C₀₋₄alkyl-C₃₋₈cycloalkyl (e.g., cyclopropyl),
P.12. The compound according to any of the preceding formulae, wherein:
   Alk is C₃alkylene (e.g., n-propylene, i.e., —CH₂CH₂CH₂—) optionally substituted with one or more C₁₋₄alkyl (e.g., methyl or ethyl); or
   Alk is C₃alkylene (e.g., n-propylene, i.e., —CH₂CH₂CH₂—) optionally substituted with one C₁₋₄alkoxy (e.g., ethoxy or isopropyloxy) group;
   X is a single bond;
   A is aryl (e.g., phenyl), wherein the aryl group is optionally substituted with one or more
      C₁₋₄alkyl (e.g., methyl),
      halo (e.g., Cl, F),
   R₁ is:
      C₁₋₆alkyl, e.g., C₁₋₄alkyl (for example, methyl, ethyl, n-propyl, isopropyl or 1-methylpropyl),
      C₃₋₈cycloalkyl (e.g., cyclopentyl),
   R₂ is:
      H,
      C₁₋₆alkyl, e.g., C₁₋₄alkyl (for example, methyl, ethyl, n-propyl or isopropyl),
      —C₀₋₄alkyl-C₃₋₈cycloalkyl (e.g., cyclopropyl),
P.13. The compound according to Formula P or any of the preceding formulae, wherein the substituents are as described in any one of formulae 1.1-1.107;
P.14. The compound according to any of the preceding formulae, wherein the compound is selected from those set forth in formula 1.27 and:

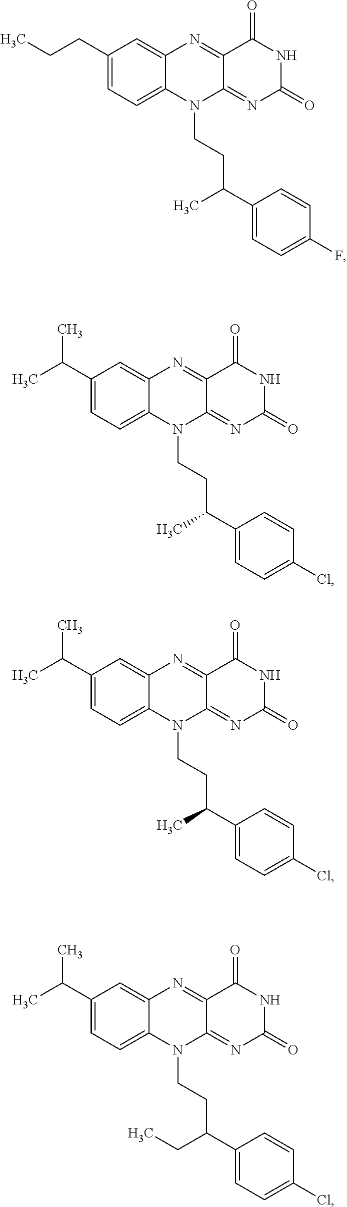 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1]

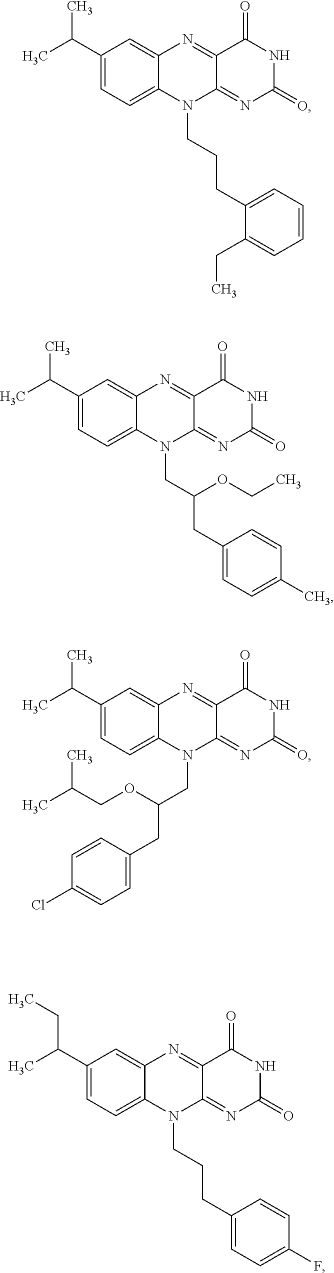 [SMILES: CCc1ccccc1CCCn1c2nc(=O)[nH]c(=O)c-2nc2cc(C(C)C)ccc21 ; CCOC(Cc1ccc(C)cc1)Cn1c2nc(=O)[nH]c(=O)c-2nc2cc(C(C)C)ccc21 ; CC(C)COC(Cc1ccc(Cl)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1]

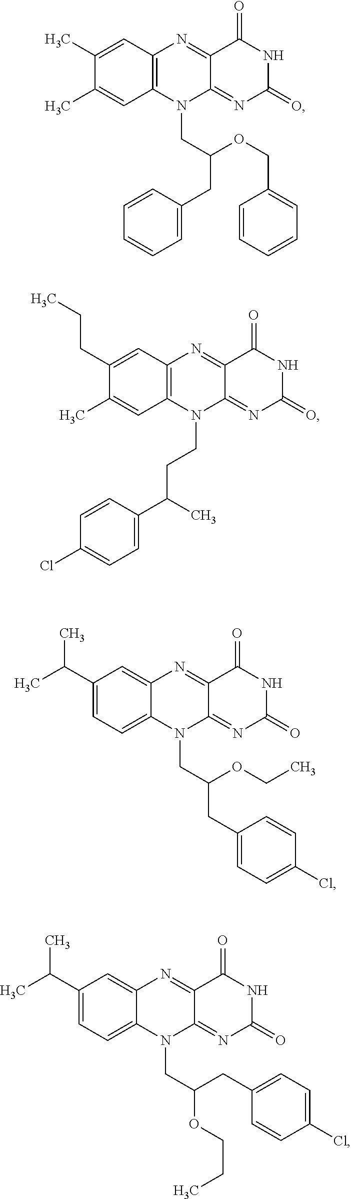 [SMILES: Cc1cc2nc3c(=O)[nH]c(=O)nc-3n(CC(Cc3ccccc3)OCc3ccccc3)c2cc1C ; CCCc1cc2nc3c(=O)[nH]c(=O)nc-3n(CCC(C)c3ccc(Cl)cc3)c2cc1C ; CCOC(Cc1ccc(Cl)cc1)Cn1c2nc(=O)[nH]c(=O)c-2nc2cc(C(C)C)ccc21 ; CCCOC(Cc1ccc(Cl)cc1)Cn1c2nc(=O)[nH]c(=O)c-2nc2cc(C(C)C)ccc21]

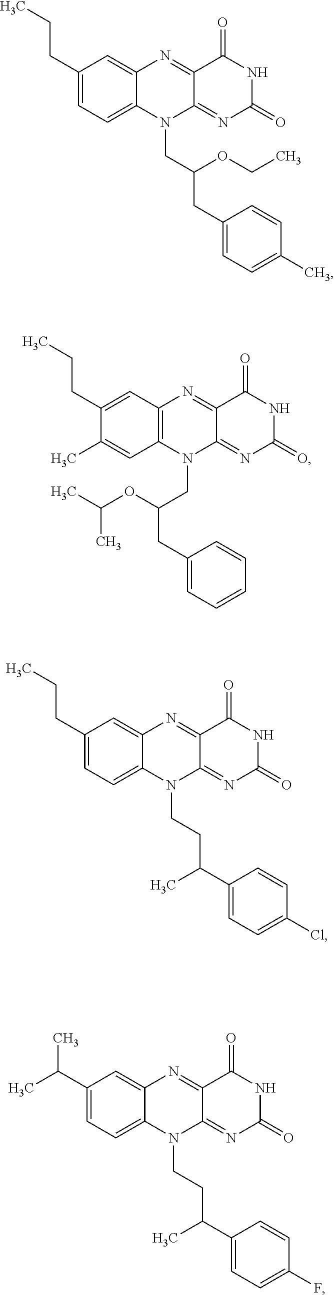 [SMILES: CCCc1ccc2c(c1)nc1c(=O)[nH]c(=O)nc-1n2CC(Cc1ccc(C)cc1)OCC ; CCCc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCC(C)c1ccc(Cl)cc1 ; CC(C)c1ccc2c(c1)nc1c(=O)[nH]c(=O)nc-1n2CCC(C)c1ccc(F)cc1]

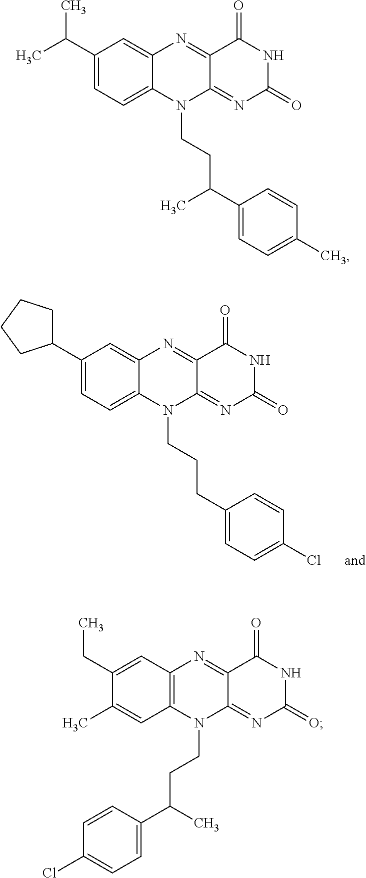 [SMILES: Cc1ccc(C(C)CCn2c3nc(=O)[nH]c(=O)c-3nc3cc(C(C)C)ccc32)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C]

P.15. The compound according to any of the preceding formulae wherein the compound is selected from formula 1.28 and:

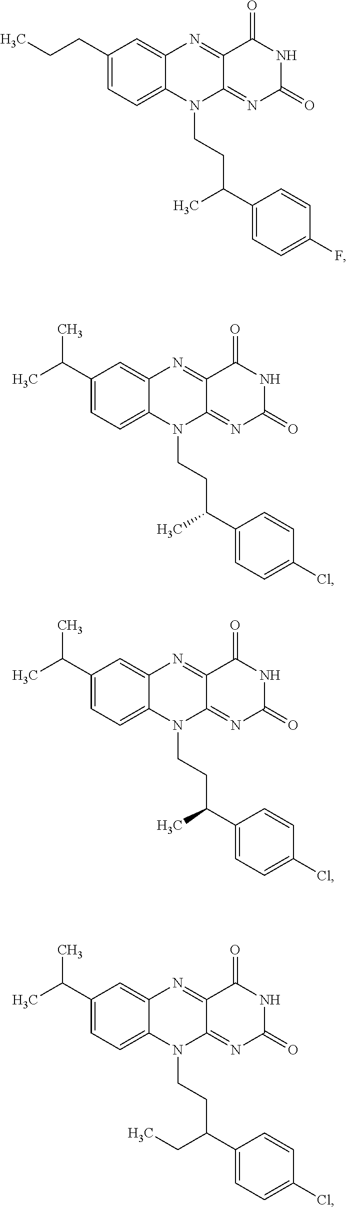 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1]

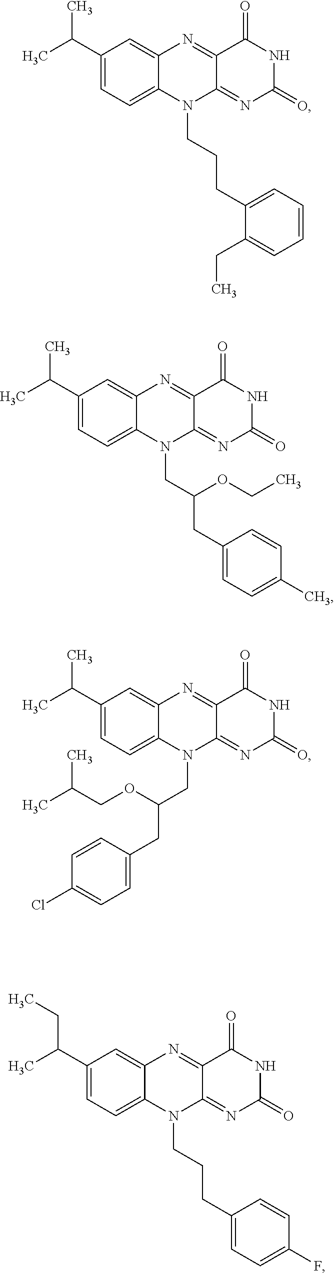 [SMILES: CCc1ccccc1CCCn1c2nc(=O)[nH]c(=O)c-2nc2cc(C(C)C)ccc21 ; CCOC(Cc1ccc(C)cc1)Cn1c2nc(=O)[nH]c(=O)c-2nc2cc(C(C)C)ccc21 ; CC(C)COC(Cc1ccc(Cl)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1]

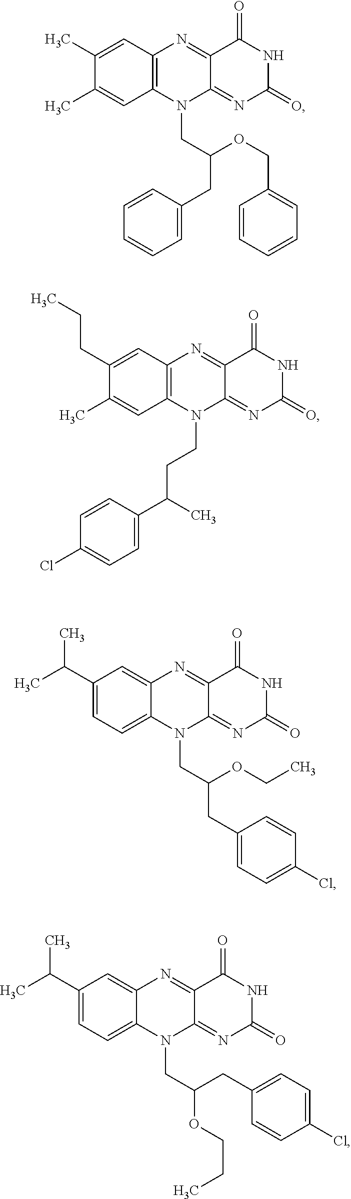 [SMILES: Cc1cc2nc3c(=O)[nH]c(=O)nc-3n(CC(Cc3ccccc3)OCc3ccccc3)c2cc1C ; CCCc1cc2nc3c(=O)[nH]c(=O)nc-3n(CCC(C)c3ccc(Cl)cc3)c2cc1C ; CCOC(Cc1ccc(Cl)cc1)Cn1c2nc(=O)[nH]c(=O)c-2nc2cc(C(C)C)ccc21 ; CCCOC(Cc1ccc(Cl)cc1)Cn1c2nc(=O)[nH]c(=O)c-2nc2cc(C(C)C)ccc21]

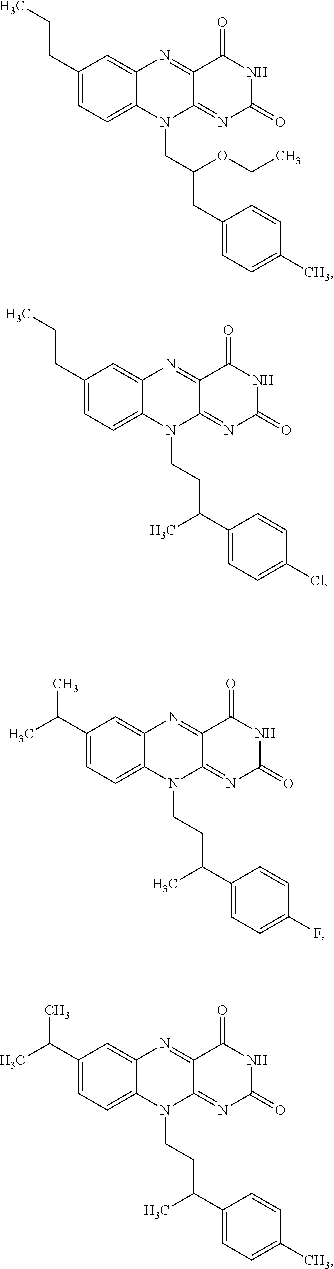 [SMILES: CCCc1ccc2c(c1)nc1c(=O)[nH]c(=O)nc-1n2CC(Cc1ccc(C)cc1)OCC ; CCCc1ccc2c(c1)nc1c(=O)[nH]c(=O)nc-1n2CCC(C)c1ccc(Cl)cc1 ; CC(C)c1ccc2c(c1)nc1c(=O)[nH]c(=O)nc-1n2CCC(C)c1ccc(F)cc1 ; Cc1ccc(C(C)CCn2c3nc(=O)[nH]c(=O)c-3nc3cc(C(C)C)ccc32)cc1]

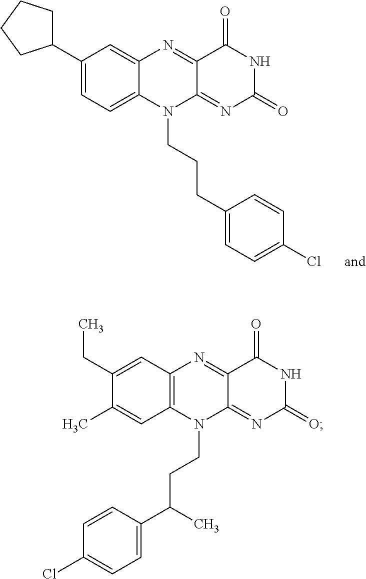 [SMILES: O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C]

P.16. The compound according to any of the preceding formulae wherein the compound is selected from:

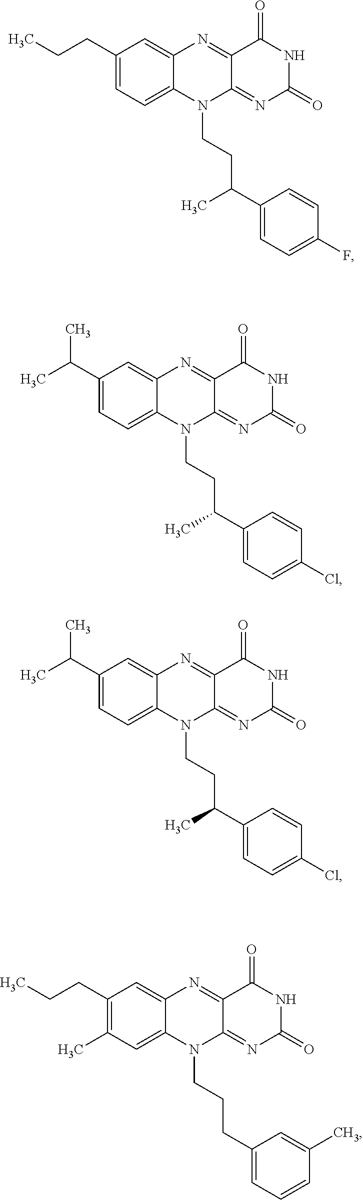 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCCc1cc2nc3c(=O)[nH]c(=O)nc-3n(CCCc3cccc(C)c3)c2cc1C]

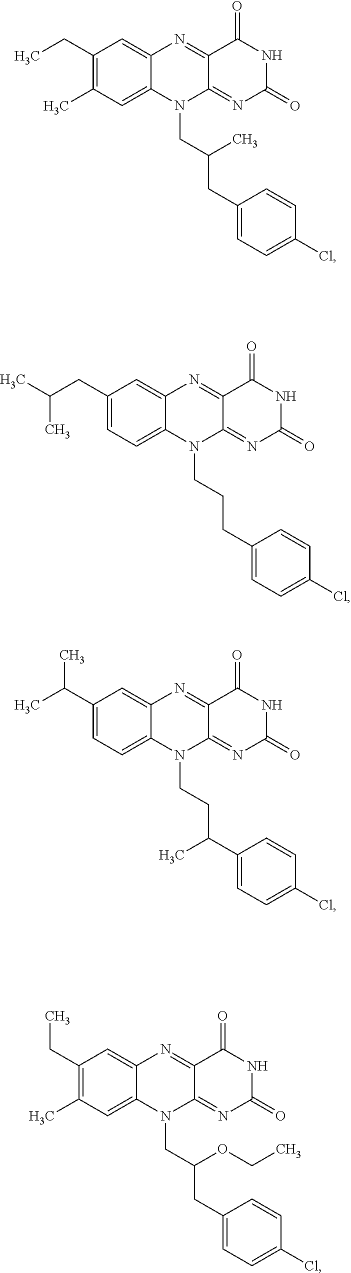 [SMILES: CCc1cc2nc3c(=O)[nH]c(=O)nc-3n(CC(C)Cc3ccc(Cl)cc3)c2cc1C ; CC(C)Cc1ccc2c(c1)nc1c(=O)[nH]c(=O)nc-1n2CCCc1ccc(Cl)cc1 ; CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21]

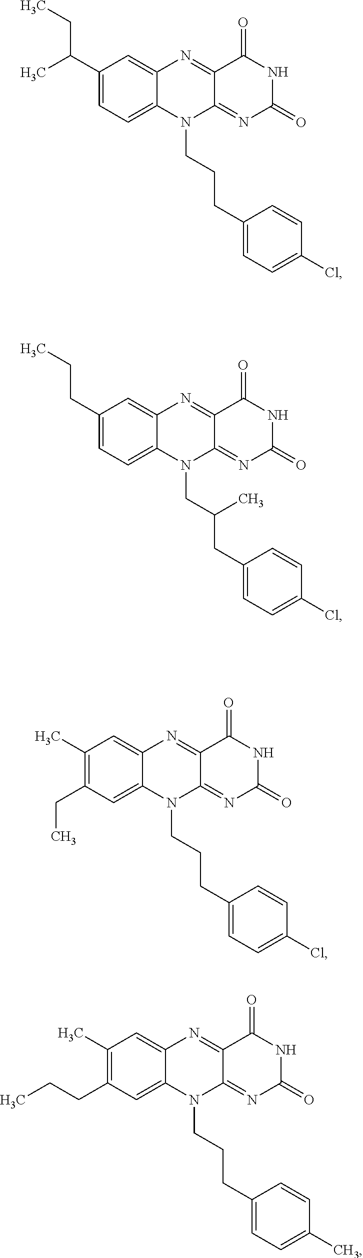 [SMILES: CCC(C)c1ccc2c(c1)nc1c(=O)[nH]c(=O)nc-1n2CCCc1ccc(Cl)cc1 ; CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1 ; CCCc1cc2c(cc1C)nc1c(=O)[nH]c(=O)nc-1n2CCCc1ccc(C)cc1]

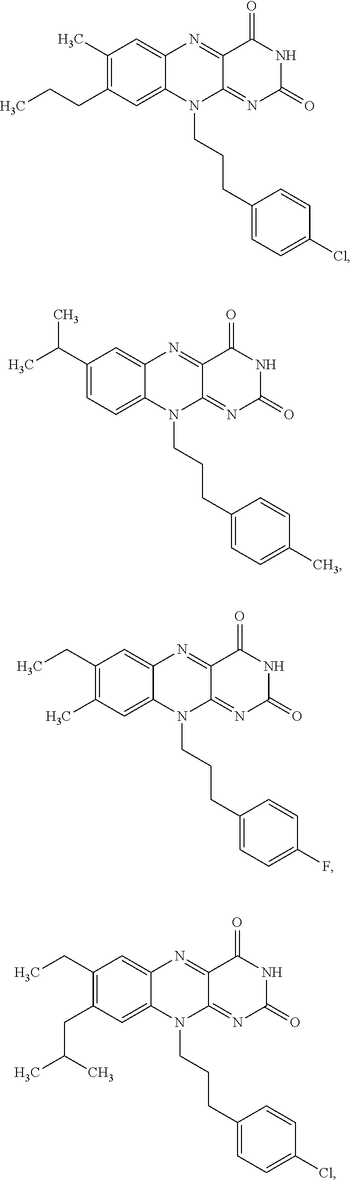 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; CCc1cc2nc3c(=O)[nH]c(=O)nc-3n(CCCc3ccc(Cl)cc3)c2cc1CC(C)C]

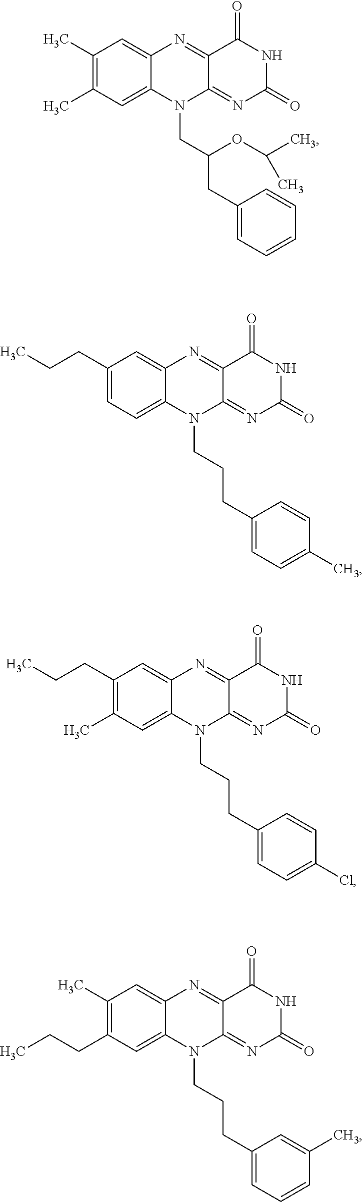 [SMILES: Cc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCCc1ccc(C)cc1 ; CCCc1cc2nc3c(=O)[nH]c(=O)nc-3n(CCCc3ccc(Cl)cc3)c2cc1C ; CCCc1cc2c(cc1C)nc1c(=O)[nH]c(=O)nc-1n2CCCc1cccc(C)c1]

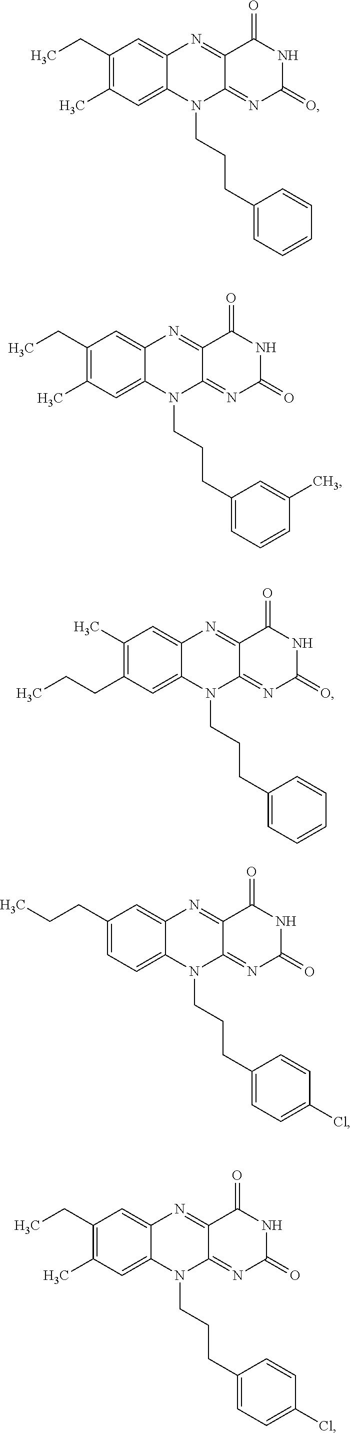

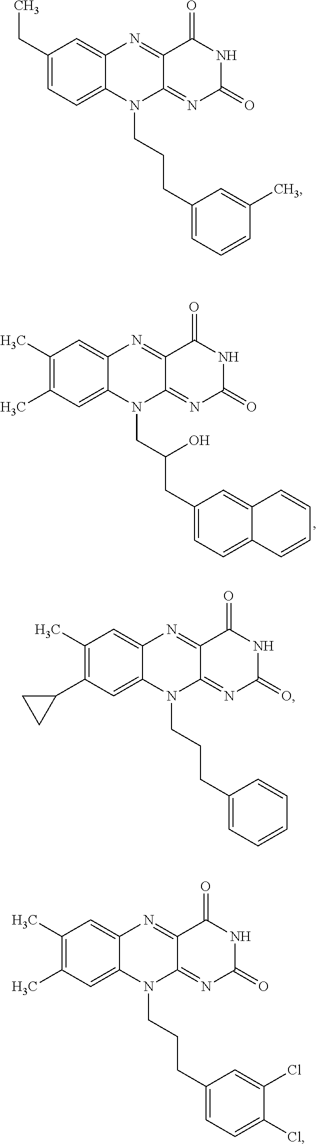

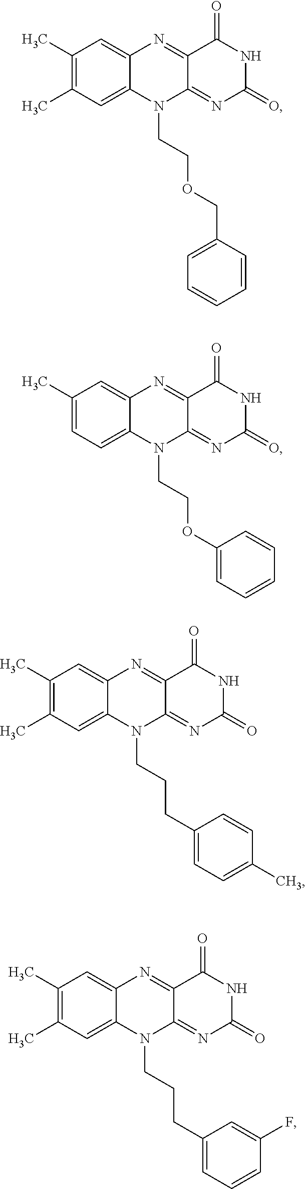

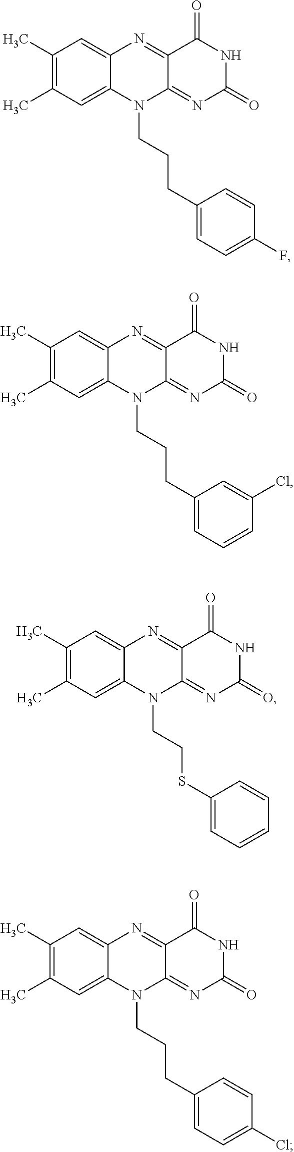 [SMILES: Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3ccc(Cl)cc3)c2cc1C]

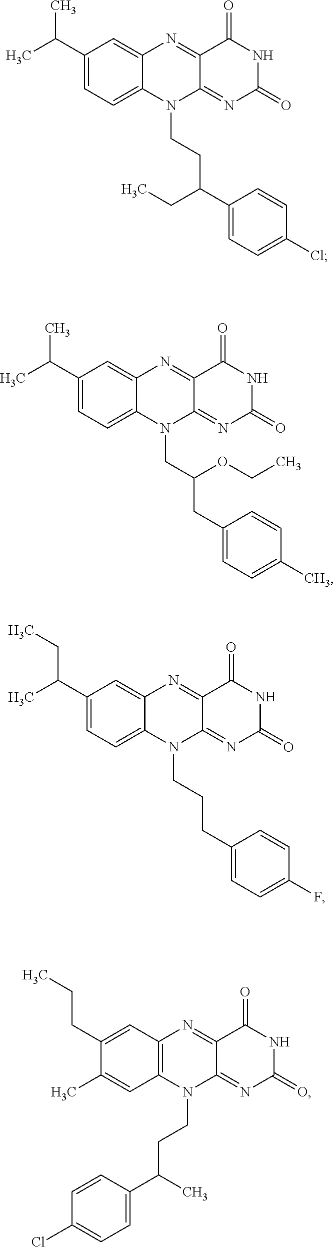 [SMILES: CCC(CCn1c2nc(=O)[nH]c(=O)c-2nc2cc(C(C)C)ccc21)c1ccc(Cl)cc1 ; CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C]

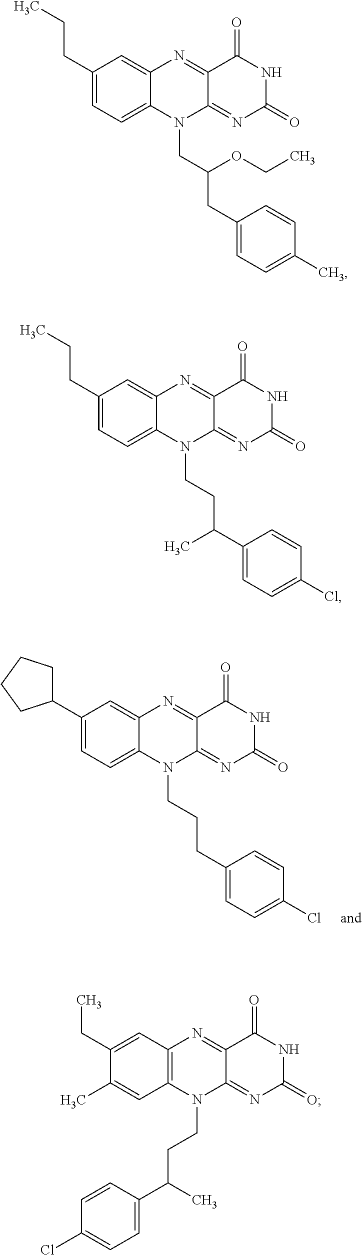 [SMILES: CCCc1ccc2c(c1)nc1c(=O)[nH]c(=O)nc-1n2CC(Cc1ccc(C)cc1)OCC ; CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C]

P.17. The compound according to any of the preceding formulae wherein the compound is selected from formula 1.31 and:

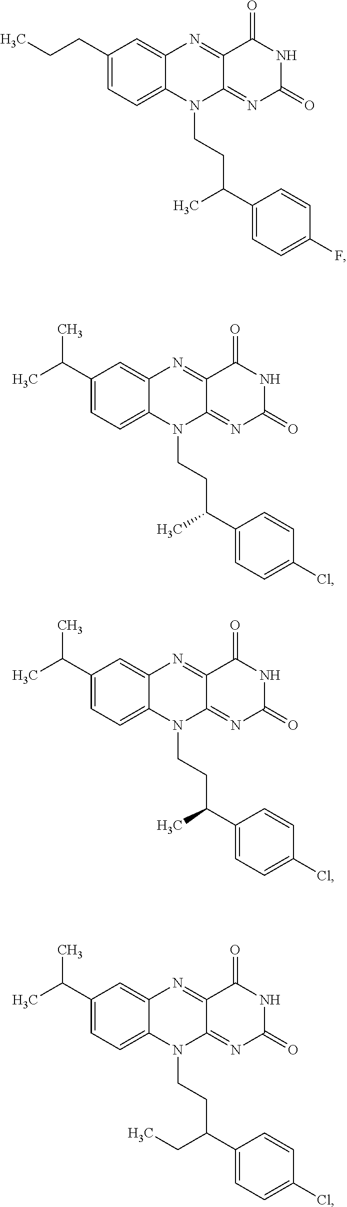 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1]

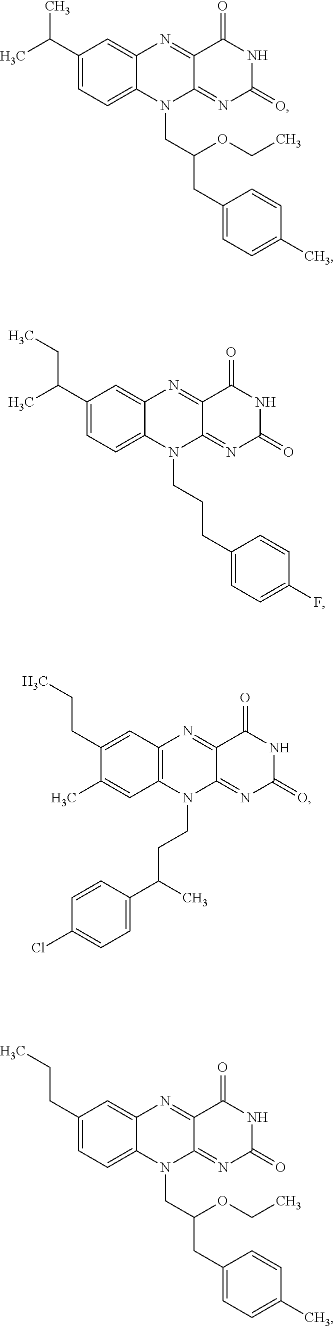 [SMILES: CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C ; CCCc1ccc2c(c1)nc1c(=O)[nH]c(=O)nc-1n2CC(Cc1ccc(C)cc1)OCC]

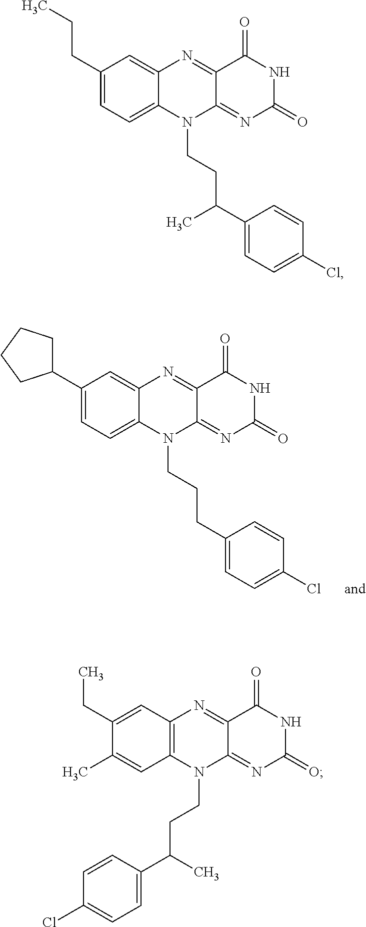 [SMILES: CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C]

in free or salt form.

The invention further relates to a compound of Formula Q as described in the following formulae:
1.1. the compound of formula Q, wherein
   (i) Alk is C₁₋₆alkylene (e.g., C₂₋₅alkylene, for example ethylene, i.e., CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl or isobutyl) and/or —N(R_{c})(R_{d}); or
      Alk is C₁₋₆alkylene (e.g., C₂₋₅alkylene, for example n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one hydroxy or C₁₋₄alkoxy (e.g., methoxy, ethoxy or isopropyloxy) group;
   (ii) X is a single bond, —S—, —S(O)₂—, —S(O)— or —O—;
   (iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, t-butyl or n-prop-2-en-1-yl),
      C₁₋₄alkoxy (e.g., methoxy),
      hydroxy,
      —O—C₁₋₄alkyl-N(R_{c})(R_{d}), for example —OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      —O—(CH₂CH₂O)₁₋₃—C₁₋₄alkyl (e.g., —OCH₂CH₂OCH₃ or —O(CH₂CH₂O)₃CH₃), and/or
      —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl);
   (iv) R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
      C₃₋₈cycloalkyl (e.g., cyclopropyl),
      aryl (e.g., phenyl), or
      C₁₋₄alkoxy (e.g., methoxy);
   (v) R₂ is:
      H,
      C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
      —C₀₋₄alkyl-C₃₋₈cycloalkyl (e.g., cyclopropyl),
      —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
      —C₀₋₄alkyl-N(Rₐ)(R_{b}), for example —C₀alkyl-N(Rₐ)(R_{b}) or
      —C₁alkyl-N(Rₐ)(R_{b}),
      C₁₋₄alkoxy (e.g., methoxy),
      halo (e.g., Cl),
      —O—(CH₂CH₂O)₁₋₃—C₁₋₄alkyl (e.g., —OCH₂CH₂OCH₃ or —O(CH₂CH₂O)₃CH₃),
      —N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
      —N(Rₑ)—C(O)—O—C₄alkyl (e.g., —N(H)—C(O)—O—C(H)(CH₃)CH₃),
      —N(Rₑ)—C(O)-aryl wherein said aryl is optionally substituted with one or more halo (e.g., F), for example —N(H)—C(O)-(4-fluorophenyl),
      —C₁₋₆alkyl-OC₁₋₄alkyl (e.g., —CH₂CH₂CH₂CH₂—O—CH₃),
      —O—CH₂CH₂—O—CH₂-phenyl,
      —O-haloC₁₋₄alkyl (e.g., —OCH₂CF₃),
      —CH₂—O—C(O)—C₁₋₄alkyl (e.g., —CH₂—O—C(O)—CH₃),
      —C(O)O—C₁₋₄alkyl (e.g., —C(O)OCH₃), or
      C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
   (vi) Optionally, R₁ and R₂ are linked together so that together with the carbon atoms to which they are attached they form a cyclic structure (e.g., R₁ and R₂ are linked together to form ethylenedioxy);
   (vii) Rₐ and R_{b} are independently:
      H,
      C₁₋₄alkyl (e.g., methyl) optionally substituted with one or more hydroxy groups for example, 2,3-dihydroxyprop-1-yl,
      C₃₋₈cycloalkyl (e.g., cyclopropyl or cyclopentyl),
      C₁₋₄alkoxy-C₁₋₄alkyl (e.g., methoxyethyl),
      hydroxy-C₁₋₄alkyl (e.g., hydroxyethyl),
      N(R_{c})(R_{d})—C₁₋₄alkyl (e.g., dimethylaminoethyl);
   (viii) R_{c} and R_{d} are independently H, C₁₋₄alkyl (e.g., methyl) or arylC₁₋₄alkyl (e.g., benzyl);
   (ix) R₃ and R₄ are independently H or C₁₋₄alkyl (e.g., methyl);
   (x) Rₑ is H or C₁₋₄alkyl;
1.2. the compound of formula Q or 1.1, wherein R₂ is:
   H,
   C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
   —C₀₋₄alkyl-C₃₋₈cycloalkyl (e.g., cyclopropyl),
   —C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
   —C₀₋₁alkyl-N(Rₐ)(R_{b}), wherein Rₐ is H and R_{b} is C₁₋₄alkoxy-C₁₋₄alkyl (e.g., methoxyethyl) or both Rₐ and R_{b} are methyl,
   C₁₋₄alkoxy (e.g., methoxy),
   halo (e.g., Cl),
   —C₁₋₆alkyl-OC₁₋₄alkyl (e.g., —CH₂CH₂CH₂CH₂—O—CH₃),
   —O-haloC₁₋₄alkyl (e.g., —OCH₂CF₃),
   —CH₂—O—C(O)—C₁₋₄alkyl (e.g., —CH₂—O—C(O)—CH₃),
   —C(O)O—C₁₋₄ alkyl (e.g., —C(O)OCH₃), or
   C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
1.3. the compound of formula Q, 1.1 or 1.2, wherein:
   (ii) Alk is C₁₋₄alkylene (e.g., C₂₋₅alkylene, for example ethylene, i.e., —CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl or isobutyl); or
      Alk is C₁₋₄alkylene (e.g., C₂₋₅alkylene, for example n-propylene, i.e., —CH₂CH₂CH₂—, n-butylene, i.e., —CH₂CH₂CH₂CH₂—, n-pentylene, i.e., —CH₂CH₂CH₂CH₂CH₂—) optionally substituted with one hydroxy or C₁₋₄alkoxy (e.g., methoxy, ethoxy or isopropyloxy) group;
1.4. the compound of formula Q, 1.1, 1.2 or 1.3, wherein:
   A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, t-butyl or n-prop-2-en-1-yl),
      C₁₋₄alkoxy (e.g., methoxy),
      hydroxy,
      —O—C₁₋₄alkyl-N(R_{c})(R_{d}), for example —OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      —OCH₂CH₂OCH₃, and/or
      —CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl);
1.5. the compound of formula Q or any of 1.1-1.3, wherein R₃ and R₄ are H,
1.6. the compound of formula Q or any of 1.1-1.3, wherein R₃ or R₄ is C₁₋₄alkyl (e.g., methyl);
1.7. the compound of formula Q wherein:
   (i) Alk is C₁₋₆alkylene (e.g., C₂₋₅alkylene, for example ethylene, i.e., —CH₂CH₂—, n-propylene, i.e., —CH₂CH₂CH₂—, n-butylene, i.e., —CH₂CH₂CH₂CH₂—, or n-pentylene, i.e., —CH₂CH₂CH₂CH₂CH₂—) optionally substituted with one or more C₁₋₄alkyl (e.g., methyl or isobutyl) and/or —N(R_{c})(R_{d}); or
      Alk is C₁₋₆alkylene (e.g., C₂₋₅alkylene, for example n-propylene, i.e., —CH₂CH₂CH₂—, n-butylene, i.e., —CH₂CH₂CH₂CH₂—, n-pentylene, i.e., —CH₂CH₂CH₂CH₂CH₂—) optionally substituted with one hydroxy or C₁₋₄alkoxy (e.g., methoxy, ethoxy or isopropyloxy) group; and
   (ii) X is a single bond, —S— or —O—;
   (iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, t-butyl),
      —O—C₁₋₄alkyl-N(R_{c})(R_{d}), for example —OCH₂CH₂N(CH₃)₂,
      halo (e.g., Cl, F),
      haloC₁₋₄alkyl (e.g., CF₃),
      —O-haloC₁₋₄alkyl (e.g., —OCF₃),
      cyano,
      [2,6-dimethylmorpholin-4-yl]methyl, e.g. [(2R,6S)-2,6-dimethylmorpholin-4-yl]methyl);
   (iv) R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
      C₃₋₈cycloalkyl (e.g., cyclopropyl),
      aryl (e.g., phenyl), or
      C₁₋₄alkoxy (e.g., methoxy);
   (v) R₂ is:
      H,
      C₁₋₆alkyl, e.g., C₁₋₄alkyl (for example, methyl, ethyl, n-propyl, isopropyl, n-butyl, isobutyl, n-but-2-en-1-yl, n-hexyl),
      —C₀₋₄alkyl-C₃₋₈cycloalkyl (e.g., cyclopropyl),
      —C₁₋₄alkyl-heteroC₃₋₈cycloalkyl, wherein said heterocycloalkyl is optionally substituted with one or more C₁₋₄alkyl (e.g., methyl) groups, for example, [2,6-dimethylmorpholin-4-yl]methyl,
      —C₀₋₄alkyl-N(Rₐ)(R_{b}), for example —C₀alkyl-N(Rₐ)(R_{b}) or —C₁alkyl-N(Rₐ)(R_{b}),
      halo (e.g., Cl),
      —N(Rₑ)—C(O)—O—C₁₋₄alkyl (e.g., —N(H)—C(O)—O—C(H)(CH₃)CH₃),
      —C₁₋₆alkyl-OC₁₋₄alkyl (e.g., —CH₂CH₂CH₂CH₂—O—CH₃),
      —O-haloC₁₋₄alkyl (e.g., —OCH₂CF₃),
      —CH₂—O—C(O)—C₁₋₄alkyl (e.g., —CH₂—O—C(O)—CH₃),
      —C(O)O—C₁₋₄alkyl (e.g., —C(O)OCH₃); or
   (vi) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016, 24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
   (vii) Rₐ is H and R_{b} is:
      C₁₋₄alkyl (e.g., methyl),
      C₁₋₄alkoxy-C₁₋₄alkyl (e.g., methoxyethyl),
   or Rₐ and R_{b} are independently:
      C₁₋₄alkyl (e.g., methyl),
      C₁₋₄alkoxy-C₁₋₄alkyl (e.g., methoxyethyl),
   (viii) R_{c} and R_{d} are independently H, C₁₋₄alkyl (e.g., methyl) or arylC₁₋₄alkyl (e.g., benzyl);
   (ix) R₃ and R₄ are independently H or C₁₋₄alkyl (e.g., methyl);
   (x) Rₑ is H or C₁₋₄alkyl,
1.8. the compound of formula Q or any of 1.1-1.7, wherein:
   Alk is C₂₋₃alkylene (e.g., ethylene, i.e., —CH₂CH₂—, or n-propylene, i.e. —CH₂CH₂CH₂—) optionally substituted with one or more C₁₋₄alkyl (e.g., methyl); or
   Alk is C₂₋₃alkylene (e.g., ethylene, i.e., —CH₂CH₂—, or n-propylene, i.e., —CH₂CH₂CH₂—) optionally substituted with one hydroxy or C₁₋₄alkoxy (e.g., ethoxy or isopropyloxy) group; and
   X is a single bond, —S— or —O—;
   A is aryl (e.g., phenyl or naphthyl) or aryl-C₁₋₄alkyl (e.g., benzyl or naphthylmethyl), wherein the aryl group of said aryl or arylalkyl is optionally substituted with one or more
      C₁₋₄alkyl (e.g., methyl, t-butyl), and/or
      halo (e.g., Cl, F),
   R₁ is:
      C₁₋₆alkyl, e.g., C₁₋₄alkyl (for example, methyl, ethyl, n-propyl, isopropyl, isobutyl, 1-methylpropyl),
   R₂ is:
      H,
      C₁₋₆alkyl, e.g., C₁₋₄alkyl (for example, methyl, ethyl, n-propyl, isobutyl, n-hexyl),
      —C₀₋₄alkyl-C₃₋₈cycloalkyl (e.g., cyclopropyl),
   R₃ and R₄ are H;
1.9. the compound of formula Q or any of 1.1-1.8, wherein:
   Alk is C₂₋₃alkylene (e.g., ethylene, i.e., —CH₂CH₂—, or n-propylene, i.e. —CH₂CH₂CH₂—) optionally substituted with one or more C₁₋₄-alkyl (e.g., methyl); or
   Alk is C₂₋₃alkylene (e.g., ethylene, i.e., —CH₂CH₂—, or n-propylene, i.e., —CH₂CH₂CH₂—) optionally substituted with one C₁₋₄alkoxy (e.g., ethoxy or isopropyloxy) group; and
   X is a single bond and A is aryl (e.g., phenyl), wherein the aryl group is optionally substituted with one or more
      C₁₋₄alkyl (e.g., methyl, t-butyl), and/or
      halo (e.g., Cl, F), or
   R₁ is C₁₋₆alkyl, e.g., C₁₋₄alkyl (for example, methyl, ethyl, n-propyl, isopropyl, isobutyl, 1-methylpropyl),
   R₂ is:
      H,
      C₁₋₆alkyl, e.g., C₁₋₄alkyl (for example, methyl, ethyl, n-propyl, isobutyl, n-hexyl),
      —C₀₋₄alkyl-C₃₋₈cycloalkyl (e.g., cyclopropyl),
   R₃ and R₄ are H,
1.10. the compound of any one of formulae 1.1-1.9, wherein X is a single bond;
1.11. the compound of any one of formulae 1.1-1.8, wherein X is —S—;
1.12. the compound of any one of formulae 1.1-1.9, wherein X is —O—;
1.13. the compound of Formula Q or any of formulae 1.1-1.12, wherein Alk is n-propylene, i.e., —CH₂CH₂CH₂—;
1.14. the compound of Formula Q or any of formulae 1.1-1.13 wherein:
   Alk is n-propylene, i.e., —CH₂CH₂CH₂—;
   X is a single bond;
   A is phenyl optionally substituted with one or more C₁₋₄alkyl (e.g., methyl, t-butyl) or halo (e.g., Cl, F);
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
   R₂ is:
      H,
      C₁₋₆alkyl, e.g., C₁₋₄alkyl (for example, methyl, n-propyl, isobutyl, n-hexyl),
   R₃ and R₄ are H,
1.15. the compound of Formula Q or any of formulae 1.1-1.10 or 1.13-1.14 wherein:
   Alk is n-propylene;
   X is a single bond;
   A is phenyl;
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
   R₂ is:
      H,
      C₁₋₆alkyl, e.g., C₁₋₄alkyl (for example, methyl, n-propyl, isobutyl, n-hexyl),
   R₃ and R₄ are H,
1.16. the compound of Formula Q or any of formulae 1.1-1.10 or 1.13-1.15 wherein:
   Alk is n-propylene;
   X is a single bond;
   A is phenyl substituted with one or more C₁₋₄alkyl (e.g., methyl, t-butyl) or halo (e.g., Cl, F);
   R₁ is:
      H,
      C₁₋₆alkyl, e.g., C₁₋₄alkyl (for example, methyl, ethyl, n-propyl, isopropyl, isobutyl, t-butyl, 1-methylpropyl or n-hexyl),
   R₂ is:
      H,
      C₁₋₆alkyl, e.g., C₁₋₄alkyl (for example, methyl, n-propyl, isobutyl, n-hexyl),
   R₃ and R₄ are H;
1.17. the compound of Formula Q or any of formulae 1.1-1.16 wherein R₁ is C₁₋₆alkyl (e.g., methyl);
1.18. the compound of Formula Q or any of formulae 1.1-1.17 wherein R₂ is C₁₋₆alkyl (e.g., methyl);
1.19. the compound of Formula Q or any of formulae 1.1-1.18 wherein R₃ is H,
1.20. the compound of Formula Q or any of formulae 1.1-1.19 wherein R₄ is H,
1.21. the compound of Formula Q or any of formulae 1.1-1.20 wherein Rₑ and R_{d} are independently H or C₁₋₄alkyl (e.g., methyl);
1.22. the compound of Formula Q or any of formulae 1.1-1.21 wherein R_{c} and R_{d} are both H,
1.23. the compound of Formula Q or any of formulae 1.1-1.21 wherein R_{c} and R_{d} are both methyl;
1.24. the compound of Formula Q or any of formulae 1.1-1.23 wherein Rₑ is H or C₁₋₄alkyl;
1.25. the compound of Formula Q or any of formulae 1.1-1.24 wherein Rₑ is H,
1.26. the compound of Formula Q or any of formulae 1.1-1.24 wherein:
   Alk is C₁₋₆alkylene, e.g., C₂₋₃alkylene, preferably C₃alkylene (e.g., n-propylene, i.e., —CH₂CH₂CH₂—);
   X is a single bond;
   A is aryl (e.g., phenyl);
   R₁ is C₁₋₆alkyl, e.g., C₁₋₄alkyl (for example, methyl),
   R₂ is C₁₋₆alkyl, e.g., C₁₋₄alkyl (for example, methyl),
   R₃ and R₄ are H,
1.27. The compound of Formula Q according to any of the preceding formulae wherein said compound is selected from:

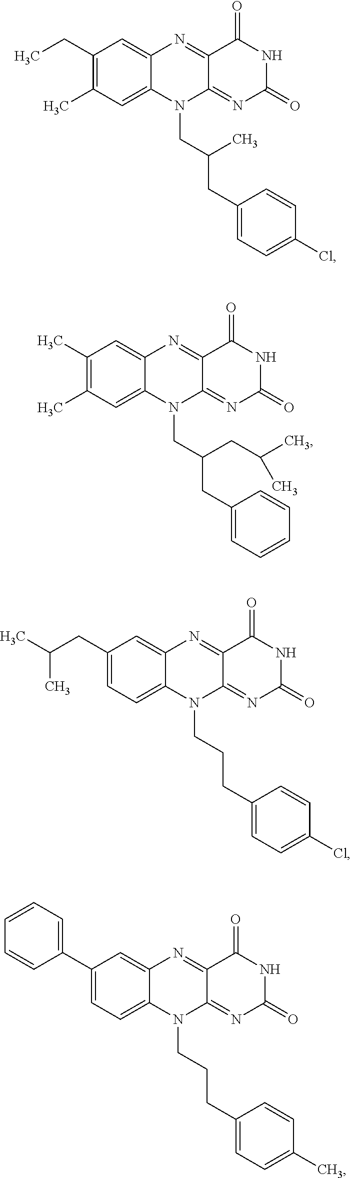

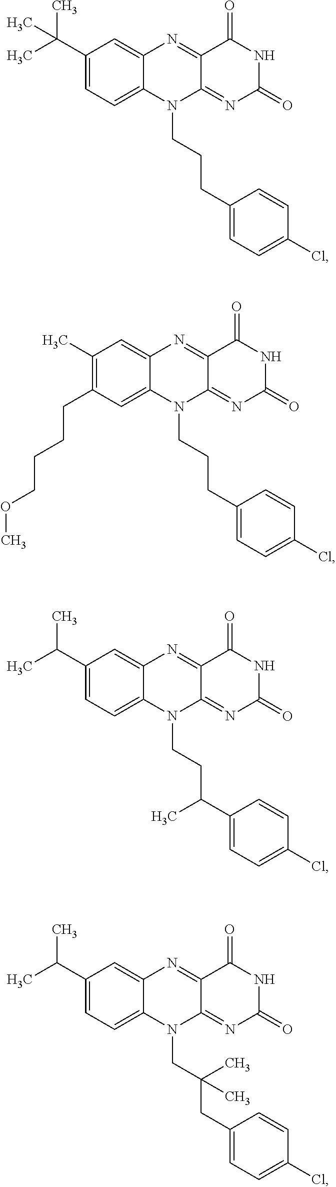 [SMILES: CC(C)(C)c1ccc2c(c1)nc1c(=O)[nH]c(=O)nc-1n2CCCc1ccc(Cl)cc1 ; COCCCCc1cc2c(cc1C)nc1c(=O)[nH]c(=O)nc-1n2CCCc1ccc(Cl)cc1 ; CC(C)c1ccc2c(c1)nc1c(=O)[nH]c(=O)nc-1n2CCC(C)c1ccc(Cl)cc1 ; CC(C)c1ccc2c(c1)nc1c(=O)[nH]c(=O)nc-1n2CC(C)(C)Cc1ccc(Cl)cc1]

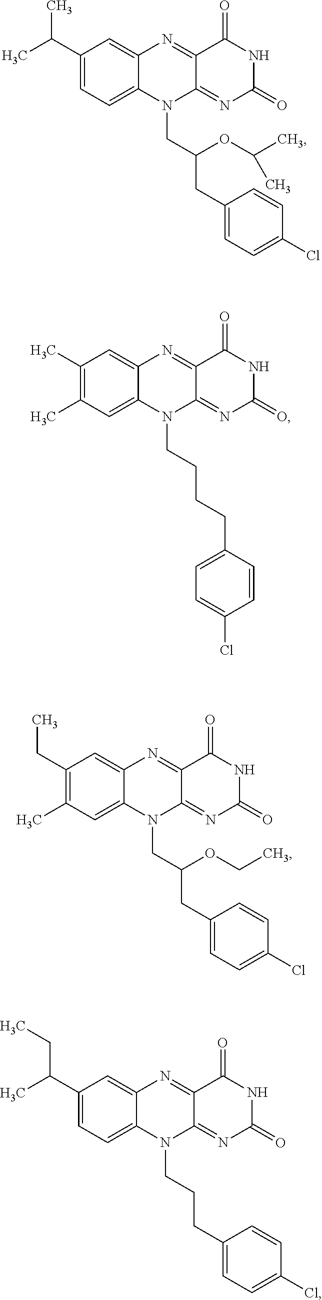 [SMILES: CC(C)OC(Cc1ccc(Cl)cc1)Cn1c2nc(=O)[nH]c(=O)c-2nc2cc(C(C)C)ccc21 ; Cc1cc2nc3c(=O)[nH]c(=O)nc-3n(CCCCc3ccc(Cl)cc3)c2cc1C ; CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1]

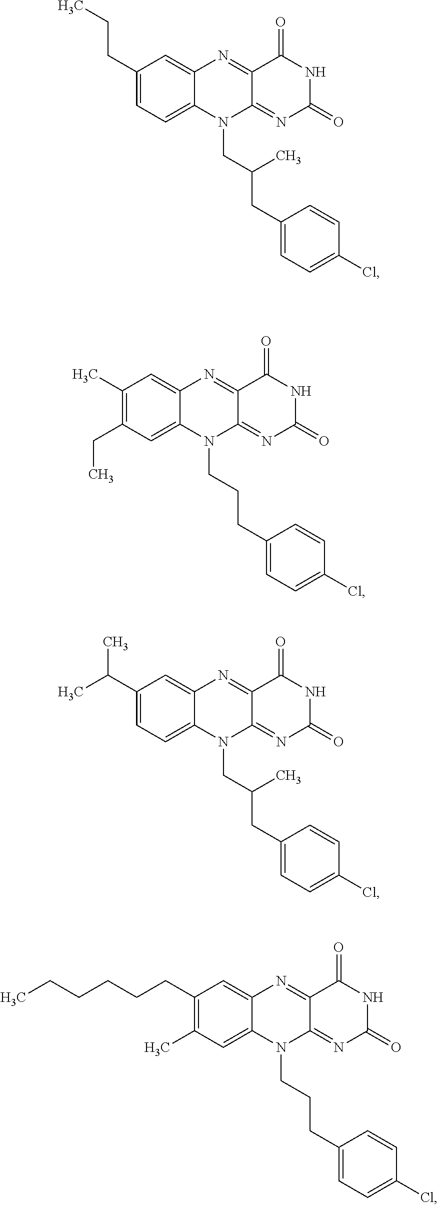 [SMILES: CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1 ; CC(Cc1ccc(Cl)cc1)Cn1c2nc(=O)[nH]c(=O)c-2nc2cc(C(C)C)ccc21 ; CCCCCCc1cc2nc3c(=O)[nH]c(=O)nc-3n(CCCc3ccc(Cl)cc3)c2cc1C]

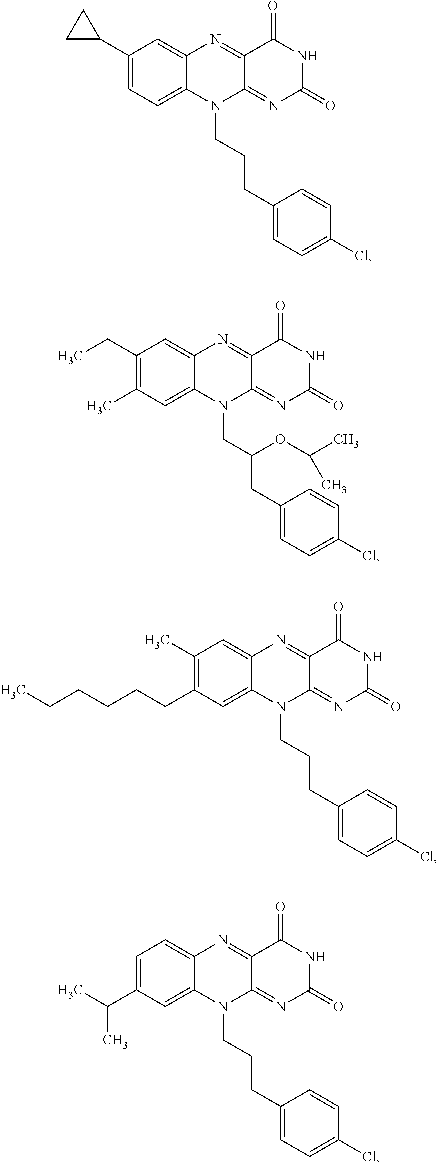 [SMILES: O=c1nc2n(CCCc3ccc(Cl)cc3)c3ccc(C4CC4)cc3nc-2c(=O)[nH]1 ; CCc1cc2nc3c(=O)[nH]c(=O)nc-3n(CC(Cc3ccc(Cl)cc3)OC(C)C)c2cc1C ; CCCCCCc1cc2c(cc1C)nc1c(=O)[nH]c(=O)nc-1n2CCCc1ccc(Cl)cc1 ; CC(C)c1ccc2nc3c(=O)[nH]c(=O)nc-3n(CCCc3ccc(Cl)cc3)c2c1]

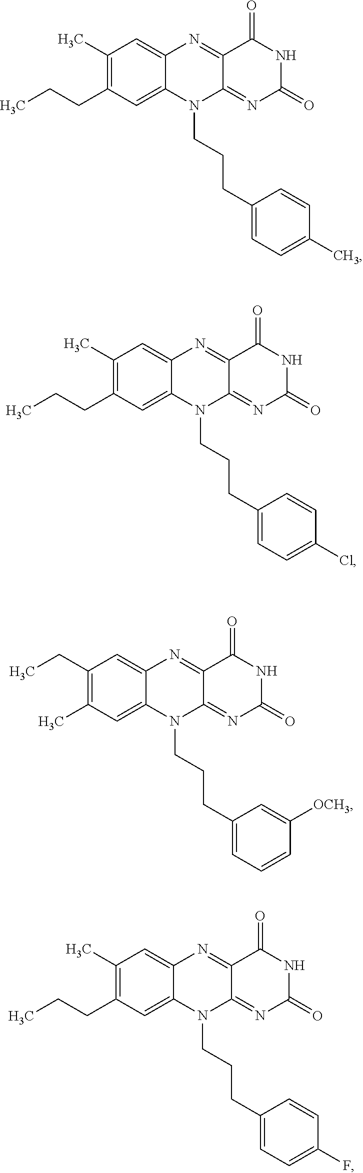

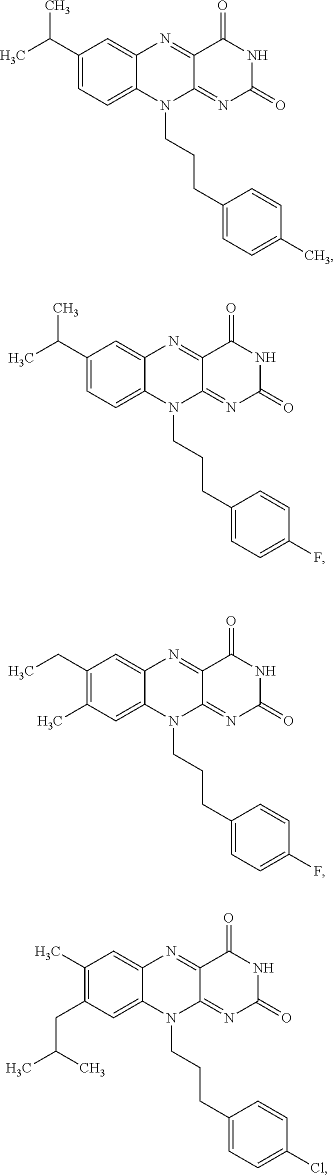 [SMILES: Cc1ccc(CCCn2c3nc(=O)[nH]c(=O)c-3nc3cc(C(C)C)ccc32)cc1 ; CC(C)c1ccc2c(c1)nc1c(=O)[nH]c(=O)nc-1n2CCCc1ccc(F)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C]

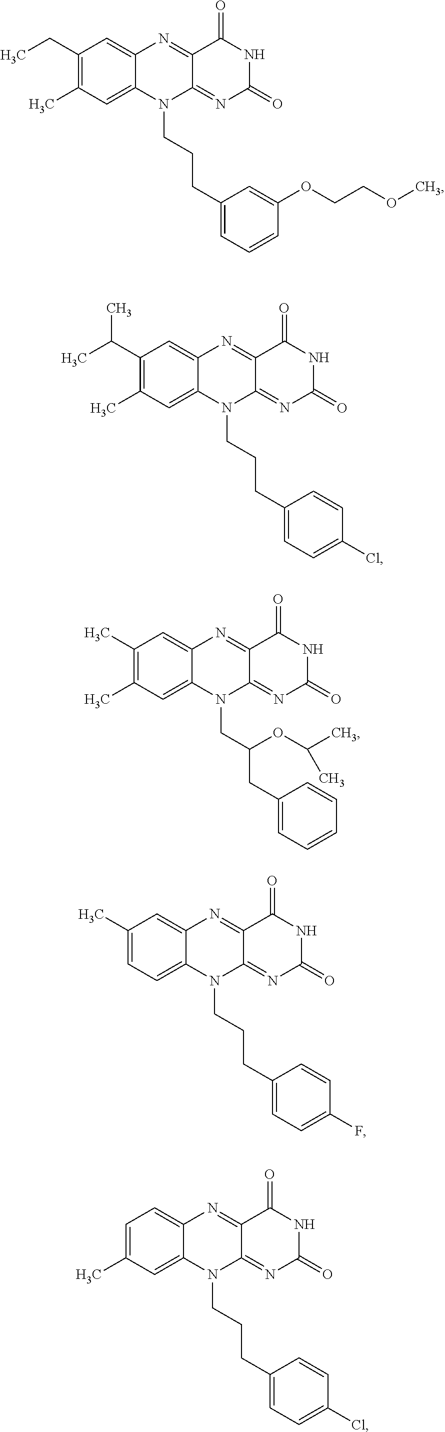 [SMILES: CCc1cc2nc3c(=O)[nH]c(=O)nc-3n(CCCc3cccc(OCCOC)c3)c2cc1C ; Cc1cc2c(cc1C(C)C)nc1c(=O)[nH]c(=O)nc-1n2CCCc1ccc(Cl)cc1 ; Cc1cc2nc3c(=O)[nH]c(=O)nc-3n(CC(Cc3ccccc3)OC(C)C)c2cc1C ; Cc1ccc2c(c1)nc1c(=O)[nH]c(=O)nc-1n2CCCc1ccc(F)cc1 ; Cc1ccc2nc3c(=O)[nH]c(=O)nc-3n(CCCc3ccc(Cl)cc3)c2c1]

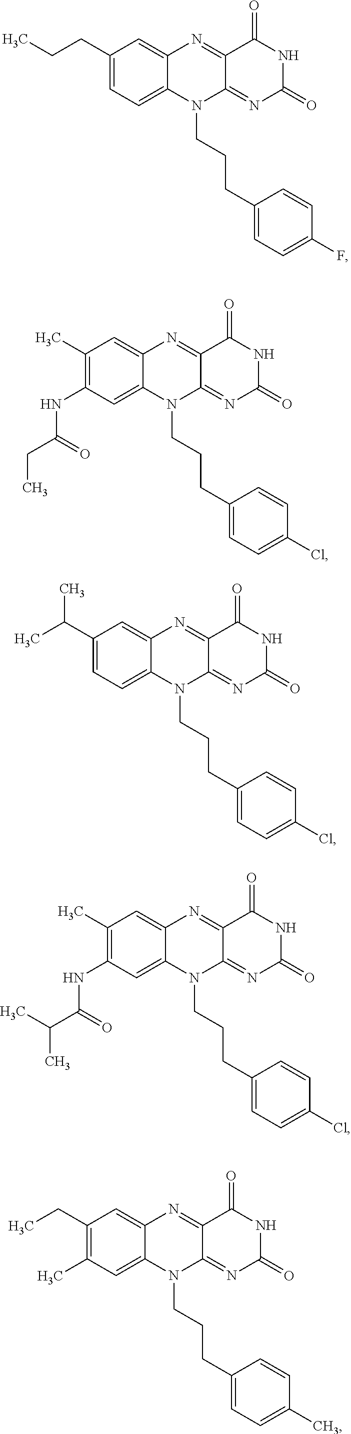

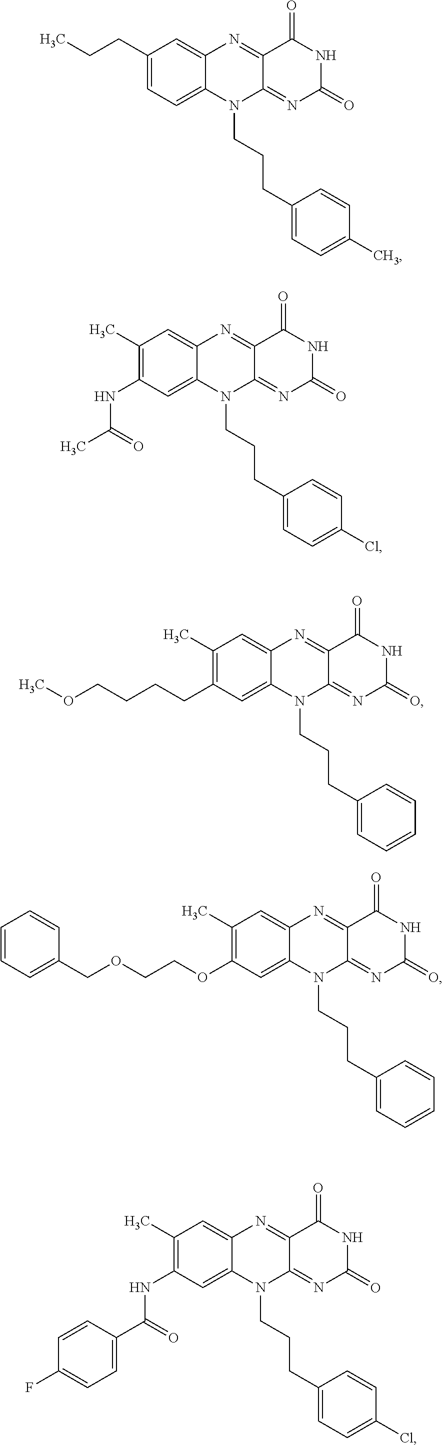

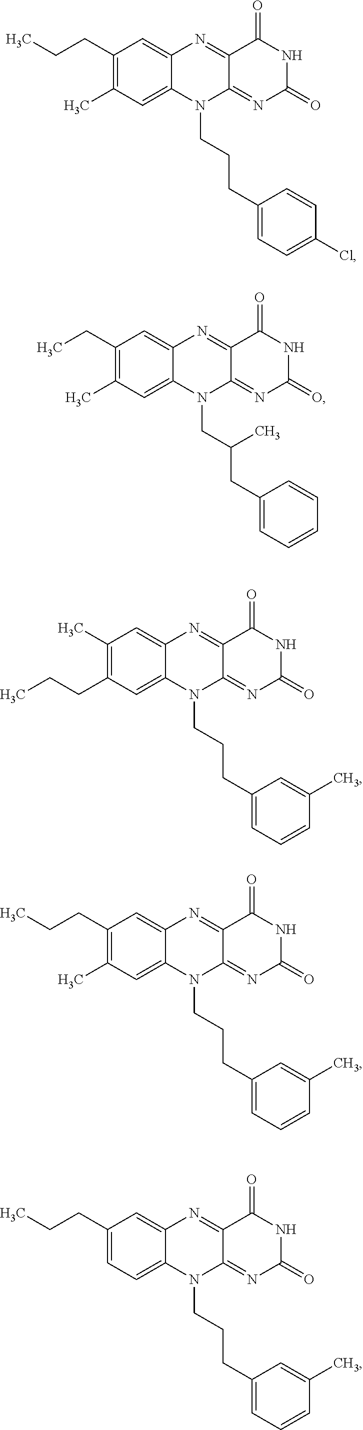

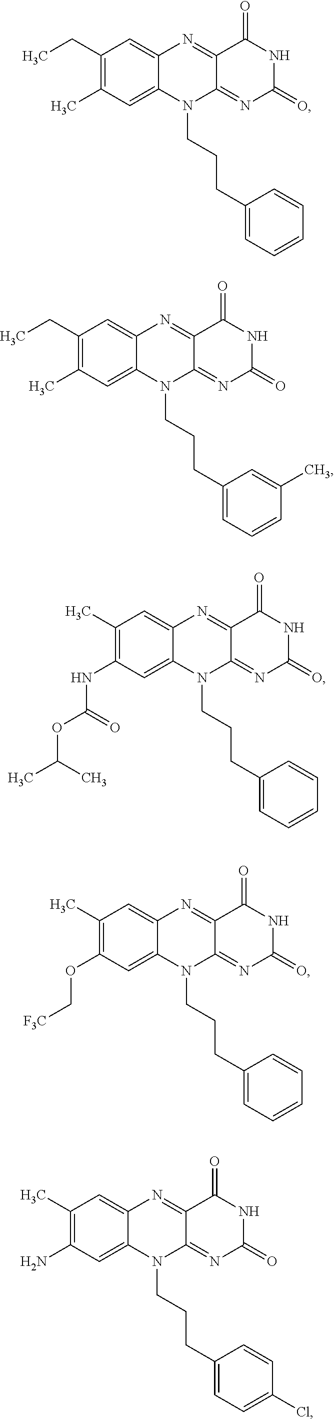

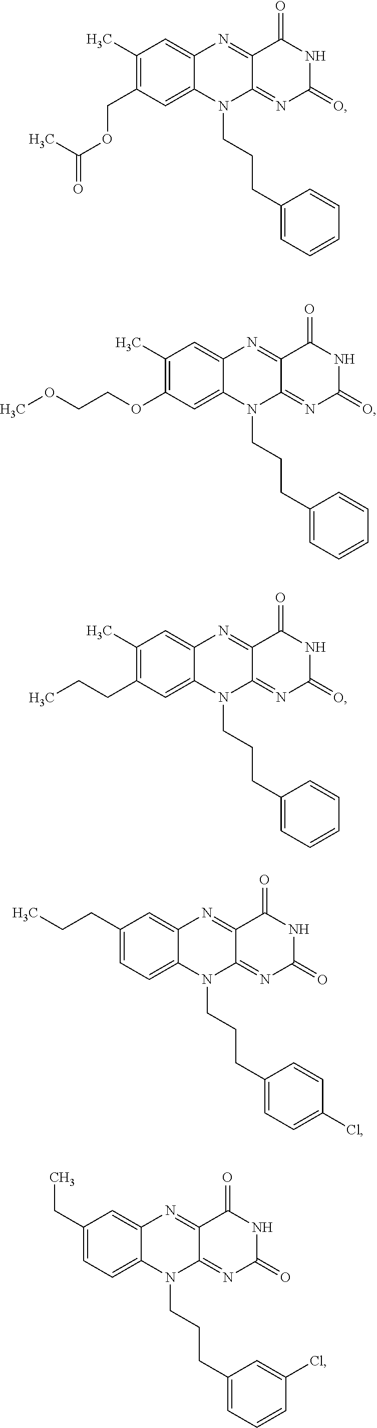 [SMILES: CC(=O)OCc1cc2c(cc1C)nc1c(=O)[nH]c(=O)nc-1n2CCCc1ccccc1 ; COCCOc1cc2c(cc1C)nc1c(=O)[nH]c(=O)nc-1n2CCCc1ccccc1 ; CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1 ; CCc1ccc2c(c1)nc1c(=O)[nH]c(=O)nc-1n2CCCc1cccc(Cl)c1]

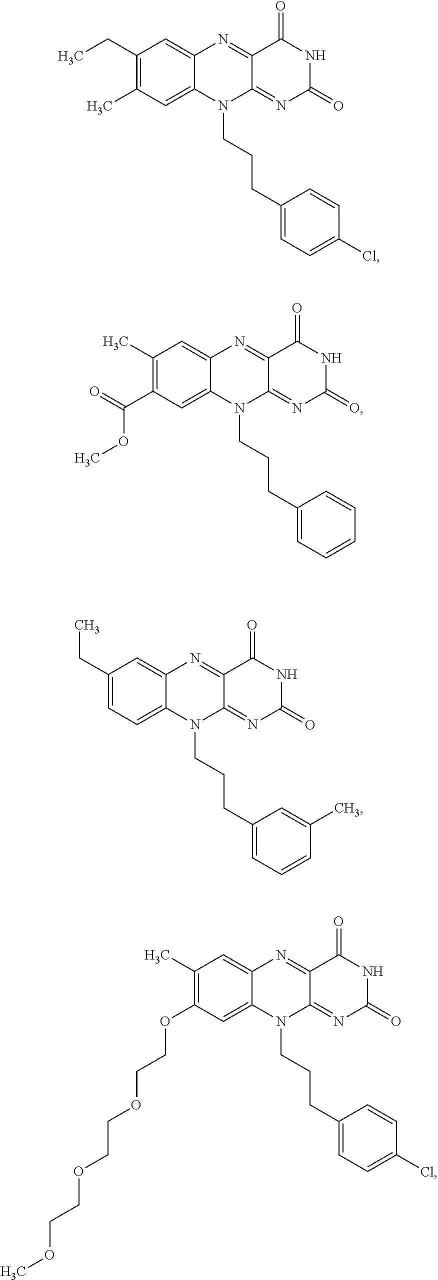 [SMILES: CCc1cc2nc3c(=O)[nH]c(=O)nc-3n(CCCc3ccc(Cl)cc3)c2cc1C ; COC(=O)c1cc2c(cc1C)nc1c(=O)[nH]c(=O)nc-1n2CCCc1ccccc1 ; CCc1ccc2c(c1)nc1c(=O)[nH]c(=O)nc-1n2CCCc1cccc(C)c1 ; COCCOCCOCCOc1cc2c(cc1C)nc1c(=O)[nH]c(=O)nc-1n2CCCc1ccc(Cl)cc1]

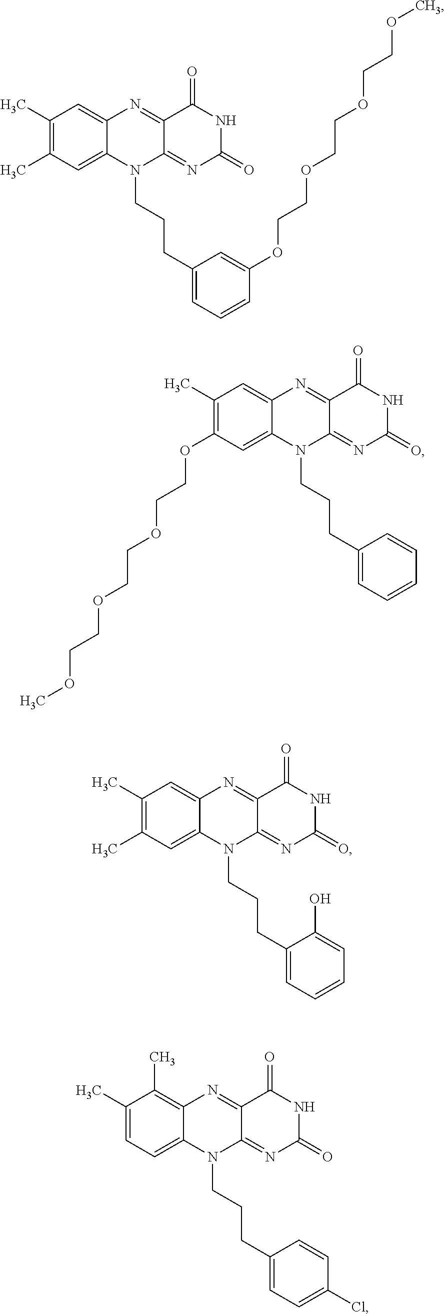 [SMILES: COCCOCCOCCOc1cccc(CCCn2c3nc(=O)[nH]c(=O)c-3nc3cc(C)c(C)cc32)c1 ; COCCOCCOCCOc1cc2c(cc1C)nc1c(=O)[nH]c(=O)nc-1n2CCCc1ccccc1 ; Cc1cc2nc3c(=O)[nH]c(=O)nc-3n(CCCc3ccccc3O)c2cc1C ; Cc1ccc2c(nc3c(=O)[nH]c(=O)nc-3n2CCCc2ccc(Cl)cc2)c1C]

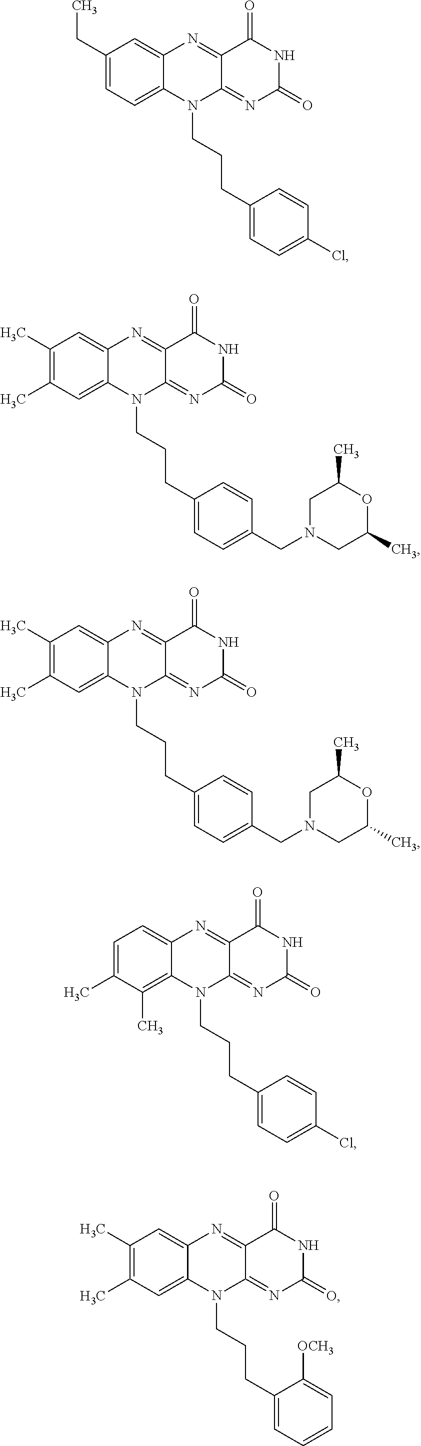 [SMILES: CCc1ccc2c(c1)nc1c(=O)[nH]c(=O)nc-1n2CCCc1ccc(Cl)cc1 ; Cc1cc2nc3c(=O)[nH]c(=O)nc-3n(CCCc3ccc(CN4C[C@@H](C)O[C@@H](C)C4)cc3)c2cc1C ; Cc1cc2nc3c(=O)[nH]c(=O)nc-3n(CCCc3ccc(CN4C[C@@H](C)O[C@H](C)C4)cc3)c2cc1C ; Cc1ccc2nc3c(=O)[nH]c(=O)nc-3n(CCCc3ccc(Cl)cc3)c2c1C ; COc1ccccc1CCCn1c2nc(=O)[nH]c(=O)c-2nc2cc(C)c(C)cc21]

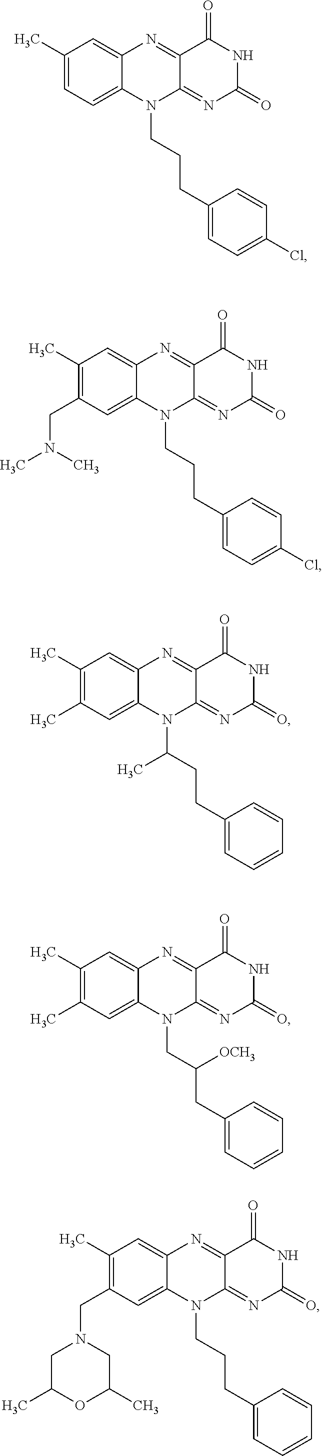

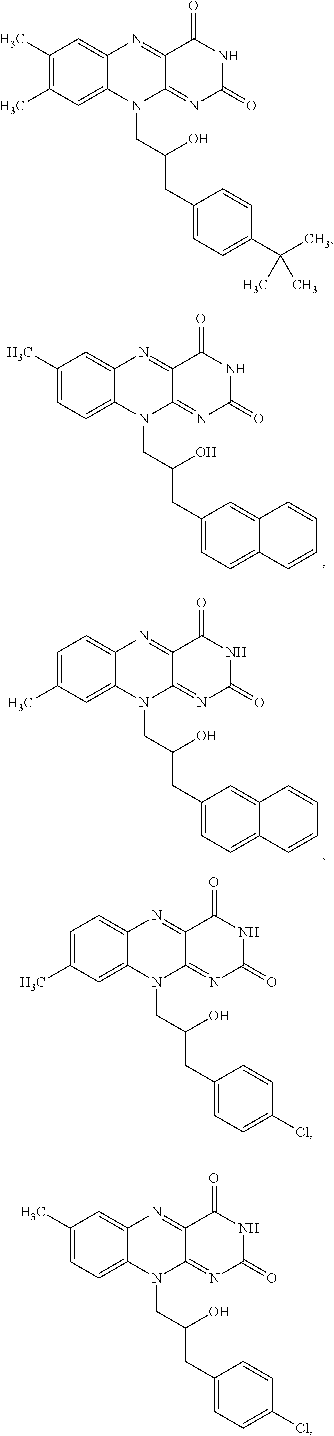

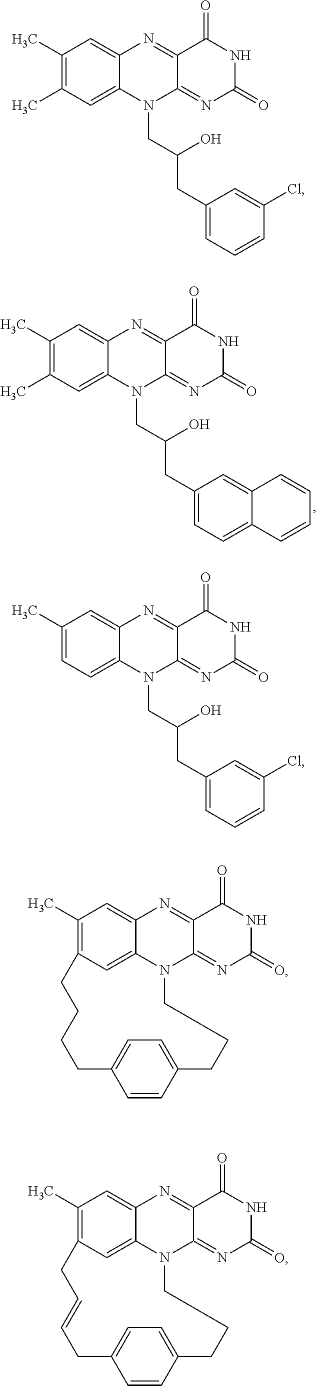

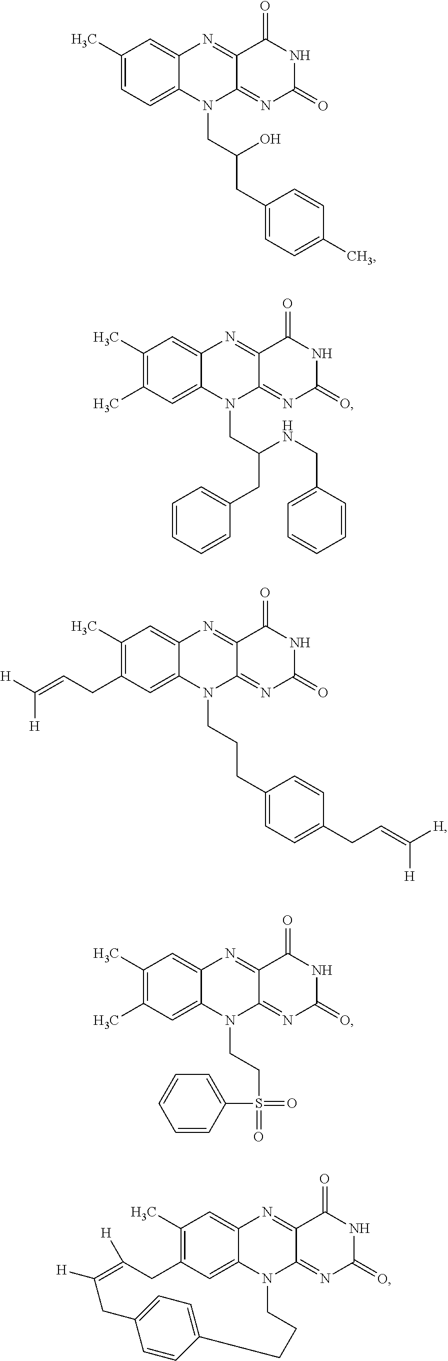

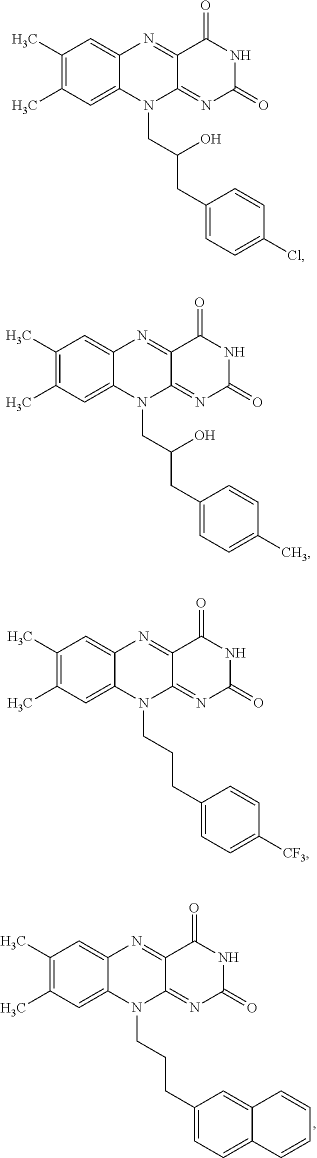 [SMILES: Cc1cc2nc3c(=O)[nH]c(=O)nc-3n(CC(O)Cc3ccc(Cl)cc3)c2cc1C ; Cc1ccc(CC(O)Cn2c3nc(=O)[nH]c(=O)c-3nc3cc(C)c(C)cc32)cc1 ; Cc1cc2nc3c(=O)[nH]c(=O)nc-3n(CCCc3ccc(C(F)(F)F)cc3)c2cc1C ; Cc1cc2nc3c(=O)[nH]c(=O)nc-3n(CCCc3ccc4ccccc4c3)c2cc1C]

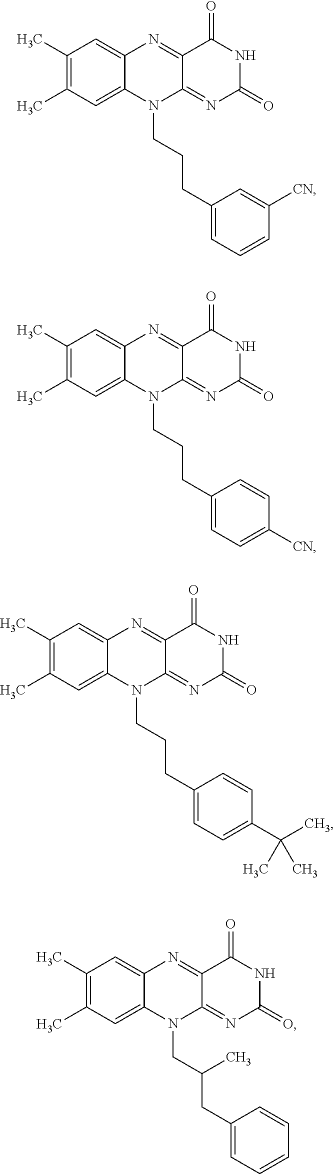 [SMILES: Cc1cc2nc3c(=O)[nH]c(=O)nc-3n(CCCc3cccc(C#N)c3)c2cc1C ; Cc1cc2nc3c(=O)[nH]c(=O)nc-3n(CCCc3ccc(C#N)cc3)c2cc1C ; Cc1cc2nc3c(=O)[nH]c(=O)nc-3n(CCCc3ccc(C(C)(C)C)cc3)c2cc1C ; Cc1cc2nc3c(=O)[nH]c(=O)nc-3n(CC(C)Cc3ccccc3)c2cc1C]

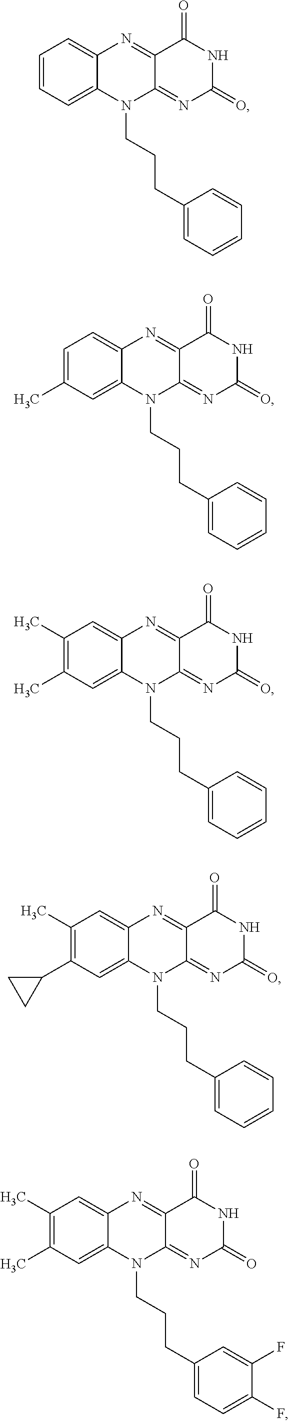

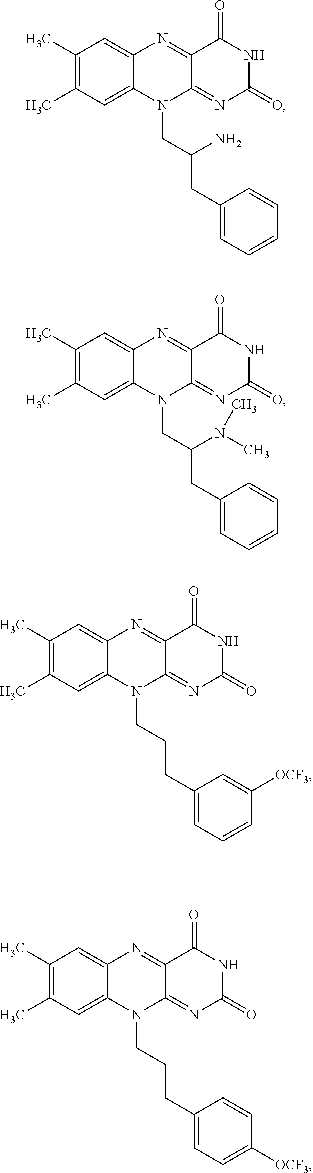 [SMILES: Cc1cc2nc3c(=O)[nH]c(=O)nc-3n(CC(N)Cc3ccccc3)c2cc1C ; Cc1cc2nc3c(=O)[nH]c(=O)nc-3n(CC(Cc3ccccc3)N(C)C)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C]

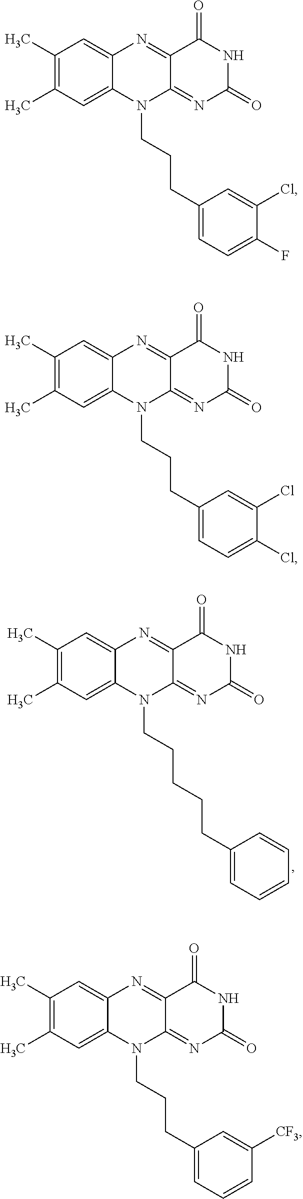 [SMILES: Cc1cc2nc3c(=O)[nH]c(=O)nc-3n(CCCc3ccc(F)c(Cl)c3)c2cc1C ; Cc1cc2nc3c(=O)[nH]c(=O)nc-3n(CCCc3ccc(Cl)c(Cl)c3)c2cc1C ; Cc1cc2nc3c(=O)[nH]c(=O)nc-3n(CCCCCc3ccccc3)c2cc1C ; Cc1cc2nc3c(=O)[nH]c(=O)nc-3n(CCCc3cccc(C(F)(F)F)c3)c2cc1C]

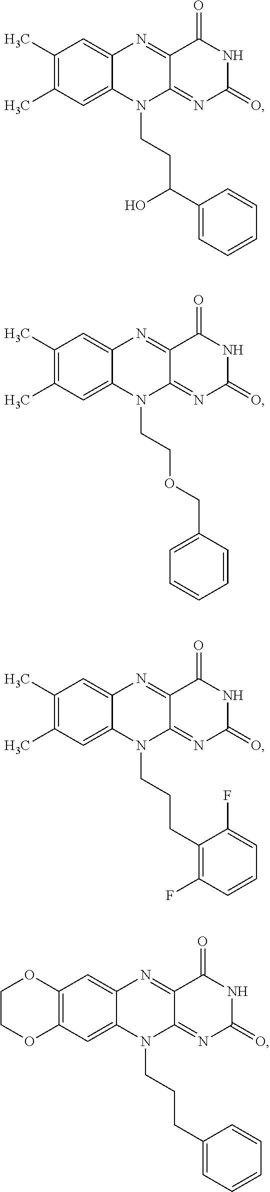

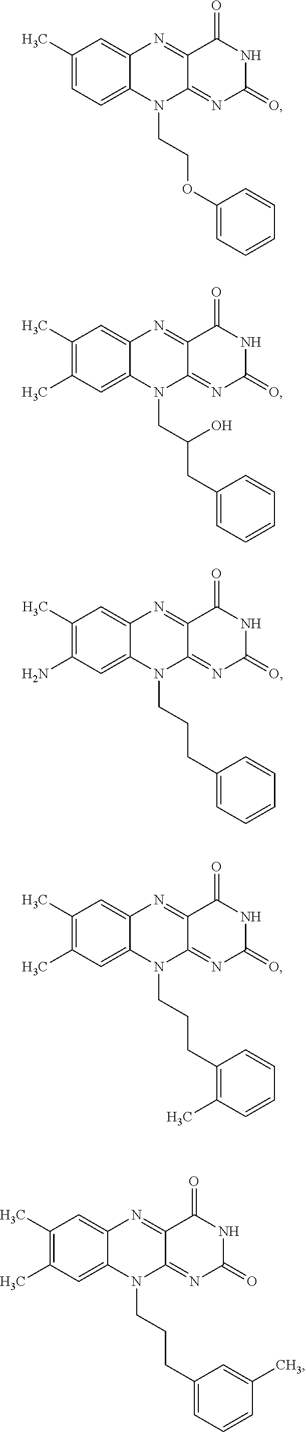

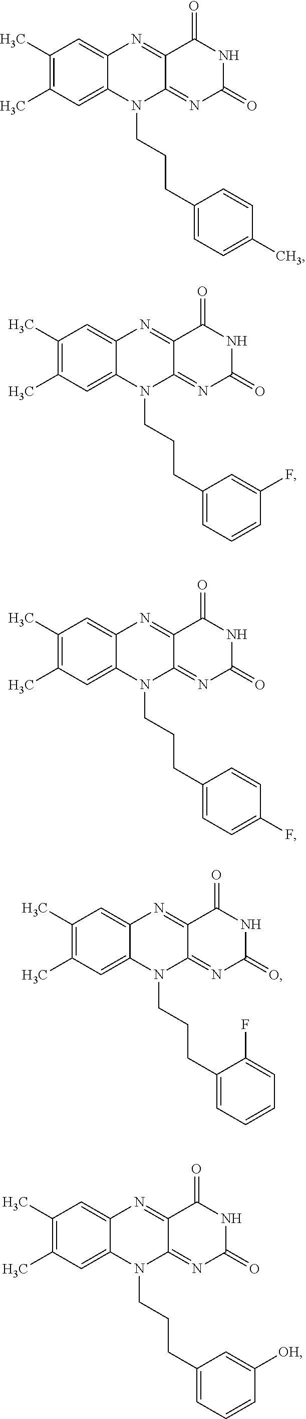

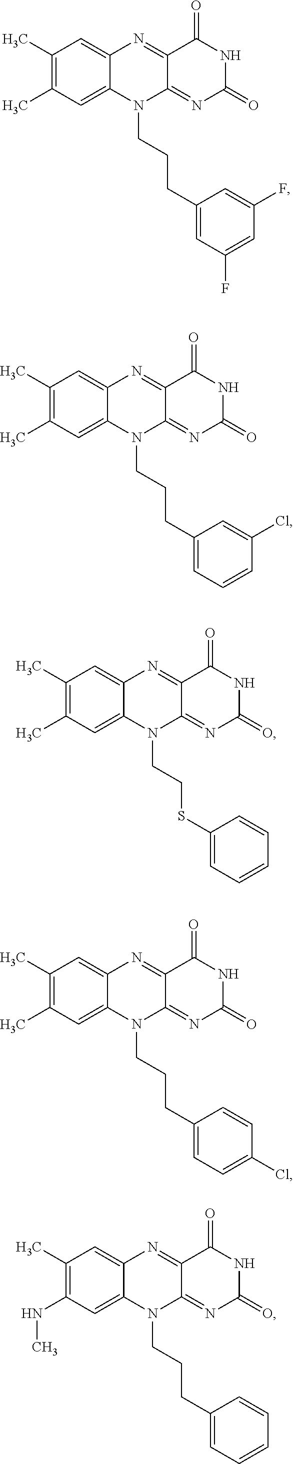

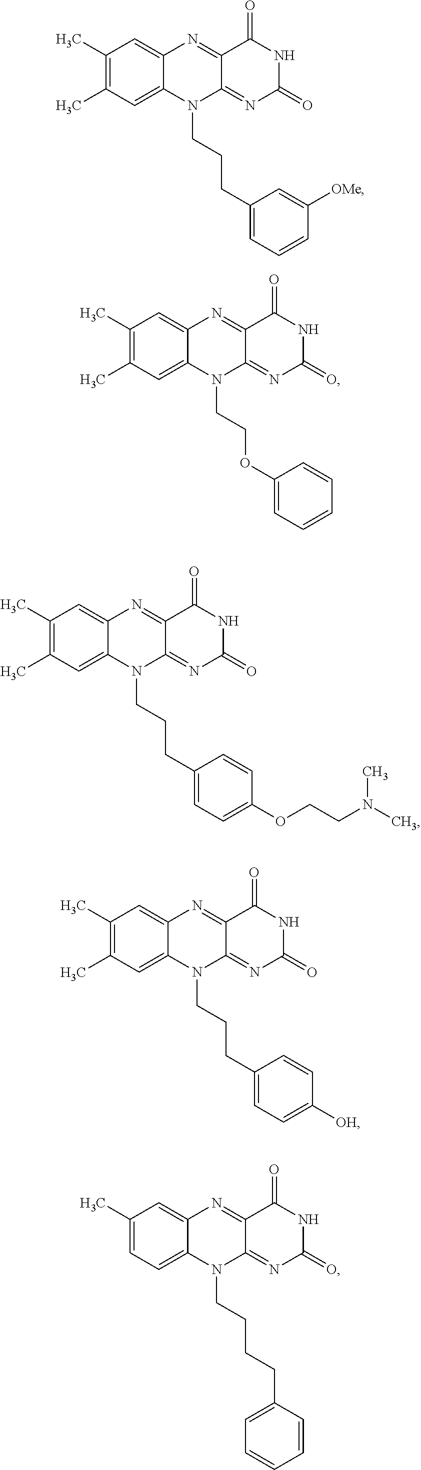

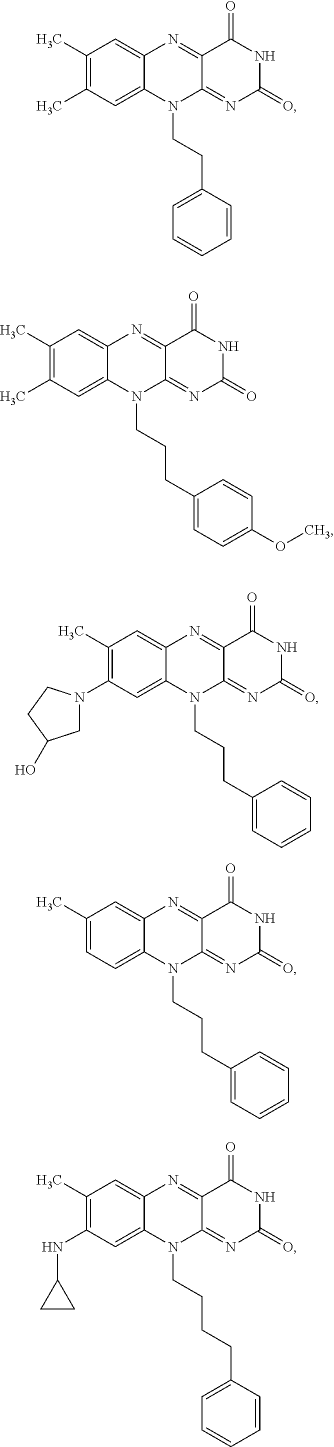

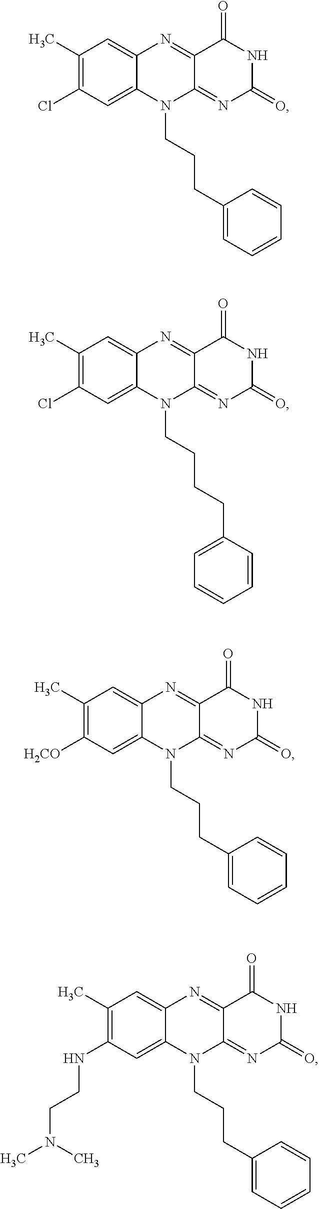

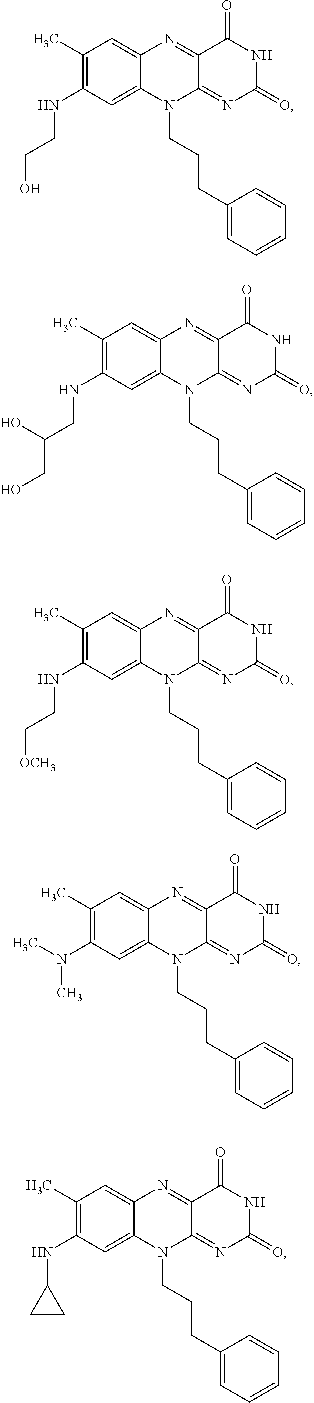

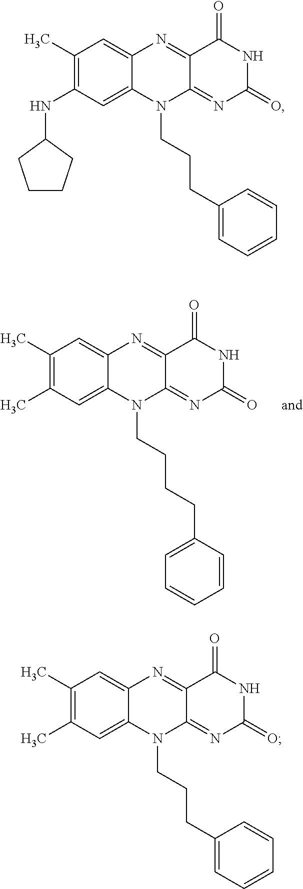

1.28. The compound according to any of the preceding formulae wherein said compound is selected from:

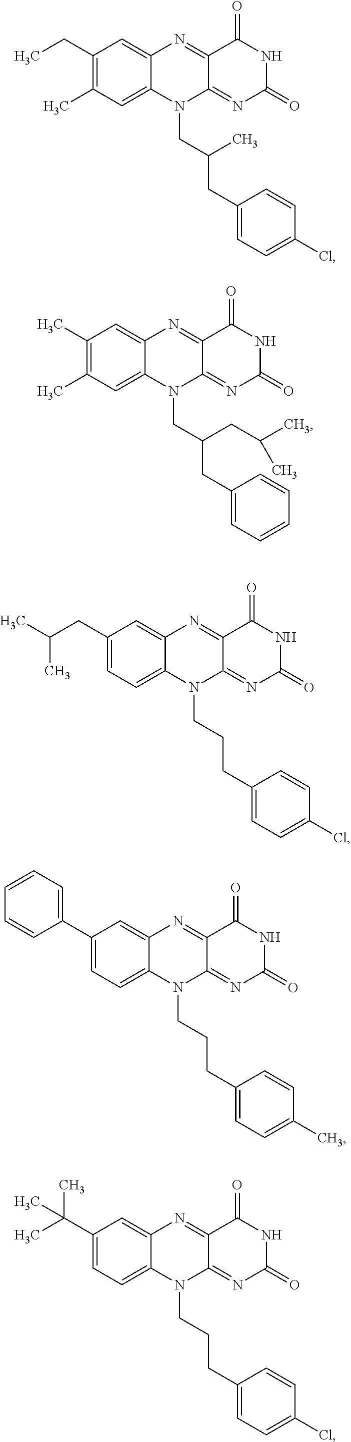 [SMILES: CCc1cc2nc3c(=O)[nH]c(=O)nc-3n(CC(C)Cc3ccc(Cl)cc3)c2cc1C ; Cc1cc2nc3c(=O)[nH]c(=O)nc-3n(CC(Cc3ccccc3)CC(C)C)c2cc1C ; CC(C)Cc1ccc2c(c1)nc1c(=O)[nH]c(=O)nc-1n2CCCc1ccc(Cl)cc1 ; Cc1ccc(CCCn2c3nc(=O)[nH]c(=O)c-3nc3cc(-c4ccccc4)ccc32)cc1 ; CC(C)(C)c1ccc2c(c1)nc1c(=O)[nH]c(=O)nc-1n2CCCc1ccc(Cl)cc1]

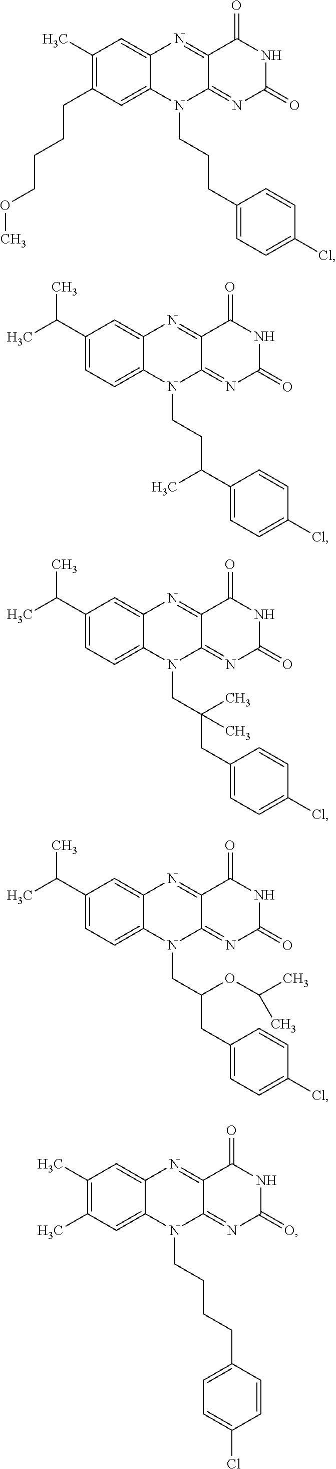

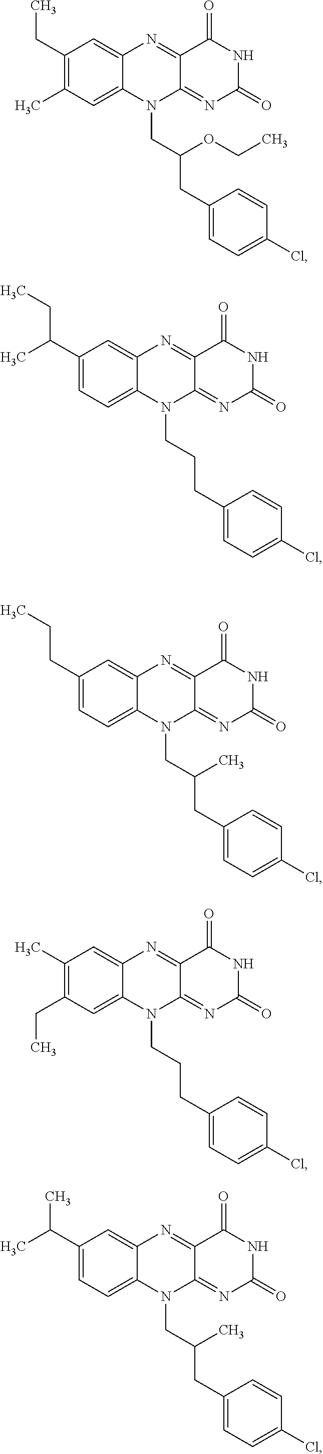

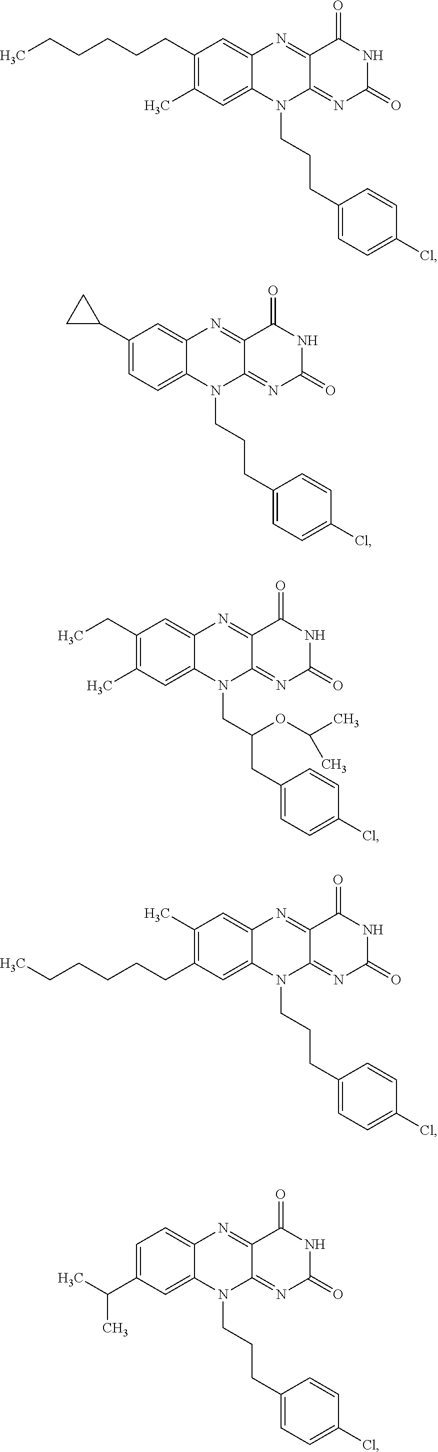 [SMILES: CCCCCCc1cc2nc3c(=O)[nH]c(=O)nc-3n(CCCc3ccc(Cl)cc3)c2cc1C ; O=c1nc2n(CCCc3ccc(Cl)cc3)c3ccc(C4CC4)cc3nc-2c(=O)[nH]1 ; CCc1cc2nc3c(=O)[nH]c(=O)nc-3n(CC(Cc3ccc(Cl)cc3)OC(C)C)c2cc1C ; CCCCCCc1cc2c(cc1C)nc1c(=O)[nH]c(=O)nc-1n2CCCc1ccc(Cl)cc1 ; CC(C)c1ccc2nc3c(=O)[nH]c(=O)nc-3n(CCCc3ccc(Cl)cc3)c2c1]

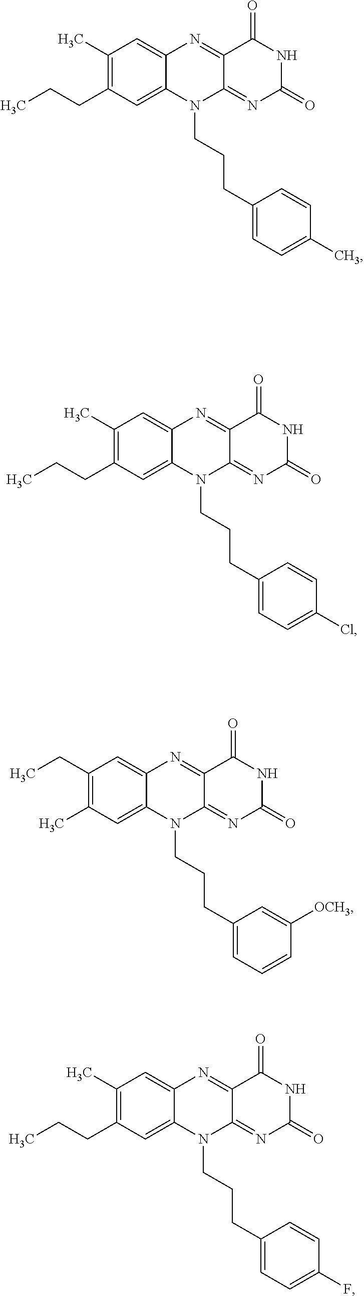 [SMILES: CCCc1cc2c(cc1C)nc1c(=O)[nH]c(=O)nc-1n2CCCc1ccc(C)cc1 ; CCCc1cc2c(cc1C)nc1c(=O)[nH]c(=O)nc-1n2CCCc1ccc(Cl)cc1 ; CCc1cc2nc3c(=O)[nH]c(=O)nc-3n(CCCc3cccc(OC)c3)c2cc1C ; CCCc1cc2c(cc1C)nc1c(=O)[nH]c(=O)nc-1n2CCCc1ccc(F)cc1]

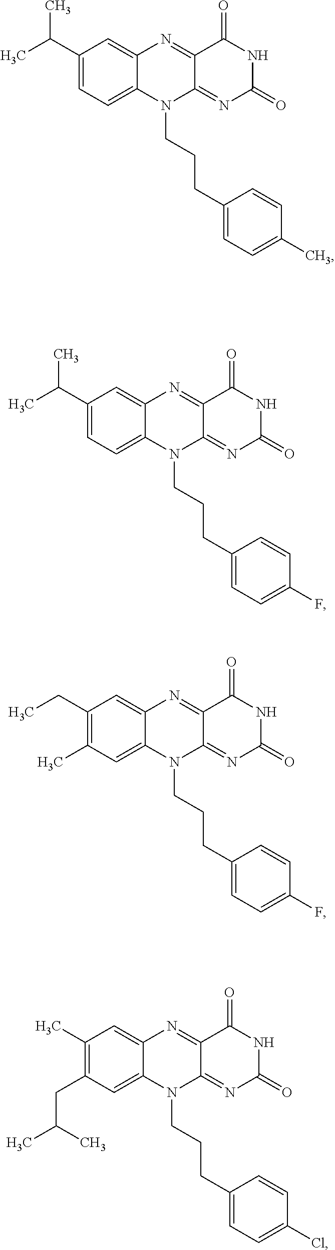 [SMILES: Cc1ccc(CCCn2c3nc(=O)[nH]c(=O)c-3nc3cc(C(C)C)ccc32)cc1 ; CC(C)c1ccc2c(c1)nc1c(=O)[nH]c(=O)nc-1n2CCCc1ccc(F)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C]

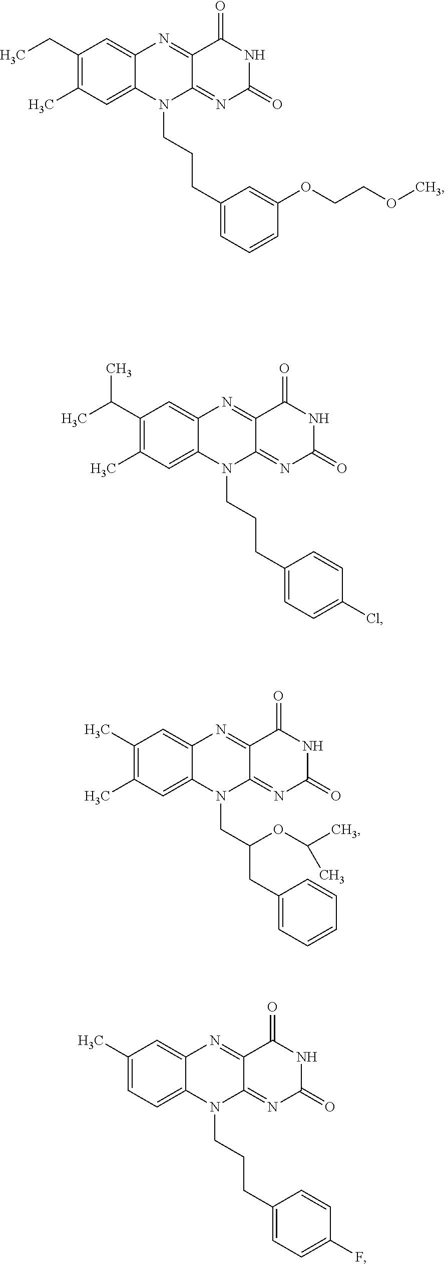 [SMILES: CCc1cc2nc3c(=O)[nH]c(=O)nc-3n(CCCc3cccc(OCCOC)c3)c2cc1C ; Cc1cc2c(cc1C(C)C)nc1c(=O)[nH]c(=O)nc-1n2CCCc1ccc(Cl)cc1 ; Cc1cc2nc3c(=O)[nH]c(=O)nc-3n(CC(Cc3ccccc3)OC(C)C)c2cc1C ; Cc1ccc2c(c1)nc1c(=O)[nH]c(=O)nc-1n2CCCc1ccc(F)cc1]

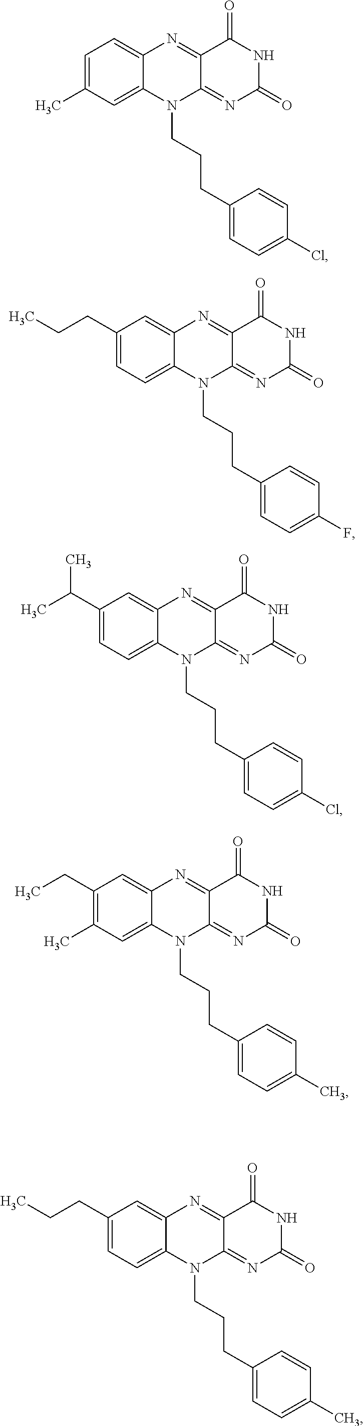

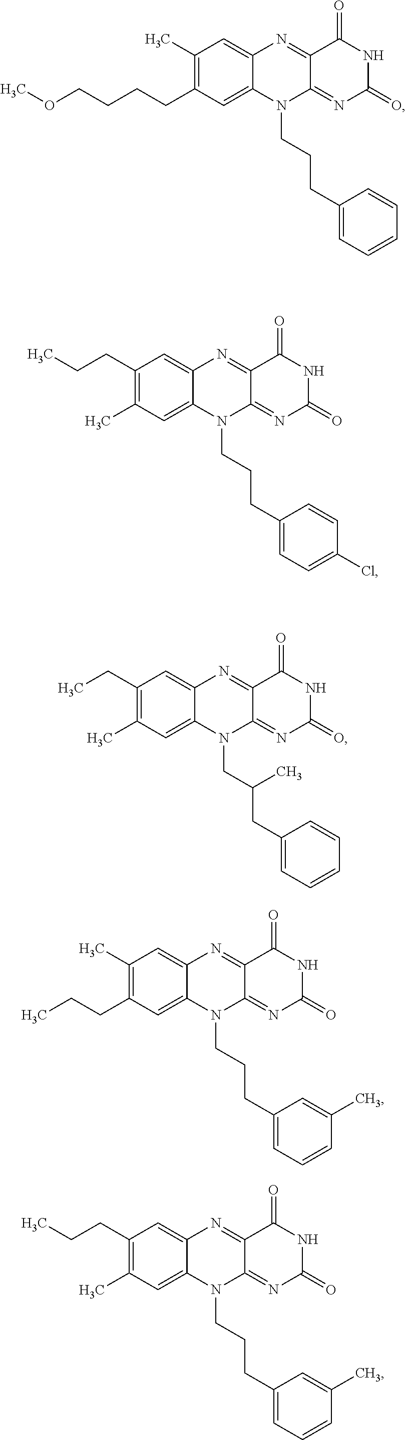

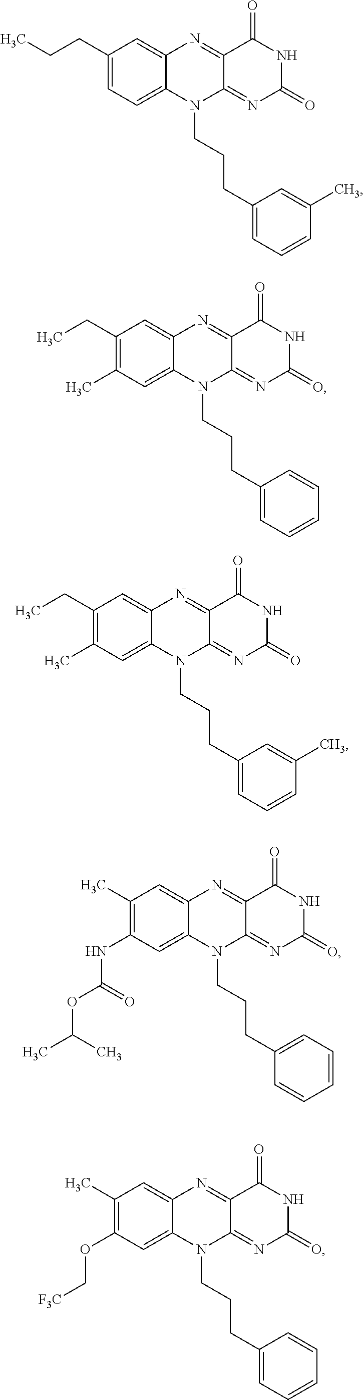

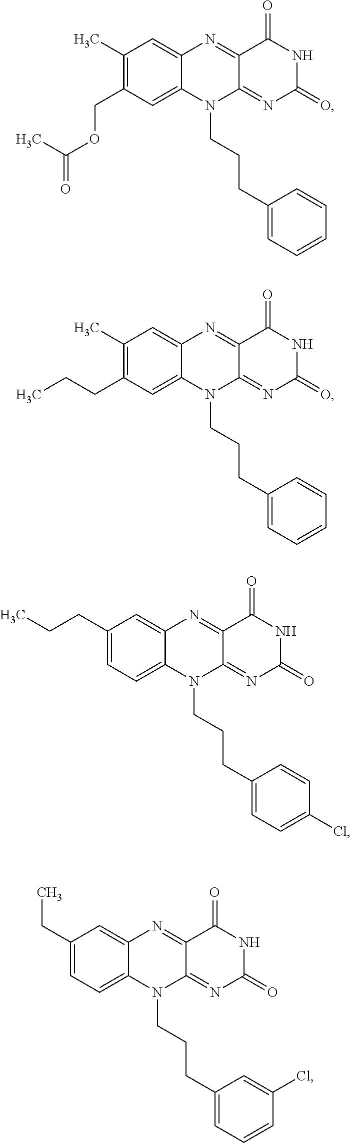 [SMILES: CC(=O)OCc1cc2c(cc1C)nc1c(=O)[nH]c(=O)nc-1n2CCCc1ccccc1 ; CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1 ; CCc1ccc2c(c1)nc1c(=O)[nH]c(=O)nc-1n2CCCc1cccc(Cl)c1]

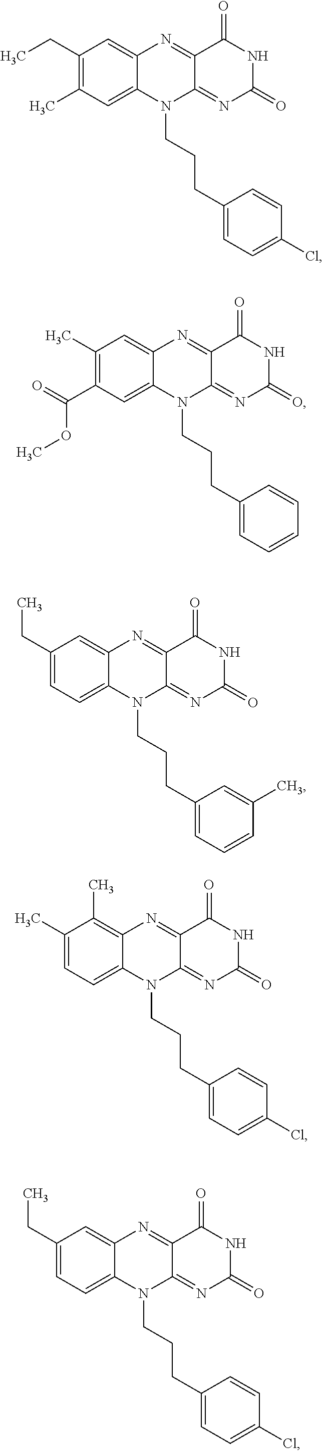

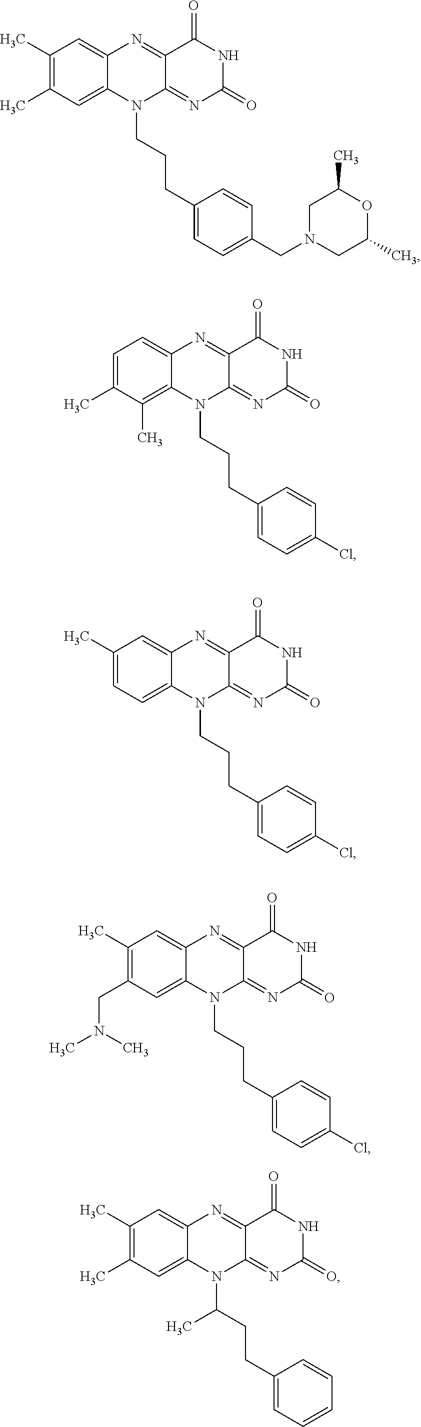

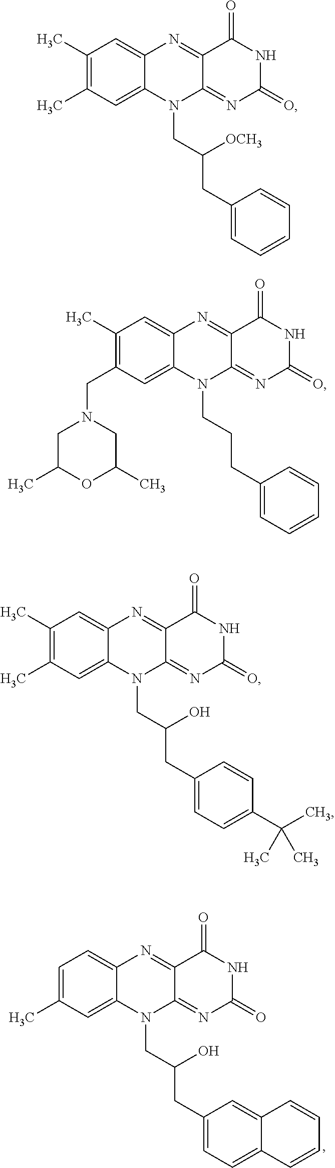

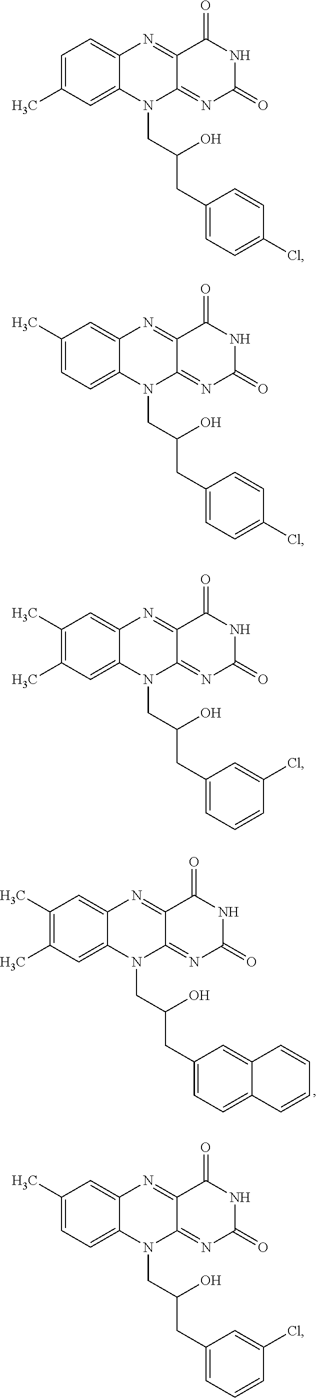

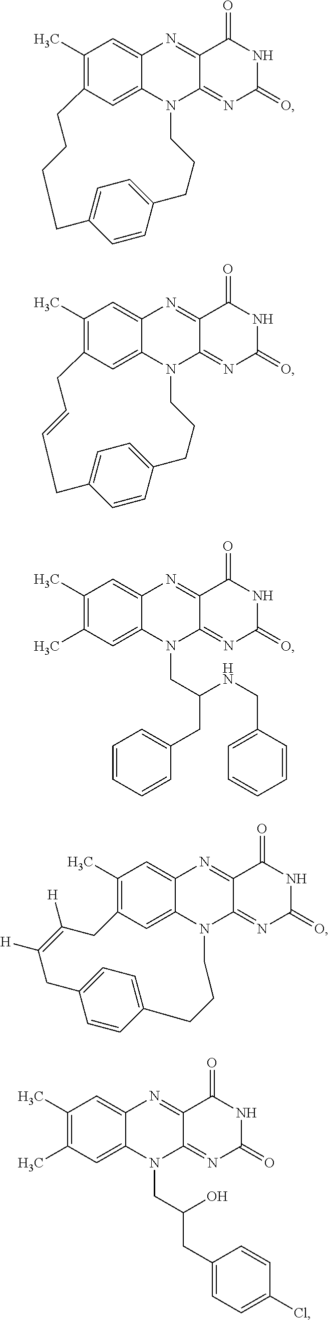

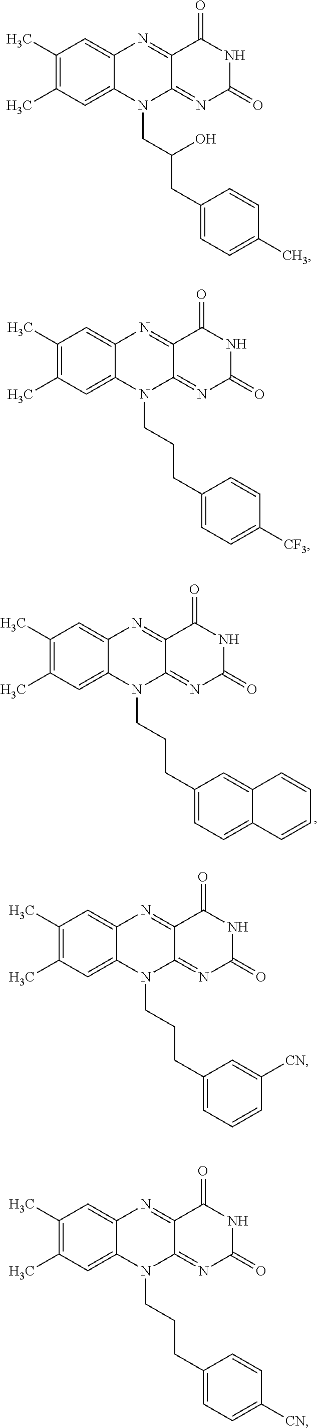

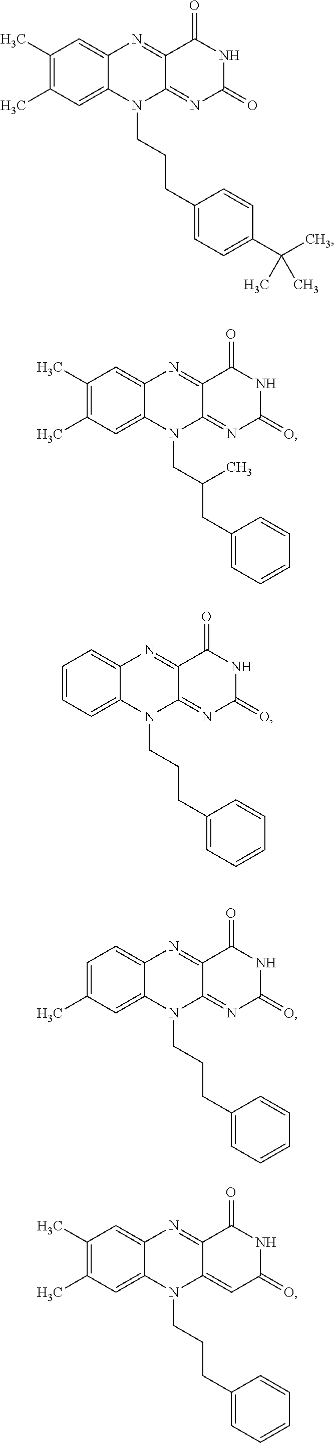

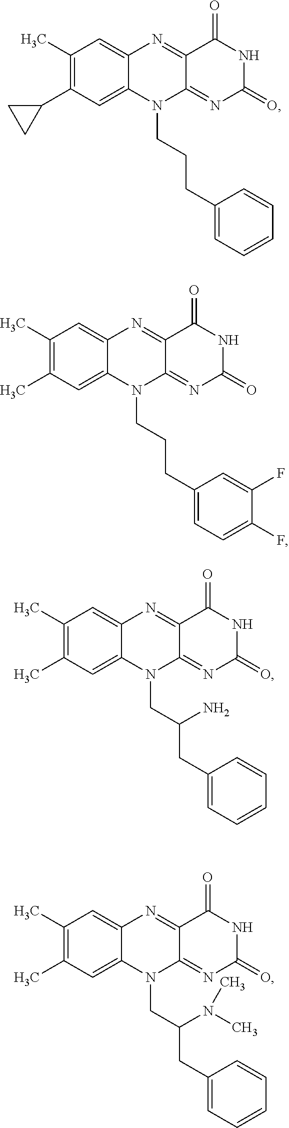

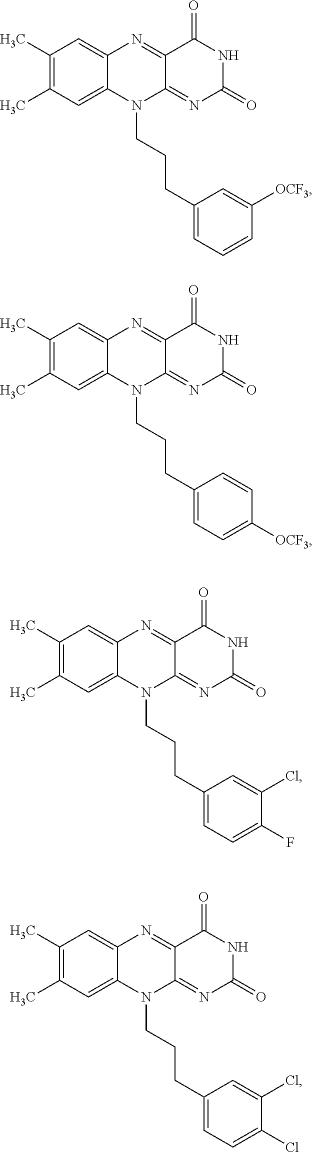

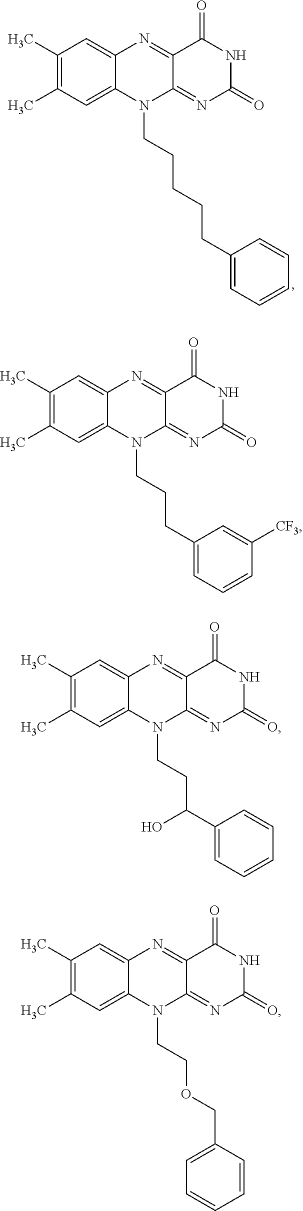

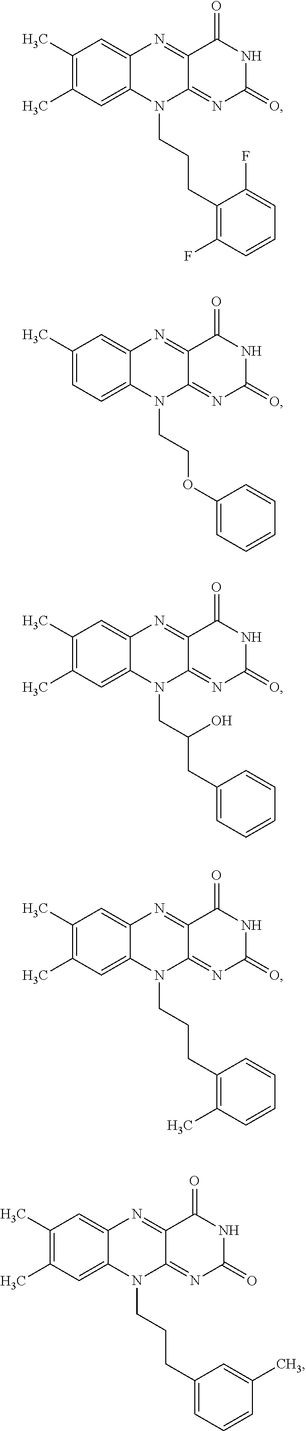

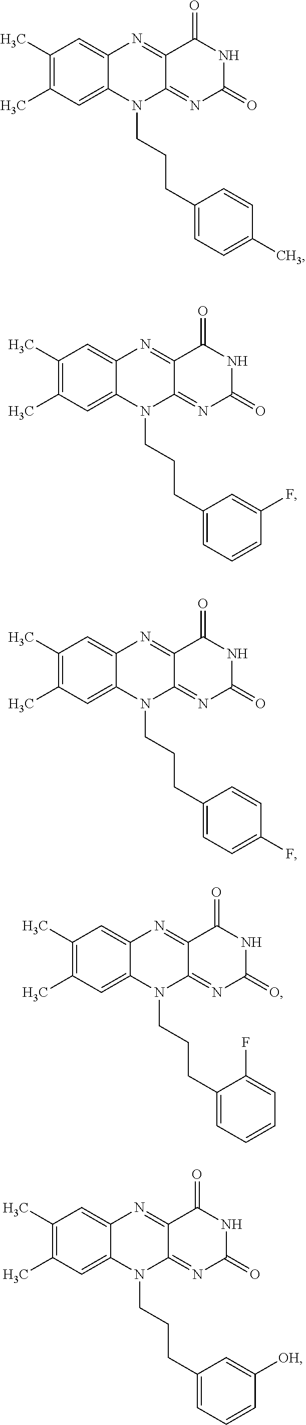

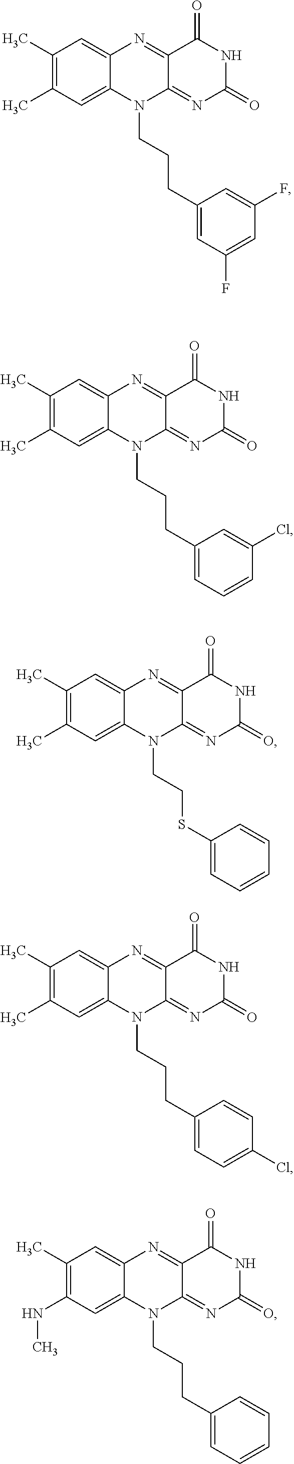

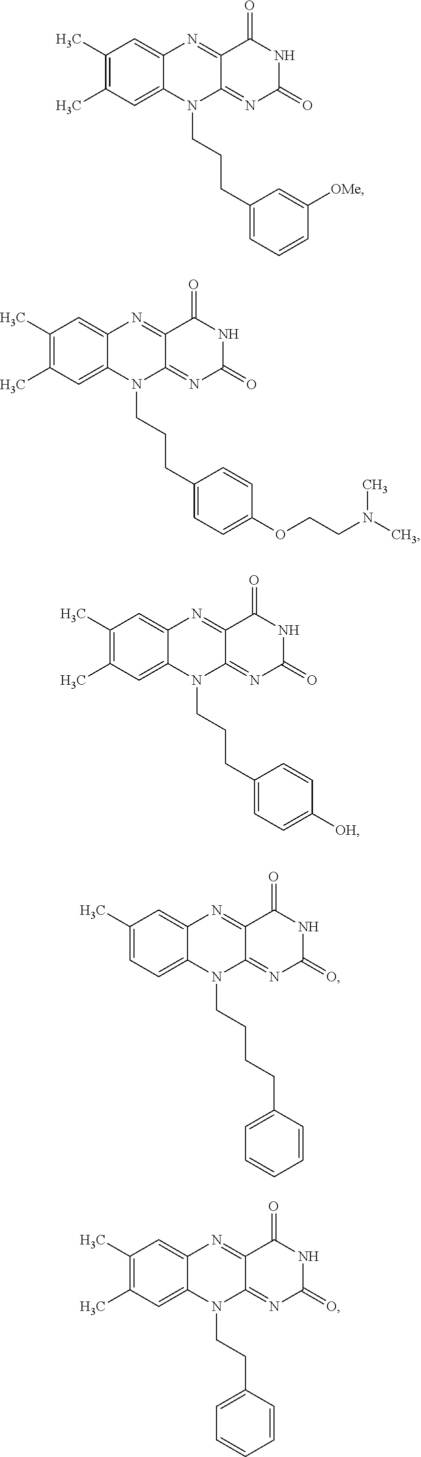

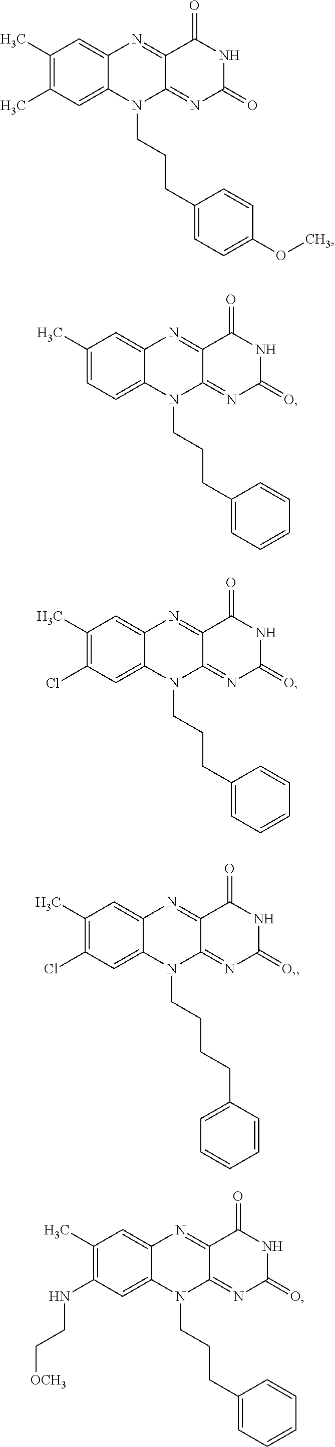

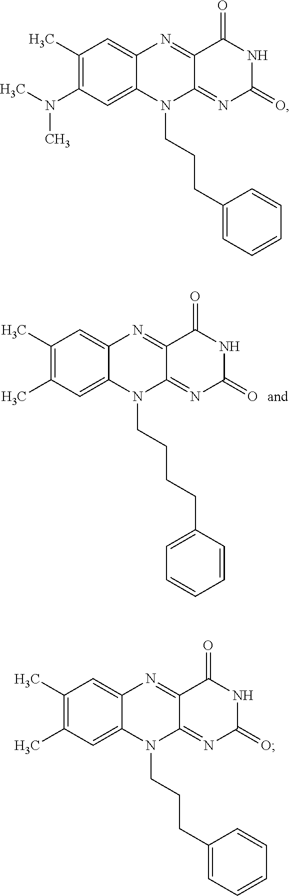 [SMILES: Cc1cc2nc3c(=O)[nH]c(=O)nc-3n(CCCc3ccccc3)c2cc1N(C)C ; Cc1cc2nc3c(=O)[nH]c(=O)nc-3n(CCCCc3ccccc3)c2cc1C ; Cc1cc2nc3c(=O)[nH]c(=O)nc-3n(CCCc3ccccc3)c2cc1C]

1.29. The compound according to any of the preceding formulae wherein said compound is selected from:

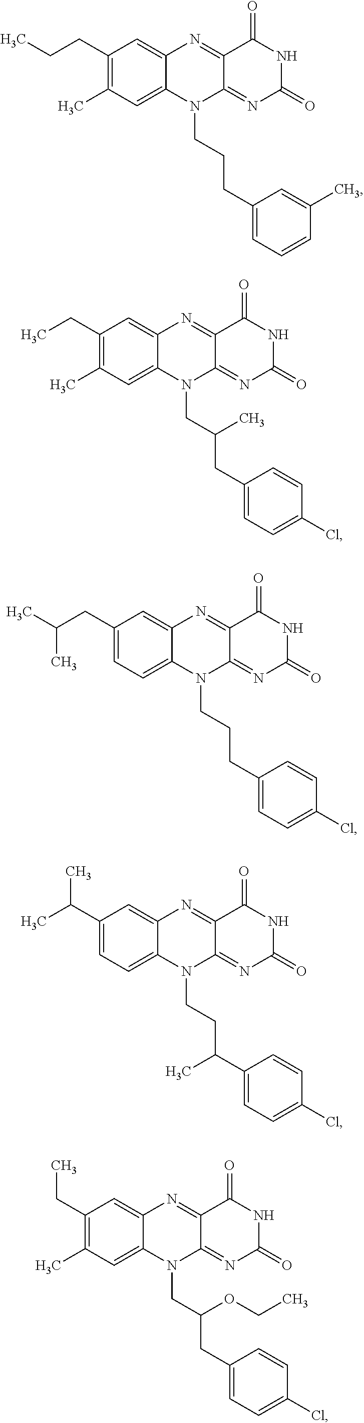

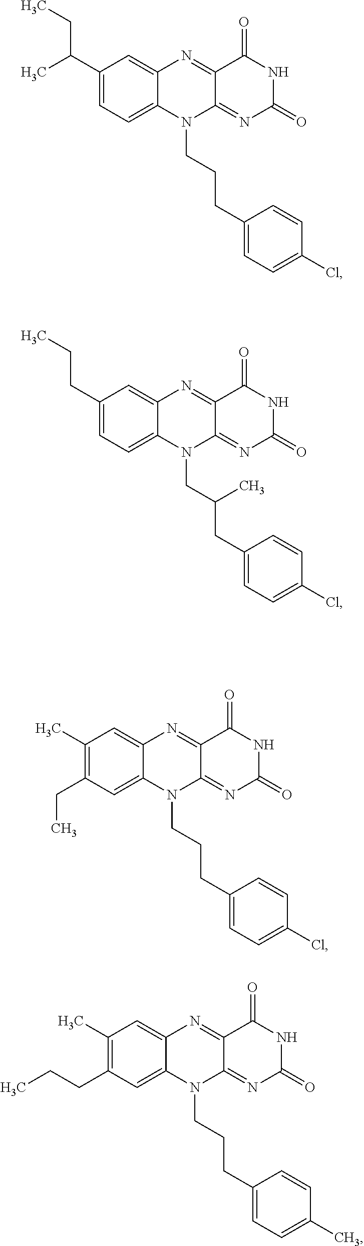

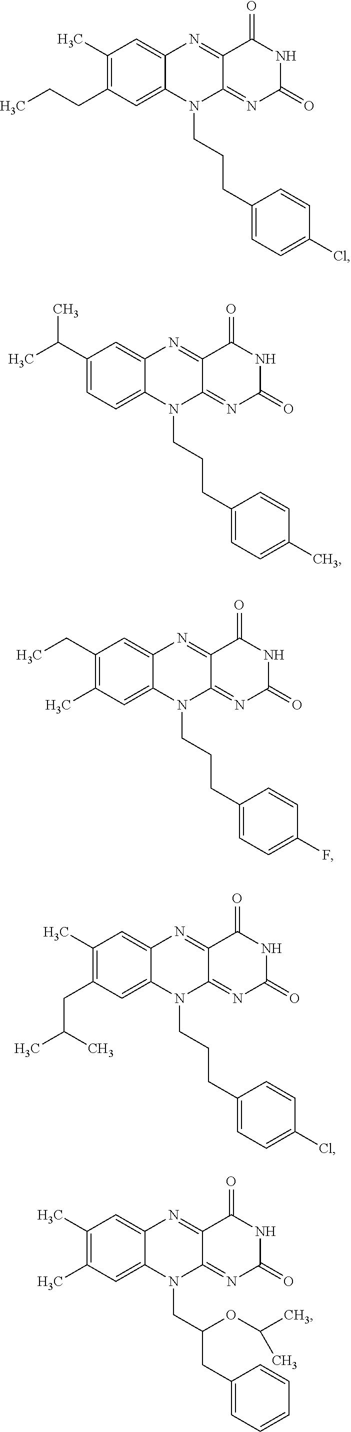 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C ; Cc1cc2nc3c(=O)[nH]c(=O)nc-3n(CC(Cc3ccccc3)OC(C)C)c2cc1C]

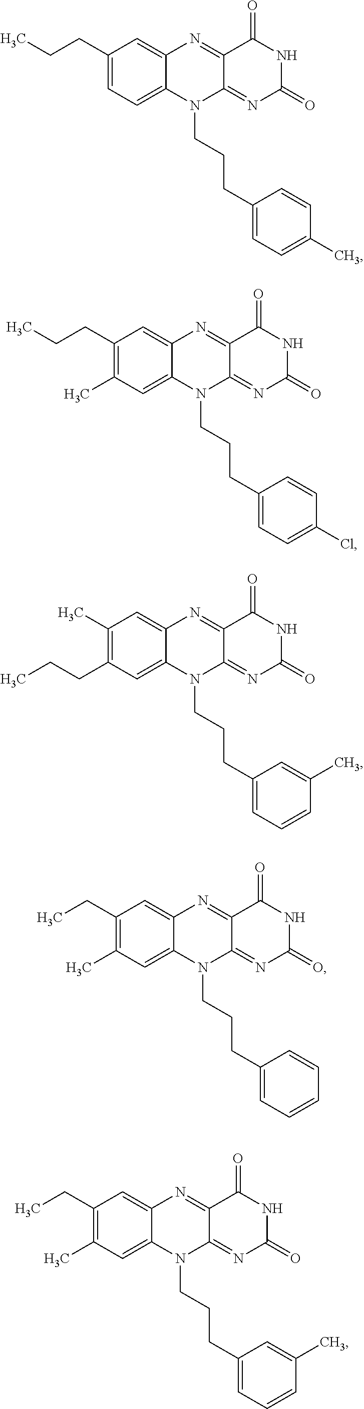

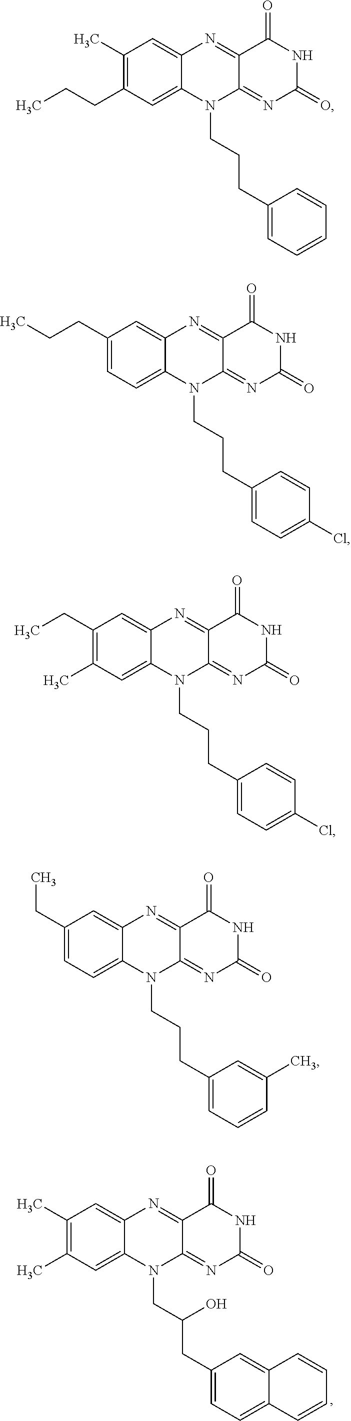 [SMILES: CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1 ; CCc1cc2nc3c(=O)[nH]c(=O)nc-3n(CCCc3ccc(Cl)cc3)c2cc1C ; CCc1ccc2c(c1)nc1c(=O)[nH]c(=O)nc-1n2CCCc1cccc(C)c1 ; Cc1cc2nc3c(=O)[nH]c(=O)nc-3n(CC(O)Cc3ccc4ccccc4c3)c2cc1C]

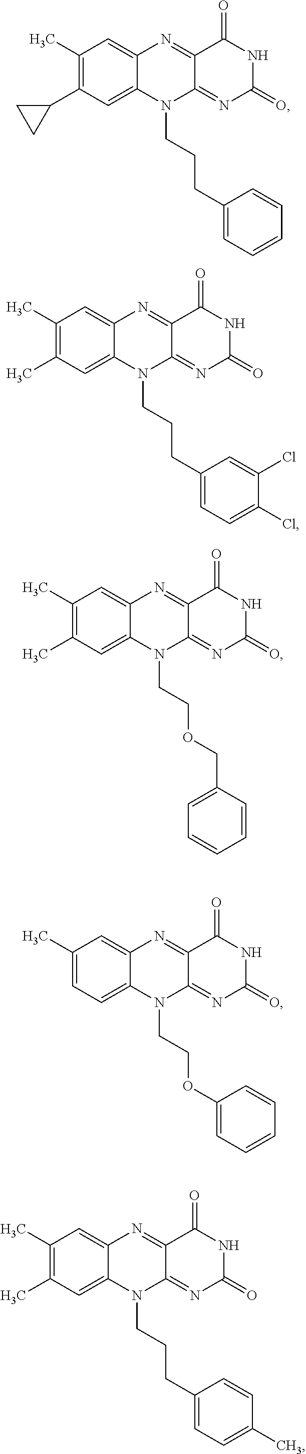

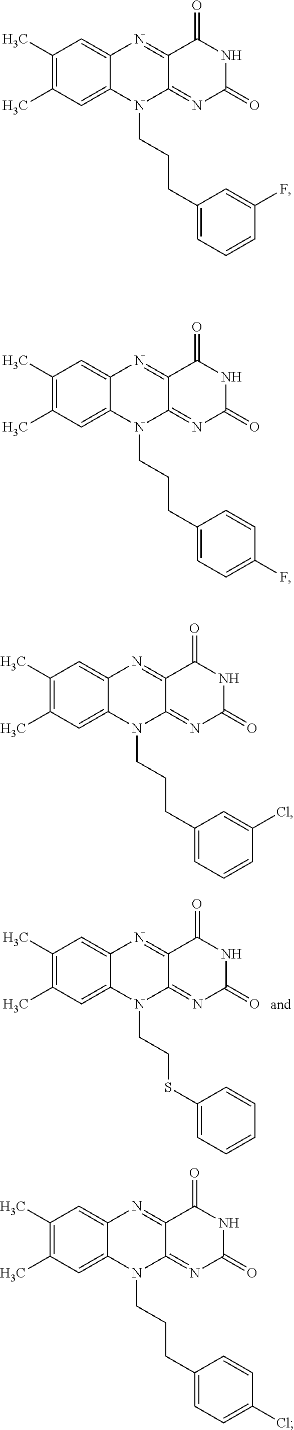

1.30. The compound according to any of the preceding formulae wherein said compound is selected from:

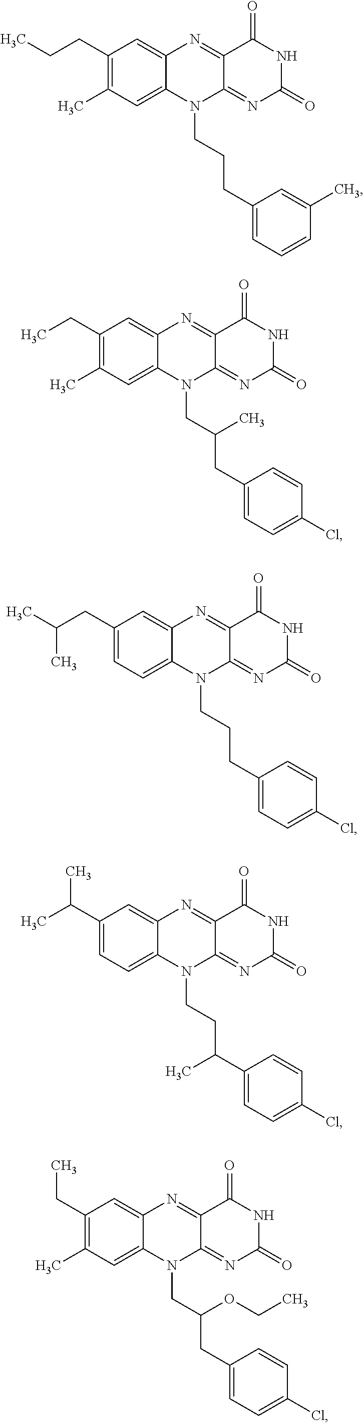

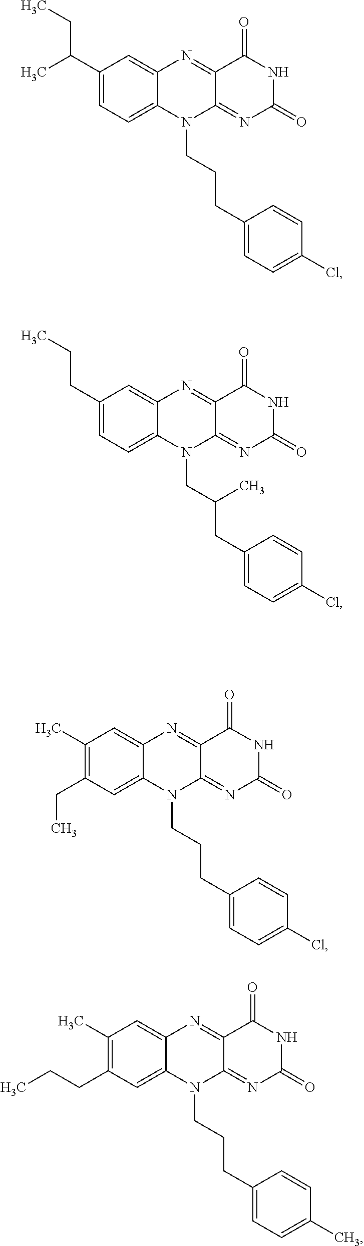

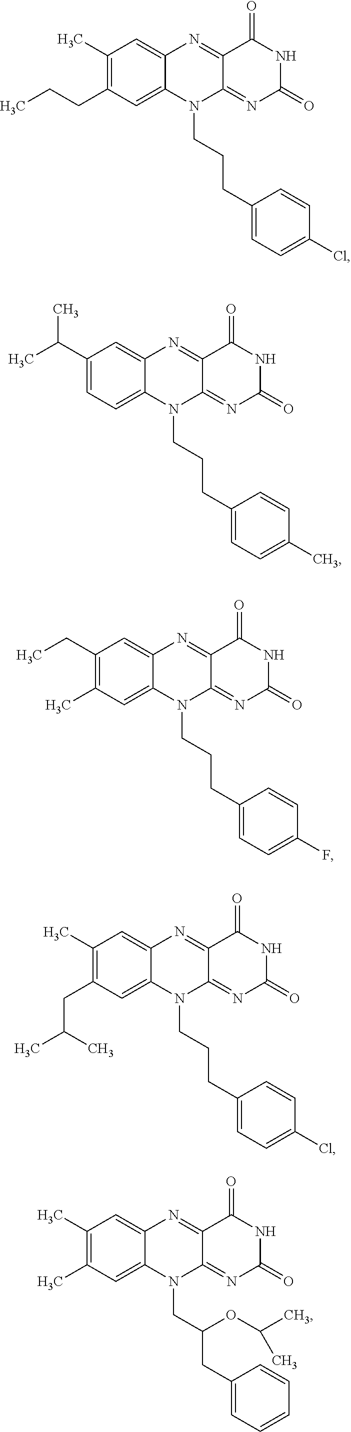 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C ; Cc1cc2nc3c(=O)[nH]c(=O)nc-3n(CC(Cc3ccccc3)OC(C)C)c2cc1C]

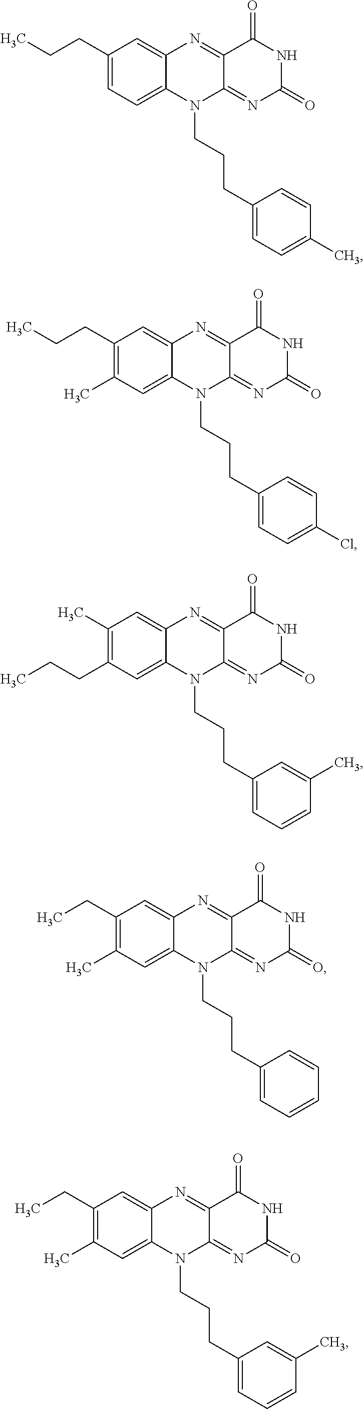

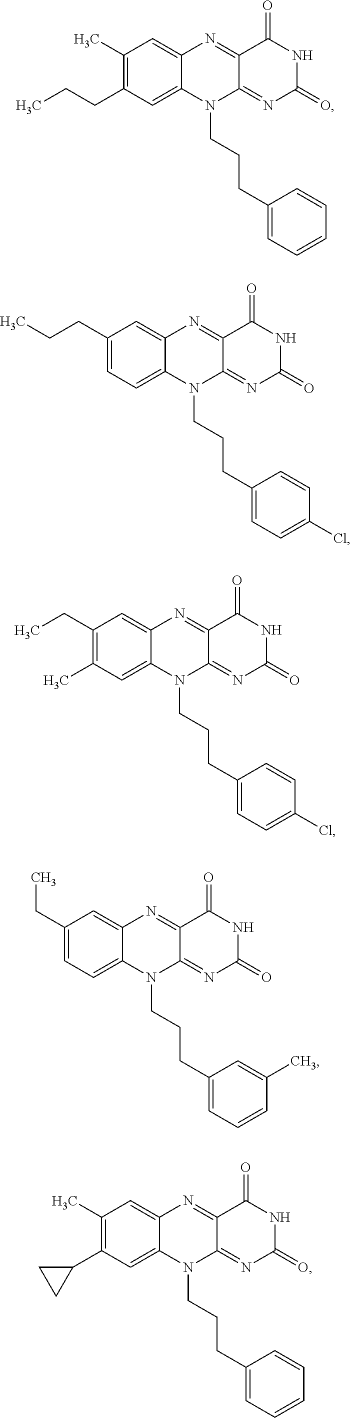

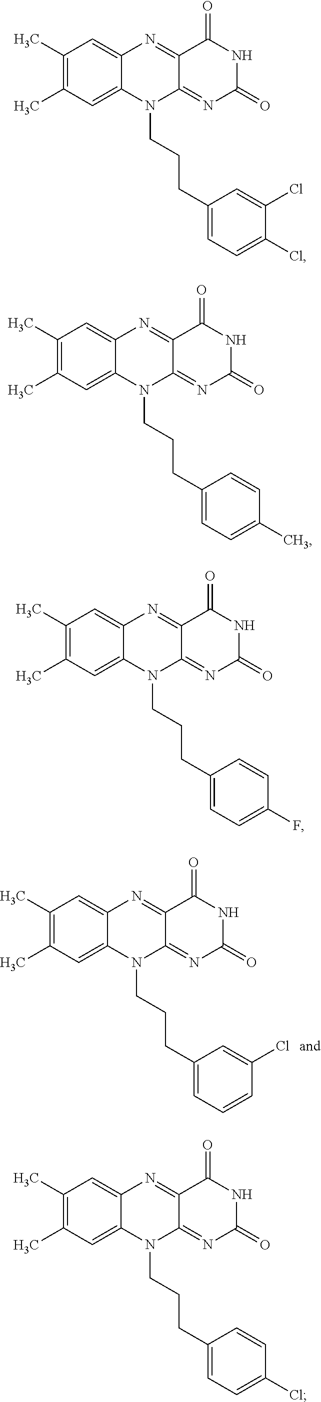

1.31. The compound according to any of the preceding formulae wherein said compound is selected from:

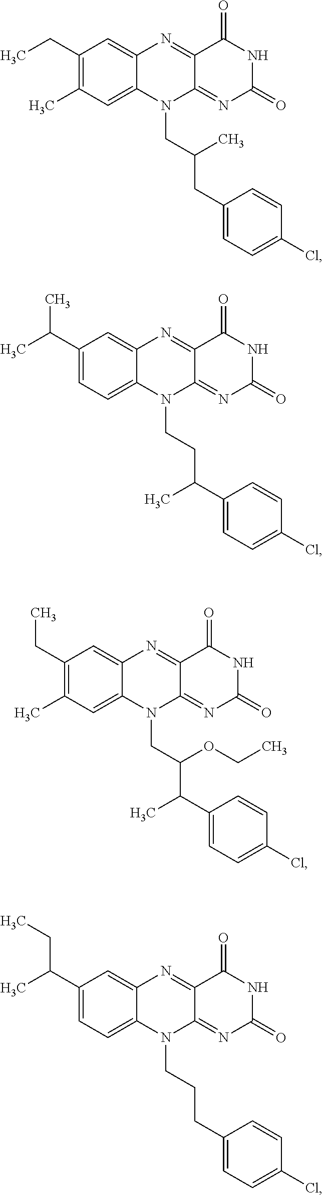 [SMILES: CCc1cc2nc3c(=O)[nH]c(=O)nc-3n(CC(C)Cc3ccc(Cl)cc3)c2cc1C ; CC(C)c1ccc2c(c1)nc1c(=O)[nH]c(=O)nc-1n2CCC(C)c1ccc(Cl)cc1 ; CCOC(Cn1c2nc(=O)[nH]c(=O)c-2nc2cc(CC)c(C)cc21)C(C)c1ccc(Cl)cc1 ; CCC(C)c1ccc2c(c1)nc1c(=O)[nH]c(=O)nc-1n2CCCc1ccc(Cl)cc1]

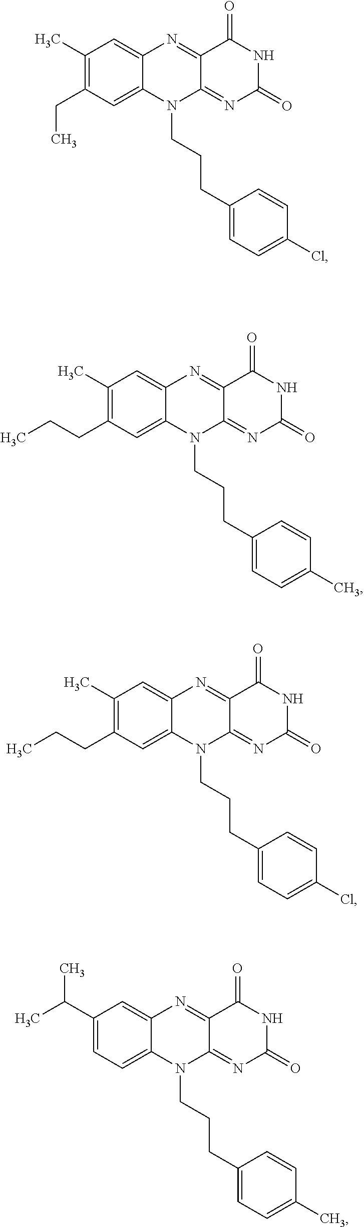 [SMILES: CCc1cc2c(cc1C)nc1c(=O)[nH]c(=O)nc-1n2CCCc1ccc(Cl)cc1 ; CCCc1cc2c(cc1C)nc1c(=O)[nH]c(=O)nc-1n2CCCc1ccc(C)cc1 ; CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1]

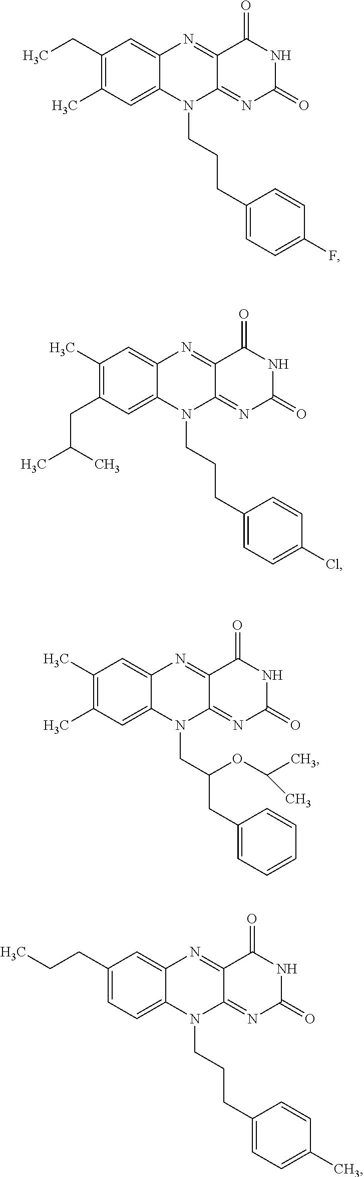

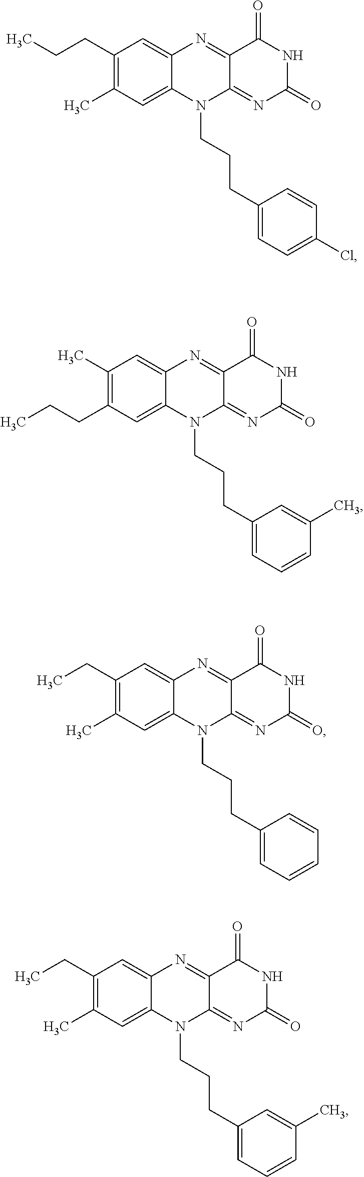 [SMILES: CCCc1cc2nc3c(=O)[nH]c(=O)nc-3n(CCCc3ccc(Cl)cc3)c2cc1C ; CCCc1cc2c(cc1C)nc1c(=O)[nH]c(=O)nc-1n2CCCc1cccc(C)c1 ; CCc1cc2nc3c(=O)[nH]c(=O)nc-3n(CCCc3ccccc3)c2cc1C ; CCc1cc2nc3c(=O)[nH]c(=O)nc-3n(CCCc3cccc(C)c3)c2cc1C]

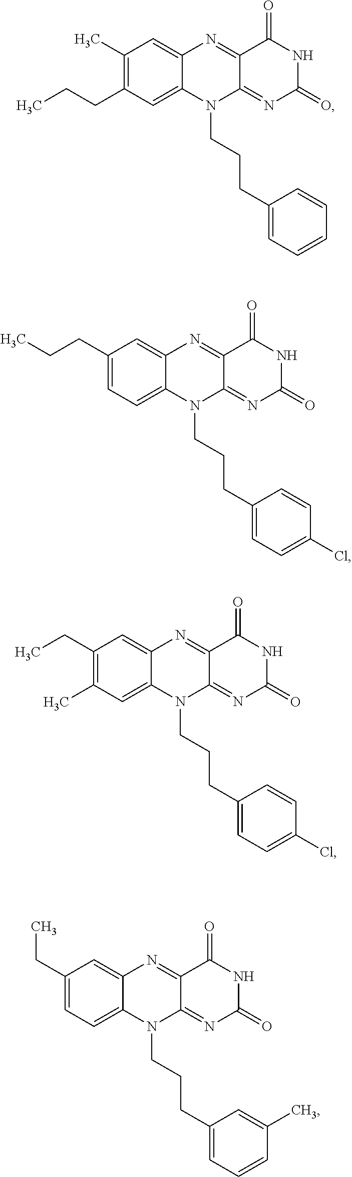 [SMILES: CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1 ; CCc1cc2nc3c(=O)[nH]c(=O)nc-3n(CCCc3ccc(Cl)cc3)c2cc1C ; CCc1ccc2c(c1)nc1c(=O)[nH]c(=O)nc-1n2CCCc1cccc(C)c1]

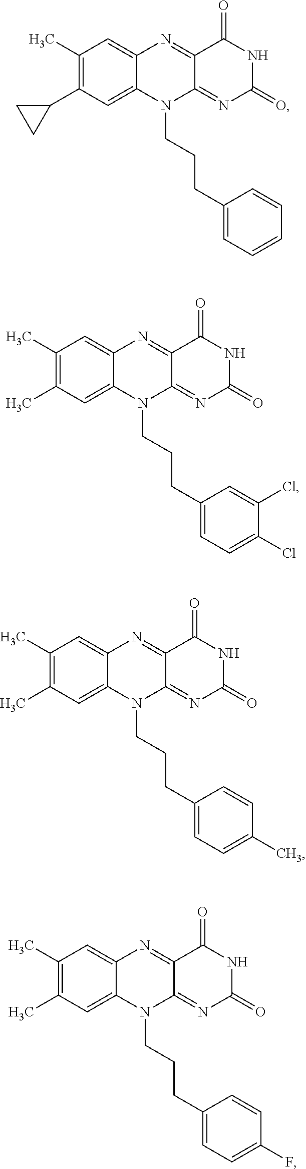 [SMILES: Cc1cc2nc3c(=O)[nH]c(=O)nc-3n(CCCc3ccccc3)c2cc1C1CC1 ; Cc1cc2nc3c(=O)[nH]c(=O)nc-3n(CCCc3ccc(Cl)c(Cl)c3)c2cc1C ; Cc1ccc(CCCn2c3nc(=O)[nH]c(=O)c-3nc3cc(C)c(C)cc32)cc1 ; Cc1cc2nc3c(=O)[nH]c(=O)nc-3n(CCCc3ccc(F)cc3)c2cc1C]

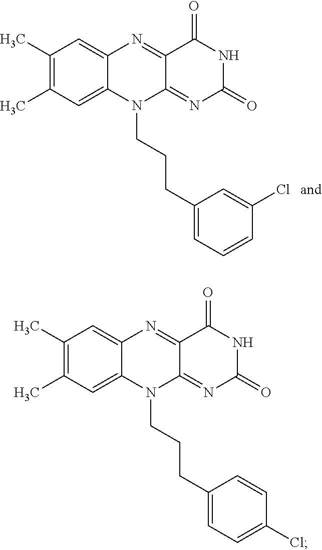

1.32. any of the preceding formulae wherein the compound of Formula Q binds to FMN and/or CD3299 riboswitch, e.g., with an Imax of greater than 20%, preferably greater than 30%, more preferably greater than 40%, still more preferably greater than 50% in an assay, for example, as described in Example A, and/or has a Minimum Inhibitory Concentration (MIC) of less than or equal to 64 μg/mL, more preferably less than or equal to 32 μg/mL, still more preferably, less than or equal to 16 μg/mL, for example, in an assay as described in Example B,

in free or salt form.

The invention further relates to a compound of Formula I as described in the following formulae:
1.33 a compound of formula I, wherein Alk is C₁₋₆alkylene (e.g., ethylene, n-propylene, n-butylene, n-pentylene) optionally substituted with one or more C₁₋₄alkyl, —N(R_{c})(R_{d}); or Alk is C₁₋₆alkylene (e.g., n-propylene, n-butylene, n-pentylene) optionally substituted with one hydroxy or C₁₋₄alkoxy group;
1.34 a compound of Formula 1 or 1.33, wherein Alk is C₂₋₅alkylene (e.g., ethylene, n-propylene, n-butylene, n-pentylene) optionally substituted as described in formula 1.33;
1.35 a compound of Formula I or any of 1.33-1.34, wherein Alk is C₃₋₄alkylene (e.g., n-propylene, n-butylene) optionally substituted as described in formula 1.33;
1.36 a compound of Formula I or any of 1.33-1.35, wherein Alk is selected from a group consisting of ethylene, n-propylene, n-butylene and n-pentylene, optionally substituted as described in formula 1.33;
1.37 a compound of Formula I or any of 1.33-1.36, wherein Alk is selected from a group consisting of ethylene, n-propylene, n-butylene, n-pentylene, —CH₂CH(OH)CH₂—, —CH₂CH₂CH(OH)—, —CH₂CH(NH₂)CH₂— and CH₂CH(N(CH₃)₂)CH₂—;
1.38 a compound of Formula I or any of 1.33-1.36, wherein Alk is ethylene, n-propylene or n-butylene;
1.39 a compound of Formula I or any of 1.33-1.36, wherein Alk is n-propylene or n-butylene;
1.40 a compound of Formula I or any of 1.33-1.39, wherein X is a single bond, —S— or —O—;
1.41 a compound of Formula I or any of 1.33-1.39, wherein X is a single bond;
1.42 a compound of Formula I or any of 1.33-1.39, wherein X is —S—;
1.43 a compound of Formula I or any of 1.33-1.39, wherein X is —O—;
1.44 a compound of Formula I or any of 1.33-1.43, wherein -Alk-X— is selected from a group consisting of ethylene, n-propylene, n-butylene, n-pentylene, CH₂CH(OH)CH₂—, —CH₂CH₂CH(OH)—, —CH₂CH(NH₂)CH₂—, CH₂CH(N(CH₃)₂)CH₂—, —CH₂CH₂O— and —CH₂CH₂S—;
1.45 a compound of Formula I or any of 1.33-1.44, wherein A is aryl (e.g., phenyl) or aryl-C₁₋₄alkyl (e.g., benzyl), wherein the aryl group of said aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
1.46 a compound of Formula I or any of 1.33-1.45, wherein A is aryl (e.g., phenyl) optionally substituted as disclosed in formula 1.45;
1.47 a compound of Formula I or any of 1.33-1.45, wherein A is phenyl optionally substituted as disclosed in formula 1.45;
1.48 a compound of Formula I or any of 1.33-1.47, wherein A is phenyl;
1.49 a compound of Formula I or any of 1.33-1.47, wherein A is phenyl substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
1.50 formula 1.49, wherein A is phenyl substituted with one or more substituent selected from a group consisting of methoxy, hydroxy, chloro, fluoro, methyl, CF₃, —OCF₃ and —OCH₂CH₂N(CH₃)(CH₃);
1.51 any of formulae 1.45-1.49, wherein A is phenyl, 4-methoxyphenyl, 4-hydroxyphenyl, 4-(2-dimethylaminoethoxy)-phenyl, 3-methoxyphenyl, 4-chlorophenyl, 3-chlorophenyl, 3,5-difluorophenyl, 3-hydroxyphenyl, 2-fluorophenyl, 4-fluorophenyl, 4-methylphenyl, 3-methylphenyl, 2-methylphenyl, 2,6-difluorophenyl, 3-trifluoromethylphenyl, 3,4-difluoromethyl, 3-trifluoromethoxyphenyl, 4-trifluoromethoxyphenyl, 4-methoxyphenyl, 3-chloro-4-fluorophenyl and 3,4-dichlorophenyl;
1.52 a compound of Formula I or any of 1.33-1.44, wherein A is aryl (e.g., phenyl) or aryl-C₁₋₄alkyl (e.g., benzyl), wherein the aryl group of said aryl or arylalkyl is substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
1.53 a compound of Formula I or any of 1.33-1.45, wherein
   -Alk is an n-propylene or n-butylene, optionally substituted with one or more C₁₋₄alkyl, —N(R_{c})(R_{d}) or optionally substituted with one hydroxy or C₁₋₄alkoxy group,
   —X— is a single bond, —O— or —S—, and
   A is phenyl optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
1.54 a compound of Formula I or any of 1.33-1.45, wherein
   -Alk is an n-propylene or n-butylene, optionally substituted with one or more C₁₋₄alkyl, —N(R_{c})(R_{d}) or optionally substituted with one hydroxy or C₁₋₄alkoxy group,
   —X— is a single bond, and
   A is phenyl optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
1.55 a compound of Formula I or any of 1.33-1.45, wherein
   -Alk is an n-propylene or n-butylene,
   —X— is a single bond, and
   A is phenyl optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
1.56 a compound of Formula I or any of 1.33-1.45, wherein
   -Alk is an n-propylene or n-butylene,
   —X— is a single bond, and
   A is phenyl optionally substituted with one or more C₁₋₄alkyl (e.g., methyl) or halo (e.g., Cl, F);
1.57 a compound of Formula I or any of 1.33-1.45, wherein
   -Alk is an n-propylene,
   —X— is a single bond, and
   A is phenyl optionally substituted with one or more C₁₋₄alkyl (e.g., methyl) or halo (e.g., Cl, F);
1.58 a compound of Formula I or any of 1.33-1.45, wherein A is aryl-C₁₋₄alkyl (e.g., benzyl) optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
1.59 a compound of Formula I or any of 1.33-1.58, wherein -Alk-X-A is selected from any of the following:

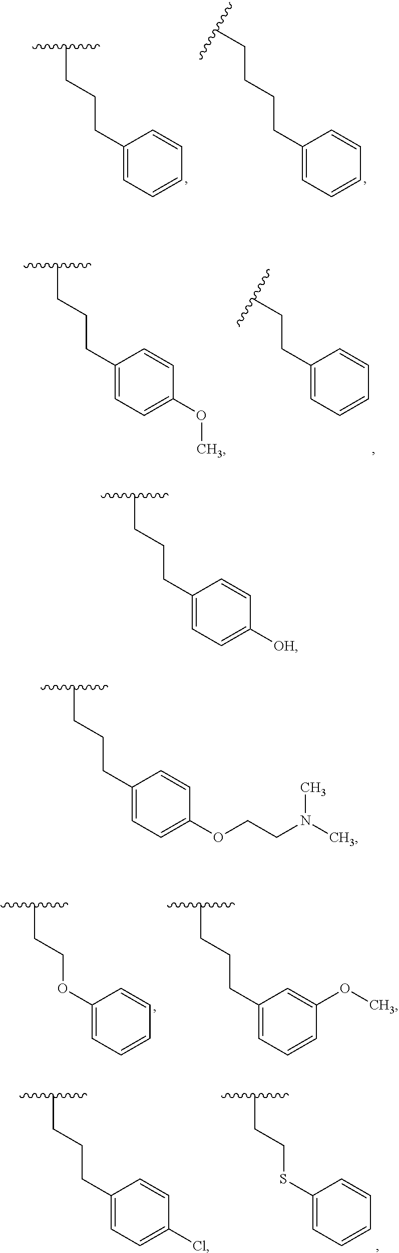

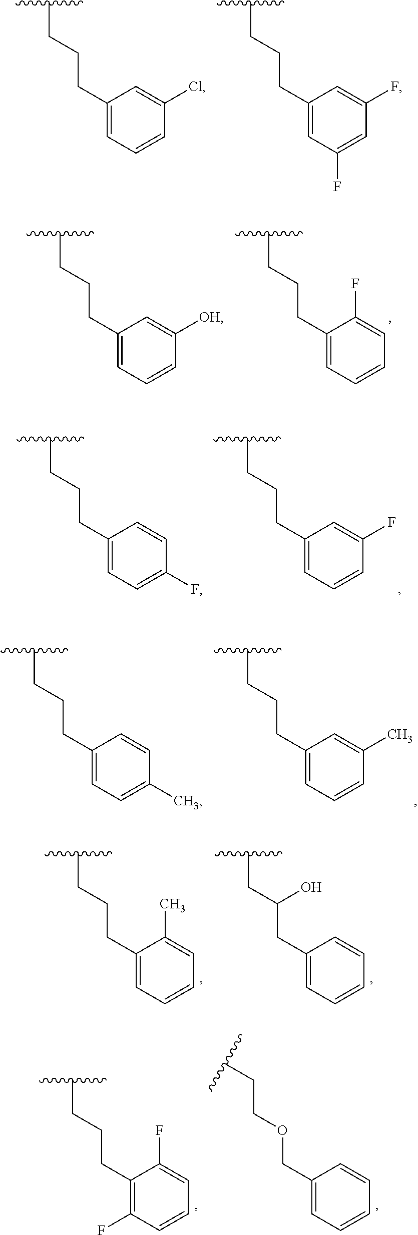

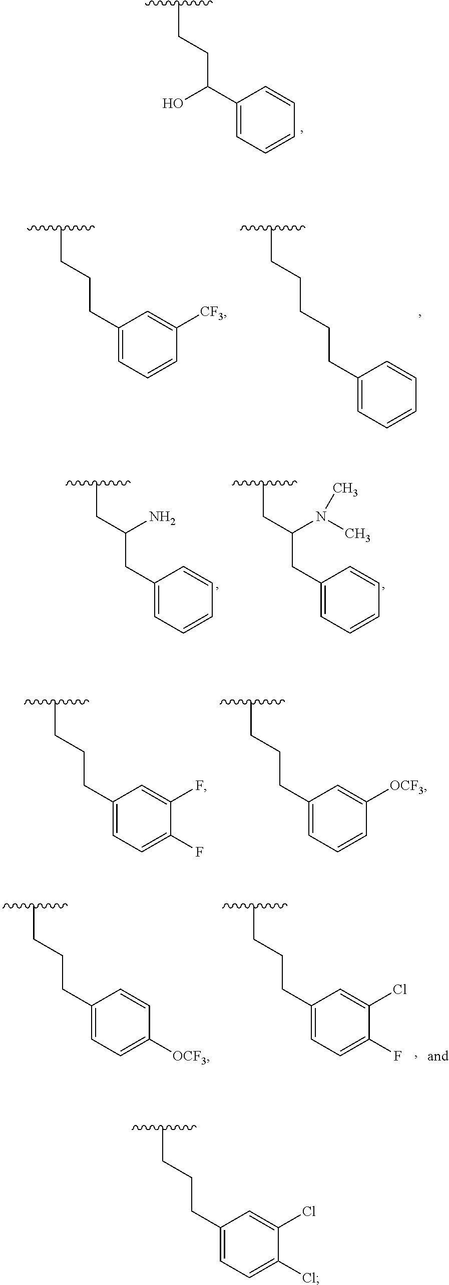

1.60 a compound of Formula I or any of formulae 1.33-1.59, wherein R₁ is H, C₁₋₄alkyl (e.g., methyl) or C₁₋₄alkoxy (e.g., methoxy);
1.61 a compound of Formula I or any of 1.33-1.60, wherein R₁ is H,
1.62 a compound of Formula I or any of 1.33-1.60, wherein R₁ is C₁₋₄alkyl (e.g., methyl);
1.63 a compound of Formula I or any of 1.33-1.60, wherein R₁ is methyl;
1.64 a compound of Formula I or any of 1.33-1.63, wherein R₂ is H, (e.g., methyl), —C₀₋₄alkyl-C₃₋₈cycloalkyl (e.g., cyclopropyl), —C₀₋₄alkyl-N(Rₐ)(R_{b}), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy;
1.65 a compound of Formula I or any of 1.33-1.63, wherein R₂ is H, C₁₋₄alkyl (e.g., methyl), —C₀₋₄alkyl-C₃₋₈cycloalkyl (e.g., cyclopropyl), N(Rₐ)(R_{b}), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy;
1.66 a compound of Formula I or any of 1.33-1.63, wherein R₂ is H, C₁₋₄alkyl (e.g., methyl), —C₀₋₄alkyl-C₃₋₈cycloalkyl (e.g., cyclopropyl), —C₁₋₄alkyl-N(Rₐ)(R_{b}), C₁₋₄alkoxy (e.g., methoxy), C₃₋₈heterocycloalkyl (e.g., pyrrolidinyl, for example pyrrolidin-1-yl) wherein said heterocycloalkyl is optionally substituted with one or more hydroxy;
1.67 a compound of Formula I or any of 1.33-1.64, wherein R₂ is selected from a group consisting of H, C₁₋₄alkyl, (e.g., methyl), —C₀₋₄alkyl-C₃₋₈cycloalkyl (e.g., cyclopropyl) and halo (e.g., Cl);
1.68 a compound of Formula I or any of 1.33-1.64, wherein R₂ is H,
1.69 a compound of Formula I or any of 1.33-1.64, wherein R₂ is C₁₋₄alkyl (e.g., methyl);
1.70 a compound of Formula I or any of 1.33-1.64, wherein R₂ is methyl;
1.71 a compound of Formula I or any of 1.33-1.64, wherein R₂ is —C₀₋₄alkyl-C₃₋₈ cycloalkyl (e.g., cyclopropyl);
1.72 a compound of Formula I or any of 1.33-1.64, wherein R₂ is halo (e.g., Cl);
1.73 a compound of Formula 1 or any of 1.33-1.64, wherein R₁ and R₂ are selected from H, C₁₋₄alkyl (e.g., methyl) and —C₀₋₄alkyl-C₃₋₈cycloalkyl (e.g., cyclopropyl);
1.74 a compound of Formula I or any of 1.33-1.64, wherein R₁ and R₂ are both methyl;
1.75 a compound of Formula 1 or any of 1.33-1.64, wherein R₁ is H and R₂ is —C₀₋₄ alkyl-C₃₋₈cycloalkyl (e.g., cyclopropyl);
1.76 a compound of Formula I or any of 1.33-1.64, wherein R₁ and R₂ are linked so that together with the carbon atoms to which they are attached, they form a cyclic structure;
1.77 a compound of Formula I or any of 1.33-1.64, wherein R₁ and R₂ are methoxy and R₁ and R₂ are linked together so that together with the carbon atoms to which they are attached they form a cyclic structure (e.g., R₁ and R₂ are linked together to form ethylenedioxy);
1.78 a compound of Formula I or any of 1.33-1.77, wherein Rₐ and R_{b} are independently H, C₁₋₄alkyl (e.g., methyl), C₃₋₈cycloalkyl (e.g., cyclopropyl, cyclopentyl), C₁₋₄alkoxy-C₁₋₄alkyl (e.g., methoxyethyl), hydroxy-C₁₋₄alkyl (e.g., hydroxyethyl), N(R_{c})(R_{d})—C₁₋₄alkyl (e.g., dimethylaminoethyl);
1.79 a compound of Formula I or any of 1.33-1.78, wherein R_{c} and R_{d} are independently H or C₁₋₄alkyl (e.g., methyl);
1.80 a compound of Formula I or any of 1.33-1.79, wherein R_{c} and R_{d} are H,
1.81 a compound of Formula I or any of 1.33-1.79, wherein R_{c} and R_{d} are C₁₋₄alkyl (e.g., methyl);
1.82 a compound of Formula I or any of 1.33-1.79, wherein R_{c} is H and R_{d} is C₁₋₄alkyl (e.g., methyl);
1.83 a compound of Formula I or any of formulae 1.33-1.82, wherein
   Alk is C₁₋₆alkylene (e.g., ethylene, n-propylene, n-butylene, n-pentylene) optionally substituted with one or more C₁₋₄alkyl, —N(R_{c})(R_{d}); or
   Alk is C₁₋₆alkylene (e.g., n-propylene, n-butylene, n-pentylene) optionally substituted with one hydroxy or C₁₋₄alkoxy group;
   X is a single bond, —S— or —O—;
   A is aryl (e.g., phenyl) or aryl-C₁₋₄alkyl (e.g., benzyl), wherein the aryl group of aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H, C₁₋₄alkyl (e.g., methyl) or C₁₋₄alkoxy (e.g., methoxy);
   R₂ is H, C₁₋₄alkyl (e.g., methyl), —C₀₋₄alkyl-C₃₋₈cycloalkyl (e.g., cyclopropyl), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy; or
   Optionally, R₁ and R₂ are linked together to form a cyclic structure (e.g., R₁ and R₂ are linked together to from ethylenedioxy);
   R_{c} and R_{d} are independently H or C₁₋₄alkyl (e.g., methyl);
1.84 a compound of Formula I or any of formulae 1.33-1.82, wherein
   Alk is C₂₋₅alkylene (e.g., ethylene, n-propylene, n-butylene, n-pentylene) optionally substituted with one or more C₁₋₄alkyl, —N(R_{c})(R_{d}); or
   Alk is C₂₋₅alkylene (e.g., n-propylene, n-butylene, n-pentylene) optionally substituted with one hydroxy or C₁₋₄alkoxy group;
   X is a single bond, —S— or —O—;
   A is aryl (e.g., phenyl) or aryl-C₁₋₄alkyl (e.g., benzyl), wherein the aryl group of aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H, C₁₋₄alkyl (e.g., methyl) or C₁₋₄alkoxy (e.g., methoxy);
   R₂ is H, C₁₋₄alkyl (e.g., methyl), —C₀₋₄alkyl-C₃₋₈cycloalkyl (e.g., cyclopropyl), —C₁₋₄alkyl-N(Rₐ)(R_{b}), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy; or
   Optionally, R₁ and R₂ are linked together to form a cyclic structure (e.g., R₁ and R₂ are linked together to from ethylenedioxy);
   Rₐ and R_{b} are independently H, C₁₋₄alkyl (e.g., methyl), C₃₋₈cycloalkyl (e.g., cyclopropyl, cyclopentyl), C₁₋₄alkoxy-C₁₋₄alkyl (e.g., methoxyethyl), hydroxy-C₁₋₄alkyl (e.g., hydroxyethyl), N(R_{c})(R_{d})—C₁₋₄alkyl (e.g., dimethylaminoethyl);
   R_{c} and R_{d} are independently H or C₁₋₄alkyl (e.g., methyl);
1.85 a compound of Formula I or any of formulae 1.33-1.82, wherein R₂ is:
   H, C₁₋₄alkyl (e.g., methyl), —C₀₋₄alkyl-C₃₋₈cycloalkyl (e.g., cyclopropyl), —C₁alkyl-N(Rₐ)(R_{b}), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy;
1.86 a compound of Formula I or any of formulae 1.33-1.82, wherein
   Alk is C₃₋₄alkylene (e.g., n-propylene, n-butylene) optionally substituted with one or more C₁₋₄alkyl, —N(R_{c})(R_{d}); or
   Alk is C₃₋₄alkylene (e.g., n-propylene, n-butylene) optionally substituted with one hydroxy or C₁₋₄alkoxy group;
   X is a single bond, —S— or —O—;
   A is aryl (e.g., phenyl) optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H, C₁₋₄alkyl (e.g., methyl) or C₁₋₄alkoxy (e.g., methoxy);
   R₂ is H, C₁₋₄alkyl (e.g., methyl), —C₀₋₄alkyl-C₃₋₈cycloalkyl (e.g., cyclopropyl) or halo (e.g., Cl);
   R_{c} and R_{d} are independently H or C₁₋₄alkyl (e.g., methyl);
1.87 a compound of Formula I or any of formulae 1.33-1.82, wherein
   Alk is C₃₋₄alkylene (e.g., n-propylene, n-butylene) optionally substituted with one or more C₁₋₄alkyl, —N(R_{c})(R_{d}); or
   Alk is C₃₋₄alkylene (e.g., n-propylene, n-butylene) optionally substituted with one hydroxy or C₁₋₄alkoxy group;
   X is a single bond, —S— or —O—;
   A is phenyl optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H, C₁₋₄alkyl (e.g., methyl) or C₁₋₄alkoxy (e.g., methoxy);
   R₂ is H, C₁₋₄alkyl (e.g., methyl), —C₀₋₄alkyl-C₃₋₈cycloalkyl (e.g., cyclopropyl) or halo (e.g., Cl);
   R_{c} and R_{d} are independently H or C₁₋₄alkyl (e.g., methyl);
1.88 a compound of Formula I or any of formulae 1.33-1.82, wherein
   Alk is C₃₋₄alkylene (e.g., n-propylene, n-butylene) optionally substituted with one or more C₁₋₄alkyl, —N(R_{c})(R_{d}); or
   Alk is C₃₋₄alkylene (e.g., n-propylene, n-butylene) optionally substituted with one hydroxy or C₁₋₄alkoxy group;
   X is a single bond;
   A is aryl (e.g., phenyl) optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H or C₁₋₄alkyl (e.g., methyl);
   R₂ is H, C₁₋₄alkyl (e.g., methyl), —C₀₋₄alkyl-C₃₋₈cycloalkyl (e.g., cyclopropyl) or halo (e.g., Cl);
   R_{c} and R_{d} are independently H or C₁₋₄alkyl (e.g., methyl);
1.89 a compound of Formula I or any of formulae 1.33-1.82, wherein
   Alk is C₃₋₄alkylene (e.g., n-propylene, n-butylene);
   X is a single bond;
   A is aryl (e.g., phenyl) optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
   R₁ is H or C₁₋₄alkyl (e.g., methyl);
   R₂ is H, C₁₋₄alkyl (e.g., methyl), —C₀₋₄alkyl-C₃₋₈cycloalkyl (e.g., cyclopropyl) or halo (e.g., Cl);
   R_{c} and R_{d} are independently H or C₁₋₄alkyl (e.g., methyl);
1.90 a compound of Formula I or any of formulae 1.33-1.82, wherein
   Alk is C₃₋₄alkylene (e.g., n-propylene, n-butylene);
   X is a single bond;
   A is aryl (e.g., phenyl);
   R₁ is H or C₁₋₄alkyl (e.g., methyl);
   R₂ is H, C₁₋₄alkyl (e.g., methyl), —C₀₋₄alkyl-C₃₋₈cycloalkyl (e.g., cyclopropyl) or halo (e.g., Cl);
1.91 a compound of Formula I or any of formulae 1.33-1.82, wherein
   Alk is C₃₋₄alkylene (e.g., n-propylene, n-butylene);
   X is a single bond;
   A is aryl (e.g., phenyl);
   R₁ is C₁₋₄alkyl (e.g., methyl);
   R₂ is C₁₋₄alkyl (e.g., methyl) or —C₀₋₄alkyl-C₃₋₈cycloalkyl (e.g., cyclopropyl);
1.92 a compound of Formula I or any of formulae 1.33-1.82, wherein
   Alk is C₃₋₄alkylene (e.g., n-propylene, n-butylene);
   X is —S—;
   A is aryl (e.g., phenyl) optionally substituted with one or more C₁₋₄alkyl (e.g., methyl) or halo (e.g., Cl, F);
   R₁ is C₁₋₄alkyl (e.g., methyl);
   R₂ is C₁₋₄alkyl (e.g., methyl) or —C₀₋₄alkyl-C₃₋₈cycloalkyl (e.g., cyclopropyl);
1.93 a compound of Formula I or any of formulae 1.33-1.82, wherein
   Alk is C₃₋₄alkylene (e.g., n-propylene, n-butylene);
   X is a single bond;
   A is aryl (e.g., phenyl) optionally substituted with one or more C₁₋₄alkyl (e.g., methyl) or halo (e.g., Cl, F);
   R₁ is H or C₁₋₄alkyl (e.g., methyl);
   R₂ is —C₀₋₄alkyl-C₃₋₈cycloalkyl (e.g., cyclopropyl);
1.94 a compound of Formula I or any of formulae 1.33-1.82, wherein
   Alk is C₃₋₄alkylene (e.g., n-propylene, n-butylene);
   X is a single bond;
   A is aryl (e.g., phenyl) substituted with one or more C₁₋₄alkyl (e.g., methyl) or halo (e.g., Cl, F);
   R₁ is H or C₁₋₄alkyl (e.g., methyl);
   R₂ is H, C₁₋₄alkyl (e.g., methyl), —C₀₋₄alkyl-C₃₋₈cycloalkyl (e.g., cyclopropyl);
1.95 a compound of Formula I or any of formulae 1.33-1.82, wherein
   Alk is C₃₋₄alkylene (e.g., n-propylene, n-butylene);
   X is a single bond;
   A is aryl (e.g., phenyl) substituted with one or more methyl, Cl or F;
   R₁ is H or C₁₋₄alkyl (e.g., methyl);
   R₂ is H, C₁₋₄alkyl (e.g., methyl) or —C₀₋₄alkyl-C₃₋₈cycloalkyl (e.g., cyclopropyl);
1.96 a compound of Formula I or any of formulae 1.33-1.82, wherein
   Alk is C₃₋₄alkylene (e.g., n-propylene, n-butylene);
   X is a single bond;
   A is 4-chlorophenyl, 3-chloromethyl or 4-methylphenyl;
   R₁ is H or C₁₋₄alkyl (e.g., methyl);
   R₂ is H, C₁₋₄alkyl (e.g., methyl) or —C₀₋₄alkyl-C₃₋₈cycloalkyl (e.g., cyclopropyl);
1.97 any of formulae 1.33-1.96, wherein the compound of Formula I is selected from any of the following:

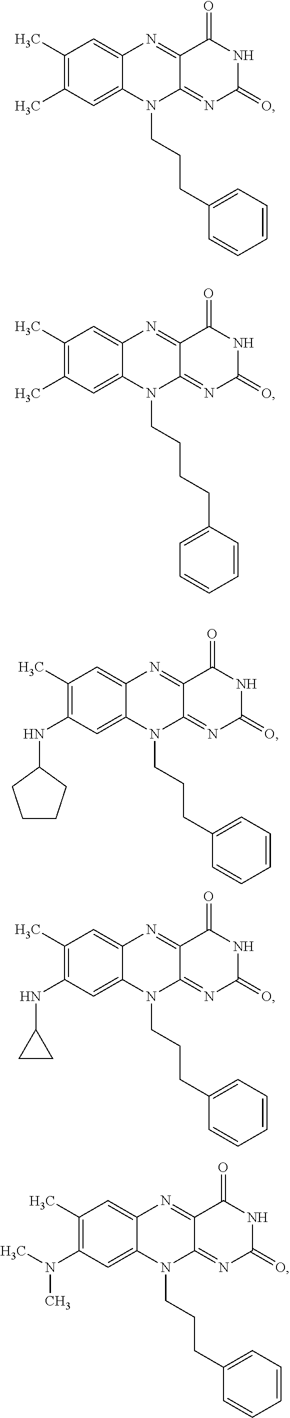

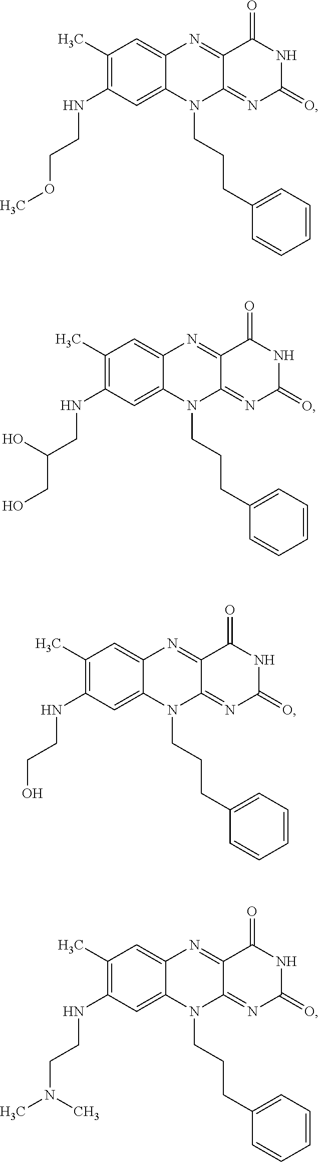 [SMILES: COCCNc1cc2c(cc1C)nc1c(=O)[nH]c(=O)nc-1n2CCCc1ccccc1 ; Cc1cc2nc3c(=O)[nH]c(=O)nc-3n(CCCc3ccccc3)c2cc1NCC(O)CO ; Cc1cc2nc3c(=O)[nH]c(=O)nc-3n(CCCc3ccccc3)c2cc1NCCO ; Cc1cc2nc3c(=O)[nH]c(=O)nc-3n(CCCc3ccccc3)c2cc1NCCN(C)C]

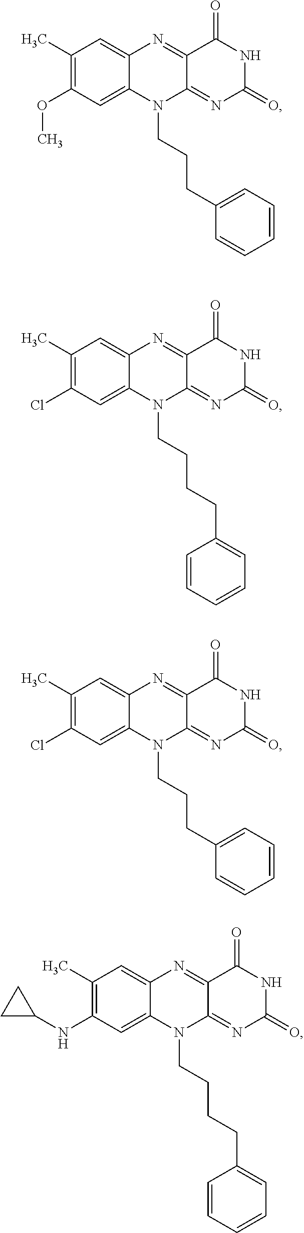

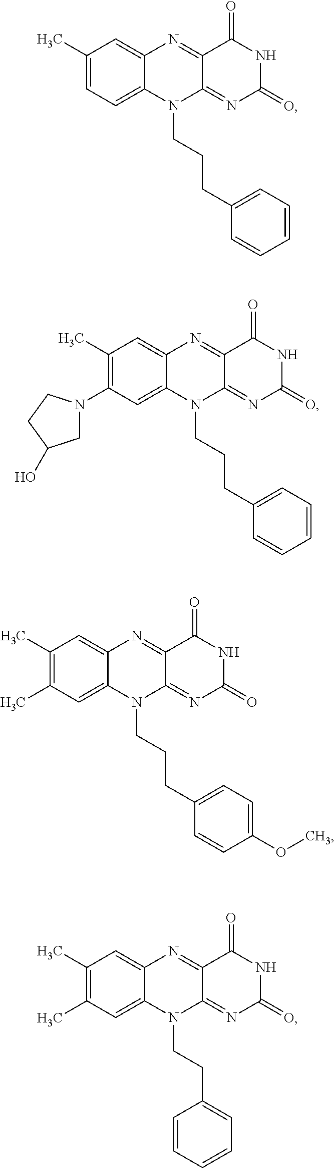 [SMILES: Cc1ccc2c(c1)nc1c(=O)[nH]c(=O)nc-1n2CCCc1ccccc1 ; Cc1cc2nc3c(=O)[nH]c(=O)nc-3n(CCCc3ccccc3)c2cc1N1CCC(O)C1 ; COc1ccc(CCCn2c3nc(=O)[nH]c(=O)c-3nc3cc(C)c(C)cc32)cc1 ; Cc1cc2nc3c(=O)[nH]c(=O)nc-3n(CCc3ccccc3)c2cc1C]

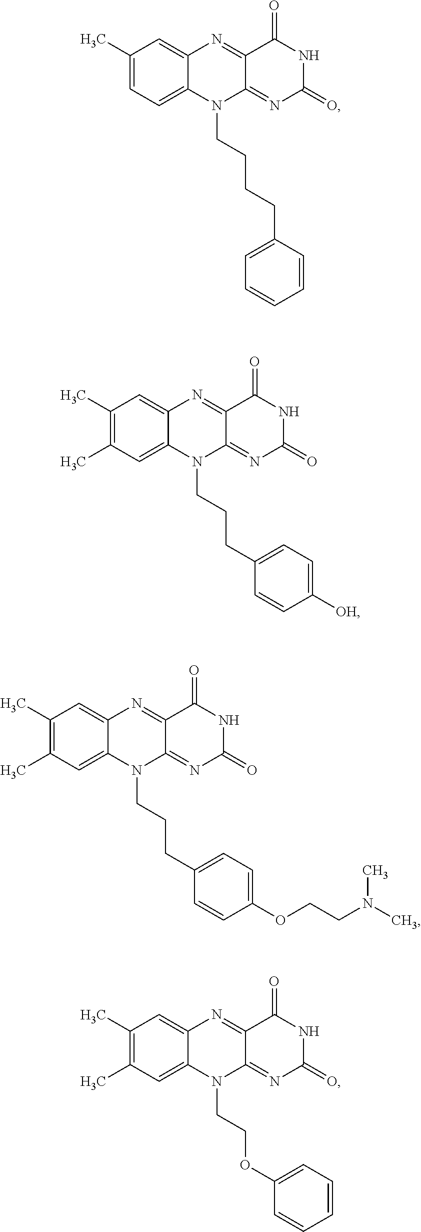 [SMILES: Cc1ccc2c(c1)nc1c(=O)[nH]c(=O)nc-1n2CCCCc1ccccc1 ; Cc1cc2nc3c(=O)[nH]c(=O)nc-3n(CCCc3ccc(O)cc3)c2cc1C ; Cc1cc2nc3c(=O)[nH]c(=O)nc-3n(CCCc3ccc(OCCN(C)C)cc3)c2cc1C ; Cc1cc2nc3c(=O)[nH]c(=O)nc-3n(CCOc3ccccc3)c2cc1C]

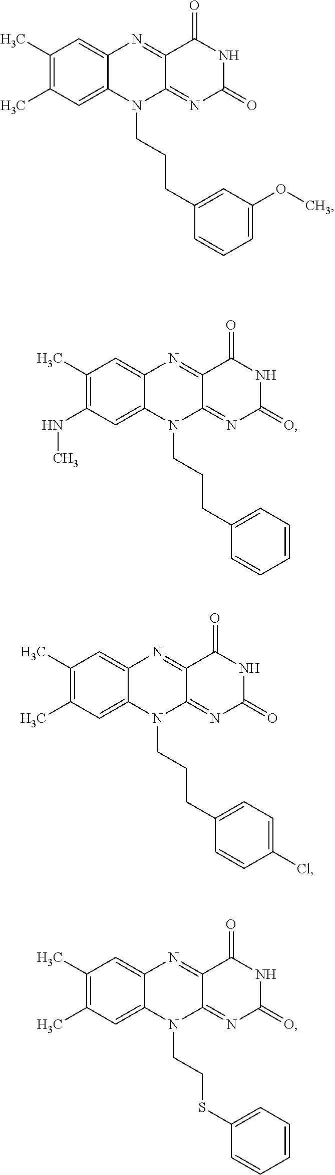 [SMILES: COc1cccc(CCCn2c3nc(=O)[nH]c(=O)c-3nc3cc(C)c(C)cc32)c1 ; CNc1cc2c(cc1C)nc1c(=O)[nH]c(=O)nc-1n2CCCc1ccccc1 ; Cc1cc2nc3c(=O)[nH]c(=O)nc-3n(CCCc3ccc(Cl)cc3)c2cc1C ; Cc1cc2nc3c(=O)[nH]c(=O)nc-3n(CCSc3ccccc3)c2cc1C]

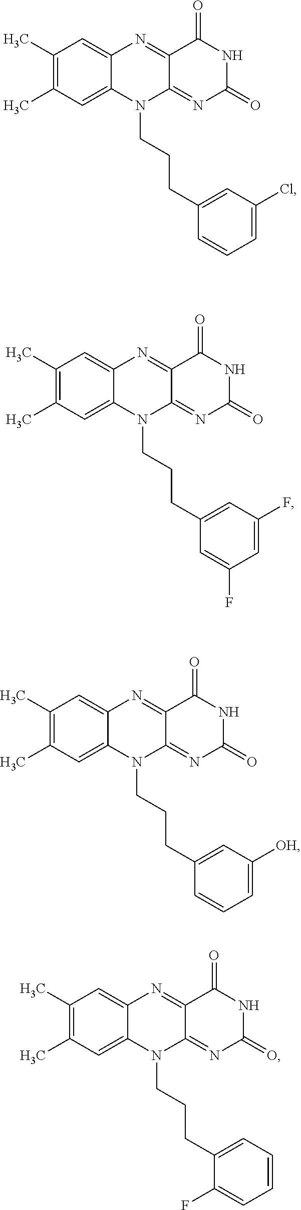

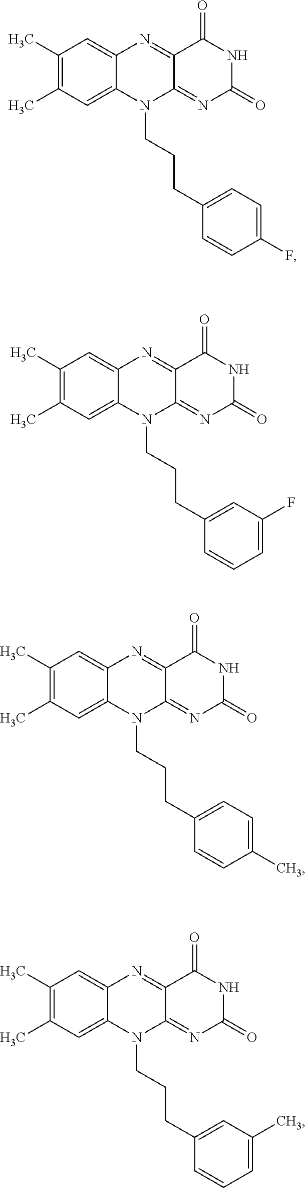 [SMILES: Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(F)c3)c2cc1C ; Cc1ccc(CCCn2c3nc(=O)[nH]c(=O)c-3nc3cc(C)c(C)cc32)cc1 ; Cc1cccc(CCCn2c3nc(=O)[nH]c(=O)c-3nc3cc(C)c(C)cc32)c1]

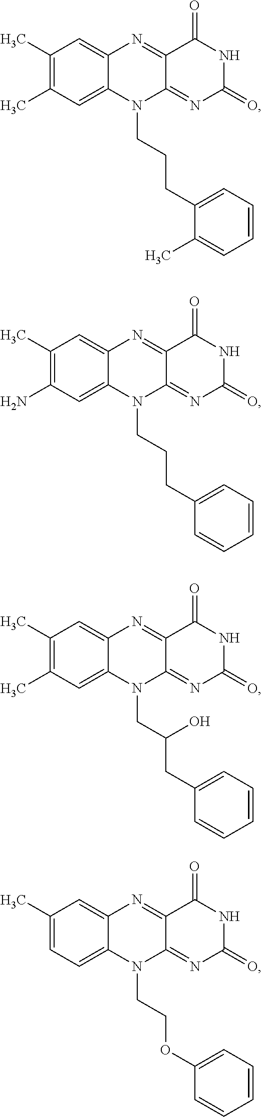

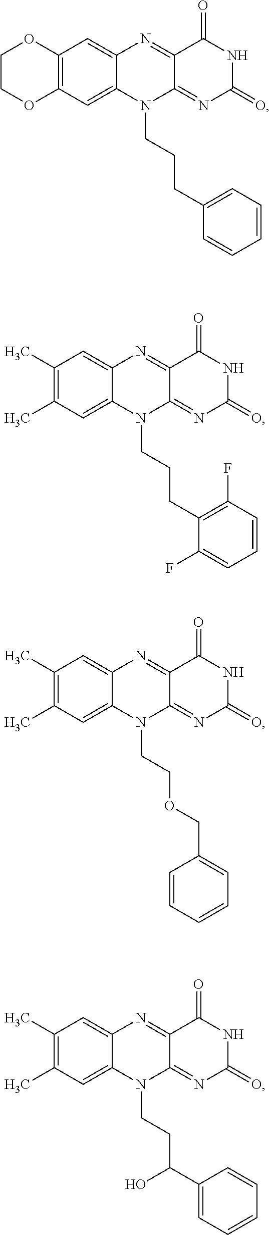 [SMILES: O=c1nc2n(CCCc3ccccc3)c3cc4c(cc3nc-2c(=O)[nH]1)OCCO4 ; Cc1cc2nc3c(=O)[nH]c(=O)nc-3n(CCCc3c(F)cccc3F)c2cc1C ; Cc1cc2nc3c(=O)[nH]c(=O)nc-3n(CCOCc3ccccc3)c2cc1C ; Cc1cc2nc3c(=O)[nH]c(=O)nc-3n(CCC(O)c3ccccc3)c2cc1C]

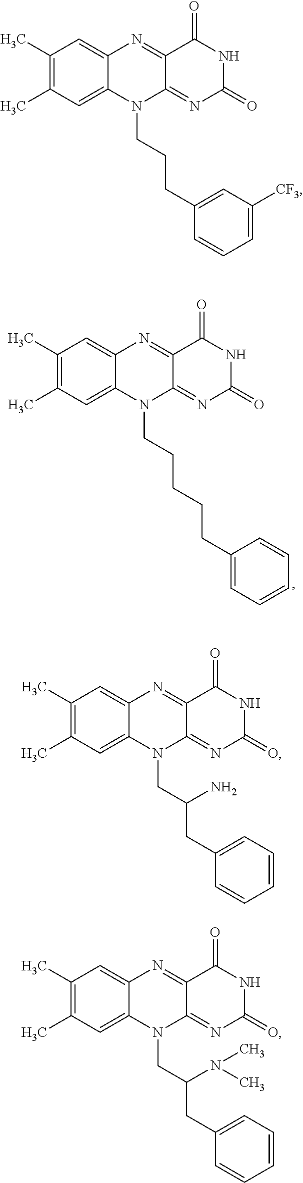

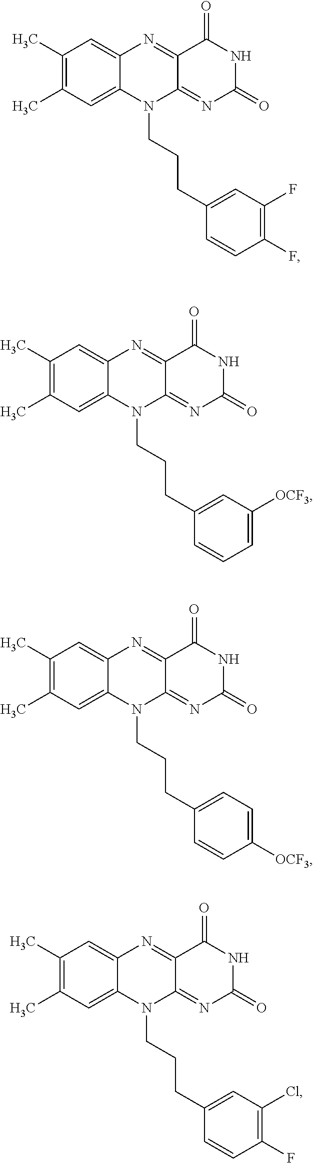 [SMILES: Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C ; Cc1cc2nc3c(=O)[nH]c(=O)nc-3n(CCCc3ccc(F)c(Cl)c3)c2cc1C]

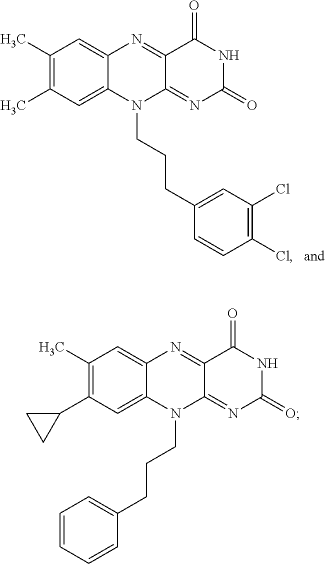 [SMILES: Cc1cc2nc3c(=O)[nH]c(=O)nc-3n(CCCc3ccc(Cl)c(Cl)c3)c2cc1C ; "and" ; Cc1cc2nc3c(=O)[nH]c(=O)nc-3n(CCCc3ccccc3)c2cc1C1CC1]

1.98 any of formulae 1.33-1.96, wherein the compound of Formula I is selected from any of the following:

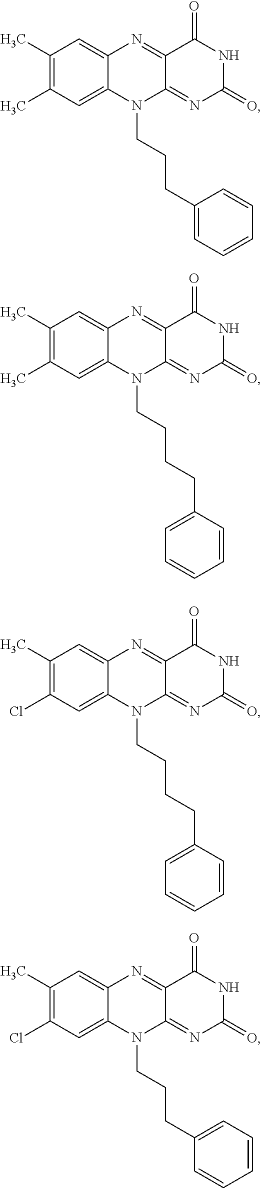

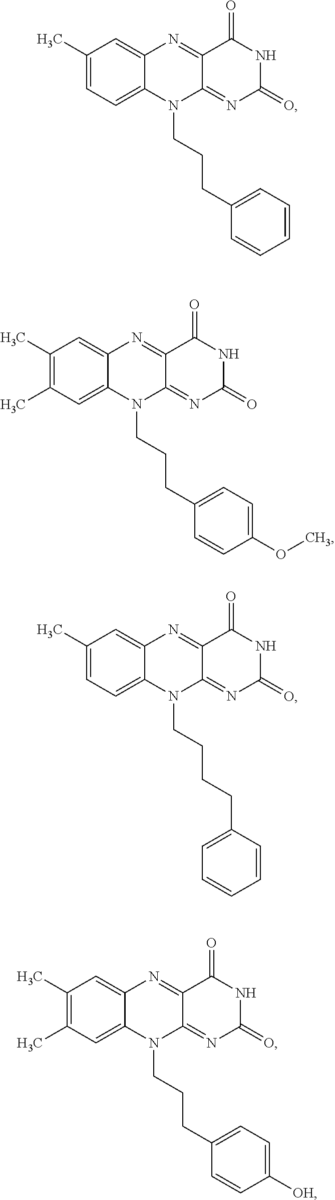

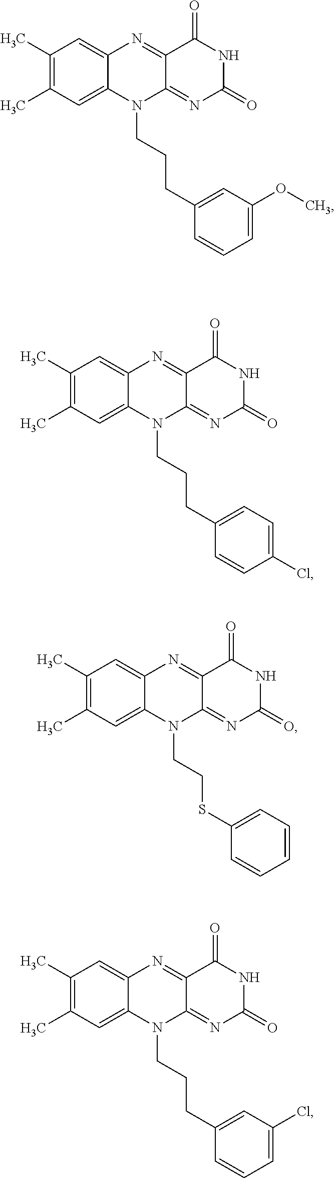 [SMILES: COc1cccc(CCCn2c3nc(=O)[nH]c(=O)c-3nc3cc(C)c(C)cc32)c1 ; Cc1cc2nc3c(=O)[nH]c(=O)nc-3n(CCCc3ccc(Cl)cc3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3cccc(Cl)c3)c2cc1C]

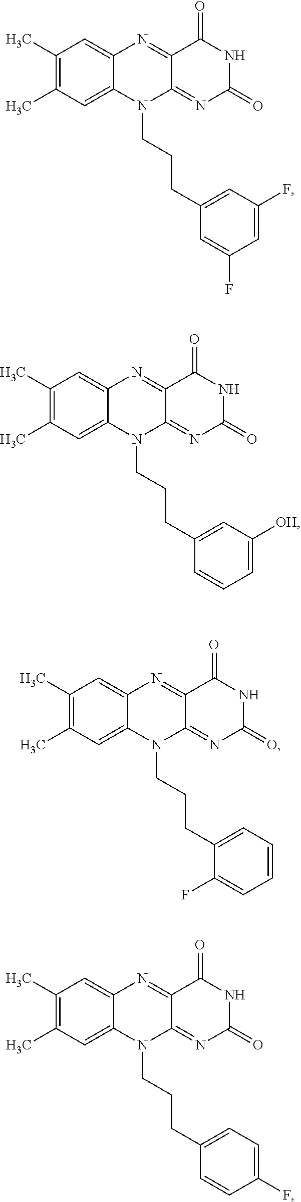 [SMILES: Cc1cc2nc3c(=O)[nH]c(=O)nc-3n(CCCc3cc(F)cc(F)c3)c2cc1C ; Cc1cc2nc3c(=O)[nH]c(=O)nc-3n(CCCc3cccc(O)c3)c2cc1C ; Cc1cc2nc3c(=O)[nH]c(=O)nc-3n(CCCc3ccccc3F)c2cc1C ; Cc1cc2nc3c(=O)[nH]c(=O)nc-3n(CCCc3ccc(F)cc3)c2cc1C]

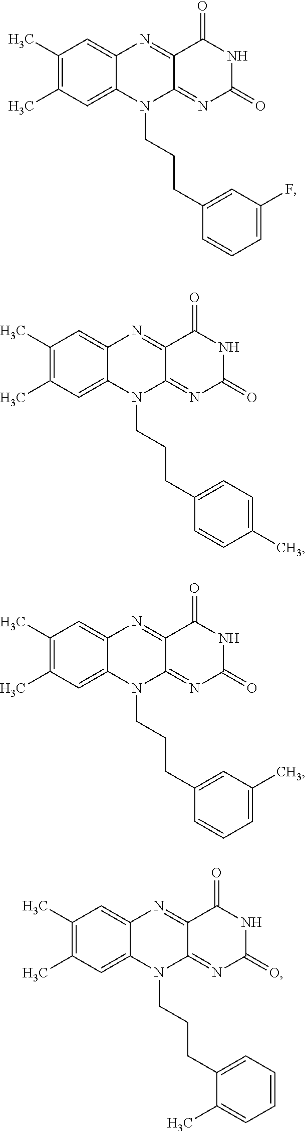

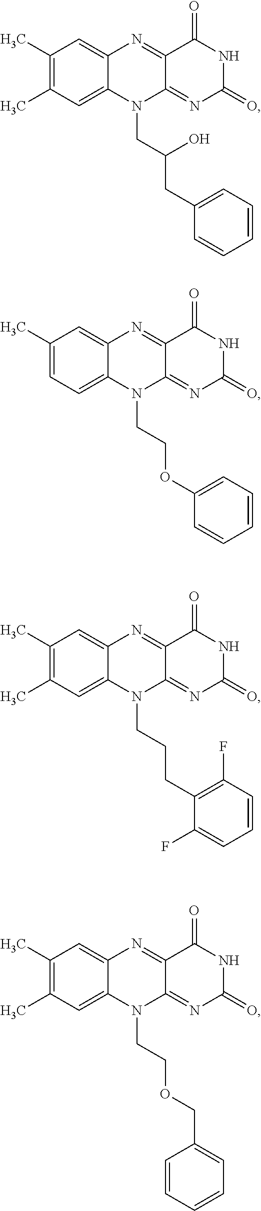 [SMILES: Cc1cc2nc3c(=O)[nH]c(=O)nc-3n(CC(O)Cc3ccccc3)c2cc1C ; Cc1ccc2c(c1)nc1c(=O)[nH]c(=O)nc-1n2CCOc1ccccc1 ; Cc1cc2nc3c(=O)[nH]c(=O)nc-3n(CCCc3c(F)cccc3F)c2cc1C ; Cc1cc2nc3c(=O)[nH]c(=O)nc-3n(CCOCc3ccccc3)c2cc1C]

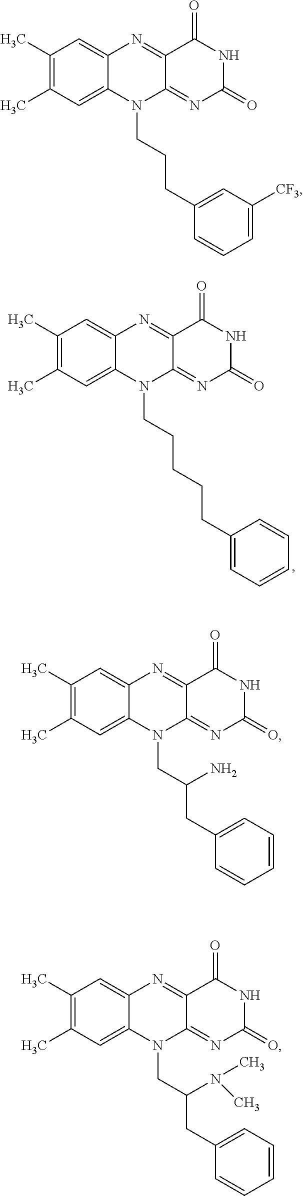

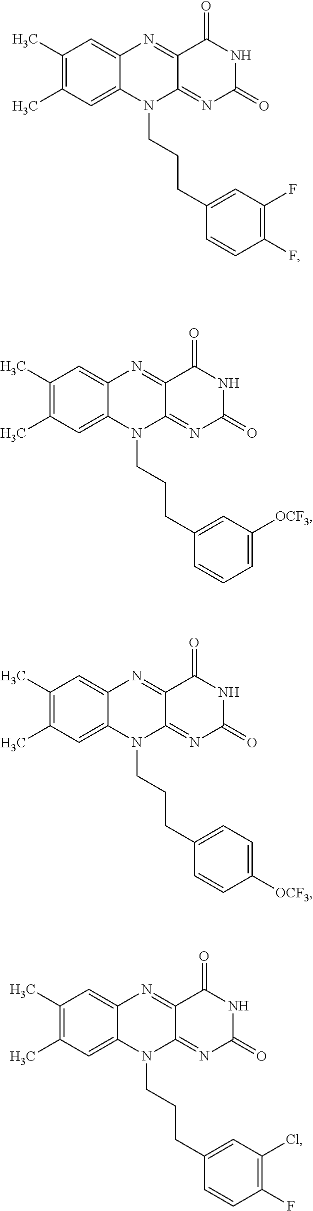 [SMILES: Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C ; Cc1cc2nc3c(=O)[nH]c(=O)nc-3n(CCCc3ccc(F)c(Cl)c3)c2cc1C]

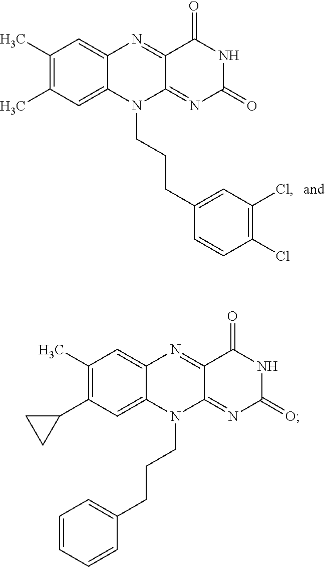 [SMILES: Cc1cc2nc3c(=O)[nH]c(=O)nc-3n(CCCc3ccc(Cl)c(Cl)c3)c2cc1C ; "and" ; Cc1cc2nc3c(=O)[nH]c(=O)nc-3n(CCCc3ccccc3)c2cc1C1CC1]

1.99 any of formulae 1.33-1.96, wherein the compound of Formula I is selected from any of the following:

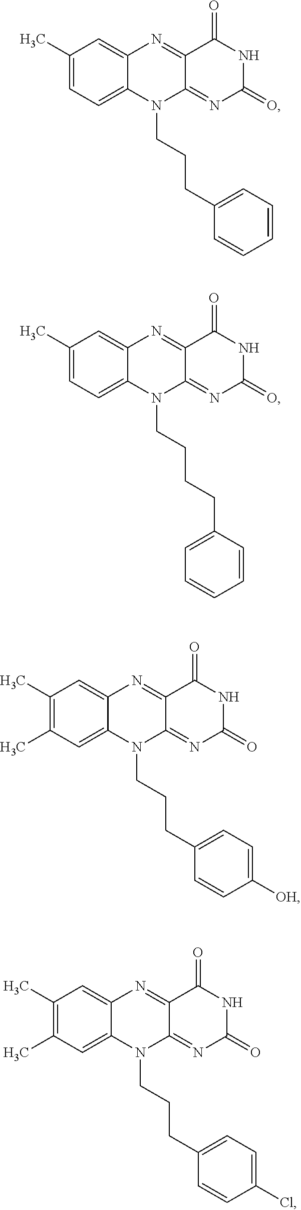 [SMILES: Cc1ccc2c(c1)nc1c(=O)[nH]c(=O)nc-1n2CCCc1ccccc1 ; Cc1ccc2c(c1)nc1c(=O)[nH]c(=O)nc-1n2CCCCc1ccccc1 ; Cc1cc2nc3c(=O)[nH]c(=O)nc-3n(CCCc3ccc(O)cc3)c2cc1C ; Cc1cc2nc3c(=O)[nH]c(=O)nc-3n(CCCc3ccc(Cl)cc3)c2cc1C]

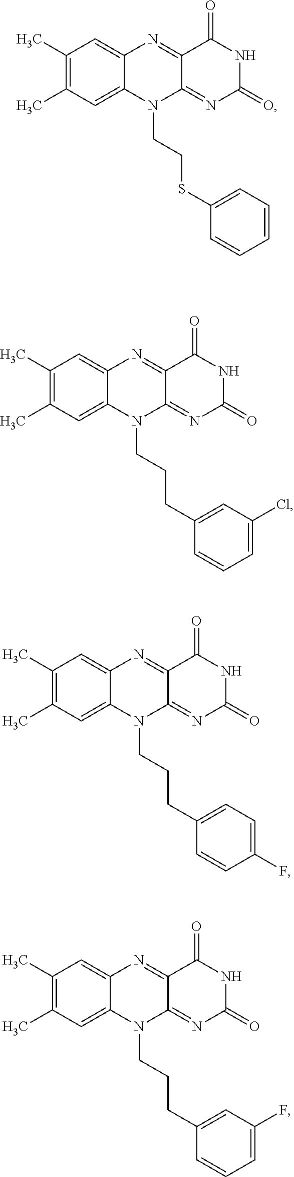 [SMILES: Cc1cc2nc3c(=O)[nH]c(=O)nc-3n(CCSc3ccccc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(F)c3)c2cc1C]

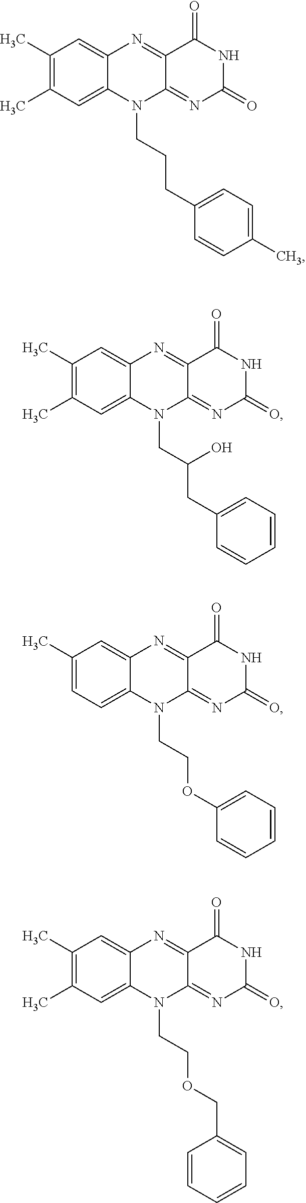 [SMILES: Cc1ccc(CCCn2c3nc(=O)[nH]c(=O)c-3nc3cc(C)c(C)cc32)cc1 ; Cc1cc2nc3c(=O)[nH]c(=O)nc-3n(CC(O)Cc3ccccc3)c2cc1C ; Cc1ccc2c(c1)nc1c(=O)[nH]c(=O)nc-1n2CCOc1ccccc1 ; Cc1cc2nc3c(=O)[nH]c(=O)nc-3n(CCOCc3ccccc3)c2cc1C]

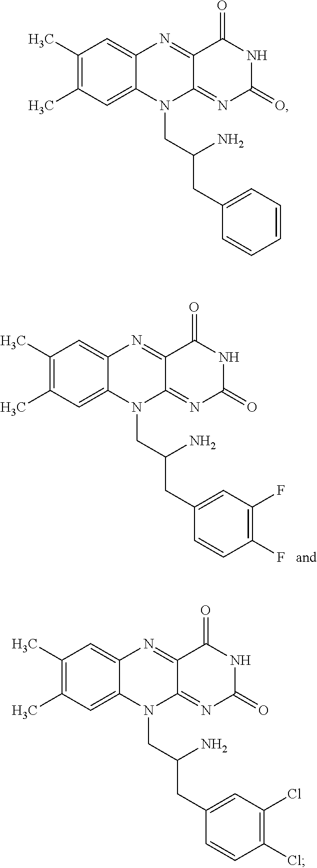 [SMILES: Cc1cc2nc3c(=O)[nH]c(=O)nc-3n(CC(N)Cc3ccccc3)c2cc1C ; Cc1cc2nc3c(=O)[nH]c(=O)nc-3n(CC(N)Cc3ccc(F)c(F)c3)c2cc1C ; "and" ; Cc1cc2nc3c(=O)[nH]c(=O)nc-3n(CC(N)Cc3ccc(Cl)c(Cl)c3)c2cc1C]

1.100 any of formulae 1.33-1.96, selected from any of the following:

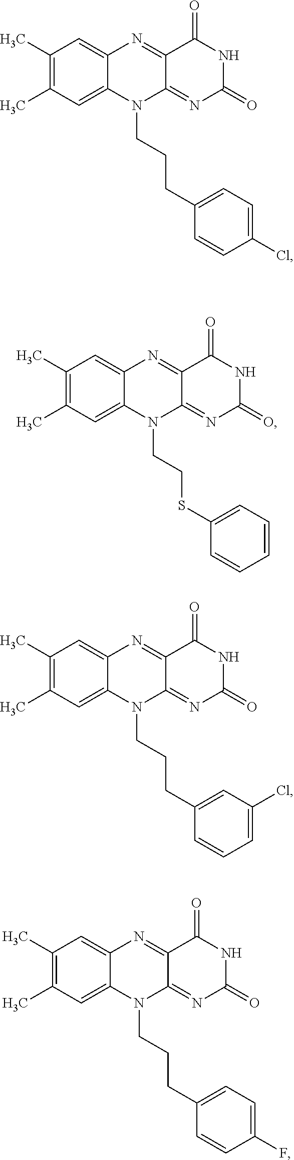 [SMILES: Cc1cc2nc3c(=O)[nH]c(=O)nc-3n(CCCc3ccc(Cl)cc3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCCc3ccc(F)cc3)c2cc1C]

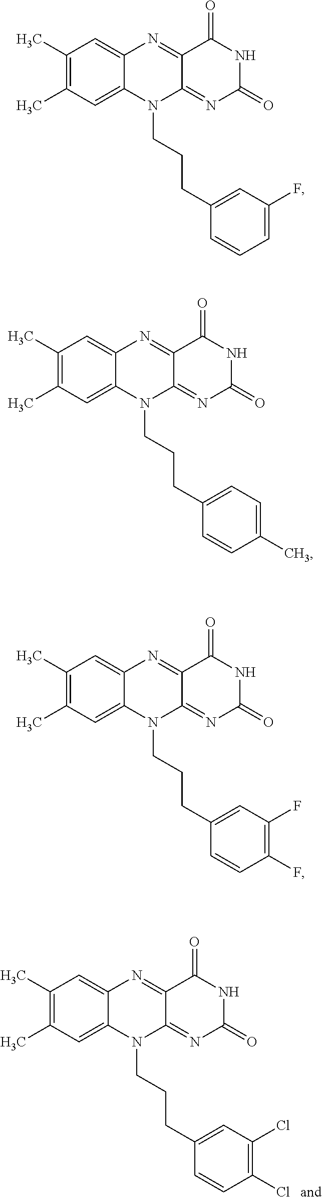 [SMILES: Cc1cc2nc3c(=O)[nH]c(=O)nc-3n(CCCc3cccc(F)c3)c2cc1C ; Cc1ccc(CCCn2c3nc(=O)[nH]c(=O)c-3nc3cc(C)c(C)cc32)cc1 ; Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3ccc(Cl)c(Cl)c3)c2cc1C ; "and"]

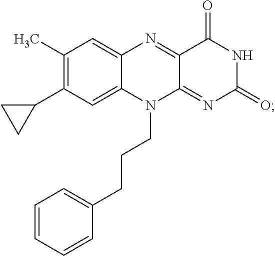 [SMILES: Cc1cc2nc3c(=O)[nH]c(=O)nc-3n(CCCc3ccccc3)c2cc1C1CC1]

1.101 any of formulae 1.33-1.96, selected from any of the following:

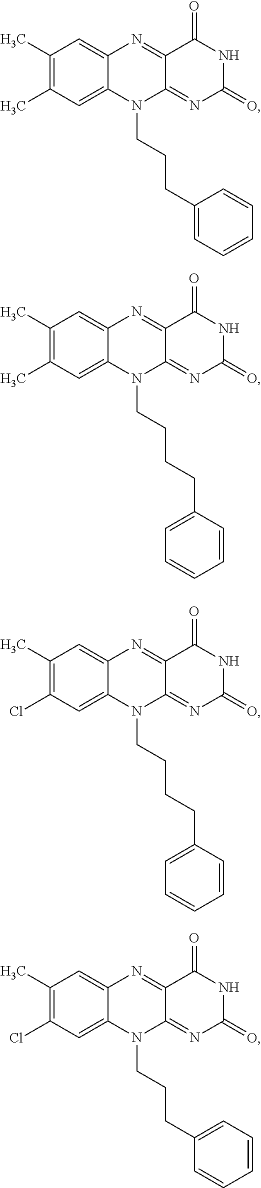

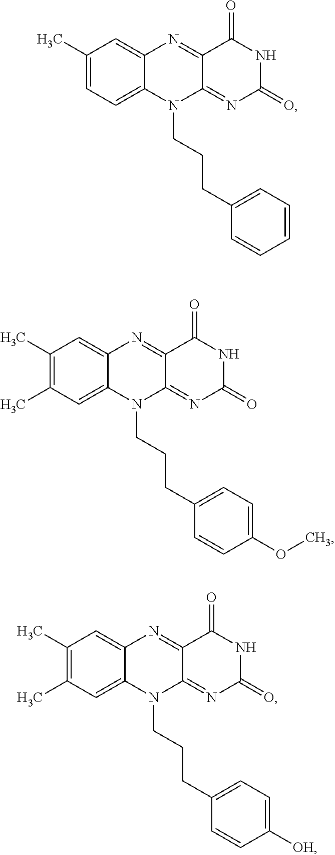

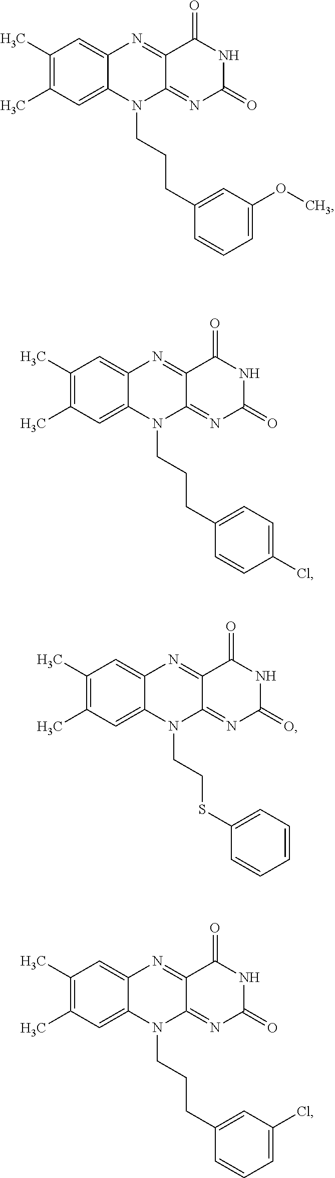 [SMILES: COc1cccc(CCCn2c3nc(=O)[nH]c(=O)c-3nc3cc(C)c(C)cc32)c1 ; Cc1cc2nc3c(=O)[nH]c(=O)nc-3n(CCCc3ccc(Cl)cc3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3cccc(Cl)c3)c2cc1C]

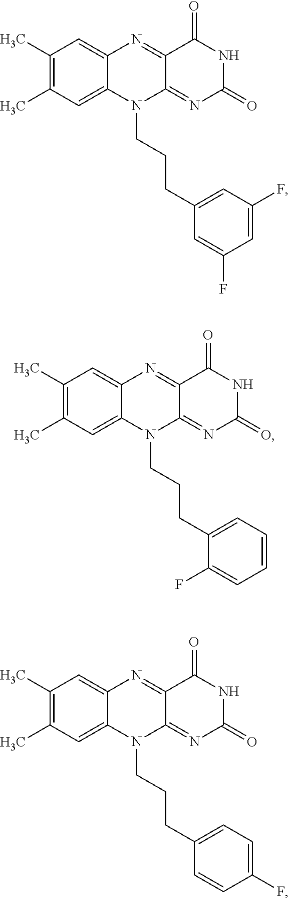 [SMILES: Cc1cc2nc3c(=O)[nH]c(=O)nc-3n(CCCc3cc(F)cc(F)c3)c2cc1C ; Cc1cc2nc3c(=O)[nH]c(=O)nc-3n(CCCc3ccccc3F)c2cc1C ; Cc1cc2nc3c(=O)[nH]c(=O)nc-3n(CCCc3ccc(F)cc3)c2cc1C]

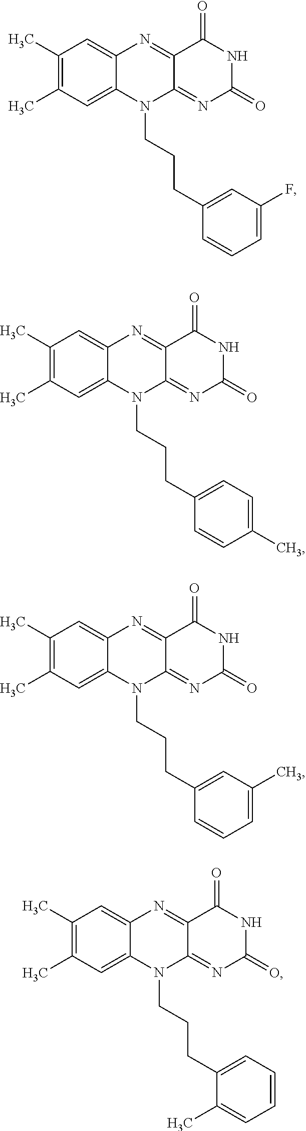

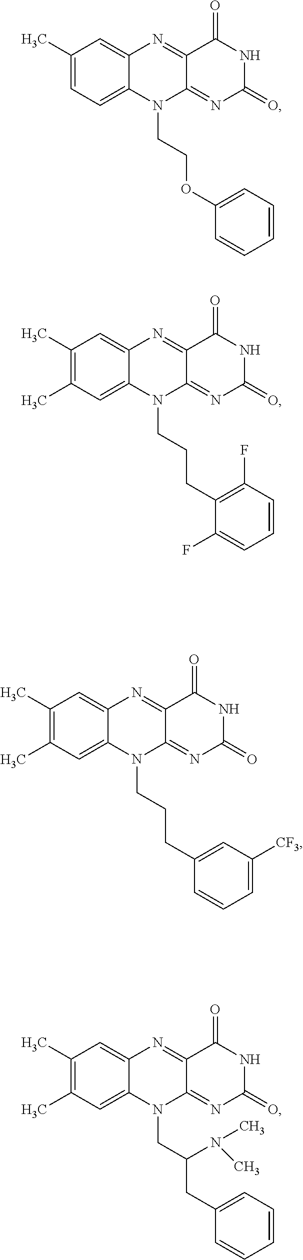 [SMILES: Cc1ccc2c(c1)nc1c(=O)[nH]c(=O)nc-1n2CCOc1ccccc1 ; Cc1cc2nc3c(=O)[nH]c(=O)nc-3n(CCCc3c(F)cccc3F)c2cc1C ; Cc1cc2nc3c(=O)[nH]c(=O)nc-3n(CCCc3cccc(C(F)(F)F)c3)c2cc1C ; Cc1cc2nc3c(=O)[nH]c(=O)nc-3n(CC(Cc3ccccc3)N(C)C)c2cc1C]

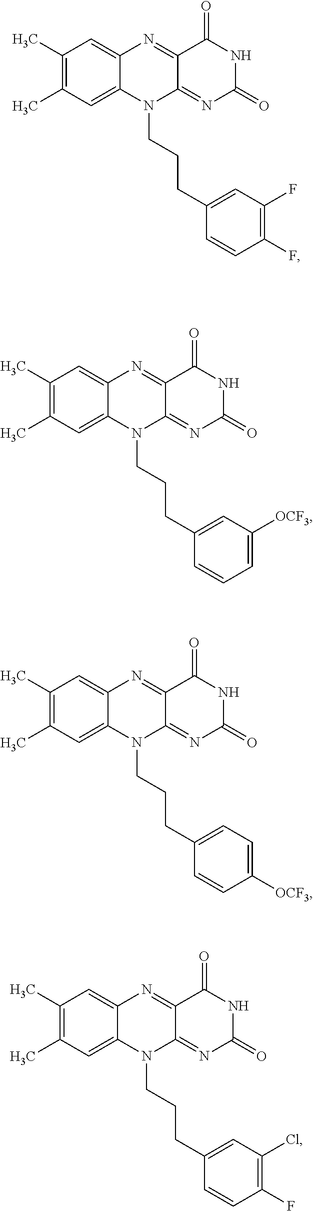 [SMILES: Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C ; Cc1cc2nc3c(=O)[nH]c(=O)nc-3n(CCCc3ccc(F)c(Cl)c3)c2cc1C]

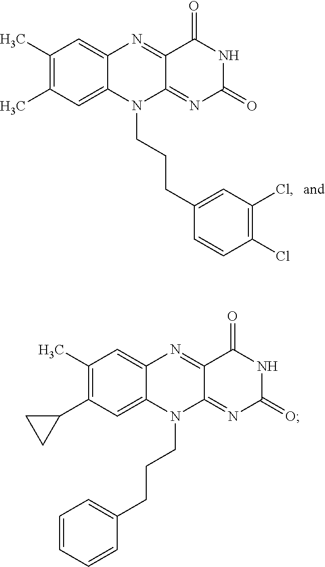 [SMILES: Cc1cc2nc3c(=O)[nH]c(=O)nc-3n(CCCc3ccc(Cl)c(Cl)c3)c2cc1C ; "and" ; Cc1cc2nc3c(=O)[nH]c(=O)nc-3n(CCCc3ccccc3)c2cc1C1CC1]

1.102 formula 1.101, wherein the compound of Formula I is selected from any of the following:

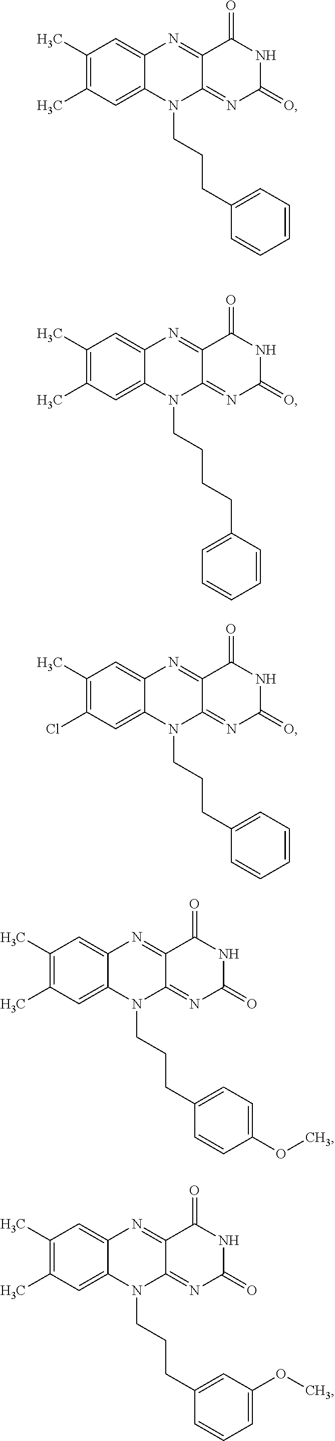

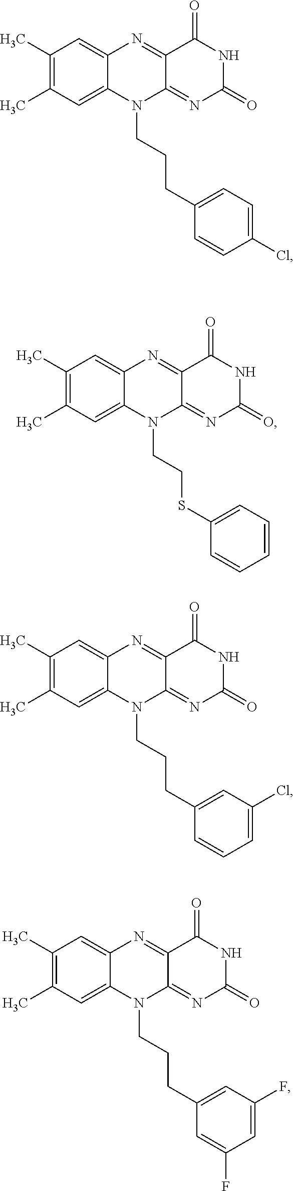 [SMILES: Cc1cc2nc3c(=O)[nH]c(=O)nc-3n(CCCc3ccc(Cl)cc3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCCc3cc(F)cc(F)c3)c2cc1C]

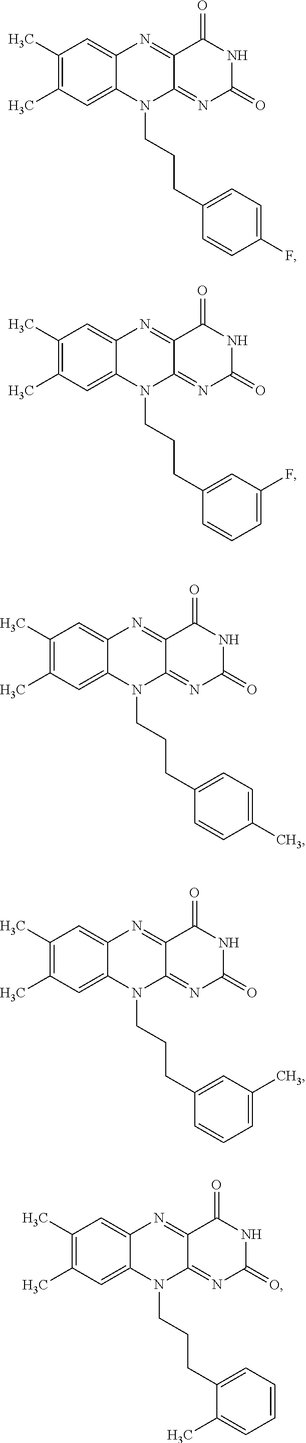

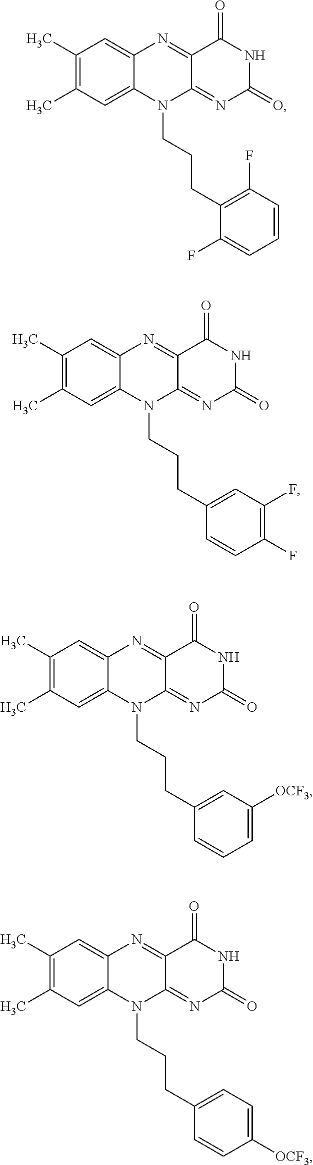 [SMILES: Cc1cc2nc3c(=O)[nH]c(=O)nc-3n(CCCc3c(F)cccc3F)c2cc1C ; Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C]

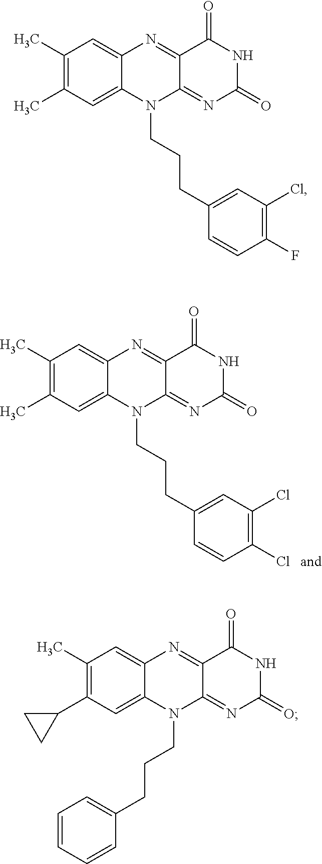 [SMILES: Cc1cc2nc3c(=O)[nH]c(=O)nc-3n(CCCc3ccc(F)c(Cl)c3)c2cc1C ; Cc1cc2nc3c(=O)[nH]c(=O)nc-3n(CCCc3ccc(Cl)c(Cl)c3)c2cc1C ; "and" ; Cc1cc2nc3c(=O)[nH]c(=O)nc-3n(CCCc3ccccc3)c2cc1C1CC1]

1.103 a compound of Formula I or any of formulae 1.33-1.98, wherein the compound of Formula I is selected from any of the following:

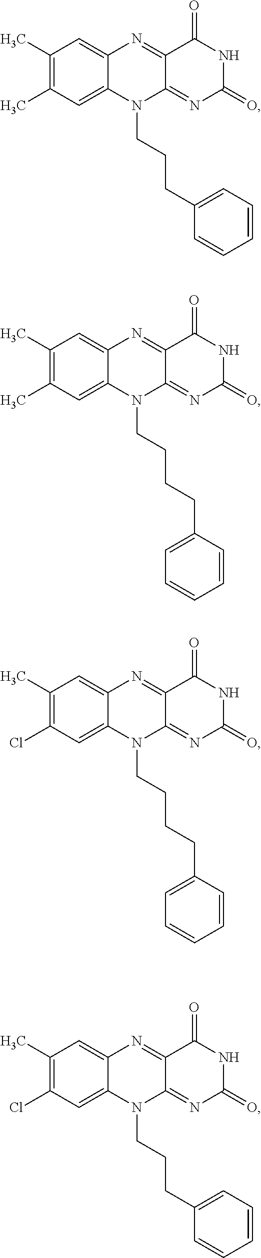

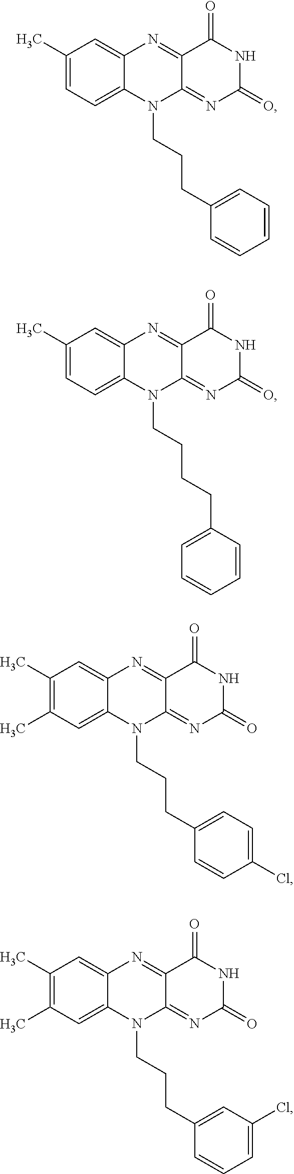

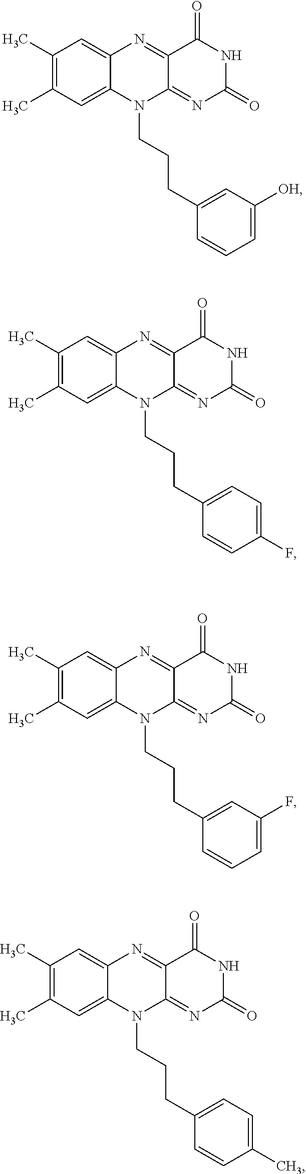 [SMILES: Cc1cc2nc3c(=O)[nH]c(=O)nc-3n(CCCc3cccc(O)c3)c2cc1C ; Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(F)c3)c2cc1C ; Cc1ccc(CCCn2c3nc(=O)[nH]c(=O)c-3nc3cc(C)c(C)cc32)cc1]

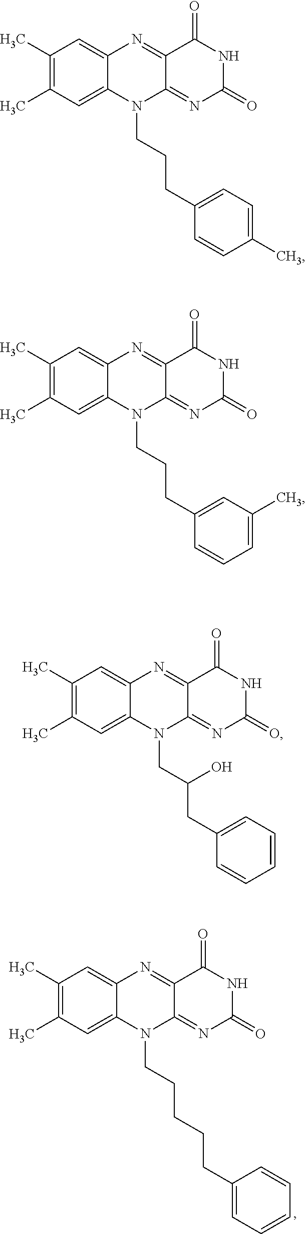 [SMILES: Cc1ccc(CCCn2c3nc(=O)[nH]c(=O)c-3nc3cc(C)c(C)cc32)cc1 ; Cc1cccc(CCCn2c3nc(=O)[nH]c(=O)c-3nc3cc(C)c(C)cc32)c1 ; Cc1cc2nc3c(=O)[nH]c(=O)nc-3n(CC(O)Cc3ccccc3)c2cc1C ; Cc1cc2nc3c(=O)[nH]c(=O)nc-3n(CCCCCc3ccccc3)c2cc1C]

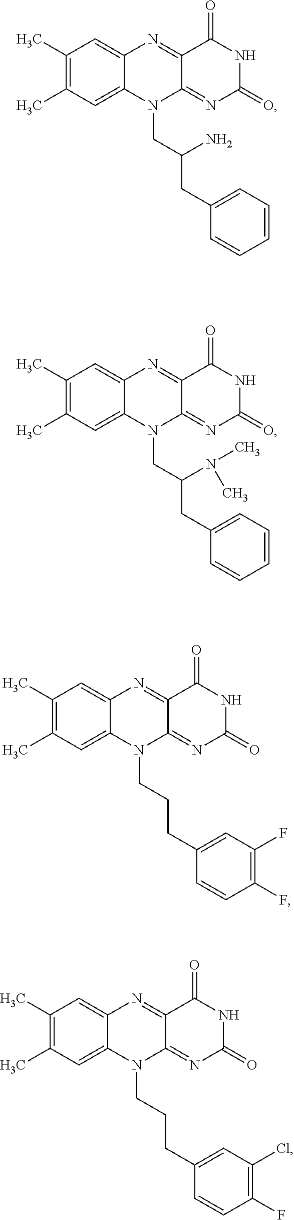 [SMILES: Cc1cc2nc3c(=O)[nH]c(=O)nc-3n(CC(N)Cc3ccccc3)c2cc1C ; Cc1cc2nc3c(=O)[nH]c(=O)nc-3n(CC(Cc3ccccc3)N(C)C)c2cc1C ; Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3ccc(F)c(Cl)c3)c2cc1C]

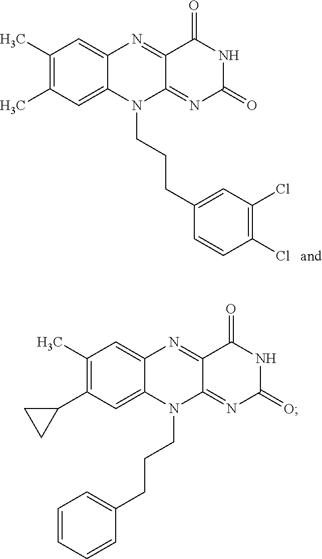 [SMILES: Cc1cc2nc3c(=O)[nH]c(=O)nc-3n(CCCc3ccc(Cl)c(Cl)c3)c2cc1C ; "and" ; Cc1cc2nc3c(=O)[nH]c(=O)nc-3n(CCCc3ccccc3)c2cc1C1CC1]

1.104 Formula 1.103, wherein the compound of Formula I is selected from any of the following:

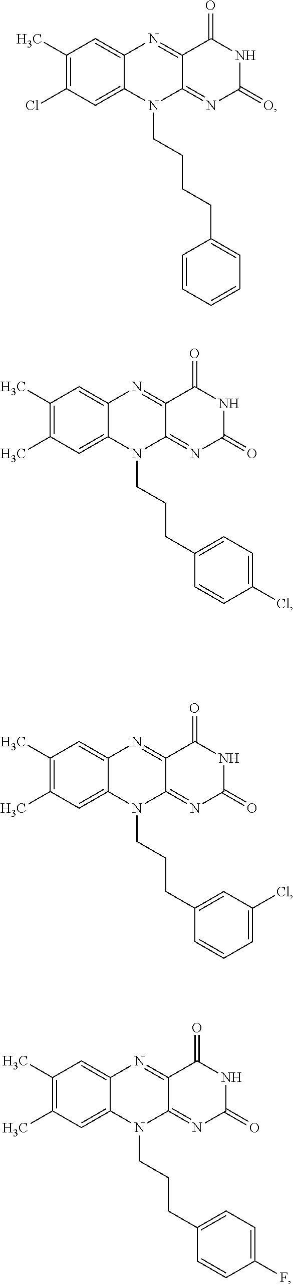 [SMILES: Cc1cc2nc3c(=O)[nH]c(=O)nc-3n(CCCCc3ccccc3)c2cc1Cl ; Cc1cc2nc3c(=O)[nH]c(=O)nc-3n(CCCc3ccc(Cl)cc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCCc3ccc(F)cc3)c2cc1C]

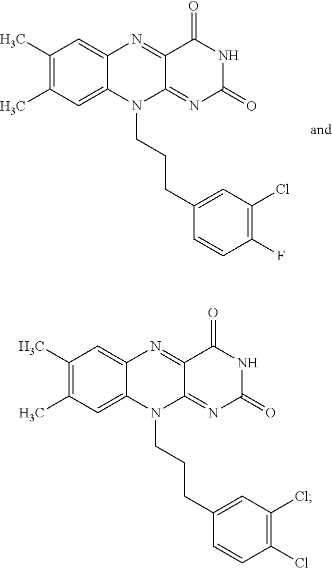 [SMILES: Cc1cc2nc3c(=O)[nH]c(=O)nc-3n(CCCc3ccc(F)c(Cl)c3)c2cc1C ; Cc1cc2nc3c(=O)[nH]c(=O)nc-3n(CCCc3ccc(Cl)c(Cl)c3)c2cc1C]

1.105 any of formulae 1.33-1.96, wherein the compound of Formula I is selected from any of the following:

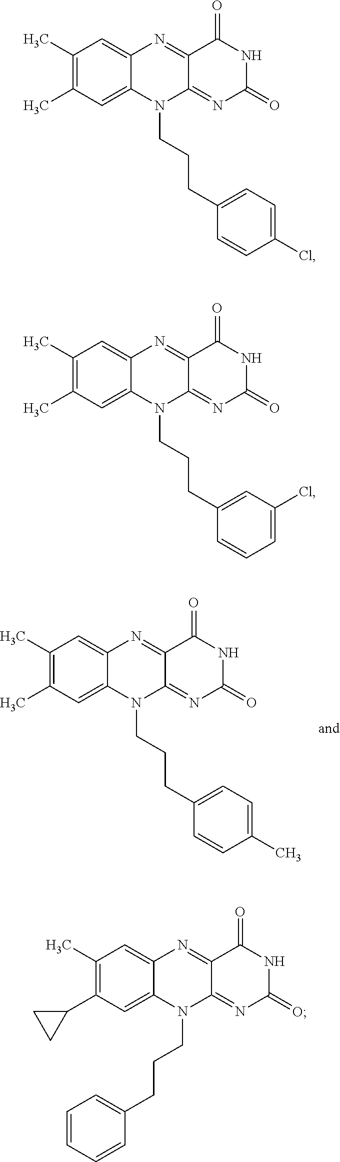

1.106 any of the preceding formulae wherein the compound of Formula I binds to FMN and/or CD3299 riboswitch, e.g., with an Imax of greater than 20%, preferably greater than 30%, more preferably greater than 40%, still more preferably greater than 50% in an assay, for example, as described in Example A, and/or has a Minimum Inhibitory Concentration (MIC) of less than or equal to 64 μg/mL, more preferably less than or equal to 32 μg/mL, for example, in an assay as described in Example B,

in free or salt form.

The invention also relates to a compound of Formula Q, wherein the substituents are as defined in any of formulae 1.33-1.106, in free or salt form (Formula 1.107).

In the first aspect, the invention provides a compound of formula P, or any of P.1-P.17, or Formula Q, or any of formulae 1.1-1.32 or 1.107, in free or salt form as hereinbefore described provided that (1) when -Alk-X-A is —CH₂CH₂-phenyl or —CH₂CH₂—O-phenyl, R₁ and R₂ are not both H; (2) when -Alk-X-A is —CH₂CH₂-(3-methoxyphenyl), then R₁ and R₂ are not both methyl; or (3) when R₂ is —C(O)OEt and -Alk-X-A is phenylethyl, then R₁ is C₁₋₆alkyl, e.g., C₁₋₄alkyl (for example, methyl, ethyl, n-propyl, isopropyl, n-prop-2-enyl, n-butyl, n-but-2-en-yl or n-hexyl), C₃₋₈cycloalkyl (e.g., cyclopropyl), or C₁₋₄alkoxy (e.g., methoxy).

In a further embodiment of the first aspect, the invention provides a compound of formula I, or any of formulae 1.33-1.106, in free or salt form as hereinbefore described provided that (1) when -Alk-X-A is —CH₂CH₂-phenyl or —CH₂CH₂—O-phenyl, R₁ and R₂ are not both H; or (2) when -Alk-X-A is —CH₂CH₂-(3-methoxyphenyl), —CH₂CH₂-(3,4,5-trimethoxyphenyl), —CH₂CH₂CH₂-(2,5-dimethoxyphenyl) or —CH₂CH₂CH₂-(2,5-dihydroxyphenyl), R₁ and R₂ are not both methyl.

In the second aspect, the invention provides a compound of Formula II″:

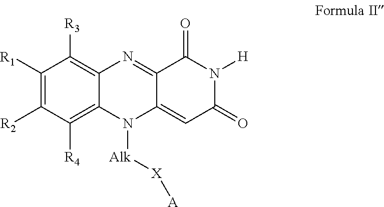

wherein:
(i) Alk is C₁₋₆alkylene (e.g., n-propylene, n-butylene, n-pentylene) optionally substituted with one or more C₁₋₆alkyl (e.g., methyl) or one hydroxy or C₁₋₄alkoxy group;
(ii) X is a single bond, —S— or —O—;
(iii) A is aryl (e.g., phenyl) or heteroaryl (e.g. pyridinyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl), or C₁₋₄alkoxy (e.g., methoxy);
(v) R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C₁₋₄alkoxy)-C₁₋₄alkyl, (2-C₁₋₄alkoxyethoxy)-C₁₋₄alkyl;
(vi) R₃ is H, C₁₋₄alkyl (e.g., methyl);
(vii) R₄ is H, C₁₋₄alkyl (e.g., methyl);
(viii) Rₐ and R_{b} are independently H, C₁₋₄alkyl (e.g., methyl) or C₃₋₈cycloalkyl (e.g., cyclopropyl, cyclopentyl),
   in free or salt form.

In the second aspect, the invention provides a compound of Formula II:

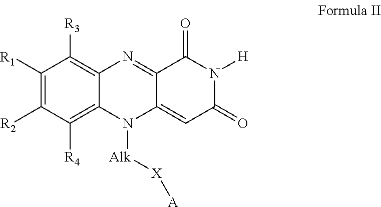

wherein:
(i) Alk is C₁₋₆alkylene (e.g., n-propylene, n-butylene, n-pentylene) optionally substituted with one hydroxy or C₁₋₄alkoxy group;
(ii) X is a single bond, —S— or —O—;
(iii) A is aryl (e.g., phenyl) or heteroaryl (e.g. pyridinyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl), or C₁₋₄alkoxy (e.g., methoxy);
(v) R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C alkoxy)-C₁₋₄alkyl, (2-C₁₋₄alkoxyethoxy)-C₁₋₄alkyl;
(vi) R₃ is H, C₁₋₄alkyl (e.g., methyl);
(vii) R₄ is H, C₁₋₄alkyl (e.g., methyl);
(viii) Rₐ and R_{b} are independently H, C₁₋₄alkyl (e.g., methyl) or C₃₋₈cycloalkyl (e.g., cyclopropyl, cyclopentyl),
   in free or salt form.

In a further embodiment of the second aspect, the invention provides a compound of the following formulae:
2.1 a compound of Formula II, wherein Alk is C₁₋₆alkylene (e.g., n-propylene, n-butylene, n-pentylene) optionally substituted with one hydroxy or C₁₋₄ alkoxy group;
2.2 a compound of Formula II or 2.1, wherein Alk is n-propylene;
2.3 a compound of Formula II or 2.1 or 2.2, wherein X is a single bond, —S— or —O—;
2.4 a compound of Formula II or any of 2.1-2.3, wherein X is a single bond, wherein said compound is represented by a compound of formula II′;

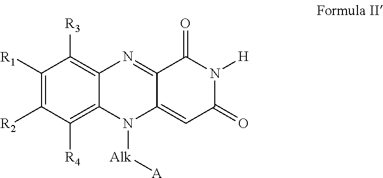

2.5 a compound of Formula II or any of 2.1-2.4, wherein A is aryl (e.g., phenyl) or heteroaryl (e.g. pyridinyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
2.6 a compound of Formula II or any of 2.1-2.5, wherein A is aryl (e.g., phenyl);
2.7 a compound of Formula II or any of 2.1-2.6, wherein A is phenyl;
2.8 a compound of Formula II or any of 2.1-2.7, wherein R₁ is H, C₁₋₄alkyl (e.g., methyl), or C₁₋₄alkoxy (e.g., methoxy);
2.9 a compound of Formula II or any of 2.1-2.8, wherein R₁ is C₁₋₄alkyl (e.g., methyl);
2.10 a compound of Formula II or any of 2.1-2.9, wherein R₁ is methyl;
2.11 a compound of Formula II or any of 2.1-2.10, wherein R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C₁₋₄alkoxy)-C₁₋₄alkyl, (2-C₁₋₄-alkoxyethoxy)-C₁₋₄alkyl;
2.12 a compound of Formula II or any of 2.1-2.10, wherein R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁alkyl-N(Rₐ)(R_{b}), (C₁₋₄alkoxy)-C₁₋₄alkyl, (2-C₁₋₄-alkoxyethoxy)-C₁₋₄alkyl;
2.13 a compound of Formula II or any of 2.1-2.11, wherein R₂ is methyl;
2.14 a compound of Formula II or any of 2.1-2.13, wherein R₃ is H, C₁₋₄alkyl (e.g., methyl);
2.15 a compound of Formula II or any of 2.1-2.14, wherein R₃ is H,
2.16 a compound of Formula II or any of 2.1-2.15, wherein R₄ is H, C₁₋₄alkyl (e.g., methyl);
2.17 a compound of Formula II or any of 2.1-2.16, wherein R₄ is H,
2.18 a compound of Formula II or any of 2.1-2.17, wherein:
   Alk is C₁₋₆alkylene (e.g., n-propylene, n-butylene, n-pentylene) optionally substituted with one hydroxy or C₁₋₄alkoxy group;
   X is a single bond, —S— or —O—;
   A is aryl (e.g., phenyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
   R₁ is C₁₋₄alkyl (e.g., methyl);
   R₂ is C₁₋₄alkyl (e.g., methyl);
   R₃ is H;
   R₄ is H,
2.19 a compound of Formula II or any of 2.1-2.18, wherein:
   Alk is C₁₋₆alkylene (e.g., n-propylene, n-butylene, n-pentylene) optionally substituted with one hydroxy or C₁₋₄alkoxy group;
   X is a single bond;
   A is aryl (e.g., phenyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
   R₁ is C₁₋₄alkyl (e.g., methyl);
   R₂ is C₁₋₄alkyl (e.g., methyl);
   R₃ is H;
   R₄ is H,
2.20 a compound of Formula II or any of 2.1-2.19, wherein:
   Alk is C₁₋₆alkylene (e.g., n-propylene, n-butylene, n-pentylene);
   X is a single bond;
   A is aryl (e.g., phenyl);
   R₁ is C₁₋₄alkyl (e.g., methyl);
   R₂ is C₁₋₄alkyl (e.g., methyl);
   R₃ is H;
   R₄ is H,
2.21 any of the preceding formulae, wherein the compound of Formula II is

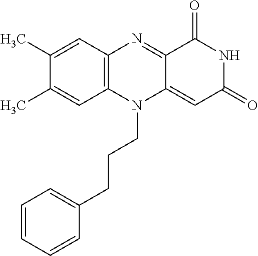 [SMILES: Cc1cc2nc3c(=O)[nH]c(=O)cc-3n(CCCc3ccccc3)c2cc1C]

2.22 any of the preceding formulae, wherein the compound of Formula II binds to FMN and/or CD3299 riboswitch, e.g., with an Imax of greater than 20%, preferably greater than 30%, more preferably greater than 40%, still more preferably greater than 50% in an assay, for example, as described in Example A, and/or has a Minimum Inhibitory Concentration (MIC) of less than or equal to 64 μg/mL, more preferably less than or equal to 32 μg/mL, still more preferably less than or equal to 16 μg/mL, most preferably less than or equal to 8 μg/mL, for example, in an assay as described in Example B,
   in free or salt form.

In a further embodiment of the second aspect, the invention provides a compound according to formula II″ wherein the substituents are as described in any one of formulae 2.1-2.22.

In the third aspect, the invention provides a pharmaceutical composition comprising a compound of Formula P, e.g., any of P.1-P.17, or Formula Q, e.g., any of formulae 1.1-1.32 or 1.107, in free or pharmaceutically acceptable salt form in admixture with a pharmaceutically acceptable diluent or carrier. In a further embodiment of the third aspect, the invention provides a pharmaceutical composition comprising a compound of Formula I, e.g., any of formulae 1.33-1.106, in free or pharmaceutically acceptable salt form in admixture with a pharmaceutically acceptable diluent or carrier. In another embodiment of the third aspect, the invention provides a pharmaceutical composition comprising a compound of Formula II″ or II, e.g., any of formulae 2.1-2.22, in free or pharmaceutically acceptable salt form in admixture with a pharmaceutically acceptable diluent or carrier.

In the fourth aspect, the invention provides a method for the treatment or prophylaxis of a bacterial infection (Method P or Q respectively) comprising administering to a subject in need thereof an effective amount of a compound of Formula P, e.g., any of P.1-P.17, or Formula Q, e.g., any of formulae 1.1-1.32 or 1.107, in free or pharmaceutically acceptable salt form. In a further embodiment of the fourth aspect, the invention provides a method for the treatment or prophylaxis of a bacterial infection (Method I) comprising administering to a subject in need thereof an effective amount of a compound of Formula I, e.g., any of formulae 1.33-1.106, in free or pharmaceutically acceptable salt form. In still another embodiment of the fourth aspect, the invention provides a method for the treatment or prophylaxis of a bacterial infection (Method II) comprising administering to a subject in need thereof an effective amount of a compound of Formula II″ or II, e.g., any of formulae 2.1-2.22, in free or pharmaceutically acceptable salt form.

In a further embodiment of the fourth aspect, Methods P, Q, I and II as hereinbefore described, are useful for the treatment or prophylaxis of a Gram-positive or Gram-negative bacterial infection (Method P-A, Method Q-A, Method I-A or Method II-A respectively). In another specific embodiment, Method P, Method Q, Method I and Method II are useful for treating a bacterial infection including, but not limited to an infection by one or more of the following bacteria: Clostridium difficile (or C. difficile), Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and/or Borrelia burgdorferi bacteria (Method P-B, Method Q-B, Method I-B or Method II-B respectively). Patients taking antibiotics, particularly those with a broad spectrum activity, are particularly vulnerable to C. difficile infection as a result of the use of antibiotics which disrupts the normal intestinal flora, leading to an overgrowth of C. difficile, causing an infection ranging from asymptomatic to severe and life-threatening condition. Various Compounds of the Invention, e.g., various compounds of Formula P, Q, I, II″ and II, particularly any compounds of any of Formulae P.17, 1.31, 1.101-1.102, 1.105 and 2.21 are particularly active against the CD3299 riboswitch and selectively inhibit C. difficile bacteria. Therefore, in a particular embodiment, Method P, Q, I and II, e.g., comprising administering a compound of any of Formulae P.17, 1.31, 1.101-1.102, 1.105 and 2.21 are particularly useful for treating an infection caused by Clostridium difficile. Further, various compounds of the invention, e.g., various compounds of Formula P, Formula Q or Formula I, particularly any compounds of Formula 1.103, 1.104 or 1.105 are also active against FMN riboswitch. Compounds which are active against FMN riboswitch are generally also active against Staphylococcus aureus and/or Clostridium difficile infections. Therefore, in particular embodiment, these compounds are especially useful for the treatment of a Staphylococcus aureus and/or Clostridium difficile infection.

In still another embodiment of the fourth aspect, Method P as hereinbefore described is useful for the treatment or prophylaxis of a disease, infection or condition selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD), comprising administering to a subject in need thereof an effective amount of a compound of Formula P, e.g., any of formulae P.1-P.17, in free or pharmaceutically acceptable salt form (Method P-D).

In yet another embodiment of the fourth aspect, Method Q as hereinbefore described is useful for the treatment or prophylaxis of a disease, infection or condition selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD), comprising administering to a subject in need thereof an effective amount of a compound of Formula Q, e.g., any of formulae 1.1-1.32 or 1.107, in free or pharmaceutically acceptable salt form (Method Q-D).

In still another embodiment of the fourth aspect, Method I as hereinbefore described is useful for the treatment or prophylaxis of a disease, infection or condition selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD), comprising administering to a subject in need thereof an effective amount of a compound of Formula I, e.g., any of formulae 1.33-1.106, in free or pharmaceutically acceptable salt form (Method I-D).

In still another embodiment of the fourth aspect, Method II as hereinbefore described is useful for the treatment or prophylaxis of a disease, infection or condition selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD), comprising administering to a subject in need thereof an effective amount of a compound of Formula II″ or II, e.g., any of formulae 2.1-2.22, in free or pharmaceutically acceptable salt form (Method II-D).

Without being bound to any particular theory, it is believed that the current invention provides methods of treating a bacterial infection via a novel mechanism, e.g., by utilizing riboswitch-ligand binding to alter gene expression. Therefore in one aspect, various compounds of the invention bind to FMN riboswitches, thereby affecting downstream riboflavin biosynthesis. In another aspect, various compounds of the invention are active against the CD3299 riboswitch, thereby affecting expression of the adjacent coding region. Compounds that are active against CD3299 and/or FMN riboswitch are particularly selective against C. difficile. As such, various Compounds of the Invention, e.g., various compounds of Formula P, e.g., various compounds of any of formulae P.1-P.17, particularly any compounds of Formule P.15-P.17, or Formula Q, e.g., various compounds of formulae 1.1-1.32 or 1.107, particularly any compounds of formulae 1.28-1.31; various compounds of Formula I, e.g., various compounds of formulae 1.33-1.106, particularly any of formulae 1.103, 1.104 or 1.105; and various compounds of Formula II″ or II, e.g., various compounds of formulae 2.1-2.22, particularly formula 2.21, in free or pharmaceutically acceptable salt form, are effective in treating an infection wherein traditional antibiotics are rendered ineffective due to drug resistance. Therefore, in a particular embodiment, the invention provides Method P, e.g., any of Methods P-A to P-D, or Method Q or any of Methods Q-A to Q-D or Method I or any of Methods I-A to I-D or Method II or any of Methods II-A to II-D as hereinbefore described wherein the infection is by an infectious agent which is resistant to a drug that is not a riboswitch ligand (Method P-E, Method Q-E, Method I-E or Method II-E respectively). In a further embodiment, various compounds of Formula P, Formula Q, Formula I, Formula II″ or Formula II, particularly any of formulae 1.103, 1.104 or 1.105 or 2.21, in free or pharmaceutically acceptable salt form are particularly useful for an infection which is resistant to one or more drugs selected from a group consisting of a penicillin, vancomycin, cephalosporin and methicillin. In a particular embodiment, the infection is a methicillin-resistant Staphylococcus aureus infection. In another embodiment, the infection to be treated in Method P, Method Q, Method I or Method II is a C. difficile infection. In a particular embodiment, various compounds of Formula P, Q, I, II″ or II, particularly any of formulae P.15-P.17, 1.28-1.30, 1.31, 1.101, 1.102, 1.105 or 2.21, in free or pharmaceutically acceptable salt form are particularly useful for the C. difficile infection which is resistant to any drug that is not a riboswitch ligand, e.g., fluoroquinolone (e.g., ciprofloxacin- and/or levofloxacin-resistant infection), metronidazole and/or vancomycin.

It will be noted that various compounds of the Invention have a low CC₅₀ value in an assay as disclosed in Example C and therefore, may have anti-metabolite activities which may interfere with DNA biosynthesis. Therefore, in one embodiment, these compounds may be useful as an anti-cancer or anti-viral agent. In another embodiment, the compounds that have a low MIC and/or a high Iₘₐₓ value in an assay as disclosed in Example B and A respectively, and a low CC₅₀ value in an assay as disclosed in Example C are used as an antibacterial, for topical administration.

In the fifth aspect, the invention provides use of a compound, or use of a pharmaceutical composition comprising a compound, of Formula P, e.g., any of P.1-P.17, in free or pharmaceutically acceptable salt form, (in the manufacture of a medicament) for the treatment or prophylaxis of an infection, e.g., a bacterial infection as described in Methods P, or any of Methods P-A through P-E. In another embodiment, the invention provides use of a compound, or use of a pharmaceutical composition comprising a compound, of Formula Q, e.g., any of formulae 1.1-1.32 or 1.107, in free or pharmaceutically acceptable salt form, (in the manufacture of a medicament) for the treatment or prophylaxis of an infection, e.g., a bacterial infection as described in Methods Q, or any of Methods Q-A through Q-E. In another embodiment of the fifth aspect, the invention provides use of a compound, or use of a pharmaceutical composition comprising a compound, of Formula I, e.g., any of formulae 1.33-1.106, in free or pharmaceutically acceptable salt form, (in the manufacture of a medicament) for the treatment or prophylaxis of an infection, e.g., a bacterial infection as described in Methods I, or any of Methods I-A through I-E. In still another embodiment of the fifth aspect, the invention provides use of a compound, or use of a pharmaceutical composition comprising a compound, of Formula II″ or II, e.g., any of formulae 2.1-2.22, in free or pharmaceutically acceptable salt form, (in the manufacture of a medicament) for the treatment or prophylaxis of an infection, e.g., a bacterial infection as described in Methods II, or any of Methods II-A through II-E.

In the sixth aspect, the invention provides use of a compound, or use of a pharmaceutical composition comprising a compound, of Formula P, e.g., any of P.1-P.17, in free or pharmaceutically acceptable salt form, (in the manufacture of a medicament) for the treatment or prophylaxis of an infection, e.g., a bacterial infection as described in Methods Q, or any of Methods Q-A through Q-E. In another embodiment, the invention provides use of a compound, or use of a pharmaceutical composition comprising a compound, of Formula Q, e.g., any of formulae 1.1-1.32 or 1.107, in free or pharmaceutically acceptable salt form, for the treatment or prophylaxis of an infection, e.g., a bacterial infection as described in Methods Q, or any of Methods Q-A through Q-E. In another embodiment of the sixth aspect, the invention provides use of a compound or use of a pharmaceutical composition comprising a compound of Formula I, e.g., any of formulae 1.33-1.106, in free or pharmaceutically acceptable salt form, for the treatment or prophylaxis of an infection, e.g., a bacterial infection as described in Methods I, or any of Methods I-A through I-E. In still another embodiment of the sixth aspect, the invention provides use of a compound or use of a pharmaceutical composition comprising a compound of Formula II″ or II, e.g., any of formulae 2.1-2.22, in free or pharmaceutically acceptable salt form, for the treatment or prophylaxis of an infection, e.g., a bacterial infection as described in Methods II, or any of Methods II-A through II-E.

In the seventh aspect, the invention provides a method for the treatment of an infection in a plant comprising administering to such plant an effective amount of a compound of Formula P, e.g., any of P.1-P.17, in free or pharmaceutically acceptable salt form. In another embodiment, the invention provides a method for the treatment of an infection in a plant comprising administering to such plant an effective amount of a compound of Formula Q, e.g., any of formulae 1.1-1.32 or 1.107, in free or pharmaceutically acceptable salt form. In another embodiment of the seventh aspect, the invention provides a method for the treatment of an infection in a plant comprising administering to such plant an effective amount of a compound of Formula I, e.g., any of formulae 1.33-1.106, in free or pharmaceutically acceptable salt form. In yet another embodiment of the seventh aspect, the invention provides a method for the treatment of an infection in a plant comprising administering to such plant an effective amount of a compound of Formula II″ or II, e.g., any of formulae 2.1-2.22, in free or pharmaceutically acceptable salt form.

In the eighth aspect, the invention provides a pharmaceutical composition comprising a compound of Formula P, e.g., any of P.1-P.17, in free or pharmaceutically acceptable salt form, for use in the treatment of any disease or condition as hereinbefore described, e.g., in any of Methods P or Methods P-A through P-E. In another embodiment, the invention also provides a pharmaceutical composition comprising a compound of Formula Q, e.g., any of formulae 1.1-1.32 or 1.107, in free or pharmaceutically acceptable salt form, for use in the treatment of any disease or condition as hereinbefore described, e.g., in any of Methods Q or Methods Q-A through Q-E. In another embodiment of the eighth aspect, the invention provides a pharmaceutical composition comprising a compound of Formula I, e.g., any of formulae 1.33-1.106, in free or pharmaceutically acceptable salt form, for use in the treatment of any disease or condition as hereinbefore described, e.g., in any of Methods I, or Methods I-A through 1-E. In another embodiment of the eighth aspect, the invention provides a pharmaceutical composition comprising a compound of Formula II″ or II, e.g., any of formulae 2.1-2.22, in free or pharmaceutically acceptable salt form, for use in the treatment of any disease or condition as hereinbefore described, e.g., in any of Methods II, or Methods II-A through II-E.

### DETAILED DESCRIPTION OF THE INVENTION

The term “riboswitch” or “riboswitches” is an art recognized term and refers to an mRNA which comprises a natural aptamer that binds target metabolite and an expression platform which changes in the RNA structure to regulate genes. The term “riboswitch ligand” refers to any compound such as a compound of Formula P, Formula Q or Formula I, e.g., various compounds of formulae P.1-P.17, formulae 1.1-1.106, or a compound of Formula II″ or II, e.g., various compounds of formulae 2.1-2.22, in free or salt form, that binds to that particular riboswitch. For example; “FMN riboswitch” refers to a riboswitch that binds a metabolite such as flavin mono-nucleotide (FMN) or other ligands such as various compound of Formula Q, particularly various compounds of Formula P, e.g., any of P.1-P.17, particularly various compounds of Formulae P.15-P.17; or various compound of Formula Q, particularly various compounds of Formulae 1.28-1.31; or various compounds of Formula I, e.g., various compounds of any of formulae 1.33-1.106, particularly compounds of formula 1.103, 1.104 or 1.105, in free or salt form, and which affects downstream FMN biosynthesis and transport proteins. Without intended to be bound by any particular theory, it is believed the binding of the ligand to its riboswitch induces a conformational change in the bacterial mRNA such that the expression of the ORF is repressed, for example, such that the expression of enzymes responsible for, e.g., riboflavin and FMN biosynthesis is repressed. This is achieved by inducing the mRNA to form (1) a terminator hairpin that halts RNA synthesis before the ORF can be synthesized or (2) a hairpin that sequesters the Shine-Dalgarno sequence and prevents the ribosome from binding to the mRNA so as to translate the ORF.

“CD3299 riboswitch” refers to a riboswitch found in C. difficile, controlling the gene designated CD3299. The 5′UTR and beginning of ORF from CD3299 gene of C. difficile 630, accession number AM180355 is as follows:

SEQ ID NO: 1:
TTACAGCTTTCTGATTTTGATAAATTTAAAACTTACCATCTAATACTAA
TAACAGGTTAATTTTATCTAATTATTATAGATTCTCATACTGTGCCTTA
TTCTATCTATAAATACAATTTAAGTGTCCATATTGAAATATTTGTATTG
TAATACAGCTGGATATTACTTAAATCCAATTGTTTCCATTATAATTTTA
TGTTAAAATAATATTACAAAATACATCTGTTTTTCTTCATAAACGGGTG
AAATTCCCTATCGGCGGTAAAAGCCCGCGAGCCTTATGGCATAATTTGG
TCATATTCCAAAGCCAACAGTAAAATCTGGATGGTAGAAGAAAATAGTA
TATGAGTACCTTTATGTAATTTTACATGAGTAATCTATACAAATCCTTC
AACTACCGTATTTATTCATGAAATTAGACACATTCAAG [symbol] [symbol] [symbol]
TTTTTTTGTTGTTTATTTTACAATTATATCGTACTT
ATAAAATCTATTAAGATTGGAGTGTTATC [symbol] [symbol] AATGGATAG
TATTGATTATCATCTGTATTGGTGTATTTATGTCTACTCTTGATGGAAG
TATACTAAATATCGCAAA

In the above depiction of the sequence, the riboswitch is highlighted in bold, and is

SEQ ID NO: 2
GTTTTTCTTCATAAACGGGTGAAATTCCCTATCGGCGGTAAAAGCC
CGCGAGCCTTATGGCATAATTTGGTCATATTCCAAAGCCAACAGTA
AAATCTGGATGGTAGAAGAAAATA

The ORF start site in the above sequence is downstream from the riboswitch and is depicted in italics and is:

SEQ ID NO: 3

[symbol]

[symbol]

The putative terminator hairpin is in bold italics and is:

SEQ ID NO: 4

[symbol]

[symbol]

[symbol]

The hairpin can form a loop having a structure as depicted in Formula 1:

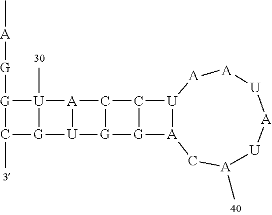

A possible antiterminator has a structure as depicted in Formula 2:

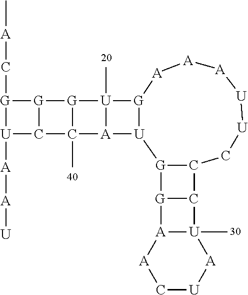

We have shown that various Compounds of the Invention, particularly compounds of Formula P.17, 1.31, 1.101 or 1.102, 1.105 or 2.21, in free or salt form, bind well to the CD3299 riboswitch and have antibacterial activity against C. difficile, provided these compounds possess physicochemical characteristics amenable to uptake into the bacteria.

The term “infection” encompasses an infection by a Gram-positive or Gram-negative bacteria. In one embodiment, the infection is by a Gram-positive bacteria. In another embodiment, the infection is by a Gram-negative bacteria. In still another embodiment, the infection is an infection by one or more bacteria selected from a group consisting of Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and/or Borrelia burgdorferi. In a further embodiment, the infection is a Clostridium difficile and/or Staphylococcus aureus infection. In a particular embodiment, the infection is an infection which is resistant to a drug which is not a riboswitch ligand. In a further aspect of this particular embodiment, the infection is an infection which is resistant to one or more drugs selected from a group consisting of penicillin, vancomycin, cephalosporin, methicillin and fluoroquinolone (e.g., ciprofloxacin- and/or levofloxacin). In a particular embodiment, the infection is a methicillin-resistant Staphylococcus aureus (MRSA) infection. In another particular embodiment, the infection is a fluoroquinolone-resistant (e.g., ciprofloxacin- and/or levofloxacin-resistant), metronidazole and/or vancomycin-resistant C. difficile infection.

The term “bacteria” or “bacterial” include, but are not limited to Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and/or Borrelia burgdorferi.

If not otherwise specified or clear from context, the following terms as used herein have the following meetings:
a. “Alkyl” as used herein is a saturated or unsaturated hydrocarbon moiety, preferably saturated, e.g., one to eight, e.g., one to six, e.g., one to four carbon atoms in length, which may be linear or branched (e.g., n-butyl or tert-butyl) unless otherwise specified, and may be optionally substituted, e.g., mono-, di-, or tri-substituted on any one of the carbon atoms, e.g., with C₁₋₄alkyl (e.g., methyl), C₁₋₄alkoxy, halogen (e.g., chloro or fluoro), haloC₁₋₄alkyl (e.g., trifluoromethyl), hydroxy, and carboxy. For example, “C₁-C₈ alkyl” denotes alkyl having 1 to 8 carbon atoms. Examples of alkyl include, but are not limited to, methyl, ethyl, n-propyl, i-propyl, n-butyl, i-butyl, sec-butyl, t-butyl, 3-methylpentyl, 4-methylpentyl, n-pentyl, n-hexyl and n-heptyl. Wherein the alkyl group is unsaturated or partially saturated, it is denoted as “alkenyl” or “alkynyl”. Therefore, n-prop-2-en-1-Y1 is intended to be —CH₂—CH═CH₂.
b. For the avoidance of doubt, the term “alkylene” is intended to denote an alkyl group bridging between two substituents (e.g., between the flavin core structure and another substituent, for example —X-A). Therefore C₁₋₄alkylene, e.g., methylene, ethylene, n-propylene and n-butylene are intended to represent —CH₂—, —CH₂CH₂—, —CH₂CH₂CH₂— and —CH₂CH₂CH₂CH₂— respectively. Wherein the alkylene group is unsaturated or partially saturated, it is denoted as “alkenylene” or “alkynylene”. Therefore, n-but-2-enylene is intended to be —CH₂—CH═CHCH₂—.
c. “Aryl” as used herein is a monocyclic or polycyclic aromatic hydrocarbon, preferably phenyl, optionally substituted, e.g., with C₁₋₄alkyl (e.g., methyl), C₁₋₄alkoxy, halogen (e.g., chloro or fluoro), haloC₁₋₄alkyl (e.g., trifluoromethyl), hydroxy, carboxy, or an additional aryl or heteroaryl.
d. “Cycloalkyl” refers to a saturated or unsaturated nonaromatic hydrocarbon moiety, preferably saturated, preferably comprising three to eight carbon atoms, at least some of which form a nonaromatic mono- or bicyclic, or bridged cyclic structure.
e. “Heterocycloalkyl” refers to a cycloalkyl as defined above wherein at least one of the carbon atoms is replaced with a heteroatom selected from N, O, S. Therefore, “C₃₋₈heterocycloalkyl” or “heteroC₃₋₈cycloalkyl” refers to a 3- to 8-membered non-aromatic ring system containing at least one heteroatom selected from N, O and S.
f. Wherein the substituent is connected via an alkyl group, e.g., —C₀₋₄alkyl-C₃₋₈cycloalkyl or aryl-C₁₋₄alkyl, it is understood that the alkyl group may be saturated or unsaturated or linear or branched. Wherein the substituent is connected via the C₀-alkyl, it is understood that the alkyl is not present and the connectivity is directly to the next substituent. For example, wherein the substituent is —C₀alkyl-C₃₋₈cycloalkyl, it is understood that the alkyl group is not present and the cycloalkyl (e.g., cyclopropyl) is directly connected.

The Compounds of the Invention or any of the compounds disclosed herein (e.g. a compound of Formula P or any of P.1-P, e.g., any of P.1-P.17, or Formula Q or Formula I, e.g., any of formulae 1.1-1.107 or a compound of Formula II″ or II, e.g., any of formulae 2.1-2.22), may exist in free or salt, e.g., as acid addition salts, or prodrug form. An acid-addition salt of a compound of the invention which is sufficiently basic, for example, an acid-addition salt with, for example, an inorganic or organic acid, for example hydrochloric, hydrobromic, sulphuric, phosphoric, acid acetic, trifluoroacetic, citric, maleic acid, toluene sulfonic, propionic, succinic, glycolic, stearic, lactic, malic, tartaric, citric, ascorbic, pamoic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. In addition a salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine. In a particular embodiment, the salt of the compound of the invention is a trifluoroacetic or hydrochloric acid addition salt. In another embodiment, the salt of the compound of the invention is an acetic acid addition salt.

In this specification, unless otherwise indicated, language such as Compounds of the Invention is to be understood as embracing the compounds disclosed herein, such as a compound of Formula P, e.g., any of P.1-P.17, or Formula Q or Formula I, e.g., any of formulae 1.1-1.106, or a compound of Formula II″ or II, e.g., any of formulae 2.1-2.22, in any form, for example free or acid addition salt or prodrug form, or where the compounds contain acidic substituents, in base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention, and are therefore also included.

The Compounds of the Invention may comprise one or more chiral carbon atoms. The compounds thus exist in individual isomeric, e.g., enantiomeric or diasteriomeric form or as mixtures of individual forms, e.g., racemic/diastereomeric mixtures. Any isomer may be present in which the asymmetric center is in the (R)—, (S)—, or (R,S)— configuration. The invention is to be understood as embracing both individual optically active isomers as well as mixtures (e.g., racemic/diasteromeric mixtures) thereof. Accordingly, the Compound of the Invention may be a racemic mixture or it may be predominantly, e.g., in pure, or substantially pure, isomeric form, e.g., greater than 70% enantiomeric excess (“ee”), preferably greater than 80% ee, more preferably greater than 90% ee, most preferably greater than 95% ee. The purification of said isomers and the separation of said isomeric mixtures may be accomplished by standard techniques known in the art (e.g., column chromatography, preparative TLC, preparative HPLC, simulated moving bed and the like).

Geometric isomers by nature of substituents about a double bond or a ring may be present in cis (=Z-) or trans (=E-) form, and both isomeric forms are encompassed within the scope of this invention.

As will be appreciated by those skilled in the art, the Compounds of the Invention may exhibit keto-enol tautomerization. Therefore, the invention as defined in the present invention is to be understood as embracing both the structures as setforth herewith and their tautomeric forms.

It is also intended that the Compounds of the Invention encompass their stable isotopes. For example, the hydrogen atom at a certain position on the Compounds of the Invention may be replaced with deuterium. It is expected that the activity of compounds comprising such isotopes would be retained and/or it may have altered pharmacokinetic or pharmacodynamic properties. In addition to therapeutic use, compounds comprising such isotopes and having altered pharmacokinetic or pharmacodynamic properties would also have utility for measuring pharmacokinetics of the non-isotopic analogs.

Compounds of the Invention may in some cases also exist in prodrug form. The term “prodrug” is an art recognized term and refers to a drug precursors prior to administration, but generate or release the active metabolite in vivo following administration, via some chemical or physiological process. For example, when the Compounds of the Invention (e.g., a compound of Formula P, Formula Q or Formula I, e.g., any of formulae P.1-P.17, 1.1-1.106, or a compound of Formula II″ or II, e.g., any of formulae 2.1-2.22) contain a hydroxy group, these substituents may be esterified to form physiologically hydrolysable and acceptable esters (e.g., acyl esters, e.g., CH₃C(O)—O— Compound). As used herein, “physiologically hydrolysable and acceptable esters” means esters of Compounds of the Invention which are hydrolysable under physiological conditions to yield hydroxy on the one hand and acid, e.g., carboxylic acid on the other (e.g., Drug-O—C(O)—CH₃→Drug-OH+CH₃COOH), which are themselves physiologically tolerable at doses to be administered. Similarly, wherein the compounds of the invention contain an amine group, prodrug of such amine, e.g., amino acid, carbamic acid ester, amide prodrugs may also exist wherein the prodrug is cleaved to release the active amine metabolite in vivo following administration. Further details of amine prodrugs may may be found in Jeffrey P. Krise and Reza Oliyai, Biotechnology: Pharmaceutical Aspects, Prodrugs, Volume 5, Part 3, pages 801-831, the contents of which are herein incorporated by reference in their entirety. As will be appreciated, the term thus embraces conventional pharmaceutical prodrug forms.

Methods of using Compounds of the Invention

The Compounds of the Invention are useful for the treatment of an infection, particularly an infection by bacteria including but not limited to Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and/or Borrelia burgdorferi bacteria. In a particular embodiment, the bacteria is selected from any one of the following: Clostridium difficile and Staphylococcus aureus.

The invention therefore provides methods of treatment of any one or more of the following conditions: anthrax infection, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD); comprising administering an effective amount of a compound of Formula P, e.g., any of P.1-P.17, or Formula Q, e.g., any of formulae 1.1-1.32 or 1.107, Formula I, e.g., any of formulae 1.33-1.106, or a compound of Formula II″ or II, e.g., any of formulae 2.1-2.22, in free or pharmaceutically acceptable salt form, to a subject in need thereof.

The words “treatment” and “treating” are to be understood accordingly as embracing prophylaxis and treatment or amelioration of symptoms of disease as well as treatment of the cause of the disease. In one particular embodiment, the invention encompasses prophylaxis of symptoms of disease or cause of the disease. In another particular embodiment, the invention encompasses treatment or amelioration of symptoms of disease or cause of the disease.

The term “subject” as used herein encompasses human and/or non-human (e.g., animal).

Dosages employed in practicing the present invention will of course vary depending, e.g. on the particular disease or condition to be treated, the particular Compound of the Invention used, the mode of administration, and the therapy desired. Administration of a therapeutically active amount of the therapeutic compositions is defined as an amount effective, at dosages and for periods of time necessary to achieve the desired result. For example, a therapeutically effective amount of a Compound of the Invention reactive with at least a portion of the FMN or the CD3299 riboswitch may vary according to factors such as the disease state, age, sex, and weight of the individual, and the ability of the compound to elicit a desired response in the individual. Dosage regiment may be adjusted to provide the optimum therapeutic response. For example, several divided doses may be administered daily or the dose may be proportionally reduced as indicated by the exigencies of the therapeutic situation. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 1000 mg, conveniently administered once, or in divided doses 2 to 4 times, daily or in sustained release form. Unit dosage forms for oral administration thus for example may comprise from about 0.2 to 75 mg, 250 mg, 1000 mg, e.g. from about 0.2 or 2.0 to 50, 75, 100, 250, 500, 750 or 1000 mg of a Compound of the Invention, together with a pharmaceutically acceptable diluent or carrier therefor.

Pharmaceutical compositions comprising the Compounds of the Invention may be prepared using conventional diluents or excipients and techniques known in the galenic art. Thus oral dosage forms may include tablets, capsules, solutions, suspensions, spray-dried dispersions [e.g. Eudragit L100] and the like. The term “pharmaceutically acceptable carrier” as used herein is intended to include diluents such as saline and aqueous buffer solutions. The Compounds of the Invention may be administered in a convenient manner such as by injection such as subcutaneous, intravenous, by oral administration, inhalation, transdermal application, intravaginal application, topical application, intranasal, sublingual or rectal administration. Depending on the route of administration, the active compound may be coated in a material to protect the compound from the degradation by enzymes, acids and other natural conditions that may inactivate the compound. In one embodiment, the compound may be orally administered. In another embodiment, the compound is administered via topical application.

In certain embodiment, the Compounds of the Invention may be administered alone or in conjunction, e.g., at or about the same time or simultaneously and separately or simultaneously in an admixture, with another agent, e.g., an agent to facilitate entry or permeability of the Compounds of the Invention into the cell, e.g., an antimicrobial cationic peptide. Antimicrobial cationic peptides include peptides which contain (1) a disulfide-bonded β-sheet peptides; (2) amphipathic α-helical peptides; (3) extended peptides; or (4) loop-structured peptides. Examples of cationic peptide include but are not limited to defensins, cecropins, melittins, magainins, indolicidins, bactenecin and protegrins. Other examples of antimicrobial cationic peptides include but are not limited to human neutrophil defensin-1 (HNP-1), platelet microbicidal protein-1 (tPMP), inhibitors of DNA gyrase or protein synthesis, CP26, CP29, CP11CN, CP10A, Bac2A-NH₂ as disclosed in Friedrich et al., Antimicrob. Agents Chemother. (2000) 44(8):2086, the contents of which are hereby incorporated by reference in its entirety. Further examples of antibacterial cationic peptides include but are not limited to polymyxin e.g., polymixin B, polymyxin E or polymyxin nonapeptide. Therefore, in another embodiment, the Compounds of the Invention may be administered in conjunction with polymyxin, e.g., polymixin B, polymyxin E or polymyxin nonapeptide, preferably polymyxin B.

In still another embodiment, the Compounds of the Invention may be administered alone or in conjunction, e.g., at or about the same-time, simultaneously and separately, or simultaneously in an admixture, with other antimicrobial agents, e.g., other antifungal or other systemic antibacterial (bactericidal or bacteriostatic) agents. Examples of bacterial agents include agents which inhibit bacterial cell wall synthesis (e.g., penicillins, cephalosporins, carbapenems, vancomycin), agents which damage cytoplasmic membrane (e.g., polymixins as discussed above), agents which modify the synthesis or metabolism of nucleic acids (e.g., quinolones, rifampin, nitrofurantoin), agents which inhibit protein synthesis (aminoglycosides, tetracyclines, chloramphenicol, erythomycin, clindamycin), agents which interfer with the folate synthesis (e.g., folate-inhibitors), agents which modify energy metabolism (e.g., sulfonamides, trimethoprim) and/or other antibiotics (beta-lactam antibiotic, beta-lactamase inhibitors). Specific anti-infective agents, particularly antibacterial and antifungal agents, are discussed in Remington: The Science and Practice of Pharmacy, Chapter 90, pp. 1626-1684 (21^{st} Ed., Lippincott Williams & Wilkins 2005), the contents of which are hereby incorporated by reference.

### Methods of Making the Compounds of the Invention:

The compounds of the Invention, e.g., compound of Formula P, Formula Q or Formula I, e.g., any of formulae P.1-P.17, 1.1-1.106, or a compound of Formula II″ or II, e.g., any of formulae 2.1-2.22, in free or salt form may be made using the methods as described and exemplified herein and by methods similar thereto and by methods known in the chemical art. Such methods include, but not limited to, those described below. In the description of the synthetic methods described herein, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. Therefore, at times, the reaction may require to be run at elevated temperature or for a longer or shorter period of time. It is understood by one skilled in the art of organic synthesis that functionality present on various portions of the molecule must be compatible with the reagents and reactions proposed. If not commercially available, starting materials for these processes may be made by procedures, which are selected from the chemical art using techniques which are similar or analogous to the synthesis of known compounds. All references cited herein are hereby incorporated by reference in their entirety.

The synthetic methods for the Compounds of the Invention are illustrated below either in the generic synthetic scheme and/or in the specific Examples, which methods are claimed individually and/or collectively. The significances for the substituents are as set forth above in Formula P, e.g., any of P.1-P.17, Formula Q or Formula I, e.g., any of formulae 1.1-1.106, or Formula II″ or II, e.g., any of formulae 2.1-2.22, unless otherwise indicated.

Generally, the compounds of Formula P, Formula Q or Formula I, e.g., any of formulae P.1-P.17 or 1.1-1.106, may be prepared as follows: (1) reacting a nitro aniline, Int-A′, with an A-X-Alk-L, Int-B′, wherein L is a leaving group, e.g., a halide, e.g., bromide, to provide Int-E′, or by (2) reacting Int-C′ with an A-X-Alk-amine, Int-D′, wherein X in this instance is a single bond, to provide Int-E′. The resulting Int-E′ may be converted to Int-F′ for example, by catalytic hydrogenation, e.g., by reacting Int-E′ with a metal, e.g., Raney Nickel, in the presence of hydrogen gas in a solvent such as ethanol to provide diamine, Int-F′. Int-F′ may react with pyrimidine-2,4,5,6(1H,3H)-tetrone in the presence of boric acid and acetic acid to obtain a compound of Formula P, Formula Q or Formula I. This preparation may be summarized in the following reaction scheme:

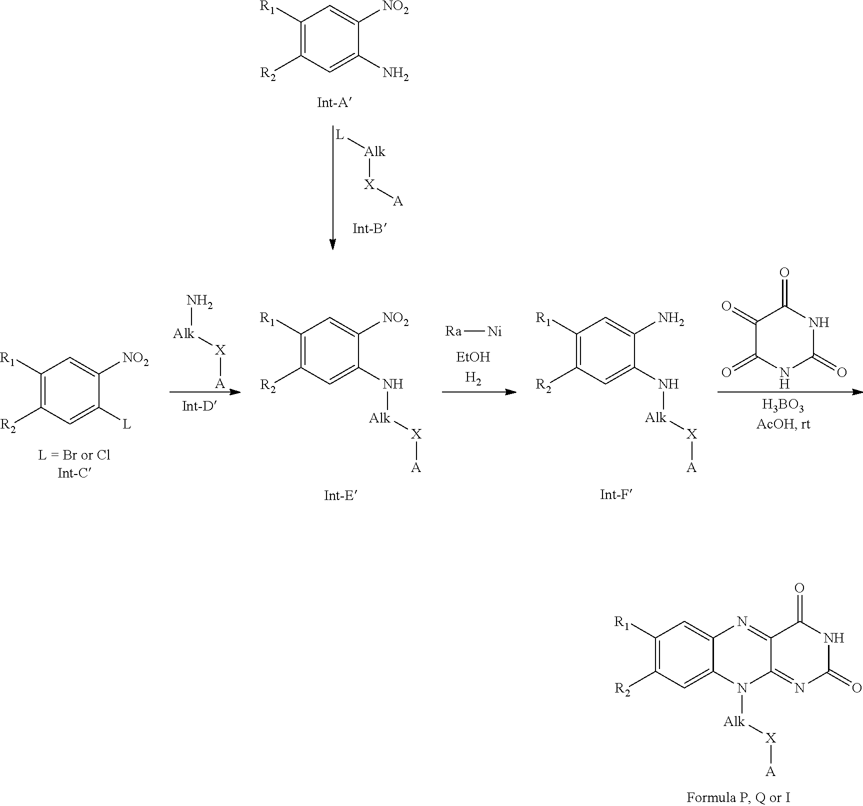

Wherein R₂ of the compounds of Formula P, Formula Q or Formula I is —C₁alkyl-N(Rₐ) (R_{b}), e.g., —CH₂—N(CH₃)₂, this compound may be prepared by halogenating the compounds of Formula P, Formula Q or Formula I, wherein R₂ is e.g., a methyl group, for example by reacting bromine with the compounds of Formula Q or Formula I, wherein R₂ is methyl, optionally in the presence of a catalyst such as azobisisobutyronitrile (AIBN). The resulting intermediate, Int-G′, may then react with an amine, HN(Rₐ)(R_{b}), e.g. HN(CH₃)₂, to provide a Compound of Formula P, Formula Q or Formula I, wherein R₂ is —C₁alkyl-N(Rₐ)(R_{b}), e.g., —CH₂—N(CH₃)₂. These preparations may be summarized in the following reaction scheme:

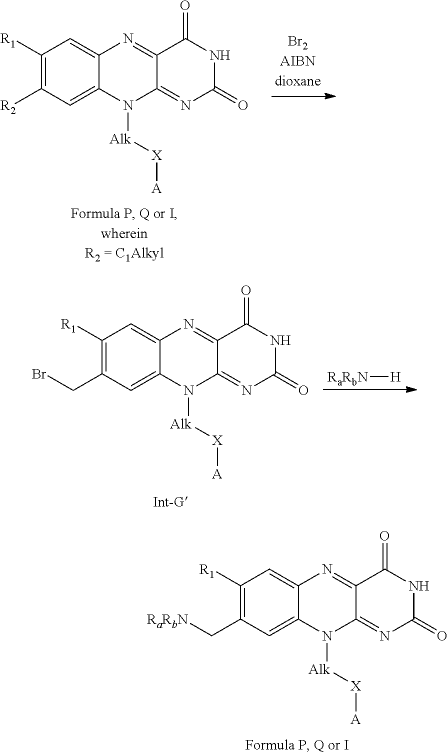

Generally, the compounds of Formula II″ or II, e.g., any of formulae 2.1-2.22 may be prepared by reacting Intermediate-5 (Int-5) with ammonia in a pressure tube. Int-5 may be prepared by reacting Intermediate-4 (Int-4) with diethyl 2-bromo-3-oxopentanedioate in the presence of a base, e.g., cesium carbonate, in a solvent, for example, a mixture of dimethylformamide (DMF) and methylene chloride (CH₂Cl₂). Int-4 may be may be prepared by converting Intermediate-3 (Int-3) to Int-4, for example, by catalytic hydrogenation, e.g., by reacting Int-3 with a metal, e.g., Raney-Nickel, and hydrogen gas in a solvent such as ethanol. In turn, Int-3 may be prepared by reacting Intermediate-1 (Int-1) with NH₂-Alk-X-A (Int-2), wherein Alk, X and A are defined in Formula II or any of 2.1-2.22 to yield Int-3. Int-1 is either commercially available or may be prepared as described in any of Examples 1-16 described below. Wherein R₂ of compounds of Formula II″ or II is alkoxy, this compound may be prepared by reacting a compound of Formula II″ or II, wherein R₂ is halo, e.g., chloro, with R₂—H, e.g., methanol, in the presence of a base. The methods for preparing a compound of Formula II″ or II may be described in the reaction scheme below, wherein all substituents are defined in Formula II″ or II or any of 2.1-2.22:

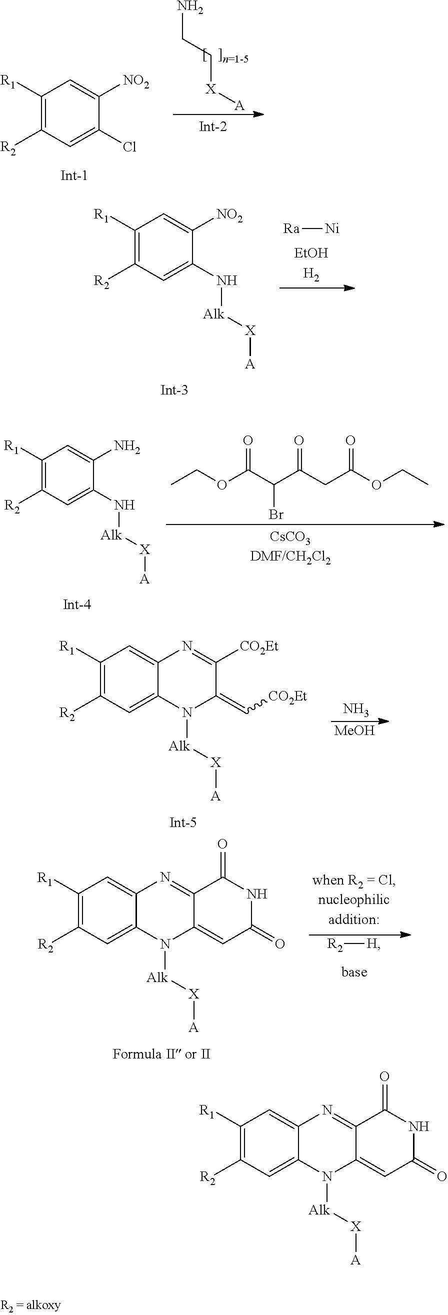

Wherein R₂ of the compounds of Formula II″ or II is (C₁₋₄alkoxy)-methyl, these compounds may be prepared by first halogenating the compound of Formula II″ or II, wherein R₂ is methyl, for example by reacting such compound with a halogen, e.g., bromine, e.g., optionally in the presence of a catalyst such as azobisisobutyronitrile (AIBN). The resulting intermediate, Int-6, may then react with a R₂—H, wherein R₂—H is e.g. methanol, in the presence of a base to provide the corresponding alkoxy-methyl product. Wherein R₂ of the compounds of Formula II is -methyl-N(Rₐ)(R_{b}), e.g., —CH₂—N(CH₃)₂, this compound may be prepared by halogenating the compounds of Formula II″ or II, wherein R₂ is e.g., a methyl group, for example by reacting bromine with the compounds of Formula II″ or II, wherein R₂ is methyl, optionally in the presence of a catalyst such as azobisisobutyronitrile (AIBN). The resulting intermediate, Int-6, may then react with an amine, HN(Rₐ)(R_{b}), e.g. HN(CH₃)₂, to provide a Compound of Formula II″ or II wherein R₂ is -methyl-N(Rₐ)(R_{b}), e.g., —CH₂—N(CH₃)₂. This preparation may be summarized in the following reaction scheme:

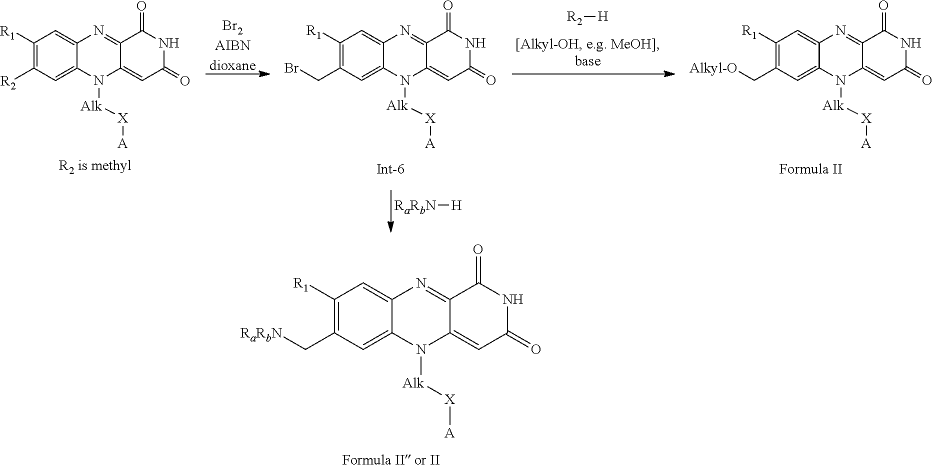

Wherein R₂ and A of the Compound of Formula P or Formula Q are linked together so as to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione, these compounds may be prepared, for example, by reacting Int-12 with benzylidene-bis(tricyclohexyl-phosphine)dichlororuthenium (i.e., first generation Grubb's catalyst), for example, in toluene at reflux. This preparation may be summarized in the following reaction schemes wherein R₆ may be H or any of the substituents allowed to substitute Aryl (e.g., phenyl) defined in A of Formula P or Formula Q:

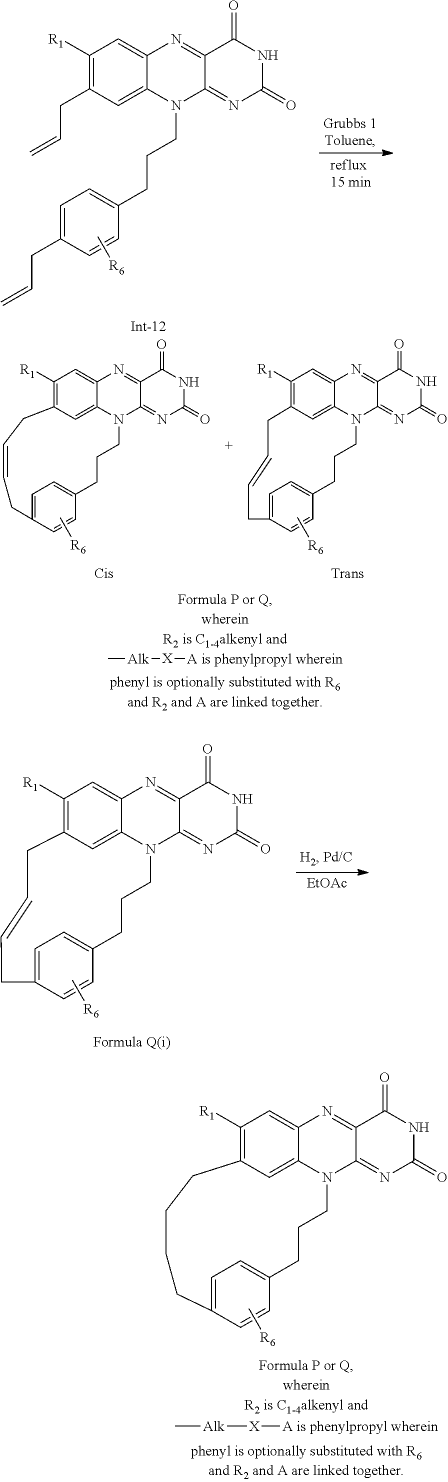

In turn, Int-12 may be prepared by first reacting Int-7 with Int-8 in the presence of a base, e.g., diisopropylethylamine to yield Int-9. Int-9 is then reacted with 6-chlorouracil in the presence of a base, e.g., diisopropylethylamine, e.g., in a solvent such as DMF to yield Int-10. Int-10 is then reacted with sodium nitrite, e.g., in a solvent such as acetic acid to yield Int-11. Int-11 is then reacted with a reducing agent, e.g., sodium hydrosulfite, e.g., in the presence of a base, e.g., triethylamine to yield Int-12. The preparation may be summaried below:

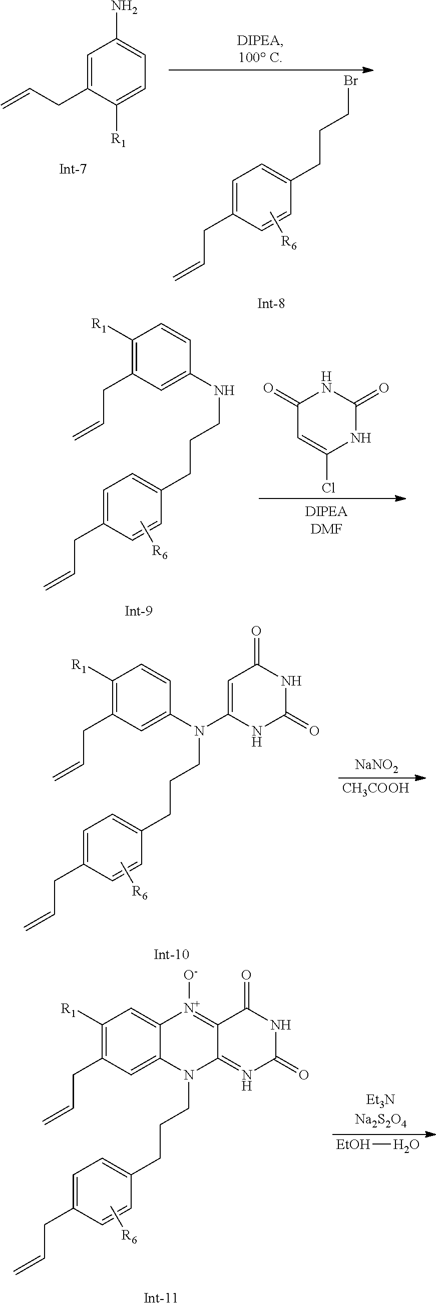

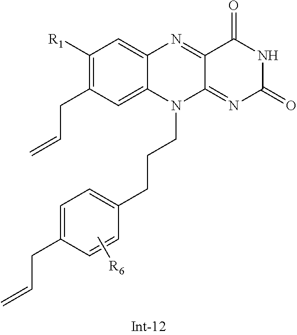

Int-7 may be prepared by reacting (R₁-substituted)-2-bromo-4-nitrobenzene with allyltributylstanane and tetrakis(triphenylphosphine)palladium(0). The resulting product is then reacted with a reducing agent, for example, zinc dust to yield Int-7. The preparation may be summaried below:

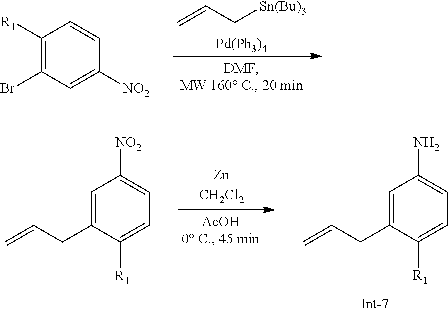

Int-8 may be prepared by as described in Examples 25 and 26 below.

### EXAMPLES

### Binding of Ligand to Riboswitch

### Example A

An in-line probing assay, as described in Regulski and Breaker, “In-line probing analysis of riboswitches”, (2008), Methods in Molecular Biology, Vol 419, pp 53-67, the contents of which are incorporated by reference in their entirety, is used to estimate the dissociation binding constants for the interaction of each of the ligands described herein with either an FMN riboswitch amplified from the genome of Bacillus subtilis or a CD3299 riboswitch amplified from Clostridium difficile. Precursor mRNA leader molecules are prepared by in vitro transcription from templates generated by PCR and [5′-³²P]-labeling using methods described previously (Regulski and Breaker, In-line probing analysis of riboswitches (2008), Methods in Molecular Biology Vol 419, pp 53-67). Approximately 5 nM of labeled RNA precursor is incubated for 41 hours at 25° C. in 20 mM MgCl₂, 50 mM Tris/HCl (pH 8.3 at 25° C.) in the presence or absence of a fixed concentration of each ligand. Binding to the FMN and CD3299 riboswitches are measured at 20 μM and 100 μM, respectively. In-line cleavage products are separated on 10% polyacrylamide gel electrophoresis (PAGE), and the resulting gel is visualized using a Molecular Dynamics Phosphorimager. The location of products bands corresponding to cleavage are identified by comparison to a partial digest of the RNA with RNase T1 (G-specific cleavage) or alkali (nonspecific cleavage).

In-line probing exploits the natural ability of RNA to self-cleave at elevated pH and metal ion concentrations (pH≈8.3, 25 mM MgCl₂) in a conformation-dependent manner. For self-cleavage to occur, the 2′-hydroxyl of the ribose must be “in-line” with the phosphate-oxygen bond of the internucleotide linkage, facilitating a S_{N}2P nucleophilic transesterification and strand cleavage. Typically, single-stranded regions of the riboswitch are dynamic in the absence of an active ligand, and the internucleotide linkages in these regions can frequently access the required in-line conformation. Binding of an active ligand to the riboswitch generally reduces the dynamics of these regions, thereby reducing the accessibility to the in-line conformation, resulting in fewer in-line cleavage events within those regions. These ligand-dependent changes in RNA cleavage can be readily detected by denaturing gel electrophoresis. The relative binding affinity of each ligand is expressed as Iₘₐₓ, wherein Iₘₐₓ represents the percent inhibition of in-line cleavage at selected internucleotide ligands in the presence of a fixed ligand concentration (20 μM for the FMN riboswitch and 100 μM for the CD3299 riboswitch) normalized to the percent inhibition in the absence of ligand and the percent inhibition in the presence of a saturation concentration of a control ligand. 100 μM FMN is used as a control ligand for estimating binding to the FMN riboswitch and 100 μM of Example 1 (which is a compound which has a high affinity against the CD3299 riboswitch) is used as a control ligand for estimating binding to the CD3299 riboswitch.

The experiments show that various Compounds of the Invention have a binding affinity to the FMN riboswitch with an Iₘₐₓ value of up to 100%, meaning that they can bind almost as well as FMN at 20 μM. In other instances, various compounds of the invention have a binding affinity to the CD3299 switch with an Iₘₐₓ value of greater than 20% compared to the control at 100 μM.

### MIC Assay

### Example B

The MIC assays are carried out in a final volume of 100 μL in 96-well clear round-bottom plates according to methods established by the Clinical Laboratory Standards Institute (CLSI). Briefly, test compound suspended in 100% DMSO (or another suitable solubilizing buffer) is added to an aliquot of media appropriate for a given pathogen to a total volume of 50 μL. This solution is serially diluted by 2-fold into successive tubes of the same media to give a range of test compound concentrations appropriate to the assay. To each dilution of test compound in media is added 50 μl of a bacterial suspension from an overnight culture growth in media appropriate to a given pathogen. Final bacterial inoculum is approximately 10⁵-10⁶ CFU/well. After growth for 18-24 hours at 37° C., the MIC is defined as the lowest concentration of antimicrobial agent that completely inhibits growth of the organism as detected by the unaided eye, relative to control for bacterial growth in the absence of added antibiotic. Ciprofloxacin is used as an antibiotic-positive control in each screening assay. Each of the bacterial cultures that are available from the American Type Culture Collection (ATCC, www.atcc.org) is identified by its ATCC number.

The experiments show that various compounds of the invention, e.g., the compounds of Formulae P.15 have a minimum inhibitory concentration (MIC) of less than 64 μg/mL, in particular instance, less than or equal to 32 μg/mL and in other instances, less than or equal to 16 μg/mL and still in other instances less than or equal to 8 μg/mL against at least one of the bacteria selected from Clostridium difficile (e.g., C. difficile MMX3581 (clinical) and C. Difficile ATCC43596)), Staphylococcus epidermidis, Staphylococcus aureus (e.g., Staphylococcus aureus ATCC29213 and Stephylococcus aureus RN4220), Streptococcus pneumoniae, Pseudomonas aeruginosa, Acinetobacter baumannii, Escherichia coli, Haemophilus influenzae, Enterococcus faecalis, Streptococcus pyogenes, MMX Streptococcus pneumoniae ATCC 49619, MMX Streptococcus pneumoniae PSSP, MMX Streptococcus pneumoniae ATCC 6301, MMX Streptococcus pyogenes ATCC 19615, MMX Haemophilus influenzae ATCC 49247, Bacillus subtilis 1A1, Staphylococcus epidermidis ATCC 12228, Enterococcus faecalis ATCC 29212, S. aureus 13709 (Smith), S. epidermidis 35984, S. aureus VL134 (MRSA), S. aureus 25923 (haze not considered growth), S. aureus NRS384, C. diff ATCC 700057 (MMX 4381), C. diff ATCC BAA-1805 (NAP1), C. diff ATCC BAA-1382 (MMX4820), C. diff ATCC 43596(MMX4822), C. diff 43255(MMX4821), B. fragilis ATCC 25285 (MMX0123), C. diff ATCC 43255, C. diff ATCC 43596, C. diff ATCC 700057, C. diff ATCC BAA-1382 and B. fragilis ATCC 25285.

All of the exemplified compounds of the invention have either an Iₘₐₓ value of greater than 20% in an assay as described in Example A (compared to at least one of the two controls) and/or a MIC of less than or equal to 64 μg/mL against at least one of the bacterial strains as described in Example B. In certain embodiment, certain compounds of the invention have either an Iₘₐₓ value of greater than 50% in an assay as described in Example A (compared to at least one of the two controls) and/or a MIC of less than or equal to 16 μg/mL, in some instances, less than or equal to 8 μg/mL against at least one of the bacterial strains as described in Example B.

### Cytotoxic Assay

### Example C

The cytotoxic effects of test compounds on HepG2 are measured with a commercially available cell viability assay kit from Promega. On day 1, HepG2 cells (˜1×10⁴ cells) are seeded into each well in 96-well plate and cultured for approximately 24 h at 37° C. in a 5% CO₂ atmosphere under saturating humidity. On day 2, test compounds and DMSO controls are added to appropriate wells to give a range of test compound concentrations appropriate to the assay. Terfenadine is also added to each plate as a positive cytotoxic control. Control wells containing medium without cell are prepared to obtain a value for background luminescence. Assay plates are then cultured for approximately 24 h at 37° C. in a 5% CO2 atmosphere under saturating humidity. On day 3, assay plates are removed from 37° C. incubator and equilibrated to 22° C. Once equilibrated, CellTiter-Glo® reagent is added to each well containing cell culture medium, followed by mixing to allow cell lysis. The CellTiter-Glo® Assay measures the number of viable cells in culture based on quantitation of the ATP present, an indicator of metabolically active cells. This assay generates a luminescent signal proportional to the amount of ATP present. The amount of ATP is directly proportional to the number of cells present in culture. After the assay plate is incubated at room temperature for approximately 10 min to stabilize luminescent signal, luminescence is recorded on PerkinElmer luminometer. CC₅₀ is defined as the concentration of test compounds in μM to result in 50% reduction in luminescence signal relative to the signal for untreated cells.

The experiments show that various compounds of the invention have a CC₅₀ value of greater than or equal to 30 μM. In some instances, various compounds of the invention have a MIC to cytotox ratio of at least 1:20.

### Synthesis of the Compounds of the Invention:

Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. NMR data is in the delta values of major diagnostic protons, given in parts per million (ppm) relative to the deuterium lock signal of the deuterated solvent utilized. Conventional abbreviations for signal shape are used. For mass spectra (MS), the lowest mass major ion is reported for molecules where isotope splitting results in multiple mass spectral peaks. Solvent mixture compositions are given as volume percentages or volume ratios. In cases where the NMR spectra are complex, only diagnostic signals are reported.

### Analytical HPLC

### Method A:

Agilent 1100 HPLC, Agilent XDB C18 50×4.6 mm 1.8 micron column, 1.5 mL/min, Solvent A-Water (0.1% TFA), Solvent B—Acetonitrile (0.07% TFA), Gradient: 5 min 95% A to 95% B; 1 min. hold; then recycle, UV Detection @ 210 and 254 nm.

### Method B:

Agilent 1100 HPLC, Agilent XDB C18 150×4.6 mm 1.8 micron column, 1.5 mL/min, Solvent A-Water (0.1% TFA), Solvent B—Acetonitrile (0.07% TFA), Gradient: 7 min 95% A to 95% B; 1 min. hold; then recycle, UV Detection @ 210 and 254 nm.

### System A:

Agilent 1100 HPLC, Agilent XDB C8 150×4.6 mm 5 micron column, 1.5 mL/min, Solvent A-Water (0.1% TFA), Solvent B—Acetonitrile (0.07% TFA), Gradient: 7 min 95% A to 95% B; 2.5 min. hold; then recycle, UV Detection @ 210 and 254 nm.

### Method C:

Agilent 1100 HPLC, Agilent XDB C18 50×4.6 mm 1.8 micron column, 1.5 mL/min.; Solvent A is water (0.1% TFA), solvent B is acetonitrile (0.07% TFA); Gradient: 7 min. 95% A to 95% B, 1 min. hold, then recycle; UV detection @ 214 and 254 nm.

### Method D:

Agilent 1100 HPLC, Agilent XDB C18 50×4.6 mm 1.8 micron column, 1.5 mL/min.; Solvent A is water (0.1% TFA), solvent B is acetonitrile (0.07% TFA); Gradient: 5 min. 95% A to 95% B, 1 min. hold, then recycle; UV detection @ 210 and 254 nm.

### Method E:

Agilent 1100 HPLC, Agilent XDB C18 150×4.6 mm 1.8 micron column, 1.5 mL/min.; Solvent A is water (0.1% TFA), solvent B is acetonitrile (0.07% TFA); Gradient: 7 min. 95% A to 95% B, 1 min. hold, then recycle; UV detection @ 210 and 254 nm.

### Method F:

Agilent 1100 HPLC, Agilent XDB C8 150×4.6 mm 5 micron column, 1.5 mL/min.; Solvent A is water (0.1% TFA), solvent B is acetonitrile (0.07% TFA); Gradient: 7 min. 95% A to 95% B, 2.5 min. hold, then recycle; UV detection @ 210 and 250 nm.

### Method G:

Agilent 1100 HPLC, Agilent XDB C18 50×4.6 mm 5 micron column, 1.5 mL/min.; Solvent A is water (0.1% TFA), solvent B is acetonitrile (0.07% TFA); Gradient: 6 min. 95% A to 95% B, 1 min. hold, then recycle; UV detection @ 210 and 250 nm.

### Preparative Reverse Phase Chromatography

### Method L:

Varian PrepStar, Phenomenex Luna(2) C18 250×21.2 mm 10 micron column, 20 mL/min, Solvent B—Water (0.1% TFA), Solvent A-Acetonitrile (0.07% TFA), Gradient: 10 min 5% A to 80% A; 5 min 80% A to 100% A; 5 min hold; then recycle, UV Detection @ 254 nm.

### Method M:

SunFire™ Prep C18 OBD™ 5 μm, 30×100 mm column. The aqueous phase is 0.1% TFA in USP water. The organic phase is acetonitrile. The elution profile is as follows: 95% aqueous (0 to 4 min); a gradient from 95% aqueous to 58% organic (4 to 14 min); hold at 58% organic (14 to 27 min); a gradient from 58% organic to 98% organic (27 to 30 min); 98% organic (30-33 min); a gradient from 98% organic to 95% aqueous (33-34 min); 95% aqueous (34-36 min).

### Method N:

Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: 100% aqueous (0 to 4 min.); a gradient from 100% aqueous to 60% organic (4 to 14 min.); hold at 60% organic (14 to 26 min.); a gradient to 95% organic (26 to 30 min.); hold at 95% organic (30 to 34 min.); equilibrate to aqueous.

### TERMS AND ABBREVIATIONS

ACN=acetonitrile,
br=broad,
t-BuOH=tert-butyl alcohol,
Cat.=catalytic,
Conc.=concentrated,
d=doublet,
DCM=dichloromethane,
DIAD=diisopropyl azodicarboxylate,
DMF=N,N-dimethylforamide,
DCM=dichloromethane
DMSO=dimethyl sulfoxide,
Et₂O=diethyl ether,
Et₃N=triethyl amine,
EtOAc=ethyl acetate,
EtOH=ethyl alcohol,
equiv.=equivalent(s),
flash chromatography; as described in Still, W. C, Kahn, M.; Mitra, A. J. Org. Chem. 1978, 43, 2923.
h=hour(s),
H₂O=water,
HCl=hydrochloric acid
hep=heptet,
HPLC=high performance liquid chromatography,
HOAc=acetic acid,
IPA=isopropyl alcohol,
K₂CO₃=potassium carbonate,
LiBH₄=lithium tetrahydroborate,
LAH=lithium tetrahydroaluminate,
m=multiplet,
min.=minute(s)
MgCl₂=magnesium chloride
MeOH=methanol,
NaHCO₃=sodium bicarbonate,
Na₂SO₄=sodium sulfate,
NH₄OH=ammonium hydroxide,
NH₄OAc=ammonium acetate,
NMR=nuclear magnetic resonance,
NMP=N-methylpyrrolidinone,
p=pentet,
rt=room temperature,
RNA=ribonucleic acid,
RNase T1=an endoribonuclease that specifically degrades single-stranded RNA at G residues,
s=singlet,
t=triplet,
TFA=trifluoroacetic acid,
THF=tetrahydrofuran,
TLC=thin layer chromatography,

### Intermediate A

### 1-Bromo-3-(2,6-difluorophenyl)propane

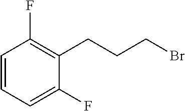 [SMILES: Fc1cccc(F)c1CCCBr]

### Step 1 Preparation of ethyl (2E)-3-(2,6-difluorophenyl)acrylate

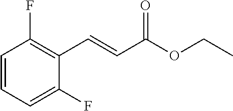

To a well-stirred solution of (carbethoxymethylidene)triphenylphosphorane (6.25 g, 17.9 mmol) in dry THF (20 mL) under nitrogen is slowly added 2,6-difluorobenzaldehyde (2.43 g, 17.1 mmol). The reaction is allowed to stir at rt for 24 h. The reaction is concentrated and the residue is subjected to silica gel chromatography (230-400 mesh, 150 g, elution with 10% ethyl acetate/hexane) to give 3.41 g (94%) of the desired product as a clear colorless oil. ¹H NMR (400 MHz, CDCl₃) δ 1.35 (3H, t), 4.23 (2H, q), 6.75 (1H, d), 6.95 (2H, m), 7.31 (1H, m), 7.78 (1H, d); MS (ESI⁺) for C₁₁H₁₀F₂O₂ m/z 213.2 (M+H)⁺, HPLC retention time: 4.91 min. (Method A).

### Step 2 Preparation of ethyl 3-(2,6-difluorophenyl)propionate

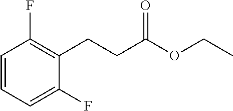

A slurry of ethyl (2E)-3-(2,6-difluorophenyl)acrylate (3.40 g, 16.0 mmol) and 10% Pd/C (100 mg, 0.9 mmol) in ethanol (50 mL, 800 mmol) is subjected to 1 atm of hydrogen gas (balloon) at rt for 24 h. The reaction mixture is filtered through Celite, the filter pad is washed with ethyl acetate and the filtrates are combined and concentrated to give 3.30 g (96%) of the desired product as clear, colorless oil. ¹H NMR (400 MHz, CDCl₃) δ 1.25 (3 H, t), 2.61 (2H, t), 3.01 (2H, t), 4.14 (2H, q), 6.86 (2H, m), 7.16 (1H, m); MS (ESI⁺) for C₁₁H₁₂F₂O₂ m/z 215.2 (M+H)⁺.

### Step 3 Preparation of 3-(2,6-difluorophenyl)propan-1-ol

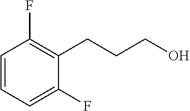 [SMILES: OCCCc1c(F)cccc1F]

A slurry of LAH (250 mg, 6.6 mmol) in dry THF (20 mL) is stirred at 0° C. under nitrogen and a solution of ethyl 3-(2,6-difluorophenyl)propionate (1.2 g, 5.6 mmol) in THF (5 mL) is added slowly. The reaction is allowed to warm to rt and stirred overnight. The reaction is cooled to 0° C. and a saturated solution of potassium sodium tartrate (5 mL) is added carefully. The mixture is then stirred at rt for 4 h, diluted with ethyl acetate and filtered through Celite. The salts are washed with ethyl acetate and the filtrate is concentrated. The residue is subjected to silica gel chromatography (230-400 mesh, 150 g, elution with 20% ethyl acetate/hexane) to give 673 mg (70%) of the desired product as a clear colorless oil. ¹H NMR (400 MHz, CDCl₃) δ 1.42 (1H, t), 1.87 (2H, m), 2.79 (2H, t), 3.68 (2H, q), 6.86 (2H, m), 7.15 (1H, m); HPLC retention time: 4.91 min. (Method A).

### Step 4 Preparation of 1-bromo-3-(2,6-difluorophenyl)propane

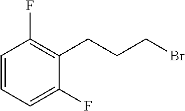 [SMILES: Fc1cccc(F)c1CCCBr]

A solution of 3-(2,6-difluorophenyl)propan-1-ol (670 mg, 0.0039 mol) and triphenylphosphine dibromide (1.72 g, 0.00409 mol) in DCM (20 mL) is stirred at rt under nitrogen for 24 h. The reaction is concentrated and the residue is subjected to silica gel chromatography (230-400 mesh, 50 g, elution with 10% ethyl acetate/hexane) to give 631 mg (69%) of the desired product as an oil. ¹H NMR (400 MHz, CDCl₃) δ 2.17 (2H, m), 2.84 (2H, t), 3.43 (2H, t), 6.87 (2H, m), 7.17 (1H, m); HPLC retention time: 5.60 min. (Method A).

### Intermediate B

### 3-(4-Chlorophenyl)propan-1-ol

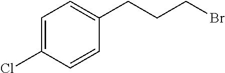 [SMILES: Clc1ccc(CCCBr)cc1]

### Step 1 Preparation of 3-(4-chlorophenyl)propan-1-ol

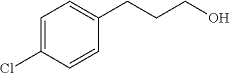 [SMILES: OCCCc1ccc(Cl)cc1]

A slurry of LAH (2.10 g, 55.2 mmol) in dry THF (250 mL) is stirred at 0° C. under nitrogen and a solution of 3-(4-chlorophenyl)propionic acid (10.2 g, 55.2 mmol) in THF (10 mL) is added slowly. The reaction is allowed to warm to rt and stirred overnight. The reaction is cooled at 0° C. and a saturated solution of potassium sodium tartrate (20 mL) is added carefully. The mixture is then stirred at it for 4 h, diluted with ethyl acetate and filtered through Celite. The salts are washed with ethyl acetate and the filtrate is concentrated to give 9.20 g (97%) of desired product as clear colorless oil. ¹H NMR (400 MHz, CDCl₃) δ 1.89 (2H, m), 2.71 (2H, m), 3.69 (2H, t), 7.15 (2H, d), 7.27 (2H, d); HPLC retention time: 3.44 min. (Method A).

### Step 2 Preparation of 1-(3-bromopropyl)-4-chlorobenzene

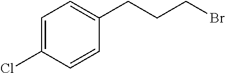 [SMILES: Clc1ccc(CCCBr)cc1]

A solution of triphenylphosphine (7.42 g, 28.3 mmol) in DCM (200 mL) is cooled at 0° C. and a solution of bromine (1.46 mL, 28.3 mmol) in DCM (40 mL) is added slowly over a period of 30 min. A solution of 3-(4-chlorophenyl)propan-1-ol (4.6 g, 27 mmol) in DCM (20 mL) is then added and the reaction is allowed to warm to it and stir for 24 h. The reaction mixture is then transferred to a separatory funnel; washed with saturated sodium bicarbonate, water and brine; and the organics are dried with anhydrous sodium sulfate and concentrated. The residue is subjected to silica gel chromatography (230-400 mesh, 350 g, elution with 7.5% ethyl acetate/hexane) to give 6.15 g (97%) of the desired product as clear colorless oil. ¹H NMR (400 MHz, CDCl₃) δ 2.16 (2H, m), 2.77 (2H, t), 3.41 (2 H, t), 7.15 (2H, d), 7.27 (2H, d); HPLC retention time: 5.11 min. (Method A).

### Intermediate C

### 1-(3-bromopropyl)-1H-pyrrole

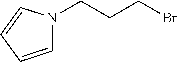

To a cooled (0-5° C.) solution 3-(1H-pyrrol-1-yl)propan-1-ol (800 mg, 6.39 mmol) in CH₂Cl₂ (30 mL) is added triphenylphosphine dibromide (3.091 g, 7.03 mmol) with stirring. After 10 min, the ice bath is removed and the mixture is stirred an additional 3 h at rt. Water is added and the mixture is diluted with CH₂Cl₂. The layers are separated and the organic layer is washed with brine, dried (anhydrous sodium sulfate), filtered and concentrated at reduced pressure. The residue is purified by flash chromatography (230-400 mesh, hexane/ethyl acetate (5%) containing 0.1% isopropanol as eluant) to afford 630 mg (52%) of the desired product as a clear oil. ¹H NMR (400 MHz, CDCl₃) δ 2.82 (p, 2 H), 3.33 (t, 2H), 4.10 (t, 2H), 6.18 (m, 2H), 6.70 (m, 2H); HPLC retention time: 3.91 min. (Method G).

### Intermediate D

### 1-(3-Bromopropyl)-1H-imidazole

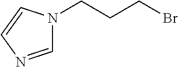

### Step 1 Preparation of methyl 3-(1H-imidazol-1-yl)propanoate

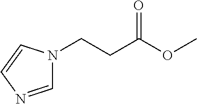

To a solution of 1H-imidazole (1.000 g, 14.7 mmol) in acetonitrile (20 mL) in a pressure tube is added methyl acrylate (2.65 mL, 29.4 mmol). The tube is sealed and heated at 80° C. Additional methyl acrylate (1.32 mL, 14.7 mmol) is added after 8 h and 12 h, respectively. After 17 h, volatiles are removed at reduced pressure and the residue is dissolved in ethyl acetate. The solution is washed with brine, dried (anhydrous sodium sulfate), filtered and concentrated at reduced pressure to afford 2.13 g (94%) of the desired product as oil. ¹H NMR (400 MHz, CDCl₃) δ 2.80 (t, 2H), 3.71 (s, 3H), 4.29 (t, 2H), 6.94 (s, 1H), 7.06 (s, 1H), 7.52 (s, 1H); MS (ESI⁺) for C₇H₁₀N₂O₂ m/z 155.2 (M+H)⁺.

### Step 2 Preparation of 3-(1H-imidazol-1-yl)propan-1-ol

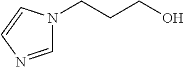

To a flask containing lithium aluminum hydride (379 mg, 9.99 mmol) is slowly added tetrahydrofuran (8 mL). The mixture is stirred for 10 min. at rt then cooled (0-5° C.). A solution of methyl 3-(1H-imidazol-1-yl)propanoate (770 mg, 4.99 mmol) in THF (3 mL) is added drop wise and the mixture is stirred an additional 5 min. at 0-5° C. The mixture is heated to 70° C. for 3 h. The mixture is cooled to rt and with vigorous stirring the reaction is quenched by the sequential addition of water (0.38 mL), 15% aqueous NaOH (0.38 mL), and water (1.14 mL). The solids are removed by filtration through a pad of Celite and the filtrate is dried (anhydrous sodium sulfate), filtered and concentrated at reduced pressure. Purification of the residue by flash chromatography (230-400 mesh, CH₂Cl₂/methanol (3-5%) as eluant) afforded 554 mg (88%) of the desired product as an oil. ¹H NMR (400 MHz, CDCl₃) δ 2.02 (p, 2H), 3.63 (t, 2H), 4.13 (t, 2H), 6.95 (s, 1H), 7.07 (s, 1H), 7.49 (s, 1H); MS (ESI+) for C₆H₁₀N₂O m/z 127.1 (M+H)⁺.

### Step 3 Preparation of 1-(3-bromopropyl)-1H-imidazole

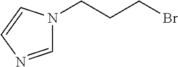

To a 0-5° C. solution 3-(1H-imidazol-1-yl)propan-1-ol (300 mg, 2.38 mmol) in CH₂Cl₂ (10 mL) is added triphenylphosphine dibromide (1.150 g, 2.62 mmol) with stirring. After 10 min., the ice bath is removed and the mixture is stirred an additional 3 h at rt. Water is added and the reaction mixture is diluted with CH₂Cl₂. The layers are separated and the organic layer is washed with saturated, aqueous sodium bicarbonate, brine, dried (anhydrous sodium sulfate), filtered and partially concentrated at reduced pressure to an approximate volume of 3 mL. This solution is used immediately in the next step. ¹H NMR (400 MHz, CDCl₃) δ 2.29 (p, 2H), 2.33 (t, 2H), 4.18 (t, 2H), 6.95 (s, 1H), 7.09 (s, 1H), 7.54 (s, 1H).

### Intermediate E

### 4-(3-bromopropyl)-2-chloro-1-fluorobenzene

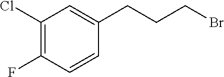 [SMILES: Fc1ccc(CCCBr)cc1Cl]

### Step 1 Preparation of ethyl (2E)-3-(3-chloro-4-fluorophenyl)acrylate

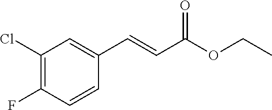

A solution of 3-chloro-4-fluorobenzaldehyde (1.0 g, 6.3 mmol) and (carbethoxymethylidene)triphenylphosphorane (2.42 g, 6.94 mmol) in dry tetrahydrofuran (25 mL, 310 mmol) is stirred at rt under nitrogen for 24 h. The mixture is concentrated and the residue is subjected to silica gel chromatography (230-400 mesh, 150 g, elution with 20% ethyl acetate/hexane) to give 1.35 g (94%) of desired product as a white solid. ¹H NMR (400 MHz, CDCl₃) δ 7.54-7.64 (m, 2H), 7.41 (m, 1H), 7.18 (t, 1H), 6.39 (d, 1H), 4.29 (q, 2H), 1.36 (t, 3H).

### Step 2 Preparation of ethyl 3-(3-chloro-4-fluorophenyl)propanoate

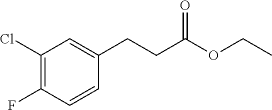

Sodium tetrahydroborate (0.993 g, 26.2 mmol) is added in 4 equal portions over 30 minutes to a mixture of ethyl (2E)-3-(3-chloro-4-fluorophenyl)acrylate (1.00 g, 4.37 mmol) and cuprous monochloride (0.649 g, 6.56 mmol) in 20 mL of MeOH which is cooled in an ice bath under N₂. The resulting mixture is stirred with ice bath cooling for 30 minutes. The reaction is quenched by addition of 20 mL of 0.5 N HCl. The MeOH is evaporated and the mixture is extracted with 3×20 mL of CH₂Cl₂. Drying of the combined organic layers over Na₂SO₄ and evaporation gives 0.90 g (89%) of desired product as a clear oil. ¹H NMR (400 MHz, CDCl₃) δ 7.16-7.19 (m, 1H), 6.98 (d, 1H), 6.93-7.04 (m, 1H), 4.05 (q, 2H), 2.83 (t, 2H), 2.52 (t, 2H), 1.16 (t, 3H).

### Step 3 Preparation of 3-(3-chloro-4-fluorophenyl)propan-1-ol

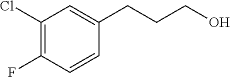 [SMILES: OCCCc1ccc(F)c(Cl)c1]

Ethyl 3-(3-chloro-4-fluorophenyl)propanoate (0.900 g, 3.90 mmol) is added to a suspension of lithium tetrahydroaluminate (0.148 g, 3.90 Mmol) in 20 mL of Et₂O which is cooled in an ice bath under N₂. After 1 h, water is added (0.15 mL), followed by 0.15 mL of 15% NaOH and 0.45 mL of water. The mixture is stirred at rt for 30 min and the solid is removed by filtration. Drying of the filtrate over Na₂SO₄ and evaporation gives 0.61 g (83%) of desired product as a clear oil. ¹H NMR (400 MHz, CDCl₃) δ 7.16 (d, 1 H), 6.97-6.99 (m, 2H), 3.55-3.65 (m, 2H), 2.61 (t, 2H), 1.75-1.85 (m, 2H), 1.24 (br s, 1H).

### Step 4 Preparation of 4-(3-bromopropyl)-2-chloro-1-fluorobenzene

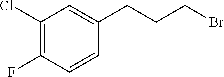 [SMILES: Fc1ccc(CCCBr)cc1Cl]

Bromine (0.250 mL, 4.85 mmol) is added to a solution of triphenylphosphine (1.27 g, 4.85 mmol) and pyridine (0.392 mL, 4.85 mmol) in 50 mL of CH₂Cl₂ which is cooled in an ice bath under N₂. Triphenylphosphine (˜0.1 g) is added until the yellow color disappears. 3-(3-chloro-4-fluorophenyl)propan-1-ol (0.610 g, 3.23 mmol) is added dropwise as a solution in 10 mL of CH₂Cl₂ and the mixture is stirred with ice bath cooling for 15 min. The ice bath is removed and the mixture is stirred at rt for 1 h. The mixture is extracted with 3×50 mL of 1.0 N HCl and 50 mL of saturated, aqueous NaHCO₃. Drying over Na₂SO₄ and evaporation of the solvent gives a white solid. The solid is suspended in 200 mL of hexane and the mixture is stirred at rt for 30 min. The solid is removed by filtration through a pad of silica gel (100 g) and the pad is eluted with 400 mL of 5% EtOAc/hexane. Evaporation of the eluate gives 0.76 g (93%) of desired product as a clear oil. ¹H NMR (400 MHz, CDCl₃) δ 7.20-7.40 (m, 1H), 7.08 (d, 2H), 3.40 (t, 2H), 2.77 (t, 2H), 2.16 (m, 2H).

### Intermediate F

### 5-(3-Bromopropyl)-3-methylisoxazole

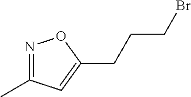

### Step 1 Preparation of 3-(3-methylisoxazol-5-yl)propan-1-ol

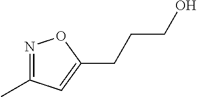

n-Butyllithium (2.5 M in hexane) (8.24 mL, 20.6 mmol) is added to a solution of 3,5-dimethylisoxazole-(2.02 mL, 20.6 mmol) in 20 mL of THF which is cooled to −78° C. under N₂. The mixture is stirred at −78° C. for 2 h. A solution of ethylene oxide (0.907 g, 20.6 mmol) in 10 mL of THF is added to the mixture at −78° C. and the mixture is stirred at −78° C. for 30 min. Saturated, aqueous NH₄Cl is added and the mixture is warmed to rt. The pH of the aqueous phase is adjusted to ˜7 with 1.0 N HCl and the THF is evaporated. The solution is extracted with 3×20 mL of CH₂Cl₂ and the combined organic layers are dried over Na₂SO₄. Evaporation of the organic layer gives 1.7 g of an oil. Residual 3,5-dimethylisoxazole is removed by drying under high vacuum at rt for 2 h to give 1.3 g (45%) of the desired product as an orange oil. ¹H NMR (400 MHz, CDCl₃) δ 5.86 (s, 1 H), 3.72 (d, 2H), 2.85 (t, 2H), 2.28 (s, 3H), 1.91-2.00 (m, 2H), 1.65 (m, 1H).

### Step 2 Preparation of 5-(3-bromopropyl)-3-methylisoxazole

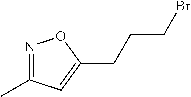

Bromine (0.109 mL, 2.12 mmol) is added to a solution of triphenylphosphine (0.557 g, 2.12 mmol) and pyridine (0.172 mL, 2.12 mmol) in 20 mL of CH₂Cl₂ which is cooled in an ice bath under N₂. Triphenylphosphine is added until the yellow color disappears. 3-(3-Methylisoxazol-5-yl)propan-1-ol (0.200 g, 1.42 mmol) is added and the mixture is stirred with ice bath cooling for 15 min. The ice bath is removed and the mixture is stirred at rt for 1 h. The mixture is extracted with 3×20 mL of 1.0 N aqueous HCl followed by 20 mL of saturated, aqueous NaHCO₃. The organic layer is dried over Na₂SO₄ and evaporation gives 0.4 g of a white solid. The solid is taken up in 20 mL of hexane and the solid is removed by filtration through a pad of silica gel (20 g). The pad is eluted with 200 mL of 50% EtOAc/hexane. Evaporation of the elutant gives 0.22 g (70%) of desired product as a clear oil. ¹H NMR (400 MHz, CDCl₃) δ 5.90 (s, 1H), 3.45 (t, 2H), 2.93 (t, 2H), 2.29 (s, 3H), 2.26 (m, 2H).

### Intermediate G

### 2-Methoxy-3-phenylpropan-1-amine

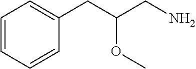

### Step 1 Preparation of 1-azido-3-phenylpropan-2-ol

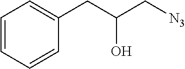 [SMILES: [N-]=[N+]=NCC(O)Cc1ccccc1]

A solution of 2-benzyloxirane (2.0 g, 15 mmol), sodium azide (1.06 g, 16.4 mmol) in DMF (10 mL) and water (2 mL) is heated at 65° C. for 18 h. The reaction is partitioned between ethyl acetate and brine (50 mL each), the layers are separated and the aqueous layer is extracted with ethyl acetate (3×25 mL). The organic layers are combined, dried with anhydrous sodium sulfate and concentrated. The residue is subjected to silica gel chromatography (230-400 mesh, 150 g, elution with 5, 10 and 20% ethyl acetate) to give 1.24 g (47%) of 1-azido-3-phenylpropan-2-ol as an oil. ¹H NMR (400 MHz, CDCl₃) δ 1.97 (1H, br s), 2.83 (2H, m), 3.31 (1H, m), 3.40 (1H, m), 4.02 (1H, m), 7.22-7.36 (5H, m); HPLC retention time: 3.23 min. (Method D).

### Step 2 Preparation of (3-azido-2-methoxypropyl)benzene

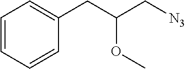

To a cold (0° C.) solution of 1-azido-3-phenylpropan-2-ol (0.354 g, 2.00 mmol) in dry THF (15 mL, 180 mmol) under nitrogen is added sodium hydride (0.0959 g, 2.40 mmol) as a solid. This mixture is stirred for 30 min. at 0° C. and methyl iodide (155 uL, 2.50 mmol) is added via syringe. The reaction mixture is then allowed to warm to room temperature and is stirred overnight. The reaction mixture is partitioned between saturated, aqueous ammonium chloride and ethyl acetate (30 mL each), the layers are separated and the aqueous layer is extracted with ethyl acetate (3×20 mL). The organic layers are combined, dried with anhydrous sodium sulfate and concentrated. The residue is subjected to silica gel chromatography (230-400 mesh, 40 g, elution with 10% ethyl acetate/hexane) to give 341 mg (89%) of (3-azido-2-methoxypropyl)benzene as a clear colorless oil. ¹H NMR (400 MHz, CDCl₃) δ 2.78 (1H, m), 2.93 (1H, dd), 3.19 (1H, m), 3.28 (1H, m), 3.42 (3H, s), 3.56 (1H, m), 7.20-7.33 (5H, m); HPLC retention time: 4.20 min. (Method D).

### Step 3 Preparation of 2-methoxy-3-phenylpropan-1-amine

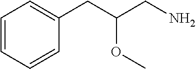

A well-stirred slurry of (3-azido-2-methoxypropyl)benzene (341 mg, 1.78 mmol) and 10% palladium on carbon (38.0 mg, 0.357 mmol) in ethanol (10 mL) is subjected to 1 atm of hydrogen gas (balloon) for 18 h. The reaction mixture is diluted with ethyl acetate (10 mL) and is filtered through Celite. The filter pad is washed with ethyl acetate (3×10 mL) and the filtrate is concentrated. The residue is subjected to silica gel chromatography (230-400 mesh, 40 g, elution with 5% MeOH (7M NH₃)/DCM) to give 146 mg (49%) of 2-methoxy-3-phenylpropan-1-amine as an oil. ¹H NMR (400 MHz, CDCl₃) δ 1.25, (2H, br s), 2.66 (1H, m), 2.77 (1H, m), 3.19 (1H, m), 2.92 (1H, dd), 3.36 (1H, m), 3.40 (3H, s), 7.20-7.33 (5H, m); MS (ESI⁺) for C₁₀H₁₅NO m/z 166.2 (M+H)⁺; HPLC retention time: 2.14 min. (Method D).

### Intermediate H

### 3-(4-Chlorophenyl)-2-isopropoxypropan-1-amine

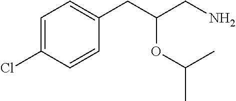

### Step 1 Preparation of 2-(4-chlorobenzyl)oxirane

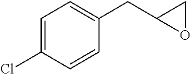 [SMILES: Clc1ccc(CC2CO2)cc1]

A well-stirred solution of 1-allyl-4-chlorobenzene (1.10 g, 7.21 mmol) in DCM (60 mL) is cooled at 0° C. and solid MCPBA (1.82 g, 7.93 mmol) is added over a five minute period. The ice bath is removed and the reaction is allowed to stir at rt for 18 h. The reaction is quenched with saturated, aqueous ammonium chloride (10 mL), partitioned between ethyl acetate and water (50 mL each), the layers are separated and the aqueous layer is extracted with ethyl acetate (3×25 mL). The organics are combined, dried with anhydrous sodium sulfate and concentrated. The residue is subjected to silica gel chromatography (230-400 mesh, 150 g, elution with 10% ethyl acetate/hexane) to give 945 mg (77%) of 2-(4-chlorobenzyl)oxirane as a clear, colorless oil. ¹H NMR (400 MHz, CDCl₃) δ 2.53 (1H, dd), 2.80 (1H, m), 2.85 (2H, m), 3.13 (1H, m), 7.19 (2H, d), 7.29 (2H, d); HPLC retention time: 3.72 min. (Method D).

### Step 2 Preparation of 3-(4-chlorophenol)-2-isopropoxypropan-1-ol

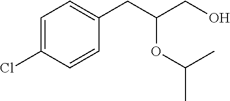

A slurry of 2-(4-chlorobenzyl)oxirane (0.970 g, 5.75 mmol) and indium trichloride (254.5 mg, 1.150 mmol) in isopropyl alcohol (12 mL) is stirred at 50° C. for 24 h. The reaction mixture is concentrated and then partitioned between DCM and water (200 mL each). The layers are separated and the aqueous layer is extracted with (3×100 mL). The organic layers are combined, dried with anhydrous sodium sulfate and concentrated. The residue is subjected to silica gel chromatography (230-400 mesh, 350 g, elution with 10,15, 20 and 25% ethyl acetate/hexane) to give 340 mg (25%) of 3-(4-chlorophenyl)-2-isopropoxypropan-1-ol as an oil. ¹H NMR (400 MHz, CDCl₃) δ 1.06 (3H, d), 1.16 (3H, d), 2.13 (1H, br s), 2.78 (2H, m), 3.45 (1H, m), 3.59 (3H, m), 7.17 (2H, d), 7.27 (2H, d); HPLC retention time: 3.74 min. (Method D).

### Step 3 Preparation of 3-(4-chlorophenyl)-2-isopropoxypropyl methanesulfonate

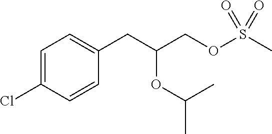 [SMILES: CC(C)OC(COS(C)(=O)=O)Cc1ccc(Cl)cc1]

A solution of 3-(4-chlorophenyl)-2-isopropoxypropan-1-ol (360 mg, 1.6 mmol) and triethylamine (263 uL, 1.89 mmol) in DCM (7.8 mL, 120 mmol) is cooled at 0° C. and methanesulfonyl chloride (146 uL, 1.89 mmol) is added slowly via syringe. The reaction mixture is stirred at 0° C. for 1 h and is allowed to warm to rt. After 2 h at rt, the reaction is quenched with saturated, aqueous ammonium chloride (2 mL). The reaction mixture is partitioned between DCM and water (50 mL each), the layers are separated and the aqueous layer is extracted with DCM (3×30 mL). The organic layers are combined, dried with anhydrous sodium sulfate and concentrated to provide 465 mg (96%) of 3-(4-chlorophenyl)-2-isopropoxypropyl methanesulfonate. ¹H NMR (400 MHz, CDCl₃) δ 0.97 (3H, d), 1.14 (3H, d), 2.79 (2H, m), 3.06 (3H, s), 3.56 (1H, m), 3.75 (1H, m), 4.09 (1 H, m), 4.17 (1H, m), 7.16 (2H, d), 7.26 (2H, d); HPLC retention time: 4.34 min. (Method D).

### Step 4 Preparation of 1-(3-azido-2-isopropoxypropyl)-4-chlorobenzene

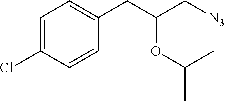 [SMILES: CC(C)OC(CN=[N+]=[N-])Cc1ccc(Cl)cc1]

To a well-stirred solution of 3-(4-chlorophenyl)-2-isopropoxypropyl methanesulfonate (0.465 g, 1.52 mmol) in MeOH (6.0 g, 190 mmol) and water (3.0 g, 170 mmol) is added sodium azide (0.319 g, 4.91 mmol). The reaction mixture is heated at 65° C. for 72 h and cooled to rt. The mixture is concentrated to remove MeOH and the residue is partitioned between DCM and brine (30 mL each). The layers are separated and the aqueous layer is extracted with DCM (3×20 mL). The organic layers are combined, dried with anhydrous sodium sulfate and concentrated. The residue is subjected to silica gel chromatography (230-400 mesh, 50 g, elution with 5% ethyl acetate/hexane) to give 254 mg (66%) of 1-(3-azido-2-isopropoxypropyl)-4-chlorobenzene as an oil. ¹H NMR (400 MHz, CDCl₃) δ 1.03 (3H, d), 1.19 (3H, d), 2.78 (2H, m), 3.20 (2H, m), 3.62 (2H, m), 7.16 (2H, d), 7.26 (2 H, d); HPLC retention time: 5.01 min. (Method D).

### Step 5 Preparation of 3-(4-chlorophenyl)-2-isopropoxypropan-1-amine

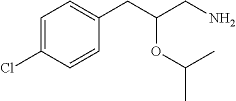

To a cold (0° C.) well-stirred solution of 1-(3-azido-2-isopropoxypropyl)-4-chlorobenzene (0.254 g, 1.00 mmol) in dry THF (5.0 mL) is added 1.00 M of trimethylphosphine in THF (1.50 mL, 1.50 mmol). After 30 min. at 0° C., water (0.5 mL) is added, the ice bath is removed and stirring is continued for 2 h. The reaction mixture is partitioned between brine and ethyl acetate (50 mL each), the layers are separated and the aqueous layer is extracted with ethyl acetate (3×25 mL). The organic layers are combined, dried with anhydrous sodium sulfate and concentrated. The residue is subjected to silica gel chromatography (230-400 mesh, 10 g, elution with 5% MeOH (7M NH₃)/DCM) to give 215 mg (94%) of 3-(4-chlorophenyl)-2-isopropoxypropan-1-amine as an oil. ¹H NMR (400 MHz, CDCl₃) δ 1.04 (3H, d), 1.18 (3H, d), 2.75 (4H, m), 3.54 (2H, m), 7.16 (2H, d), 7.26 (2H, d); MS (ESI⁺) for C₁₂H₁₈ClNO m/z 228.2 (M+H)⁺; HPLC retention time: 2.92 min (Method D).

### Intermediate I

### 4-Ethyl-5-methyl-2-nitroaniline

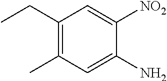

### Step 1 Preparation of N-(4-ethyl-5-methylphenyl)acetamide

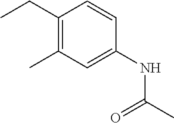

### Procedure A:

To a well-stirred solution of 4-bromo-3-methylacetanilide (5.0 g, 22 mmol) in anhydrous 1,4-dioxane (100 mL) is added [1,1′-bis(diphenylphosphino)-ferrocene]dichloropalladium(II) (0.802 g, 1.10 mmol). This mixture is sparged with nitrogen for 20 min. and diethyl zinc in hexane (0.8 M, 60.3 mL, 48.2 mmol) is added slowly via syringe. The reaction is stirred at rt under nitrogen for 15 min. and then heated at 80° C. for 2 h. The reaction is cooled to rt, diluted with ethyl acetate (100 mL), and washed with 1N HCl, water, saturated sodium bicarbonate and brine. The organic layers are dried with anhydrous sodium sulfate and concentrated. The residue is subjected to a silica gel (230-400 mesh, 50 g, elution with ethyl acetate) to give 3.80 g (98%) of N-(4-ethyl-5-methylphenyl)acetamide as a solid.

### Procedure B:

A well-stirred slurry of 4-bromo-3-methylacetanilide (1.54 g, 6.77 mmol), ethylboronic acid (1.00 g, 13.5 mmol) and Cs₂CO₃ (6.4 g, 19.6 mmol) in anhydrous 1,4-dioxane (30 mL) is sparged with dry nitrogen for 10 min. [1,1′-Bis(diphenylphosphino)ferrocene]dichloropalladium(II), complex with DCM (1:1) (0.553 g, 0.677 mmol) is added and sparging is continued for an additional 10 min. The reaction mixture is then heated at 80° C. for 2 h, cooled to rt, diluted with ethyl acetate (20 mL) and filtered through Celite. The residue is subjected to silica gel chromatography (230-400 mesh, 150 g, elution with 50% ethyl acetate/hexane) to give 850 mg (71%) of N-(4-ethyl-5-methyl-phenyl)acetamide as a solid. ¹H NMR (400 MHz, CDCl₃) δ 1.17 (3H, t), 2.16 (3H, s), 2.27 (3H, s), 2.57 (2H, q), 7.08 (1H, d), 7.24 (2H, m); MS (ESI⁺) for C₁₁H₁₅NO m/z 178.2 (M+H)⁺; HPLC retention time: 3.28 min. (Method D).

### Step 2 Preparation of N-(4-ethyl-5-methyl-2-nitrophenyl)acetamide

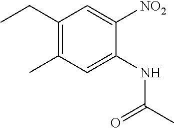 [SMILES: CCc1cc([N+](=O)[O-])c(NC(C)=O)cc1C]

To a cold (0° C.) well-stirred solution of 70% nitric acid (20 mL) and sulfuric acid (7.6 mL) is added N-(4-ethyl-5-methyl-phenyl)acetamide (4.0 g, 22 mmol) portionwise. After the addition is complete, the reaction mixture is stirred at 0° C. for 30 min. and poured onto ice (50 g). The reaction is partitioned between DCM and water (100 mL each), the layers are separated and the aqueous layer is extracted with DCM (3×50 mL). The organic layers are combined, washed with saturated, aqueous sodium bicarbonate solution, dried with anhydrous sodium sulfate and concentrated. The residue is subjected to silica gel chromatography (230-400 mesh, 350 g, elution with 10, 15 and 20% ethyl acetate/hexane) to give 3.0 g (60%) of N-(4-ethyl-5-methyl-2-nitrophenyl)acetamide as a yellow solid. ¹H NMR (400 MHz, CDCl₃) δ 1.17 (3H, t), 2.20 (3H, s), 2.31 (3H, s), 2.56 (2H, q), 7.92 (1 H, s), 8.46 (1H, s), 10.23 (1H, br s); MS (ESI⁺) for C₁₁H₁₄N₂O₃ m/z 245.2 (M+Na)⁺; HPLC retention time: 3.68 min. (Method D).

### Step 3 Preparation of 4-ethyl-5-methyl-2-nitroaniline

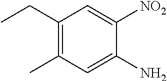

A well-stirred solution of N-(4-ethyl-5-methyl-2-nitrophenyl)acetamide (2.13 g, 9.01 mmol) in MeOH (40.0 mL) and 8.0 M of HCl (20.0 mL) is heated at 80° C. for 2 h. The reaction mixture is cooled to rt and then concentrated. The residue is suspended in water (100 mL) and the pH is adjusted to ˜10 with sodium carbonate. The mixture is then extracted with EtOAc (4×50 mL). The combined organic layers are washed with brine (1×50 mL) and then dried over anhydrous sodium sulfate. Concentration of the organic layers provided 4-ethyl-5-methyl-2-nitroaniline (1.60 g, 98%) as an orange solid. ¹H NMR (400 MHz, CDCl₃) δ 1.21 (3H, t), 2.26 (3H, s), 2.53 (2H, q), 5.91 (2H, br s), 6.59 (1H, s), 7.89 (1H, s); MS (ESI⁺) for C₉H₁₂N₂O₂ m/z 181.1 (M+H)⁺; HPLC retention time: 3.89 min. (Method D).

### Intermediate J

### Preparation of methyl 5-amino-2-methyl-4-nitrobenzoate

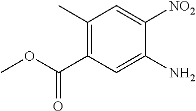

A slurry of methyl 5-acetamido-2-methyl-4-nitrobenzoate [WO 2005/080388] (0.545 g, 2.16 mmol) in 8.0 M HCl (12 mL) and MeOH (15 mL) is heated at 70° C. for 18 h. The reaction mixture is cooled to rt, carefully partitioned between saturated, aqueous sodium bicarbonate and DCM (50 mL each), the layers are separated and the aqueous layer is extracted with DCM (4×25 mL). The organic layers are combined, dried with anhydrous sodium sulfate and concentrated. The residue is subjected to silica gel chromatography (230-400 mesh, 40 g, elution with 10, 15 and 20% ethyl acetate/hexane) to give 402 mg (88%) of methyl 5-amino-2-methyl-4-nitrobenzoate as a crystalline orange solid. ¹H NMR (400 MHz, DMSO-d6) δ 2.04 (3H, s), 3.86 (3H, s), 7.89 (1H, s), 7.98 (1H, s), 10.32 (2 H, br s); MS (ESI⁺) for C₉H₁₀N₂O₄ m/z 209.1 (M−H)⁻; HPLC retention time: 3.57 min. (Method D).

### Intermediate K

### Preparation of 1-bromo-4,5-dimethyl-2-nitrobenzene

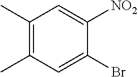

A solution of aqueous HBr (53 mL, 48 wt % in water) in water (200 mL) is added to 4,5-dimethyl-2-nitroaniline (5.00 g, 30.1 mmol) and the orange slurry is heated at 70° C. for 30 min. The mixture is cooled to −5° C. and sodium nitrite (12.9 g, 186 mmol) dissolved in water (60 mL) is added slowly. When the addition is complete, the mixture is stirred at −5° C. for 15 min. A solution of copper (I) bromide (53.95 g, 376.1 mmol) in aqueous HBr (100 mL, 48 wt % in water) is added dropwise with gas evolution at a rate to keep the reaction temperature <0° C. After addition of the copper (I) bromide solution, the mixture is heated slowly to 70° C. During heating there is gas evolution. The mixture is kept at 70° C. for 15 min. then cooled to rt and extracted with 3×200 mL of CH₂Cl₂. The combined extracts are washed with 2N sodium hydroxide and then dried with sodium sulfate. Concentration provides a brown solid which is taken up in 20 mL of CH₂Cl₂ and adsorbed onto a silica gel pad (200 g). The pad is eluted with 800 mL of 50% CH₂Cl₂/hexane and the eluate is evaporated to give 5.8 g (83%) of 1-bromo-4,5-dimethyl-2-nitrobenzene as a yellow solid. ¹H NMR (400 MHz, CDCl₃) δ 7.71 (s, 1H), 7.52 (s, 1H), 2.32 (m, 6H).

### Intermediate L

### 1-(3-Bromo-2-methylpropyl)-4-chlorobenzene

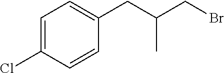

### Step 1 Preparation of ethyl 3-(4-chlorophenol)-2-methylpropanoate

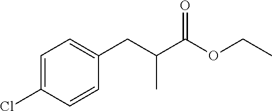

To a dry three-necked flask is added dry THF (70 mL, 900 mmol) followed by LDA in heptane (1.8 M, 15 mL, 27 mmol) and the stirred solution is cooled to −78° C. Ethyl propionate (2.82 mL, 24.5 mmol, dried by filtering through neutral alumina) is added by syringe over a 12 min., maintaining the reaction temperature below −75° C. After 20 min., a solution of 4-chlorobenzylbromide (10.07 g, 49.00 mmol) in THF (10 mL) is added dropwise by syringe (30 min.) at a rate that maintains the reaction temperature below −75° C. The reaction is allowed to warm to rt overnight and is stirred at rt for 5 days. The mixture is concentrated, diluted with EtOAc (80 mL), washed with water (3×20 mL) and concentrated to dryness giving a yellow oil. The residue is subjected to chromatography on silica gel using heptane followed by 1% EtOAc/heptane to give 3.5 g (57%) of 3-(4-chlorophenyl)-2-methylpropanoate as an oil. ¹H NMR (DMSO-d₆) δ 7.19 (m, 4H), 4.09 (q, 2H), 2.97 (m, 1H), 2.67 (m, 2H), 1.19 (m, 6H); MS (ESI−) for C₁₂H₁₅ClO₂ m/z 227.25 (M−H)⁻.

### Step 2 Preparation of 3-(4-chlorophenyl)-2-methylpropan-1-ol

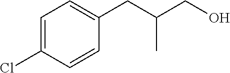

To a cold (0° C.) well-stirred solution of ethyl 3-(4-chlorophenyl)-2-methylpropanoate (2.71 g, 12.0 mmol) in dry THF (20 mL) is added LiAlH₄ (0.4537 g, 11.95 mmol) in portions over a 2 min. period. After stirring overnight, the reaction is quenched by addition of ice (10 mL) followed by water (50 mL). After stirring for 20 min., the mixture is filtered through Celite (5×20 mL EtOH rinses). The filtrate is concentrated to 60 mL and EtOAc (80 mL) is added. The layers are separated and the organic layer is washed with water (4×10 mL). The organic layer is concentrated to give 2.12 g (96%) of 3-(4-chlorophenyl)-2-methylpropan-1-ol as an oil. ¹H NMR (CDCl₃) δ 7.19 (m, 4H), 3.50 (m, 2H), 2.75 (m, 1H), 2.40 (m, 1H), 1.91 (m, 1H), 0.91 (m, 3H).

### Step 3 Preparation of 1-(3-bromo-2-methylpropyl)-4-chlorobenzene

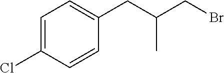

To a cold (0° C.), well-stirred solution of triphenylphosphine dibromide [freshly prepared from bromine (0.887 mL, 17.2 mmol) and triphenylphosphine (4.52 g, 17.2 mmol] in CH₂Cl₂ (60 mL) under nitrogen is added 3-(4-chlorophenyl)-2-methylpropan-1-ol (2.12 g, 11.5 mmol) as a solution in 10 mL of CH₂Cl₂. The reaction mixture is stirred with ice bath cooling for 20 min., the ice bath is removed and the reaction mixture is stirred at rt overnight. The reaction mixture is diluted with heptane (60 mL), concentrated, re-suspended in heptane and filtered. The solids are washed with heptane (4×100 mL) and the filtrates are combined and cooled at −5° C. for 2 days. The liquid is decanted off and concentrated to give 1-(3-bromo-2-methylpropyl)-4-chlorobenzene (2.6 g, 73%) as a clear colorless oil. ¹H NMR (CDCl₃) δ 7.30 (m, 4H), 3.35 (m, 2H), 2.75 (m, 1H), 2.55 (m, 1 H), 2.06 (m, 1H), 1.04 (m, 3H).

### Intermediate M

### 1-(3-Bromo-1-methylpropyl)-4-chlorobenzene

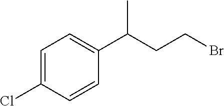

### Step 1 Preparation of 3-(4-chlorophenyl)but-3-en-1-ol

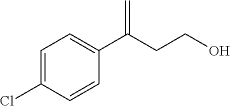

A stirred mixture of 3-bromobut-3-en-1-ol (1.10 g, 7.28 mmol), tetrakis(triphenylphosphine)palladium(0) (1.00 g, 0.865 mmol), potassium carbonate (2.01 g, 14.6 mmol), and benzene (14 mL) in water (7 mL) is sparged with N₂ for 5 min. A solution of 4-chlorophenylboronic acid (1.48 g, 9.47 mmol) in EtOH (8 mL) is added and nitrogen sparging is continued for 5 additional min. The reaction mixture is heated at 74° C. for 18 h and then cooled to rt. Aqueous hydrogen peroxide (1 mL, 35%) is added and after 30 min., the mixture is diluted with ether (80 mL). The layers are separated, the organic layer is washed with water (2×30 mL) and concentrated. The residue is chromatographed (silica gel, 10 and 15% EtOAc/heptane) to give 3-(4-chlorophenyl)but-3-en-1-ol (0.65 g, 47%) as a yellow oil. ¹H NMR (CDCl₃) δ 7.25 (m, 4H), 5.33 (s, 1H), 5.11 (s, 1H), 3.66 (t, 2H), 2.70 (t, 2H).

### Step 2 Preparation of 3-(4-chlorophenyl)butan-1-ol

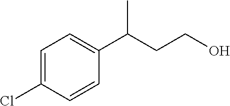

A stirred mixture of 3-(4-chlorophenyl)but-3-en-1-ol (1.060 g, 4.643 mmol), EtOAc (30 mL), zinc dibromide (0.2091 g, 0.9286 mmol) and 10% Pd/C (0.06 g, 0.06 mmol) is placed under 1 atmosphere of H₂. After 90 h, the mixture was filtered through Solka Floc® (4×5 mL EtOAc rinses) and concentrated to a pale, brown oil. The oil is washed with hot heptane (3×15 mL) and then placed under high vacuum to give 3-(4-chlorophenyl)butan-1-ol (0.847 g, 91.9%) as an oil. ¹H NMR (CDCl₃) δ 7.12 (m, 4H), 3.49 (m, 2H), 2.80 (m, 1H), 1.76 (m, 2H), 1.19 (m, 3H).

### Step 3 Preparation of 1-(3-bromo-1-methylpropyl)-4-chlorobenzene

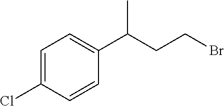

To a cold (0° C.), well-stirred solution of triphenylphosphine dibromide [freshly prepared from bromine (0.337 mL, 6.54 mmol) and triphenylphosphine (1.71 g, 6.54 mmol)] in 60 mL of CH₂Cl₂ is added 3-(4-chlorophenyl)butan-1-ol (0.847 g, 4.36 mmol) as a solution in 10 mL of CH₂Cl₂. The mixture is stirred with ice bath cooling for 20 min., the ice bath is removed and the mixture is stirred at rt for 2 h. After 2 h, the reaction mixture is diluted with heptane (60 mL), concentrated, re-suspended in heptane and filtered. The filtrate is evaporated to give 1-(3-bromo-1-methylpropyl)-4-chlorobenzene (0.719 g, 57%) as an oil. ¹HNMR (CDCl₃) δ 7.24 (m, 4H), 3.32 (m, 1H), 3.17 (m, 1H), 2:97 (m, 1H), 2.10 (m, 2 H), 1.28 (m, 3H).

### Intermediate N

### 5-(4-Methoxybutyl)-4-methyl-N-(3-phenylpropyl)benzene-1,2-diamine

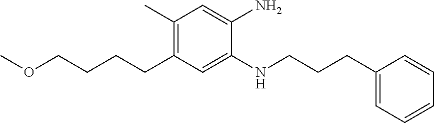 [SMILES: COCCCCc1cc(NCCCc2ccccc2)c(N)cc1C]

### Step 1 Preparation of N-(5-bromo-4-methyl-2-nitrophenyl)acetamide

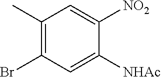

A mixture of N-(3-bromo-4-methylphenyl)acetamide (12.93 g, 56.69 mmol) (Lee et al, Adv. Syn. Cat. 2005, 347, 1921, the contents of which are incorporated by reference in their entirety) is heated in nitric acid (70% aqueous, 100 mL) at 50° C. for 2 h. The reaction is cooled to rt, poured over ice (100 g) and extracted with ethyl acetate. The combined organic extracts are washed with water, saturated, aqueous NaHCO₃, brine, dried (sodium sulfate), filtered and concentrated. The residue is purified by flash chromatography (230-400 mesh, hexanes/ethyl acetate (5 to 15% containing 0.1% isopropanol) as eluant) to afford 6.33 g (41%) of the desired product as a yellow solid. ¹H NMR (CDCl₃) δ 2.31 (s, 3H), 2.44 (s, 3H), 8.09 (s, 1H), 9.08 (s, 1H), 10.27 (br s, 1H); MS (ESI+) for C₉H₉BrN₂O₃ m/z 274.1 (M)⁺.

### Step 2 Preparation of 5-bromo-4-methyl-2-nitroaniline

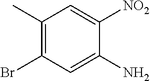

To a suspension of N-(5-bromo-4-methyl-2-nitrophenyl)acetamide (0.970 g, 3.55 mmol) in methanol (24.46 mL) is added 8.0 M aqueous HCl (19.54 mL, 156.3 mmol). The mixture is heated at 70° C. for 3 h, cooled, and the pH of the solution is adjusted to approximately 10 with solid NaHCO₃. The mixture is extracted with ethyl acetate and the combined organic extracts are washed with saturated, aqueous NaHCO₃, brine, dried (sodium sulfate), filtered and concentrated to afford 816 mg (99%) of the desired product as a yellow solid which is used without further purification. ¹H NMR (CDCl₃) δ 2.34 (s, 3 H), 5.9 (very broad exchangeable signal integrating for less than 2H), 7.10 (s, 1H), 7.99 (s, 1H). MS (ESI+) for C₇H₇BrN₂O₂ m/z 233.1 (M+H)⁺.

### Step 3 Preparation of 5-bromo-4-methyl-2-nitro-N-(3-phenylpropybaniline

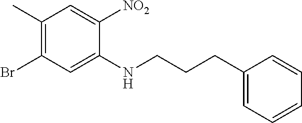 [SMILES: Cc1cc([N+](=O)[O-])c(NCCCc2ccccc2)cc1Br]

To a cooled (0-5° C.) solution of 5-bromo-4-methyl-2-nitroaniline (0.400 g, 1.73 mmol) in DMF (20 mL) is added sodium hydride (0.077 g, 1.92 mmol). After 5 min., the ice bath is removed and the mixture is stirred at ambient temperature for 30 min. A solution of 1-bromo-3-phenylpropane (0.290 mL, 1.90 mmol) in DMF (5 mL) is added and the mixture is stirred overnight at rt. Acetic acid (3 mL) is added and the mixture is concentrated. The residue is purified by flash chromatography (230-400 mesh, hexanes/ethyl acetate (0 to 1% containing 0.1% isopropanol) as eluant) to afford 0.540 g (89%) of the desired product as a yellow solid. ¹H NMR (CDCl₃) δ 2.08 (m, 2H), 2.33 (s, 3H), 2.80 (t, 2H), 3.29 (q, 2 H), 7.03 (s, 1H), 7.27 (m, 5H), 7.91 (br t, 1H), 8.03 (s, 1H).

### Step 4 Preparation of 5-allyl-4-methyl-2-nitro-N-(3-phenylpropyl)aniline

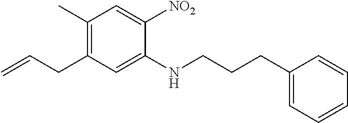 [SMILES: C=CCc1cc(NCCCc2ccccc2)c([N+](=O)[O-])cc1C]

A solution of 5-bromo-4-methyl-2-nitro-N-(3-phenylpropyl)aniline (0.540 g, 1.55 mmol) in N,N-dimethylformamide (7 mL) is sparged for 20 min. with N₂. To this solution is added bis(triphenylphosphine)palladium(II) chloride (0.043 g, 0.06 mmol). After an additional 5 min. of N₂ sparging, allyltributyltin (0.623 mL, 2.01 mmol) is added and the mixture is heated in a sealed tube for 3 h at 105° C. The mixture is diluted with water and extracted with ethyl acetate. The organic layer is washed with water, aqueous KF, brine, dried (sodium sulfate), filtered and concentrated. The residue is purified by flash chromatography (230-400 mesh, hexanes/ether (0 to 0.5%) as eluant) to afford 0.423 g (88%) of the desired product as an oil. ¹H NMR (CDCl₃) δ 2.06 (m, 2H), 2.21 (s, 3H), 2.79 (t, 3H), 3.32 (m, 4H), 5.16 (m, 2H), 5.91 (m, 1H), 6.59 (s, 1H), 7.28 (m, 5H), 7.97 (s, 1H), 8.04 (br m, 1H); MS (ESI+) for C₁₉H₂₂N₂O₂ m/z 311.4 (M+H)⁺.

### Step 5 Preparation of 5-[(2E)-4-methoxybut-2-en-1-yl]-4-methyl-2-nitro-N-(3-phenylpropyl)aniline

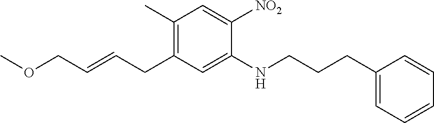 [SMILES: COC/C=C/Cc1cc(NCCCc2ccccc2)c([N+](=O)[O-])cc1C]

A solution of 5-allyl-4-methyl-2-nitro-N-(3-phenylpropyl)aniline (0.092 g, 0.30 mmol) and 10-camphorsulfonic acid (0.069 g, 0.30 mmol) in DCM (4 mL) is degassed by 3 cycles of freeze (liquid N₂), pump, thaw. To this solution is added 3-methoxyprop-1-ene (0.55 mL, 5.93 mmol) followed by Grubbs second generation catalyst (0.025 g, 0.030 mmol) under an atmosphere of nitrogen. After 18 h at rt, the mixture is concentrated and the residue purified by flash chromatography (230-400 mesh, hexanes/ether (0.2 to 0.6%) as eluant) to afford 0.061 g (58%) of the desired product as an orange oil. ¹H NMR (CDCl₃) δ 2.06 (m, 2H), 2.18 (s, 3H), 2.79 (t, 2H), 3.32 (m, 7H), 3.91 (d, 2H), 5.56 (m, 1H), 5.79 (m, 1H), 6.56 (s, 1H), 7.25 (m, 5H), 7.96 (s, 1H), 8.03 (br t, 1H); MS (ESI+) for C₂₁H₂₆N₂O₃ m/z 355.4 (M+H)⁺.

### Step 6 Preparation of 5-(4-methoxybutyl)-4-methyl-N-(3-phenylpropyl)benzene-1,2-diamine

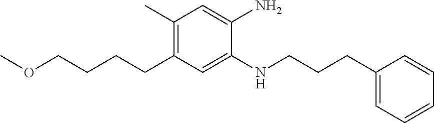 [SMILES: COCCCCc1cc(NCCCc2ccccc2)c(N)cc1C]

A slurry of 5-[(2E)-4-methoxybut-2-en-1-yl]-4-methyl-2-nitro-N-(3-phenylpropyl)aniline (0.060 g, 0.17 mmol) and Raney Nickel (ca. 20 mg) in ethanol (15 mL) is stirred at rt under 1 atm of hydrogen gas (balloon) for 18 h. The reaction mixture is diluted with ethanol (25 mL), filtered through celite and concentrated to give 0.055 g (72%) of the desired product as a tan solid. ¹H NMR (CDCl₃) δ 1.26 (m, 2H), 1.59 (m, integration obscured by water peak), 2.00 (m, 2H), 2.17 (s, 3H), 2.52 (t, 2H), 2.79 (t, 2H), 3.14 (t, 2 H), 3.35 (s, 3H), 3.41 (t, 2H), 6.42 (s, 1H), 6.53 (s, 1H), 7.28 (m, 5H); MS (ESI+) for C₂₁H₃₀N₂O m/z 327.4 (M+H)⁺.

### Intermediate 0

### 3-(4-Chlorophenyl)-2,2-dimethylpropan-1-amine

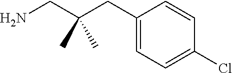

### Step 1 Preparation of ethyl 3-(4-chlorophenyl)-2,2-dimethylpropanoate

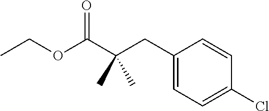

To a cooled (−78° C.) solution of N,N-diisopropylamine (1.327 mL, 9.47 mmol) in THF (6.0 mL) is added n-butyllithium (3.788 ml of a 2.500 M solution in hexane, 9.47 mmol). The solution is stirred at −78° C. for 20 min. and then allowed to warm with stirring to approximately −15° C. (iPrOH/ice) for 20 min. The pale, yellow solution is re-cooled to −78° C. and a solution of 2-methylpropanoic acid, ethyl ester (1.151 mL, 8.60 mmol) in THF (2.0 mL) is added. The solution is stirred at −78° C. for 5 min. then stirred at approximately −15° C. for an additional 20 min. The solution is re-cooled to −78° C. and a solution of 1-(bromomethyl)-4-chlorobenzene (1.946 g, 9.47 mmol) in THF (2 mL) is added. After 1 h, the dry ice bath is removed and the reaction mixture is allowed to stir at ambient temperature for 16 h. Saturated, aqueous NH₄Cl is added and the mixture is diluted with ether. The layers are separated and the organic layer washed with water, brine, dried (sodium sulfate), filtered and concentrated. The residue is purified by flash chromatography (230-400 mesh, hexanes/ether (0 to 5%) as eluant) to afford 1.80 g (87%) of the desired product as an oil. ¹H NMR (CDCl₃) δ 1.18 (s, 6H), 1.25 (t, 3H), 2.84 (s, 2 H), 4.12 (q, 2H), 7.06 (d, 2H), 7.24 (d, 2H); MS (ESI+) for C₁₃H₁₇ClO₂ m/z 241.1 (M+H)⁺.

### Step 2 Preparation of 3-(4-chlorophenyl)-2,2-dimethylpropan-1-ol

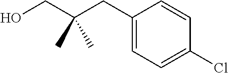

To a cooled (0-5° C.) slurry of LAH (0.175 g, 4.61 mmol) in THF (10 mL) is slowly added a solution of ethyl 3-(4-chlorophenyl)-2,2-dimethylpropanoate (1.00 g, 4.15 mmol) in THF (5 mL). The reaction mixture is allowed to warm to rt and is stirred overnight. The reaction is quenched by the sequential slow addition of water (0.175 mL), 15% aqueous NaOH (0.175 mL) followed by water (0.525 mL) with virorous stirring. The mixture is stirred for 1 h, diluted with ethyl ether and filtered through Celite. The salts are washed with ethyl ether and the combined organics are dried (sodium sulfate), filtered, and concentrated to afford 0.796 g (96%) of a pale, yellow oil which is used without further purification.

### Step 3 Preparation of 1-(3-azido-2,2-dimethylpropyl)-4-chlorobenzene

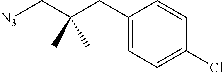

To a cooled (0-5° C.) solution of 3-(4-chlorophenyl)-2,2-dimethylpropan-1-ol (0.430 g, 2.16 mmol) in DCM (10 mL) is added triethylamine (0.528 mL, 3.79 mmol) followed by methanesulfonyl chloride (10 mL, 100 mmol) dropwise. After 1 h at 0-5° C., the reaction mixture is diluted with additional DCM and washed with ice cold water, cold 0.1 N aqueous HCl, saturated, aqueous NaHCO₃, brine, dried (sodium sulfate), filtered and concentrated to afford the crude intermediate mesylate which is used without further purification.

To a solution of the mesylate described above in DMF (6 mL) is added sodium azide (1.120 g, 17.3 mmol). The mixture is heated in a sealed tube at 125° C. for 16 h, cooled to rt and diluted with ethyl acetate. The organic layers are washed with water and brine, dried (sodium sulfate), filtered and concentrated. The residue is purified by flash chromatography (230-400 mesh, hexane/ethyl acetate (5%) as eluant) to afford 0.162 g (79%) of the desired product as an oil. ¹H NMR (CDCl₃) δ 0.93 (s, 6H), 2.55 (s, 2H), 3.07 (s, 2H), 7.08 (d, 2H), 7.27 (d, 2H).

### Step 4 Preparation of 3-(4-chlorophenyl)-2,2-dimethylpropan-1-amine

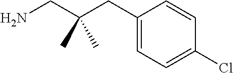

To a cold (0-5° C.) well-stirred solution of 1-(3-azido-2,2-dimethylpropyl)-4-chlorobenzene (0.220 g, 0.983 mmol) in tetrahydrofuran (4.9 mL) is added trimethylphosphine (1.48 mL of a 1.00 M solution in THF, 1.48 mmol). After 90 min. at 0-5° C., water (0.5 mL) is added, the ice bath is removed and stirring is continued for 2 h. The reaction is partitioned between brine and ethyl acetate, the layers are separated and the aqueous layer is extracted with ethyl acetate. The combined organic layers are dried (sodium sulfate), filtered and concentrated. The residue is purified by flash chromatography (230-400 mesh, DCM/2% 0.07 N methanolic ammonia as eluant) to afford 0.124 g (64%) of the desired product as an oil. ¹H NMR (CDCl₃) δ 0.84 (s, 6H), 1.33 (br s, 2H), 2.48 (s, 2H), 2.51 (s, 2H), 7.08 (d, 2H), 7.27 (d, 2H); MS (ESI+) for C₁₁H₁₆ClN m/z 198.1 (M+H)⁺.

### Intermediate P

### N-(3-{4-[(2,6-Dimethylmorpholin-4-yl)methyl]phenyl}propyl)-4,5-dimethyl-2-nitroaniline

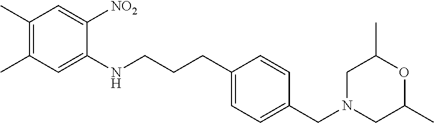

### Step 1 Preparation of ethyl 3-{4-[(2,6-dimethylmorpholin-4-yl)methyl]phenyl}propanoate

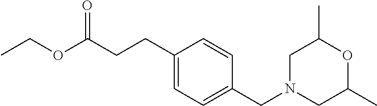 [SMILES: CCOC(=O)CCc1ccc(CN2CC(C)OC(C)C2)cc1]

To a solution of ethyl 3-(4-formylphenyl)propanoate (Najera, C., Botella, L. Tetrahedron 2005 61, 9688, the contents of which are incorporated by reference in their entirety) (1.010 g, 4.90 mmol) in 1,2-dichloroethane (20 mL) is added 2,6-dimethyl-morpholine (0.603 mL, 4.90 mmol). The solution is stirred for 30 min. and sodium triacetoxyborohydride (1.297 g, 6.12 mmol) is added. After 16 h, the mixture is diluted with ethyl acetate and the organic layers are washed with saturated, aqueous NaHCO₃, brine, dried (sodium sulfate), filtered and concentrated. The residue is purified by flash chromatography (230-400 mesh, DCM/0.07 N methanolic ammonia (0.5-1%) as eluant) to afford 0.849 g (56%) of the desired product as an oil. The product is isolated as an approximately equal mixture of diastereomers. ¹H NMR (CDCl₃) δ 1.15 (d, 3H), 1.25 (m, 6H), 1.76 (m, 1H), 2.16 (m, 1 H), 2.46 (m, 1H), 2.63 (t, 2H), 2.70 (m, 1H), 2.96 (t, 2H), 3.43 (m, 2H), 3.71 (m, 1H), 4.04 (m, 1H), 4.15 (m, 2H), 7.17 (m, 2H), 7.27 (m, 2H); MS (ESI+) for C₁₈H₂₇NO₃ m/z 306.3 (M+H)⁺.

### Step 2 Preparation of 3-{4[(2,6-dimethylmorpholin-4-yl)methyl]phenyl}propan-1-ol

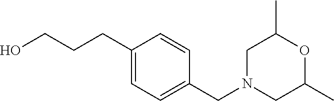 [SMILES: CC1CN(Cc2ccc(CCCO)cc2)CC(C)O1]

To a cooled (0-5° C.) slurry of LAH (0.109 g, 2.88 mmol) in THF (10 mL) is slowly added a solution of ethyl 3-{4-[(2,6-dimethylmorpholin-4-yl)methyl]phenyl}propanoate (1.840 g, 2.74 mmol) in THF (5 mL). The reaction is allowed to warm to rt and stirred overnight. The reaction is quenched by the sequential slow addition of water (0.110 mL), 15% aqueous NaOH (0.110 mL), followed by water (0.330 mL) with virorous stirring. The mixture is stirred for 1 h, diluted with ethyl ether and filtered through Celite. The salts are washed with ethyl ether and the combined organics are dried (sodium sulfate), filtered, and concentrated to afford 0.720 g (95%) of oil as an equal mixture of diastereomers which is used without further purification. ¹H NMR (CDCl₃) δ 1.16 (d, 3H), 1.25 (d, 3H), 1.91 (m, 2H), 2.16 (m, 1H), 2.46 (m, 1H), 2.72 (m, 3H), 3.43 (m, 2H), 3.70 (m, 3H), 4.03 (m, 1H), 7.20 (m, 4H); MS (ESI+) for C₁₆H₂₅NO₂ m/z 364.3 (M+H)⁺.

### Step 3 Preparation of N-(3-{4-[(2,6-dimethylmorpholin-4-ynmethyl]phenyl}propyl)-4,5-dimethyl-2-nitroaniline

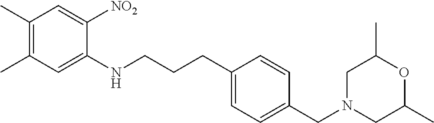

To a cooled (0-5° C.) solution of 3-{4-[(2,6-dimethylmorpholin-4-yl)methyl]phenyl}propan-1-ol (0.790 g, 3.00 mmol) in DCM (10 mL) is added triphenylphosphine dibromide (1.384 g, 3.15 mmol). The mixture is stirred at 0-5° C. for 10 min. and then at ambient temperature for an additional 3 h. The reaction is quenched by the addition of water. The mixture is diluted with additional DCM and the layers are separated. The organic layer is washed with water, then saturated, aqueous NaHCO₃, brine, dried (sodium sulfate), filtered, and partially concentrated (with a bath temperature not exceeding 25° C.) to a final volume of approximately 10 mL. DMF (10 mL) is added and the remaining DCM removed at reduced pressure at 25° C. The intermediate bromide is used without further purification in the next step.

To a cold (0-5° C.) solution of 4,5-dimethyl-2-nitroaniline (0.548 g, 3.30 mmol) in DMF (15 mL) was added sodium hydride (0.132 g, 3.30 mmol) portion wise. The mixture was stirred for 5 min. at 0-5° C. and an additional 20 min. at ambient temperature. To this mixture is added the DMF solution of bromide prepared above and the mixture is stirred at rt for 16 h. The reaction is quenched by the addition of acetic acid (3 mL). The mixture is concentrated and the residue is then concentrated twice from CHCl₃ containing 0.07 N methanolic ammonia. The residue is purified by flash chromatography (230-400 mesh, DCM/0.07 N methanolic ammonia (0.25-1%) as eluant) to provide diastereomers. Faster eluting isomer (234 mg, 19%): ¹H NMR (CDCl₃) δ 1.25 (d, 6H), 2.06 (m, 2H), 2.18 (m, 5 H), 2.25 (s, 3H), 2.47 (m, 2H), 2.77 (t, 2H), 3.35 (m, 4H), 4.03 (m, 2H), 6.57 (s, 1H), 7.18 (d, 2H), 7.28 (d, 2H), 7.93 (s, 1H), 8.04 (br t, 1H); MS (ESI+) for C₂₄H₃₃N₃O₃ m/z 412.3 (M+H)⁺. Slower eluting isomer (248 mg, 20%): ¹H NMR (CDCl₃) δ 1.15 (d, 6H), 1.81 (m, 2H), 2.09 (m, 2H), 2.19 (s, 3H), 2.26 (s, 3H), 2.78 (m, 4H), 2.32 (m, 2H), 3.53 (m, 2H), 3.75 (br m, 2H), 6.57 (s, 1H), 7.24 (m, 4H), 7.94 (s, 1H), 8.04 (br m, 1 H).

### Intermediate Q

### 4-Isopropyl-2-nitroaniline

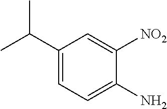

### Step 1 Preparation of 2,2,2-trifluoro-N-(4-isopropyl-2-nitrophenyl)acetamide

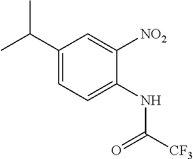 [SMILES: CC(C)c1ccc(NC(=O)C(F)(F)F)c([N+](=O)[O-])c1]

To a well-stirred solution of trifluoroacetic anhydride (50 mL) at 0° C. under nitrogen is added p-isopropylaniline (5.06 mL, 37.0 mmol) dropwise via syringe. Stirring at 0° C. is continued for 30 min. and potassium nitrate (4.12 g, 40.8 mmol) is added as a solid. The slurry is stirred at 0° C. for 1 h and is allowed to warm to rt overnight. This mixture is poured onto ice (200 g) and extracted with ethyl acetate (4×100 mL). The organic layers are combined, dried with anhydrous sodium sulfate and concentrated. The residue is subjected to silica gel chromatography (230-400 mesh, 350 g, elution with 10 and 15% ethyl acetate/hexane) to give 6.3 g (62%) of 2,2,2-trifluoro-N-(4-isopropyl-2-nitrophenyl)acetamide as a yellow solid. ¹H NMR (CDCl₃) δ 1.30 (6H, d), 3.02 (1H, hep), 7.63 (1H, dd), 8.15 (1H, d), 8.63 (1H, d), 11.28 (1H, br s); MS (ESI+) for C₁₁H₁₁F₃N₂O₃ m/z 275.4 (M−H)⁻; HPLC retention time: 4.48 min. (Method D).

### Step 2 Preparation of 4-isopropyl-2-nitroaniline

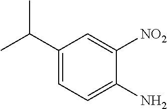

To a well-stirred solution of 2,2,2-trifluoro-N-(4-isopropyl-2-nitrophenyl)acetamide (2.1 g, 7.6 mmol) in MeOH (40 mL) is added water (20 mL) and potassium carbonate (0.5 g, 4 mmol). The reaction mixture is stirred at rt for 18 h and partitioned between ethyl acetate and brine (50 mL each). The layers are separated and the aqueous layer is extracted with ethyl acetate (3×25 mL). The organic layers are combined, dried with anhydrous sodium sulfate and concentrated. The residue is subjected to silica gel chromatography (230-400 mesh, 150 g, elution with 20 and 30% ethyl acetate/hexane) to give 1.18 g (61%) of 4-isopropyl-2-nitroaniline as an orange oil. ¹H NMR (CDCl₃) δ 1.23 (6H, d), 2.85 (1H, hep), 6.77 (1H, d), 7.28 (1H, dd), 7.96 (1H, d); MS (ESI+) for C₉H₁₂N₂O₂ m/z 181.2 (M+H)⁺; HPLC retention time: 3.97 min. (Method D).

### Intermediate R

### N-[3-(4-Chlorophenyl)-2-methylpropyl]-4-isopropyl-2-nitroaniline

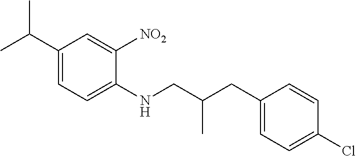 [SMILES: CC(CNc1ccc(C(C)C)cc1[N+](=O)[O-])Cc1ccc(Cl)cc1]

A vial containing 4-isopropyl-2-nitroaniline (0.274 g, 1.52 mmol), 1-(3-bromo-2-methylpropyl)-4-chlorobenzene (0.500 g, 1.76 mmol), tetra-n-butylammonium iodide (0.16 g, 0.43 mmol) and DIPEA (4 mL) is shaken at 130° C. After 18 h, additional 1-(3-bromo-2-methylpropyl)-4-chlorobenzene (0.300 g, 1.05 mmol) is added and the mixture is shaken for 6 h at 130° C., followed by overnight at 120° C. The mixture is cooled to rt and then chromatographed on silica gel using heptane to 1% EtOAc/heptane, giving the desired product as a red oil (199 mg; 25%). MS (ESI+) for C₁₉H₂₃ClN₂O₂ m/z 347.1 (M+H)⁺. HPLC retention time 6.2 min. (method D).

### Intermediate S

### 4,5-dimethyl-2-nitro-N-(2-phenylethyl)aniline

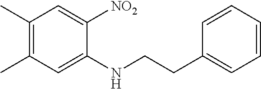 [SMILES: Cc1cc(NCCc2ccccc2)c([N+](=O)[O-])cc1C]

A mixture of 4,5-dimethyl-2-nitroaniline (0.325 g, 1.90 mmol), 1-bromo-2-phenylethane (1 mL, 7 mmol) and DIPEA (0.50 mL, 2.9 mmol) is shaken at 130° C. After 3 h, additional DIPEA (0.2 mL, 1 mmol) is added and heating is continued. After 4 h, the mixture is cooled to rt, diluted with EtOAc (50 mL)/Et₂O (100 mL), washed with water, and the organic layer is separated and concentrated. The residue is chromatographed on silica gel using 2.5% EtOAc/heptane to give desired product as a red liquid (193 mg; 37%). MS (ESI+) for C₁₆H₁₈N₂O₂ m/z 271.2 (M+H)⁺.

### Intermediate T

### 1-(3-Bromo-2-ethoxypropyl)-4-chlorobenzene

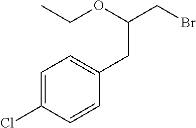

A solution of triphenylphosphine (3.6 g, 14 mmol) in DCM (200 mL) is cooled at 0° C. and a solution of bromine (0.72 mL, 14 mmol) in DCM (10 mL) is added slowly over a period of 30 min. A solution of 3-(4-chlorophenyl)-2-ethoxypropan-1-ol (2.72 g, 12.7 mmol) in DCM (20 mL) is then added and the reaction allowed to warm to rt and is stirred for 24 h. The reaction is then transferred to a separatory funnel, washed with saturated, aqeous sodium bicarbonate, water and brine. The organic layer is dried with anhydrous sodium sulfate and concentrated. The residue is subjected to silica gel chromatography (230-400 mesh, 350 g, elution with 5% ethyl acetate/hexane) to give a total of 3.18 g of the product as a clear colorless oil. ¹H NMR (400 MHz, CDCl₃) δ ppm 1.17 (3H, t), 2.85 (1H, dd), 2.96 (2H, dd), 3.41 (3H, m), 3.62 (2H, m), 7.20 (2H, d), 7.29 (2H, d); HPLC retention time: 5.18 min. (Method D).

### Intermediate U

### 4-Cyclopentyl-2-nitroaniline

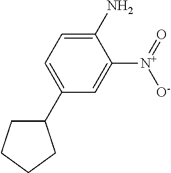 [SMILES: Nc1ccc(C2CCCC2)cc1[N+](=O)[O-]]

To a well-stirred solution of trifluoroacetic anhydride (20 mL, 200 mmol) at 0° C. under nitrogen is added 4-cyclopentylaniline (2.50 g, 15.5 mmol) dropwise via syringe. Stirring at 0° C. is continued for 30 min. The cyclopentylaniline does not dissolve. CH₂Cl₂ (20 mL) is added and the mixture is stirred at 0° C. for 30 min. Potassium nitrate (1.96 g, 19.4 mmol) is added as a solid. The slurry is stirred at 0° C. for 1 h and at rt for 4 h. The mixture is poured onto ice (200 g) and stirred at rt for 0.5 hour. The solid is collected by filtration and washed with water (3×200 mL). Air drying at rt for 0.5 hr gives a red solid. The solid is taken up in MeOH (300 mL) and potassium carbonate (3.21 g, 23.2 mmol) is added. The resulting slurry is stirred at rt for 4 h. The MeOH is evaporated and the residue is partitioned between 200 mL of CH₂Cl₂ and 200 mL of water. The water layer is extracted with 2×200 mL of CH₂Cl₂. The combined organic layers are dried over Na₂SO₄ and evaporated. The remaining oil is chromatographed in 50% CH₂Cl₂/hexane on 150 g of silica gel to give 1.7 g of 4-cyclopentyl-2-nitroaniline as an orange solid. ¹H NMR (400 MHz, CDCl₃) δ ppm 7.99 (d, 1H), 7.23-7.34 (m, 1H), 6.77 (d, 1H), 5.93 (br s, 2H), 2.87-3.00 (m, 1H), 1.94-2.14 (m, 2H), 1.71 (m, 2H), 1.65-1.87 (m, 2H), 1.54 (m, 2 H).

### Intermediate V

### 1-(3-Bromo-2-ethoxypropyl)-4-methylbenzene

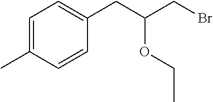

### Step 1 Preparation of ethyl-2-ethoxy-3-(4-methylphenyl)acrylate

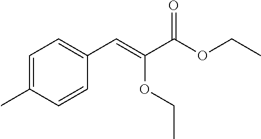 [SMILES: CCOC(=O)/C(=C/c1ccc(C)cc1)OCC]

To a cooled (0-5° C.) solution of ethoxyacetic acid, ethyl ester (1.50 mL, 11.01 mmol) and p-tolualdehyde (0.87 mL, 7.34 mmol) in tetrahydrofuran (20 mL) is added potassium tert-butoxide (988 mg, 8.81 mmol) portionwise. After 1 h, the ice bath is removed and the solution is stirred at ambient temperature overnight. The mixture is diluted with ethyl acetate and washed with saturated, aqueous NH₄C1, 0.1 N HCl, saturated, aqueous NaHCO₃, brine, dried with anhydrous sodium sulfate, filtered and concentrated. The residue is purifed by flash chromatography (hexane/dichloromethane 20-50% as eluent) to afford 905 mg of the title compound as an oil. ¹H NMR (CDCl₃) δ 1.39 (overlapping m, 6 H), 2.38 (s, 3H), 4.01 (q, 2H), 4.32 (q, 2H), 7.00 (s, 1H), 7.20 (d, 2H), 7.71 (d, 2H); MS (ESI−) for C₁₄H₁₈O₃ m/z 233.1 (M−H)⁻.

### Step 2 Preparation of ethyl 2-ethoxy-3-(4-methylphenyl)propanoate

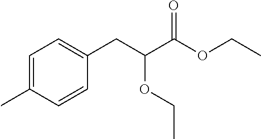 [SMILES: CCOC(=O)C(Cc1ccc(C)cc1)OCC]

To a flask containing ethyl 2-ethoxy-3-(4-methylphenyl)acrylate (905 mg, 3.86 mmol) and 10% Pd/C (90 mg) is added ethyl acetate (25 mL). The mixture is stirred under 1 atm of H₂ overnight, filtered through celite and concentrated to provide 868 mg of the title compound as an oil which is used without further purification. ¹H NMR (CDCl₃) δ 1.18 (t, 3H), 1.24 (t, 3H), 2.33 (s, 3H), 2.99 (d, 2H), 3.47 (m, 2H), 4.01 (t, 1H), 4.19 (q, 2 H), 7.13 (q, 4H); HPLC retention time: 4.97 min. (Method G).

### Step 3 Preparation of 2-ethoxy-3-(4-methylphenyl)propan-1-ol

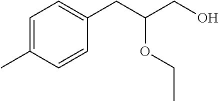

To a cooled (0-5° C.) flask containing lithium aluminum hydride (155 mg, 4.08 mmol) is slowly added tetrahydrofuran (10 mL). The mixture is stirred for 10 min at rt then cooled (0-5° C.). A solution of ethyl 2-ethoxy-3-(4-methylphenyl)propanoate (868 mg, 3.67 mmol) in THF (5 mL) is added dropwise and the mixture is stirred an additional 5 min. at 0-5° C. The ice bath is removed and the reaction mixture is stirred at rt overnight. The reaction is quenched with vigorous stirring by the sequential addition of water (0.15 mL), 15% aqueous NaOH (0.15 mL), and water (0.45 mL). The solids are removed by filtration through a pad of Celite and the filtrate dried (anhydrous sodium sulfate), filtered and concentrated at reduced pressure to provide 640 mg of the title compound as an oil which is used without further purification. ¹H NMR (CDCl₃) δ 1.21 (t, 3H), 1.97 (m, 1 H), 2.34 (s, 3H), 2.79 (m, 2H), 3.51 (overlapping m, 5H), 7.11 (s, 4H); HPLC retention time: 5.17 min. (Method G).

### Step 4 Preparation of 1-(3-bromo-2-ethoxypropyl)-4-methylbenzene

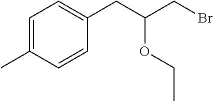

To a cooled (0-5° C.) solution of triphenylphosphine (1.296 g, 4.94 mmol) in DCM (10 mL) is added bromine (0.254 mL, 4.94 mmol) dropwise. After 10 min, additional triphenylphosphine (130 mg, 0.49 mmol) is added leading to the formation of a colorless solution. A solution of 2-ethoxy-3-(4-methylphenyl)propan-1-ol (640 mg, 3.29 mmol) in DCM (3 mL) is then added and the reaction is allowed to warm to rt and is stirred for 3 h. The reaction mixture is then transferred to a separatory funnel; washed with saturated, aqueous sodium bicarbonate, water and brine; and the combined organic layers are dried with anhydrous sodium sulfate and concentrated. The residue is purified by flash chromatography (hexane/ethyl acetate 1-5% as eluent) to give 733 mg of the title compound as an oil. ¹H NMR (CDCl₃) δ 1.97 (t, 3H), 2.34 (s, 3H), 2.91 (m, 2H), 3.43 (overlapping m, 5H), 7.14 (m, 4H); HPLC retention time: 5.49 min. (Method G).

### Intermediate W

### 3-(4-Chlorophenyl)-2-ethoxypropan-1-amine

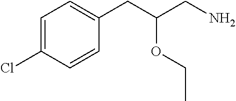

### Step 1 Preparation of ethyl 3-(4-chlorophenyl)-2-ethoxyacrylate

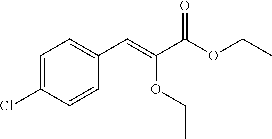 [SMILES: CCOC(=O)/C(=C/c1ccc(Cl)cc1)OCC]

To a cooled (0-5° C.) solution of ethoxyacetic acid, ethyl ester (0.750 mL, 5.50 mmol) and 4-chlorobenzaldehyde (516 mg, 3.67 mmol) in DMF (15 mL) is added potassium tert-butoxide (494 mg, 4.40 mmol) portionwise. After 1 h, the ice bath is removed and the solution is stirred at rt overnight. The reaction mixture is diluted with ethyl acetate and washed with saturated aqueous NH₄Cl, 0.1 N HCl, saturated NaHCO₃, brine, dried with anhydrous sodium sulfate, filtered and concentrated. The residue is purified by flash chromatography (hexane/dichloromethane 20-30% as eluent) to afford 510 mg of the title compound as an oil. ¹H NMR (CDCl₃) δ 1.38 (overlapping m, 6H), 4.03 (q, 2H), 4.32 (q, 2H), 6.93 (s, 1H), 7.35 (d, 2H), 7.74 (d, 2H); HPLC retention time: 5.55 min. (Method G).

### Step 2 Preparation of ethyl 3-(4-chlorophenyl)-2-ethoxypropanoate

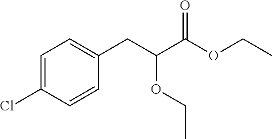 [SMILES: CCOC(=O)C(Cc1ccc(Cl)cc1)OCC]

To a flask containing ethyl 3-(4-chlorophenyl)-2-ethoxyacrylate (300 mg, 1.18 mmol), zinc dibromide (53 mg, 0.24 mmol) and 10% palladium on carbon (20 mg, 0.19 mmol) is added ethyl acetate (10 mL). The mixture is stirred under an atmosphere of H₂ overnight. The flask is evacuated, recharged with an atmosphere of H₂ and is stirred overnight. The mixture is filtered through celite and washed with water, brine, dried (sodium sulfate), filtered and concentrated to afford 300 mg of the title compound as an oil which is used without further purification. ¹H NMR (CDCl₃) δ 1.20 (t, 3H), 1.27 (t, 3H), 3.02 (m, 2H), 3.51 (m, 1H), 3.70 (m, 1H), 4.04 (t, 1H), 4.23 (q, 2H), 7.21 (d, 2H), 7.28 (d, 2H); HPLC retention time: 5.05 min. (Method G).

### Step 3 Preparation of 3-(4-chlorophenyl)-2-ethoxypropan-1-ol

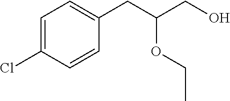

To a cooled (0-5° C.) flask containing lithium aluminum hydride (49 mg, 1.29 mmol) is slowly added tetrahydrofuran (4 mL). The mixture is stirred for 10 min. at rt then cooled (0-5° C.). A solution of ethyl 3-(4-chlorophenyl)-2-ethoxypropanoate (300 mg, 1.17 mmol) in THF (5 mL) is added dropwise and the mixture is stirred an additional 5 min. at 0-5° C. The ice bath is removed and the reaction is stirred at rt overnight. With vigorous stirring the reaction is quenched by the sequential addition of water (0.05 mL), 15% aqueous NaOH (0.05 mL), and water (0.15 mL). The solids are removed by filtration through a pad of Celite and the filtrate is dried (anhydrous sodium sulfate), filtered and concentrated at reduced pressure to provide 243 mg of the title compound as a pale yellow oil which is used without further purification. ¹H NMR (CDCl₃) δ 1.19 (t, 3H), 1.96 (br t, 1H), 2.80 (m, 2H), 3.50 (overlapping m, 5H), 7.16 (d, 2H), 7.28 (d, 2H); HPLC retention time: 3.85 min. (Method G).

### Step 4 Preparation of 1-(3-azido-2-ethoxypropyl)-4-chlorobenzene

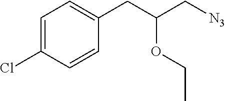 [SMILES: CCOC(CN=[N+]=[N-])Cc1ccc(Cl)cc1]

To a cooled (0-5° C.) solution of 3-(4-chlorophenyl)-2-ethoxypropan-1-ol (240 mg, 1.12 mmol) in DCM (8 mL) is added triethylamine (0.234 mL, 1.68 mmol) followed by methanesulfonyl chloride (0.108 mL, 1.40 mmol) dropwise. After 1 h, the ice bath is removed and the mixture is stirred at rt for 30 min. The reaction mixture is diluted with additional DCM and quenched by the addition of ice cold, saturated, aqueous NH₄Cl. The organic layer is separated and washed with cold water, cold 0.1 N HCl, cold saturated, aqueous NaHCO₃, dried (sodium sulfate), filtered and concentrated to afford an oil which is used without further purification.

To a solution of the crude mesylate prepared above in DMF (3 mL) is added sodium azide (581 mg, 8.94 mmol). The mixture is heated in a sealed tube at 65° C. overnight. The mixture is cooled to room temperature, diluted with ethyl acetate and washed with water, brine, dried (sodium sulfate), filtered and concentrated. The residue is purified by flash chromatography (hexane/ethyl acetate 10%) to afford 150 mg of the title compound as an oil. ¹H NMR (CDCl₃) δ 1.19 (t, 3H), 2.85 (m, 2H), 2.23 (m, 2H), 3.54 (overlapping m, 3 H), 7.16 (d, 2H), 7.28 (d, 2H); HPLC retention time: 5.30 min. (Method G).

### Step 4 Preparation of 3-(4-chlorophenyl)-2-ethoxypropan-1-amine

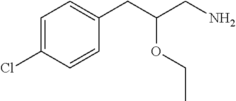

To a cold (0-5° C.), well-stirred solution of 1-(3-azido-2-ethoxypropyl)-4-chlorobenzene (620 mg, 2.59 mmol) in dry THF (13 mL) is added trimethylphosphine (3.88 mL of a 1.00 M solution in THF, 3.88 mmol). After 2 h, water (0.5 mL) is added, the ice bath is removed and stirring is continued for 2 h at rt. The reaction mixture is partitioned between brine and ethyl acetate, the layers separated and the aqueous layer extracted several times with ethyl acetate. The combined organic layers are dried (sodium sulfate), filtered and concentrated. The residue is purified by flash chromatography (DCM/MeOH 1-3% 0.07 NH₃) to afford 335 mg of the title compound as a solid. ¹H NMR (CDCl₃) δ 1.18 (t, 3H), 1.31 (br s, 2H), 2.75 (overlapping m, 4H), 3.45 (overlapping m, 3H), 7.15 (d, 2H), 7.27 (d, 2H); HPLC retention time: 3.02 min. (Method G).

### Intermediate X

### 1-[(1R)-3-Bromo-1-methylpropyl]-4-chlorobenzene

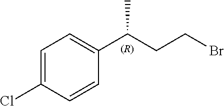

### Step 1 Preparation of triethyl (2S)-2-(4-chlorophenyl)propane-1,1,1-tricarboxylate

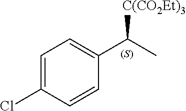

To a well-stirred solution of (1R)-1-(4-chlorophenyl)ethanol (1.00 g, 6.38 mmol) and triethylmethanetricarboxylate (2.69 mL, 12.8 mmol) in dry toluene (12.8 mL) under dry nitrogen is added a 1M solution of trimethylphosphine in THF (12.8 mL, 12.8 mmol) via syringe. The mixture is cooled at −78° C. and DIAD (2.51 mL, 12.8 mmol) is added slowly over a period of 15 min. The reaction is stirred at −78° C. for 1 h, the bath is removed and stirring is continued as the bath warmed to rt for an additional 4 h. The reaction mixture is concentrated, dissolved in diethyl ether (100 mL) and washed with 1N NaOH (2×50 mL) and 1N HCl (1×50 mL). The organics are dried with anhydrous sodium sulfate, concentrated and the residue is subjected to silica gel chromatography (230-400 mesh, 150 g, elution with 5 and 10% ethyl acetate/hexane) to give 1.90 g of triethyl (2S)-2-(4-chlorophenyl)propane-1,1,1-tricarboxylate as an oil. ¹H NMR (400 MHz, CDCl₃) δ ppm 1.20 (9H, t), 1.47 (3H, d), 3.82 (1H, q), 4.17 (6H, m), 7.24 (2H, d), 7.36 (2H, d); MS (ESI+) for C₁₈H₂₃ClO₆ m/z 371.1 (M+H)+; HPLC retention time: 5.20 min. (Method D).

### Step 2 Preparation of (3R)-3-(4-chlorophenyl)butanoic acid

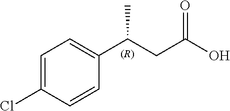

A solution of triethyl (2S)-2-(4-chlorophenyl)propane-1,1,1-tricarboxylate (1.90 g, 5.12 mmol) and 3.30 M sodium hydroxide (9.3 mL, 31 mmol) in MeOH (10 mL) is heated at 70° C. for 18 h. The reaction mixture is concentrated and re-dissolved in AcOH (30 mL). The mixture is heated at 120° C. for 18 h, cooled to rt, concentrated and azeotroped with toluene (3×) to remove any residual acetic acid. The reaction mixture is then partitioned between 10% citric acid and ethyl acetate (50 mL each), the layers are separated and the aqueous layer is extracted with ethyl acetate (3×30 mL). The combined organic layers are combined, dried with anhydrous sodium sulfate and concentrated to give 0.976 g of (3R)-3-(4-chlorophenyl)butanoic acid as an oil that solidified on standing. ¹H NMR (400 MHz, CDCl₃) δ ppm 1.30 (3H, d), 2.61 (2H, m), 3.26 (1H, m), 7.16 (2H, d), 7.27 (2H, d); MS (ESI−) for C₁₁H₁₁ClO₂ m/z 197.0 (M−H)−; HPLC retention time: 3.69 min. (Method D).

### Step 3 Preparation of (3R)-3-(4-chlorophenyl)butan-1-ol

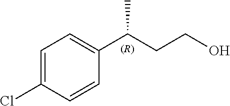

A slurry of LAH (0.669 g, 17.6 mmol) in dry THF (70 mL) is stirred at 0° C. under nitrogen and a solution of (3R)-3-(4-chlorophenyl)butanoic acid (1.75 g, 8.81 mmol) in THF (20 mL) added slowly. The reaction mixture is allowed to warm to rt and is stirred overnight. The reaction mixture is cooled at 0° C. and sodium sulfate decahydrate (200 mg) is added carefully. The mixture is then stirred at rt for 4 h. Water (2.0 mL) is added, the mixture is diluted with ether and is filtered through Celite. The salts are washed with ether and the filtrate is concentrated. The residue is subjected to silica gel chromatography (230-400 mesh, 350 g, elution with 20% ethyl acetate/hexane) to give 1.19 g of (3R)-3-(4-chlorophenyl)butan-1-ol as an oil that solidified on standing. ¹H NMR (400 MHz, CDCl₃) δ ppm 1.27 (3H, d), 1.83 (2H, m), 2.90 (1H, m), 3.56 (2H, m), 7.15 (2H, d), 7.26 (2H, d); HPLC retention time: 3.77 min. (Method D); optical rotation [α]_{D}²⁶ −37.5 (c=1.36, EtOH).

### Step 4 Preparation of 1-[(1R)-3-bromo-1-methylpropyl]-4-chlorobenzene

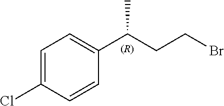

A solution of triphenylphosphine (1.6 g, 6.0 mmol) in DCM (40 mL, 600 mmol) is cooled at 0° C. and a solution of bromine (0.31 mL, 6.0 mmol) in DCM (10 mL) is added slowly over a period of 30 min. A solution of (3R)-3-(4-chlorophenyl)butan-1-ol (1.0 g, 5.4 mmol) in DCM (10 mL) is then added and the reaction mixture is allowed to warm to rt and is stirred for 24 h. The reaction mixture is then transferred to a separatory funnel; washed with saturated, aquous sodium bicarbonate, water and brine; the organics are dried with anhydrous sodium sulfate and concentrated. The residue is subjected to silica gel chromatography (230-400 mesh, 150 g, elution with 5% ethyl acetate/hexane) to give a total of 1.26 g of 1-[(1R)-3-bromo-1-methylpropyl]-4-chlorobenzene as a clear, colorless oil. ¹H NMR (400 MHz, CDCl₃) δ ppm 1.26 (3H, d), 2.08 (2H, m), 2.96 (1H, m), 3.15 (1 H, m), 3.32 (1H, m), 7.14 (2H, d), 7.28 (2H, d); HPLC retention time: 5.44 min.

### Intermediate Y

### 1-[(1S)-3-Bromo-1-methylpropyl]-4-chlorobenzene

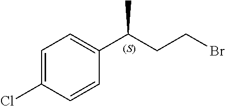

### Step 1 Preparation of triethyl (2R)-2-(4-chlorophenyl)propane-1,1,1-tricarboxylate

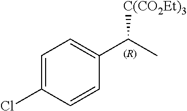

To a well-stirred solution of (1S)-1-(4-chlorophenyl)ethanol (2.00 g, 12.8 mmol) and triethyl methanetricarboxylate (5.37 mL, 25.5 mmol) in dry toluene (25 mL) under dry nitrogen is added a 1M solution of trimethylphosphine in THF (25.5 mL, 25.5 mmol) via syringe. The mixture is cooled at −78° C. and DIAD (5.03 mL, 25.5 mmol) is added slowly over a period of 15 min. The reaction mixture is stirred at −78° C. for 1 h, the bath is removed and stirring is continued as the bath warmed to rt for an additional 4 h. The reaction mixture is concentrated, dissolved in diethyl ether (200 mL) and washed with 1N NaOH (2×100 mL) and 1N HCl (1×100 mL). The organic layer is dried with anhydrous sodium sulfate, concentrated and the residue is subjected to silica gel chromatography (230-400 mesh, 350 g, elution with 5 and 10% ethyl acetate/hexane) to give 3.97 g of triethyl (2R)-2-(4-chlorophenyl)propane-1,1,1-tricarboxylate as an oil. ¹H NMR (400 MHz, CDCl₃) δ ppm 1.20 (9H, t), 1.47 (3H, d), 3.82 (1H, q), 4.17 (6H, m), 7.24 (2H, d), 7.36 (2H, d); MS (ESI⁺) for C₁₈H₂₃ClO₆ m/z 371.1 (M+H)⁺; HPLC retention time: 5.20 min. (Method D).

### Step 2 Preparation of (3S)-3-(4-chlorophenyl)butanoic acid

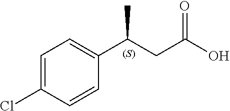

A solution of triethyl (2R)-2-(4-chlorophenyl)propane-1,1,1-tricarboxylate (3.97 g, 10.7 mmol) and 3.30 M sodium hydroxide (20 mL, 66 mmol) in MeOH (30 mL) is heated at 70° C. for 18 h. The reaction mixture is concentrated and re-dissolved in AcOH (60 mL). This mixture is heated at 120° C. for 18 h, cooled to rt, concentrated and azeotroped with toluene (3×) to remove any residual acetic acid. The reaction mixture is then partitioned between 10% citric acid and ethyl acetate (100 mL each), the layers are separated and the aqueous layer is extracted with ethyl acetate (3×630 mL). The organic layers are combined, dried with anhydrous sodium sulfate and concentrated to give 2.1 g of (3S)-3-(4-chlorophenyl)butanoic acid as an oil that solidifies on standing. ¹H NMR (400 MHz, CDCl₃) δ ppm 1.30 (3H, d), 2.61 (2H, m), 3.26 (1H, m), 7.16 (2H, d), 7.27 (2H, d); MS (ESI⁻) for C₁₁H₁₁ClO₂ m/z 197.0 (M−H)⁻; HPLC retention time: 3.69 min. (Method D).

### Step 3 Preparation of (3S)-3-(4-chlorophenyl)butan-1-ol

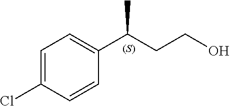

A slurry of LAH (0.80 g, 21.1 mmol) in dry THF (70 mL) is stirred at 0° C. under nitrogen and a solution of (3S)-3-(4-chlorophenyl)butanoic acid (2.10 g, 10.6 mmol) in THF (20 mL) added slowly. The reaction mixture is allowed to warm to rt and is stirred overnight. The reaction is cooled at 0° C. and sodium sulfate decahydrate (3 g) is added carefully. The mixture is then stirred at rt for 4 h. Water (2.0 mL) is added, the mixture is diluted with ether and filtered through Celite. The salts are washed with ether and the filtrate concentrated. The residue is subjected to silica gel chromatography (230-400 mesh, 350 g, elution with 20% ethyl acetate/hexane) to give 1.35 g of (35)-3-(4-chlorophenyl)butan-1-ol as an oil that solidified on standing. ¹H NMR (400 MHz, CDCl₃) δ ppm 1.27 (3H, d), 1.83 (2H, m), 2.90 (1H, m), 3.56 (2H, m), 7.15 (2H, d), 7.26 (2H, d); HPLC retention time: 3.77 min. (Method D); optical rotation [α]_{D}²⁶ +38.3 (C=1.31, EtOH).

### Step 4 Preparation of 1-[(1S)-3-bromo-1-methylpropyl]-4-chlorobenzene

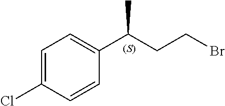

A solution of triphenylphosphine (1.6 g, 6.0 mmol) in DCM (40 mL, 600 mmol) is cooled at 0° C. and a solution of bromine (0.31 mL, 6.0 mmol) in DCM (10 mL) is added slowly over a period of 30 min. A solution of (3S)-3-(4-chlorophenyl)butan-1-ol (1.0 g, 5.4 mmol) in DCM (10 mL) is then added and the reaction is allowed to warm to rt and is stirred for 24 h. The reaction mixture is then transferred to a separatory funnel; washed with saturated, aqueous sodium bicarbonate, water and brine. The organic layer is dried with anhydrous sodium sulfate and concentrated. The residue is subjected to silica gel chromatography (230-400 mesh, 150 g, elution with 5% ethyl acetate/hexane) to give 1.15 g of 1-[(1S)-3-bromo-1-methylpropyl]-4-chlorobenzene as a clear colorless oil. ¹H NMR (400 MHz, CDCl₃) δ ppm 1.26 (3H, d), 2.08 (2H, m), 2.96 (1H, m), 3.15 (1H, m), 3.32 (1H, m), 7.14 (2H, d), 7.28 (2H, d); HPLC retention time: 5.44 min.

### Example 1

### 7,8-Dimethyl-10-(3-phenylpropvl)benzo[g]pteridine-2,4(3H,10H)-dione

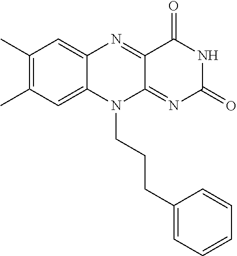 [SMILES: Cc1cc2nc3c(=O)[nH]c(=O)nc-3n(CCCc3ccccc3)c2cc1C]

### Step 1 Preparation of 4,5-dimethyl-2-nitro-N-(3-phenylpropyl)aniline

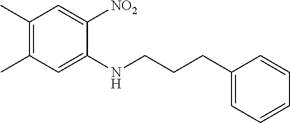 [SMILES: Cc1cc(NCCCc2ccccc2)c([N+](=O)[O-])cc1C]

To a 0° C. solution of 4,5-dimethyl-2-nitroaniline (3.0 g, 0.018 mol) in dry DMF (80 mL) is added sodium hydride (722 mg, 0.0180 mol) portion wise (gas evolution). After 15 min., the cooling bath is removed and solution is stirred 30 min. at rt. To this solution is added 1-bromo-3-phenylpropane (3.29 mL, 0.0217 mol) dropwise via syringe. After 18 h at rt, the reaction is concentrated in vacuo to remove DMF and the residue partitioned between DCM and saturated ammonium chloride solution (200 mL each). The layers are separated, the aqueous layer is extracted with DCM (3×100 mL), and the organics are combined, dried with anhydrous sodium sulfate and concentrated. The residue is subjected to silica gel chromatography (230-400 mesh, 350 g, elution with 5% EtOAc/hexane) to give 3.51 g (68%) of the desired product as an orange oil. ¹H NMR (400 MHz, CDCl₃) δ 2.06 (2H, m), 2.18 (3H, s), 2.24 (3H, s), 2.78 (2H, t), 3.30 (2H, t), 6.54 (1H, s), 7.23 (3H, m), 7.31 (2H, m), 7.93 (1H, s), 8.04 (1H, br s); MS (ESI⁺) for C₁₇H₂₀N₂O₂ m/z 285.2 (M+H)⁺, HPLC retention time: 5.54 min. (Method A).

### Step 2 Preparation of 4,5-dimethyl-N-(3-phenylpropyl)benzene-1,2-diamine

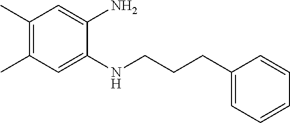 [SMILES: Cc1cc(N)c(NCCCc2ccccc2)cc1C]

A slurry of 4,5-dimethyl-2-nitro-N-(3-phenylpropyl)aniline (2.31 g, 8.12 mmol) and Raney Nickel (200 mg, 3 mmol) in ethanol (50 mL) is stirred at rt under 1 atm of hydrogen gas (balloon) for 18 h. The reaction mixture is diluted with ethyl acetate (50 mL), filtered through Celite and concentrated to give 2.0 g (96%) of the desired product as a white solid. ¹H NMR (400 MHz, DMSO-d₆) δ 1.86 (2H, m), 1.98 (3H, s), 2.01 (3H, s), 2.69 (2H, t), 2.97 (2H, t), 6.15 (1H, s), 6.33 (1H, s), 7.23 (5H, m); MS (ESI⁺) for C₁₇H₂₂N₂ m/z 255.3 (M+H)⁺, HPLC retention time: 3.36 min. (Method A).

### Step 3 Preparation of 7,8-dimethyl-10-(3-phenylpropyl)benzo[g]pteridine-2,4(3H,10H)-dione

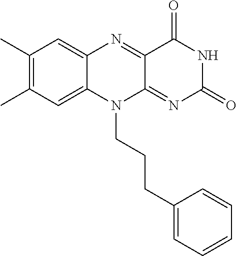 [SMILES: Cc1cc2nc3c(=O)[nH]c(=O)nc-3n(CCCc3ccccc3)c2cc1C]

To a mixture of 4,5-dimethyl-N-(3-phenylpropyl)benzene-1,2-diamine (1.00 g, 3.93 mmol), alloxan (0.63 g, 3.9 mmol) and boric acid (0.73 g, 12 mmol) is added acetic acid (10 mL). The reaction is then stirred at rt for 18 h. The acetic acid is removed in vacuo and the reaction is partitioned between DCM and brine (200 mL each). The layers are separated and the aqueous layer is extracted with DCM (5×50 mL). The organics are combined, dried with anhydrous sodium sulfate and concentrated. The residue is subjected to silica gel chromatography (adsorbed onto 40 g of silica gel; column, 230-400 mesh, 150 g, elution with 1 and 1.5% MeOH/CHCl₃) to give 645 mg (45%) of the desired product as an amorphous yellow solid. ¹H NMR (400 MHz, DMSO-d₆) δ 2.03 (2H, m), 2.38 (3H, s), 2.44 (3H, s), 2.80 (2H, t), 4.59 (2H, m), 7.26 (5H, m), 7.54 (1H, s), 7.89 (1H, s), 11.30 (1H, s); MS (ESI⁺) for C₂₁H₂₀N₄O₂ m/z 361.2 (M+H)⁺, HPLC retention time: 3.51 min. (Method A).

### Example 2

### 12-(3-Phenylpropyl)-8,9-dihydrol[1,4]benzodioxino[6,7-g]pteridine-2,4(3H,12H)-dione

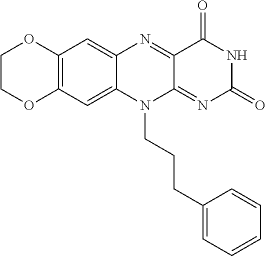 [SMILES: O=c1nc2n(CCCc3ccccc3)c3cc4c(cc3nc-2c(=O)[nH]1)OCCO4]

### Step 1 Preparation of 7-nitro-N-(3-phenylpropyl)-2,3-dihydro-1,4-benzodioxin-6-amine

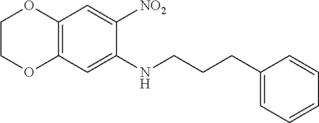 [SMILES: O=[N+]([O-])c1cc2c(cc1NCCCc1ccccc1)OCCO2]

To a 0° C. solution of 7-nitro-2,3-dihydro-1,4-benzodioxin-6-amine (0.500 g, 2.55 mmol) in dry DMF (12 mL, 150 mmol) is added sodium hydride (0.102 g, 2.55 mmol) portion wise (gas evolution). After 15 min, cooling bath is removed and solution stirred 30 min at rt. To this solution is added 1-bromo-3-phenylpropane (0.465 mL, 3.06 mmol) dropwise via syringe. After 18 h at rt, the reaction is concentrated in vacuo to remove DMF and the residue is partitioned between DCM and saturated ammonium chloride (50 mL each). The layers are separated, the aqueous is extracted with DCM (3×200 mL), and the organics are combined, dried with anhydrous sodium sulfate and concentrated. The residue is subjected to silica gel chromatography (230-400 mesh, 150 g, elution with 5, 10 and 20% EtOAc/hexane) to give 0.695 g (87%) of the desired product as an orange oil. ¹H NMR (400 MHz, CDCl₃) δ 2.05 (2H, m), 2.77 (2H, t), 3.22 (2H, m), 4.22 (2H, m), 4.33 (2H, m), 6.19 (1H, s), 7.22 (3H, m), 7.31 (2H, m), 7.74 (1H, s), 8.02 (1H, br s); MS (ESI⁺) for C₁₇H₁₈N₂O₄ m/z 315.1 (M+H)⁺, HPLC retention time: 4.92 min. (Method A).

### Step 2 Preparation of N-(3-phenylpropyl)-2,3-dihydro-1,4-benzodioxine-6,7-diamine (2B)

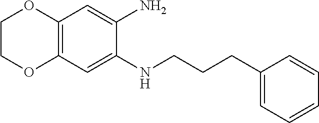 [SMILES: Nc1cc2c(cc1NCCCc1ccccc1)OCCO2]

A mixture of 7-nitro-N-(3-phenylpropyl)-2,3-dihydro-1,4-benzodioxin-6-amine (675 mg, 2.15 mmol) and Raney nickel (50 mg, 0.8 mmol) in ethanol (15 mL, 260 mmo) is stirred at rt under 1 atm of hydrogen gas (balloon) for 24 h. The mixture is filtered through Celite, the filter pad is washed with ethyl acetate and the filtrate is concentrated to give 590 mg (96%) of the desired product as an oil. MS (ESI⁺) for C₁₇H₂₀N₂O₂ m/z 285.3 (M+H)⁺, HPLC retention time: 3.07 min. (Method A).

### Step 3 Preparation of 12-(3-phenylpropyl)-8,9-dihydro[1,4]benzodioxino[6,7-g]pteridine-2,4(3H,12H)-dione

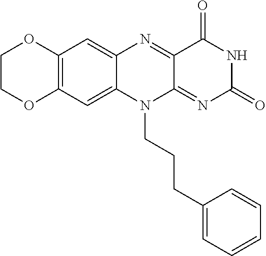 [SMILES: O=c1nc2n(CCCc3ccccc3)c3cc4c(cc3nc-2c(=O)[nH]1)OCCO4]

To a mixture of N-(3-phenylpropyl)-2,3-dihydro-1,4-benzodioxine-6,7-diamine (0.590 g, 2.07 mmol), boric acid (0.385 g, 6.22 mmol) and alloxan (0.349 g, 2.18 mmol) is added acetic acid (10 mL). The reaction is then stirred at rt for 18 h. The acetic acid is removed in vacuo, and the reaction is partitioned between DCM and brine (100 mL each), the layers are separated and the aqueous layer is extracted with DCM (5×30 mL). The organics are combined, dried with anhydrous sodium sulfate and concentrated. The residue is subjected to silica gel chromatography (adsorbed onto 40 g of silica gel; column, 230-400 mesh, 50 g, elution with 1 and 2% MeOH/CHCl₃) to give 169 mg (20%) of the desired product as an amorphous orange solid. ¹H NMR (400 MHz, DMSO-d₆) δ 2.00 (2H, t), 2.78 (2H, m), 4.40 (2H, m), 4.50 (2H, m), 4.57 (2H, d), 7.18 (1H, m), 7.26 (4H, m), 7.38 (1H, s), 7.59 (1H, s), 11.24 (1H, s); MS (ESI) for C₂₁H₁₈₀N₄O₄ m/z 391.1 (M+H)⁺, HPLC retention time: 3.08 min. (Method A).

### Example 3

### 7-Methyl-10-(2-phenoxyethyl)benzo[g]pteridine-2,4(3H,10H)-dione

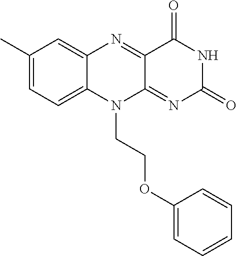 [SMILES: Cc1ccc2c(c1)nc1c(=O)[nH]c(=O)nc-1n2CCOc1ccccc1]

### Step 1 Preparation of 4-methyl-2-nitro-N-(2-phenoxyethyl)aniline

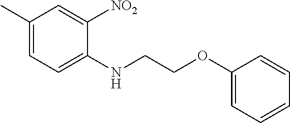 [SMILES: Cc1ccc(NCCOc2ccccc2)c([N+](=O)[O-])c1]

To a 0° C. solution of 3-nitro-4-aminotoluene (1.02 g, 6.70 mmol) in dry DMF (20 mL) is added sodium hydride (0.295 g, 7.37 mmol) portion wise (gas evolution). After 15 min., the cooling bath is removed and solution is stirred 30 min. at rt. To this solution is added (2-bromoethoxy)benzene (1.62 g, 8.04 mmol) dropwise via syringe. After 18 h at rt, the reaction is concentrated in vacuo to remove DMF and the residue is partitioned between DCM and saturated, aqueous ammonium chloride (50 mL each). The layers are separated, the aqueous is extracted with DCM (3×20 mL), and the organics are combined, dried with anhydrous sodium sulfate and concentrated. The residue is subjected to silica gel chromatography (230-400 mesh, 150 g, elution with 5, 10 and 20% EtOAc/hexane) to give 1.22 g (66%) of the desired product as an orange oil. ¹H NMR (400 MHz, CDCl₃) δ 2.28 (3H, s), 3.73 (2H, q), 4.24 (2H, t), 6.86 (1H, d), 6.94 (2H, d), 6.99 (1H, t), 7.31 (3H, t), 8.00 (1H, s), 8.23 (1H, br s); MS (ESI⁺) for C₁₅H₁₆N₂O₃ m/z 273.1 (M+H)⁺, HPLC retention time: 4.94 min. (Method A).

### Step 2 Preparation of 4-methyl-N1-(2-phenoxyethyl)benzene-1,2-diamine

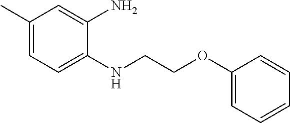 [SMILES: Cc1ccc(NCCOc2ccccc2)c(N)c1]

A slurry of 4-methyl-2-nitro-N-(2-phenoxyethyl)aniline (0.260 g, 0.955 mmol) and Raney nickel (30 mg, 0.5 mmol) in ethanol (10 mL) is stirred at rt under 1 atm of hydrogen gas for 18 h. The reaction is filtered through Celite, the filer pad is washed with ethyl acetate, and the filtrate is concentrated to give 227 mg (98%) of the desired product as an oil. MS (ESI⁺) for C₁₅H₁₈N₂O m/z 243.3 (M+H)⁺, HPLC retention time: 3.11 min. (Method A).

### Step 3 Preparation of 7-methyl-10-(2-phenoxyethyl)benzo[g]pteridine-2,4(3H,10H)-dione

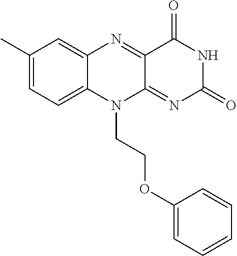 [SMILES: Cc1ccc2c(c1)nc1c(=O)[nH]c(=O)nc-1n2CCOc1ccccc1]

To a mixture of 4-methyl-N1-(2-phenoxyethyl)benzene-1,2-diamine (110.0 mg, 0.4540 mmol), alloxan (76.3 mg, 0.477 mmol) and boric acid (84.2 mg, 1.36 mmol) is added acetic acid (4.0 mL). The reaction is then stirred at rt for 48 h. The acetic acid is removed in vacuo, the reaction is partitioned between DCM and saturated sodium bicarbonate solution (50 mL each), the layers are separated and the aqueous layer is extracted with DCM (5×20 mL). The organics are combined, washed with 10% citric acid, dried with anhydrous sodium sulfate and concentrated. The residue is subjected to silica gel chromatography (adsorbed onto 40 g of silica gel; column; 230-400 mesh, 40 g, elution with 2 to 3% EtOH/CHCl₃) to give 96 mg of the impure product as an amorphous orange-yellow solid. The product is re-crystallized from ethanol to give 24 mg (15%) of the purified product as a yellow crystalline solid. ¹H NMR (400 MHz, DMSO-d₆) δ 4.39 (2 H, t), 5.00 (2H, t), 6.85 (2H, d), 6.90 (1H, t), 7.24 (2H, t), 7.80 (1H, dd), 7.93 (1H, s), 8.08 (1H, d), 11.38 (1H, s); MS (ESI⁺) for C₁₉H₁₆₀N₄O₃ m/z 349.1 (M+H)⁺, HPLC retention time: 3.19 min. (Method A).

### Example 4

### 10-[3-(2,6-Difluorophenybpropyl]-7,8-dimethylbenzo[g]pteridine-2,4(3H,10H)-dione

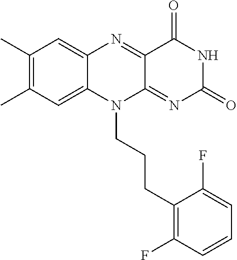 [SMILES: Cc1cc2nc3c(=O)[nH]c(=O)nc-3n(CCCc3c(F)cccc3F)c2cc1C]

### Step 1 Preparation of N-[3-(2,6-difluorophenyl)propyl]-4,5-dimethylbenzene-1,2-diamine

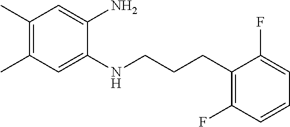 [SMILES: Cc1cc(N)c(NCCCc2c(F)cccc2F)cc1C]

A well-stirred slurry of 1-bromo-3-(2,6-difluorophenyl)propane (250 mg, 1.1 mmol), 4,5-dimethyl-o-phenylenediamine (0.58 g, 4.2 mmol), sodium bicarbonate (0.18 g, 2.1 mmol) and tetra-n-butylammonium iodide (0.039 g, 0.11 mmol) in toluene (10 mL) is heated at 70° C. under nitrogen for 18 h. The reaction is cooled to rt, partitioned between water and ethyl acetate (50 mL each), the layers are separated and the aqueous layer is extracted with ethyl acetate (3×20 mL). The organics are combined, dried with anhydrous sodium sulfate and concentrated. The residue is subjected to silica gel chromatography (230-400 mesh, 40 g, elution with 5% ethyl acetate/hexane) to give 160 mg (51%) of the desired product as an oil. ¹H NMR (400 MHz, CDCl₃) δ 1.95 (2H, m), 2.13 (3H, s), 2.17 (3H, s), 2.82 (2H, t), 3.13 (2H, t), 6.44 (1H, s), 6.53 (1H, s), 6.86 (2H, t), 7.15 (1H, m); MS (ESI⁺) for C₁₇H₂₀F₂N₂ m/z 291.1 (M+H)+, HPLC retention time: 3.48 min. (Method A).

### Step 2 Preparation of 10-[3-(2,6-difluorophenyl)propyl]-7,8-dimethylbenzo[g]pteridine-2,4(3H,10H)-dione

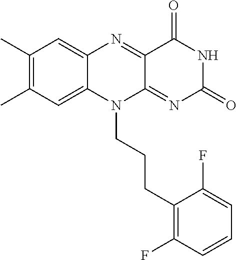 [SMILES: Cc1cc2nc3c(=O)[nH]c(=O)nc-3n(CCCc3c(F)cccc3F)c2cc1C]

To a mixture of N-[3-(2,6-difluorophenyl)propyl]-4,5-dimethylbenzene-1,2-diamine (158 mg, 0.54 mmol), alloxan (91.5 mg, 0.57 mmol) and boric acid (101 mg, 1.63 mmol) is added acetic acid (5 mL). The reaction is then stirred at rt for 72 h. The acetic acid is removed in vacuo. The reaction is slurried in water and filtered. The solid is triturated with hot ethanol, cooled and the precipitate is collected by filtration. The resulting solid provides 121 mg (56%) of the desired product as an amorphous orange solid. ¹H NMR (400 MHz, DMSO-d₆) δ 1.98 (2H, m), 2.32 (3H, s), 2.47 (3H, s), 2.86 (2H, t), 4.65 (2 H, t), 7.09 (2H, m), 7.33 (1H, m), 7.62 (1H, s), 7.90 (1H, s), 11.30 (1H, s); MS (ESI⁺) for C₂₁H₁₈F₂N₄O₂ m/z 397.1 (M+H)⁺, HPLC retention time: 3.57 min. (Method A).

### Example 5

### 10-(3-{4-[2-(Dimethylamino)ethoxy]phenyl}propyl)-7,8-dimethyl benzo[g]pteridine-2,4(3H,10H)-dione

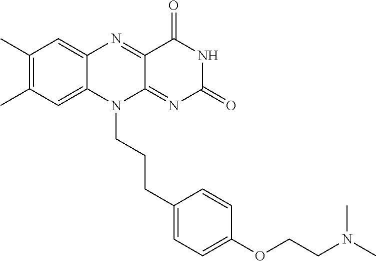

### Step 1 Preparation of methyl 3-{4-[2-(dimethylamino)ethoxy]phenyl}propanoate

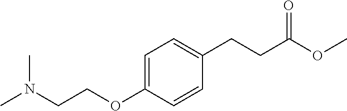

To a well-stirred solution of methyl 3-(4-hydroxyphenyl)propanoate (1.15 g, 6.38 mmol), N,N-dimethylaminoethanol (0.70 mL, 7.02 mmol) and triphenylphosphine (1.84 g, 7.02 mmol) in dry THF (50 mL) at rt is added DIAD (1.38 mL, 7.02 mmol) dropwise. The reaction mixture is stirred at rt for 18 h. The reaction is concentrated and subjected to silica gel chromatography (230-400 mesh, 150 g, elution with 20% THF/CHCl₃ followed by 2% MeOH (saturated with NH₃)/CHCl₃) to give 950 mg (59%) of the desired product as a clear colorless oil. ¹H NMR (400 MHz, CDCl₃) δ 2.30 (6H, s), 2.59 (2H, t), 2.71 (2 H, t), 2.88 (2H, t), 3.66 (3H, s), 4.03 (2H, t), 6.85 (2H, d), 7.10 (2H, d); MS (ESI⁺) for C₁₄H₂₁NO₃ m/z 252.2 (M+H)⁺, HPLC retention time: 2.41 min. (Method A).

### Step 2 Preparation of 3-{4-[2-(dimethylamino)ethoxy]phenyl}propan-1-ol

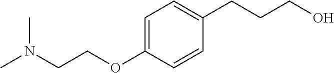 [SMILES: CN(C)CCOc1ccc(CCCO)cc1]

To a well-stirred solution of methyl 3-{4-[2-(dimethylamino)ethoxy]phenyl}propanoate (310 mg, 1.2 mmol) in dry THF (10 mL) at rt is added LiBH₄ (110 mg, 4.9 mmol). After 18 h at rt, the reaction is quenched with saturated ammonium chloride (10 mL), extracted with ethyl acetate (20 mL each) and the organics are combined, dried with anhydrous sodium sulfate and concentrated. The residue is subjected to silica gel chromatography (230-400 mesh, 50 g, elution with 1:1 ethyl acetate/hexane) to give 106 mg (38%) of the desired product as a clear colorless oil that solidified on standing. ¹H NMR (400 MHz, CDCl₃) δ 1.87 (2H, m), 2.67 (2H, m), 2.72 (6H, s), 3.20 (2H, t), 3.68 (2H, m), 4.37 (2 H, t), 6.84 (2H, m), 7.13 (2H, d); MS (ESI⁺) for C₁₃H₂₁NO₂ m/z 224.3 (M+H)⁺, HPLC retention time: 3.43 min. (Method A).

### Step 3 Preparation of N-(3-{4-[2-(dimethylamino)ethoxy]phenyl}propyl)-N-(4,5-dimethyl-2-nitrophenyl)-2,2,2-trifluoroacetamide

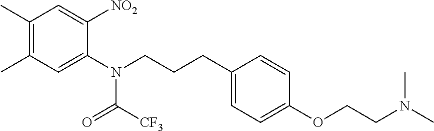

To a well stirred solution of 3-{4-[2-(dimethylamino)ethoxy]phenyl}propan-1-ol (106 mg, 0.47 mmol), N-(4,5-dimethyl-2-nitrophenyl)-2,2,2-trifluoroacetamide (0.124 g, 0.47 mmol) [Synthetic Commun. 1996 26, 4065] and triphenylphosphine (0.136 g, 0.52 mmol) in dry 1,2-dimethoxyethane (5.0 mL) at 0° C. is added DIAD (102 uL, 0.52 mmol) slowly via syringe. The reaction is allowed to stir at 0° C. for 30 min. and is warmed to rt overnight. The reaction is concentrated and the residue is subjected directly to silica gel chromatography (230-400 mesh, 50 g, elution with 2, 3, 4 and 5% MeOH (saturated with NH₃)/DCM) to give 120 mg (54%) of the product as an oil. MS (ESI⁺) for C₂₃H₂₈F₃N₃O₄ m/z 480.2 (M+Na)⁺, HPLC retention time: 4.60 min. (Method A).

### Step 4 Preparation of N-(3-{4-[2-(dimethylamino)ethoxy]phenyl}propyl)-4,5-dimethyl-2-nitroaniline

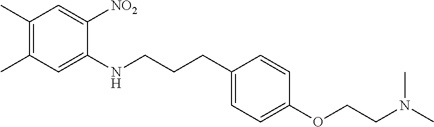

A solution of N-(3-{4-[2-(dimethylamino)ethoxy]phenyl}propyl)-N-(4,5-dimethyl-2-nitrophenyl)-2,2,2-trifluoroacetamide (120 mg, 0.26 mmol) and K₂CO₃ (0.106 g, 0.770 mmol) in MeOH (3.0 mL) is stirred at rt for 18 h. The reaction mixture is partitioned between 5% sodium carbonate and DCM (50 mL each), the layers are separated and the aqueous layer is extracted with DCM (3×20 mL). The organics are combined, dried with anhydrous sodium sulfate and concentrated. The residue is subjected to silica gel chromatography (230-400 mesh, 50 g, elution with 2 and 3% MeOH (saturated with NH₃)/DCM) to give 36 mg (38%) of the desired product as an oil. ¹H NMR (400 MHz, CDCl₃) δ 2.02 (2H, m), 2.17 (3H, s), 2.24 (3H, s), 2.70 (2H, m), 2.71 (6H, s), 3.20 (2H, t), 3.29 (2H, m), 4.38 (2H, t), 6.55 (1H, s), 6.84 (2H, d), 7.14 (2H, d), 7.92 (1H, s), 8.00 (1H, br s); MS (ESI⁺) for C₂₁H₂₉N₃O₃ m/z 372.2 (M+H)⁺, HPLC retention time: 5.38 min. (Method A).

### Step 5 Preparation of N-(3-{4-[2-(dimethylamino)ethoxy]phenyl}propyl)-4,5-dimethylbenzene-1,2-diamine

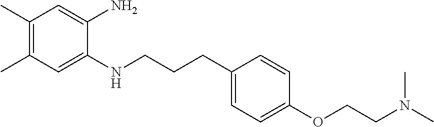

A slurry of N-(3-{4-[2-(dimethylamino)ethoxy]phenyl}propyl)-4,5-dimethyl-2-nitroaniline (36 mg, 0.097 mmol) and Raney nickel (20 mg, 0.3 mmol) in ethanol (5.0 mL, 86 mmol) is subjected to 1 atm of hydrogen gas for 18 h. The reaction mixture is filtered through Celite, the filter pad is washed with ethyl acetate and the organics are combined and concentrated to give 24 mg (72%) of desired product as a colorless oil. ¹H NMR (400 MHz, CDCl₃) δ 1.86 (2H, m), 2.04 (3H, s), 2.07 (3H, s), 2.27 (6H, s), 2.62 (2H, t), 2.69 (2H, t), 3.04 (2H, m), 4.00 (2H, t), 6.33 (1H, s), 6.44 (1H, s), 6.77 (2H, d), 7.04 (2 H, d); MS (ESI⁺) for C₂₁H₃₁N₃O m/z 342.3 (M+H)⁺, HPLC retention time: 2.54 min. (Method A).

### Step 6 Preparation of 10-(3-[4-[2-(dimethylamino)ethoxy]phenyl]propyl)-7,8-dimethylbenzo[g]pteridine-2,4(3H,10H)-dione

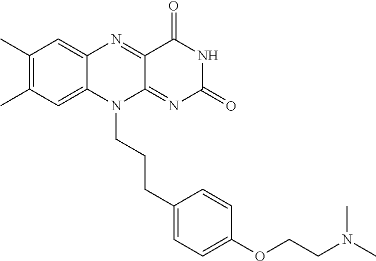

To a mixture of N-(3-{4-[2-(dimethylamino)ethoxy]phenyl}propyl)-4,5-dimethylbenzene-1,2-diamine (24.0 mg, 0.070 mmol), alloxan (11.2 mg, 0.070 mmol) and boric acid (13.0 mg, 0.211 mmol) is added acetic acid (2.0 mL). The reaction is then stirred at rt for 72 h. The acetic acid is removed in vacuo and the residue is subjected to silica gel chromatography (230-400 mesh, 50 g, elution with 5% MeOH/DCM then 5% MeOH (saturated with NH₃)/DCM) to give 21 mg (66%) of the desired product as an amorphous yellow solid. ¹H NMR (400 MHz, DMSO-d₆) δ 1.99 (2H, m), 2.20 (6H, s), 2.38 (3H, s), 2.44 (3H, s), 2.60 (2H, t), 2.72 (2H, t), 4.00 (2H, t), 4.57 (2H, m), 6.86 (2H, d), 7.17 (2 H, d), 7.49 (1H, s), 7.89 (1H, s), 11.30 (1H, s); MS (ESI⁺) for C₂₅H₂₉N₅O₃ m/z 448.2 (M+H)⁺, HPLC retention time: 2.54 min. (Method A).

### Example 6

### 10-[3-(4-Chlorophenyl)propyl]-7,8-dimethylbenzo[g]pteridine-2,4(3H,10H)-dione

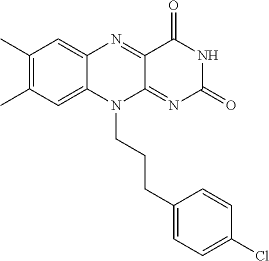 [SMILES: Cc1cc2nc3c(=O)[nH]c(=O)nc-3n(CCCc3ccc(Cl)cc3)c2cc1C]

### Step 1 Preparation of N-[3(4-chlorophenyl)propyl]-4,5-dimethyl-2-nitroaniline

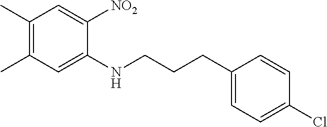 [SMILES: Cc1cc(NCCCc2ccc(Cl)cc2)c([N+](=O)[O-])cc1C]

A well-stirred slurry of 1-bromo-4,5-dimethyl-2-nitrobenzene (2.24 g, 9.72 mmol) [prepared by the method of Martin Langner, Chemistry—A European Journal, 2005, 11, 6254, the contents of which are incorporated by reference in their entirety], 3-(4-chlorophenyl)propan-1-amine (1.10 g, 6.48 mmol), cesium carbonate (4.22 g, 13.0 mmol) and (oxydi-2,1-phenylene)bis[diphenylphosphine] (1.05 g, 1.94 mmol) in toluene (10.4 mL, 97.2 mmol) is sparged with dry nitrogen for 5 min. Tris(dibenzylideneacetone)dipalladium(0) (0.594 g, 0.648 mmol) is added and sparging continued for an additional 5 min. The mixture is then heated in a 90° C. oil bath overnight. The mixture is cooled to rt, partitioned between 5% sodium carbonate and chloroform (50 mL each), the layers are separated and the aqueous layer is extracted with chloroform (3×20 mL). The organic layers are combined, dried with anhydrous sodium sulfate and is concentrated. The residue is subjected to silica gel chromatography (230-400 mesh, 50 g, elution with 30% CH₂Cl₂/hexane) to give 0.900 mg (74%) of desired product as a red solid. ¹H NMR (400 MHz, CDCl₃) δ 8.06 (br s, 1H), 7.95 (s, 1H), 7.26-7.31 (m, 2H), 7.16 (d, 2H), 6.55 (s, 1H), 3.30 (m, 2H), 2.77 (t, 2H), 2.26 (s, 3H), 2.20 (s, 3H), 2.06 (t, 2H); MS (ESI⁺) for C₁₇H₁₉ClN₂O₂ m/z 319 (M+H)⁺.

### Step 2 Preparation of N-[3-(4-chlorophenyl)propyl]-4,5-dimethylbenzene-1,2-diamine

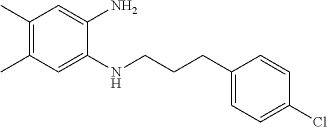 [SMILES: Cc1cc(N)c(NCCCc2ccc(Cl)cc2)cc1C]

N-[3-(4-chlorophenyl)propyl]-4,5-dimethyl-2-nitroaniline (2.90 g, 9.10 mmol) is added as a solution in 10 mL of EtOH to nickel (0.267 g, 4.55 mmol) and the mixture is stirred at rt under 1 atm of H₂. After 3 h, the nickel is removed by filtration through Celite and the filtrate is evaporated to provide 2.6 g (98%) of N-[3-(4-chlorophenyl)propyl]-4,5-dimethylbenzene-1,2-diamine that is used as is in the next step.

### Step 3 Preparation of 10-[3-(4-chlorophenyl)propyl]-7,8-dimethylbenzo[g]pteridine-2,4(3H,10H)-dione

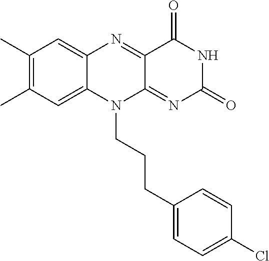 [SMILES: Cc1cc2nc3c(=O)[nH]c(=O)nc-3n(CCCc3ccc(Cl)cc3)c2cc1C]

To a mixture of N-[3-(4-chlorophenyl)propyl]-4,5-dimethylbenzene-1,2-diamine (2.90 g, 10.0 mmol), alloxan (1.69 g, 10.5 mmol) and boric acid (1.86 g, 30.1 mmol) is added 25 mL of HOAc. The mixture is then shaken at rt for 18 h. The mixture is concentrated on the rotovap to ½ of its volume and then diluted with 100 mL of water. The mixture is stirred at rt for 15 minutes and the solid is collected by filtration. Chromatography on silica gel (230-400 mesh) in 3% MeOH/CH₂Cl₂ gives 0.6 g (40%) of desired product as a red solid. ¹H NMR (400 MHz, DMSO-d₆) δ 11.31 (s, 1H), 7.89 (s, 1H), 7.58 (s, 1H), 7.33 (d, 2H), 7.29-7.37 (d, 2H), 4.60 (br s, 2H), 2.80 (t, 2H), 2.47 (s, 3H), 2.39 (s, 3H), 2.02 (d, 2 H); MS (ESI⁺) for C₂₁H₁₉ClN₄O₂ m/z 395 (M+H)⁺, HPLC retention time: 5.63 min. (Method B).

### Example 7

### Preparation of 10-[3-(4-hydroxyphenyl)propyl]-7,8-dimethylbenzo[g]pteridine-2,4(3H,10H)-dione

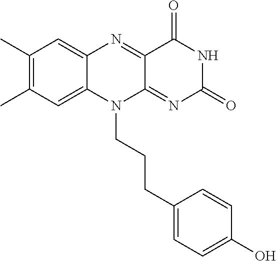 [SMILES: Cc1cc2nc3c(=O)[nH]c(=O)nc-3n(CCCc3ccc(O)cc3)c2cc1C]

To a 0° C. mixture of 10-[3-(4-methoxyphenyl)propyl]-7,8-dimethylbenzo[g]pteridine-2,4(3H,10H)-dione (0.039 g, 0.10 mmol) in DCM (10 mL) under nitrogen is added 1.00 M of boron tribromide in DCM (1.00 mL). After 2.5 h, ice (3 mL) is added followed by cold water (5 mL), and the mixture is stirred for 10 min. The reaction mixture is filtered and the solid is washed with water (4×2 mL), and then DCM (4×3 mL) to give an orange brown solid that is adsorbed onto silica gel and chromatographed on silica gel (230-400 mesh) using 6% MeOH/DCM giving the desired product as an orange solid (0.012 g, 32%); MS (ESI−) for C₂₁H₂₀N₄O₃ m/z 375.3 (M−H)⁻, HPLC retention time: 3.76 min. (Method B).

### Example 8

### Preparation of 10-[3-(3-hydroxyphenyl)propyl]-7,8-dimethylbenzo[g]pteridine-2,4(3H,10H)-dione

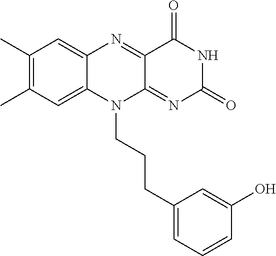 [SMILES: Cc1cc2nc3c(=O)[nH]c(=O)nc-3n(CCCc3cccc(O)c3)c2cc1C]

To a 0° C. mixture of 10-[3-(3-methoxyphenyl)propyl]-7,8-dimethylbenzo[g]pteridine-2,4(3H,10H)-dione (0.059 g, 0.15 mmol) in DCM (10 mL) under nitrogen is added 1.00 M of boron tribromide in DCM (1.5 mL, 1.5 mmol). After 2.5 h, ice (3 mL) is added followed by cold water (5 mL), and the mixture is stirred for 10 min. The reaction mixture is filtered and the solid is washed with water (4×2 mL) and then DCM (4×3 mL) to give product as an orange brown solid. The solid is dissolved in minimal DMSO and chromatographed (preparative reverse phase on C18 silica gel, Method L) using a gradient from 0% MeCN (1% TFA)/100% H₂O (1% TFA) to 55% MeCN (1% TFA)/45% H2O (1% TFA). The desired product is isolated as a yellow solid (0.022 g, 39%). MS (ESI⁺) for C₂₁H₂₀N₄O₃ m/z 377.09 (M+H)⁺, HPLC retention time: 3.85 min. (Method B).

### Example 9

### 8-Chloro-7-methyl-10-(3-phenylpropyl)benzo[g]pteridine-2,4(3H,10H)-dione

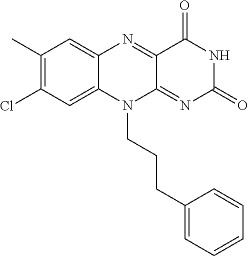 [SMILES: Cc1cc2nc3c(=O)[nH]c(=O)nc-3n(CCCc3ccccc3)c2cc1Cl]

### Step 1 Preparation of 5-chloro-4-methyl-2-nitro-N-(3-phenylpropyl)aniline

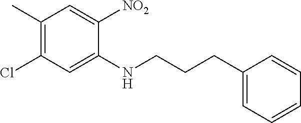 [SMILES: Cc1cc([N+](=O)[O-])c(NCCCc2ccccc2)cc1Cl]

To a 0-5° C. solution of 4-amino-2-chloro-5-nitrotoluene (2.50 g, 0.013 mol) in DMF (60 mL) is added sodium hydride (536 mg, 0.0134 mol) portion wise (gas evolution). After 15 min, the cooling bath is removed and the solution is stirred 30 min. at rt. To this solution is added 1-bromo-3-phenylpropane (2.44 mL, 0.016 mol) dropwise via syringe. After 18 h at rt, the reaction is concentrated in vacuo and the residue is partitioned between ethyl acetate and saturated aqueous ammonium chloride. The layers are separated and the organic layer is washed with brine, dried (anhydrous sodium sulfate), filtered and concentrated at reduced pressure. The residue is purified by flash chromatography (230-400 mesh, hexane/ethyl acetate (20%) as eluant) to afford 3.88 g (95%) of the desired product as an orange oil. ¹H NMR (400 MHz, CDCl₃) δ 2.07 (p, 2H), 2.29 (s, 3H), 2.79 (t, 2H), 3.27 (m, 2H), 6.81 (s, 1H), 7.27 (m, 5H), 7.94 (br t, 1H), 8.05 (s, 1H); MS (ESI⁺) for C₁₆H₁₇ClN₂O₂ m/z 305.1 (M+H)⁺, HPLC retention time: 7.88 min. (System A).

### Step 2 Preparation of 5-chloro-4-methyl-N-(3-phenylpropyl)benzene-1,2-diamine

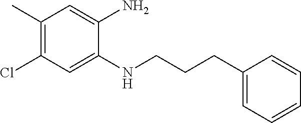 [SMILES: Cc1cc(N)c(NCCCc2ccccc2)cc1Cl]

A slurry of 5-chloro-4-methyl-2-nitro-N-(3-phenylpropyl)aniline (3.80 g, 12.5 mmol) and Raney nickel (400 mg, 6 mmol) in ethanol (70 mL) is stirred at rt under 1 atm of hydrogen gas (balloon) for 18 h. The reaction mixture is diluted with ethanol (70 mL), filtered through a pad of Celite and concentrated to give 3.09 g (90%) of desired product as a colorless solid that is used immediately in the next step. HPLC retention time: 5.63 min. (System A).

### Step 3 Preparation of 8-chloro-7-methyl-10-(3-phenylpropyl)benzo[g]pteridine-2,4(3H,10H)-dione

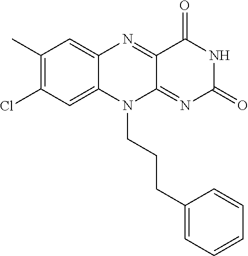 [SMILES: Cc1cc2nc3c(=O)[nH]c(=O)nc-3n(CCCc3ccccc3)c2cc1Cl]

To a mixture of 5-chloro-4-methyl-N-(3-phenylpropyl)benzene-1,2-diamine (3.09 g, 11.2 mmol), alloxan (1.80 g, 11.2 mmol) and boric acid (0.85 g, 22 mmol) is added acetic acid (40 mL). After 18 h at rt, volatiles are removed in vacuo and the residue is partitioned between ethyl acetate and saturated aqueous sodium bicarbonate. The layers are separated and the organic layer is washed with brine, dried (anhydrous sodium sulfate), filtered and concentrated at reduced pressure. The residue is purified by flash chromatography (230-400 mesh, CHCl₃/methanol (1-2%) as eluant) to afford 1.42 g (33%) of the desired product as an amorphous yellow solid. ¹H NMR (400 MHz, DMSO-d₆) δ 2.02 (p, 2H), 2.48 (s, 3H), 2.80 (t, 2H), 4.59 (br t, 2H), 7.23 (m, 5H), 7.95 (s, 1H), 8.13 (s, 1H), 11.42 (s, 1H); MS (ESI+) for C₂₀H₁₇ClN₄O₂ m/z 381.1 (M+H)⁺; HPLC retention time: 5.65 min. (System A).

### Example 10

### Preparation of 8-(cyclopentylamino)-7-methyl-10-(3-phenylpropyl)-benzo[g]pteridine-2,4(3H,10H)-dione

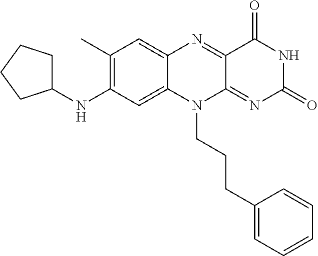 [SMILES: Cc1cc2nc3c(=O)[nH]c(=O)nc-3n(CCCc3ccccc3)c2cc1NC1CCCC1]

To a pressure tube containing a solution of 8-chloro-7-methyl-10-(3-phenylpropyl)benzo[g]pteridine-2,4(3H,10H)-dione (100 mg, 0.26 mmol) in DMF (3 mL) is added cyclopentanamine (0.259 mL, 2.62 mmol). The tube is sealed and the mixture is stirred for 16 h at 80° C. Concentration of the reaction mixture at reduced pressure provides a residue that is purified by flash chromatography (230-400 mesh, CH₂Cl₂/0.07 N methanolic ammonia (0-2%) as eluant) to afford 30 mg (26%) of the desired product as an amorphous red solid. ¹H NMR (400 MHz, DMSO-d₆) δ 1.67 (m, 6H), 1.99 (m, 4H), 2.27 (s, 3H), 2.81 (t, 2H), 3.92 (m, 1H), 4.58 (m, 2H), 6.28 (s, 1H), 6.64 (d, 1H), 7.25 (m, 5H), 7.64 (s, 1H), 10.95 (s, 1H); MS (ESI⁺) for C₂₅H₂₇N₅O₂ m/z 430.3 (M+H)⁺; HPLC retention time: 5.69 min. (System A).

### Example 11

### Preparation of 8-methoxy-7-methyl-10-(3-phenylpropyl)benzo[g]pteridine-2,4(3H,10H)-dione

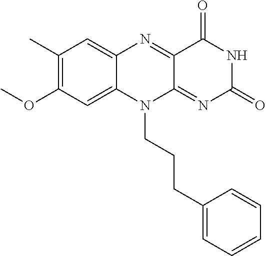 [SMILES: COc1cc2c(cc1C)nc1c(=O)[nH]c(=O)nc-1n2CCCc1ccccc1]

To a 0-5° C. cooled solution of 8-chloro-7-methyl-10-(3-phenylpropyl)benzo[g]pteridine-2,4(3H,10H)-dione (75 mg, 0.20 mmol) in methanol (3 mL) is added sodium methoxide (224 mg, 3.94 mmol). The ice bath is removed and the mixture is stirred for 16 h at 80° C. After cooling to rt, the reaction is quenched by the addition of a slight excess (based on sodium methoxide) of acetic acid. Concentration at reduced pressure provided a residue that is purified by flash chromatography (230-400 mesh, CHCl₃/methanol (0-2%) as eluant) to afford 57 mg (77%) of the desired product as an amorphous yellow solid. ¹H NMR (400 MHz, DMSO-d₆) δ 2.09 (p, 2H), 2.29 (s, 3H), 2.82 (t, 2H), 3.96 (s, 3H), 4.66 (br t, 2H), 6.93 (s, 1H), 7.24 (m, 5H), 7.91 (s, 1H), 11.25 (s, 1H); MS (ESI⁺) for C₂₁H₂₀N₄O₃ m/z 377.2 (M+H)⁺; HPLC retention time: 5.34 min. (System A).

### Example 12

### Preparation of 8-(cyclopropylamino)-7-methyl-10-(4-phenylbutyl)benzo[g]pteridine-2,4(3H,10H)-dione

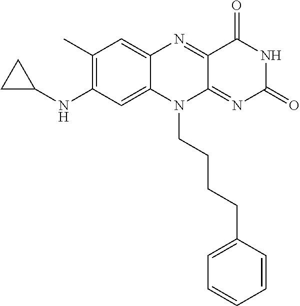 [SMILES: Cc1cc2nc3c(=O)[nH]c(=O)nc-3n(CCCCc3ccccc3)c2cc1NC1CC1]

To a pressure tube containing 8-chloro-7-methyl-10-(4-phenylbutyl)benzo[g]pteridine-2,4(3H,10H)-dione (prepared as described for 8-chloro-7-methyl-10-(3-phenylpropyl)benzo[g]pteridine-2,4(3H,10H)-dione from 4-amino-2-chloro-5-nitrotoluene and 1-bromo-4-phenylbutane) (75 mg, 0.19 mmol) is added cyclopropylamine (0.132 mL, 1.90 mmol) and NMP (2.7 mL). The tube is sealed and the mixture is stirred for 8 h at 80° C. Concentration in vacuo provides a residue that is purified by flash chromatography (230-400 mesh, CH₂Cl₂/0.07 N methanolic ammonia (0-2%) as eluant) to afford 60 mg (76%) of the desired product as an amorphous red solid. ¹H NMR (400 MHz, DMSO-d₆) δ 0.61 (m, 2H), 0.86 (m, 2H), 1.78 (m, 4H), 2.23 (s, 3H), 2.61 (m, 1H), 2.69 (br t, 2 H), 4.59 (m, 2H), 6.83 (s, 1H), 7.21 (m, 5H), 7.40 (s, 1H), 7.65 (s, 1H), 10.98 (s, 1H); MS (ESI⁺) for C₂₄H₂₅N₅O₂ m/z 416.2 (M+H)⁺; HPLC retention time: 5.39 min. (System A).

### Example 13

### Preparation of 7-methyl-10-(3-phenylpropyl)benzo[g]pteridine-2,4(3H, 10H)-dione

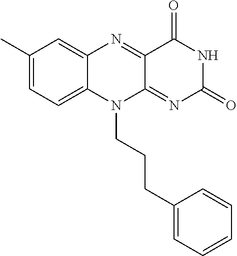 [SMILES: Cc1ccc2c(c1)nc1c(=O)[nH]c(=O)nc-1n2CCCc1ccccc1]

A mixture of 8-chloro-7-methyl-10-(3-phenylpropyl)benzo[g]pteridine-2,4(3H,10H)-dione (70 mg, 0.18 mmol), triethylamine (31 μL, 0.22 mmol) and palladium on carbon (10%, 10 mg) in isopropanol (70 mL) is stirred at rt under 1 atm of hydrogen gas (balloon) for 18 h. The reaction mixture is diluted with ethanol (70 mL), filtered through a pad of Celite and concentrated at reduced pressure. The residue is purified by flash chromatography (230-400 mesh, CHCl₃/methanol (0-2%) as eluant) to afford 19 mg (30%) of the desired product as an amorphous orange solid. ¹H NMR (400 MHz, DMSO-d₆) δ 2.03 (m, 2H), 2.80 (t, 2H), 4.63 (br t, 2H), 7.22 (m, 5H), 7.78 (m, 2H), 7.94 (s, 1H), 11.36 (s, 1H); MS (ESI+) for C₂₀H₁₈N₄O₂ m/z 347.2 (M+H)⁺; HPLC retention time: 5.17 min. (System A).

### Example 14

### Preparation of 10-(2-Amino-3-phenylpropyl)-7,8-dimethylbenzo[g]pteridine-2,4(3H,10H)-dione

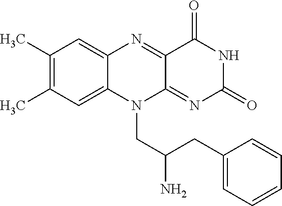 [SMILES: Cc1cc2nc3c(=O)[nH]c(=O)nc-3n(CC(N)Cc3ccccc3)c2cc1C]

### Step 1 Preparation of tert-butyl (1-((4,5-dimethyl-2-nitrophenyl)amino)-3-phenylpropan-2-yl)carbamate

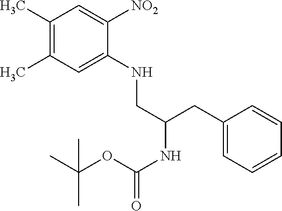 [SMILES: Cc1cc(NCC(Cc2ccccc2)NC(=O)OC(C)(C)C)c([N+](=O)[O-])cc1C]

tert-Butyl (1-((4,5-dimethyl-2-nitrophenyl)amino)-3-phenylpropan-2-yl)carbamate is prepared by heating a solution of 1-bromo-4,5-dimethyl-2-nitrobenzene (115 mg, 0.5 mmol) and tert-butyl (1-amino-3-phenylpropan-2-yl)carbamate (138 mg, 0.55 mmol) (commercially available from Accela Chembio Inc.) in DMSO (1 ml) at 130° C. for 1 h. The resulting mixture is diluted in DCM (40 ml), washed successively with H₂O (40 ml) and brine (40 ml), and then dried over Na₂SO₄, filtered and concentrated. The crude product is purified by column chromatography (mobile phase 0-40% EtOAc/Hex) to give desired product (123 mg, 62%) as a yellow powder. LC-MS m/z 399.9 [M+H], retention time 7.83 min.

### Step 2 Preparation of tert-butyl (1-((2-amino-4,5-dimethylphenyl)amino)-3-phenylpropan-2-yl)carbamate

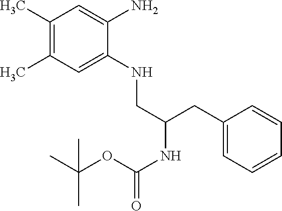 [SMILES: Cc1cc(N)c(NCC(Cc2ccccc2)NC(=O)OC(C)(C)C)cc1C]

tert-Butyl (1-((2-amino-4,5-dimethylphenyl)amino)-3-phenylpropan-2-yl)carbamate is prepared from tert-butyl (1-((4,5-dimethyl-2-nitrophenyl)amino)-3-phenylpropan-2-yl)carbamate (123 mg, 0.31 mmol) by catalytic reduction with Pd/C (10% Pd/C, 4% Pd w/w) and NaBH₄ (35 mg, 0.93 mmol) in a mixture of MeOH (5 ml) and EtOAc (5 ml) at room temperature under Ar. After 20 min, the reaction mixture is filtered through celite using MeOH (15 ml) and EtOAc (15 ml) to elute the product. The solvent is then evaporated to give desired product (quantitative) as a mixture of borate salts which is taken onto the next step without further purification.

### Step 3 Preparation of tert-butyl (1-(7,8-dimethyl-2,4-dioxo-3,4-dihydrobenzo[g]pteridin-10(2H)-yl)-3-phenylpropan-2-yl)carbamate

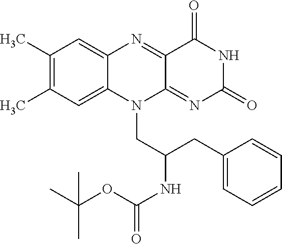 [SMILES: Cc1cc2nc3c(=O)[nH]c(=O)nc-3n(CC(Cc3ccccc3)NC(=O)OC(C)(C)C)c2cc1C]

tert-Butyl (1-(7,8-dimethyl-2,4-dioxo-3,4-dihydrobenzo[g]pteridin-10(2H)-yl)-3-phenylpropan-2-yl)carbamate is prepared by stirring the crude tert-butyl (1-((2-amino-4,5-dimethylphenyl)amino)-3-phenylpropan-2-yl)carbamate (0.31 mmol), alloxan monohydrate (53 mg, 0.33 mmol) and boric acid (38 mg, 0.62 mmol) in AcOH (10 ml) at rt for 1.5 h. The reaction mixture is then evaporated to dryness, and the crude product is purified by column chromatography (mobile phase 0-100% EtOAc in hexanes, then 0-15% MeOH in DCM). The desired product is isolated as a bright orange powder (127 mg, 86%). LC-MS m/z 476.1 [M+H], retention time 6.91 min.

### Step 4 Preparation of 10-(2-amino-3-phenylpropyl)-7,8-dimethylbenzo[g]pteridine-2,4(3H,10H)-dione

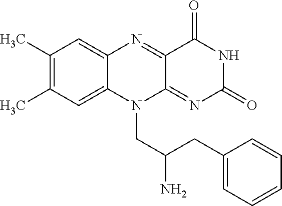 [SMILES: Cc1cc2nc3c(=O)[nH]c(=O)nc-3n(CC(N)Cc3ccccc3)c2cc1C]

tert-Butyl (1-(7,8-dimethyl-2,4-dioxo-3,4-dihydrobenzo[g]pteridin-10(2H)-yl)-3-phenylpropan-2-yl)carbamate (71 mg, 0.15 mmol) is dissolved in DCM (6 ml) at room temperature, and then TFA (1.5 ml) is added in one portion and the solution is stirred at room temperature for 45 min. The reaction mixture is evaporated and the resulting crude product is lyophilized. The mixture is dry-loaded onto silica gel, and then is purified by column chromatography (0 to 15% MeOH in DCM) to give 10-(2-amino-3-phenylpropyl)-7,8-dimethylbenzo[g]pteridine-2,4(3H,10H)-dione (47 mg, 83%) as a yellow powder. ¹H NMR (400 MHz, DMSO-d₆) δ 2.27 (s, 3H), 2.36 (s, 3H), 3.08 (s, 2H), 3.79 (s, 1H), 4.40 (d, 1H), 5.15 (m, 1H), 6.60 (s, 1H), 7.37 (s, 5H), 7.68 (br s, 2H), 7.91 (s, 1H), 11.45 (s, 1H). LC-MS m/z 376.1 [M+H], retention time 5.69 min.

### Example 15

### 8-Cyclopropyl-7-methyl-10-(3-phenylpropyl)benzo[g]pteridine-2,4(3H,10H)-dione

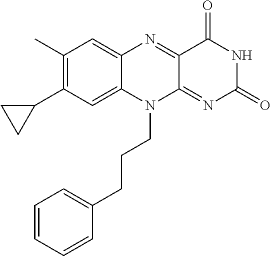 [SMILES: Cc1cc2nc3c(=O)[nH]c(=O)nc-3n(CCCc3ccccc3)c2cc1C1CC1]

### Step 1 Preparation of 4-amino-2-cyclopropyl-5-nitrotoluene

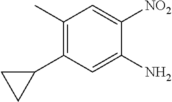

A well-stirred slurry of 4-amino-2-chloro-5-nitrotoluene (640 mg, 3.4 mmol), cyclopropylboronic acid (585 mg, 6.81 mmol) and cesium carbonate (3.3 g, 10.2 mmol) in anhydrous 1,4-dioxane (12 mL) is sparged with nitrogen for 10 min. [1,1′-Bis(diphenylphosphino)ferrocene]dichloropalladium(II) complex with dichloromethane (1:1) (560 mg, 0.68 mmol) is added and sparging is continued for another 10 min. The reaction is then heated at 90° C. for 24 h. The reaction is cooled, diluted with DCM (100 mL) and filtered through Celite. The organics are washed with saturated bicarbonate solution, brine, dried with anhydrous sodium sulfate and concentrated. The residue is chromatographed on silica gel (Silicycle, 230-400 mesh, 150 g, elution with 5 then 7.5% ethyl acetate/hexane) to give desired product (380 mg, 57%) as an orange solid. ¹H NMR (400 MHz, CDCl₃) δ ppm 0.68 (2H, m), 1.04 (2H, m), 1.88 (1H, m), 2.34 (3H, s), 5.92 (2H, br s), 6.33 (1H, s), 7.89 (1H, s); MS (ESI⁺) for C₁₀H₁₂N₂O₂ m/z 193.2 (M+H)⁺, retention time: 4.17 min. (Method D).

### Step 2 Preparation of 5-cyclopropyl-4-methyl-2-nitro-N-(3-phenylpropyl)aniline

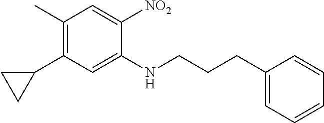 [SMILES: Cc1cc([N+](=O)[O-])c(NCCCc2ccccc2)cc1C1CC1]

To a 0° C. solution of 4-amino-2-cyclopropyl-5-nitrotoluene (0.775 g, 4.03 mmol) in dry DMF (20 mL) is added sodium hydride (161 mg, 4.03 mmol) portionwise. After 15 min, the cooling bath is removed and the solution is stirred at rt for 30 min. To this solution is added 1-bromo-3-phenylpropane (0.736 mL, 4.84 mmol) dropwise via syringe. After 18 h at rt, the reaction is concentrated in vacuo to remove DMF and the residue partitioned between DCM and saturated ammonium chloride (100 mL each). The layers are separated, the aqueous layer is extracted with DCM (3×40 mL), and the organics combined, dried with anhydrous sodium sulfate and concentrated. The residue is subjected to silica gel chromatography (Silicycle, 230-400 mesh,150 g, elution with 10% EtOAc/hexane) to give desired product (1.16 g, 92%) as an orange oil. ¹H NMR (400 MHz, CDCl₃) δ ppm 0.66 (2H, m), 1.04 (2H, m), 1.90 (1H, m), 2.06 (2H, m), 2.34 (3H, s), 2.80 (2H, t), 3.31 (2H, m), 6.32 (1H, s), 7.30 (5H, m), 7.96 (1H, s), 8.09 (1H, br s); MS (ESI⁺) for C₁₉H₂₂N₂O₂ m/z 311.1 (M+H)⁺, retention time: 5.68 min. (Method D).

### Step 3 Preparation of 5-cyclopropyl-4-methyl-N-(3-phenylpropyl)benzene-1,2-diamine

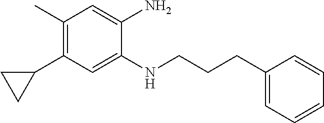 [SMILES: Cc1cc(N)c(NCCCc2ccccc2)cc1C1CC1]

A slurry of 5-cyclopropyl-4-methyl-2-nitro-N-(3-phenylpropyl)aniline (1.12 g, 3.61 mmol) and Raney nickel (0.0424 g, 0.722 mmol) in ethanol (25 mL) is stirred and exposed to 1 atm of hydrogen gas (balloon) for 18 h. The slurry is diluted with ethyl acetate (50 mL) and filtered through Celite. The filter pad is washed with ethyl acetate (2×25 mL) and the filtrate is concentrated to give desired product (980 mg, 96%) as an oil. ¹H NMR (400 MHz, CDCl₃) δ ppm 0.54 (2H, m), 0.84 (2H, m), 1.80 (1H, m), 1.99 (2H, m), 2.29 (3H, s), 2.77 (2H, t), 3.12 (2H, t), 6.33 (1H, s), 6.54 (1H, s), 7.29 (5H, m); MS (ESI⁺) for C₁₉H₂₄N₂ m/z 281.2 (M+H)⁺, retention time: 3.62 min. (Method D).

### Step 4 Preparation of 8-cyclopropyl-7-methyl-10-(3-phenylpropyl)-benzo[g]pteridine-2,4(3H,10H)-dione

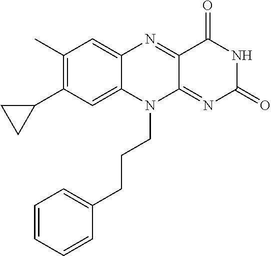 [SMILES: Cc1cc2nc3c(=O)[nH]c(=O)nc-3n(CCCc3ccccc3)c2cc1C1CC1]

A slurry of 5-cyclopropyl-4-methyl-N-(3-phenylpropyl)benzene-1,2-diamine (1.0 g, 3.6 mmol), alloxan (0.599 g, 3.74 mmol) and boric acid (0.662 g, 10.7 mmol) in acetic acid (20 mL) is stirred under nitrogen for 18 h. The reaction is concentrated, suspended in water and filtered. The solids are washed with water, diethyl ether and air dried. This solid was adsorbed onto silica gel (30 g) and subjected to silica gel chromatography (Silicycle, 230-400 mesh, 150 g, elution with 1, 2 and 3% EtOH/CHCl₃) to give 755 mg (54%) of the product as an orange solid. ¹H NMR (400 MHz, DMSO-d6) 0.77 (2H, m), 1.11 (2H, m), 2.00 (2H, m), 2.13 (1H, m), 2.53 (3H, s), 2.78 (2H, t), 4.60 (2H, m), 7.01 (1H, s), 7.26 (5H, m), 7.90 (1H, s), 11.30 (1H, s); MS (ESI+) for C₂₃H₂₂N₄O₂ m/z 387.1 (M+H)+, retention time: 3.79 min. (Method D).

### Example 16

### 7,8-Dimethyl-5-(3-phenylpropyl)pyrido[3,4-b]quinoxaline-1,3(2H,5H)-dione

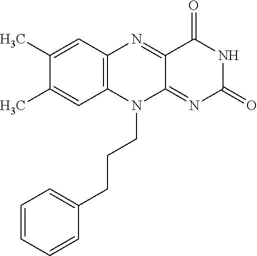 [SMILES: Cc1cc2nc3c(=O)[nH]c(=O)nc-3n(CCCc3ccccc3)c2cc1C]

Step 1 Preparation of ethyl (3Z)-3-(2-ethoxy-2-oxoethylidene)-6,7-dimethyl-4-(3-phenylpropyl)-3,4-dihydroquinoxaline-2-carboxylate

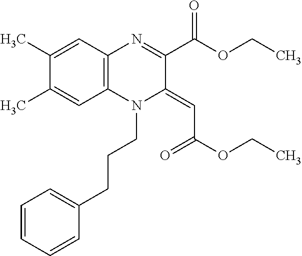 [SMILES: CCOC(=O)/C=C1/C(C(=O)OCC)=Nc2cc(C)c(C)cc2N1CCCc1ccccc1]

Cesium carbonate (5.38 g, 16.5 mmol) is added to a solution of 4,5-dimethyl-N-(3-phenylpropyl)benzene-1,2-diamine (0.600 g, 2.36 mmol) in 40 mL of 1:1 DMF/CH₂Cl₂ followed by diethyl 2-bromo-3-oxopentanedioate (4.64 g, 16.5 mmol) and the mixture is stirred at rt under N₂ overnight. The mixture is evaporated to dryness and the residue is partitioned between 50 mL of CH₂Cl₂ and 50 mL of water. The layers are separated and the aqueous phase is extracted with 2×50 mL of CH₂Cl₂. The combined organic layers are extracted with 3×50 mL of water. Drying over Na₂SO₄ and evaporation gives 3.5 g of a red oil. Chromatography on 150 g of silica gel in 30% EtOAc/hexane gives desired product (0.57 g, 56%) as a red solid. 1H NMR (400 MHz, CDCl₃) δ ppm 7.53 (s, 1H), 7.38 (d, 1H), 7.41 (d, 1H), 7.27-7.34 (m, 3H), 6.49 (s, 1H), 5.06 (s, 1H), 4.40 (q, 2H), 4.15 (q, 2H), 3.80 (q, 2H), 2.84 (q, 2H), 2.24 (m, 6H), 2.15 (m, 2H), 1.43 (t, 3H), 1.31 (t, 3H); MS (ESI⁺) for C₂₆H₃₀N₂O₄ m/z 435 (M+H)⁺.

### Step 2 Preparation of 7,8-dimethyl-5-(3-phenylpropyl)pyrido[3,4-b]ouinoxaline-1,3(2H,5H)-dione

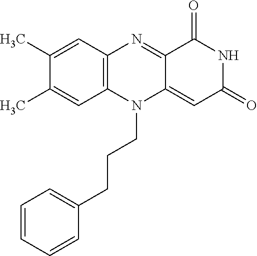 [SMILES: Cc1cc2nc3c(=O)[nH]c(=O)cc-3n(CCCc3ccccc3)c2cc1C]

Ethyl (3Z)-3-(2-ethoxy-2-oxoethyl idene)-6,7-dimethyl-4-(3-phenylpropyl)-3,4-dihydroquinoxaline-2-carboxylate (0.300 g, 0.690 mmol) is taken up in 20 mL of MeOH and the solution is cooled in an ice water bath. Ammonia gas is bubbled through the solution for 5 minutes in a pressure tube. The solution is stirred at rt overnight in the capped pressure tube. The pressure tube is opened slowly to allow NH₃ to evolve. The remaining solution is evaporated to give 0.2 g of a dark solid. The solid is adsorbed onto silica gel and chromatographed on 50 g of silica gel. The column is eluted with 1% MeOH/DCM (IL) followed by 1.5% MeOH/CH₂Cl₂ (1.5 L). The product elutes in the 1.5% MeOH/DCM. Evaporation of the fractions containing product gives 0.07 g of a purple solid. Crystallization from CH₃CN (25 mL) gives 12 mg of the product as a purple solid. ¹H NMR (400 MHz, DMSO-d₆) δ ppm 11.17 (s, 1H), 7.63 (s, 1H), 7.20-7.36 (m, 6H), 5.27 (d, 1H), 4.01 (br s, 2H), 2.82 (t, 2H), 2.35 (s, 3H), 2.29 (s, 3H), 1.93 (br s, 2H); MS (ESI⁺) for C₂₂H₂₁N₃O₂ m/z 360 (M+H)⁺, HPLC retention time: 3.84 min. (Method D).

### Example 17

### 10-(2-Isopropoxy-3-phenylpropv-1)-7,8-dimethylbenzo[g]pteridine-2,4(3H,10H)-dione

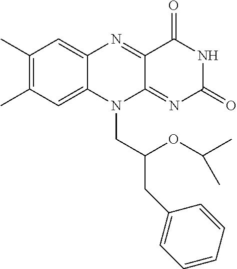 [SMILES: Cc1cc2nc3c(=O)[nH]c(=O)nc-3n(CC(Cc3ccccc3)OC(C)C)c2cc1C]

### Step 1 Preparation of N-(2-isopropoxy-3-phenylpropyl)-4,5-dimethyl-2-nitroaniline

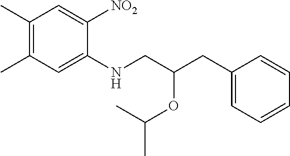 [SMILES: Cc1cc(NCC(Cc2ccccc2)OC(C)C)c([N+](=O)[O-])cc1C]

A well-stirred slurry of 1-bromo-4,5-dimethyl-2-nitrobenzene (0.295 g, 1.28 mmol), (3-amino-2-isopropoxypropyl)benzene (0.177 g, 0.916 mmol), Cs₂CO₃ (597 mg, 1.83 mmol) and (oxydi-2,1-phenylene)bis[diphenylphosphine] (74.0 mg, 0.137 mmol) in toluene (8 mL, 80 mmol) is sparged with dry nitrogen for 5 min. Tris(dibenzylideneacetone)-dipalladium(0) (41.9 mg, 0.0458 mmol) is added and sparging is continued for an additional 5 min. The reaction is then heated at 90° C. for 18 h. The reaction is cooled to rt, partitioned between water and DCM (50 mL each), the layers are separated and the aqueous layer is extracted with DCM (3×20 mL). The organics are combined, dried with anhydrous sodium sulfate and concentrated. The residue is subjected to silica gel chromatography (230-400 mesh, 150 g, elution with DCM) to give 91 mg (29%) of N-(2-isopropoxy-3-phenylpropyl)-4,5-dimethyl-2-nitroaniline as an orange oil. ¹H NMR (400 MHz, CDCl₃) δ 1.15 (3H, d), 1.19 (3H, d), 2.17 (3H, s), 2.19 (3H, s), 2.81 (1H, dd), 3.00 (1H, dd), 3.18 (1H, m), 3.36 (1H, m), 3.70 (1H, hep), 3.83 (1H, m), 6.40 (1H, s), 7.25-7.37 (5H, m), 7.93 (1H, s), 8.23 (1H, br t); MS (ESI⁺) for C₂₀H₂₆N₂O₃ m/z 343.4 (M+H)⁺; HPLC retention time: 5.54 min. (Method D).

### Step 2 Preparation of N-(2-isopropoxy-3-phenylpropyl)-4,5-dimethylbenzene-1,2-diamine

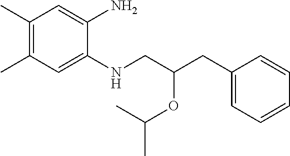 [SMILES: Cc1cc(N)c(NCC(Cc2ccccc2)OC(C)C)cc1C]

A slurry of N-(2-isopropoxy-3-phenylpropyl)-4,5-dimethyl-2-nitroaniline (91.0 mg, 0.266 mmol) and Raney Nickel (0.0156 g, 0.266 mmol) in EtOH (5 mL, 80 mmol) is subjected to 1 atm of hydrogen gas for 4 h. The mixture is diluted with ethyl acetate (20 mL), filtered through Celite and the filter pad is washed with additional ethyl acetate (20 mL). The filtrates are combined and concentrated to give 68 mg (82%) of N-(2-isopropoxy-3-phenylpropyl)-4,5-dimethylbenzene-1,2-diamine as an oil. MS (ESI⁺) for C₂₀H₂₈N₂O m/z 313.4 (M+H)⁺; HPLC retention time: 3.65 min. (Method D).

### Step 3 Preparation of 10-(2-isopropoxy-3-phenylpropyl)-7,8-dimethylbenzo[g]pteridine-2,4(3H,10H)-dione

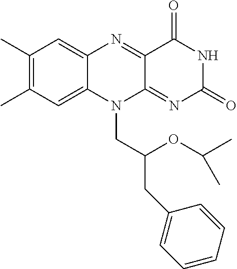 [SMILES: Cc1cc2nc3c(=O)[nH]c(=O)nc-3n(CC(Cc3ccccc3)OC(C)C)c2cc1C]

To a well-stirred solution of N-(2-isopropoxy-3-phenylpropyl)-4,5-dimethylbenzene-1,2-diamine (68 mg, 0.22 mmol) and alloxan (38.3 mg, 0.239 mmol) in acetic acid (3.0 mL) is added boric acid (40.4 mg, 0.653 mmol). The reaction mixture is then stirred at rt for 18 h, concentrated and azeotroped with toluene. The yellow solid is adsorbed onto silica gel (5 g) and subjected to silica gel chromatography (230-400 mesh, 50 g, elution with 1, 1.5 and 2% MeOH/DCM) to give 60 mg (66%) of 10-(2-isopropoxy-3-phenylpropyl)-7,8-dimethylbenzo[g]pteridine-2,4(3H,10H)-dione as an amorphous yellow solid. ¹H NMR (400 MHz, DMSO-d6) δ 0.42 (3H, m), 0.62 (3H, d), 2.39 (3H, s), 2.86 (2H, m), 3.08 (1 H, m), 4.12 (1H, m), 4.64 (2H, m), 7.24 (5H, m), 7.76 (1H, s), 7.88 (1H, s), 11.34 (1H, s); MS (ESI⁺) for C₂₄H₂₆N₄O₃ m/z 419.5 (M+H)⁺; HPLC retention time: 3.85 min. (Method D).

### Example 18

### 8-[(2,6-Dimethylmorpholin-4-yl)methyl]-7-methyl-10-(3-phenylpropyl)benzo[g]pteridine-2,4(3H,10H)-dione

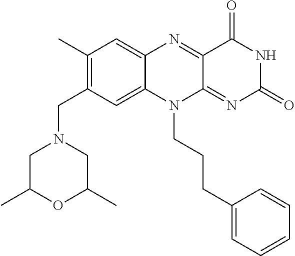 [SMILES: Cc1cc2nc3c(=O)[nH]c(=O)nc-3n(CCCc3ccccc3)c2cc1CN1CC(C)OC(C)C1]

### Step 1 Preparation of 8-(bromomethyl)-7-methyl-10-(3-phenylpropyl)benzo[g]pteridine-2,4(3H,10H)-dione

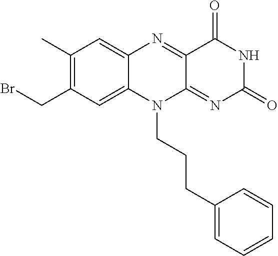 [SMILES: Cc1cc2nc3c(=O)[nH]c(=O)nc-3n(CCCc3ccccc3)c2cc1CBr]

To a well-stirred solution of 7,8-dimethyl-10-(3-phenylpropyl)benzo[g]pteridine-2,4(3H,10H)-dione (231.0 mg, 0.6409 mmol) in 1,4-dioxane (10 mL, 100 mmol) at rt under nitrogen is added benzoyl peroxide (77.63 mg, 0.3205 mmol) as a solid. The reaction is brought to reflux and after 15 min., a solution of Br₂ (72.64 uL, 1.410 mmol) in dioxane (5 mL) is added in one portion. Refluxing is continued for 48 h and the reaction mixture is allowed to cool to rt. The mixture is concentrated, adsorbed directly onto silica gel (5 g) and subjected to silica gel chromatography (230-400 mesh, 40 g, elution with 1, 2 and 3% MeOH/DCM) to give 84 mg (30%) of 8-(bromomethyl)-7-methyl-10-(3-phenylpropyl)benzo[g]pteridine-2,4(3H,10H)-dione as a solid. ¹H NMR (400 MHz, CDCl₃) δ 2.22 (2H, m), 2.52 (3H, m), 2.88 (2H, t), 4.34 (2H, s), 4.64 (2H, m), 6.84 (1H, s), 7.22-7.45 (5H, m), 8.06 (1H, s), 8.74 (1H, br. s); MS (ESI⁺) for C₂₁H₁₉BrN₄O₂ m/z 439.0 and 441.0 (M+H)⁺; HPLC retention time: 3.74 min. (Method D).

### Step 2 Preparation of 8-[2,6-dimethylmorpholin-4-yl)methyl]-7-methyl-10-(3-phenylpropyl)benzo[g]pteridine-2,4(3H,10H)-dione

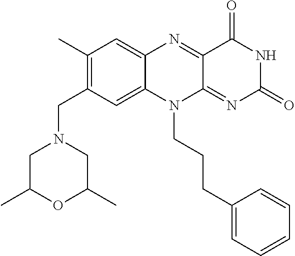 [SMILES: Cc1cc2nc3c(=O)[nH]c(=O)nc-3n(CCCc3ccccc3)c2cc1CN1CC(C)OC(C)C1]

To a well-stirred solution of 8-(bromomethyl)-7-methyl-10-(3-phenylpropyl)benzo[g]pteridine-2,4(3H,10H)-dione (30 mg, 0.07 mmol) in THF (3.0 mL, 37 mmol) is added 2,6-dimethylmorpholine (17 uL, 0.14 mmol). The reaction is stirred at rt under nitrogen for 4 h, diluted with DCM (50 mL) and is washed with saturated, aqueous sodium bicarbonate (20 mL). The layers are separated and the aqueous layer is extracted with DCM (2×20 mL). The organic layers are combined, dried with anhydrous sodium sulfate and concentrated. The residue is triturated with diethyl ether and the solid collect by filtration to give 16 mg (50%) of 8-[(2,6-dimethylmorpholin-4-yOmethyl]-7-methyl-10-(3-phenylpropyl)benzo[g]pteridine-2,4(3H,10H)-dione as a red solid. ¹H NMR (400 MHz, DMSO-d6) δ 1.01 (6H, m), 2.02 (2H, m), 2.47 (3H, s), 2.67 (2H, m), 2.79 (3 H, m), 3.55 (4H, m), 7.23 (5H, m), 7.64 (1H, s), 7.92 (1H, s), 11.34 (1H, s); MS (ESI⁺) for C₂₇H₃₁N₅O₃ m/z 474.3 (M+H)⁺; HPLC retention time: 2.65 min. (Method D).

### Example 19

### [7-Methyl-2,4-dioxo-10-(3-phenylpropyl)-2,3,4,10-tetrahydrobenzo[g]pteridin-8-yl]methyl acetate

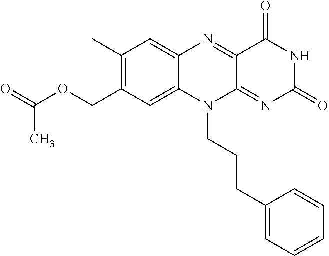 [SMILES: CC(=O)OCc1cc2c(cc1C)nc1c(=O)[nH]c(=O)nc-1n2CCCc1ccccc1]

Sodium acetate (0.0140 g, 0.171 mmol) is added to a mixture of 8-(bromomethyl)-7-methyl-10-(3-phenylpropyl)benzo[g]pteridine-2,4(3H,10H)-dione (0.0500 g, 0.114 mmol) in 5 mL of DMF and the mixture is stirred at rt overnight. The DMF is evaporated and the product is purified by preparative TLC on a 0.5 mm silica gel plate in 50% EtOAc/hexane to give 3.8 mg (8%) of [7-methyl-2,4-dioxo-10-(3-phenylpropyl)-2,3,4,10-tetrahydrobenzo[g]pteridin-8-yl]methyl acetate as red solid. ¹H NMR (400 MHz, CDCl₃) δ 8.55 (s, 1H), 8.11 (s, 1H), 7.33 (d, 3H), 7.34 (m, 2H), 7.25 (s, 1H), 5.17 (s, 2H), 4.75 (br s, 2H), 2.91 (t, 2H), 2.52 (s, 3H), 2.25 (d, 2H), 2.17 (s, 3H); MS (ESI+) for C₂₃H₂₂N₄O₄ m/z 419.2 (M+H)⁺; HPLC retention time: 3.41 min. (System D).

### Example 20

### 7-Methyl-10-(3-phenylpropyl)-8-propylbenzo[g]pteridine-2,4(3H,10H)-dione

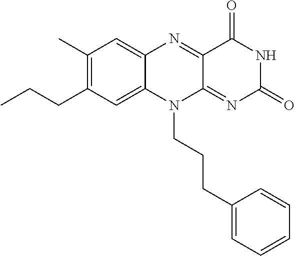 [SMILES: CCCc1cc2c(cc1C)nc1c(=O)[nH]c(=O)nc-1n2CCCc1ccccc1]

### Step 1 Preparation of 5-chloro-4-methyl-2-nitro-N-(3-phenylpropyl)aniline

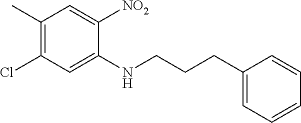 [SMILES: Cc1cc([N+](=O)[O-])c(NCCCc2ccccc2)cc1Cl]

A stirring, N₂ flushed solution of 4-amino-2-chloro-5-nitrotoluene (1.03 g, 5.36 mmol) in dry DMF (10 mL) is cooled in an ice bath. To this solution is added sodium hydride (0.21 g, 5.3 mmol), followed at 1 h with 1-bromo-3-phenylpropane (0.815 mL, 5.36 mmol). After 18 h, additional sodium hydride (0.02 g, 0.5 mmol) is added. At 21 h, the reaction is quenched with ice (10 g) followed by water (50 mL) and Et₂O (100 mL). The mixture is shaken and the organic layer is washed with water (3×30 mL) and concentrated to provide red oil. The residue is chromatographed on silica gel using 3% EtOAc/heptane to give 5-chloro-4-methyl-2-nitro-N-(3-phenylpropyl)aniline (1.36 g, 83%) as a red oil that slowly solidified on standing. ¹H NMR (300 MHz, CDCl₃) δ 8.05 (s, 1H), 7.94 (br s, 1H), 7.27 (m, 5H), 6.81 (s, 1H), 3.27 (m, 2H), 2.79 (m, 2H), 2.29 (s, 3H), 2.07 (m, 2H); MS (ESI+) for C₁₆H₁₇ClN₂O m/z 305.09 (M+H)⁺.

### Step 2 Preparation of 4-methyl-2-nitro-N-(3-phenylpropyl)-5-propylaniline

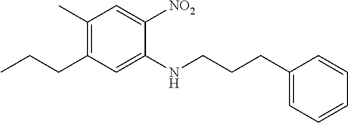 [SMILES: CCCc1cc(NCCCc2ccccc2)c([N+](=O)[O-])cc1C]

To an N₂ flushed 40 mL vial containing 5-chloro-4-methyl-2-nitro-N-(3-phenylpropyl)aniline (0.355 g, 1.1 mmol) and 1,4-dioxane (25 mL) is added Cs₂CO₃ (1.82 g, 5.5 mmol), KF (0.254 g, 4.4 mmol), propylboronic acid (0.392 g, 4.5 mmol), and bis(tri-tert-butylphosphine)palladium(0) (0.096 g, 0.18 mmol). The reaction mixture is shaken at 100° C. for 24 h. The mixture is cooled to rt and filtered through a silica gel column (2×4 cm), using DCM (50 mL). The filtrate is concentrated and chromatographed on silica gel (4×14 cm column, using a gradient from heptane to 20% DCM/heptane) to provide 165 mg (48%) of 4-methyl-2-nitro-N-(3-phenylpropyl)-5-propylaniline as a red oil. MS (ESI+) for C₁₉H₂₄N₂O₂ m/z 313.3 (M+H)⁺.

### Step 3 Preparation of 4-methyl-N-(3-phenylpropyl)-5-propylbenzene-1,2-diamine

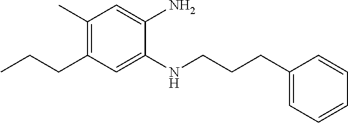 [SMILES: CCCc1cc(NCCCc2ccccc2)c(N)cc1C]

A slurry of 4-methyl-2-nitro-N-(3-phenylpropyl)-5-propylaniline (0.081 g, 0.26 mmol), EtOH (40 mL), and Raney Nickel (100 mg) is flushed with N₂ and then stirred under 1 atmosphere of H₂ for 18 h. The mixture is filtered through Solka Floc® (5×5 mL EtOH rinses) and concentrated to give 4-methyl-N-(3-phenylpropyl)-5-propylbenzene-1,2-diamine (52 mg, 71%) as a white solid. MS (ESI+) for C₁₉H₂₆N m/z 283.29 (M+H)⁺. HPLC retention time 3.84 min. (method D).

### Step 4 Preparation of 7-methyl-10-(3-phenylpropyl)-8-propylbenzo[g]pteridine-2,4(3H,10H)-dione

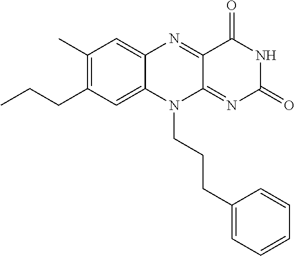 [SMILES: CCCc1cc2c(cc1C)nc1c(=O)[nH]c(=O)nc-1n2CCCc1ccccc1]

To a mixture of 4-methyl-N-(3-phenylpropyl)-5-propylbenzene-1,2-diamine (0.052 g, 0.18 mmol), alloxan (29 mg, 0.18 mmol) and boric acid (34.6 mg, 0.560 mmol) is added acetic acid (5.1 mL, 9 mmol). The reaction is flushed with N₂ and stirred at rt for 2 h. The reaction mixture is then mixed with toluene (5 mL) and evaporated to dryness. The residue is dissolved in DCM (30 mL) and filtered through Celite. The filtrate is washed with water (1×10 mL) and aqueous NaHCO₃ (10 mL), and the organic layer is concentrated. This residue is chromatographed on silica gel (1% MeOH/DCM) to give 7-methyl-10-(3-phenylpropyl)-8-propylbenzo[g]pteridine-2,4(3H,10H)-dione (31 mg; 43%) as an orange solid: ¹H NMR (300 MHz, DMSO-d₆) δ 11.32 (s, 1H), 7.90 (s, 1H), 7.29 (m, 6H), 4.59 (m, 2H), 2.80 (m, 2H), 2.71 (m, 2H), 2.42 (s, 3H), 2.03 (m, 2H), 1.56 (m, 2H), 0.98 (t, 3H); MS (ESI+) for C₂₃H₂₄N₄O₂ m/z 389.26 (M+H)⁺; HPLC retention time 4.08 min. (method D).

### Example 21

### 7,8-Dimethyl-10-(2-(phenylsulfonyyl)ethyl)benzo[g]pteridine-2,4(3H,10H)-dione

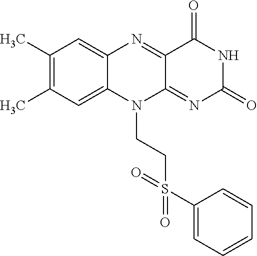 [SMILES: Cc1cc2nc3c(=O)[nH]c(=O)nc-3n(CCS(=O)(=O)c3ccccc3)c2cc1C]

To a stirred mixture of 7,8-dimethyl-10-[2-(phenylthio)ethyl]benzo-[g]pteridine-2,4(3H,10H)-dione (0.0760 g, 0.201 mmol) and DCM (20 mL) under nitrogen is added a solution of MCPBA (0.0900 g, 0.402 mmol) in EtOH (2 mL). At 3 h, additional MCPBA (0.020 g, 0.089 mmol) is added as a solution in EtOH (1 mL). After 45 minutes, aqueous NaHCO₃ (8 mL) is added, and the reaction mixture is stirred for 1 h and filtered. The solid is washed successively with water (3×5 mL), ethanol (2×2 mL) and DCM (2×3 mL) and dried in vacuo to give 7,8-dimethyl-10-[2-(phenylsulfonyl)ethyl]benzo[g]pteridine-2,4(3H,10H)-dione (45 mg, 52%) as an orange solid. ¹H NMR (DMSO-d₆) δ 11.38 (s, 1 H), 7.97-7.64 (m, 6H), 7.35 (s, 1H), 4.84 (m, 2H), 3.87 (m, 2H), 2.42 (s, 3H), 2.39 (s, 3 H); MS (ESI−) for C₂₀H₁₈N₄O₄S m/z 409.08 (M−H)⁻.

### Example 22

### 10-(3-(4-Chlorophenyl)-2-hydroxypropyl)-8-methylbenzo[g]pterodome-2,4(3H,10H)-dione

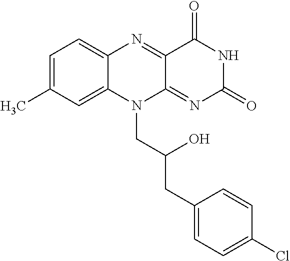 [SMILES: Cc1ccc2nc3c(=O)[nH]c(=O)nc-3n(CC(O)Cc3ccc(Cl)cc3)c2c1]

### Step 1 Preparation of 1-(4-Chlorophenyl)-3-[(5-methyl-2-nitrophenyl)amino]-propan-2-ol

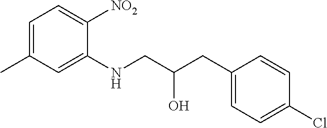 [SMILES: Cc1ccc([N+](=O)[O-])c(NCC(O)Cc2ccc(Cl)cc2)c1]

A slurry of 2-(4-chlorobenzyl)oxirane (0.280 g, 1.66 mmol), 5-methyl-2-nitroaniline (0.277 g, 1.82 mmol) and ytterbium(III) triflate (0.206 g, 0.33 mmol) in dry CH₃CN (10 mL) is stirred at rt for 18 h. The reaction is concentrated and chromatographed on silica gel using 8% EtOAc/heptanes to provide 1-(4-chlorophenyl)-3-[(5-methyl-2-nitrophenyl)amino]propan-2-ol (0.31 g, 58%) as a red oil. MS (ESI+) for C₁₆H₁₇ClN₂O₃ m/z 321.02 (M+H)⁺.

### Step 2 Preparation of 1-[(2-amino-5-methylphenyl)amino]-3-(4-chlorophenyl)propan-2-ol

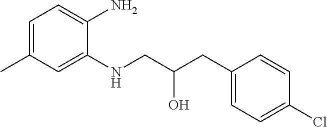 [SMILES: Cc1ccc(N)c(NCC(O)Cc2ccc(Cl)cc2)c1]

A stirred mixture of 1-(4-chlorophenyl)-3-[(5-methyl-2-nitrophenyl)amino]propan-2-ol (0.209 g, 0.652 mmol), EtOH (20 mL), and Raney Nickel (100 mg) is flushed with N₂ and then stirred under 1 atm of H₂ (balloon). After overnight stirring, the mixture is filtered through Solka Floc® (5×5 mL EtOH rinses) and the filtrate is evaporated to give 1-[(2-amino-5-methylphenyl)amino]-3-(4-chlorophenyl)propan-2-ol (0.114 g, 60%) as a white solid. MS (ESI+) for C₁₆H₁₉ClN₂O m/z 291.04 (M+H)⁺.

### Step 3 Preparation of 10-(3-(4-chlorophenyl)-2-hydroxypropyl)-8-methylbenzo-[g]pteridine-2,4(3H,10H)-dione

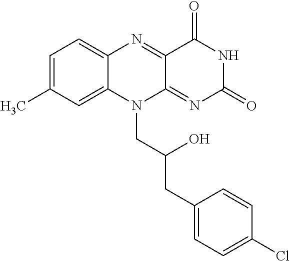 [SMILES: Cc1ccc2nc3c(=O)[nH]c(=O)nc-3n(CC(O)Cc3ccc(Cl)cc3)c2c1]

To a well-stirred mixture of 1-[(2-amino-5-methylphenyl)amino]-3-(4-chlorophenyl)propan-2-ol (0.1136 g, 0.3907 mmol), alloxan (65.67 mg, 0.4102 mmol) and boric acid (72.47 mg, 1.172 mmol) under nitrogen is added AcOH (7 mL). After 72 h, the reaction mixture is filtered and the solid is washed successively with AcOH (4×2 mL), water (5×15 mL) and 90% MeOH/DCM (4×40 mL). The solid is dried at 60° C. under high vacuum to give (10-[3-(4-chlorophenyl)-2-hydroxypropyl]-8-methylbenzo[g]pteridine-2,4(3H,10H)-dione (0.125 g, 78%) as a yellow solid. ¹H NMR (DMSO-d₆) δ 11.39 (s, 1H), 7.99 (d, 1H), 7.73 (s, 1H), 7.47 (d, 1H), 7.33 (m, 4H), 5.02 (m, 1H), 4.64 (m, 2H), 4.30 (m, 1H), 2.90 (m, 2H), 2.55 (s, 3H); MS (ESI+) for C₂₀H₁₇ClN₄O₃ m/z 397.14 (M+H)⁺.

### Example 23

### N-{10-[3-(4-Chlorophenyl)propyl]-7-methyl-2,4-dioxo-2,3,4,10-tetrahydrobenzo[g]-pteridin-8-yl}propanamide

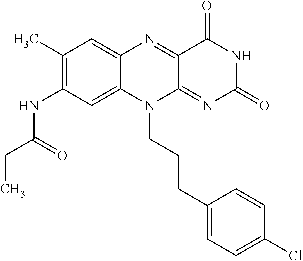 [SMILES: CCC(=O)Nc1cc2c(cc1C)nc1c(=O)[nH]c(=O)nc-1n2CCCc1ccc(Cl)cc1]

Step 1 Preparation of N-[3-(4-chlorophenyl)propyl]-4-methyl-3-nitroaniline

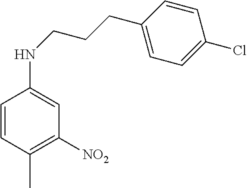 [SMILES: Cc1ccc(NCCCc2ccc(Cl)cc2)cc1[N+](=O)[O-]]

A N₂ flushed solution of 4-methyl-3-nitro-aniline (1.51 g, 9.92 mmol), 1-(3-bromopropyl)-4-chlorobenzene (1.31 g, 5.61 mmol), and DIPEA (3 mL) is shaken at 70° C. for 18 h. The reaction is concentrated and chromatographed on silica gel using 2% EtOAc/heptane to provide N-[3-(4-chlorophenyl)propyl]-4-methyl-3-nitroaniline (1.34 g; 78%) as an orange solid. MS (ESI+) for C₁₆H₁₇ClN₂O₂ m/z 305.2 (M+H)⁺.

### Step 2 Preparation of N1-[3-(4-chlorophenyl)propyl]-4-methylbenzene-1,3-diamine

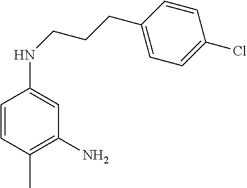 [SMILES: Cc1ccc(NCCCc2ccc(Cl)cc2)cc1N]

A stirred mixture of N-[3-(4-chlorophenyl)propyl]-4-methyl-3-nitroaniline (1.34 g, 4.41 mmol), EtOH (40 mL), and Raney Nickel (100 mg) is stirred under 1 atmosphere of H₂ (balloon). After 18 h, the mixture is filtered through Solka Floc® (5×5 mL EtOH rinses) and the filtrate is concentrated to provide N1-[3-(4-chlorophenyl)propyl]-4-methylbenzene-1,3-diamine (1.23 g, 96%) as a white solid. MS (ESI+) for C₁₆H₁₉ClN₂ m/z 275.20 (M+H)⁺.

### Step 3 Preparation of 8-amino-10-[3-(4-ehlorophenvnpropyl]-7-methylbenzo[g]pteridine-2,4(3H,10H)-dione

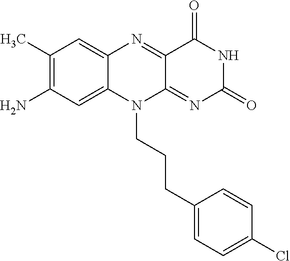 [SMILES: Cc1cc2nc3c(=O)[nH]c(=O)nc-3n(CCCc3ccc(Cl)cc3)c2cc1N]

To a well-stirred mixture of N1-[3-(4-chlorophenyl)propyl]-4-methylbenzene-1,3-diamine (1.23 g, 4.25 mmol) and violuric acid monohydrate (740 mg, 4.2 mmol) is added AcOH (70 mL). The reaction is flushed with N₂, heated at 115° C. for 45 min. and is allowed to cool to rt overnight. The precipitate is collected by filtration, washed with AcOH (5×2 mL), DCM (3×20 mL) and dried overnight under high vacuum at 55° C. to provide 8-amino-10-[3-(4-chlorophenyl)propyl]-7-methylbenzo[g]pteridine-2,4(3H,10H)-dione (1.399 g, 83%) as a red solid. ¹H NMR (300 MHz, DMSO-d₆) δ 10.93 (s, 1H), 7.66 (s, 1 H), 7.32 (m, 4H), 7.19 (br s, 2H), 6.77 (s, 1H), 4.48 (m, 2H), 2.77 (m, 2H), 2.23 (s, 3 H), 2.02 (m, 2H); MS (ESI+) for C₂₀H₁₈ClN₅O₂ m/z 396.19 (M+H)⁺.

### Step 4 Preparation of N-{10-[3-(4-chlorophenyl)propyl]-7-methyl-2,4-dioxo-2,3,4,10-tetrahydrobenzo[g]pteridin-8-yl}propanamide

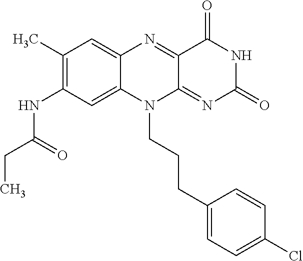 [SMILES: CCC(=O)Nc1cc2c(cc1C)nc1c(=O)[nH]c(=O)nc-1n2CCCc1ccc(Cl)cc1]

To a well-stirred mixture of 8-amino-10-[3-(4-chlorophenyl)propyl]-7-methylbenzo[g]pteridine-2,4(3H,10H)-dione (0.071 g, 0.18 mmol) and DMF (5 mL) is added propanoyl chloride (2 mL, 20 mmol). After shaking overnight at rt, the temperature is increased to 40° C. and shaking is continued for 48 h. The mixture is quenched with ice water (5 mL), shaken for 30 min. and filtered. The solids are washed with water (3×2 mL) and MeOH (6×2 mL) and dried at 70° C. under high vacuum to give N-{10-[3-(4-chlorophenyl)propyl]-7-methyl-2,4-dioxo-2,3,4,10-tetrahydrobenzo[g]-pteridin-8-yl}propanamide (63 mg, 76%) as an orange solid. ¹H NMR (DMSO-d₆) δ 11.29 (s, 1H), 9.55 (s, 1H), 8.37 (s, 1H), 7.97 (s, 1H), 7.31 (s, 4H), 4.55 (m, 2H), 2.78 (m, 2H), 2.55 (m, 2H), 2.44 (s, 3H), 2.08 (m, 2H), 1.16 (t, 3H); MS (ESI+) for C₂₃H₂₂ClN₅O₃ m/z 452.19 (M+H)⁺.

### Example 24

### 7,8-Dimethyl-10-(1-methyl-3-phenylpropyl)benzo[g]pteridine-2,4(3H,10H)-dione

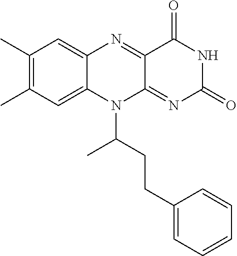 [SMILES: Cc1cc2nc3c(=O)[nH]c(=O)nc-3n(C(C)CCc3ccccc3)c2cc1C]

### Step 1 Preparation of 3-bromobutyl)benzene

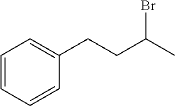

To a cold (0° C.), well-stirred solution of triphenylphosphine dibromide [freshly prepared from bromine (0.686 mL, 13.3 mmol) and triphenylphosphine (3.49 g, 13.3 mmol)] in 30 mL of CH₂Cl₂ is added 4-phenylbutan-2-ol (2.11 mL, 13.3 mmol) dropwise as a solution in 10 mL of CH₂Cl₂. The ice bath is allowed warm to rt overnight, diluted with heptane (60 mL) and filtered. The solids are washed with heptane (4×15 mL) and the filtrates are combined, concentrated and chromatographed on silica gel, using heptane as the eluent, to give (3-bromobutyl)benzene a colorless liquid (1.93 g, 68%). HPLC retention time 5.25 min. (method D).

### Step 2 Preparation of 4,5-dimethyl-N-(1-methyl-3-phenylpropyl)benzene-1,2-diamine

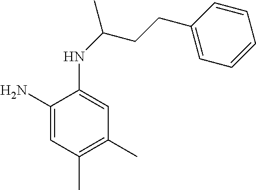 [SMILES: Cc1cc(N)c(NC(C)CCc2ccccc2)cc1C]

A well-stirred slurry of (3-bromobutyl)benzene (0.400 g, 1.88 mmol), 4,5-dimethyl-o-phenylenediamine (1.02 g, 7.51 mmol), tetra-n-butylammonium iodide (0.0693 g, 0.188 mmol) and sodium bicarbonate (0.315 g, 3.75 mmol) in dry toluene (30 mL) is heated at 100° C. After 47 h, additional tetra-n-butylammonium iodide (0.040 g, 0.11 mmol) is added and stirring is continued for 2 h. The reaction mixture is cooled to rt, partitioned between water and toluene (100 mL each). EtOAc (20 mL) is added and the organic layer is washed with water (3×40 mL) and concentrated. The residue is subjected to silica gel chromatography (230-400 mesh, 150 g, elution with 15% ethyl acetate/heptane) to give the 4,5-dimethyl-N-(1-methyl-3-phenylpropyl)benzene-1,2-diamine (0.120 g; 23%) as a reddish brown oil. MS (ESI+) for C₁₈H₂₄N₂ m/z 269.20 (M+H)⁺.

### Step 3 Preparation of 7,8-dimethyl-10-(1-methyl-3-phenylpropyl)benzo[g]pteridine-2,4(3H,10H)-dione

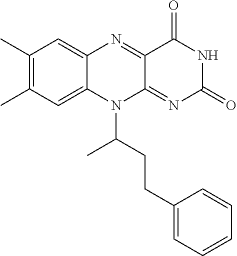 [SMILES: Cc1cc2nc3c(=O)[nH]c(=O)nc-3n(C(C)CCc3ccccc3)c2cc1C]

To a mixture of 4,5-dimethyl-N-(1-methyl-3-phenylpropyl)benzene-1,2-diamine (0.109 g, 0.406 mmol), alloxan (68.26 mg, 0.4264 mmol) and boric acid (75.33 mg, 1.218 mmol) is added acetic acid (8 mL, 100 mmol). The reaction mixture is flushed N₂ and stirred at rt for 18 h. The reaction mixture is then azeotroped with toluene (2×30 mL), and the residue is slurried with DCM (80 mL), filtered and the filtrate washed with water (20 mL). The organic layer is concentrated and dried overnight under high vacuum to give 7,8-dimethyl-10-(1-methyl-3-phenylpropyl)benzo[g]pteridine-2,4(3H,10H)-dione (0.120 g, 78%) as a yellow solid. ¹H NMR (300 MHz, DMSO-d₆) δ 11.29 (m, 1H), 7.80 (m, 2H), 7.09 (m, 5 H), 5.21-6.33 (m, 1H), 2.94 (m, 1H), 2.43 (s, 3H), 2.38 (s, 3H), 1.68 (m, 3H); MS (ESI+) for C₂₂H₂₂N₄O₂ m/z 375.09 (M+H)⁺.

### Examples 25 and 26

### (10Z)-14-methyl-1,17,20,22-tetraazapentacyclo[11.10.2.25,8.016,24.018,23]heptacosa-5,7,10,13(25),14,16(24),17,22,26-nonaene-19,21-dione

and

### (10E)-14-methyl-1,17,20,22-tetraazapentacyclo[11.10.2.25,8.016,24.018,23]heptacosa-5,7,10,13(25),14,16(24),17,22,26-nonaene-19,21-dione

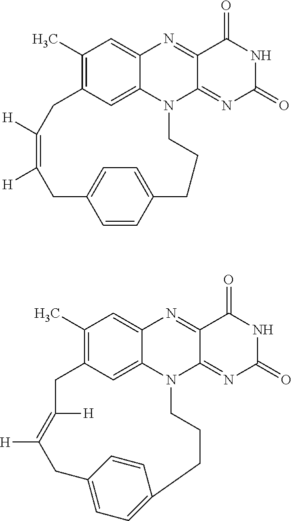 [SMILES: Cc1cc2nc3c(=O)[nH]c(=O)nc-3n3c2cc1C/C=C\Cc1ccc(cc1)CCC3 ; Cc1cc2nc3c(=O)[nH]c(=O)nc-3n3c2cc1C/C=C/Cc1ccc(cc1)CCC3]

### Step 1: Preparation of 2-allyl-1-methyl-4-nitrobenzene

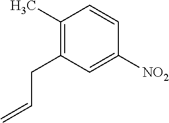

A mixture of 2-bromo-1-methyl-4-nitrobenzene (2.42 g, 11.2 mmol), allyltributylstannane (4.26 g, 12.8 mmol) and tetrakis(triphenylphosphine)palladium(0) (480 mg, 0.04 mmol) in DMF (15 mL) is heated at 176° C. for 20 min. in a microwave. The solid is removed by filtration through a celite pad and the pad is washed with EtOAc. The filtrate is evaporated and the residue is purified by flash column chromatography using a gradient from 0 to 5% EtOAc in hexane as eluent. The product is isolated (1.6 g, 80%) as a green oil. ¹H-NMR (400 MHz, DMSO-d6): δ 2.38 (s, 3H), 3.50 (d, 2H), 5.06 (d, 1H), 5.13 (d, 1H), 5.98 (ddt, 1H), 7.46 (s, 1H), 7.01 (m, 2H).

### Step 2: Preparation of 3-allyl-4-methylaniline

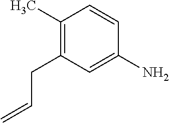

Acetic acid (19 mL) is slowly added to a round bottom flask charged with 2-allyl-1-methyl-4-nitrobenzene (3.39 g, 19.1 mmol) and zinc dust (12.5 g, 191 mmol) in DCM (260 mL) at 0° C. After 30 min., the reaction mixture is filtered through celite and washed liberally with DCM. Saturated, aqueous NaHCO₃ (30 mL) is added to the filtrate, and the organic layer is dried over Na₂SO₄ and then concentrated. The desired product is isolated (2.98 g) as orange oil. ¹H-NMR (400 MHz, CDCl₃): δ 2.20 (s, 3H), 3.30 (d, 2H), 3.53 (s, 2H), 5.04 (d, 1H), 5.06 (d, 1H), 5.95 (ddt, 1H), 6.51 (dd, 1H), 6.54 (d, 1H), 6.95 (d, 1H). LC-MS m/z 148.0 [M+H]⁺.

### Step 3: Preparation of methyl 3-(4-bromophenyl)propanoate

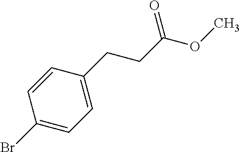

A mixture of 3-(4-bromophenyl)propanoic acid and sulfuric acid (1 mL) is refluxed in methanol (100 mL) for 3 h. The reaction is made basic with saturated, aqueous Na₂CO₃ and is extracted with DCM. The organic layer is dried over Na₂SO₄, filtered, and concentrated under reduced pressure to obtain a crude product (4.25 g) as yellow oil. This material is used in the next step without further purification. ¹H-NMR (400 MHz, DMSO-d6): δ 2.62 (t, 2H), 2.82 (t, 2H), 3.58 (s, 3H), 7.20 (d, 2H), 7.46 (d, 2H).

### Step 4: Preparation of methyl 3-(4-allylphenyl)propanoate

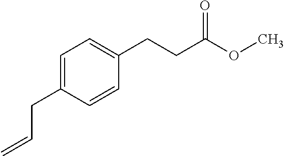

A mixture of methyl 3-(4-bromophenyl)propanoate (2.43 g, 10 mmol), potassium allyltrifluoroborate (1.63 g, 11 mmol), 1,1′-bis(di-tert-butylphosphino)ferrocene (1.42, 3 mmol), palladium (II) acetate (0.337 g, 1.5 mmol) and anhydrous K₂CO₃ (4.14 g, 30 mmol) is refluxed in anhydrous THF (100 mL) for 16 h. The reaction mixture is filtered through a celite pad and washed with EtOAc. The filtrate is evaporated and the residue is purified by flash column chromatography using a gradient from 0 to 20% EtOAc in hexane as the eluent. The desired product is isolated (1.54 g, 76%) as a green oil. ¹H-NMR (400 MHz, CDCl₃): δ 2.64 (t, 2H), 2.95 (t, 2H), 3.38 (d, 2H), 3.69 (s, 3H), 5.08 (d, 1H), 5.11 (d, 1H), 5.99 (ddt, 1H), 7.14 (m, 4H).

### Step 5: Preparation of 3-(4-allylphenyl)propan-1-ol

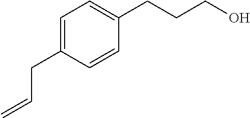

LAH (361 mg, 9.5 mmol) is added over 15 min. to a solution of methyl 3-(4-allylphenyl)propanoate (1048 mg, 5.1 mmol) in diethyl ether (100 mL) at 0° C. for 2 h. The reaction mixture is quenched with water at 0° C., the organic layer is collected, dried over Na₂SO₄, and concentrated under vacuum. The desired product is obtained (911 mg) as a colourless oil. ¹H-NMR (400 MHz, CDCl₃): δ 2.47 (m, 2H), 2.71 (t, 2H), 3.39 (d, 2H), 3.70 (d, 2H), 5.08 (d, 1H), 5.11 (d, 1H), 5.98 (ddt, 1H), 7.14 (m, 4H).

### Step 6: Preparation of 1-allyl-4-(3-bromopropyl)benzene

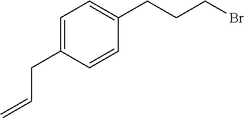

Triphenyl phosphine (1.56 g, 5.93 mmol) is added to a solution of 3-(4-allylphenyl)propan-1-ol (0.82 g, 4.63 mmol) and carbon tetrabromide (4.7 g, 14.1 mmol) in DCM (100 mL) at 0° C. The reaction is then slowly warmed to room temperature and stirred for a further 2 h. The reaction mixture is then concentrated, dry loaded on silica gel and purified by flash column chromatography using a gradient from 0 to 5% EtOAc in hexane as eluent. The desired product is obtained (0.942 g, 85% over two steps) as a colourless oil. ¹H NMR (400 MHz, CDCl₃): δ 2.18 (m, 2H), 2.78 (t, 2H), 3.41 (m, 4H), 5.09 (d, 1H), 5.11 (d, 1H), 5.99 (ddt, 1H), 7.15 (s, 4H).

### Step 7: Preparation of 3-allyl-N-(3-(4-allylphenyl)propyl)-4-methylaniline

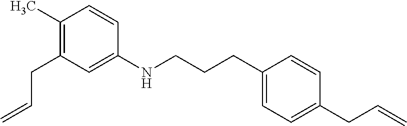 [SMILES: C=CCc1ccc(CCCNc2ccc(C)c(CC=C)c2)cc1]

A solution of 1-allyl-4-(3-bromopropyl)benzene (1.1 g, 4.8 mmol) and 3-allyl-4-methylaniline (1.2 g, 8.15 mmol) in DIPEA (0.68 g, 5.4 mmol) is heated at 100° C. for 3 h. The reaction mixture is then concentrated, dry loaded on silica gel and purified by flash column chromatography using a gradient from 0 to 30% Et₂O in hexane as eluent. The desired product is isolated (1.05 g, 75% yield) as an orange oil. LC-MS m/z 306.2 [M+H]⁺.

### Step 8: Preparation of 64(3-allyl-4-methylphenyl)(3-(4-allylphenyl)propynamino)pyrimidine-2,4(1H,3H)-dione

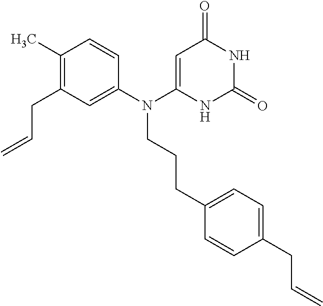 [SMILES: C=CCc1ccc(CCCN(c2ccc(C)c(CC=C)c2)c2cc(=O)[nH]c(=O)[nH]2)cc1]

A solution of 3-allyl-N-(3-(4-allylphenyl)propyl)-4-methylaniline (0.87 g, 2.8 mmol), DIPEA (0.5 mL, 2.8 mmol) and 6-chlorouracil (1.1 g, 7.6 mmol) in DMF (12 mL) in a microwave vial is set to 175° C. for 30 min. EtOAc is added and the organic phase is washed with water, then brine. The organic layer is dried over Na₂SO₄ and dried loaded on silica gel. The crude product is purified by flash column chromatography using a gradient from 0 to 20% EtOAc in hexane as the eluent, followed by 0 to 10% MeOH in DCM to obtain desired product (297 mg, 25%) as a yellow solid. LC-MS m/z 416.2 (M+H)⁺.

### Step 9: Preparation of 8-allyl-10-(3-(4-allylphenyl)propyl)-7-methyl-2,4-dioxo-2,3,4,10-tetrahydrobenzoMpteridine 5-oxide

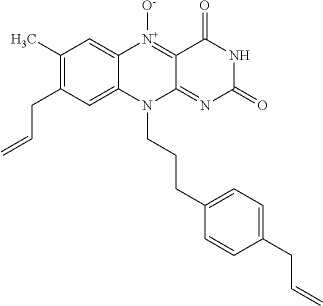 [SMILES: C=CCc1ccc(CCCn2c3nc(=O)[nH]c(=O)c-3[n+]([O-])c3cc(C)c(CC=C)cc32)cc1]

6-((3-Allyl-4-methylphenyl)(3-(4-allylphenyl)propyl)amino)pyrimidine-2,4(1H,3H)-dione (329 mg, 0.79 mmol) and sodium nitrite (277 mg, 4 mmol) is dissolved in acetic acid (7 mL) and stirred at room temperature for 45 min. The reaction mixture is then concentrated under vacuum and the crude product (orange solid) is used in the next step. LC-MS m/z 443.1 [M+H]⁺.

### Step 10: Preparation of 8-allyl-10-(3-(4-allylphenyl)propyl)-7-methylbenzo[g]pteridine-2,4(3H,10H)-dione

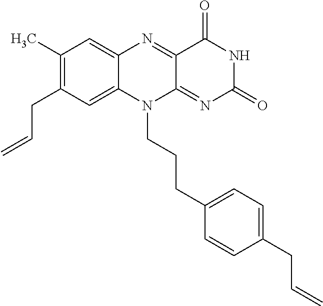 [SMILES: C=CCc1ccc(CCCn2c3nc(=O)[nH]c(=O)c-3nc3cc(C)c(CC=C)cc32)cc1]

8-Allyl-10-(3-(4-allylphenyl)propyl)-7-methyl-2,4-dioxo-2,3,4,10-tetrahydrobenzo[g]pteridine 5-oxide (0.79 mmol) is dissolved in EtOH (100 mL), and TEA (1 mL) is added, followed by a solution of Na₂S₂O₄ (280 mg, 1.6 mmol) in water (20 mL). The resulting solution is stirred at room temperature for 15 min. The reaction mixture is then concentrated under vacuum and purified by preparative HPLC (Method M) to obtain product (35.2 mg, 10%) as a yellow solid. LC-MS m/z 427.2 [M+H]⁺.

### Step 11: Preparation of (10Z)-14-methyl-1,17,20,22-tetraazapentacyclo[11.10.2.25,8.016,24.018,23]heptacosa-5,7,10,13(25),14,16(24),17,22,26-nonaene-19,21-dione and (10E)-14-methyl-1,17,20,22-tetraazapentacyclo[11.10.2.25,8.016,24.018,23]heptacosa-5,7,10,13(25),14,16(24),17,22,26-nonaene-19,21-dione

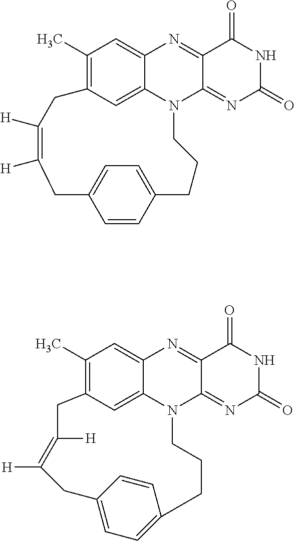 [SMILES: Cc1cc2nc3c(=O)[nH]c(=O)nc-3n3c2cc1C/C=C\Cc1ccc(cc1)CCC3 ; Cc1cc2nc3c(=O)[nH]c(=O)nc-3n3c2cc1C/C=C/Cc1ccc(cc1)CCC3]

Dry toluene (1200 mL) is refluxed in a round bottom flask equipped with a Dean-Stark condenser for 30 min. to remove excess water. Upon removing the Dean-Stark condenser, 8-allyl-10-(3-(4-allylphenyl)propyl)-7-methylbenzo[g]pteridine-2,4(3H,10H)-dione (30 mg, 0.07 mmol) in DCM (5 mL) and Grubbs 1 reagent (20 mg, 0.02 mmol) in toluene (5 mL) are added at reflux simultaneously. The mixture is stirred for a further 20 min. and quenched with DMSO (4 mL), cooled to room temperature, concentrated under vacuum, and purified by preparative HPLC (Method N) to obtain the desired products.

### Data for Example 25

(10Z)-14-methyl-1,17,20,22-tetraazapentacyclo[11.10.2.25,8.016,24.018,23]heptacosa-5,7,10,13(25),14,16(24),17,22,26-nonaene-19,21-dione: yellow solid, ¹H NMR (400 MHz, DMSO-d6): δ 1.06 (br s, 2H), 2.43 (s, 3H), 2.68 (br s, 1H), 2.99 (br s, 1H), 3.49 (s, 2H), 3.67 (br s, 1H), 4.55 (br s, 1H), 5.09 (s, 1H), 5.54 (dt, J=7.5, 7.5 Hz, 1H), 6.14 (dt, 1H), 7.39 (m, 4H), 7.83 (s, 1H), 11.32 (s, 1H); LC-MS m/z 399.1 [M+H].

### Data for Example 26

(10E)-14-methyl-1,17,20,22-tetraazapentacyclo[11.10.2.25,8.016,24.018,23]heptacosa-5,7,10,13(25),14,16(24),17,22,26-nonaene-19,21-dione: yellow solid, ¹H NMR (400 MHz, DMSO-d6): δ 1.81 (m, 2H), 2.31 (s, 3H), 2.80 (m, 2H), 3.26-3.62 (overlapping signals with water), 4.08 (s, 2H), 5.12 (dt, 1H, J=6.5, 15.5 Hz), 5.92 (s, 1H), 6.18 (dt, 1H), 7.27 (d, 2H), 7.31 (d, 2H), 7.85 (s, 1H), 11.3 (s, 1H). LC-MS m/z 399.1 [M+H].

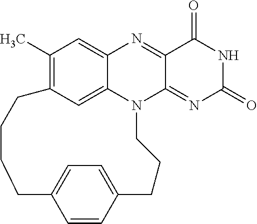 [SMILES: Cc1cc2nc3c(=O)[nH]c(=O)nc-3n3c2cc1CCCCc1ccc(cc1)CCC3]

### Preparation of 14-methyl-1,17,20,22-tetraazapentacyclo[11.10.2.25,8.016,24.018,23]heptacosa-5,7,13(25),14,16(24),17,22,26-octaene-19,21-dione

A solution of (10E)-14-methyl-1,17,20,22-tetraazapentacyclo[11.10.2.25,8.016,24.018,23]heptacosa-5,7,10,13(25),14,16(24),17,22,26-nonaene-19,21-dione (2 mg, 0.005 mmol) in EtOAc (8 mL) is purged with argon for 5 min. A catalytic amount of palladium on carbon (10% wt. on carbon) is added and the reaction mixture is placed under an atmosphere of hydrogen for 4 h. The reaction mixture is filtered through a celite pad and is washed with MeOH until no color is seen. The filtrate is concentrated under reduced pressure to dryness and purified by preparative TLC using 5% MeOH in DCM as the eluent to obtain desired product (0.7 mg, 35%) as a yellow solid. LC-MS m/z 401.1 [M+H]⁺.

### Example 28

### 10-(2-(Benzylamino)-3-phenylpropyl)-7,8-dimethylbenzo[g]pteridine-2,4(3H,10H)-dione

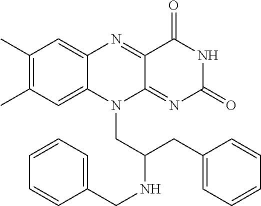 [SMILES: Cc1cc2nc3c(=O)[nH]c(=O)nc-3n(CC(Cc3ccccc3)NCc3ccccc3)c2cc1C]

10-(2-Amino-3-phenylpropyl)-7,8-dimethylbenzo[g]pteridine-2,4(3H,10H)-dione (23 mg, 0.061 mmol) is dissolved in MeOH (5 mL) at room temperature, and then benzaldehyde (7 mg, 0.067 mmol) and AcOH (1 drop) are added. The reaction mixture is stirred at room temperature for 2.5 h, and then NaBH₃CN (8 mg, 0.13 mmol) is added in one portion and the resulting mixture is stirred at room temperature for 4 h. The reaction is quenched with H₂O (caution, 2 mL), and the reaction mixture is evaporated. The crude product is dissolved in DCM:MeOH [4:1] and purified using a preparative TLC plate [5% MeOH/DCM, then 10% MeOH/DCM] to afford the desired product, 10-(2-(benzylamino)-3-phenylpropyl)-7,8-dimethylbenzo[g]pteridine-2,4(3H,10H)-dione as a yellow solid. ¹H NMR (400 MHz, DMSO-d₆) δ 2.10 (3H, t), 2.33 (3H, t), 3.16-3.64 (2H, m's), 3.8-4.0 (1H, m), 4.24-4.48 (2H, m), 4.72-4.88 (1H, m), 5.44-5.64 (1H, m), 6.16-6.28 (1H, m), 7.32-7.56 (11H, m, s), 7.91 (s, 1H), 11.57 (s, 1H); MS (ESI⁺) for C₁₀H₁₂N₂O₂ m/z 466.27 (M+H)⁺.

### Example 29

### 10-[3-(4-Chlorophenyl)-2-isobutoxypropyl]-7-isopropylbenzo[g]pteridine-2,4(3H,10H)-dione

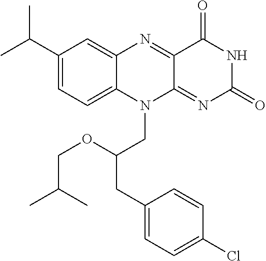 [SMILES: CC(C)COC(Cc1ccc(Cl)cc1)Cn1c2nc(=O)[nH]c(=O)c-2nc2cc(C(C)C)ccc21]

### Step 1 Preparation of Isobutyl Isobutoxyacetate

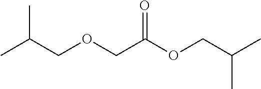

To a well-stirred solution of isobutyl alcohol (50 mL) at rt is added metallic sodium (1.00 g, 43.5 mmol) and the reaction mixture is heated at 50° C. for 18 h. The mixture is cooled to rt and a solidified mass of the sodium salt is obtained. THF (10 mL) is added at rt to give a solution, and tert-butyl α-bromoacetate (7.26 g, 37.2 mmol) is added. The mixture is stirred at rt for 5 h, diluted with 200 mL of water and extracted with hexane (4×150 mL). The combined organic layers are washed with brine, dried with anhydrous sodium sulfate and concentrated. The residue is subjected to atmospheric distillation (bath to 140° C.). The pot contained the product and excess alcohol. This is vacuum distilled at 20 torr @ 100 C to remove the remaining alcohol. The pot contained 4.61 g of isobutyl isobutoxyacetate as a liquid. ¹H NMR (400 MHz, CDCl₃) δ ppm 0.93 (12H, m), 1.94 (2 H, m), 3.30 (2H, d), 3.94 (2H, d), 4.09 (2H, s).

### Step 2 Preparation of isobutyl 3-(4-chlorophenyl)-2-isobutoxyacrylate

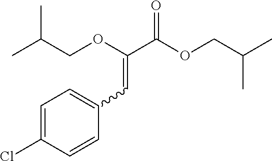 [SMILES: CC(C)COC(=O)C(=Cc1ccc(Cl)cc1)OCC(C)C]

To a cold (0° C. ice bath) well-stirred solution of isobutyl isobutoxyacetate (1.17 g, 6.21 mmol) and 4-chlorobenzaldehyde (0.5824 g, 4.14 mmol) in dry THF (18 mL) is added solid potassium tert-butoxide (0.5579 g, 4.97 mmol) portionwise. After 1 h, the bath is removed and the reaction allowed to warm to rt and is stirred overnight. The reaction is quenched with saturated, aqueous ammonium chloride (5 mL) and diluted with ethyl acetate (100 mL). The layers are separated and the aqueous layer is extracted with ethyl acetate (3×50 mL). The organic layers are combined, dried with anhydrous sodium sulfate and concentrated. The residue is subjected to silica gel chromatography (230-400 mesh, 150 g, elution with 10% ethyl acetate/hexane) to give 720 mg of isobutyl 3-(4-chlorophenyl)-2-isobutoxyacrylate as an oil. ¹H NMR (400 MHz, CDCl₃) δ ppm 1.00 (12 H, m), 2.05 (2H, m), 3.69 (2H, d), 4.03 (2H, d), 6.89 (1H, s), 7.33 (2H, d), 7.70 (2H, d); HPLC retention time: 6.30 min. (Method D).

### Step 3 Preparation of isobutyl 3-(4-chlorophenyl)-2-isobutoxypropanoate

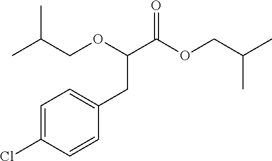 [SMILES: CC(C)COC(=O)C(Cc1ccc(Cl)cc1)OCC(C)C]

To a well-stirred slurry of isobutyl (2Z)-3-(4-chlorophenyl)-2-isobutoxyacrylate (0.720 g, 2.32 mmol) and zinc dibromide (0.104 g, 0.463 mmol) in ethyl acetate (20 mL, 200 mmol) is added 10% palladium on carbon (0.039 g, 0.37 mmol). The reaction mixture is charged with 1 atm of hydrogen gas (balloon; evacuate/charge 5×) and stirred at rt for 24 h. The hydrogen gas is replaced and stirring is continued for an additional 18 h. The reaction mixture is filtered through Celite, the filter pad is washed with ethyl acetate (4×25 mL) and the filtrates are combined. The solution is washed with saturated, aqueous sodium bicarbonate, water and brine and dried with anhydrous sodium sulfate. Concentration provided 720 mg of isobutyl 3-(4-chlorophenyl)-2-isobutoxypropanoate as a clear colorless oil. ¹H NMR (400 MHz, CDCl₃) δ ppm 0.86 (6H, d), 0.93 (6H, d), 1.83 (1H, m), 1.95 (1H, m), 3.01 (3H, m), 3.40 (1H, dd), 3.91 (2H, m), 3.99 (1H, m), 7.21 (2H, d), 7.29 (2H, d); HPLC retention time: 5.94 min. (Method D).

### Step 4 Preparation of 3-(4-chlorophenyl)-2-isobutoxypropan-1-ol

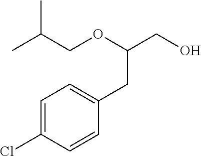 [SMILES: CC(C)COC(CO)Cc1ccc(Cl)cc1]

A slurry of LAH (0.175 g, 4.60 mmol) in dry THF (20 mL) is stirred at 0° C. under nitrogen and a solution of isobutyl 3-(4-chlorophenyl)-2-isobutoxypropanoate (0.72 g, 2.3 mmol) in dry THF (20 mL) is added slowly. The reaction is allowed to warm to rt and is stirred overnight. The reaction mixture is cooled at 0° C. and sodium sulfate decahydrate (200 mg) is added carefully. The mixture is then stirred at rt for 4 h. Water (2.0 mL) is added, the mixture is diluted with ether and filtered through Celite. The salts are washed with ether and the filtrate concentrated. The residue is subjected to silica gel chromatography (230-400 mesh, 50 g, elution with 10 and 20% ethyl acetate/hexane) to give 394 mg of 3-(4-chlorophenyl)-2-isobutoxypropan-1-ol as a clear colorless oil. ¹H NMR (400 MHz, CDCl₃) δ ppm 0.88 (6H, m), 1.81 (1H, m), 1.92 (1H, t), 2.73 (1H, m), 2.86 (1H, m), 3.29 (2H), 3.48 (2H, m), 3.64 (1H, m), 7.14 (2H, d), 7.25 (2H, d); MS (ESI+) for C₁₃H₁₉ClO₂ m/z 265.1 (M+Na)+; HPLC retention time: 4.45 min. (Method D).

### Step 5 Preparation of 1-(3-bromo-2-isobutoxypropyl)-4-chlorobenzene

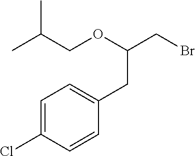 [SMILES: CC(C)COC(CBr)Cc1ccc(Cl)cc1]

A solution of triphenylphosphine (460 mg, 1.8 mmol) in DCM (20 mL) is cooled at 0° C. and a solution of bromine (0.091 mL, 1.8 mmol) in DCM (10 mL) added slowly over a period of 30 min. A solution of 3-(4-chlorophenyl)-2-isobutoxypropan-1-ol (390 mg, 1.6 mmol) in DCM (10 mL) is then added and the reaction mixture is allowed to warm to rt and is stirred for 24 h. The reaction mixture is then transferred to a separatory funnel; washed with saturated, aqueous sodium bicarbonate, water and brine. The organic layer is dried with anhydrous sodium sulfate and concentrated. The residue is subjected to silica gel chromatography (230-400 mesh, 350 g, elution with 5% ethyl acetate/hexane) to give 470 mg of 1-(3-bromo-2-isobutoxypropyl)-4-chlorobenzene as a clear colorless oil. ¹H NMR (400 MHz, CDCl₃) δ ppm 0.87 (6H, m), 1.80 (1H, m), 2.86 (1H, dd), 2.96 (1H, dd), 3.11 (1H, dd), 3.31 (1H, dd), 3.37 (2H, m), 3.60 (1H, m), 7.20 (2H, d), 7.28 (2H, d); HPLC retention time: 5.85 min. (Method D).

### Step 6 Preparation of N-[3-(4-chlorophenyl)-2-isobutoxypropy]-4-isopropyl-2-nitroaniline

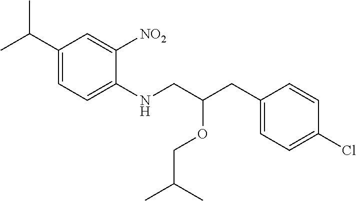 [SMILES: CC(C)COC(CNc1ccc(C(C)C)cc1[N+](=O)[O-])Cc1ccc(Cl)cc1]

To a cold (at 0° C.) solution 4-isopropyl-2-nitroaniline (0.23 g, 1.3 mmol) in dry DMF (7.5 mL) is added sodium hydride (61 mg, 1.53 mmol) portionwise. After 15 min., the cooling bath is removed and the solution is stirred 30 min. at rt. To this solution is added 1-(3-bromo-2-isobutoxypropyl)-4-chlorobenzene (0.468 g, 1.53 mmol) dropwise via syringe. After 18 h at rt, the reaction mixture is heated at 60° C. for 8 h. This mixture is concentrated in vacuo to remove DMF and the residue is partitioned between DCM and saturated, aqueous ammonium chloride (50 mL each). The layers are separated, the aqueous layer is extracted with DCM (3×20 mL), and the combined organic layers are dried with anhydrous sodium sulfate and concentrated. The residue is subjected to silica gel chromatography (230-400 mesh, 150 g, elution with 5% EtOAc/hexane) to give 335 mg of N-[3-(4-chlorophenyl)-2-isobutoxypropyl]-4-isopropyl-2-nitroaniline as an orange oil. ¹H NMR (400 MHz, CDCl₃) δ ppm 0.89 (12H, m), 1.81 (2H, m), 6.67 (1H, d), 7.16 (3H, m), 7.32 (2H, m), 8.02 (1H, d); HPLC retention time: 6.50 min. (Method D).

### Step 7 Preparation of N1-[3-(4-chlorophenyl)-2-isobutoxypropyl]-4-isopropylbenzene-1,2-diamine

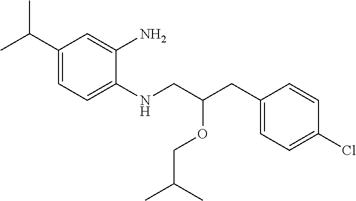 [SMILES: CC(C)COC(CNc1ccc(C(C)C)cc1N)Cc1ccc(Cl)cc1]

A slurry of N-[3-(4-chlorophenyl)-2-isobutoxypropyl]-4-isopropyl-2-nitroaniline (0.335 g, 0.827 mmol) and Raney Nickel (0.200 g, 3.41 mmol) in ethanol (20 mL) is subjected to 1 atm of hydrogen gas (balloon) for 2 h. The reaction mixture is diluted with ethanol (10 mL), and filtered through Celite. The filter pad is washed with ethanol (10 mL) and the filtrates are combined and concentrated. The residue is subjected to silica gel chromatography (230-400 mesh, 50 g, elution with 10, 15 and 20% ethyl acetate/hexane) to give 64 mg of N1-[3-(4-chlorophenyl)-2-isobutoxypropyl]-4-isopropylbenzene-1,2-diamine as a clear colorless oil. ¹H NMR (400 MHz, CDCl₃) δ ppm 0.78 (6H, d), 1.10 (6H, d), 1.67 (1H, m), 2.63 (1H, m), 2.81 (1H, m), 2.90 (1H, m), 2.99 (2H, m), 3.12 (1H, dd), 3.24 (1H, dd), 3.65 (1H, m), 4.10 (1H, t), 4.42 (2H, br s), 6.27 (1H, d), 6.34 (1H, dd), 6.45 (1H, s), 7.27 (2H, d), 7.33 (2H, d); MS (ESI⁺) for C₂₂H₃₁ClN₂O m/z 375.2 (M+H)⁺; HPLC retention time: 4.53 min. (Method D).

### Step 8 Preparation of 10-[3-(4-chlorophenyl)-2-isobutoxypropyl]-7-isopropylbenzo[g]pteridine-2,4(3H,10H)-dione

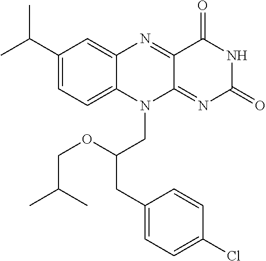 [SMILES: CC(C)COC(Cc1ccc(Cl)cc1)Cn1c2nc(=O)[nH]c(=O)c-2nc2cc(C(C)C)ccc21]

To a well-stirred solution of N1-[3-(4-chlorophenyl)-2-isobutoxypropyl]-4-isopropylbenzene-1,2-diamine (155.0 mg, 0.4134 mmol) and alloxan (72.8 mg, 0.455 mmol) in acetic acid (10 mL) is added boric acid (76.7 mg, 1.24 mmol). The reaction mixture is then stirred at rt for 18 h. The reaction is concentrated in vacuo, suspended in 10% MeOH/DCM (10 mL) and filtered. The yellow solution is concentrated to provide a a solid. This solid is dissolved in DCM, adsorbed onto silica gel (10 g) and subjected to silica gel chromatography (230-400 mesh, 50 g, elution with 0.5, 1, 1.5% MeOH/DCM) to give 132 mg of 10-[3-(4-chlorophenyl)-2-isobutoxypropyl]-7-isopropylbenzo[g]pteridine-2,4(3H,10H)-dione as an oil. ¹H NMR (400 MHz, DMSO-d₆) δ ppm 0.40 (6H, m), 0.85 (1H, m), 1.28 (6H, m), 2.77 (1H, m), 2.96 (3H, m), 3.10 (1H, m), 4.05 (1H, m), 4.67 (2H, br. s), 7.34 (4H, s), 7.84 (1H, dd), 7.91 (2H, m), 11.39 (1 H, s); MS (ESI⁺) for C₂₆H₂₉ClN₄O₃ m/z 481.0 (M+H)⁺; HPLC retention time: 4.91 min. (Method D).

### Example 30

### 10-[2-(Benzyloxy)-3-phenylpropyl]-7,8-dimethylbenzo[g]pteridine-2,4(3H,10H)-dione

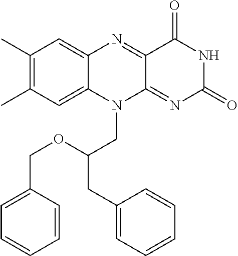 [SMILES: Cc1cc2nc3c(=O)[nH]c(=O)nc-3n(CC(Cc3ccccc3)OCc3ccccc3)c2cc1C]

### Step 1 Preparation of [(2-azido-1-benzylethoxy)methyl]benzene

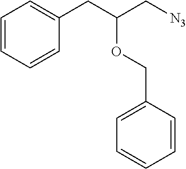 [SMILES: [N-]=[N+]=NCC(Cc1ccccc1)OCc1ccccc1]

To a cold (at 0° C.) solution of 1-azido-3-phenylpropan-2-ol (0.5 g, 3 mmol) in dry THF (21 mL) under nitrogen is added sodium hydride (0.135 g, 3.38 mmol) as a solid. This mixture is stirred an additional 30 min. at 0° C. and benzyl bromide (0.420 mL, 3.53 mmol) added via syringe. The reaction is then allowed to warm to rt and is stirred overnight. The reaction mixture is partitioned between saturated, aqueous ammonium chloride and ethyl acetate (30 mL each), the layers are separated and the aqueous layer is extracted with ethyl acetate (3×20 mL). The organic layers are combined, dried with anhydrous sodium sulfate and concentrated. The residue is subjected to silica gel chromatography (230-400 mesh,40 g, elution with 10% ethyl acetate/hexane) to give 614 mg of [(2-azido-1-benzylethoxy)methyl]benzene as a clear colorless oil. ¹H NMR (400 MHz, CDCl₃) δ ppm 2.83 (1H, m), 2.96 (1H, m), 3.28 (2H, m), 3.77 (1H, m), 4.56 (2H, m), 7.29 (10H, m); HPLC retention time: 4.96 min. (Method D).

### Step 2 Preparation of 2-(Benzyloxy)-3-phenylpropan-1-amine

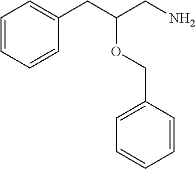 [SMILES: NCC(Cc1ccccc1)OCc1ccccc1]

To a cold (at 0° C.) well-stirred solution of [(2-azido-1-benzylethoxy)methyl]benzene (0.614 g, 2.30 mmol) in dry THF (10 mL) is added a 1.00 M solution of trimethylphosphine in THF (3.44 mL, 3.44 mmol). After 30 min. at 0° C., the ice bath is removed and stirring is continued for 18 h at rt. The reaction mixture is cooled at 0° C. and water (0.5 mL) is added. The reaction mixture is then allowed to warm to rt and is stirred overnight. The reaction mixture is partitioned between brine and ethyl acetate (50 mL each), the layers are separated and the aqueous layer is extracted with ethyl acetate (3×25 mL). The organic layers are combined, dried with anhydrous sodium sulfate and concentrated. The residue is subjected to silica gel chromatography (230-400 mesh, 50 g, elution with 1 and 2% MeOH (containing 7M NH₃)/DCM) to give 275 mg of 2-(benzyloxy)-3-phenylpropan-1-amine as an oil. ¹H NMR (400 MHz, CDCl₃) δ ppm 1.48 (2H, br s), 2.80 (3H, m), 3.00 (1H, dd), 3.65 (1H, m), 4.56 (2H, m), 7.35 (10H, m); MS (ESI⁺) for C₁₆H₁₉NO m/z 242.2 (M+H)⁺; HPLC retention time: 3.02 min. (Method D).

### Step 3 Preparation of N-[2-(benzyloxy)-3-phenylpropyl]-4,5-dimethyl-2-nitroaniline

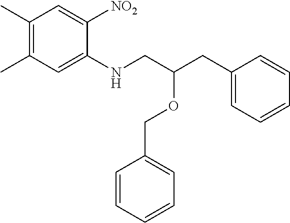 [SMILES: Cc1cc(NCC(Cc2ccccc2)OCc2ccccc2)c([N+](=O)[O-])cc1C]

A well-stirred slurry of 1-bromo-4,5-dimethyl-2-nitrobenzene (0.364 g, 1.58 mmol), 2-(benzyloxy)-3-phenylpropan-1-amine (0.273 g, 1.13 mmol), Cs₂CO₃ (737 mg, 2.26 mmol) and (oxydi-2,1-phenylene)bis[diphenylphosphine] (91.4 mg, 0.170 mmol) in toluene (10 mL) is sparged with dry nitrogen for 5 min. Tris(dibenzylideneacetone)dipalladium(0) (51.8 mg, 0.0566 mmol) is added and sparging is continued for an additional 5 min. The reaction mixture is then heated at 100° C. for 48 h. The mixture is cooled to rt, diluted with ethyl acetate (15 mL), filtered and the salts are washed with ethyl acetate (3×10 mL). The organic layers are combined, concentrated and the residue is subjected to silica gel chromatography (230-400 mesh, 150 g, elution with 10% ethyl acetate/hexane) which gives 198 mg of N-[2-(benzyloxy)-3-phenylpropyl]-4,5-dimethyl-2-nitroaniline as an orange oil. ¹H NMR (400 MHz, CDCl₃) δ ppm 2.16 (6H, s), 2.86 (1H, dd), 3.10 (1H, dd), 3.28 (1H, m), 3.40 (1H, m), 3.89 (1H, m), 4.59 (2H, m), 6.37 (1H, s), 7.30 (10H, m), 7.92 (1H, s), 8.25 (1H, m); MS (ESI⁺) for C₂₄H₂₆N₂O₃ m/z 391.3 (M+H)+; HPLC retention time: 5.57 min. (Method D).

### Step 4 Preparation of N-[2-(benzyloxy)-3-phenylpropyl]-4,5-dimethylbenzene-1,2-diamine

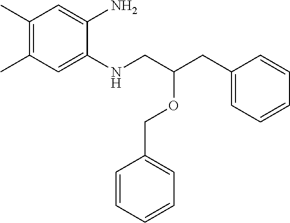 [SMILES: Cc1cc(N)c(NCC(Cc2ccccc2)OCc2ccccc2)cc1C]

To a well-stirred solution of N-[2-(benzyloxy)-3-phenylpropyl]-4,5-dimethyl-2-nitroaniline (155 mg, 0.397 mmol) and solid ammonium chloride (210 mg, 4.0 mmol) in MeOH (20 mL) at 0° C. is added zinc (520 mg, 7.9 mmol) as a solid. After 5 min, the reaction is diluted with ethyl acetate and filtered through Celite. The solution is washed with water, dried with anhydrous sodium sulfate and concentrated to give 140 mg of N-[2-(benzyloxy)-3-phenylpropyl]-4,5-dimethylbenzene-1,2-diamine as an oil. ¹H NMR (400 MHz, CDCl₃) δ ppm 2.13 (6H, s), 2.91 (1H, dd), 3.09 (2H, m), 3.19 (1H, dd), 3.69 (3H, br. s), 4.08 (1H, m), 4.58 (2H, s), 6.51 (1H, s), 6.65 (1H, s), 7.32 (10H, m); MS (ESI⁺) for C₂₄H₂₈N₂O m/z 361.1 (M+H)⁺; HPLC retention time: 4.00 min. (Method D).

### Step 5 Preparation of 10-[2-(benzyloxy)-3-phenylpropyl]-7,8-dimethylbenzo[g]pteridine-2,4(3H,10H)-dione

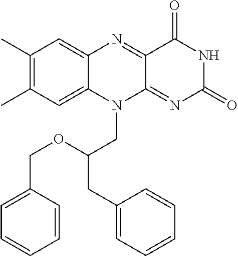 [SMILES: Cc1cc2nc3c(=O)[nH]c(=O)nc-3n(CC(Cc3ccccc3)OCc3ccccc3)c2cc1C]

To a well-stirred solution of N-[2-(benzyloxy)-3-phenylpropyl]-4,5-dimethylbenzene-1,2-diamine (140.0 mg, 0.3884 mmol) and alloxan (68.4 mg, 0.427 mmol) in acetic acid (9 mL) is added boric acid (72.0 mg, 1.16 mmol). The reaction is stirred at rt for 18 h and concentrated in vacuo. The solid is dissolved in 10% MeOH/DCM, filtered, adsorbed onto silica gel (10 g) and subjected to silica gel chromatography (230-400 mesh, 50 g, elution with 0.5, 1, 1.5% MeOH/DCM) to give 121 mg of 10-[2-(benzyloxy)-3-phenylpropyl]-7,8-dimethylbenzo[g]pteridine-2,4(3H,10H)-dione as a orange-red solid. ¹H NMR (400 MHz, DMSO-d₆) δ ppm 2.36 (3H, s), 2.38 (3H, s), 3.09 (2H, d), 4.02 (1H, m), 4.24 (2 H, m), 4.58 (1H, m), 4.70 (1H, m), 6.62 (2H, d), 6.96 (2H, t), 7.05 (1H, m), 7.26 (1H, t), 7.35 (4H, m), 7.57 (1H, br s), 7.85 (1H, s), 11.26 (1H, s); MS (ESI⁺) for C₂₈H₂₆N₄O₃ m/z 467.2 (M+H)⁺; HPLC retention time: 4.02 min. (Method D).

The compounds of the invention particularly those compounds as set forth in Table 1 below which are disclosed and claimed either individually and/or collectively may generally be prepared using similar procedures as set forth in Examples 1-16 above. It is to be understood that the appropriate reagents, solvents and reaction condition for those reactions are used as apparent to one skilled in the art.

TABLE 1

| | | | HPLC | | | |
|---|---|---|---|---|---|---|
| | | LC-MS | retention | HPLC | | |
| | | MH+ | time | meth- | | |
| Entry | Structure | (m/z) | (min) | od | Preparation | Name |
| 1 | 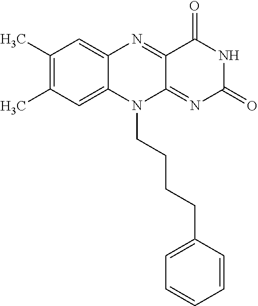 [SMILES: Cc1cc2nc3c(=O)[nH]c(=O)nc-3n(CCCCc3ccccc3)c2cc1C] | 375.1 | 5.68 | Meth- od F | Prepared using synthesis of Example 1 starting from 4,5-dimethyl-2- nitroaniline and (4- bromobutyl)benz- ene | 7,8- dimethyl- 10-(4- phenylbutyl) benzo[g] pteridine- 2,4(3H,10H)- dione |
| 2 | 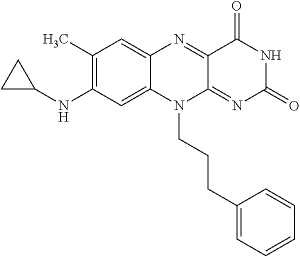 [SMILES: Cc1cc2nc3c(=O)[nH]c(=O)nc-3n(CCCc3ccccc3)c2cc1NC1CC1] | 402.2 | 5.15 | Meth- od F | Prepared using synthesis of Example 10 starting from 8- chloro-7-methyl- 10-(3- phenylpropyl) benzo[g]pteridine- 2,4(3H,10H)- dione and cyclopropylamine | 8- (cyclopropyl- amino)-7- methyl 10- (3- phenylprop- yl)benzo[g] pteridine- 2,4(3H,10H)- dione |
| 3 | 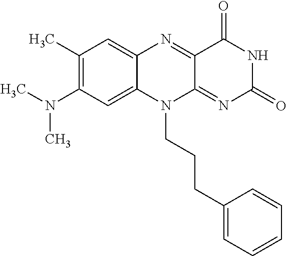 [SMILES: Cc1cc2nc3c(=O)[nH]c(=O)nc-3n(CCCc3ccccc3)c2cc1N(C)C] | 390.2 | 5.24 | Meth- od F | Prepared using synthesis of Example 10 starting from 8- chloro-7-methyl- 10-(3- phenylpropyl) benzo[g]pteridine- 2,4(3H,10H)- dione and dimethylamine | 8- (dimethyl- amino)-7- methyl-10- (3- phenylprop- yl)benzo[g] pteridine- 2,4(3H,10H)- dione |
| 4 | 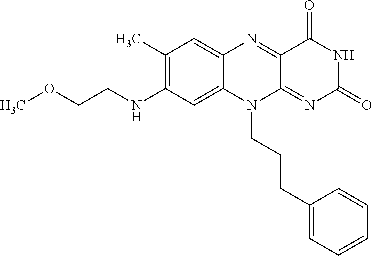 [SMILES: COCCNc1cc2c(cc1C)nc1c(=O)[nH]c(=O)nc-1n2CCCc1ccccc1] | 420.3 | 4.87 | Meth- od F | Prepared using synthesis of Example 10 starting from 8- chloro-7-methyl- 10-(3- phenylpropyl) benzo[g]pteridine- 2,4(3H,10H)- dione and 2- methoxyethyl- amine | 8-[(2- methoxy- ethyl)amino]- 7-methyl- 10-(3- phenylprop- yl) benzo[g] pteridine- 2,4(3H,10H)- dione |
| 5 | 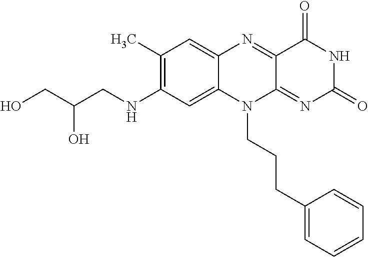 [SMILES: Cc1cc2nc3c(=O)[nH]c(=O)nc-3n(CCCc3ccccc3)c2cc1NCC(O)CO] | 436.2 | 4.02 | Meth- od F | Prepared using synthesis of Example 10 starting from 8- chloro-7-methyl- 10-(3- phenylpropyl) benzo[g]pteridine- 2,4(3H,10H)- dione and 3- amino-1,2- propanediol | 8-[(2,3- dihydroxy- propyl)amino]- 7- methyl-10- (3- phenylprop- yl) benzo[g] pteridine- 2,4(3H,10H)- dione |
| 6 | 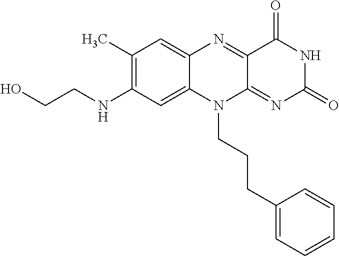 [SMILES: Cc1cc2nc3c(=O)[nH]c(=O)nc-3n(CCCc3ccccc3)c2cc1NCCO] | 406.3 | 4.27 | Meth- od F | Prepared using synthesis of Example 10 starting from 8- chloro-7-methyl- 10-(3- phenylpropyl) benzo[g]pteridine- 2,4(3H,10H)- dione and ethanolamine | 8-[(2- hydroxyeth- yl)amino]- 7-methyl- 10-(3- phenylprop- yl) benzo[g] pteridine- 2,4(3H,10H)- dione |
| 7 | 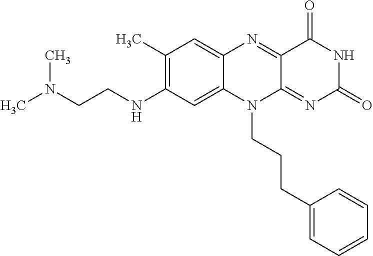 [SMILES: Cc1cc2nc3c(=O)[nH]c(=O)nc-3n(CCCc3ccccc3)c2cc1NCCN(C)C] | 433.3 | 3.88 | Meth- od F | Prepared using synthesis of Example 10 starting from 8- chloro-7-methyl- 10-(3- phenylpropyl) benzo[g]pteridine- 2,4(3H,10H)- dione and N,N- dimethyl-1,2- ethanediamine | 8-{[2- (dimethyl- amino)ethyl] amino}-7- methyl-10- (3- phenylprop- yl) benzo[g] pteridine- 2,4(3H,10H)- dione |
| 8 | 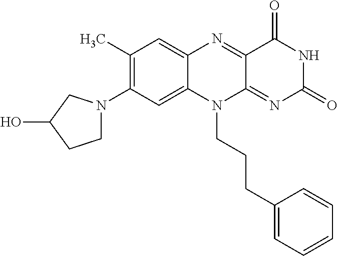 [SMILES: Cc1cc2nc3c(=O)[nH]c(=O)nc-3n(CCCc3ccccc3)c2cc1N1CCC(O)C1] | 432.2 | 2.94 | Meth- od G | Prepared using synthesis of Example 10 starting from 8- chloro-7-methyl- 10-(3- phenylpropyl) benzo[g]pteridine- 2,4(3H,10H)- dione and 3- pyrrolidinol | 8-(3- hydroxy- pyrrolidin-1- yl)-7- methyl-10- (3- phenylprop- yl) benzo[g] pteridine- 2,4(3H,10H)- dione |
| 9 | 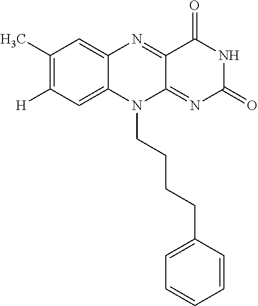 [SMILES: Cc1ccc2c(c1)nc1c(=O)[nH]c(=O)nc-1n2CCCCc1ccccc1] | 361.2 | 3.91 | Meth- od G | Prepared using synthesis of Example 13 starting from 8- chloro-7-methyl- 10-(4- phenylbutyl)benzo [g]pteridine- 2,4(3H,10H)- dione | 7-methyl- 10-(4- phenylbutyl) benzo[g] pteridine- 2,4(3H,10H)- dione |
| 10 | 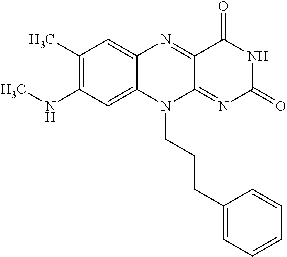 [SMILES: CNc1cc2c(cc1C)nc1c(=O)[nH]c(=O)nc-1n2CCCc1ccccc1] | 376.2 | 3.14 | Meth- od G | Prepared using synthesis of Example 10 starting from 8- chloro-7-methyl- 10-(3- phenylpropyl) benzo[g]pteridine- 2,4(3H,10H)- dione and methylamine | 7-methyl- 8- (methyl- amino)-10-(3- phenylprop- yl)benzo[g] pteridine- 2,4(3H,10H)- dione |
| 11 | 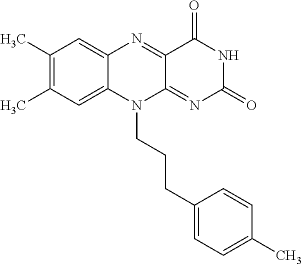 | 375.2 | 4.09 | Meth- od G | Prepared using synthesis of Example 1 starting from 4,5-dimethyl-2- nitroaniline and 1-(3- bromopropyl)-4- methylbenzene | 7,8- dimethyl- 10-[3-(4- methylphen- yl)propyl] benzo[g] pteridine- 2,4(3H,10H)- dione |
| 12 | 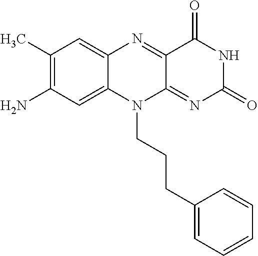 [SMILES: Cc1cc2nc3c(=O)[nH]c(=O)nc-3n(CCCc3ccccc3)c2cc1N] | 362.3 | 2.90 | Meth- od G | Prepared using synthesis of Example 10 starting from 8- chloro-7-methyl- 10-(3- phenylpropyl) benzo[g]pteridine- 2,4(3H,10H)- dione and ammonium hydroxide | 8-amino-7- methyl-10- (3- phenylprop- yl)benzo[g] pteridine- 2,4(3H,10H)- dione |
| 13 | 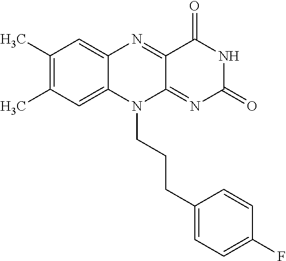 [SMILES: Cc1cc2nc3c(=O)[nH]c(=O)nc-3n(CCCc3ccc(F)cc3)c2cc1C] | 379.1 | 3.55 | Meth- od D | Prepared using synthesis of Example 6 starting from 1- bromo-4,5- dimethyl-2- nitrobenzene and 3-(4- fluorophenyl) propan-1-amine | 10-[3-(4- fluorophen- yl)propyl]- 7,8- dimethyl- benzo[g] pteridine- 2,4(3H,10H)- dione |
| 14 | 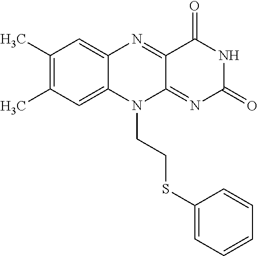 [SMILES: Cc1cc2nc3c(=O)[nH]c(=O)nc-3n(CCSc3ccccc3)c2cc1C] | 379.1 | 3.41 | Meth- od D | Prepared using synthesis of Example 4 starting from 1- bromo-4,5- dimethyl-2- nitrobenzene and 1- phenylthio-2- bromoethane | 7,8- dimethyl- 10-[2- (phenylthio) ethyl]benzo [g]pteridine- 2,4(3H,10H)- dione |
| 15 | 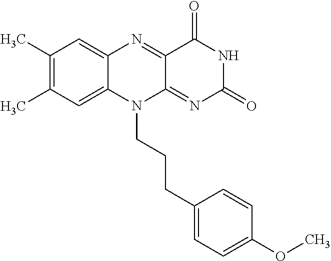 | 391.4 | 6.38 | Meth- od C | Prepared using synthesis of Example 1 starting from 4,5-dimethyl-2- nitroaniline and 1-(3- bromopropyl)- 4- methoxybenzene | 10-[3-(4- methoxy- phenyl)propyl]- 7,8- dimethyl- benzo[g] pteridine- 2,4(3H,10H)- dione |
| 16 | 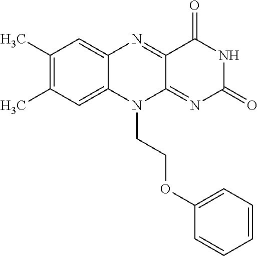 [SMILES: Cc1cc2nc3c(=O)[nH]c(=O)nc-3n(CCOc3ccccc3)c2cc1C] | 362.4 | 4.22 | Meth- od A | Prepared using synthesis of Example 1 starting from 4,5-dimethyl-2- nitroaniline and (2- bromoethoxy)- benzene | 7,8- dimethyl- 10-(2- phenoxy- ethyl)benzo [g]pteridine- 2,4(3H,10H)- dione |
| 17 | 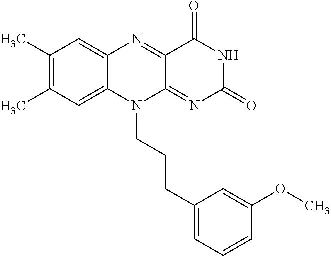 | 390.4 | 4.23 | Meth- od A | Prepared using synthesis of Example 1 starting from 4,5-dimethyl-2- nitroaniline and 1-(3- bromopropyl)- 3- methoxybenzene | 10-[3-(3- methoxy- phenyl)propyl]- 7,8- dimethyl- benzo[g] pteridine- 2,4(3H,10H)- dione |
| 18 | 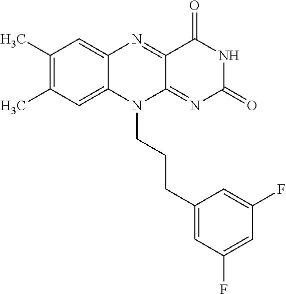 [SMILES: Cc1cc2nc3c(=O)[nH]c(=O)nc-3n(CCCc3cc(F)cc(F)c3)c2cc1C] | 396.1 | 4.47 | Meth- od E | Prepared using synthesis of Example 1 starting from 4,5-dimethyl-2- nitroaniline and 1-(3- bromopropyl)- 3,5- difluorobenzene | 10-[3-(3,5- difluoro- phenyl)propyl]- 7,8- dimethyl- benzo[g] pteridine- 2,4(3H,10H)- dione |
| 19 | 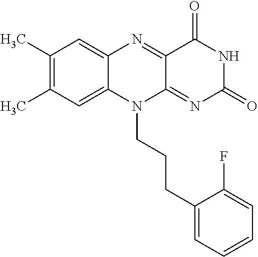 [SMILES: Cc1cc2nc3c(=O)[nH]c(=O)nc-3n(CCCc3ccccc3F)c2cc1C] | 378.4 | 4.39 | Meth- od E | Prepared using synthesis of Example 1 starting from 4,5-dimethyl-2- nitroaniline and 1-(3- bromopropyl)- 2- fluorobenzene | 10-[3-(2- fluorophen- yl)propyl]- 7,8- dimethyl- benzo[g] pteridine- 2,4(3H,10H)- dione |
| 21 | 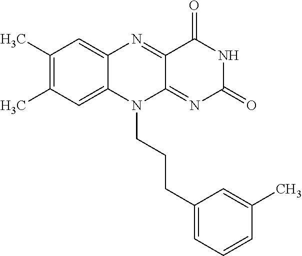 | 374.4 | 3.76 | Meth- od E | Prepared using synthesis of Example 1 starting from 4,5-dimethyl-2- nitroaniline and 3-(3- methylphenyl) propylbromide | 7,8- Dimethyl- 10-[3-(3- methyl- phenyl)propyl] benzo[g] pteridine- 2,4(3H,10H)- dione |
| 22 | 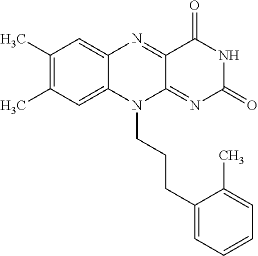 [SMILES: Cc1cc2nc3c(=O)[nH]c(=O)nc-3n(CCCc3ccccc3C)c2cc1C] | 374.4 | 3.75 | Meth- od E | Prepared using synthesis of Example 1 starting from 4,5-dimethyl-2- nitroaniline and 3-(2- methylphenyl) propylbromide | 7,8- Dimethyl- 10-[3-(2- methyl- phenyl)propyl] benzo[g] pteridine- 2,4(3H,10H)- dione |
| 23 | 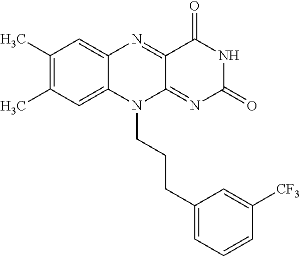 [SMILES: Cc1cc2nc3c(=O)[nH]c(=O)nc-3n(CCCc3cccc(C(F)(F)F)c3)c2cc1C] | 428.1 | 3.97 | Meth- od E | Prepared using synthesis of Example 4 starting from 4,5-Dimethyl- o- phenylenedi- amine and 1-(3- bromopropyl)- 3- (trifluoromethyl) benzene | 7,8- dimethyl- 10-{3-[3- (trifluoro- methyl)phen- yl]propyl} benzo[g] pteridine- 2,4(3H,10H)- dione |
| 24 | 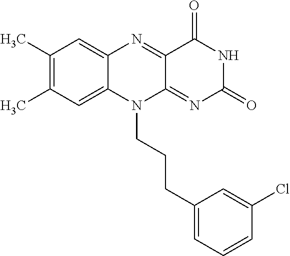 [SMILES: Cc1cc2nc3c(=O)[nH]c(=O)nc-3n(CCCc3cccc(Cl)c3)c2cc1C] | 394.8 | 5.57 | Meth- od E | Prepared using synthesis of Example 1 starting from 4,5-dimethyl-2- nitroaniline and 1-(3- bromopropyl)- 3- chlorobenzene | 10-[3-(3- chlorophen- yl)propyl]- 7,8- dimethyl- benzo[g] pteridine- 2,4(3H,10H)- dione |
| 25 | 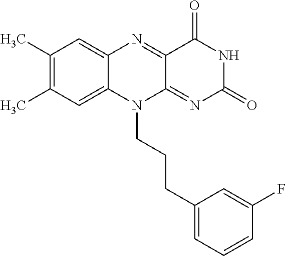 [SMILES: Cc1cc2nc3c(=O)[nH]c(=O)nc-3n(CCCc3cccc(F)c3)c2cc1C] | 378.4 | 4.39 | Meth- od D | Prepared using synthesis of Example 1 starting from 4,5-dimethyl-2- nitroaniline and 1-(3- bromopropyl)- 3- fluorobenzene | 10-[3-(3- fluorophen- yl)propyl]- 7,8- dimethyl- benzo[g] pteridine- 2,4(3H,10H)- dione |
| 26 | 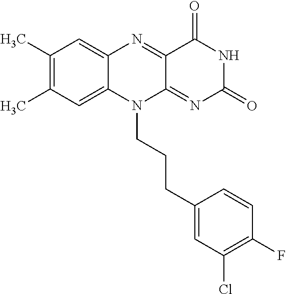 [SMILES: Cc1cc2nc3c(=O)[nH]c(=O)nc-3n(CCCc3ccc(F)c(Cl)c3)c2cc1C] | 412.8 | 3.82 | Meth- od D | Prepared using synthesis of Example 1 starting from 4,5-dimethyl-2- nitroaniline and 4-(3- bromopropyl)- 2-chloro-1- fluorobenzene | 10-[3-(3- chloro-4- fluorophen- yl)propyl]- 7,8- dimethyl- benzo[g] pteridine- 2,4(3H,10H)- dione |
| 27 | 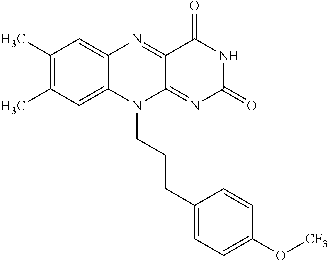 [SMILES: Cc1cc2nc3c(=O)[nH]c(=O)nc-3n(CCCc3ccc(OC(F)(F)F)cc3)c2cc1C] | 444.4 | 4.06 | Meth- od E | Prepared using synthesis of Example 4 starting from 4,5-Dimethyl-o- phenylenediamine and 1-(3- bromopropyl)-4- (trifluoromethoxy) benzene | 7,8- dimethyl- 10-{3-[4- (trifluoro- methoxy)phen- yl]propyl} benzo[g] pteridine- 2,4(3H,10H)- dione |
| 28 | 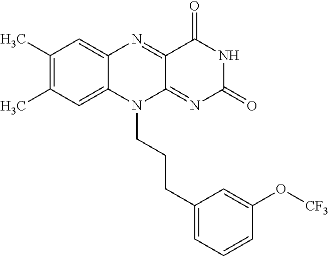 [SMILES: Cc1cc2nc3c(=O)[nH]c(=O)nc-3n(CCCc3cccc(OC(F)(F)F)c3)c2cc1C] | 444.4 | 4.03 | Meth- od E | Prepared using synthesis of Example 4 starting from 4,5-Dimethyl-o- phenylenediamine and 1-(3- bromopropyl)-3- (trifluoromethoxy) benzene | 7,8- dimethyl- 10-{3-[3- (trifluoro- methoxy)phen- yl]propyl} benzo[g] pteridine- 2,4(3H,10H)- dione |
| 29 | 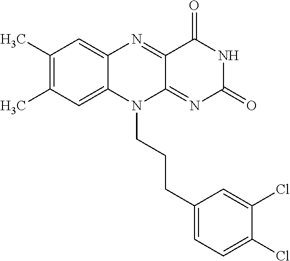 [SMILES: Cc1cc2nc3c(=O)[nH]c(=O)nc-3n(CCCc3ccc(Cl)c(Cl)c3)c2cc1C] | 429.3 | 4.04 | Meth- od E | Prepared using synthesis of Example 4 starting from alkylation of 4,5-Dimethyl-o- phenylenediamine and 1-(3- bromopropyl)- 3,4- dichlorobenzene | 10-[3-(3,4- dichloro- phenyl)propyl]- 7,8- dimethyl- benzo[g] pteridine- 2,4(3H,10H)- dione |
| 30 | 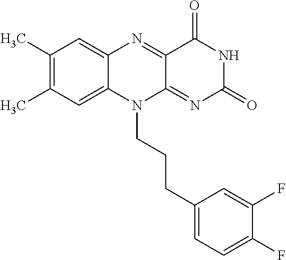 [SMILES: Cc1cc2nc3c(=O)[nH]c(=O)nc-3n(CCCc3ccc(F)c(F)c3)c2cc1C] | 396.4 | 3.67 | Meth- od E | Prepared using synthesis of Example 4 starting from 4,5-Dimethyl-o- phenylenediamine and 1-(3- bromopropyl)- 3,4- difluorobenzene | 10-[3-(3,4- difluoro- phenyl)propyl]- 7,8- dimethyl- benzo[g] pteridine- 2,4(3H,10H)- dione |
| 31 | 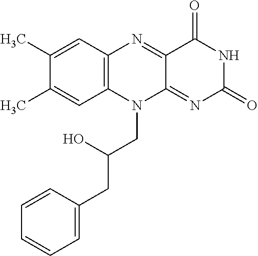 [SMILES: Cc1cc2nc3c(=O)[nH]c(=O)nc-3n(CC(O)Cc3ccccc3)c2cc1C] | 377.1 | 3.29 | C | Prepared using the synthesis of Example 14 | 10-(2- hydroxy-3- phenylprop- yl)-7,8- dimethyl- benzo[g] pteridine- 2,4(3H,10H)- dione |
| 32 | 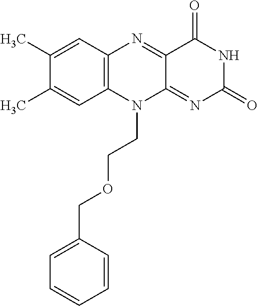 [SMILES: Cc1cc2nc3c(=O)[nH]c(=O)nc-3n(CCOCc3ccccc3)c2cc1C] | 377.3 | 2.53 | C | Prepared using the synthesis of Example 14 | 10-(2- (Benzyloxy) ethyl)-7,8- dimethyl- benzo[g] pteridine- 2,4(3H,10H)- dione |
| 33 | 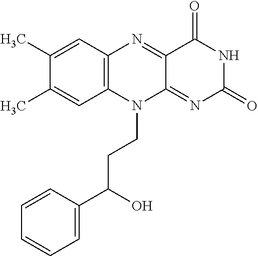 [SMILES: Cc1cc2nc3c(=O)[nH]c(=O)nc-3n(CCC(O)c3ccccc3)c2cc1C] | 377.1 | 2.31 | C | Prepared using the synthesis of Example 14 | 10-(3- hydroxy-3- phenylprop- yl)-7,8- dimethyl- benzo[g] pteridine- 2,4(3H,10H)- dione |
| 34 | 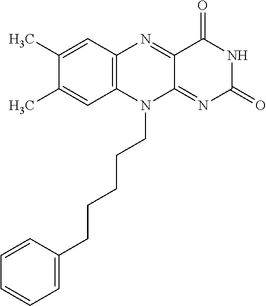 [SMILES: Cc1cc2nc3c(=O)[nH]c(=O)nc-3n(CCCCCc3ccccc3)c2cc1C] | 389.2 | 5.85 | C | Prepared using the synthesis of Example 14 | 7,8- dimethyl- 10-(5- phenylpent- yl)benzo[g] pteridine- 2,4(3H,10H)- dione |
| 35 | 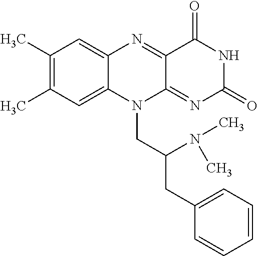 [SMILES: Cc1cc2nc3c(=O)[nH]c(=O)nc-3n(CC(Cc3ccccc3)N(C)C)c2cc1C] | 404.1 | 4.89 | C | Prepared using the synthesis of Example 14 | 10-(2- (dimethyl- amino)-3- phenylprop- yl)-7,8- dimethyl- benzo[g] pteridine 2,4(3H,10H)- dione |
| 36 | 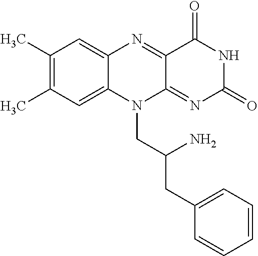 [SMILES: Cc1cc2nc3c(=O)[nH]c(=O)nc-3n(CC(N)Cc3ccccc3)c2cc1C] | 376.1 | 5.69 | C | Prepared using the synthesis of Example 14 | 10-(2- amino-3- phenylprop- yl)-7,8- dimethyl- benzo[g] pteridine- 2,4(3H,10H)- dione |

The compounds of the invention particularly those compounds as set forth in Table 2 below which are disclosed and claimed either individually and/or collectively may generally be prepared using similar procedures as set forth in Examples 1-30 above. It is to be understood that the appropriate reagents, solvents and reaction condition for those reactions are used as apparent to one skilled in the art.

TABLE 2

| | | | HPLC | | | |
|---|---|---|---|---|---|---|
| | | LC- | re- | | | |
| | | MS | tention | HPLC | | |
| | | MH+ | time | meth- | | |
| Entry | Structure | (m/z) | (min) | od | Preparation | Name |
| 1 | 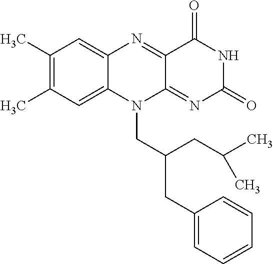 [SMILES: Cc1cc2nc3c(=O)[nH]c(=O)nc-3n(CC(Cc3ccccc3)CC(C)C)c2cc1C] | 417.0 | 4.30 | D | Prepared using the synthesis of Example 6 starting with 1-bromo- 4,5-dimethyl-2- nitrobenzene and 2- benzyl-4- methylpentan-1- amine | 10-(2-benzyl- 4- methylpentyl)- 7,8- dimethylbenzo [g]pteridine- 2,4(3H,10H)- dione |
| 2 | 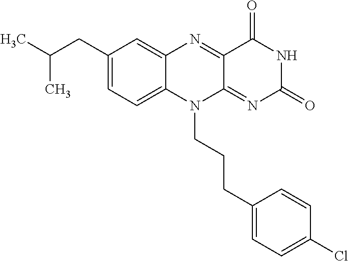 [SMILES: CC(C)Cc1ccc2c(c1)nc1c(=O)[nH]c(=O)nc-1n2CCCc1ccc(Cl)cc1] | 423.0 | 4.37 | D | Prepared using the synthesis of Example 1 starting with 4- isobutyl-2-nitroaniline and 1-(3- bromopropyl)-4- chlorobenzene | 10-(3-(4- chlorophenyl) propyl)-7- isobutylbenzo [g]pteridine- 2,4(3H,10H)- dione |
| 3 | 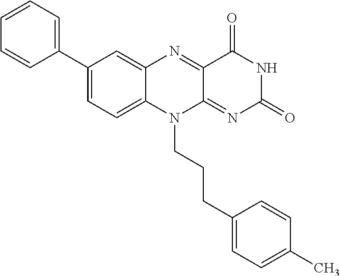 [SMILES: Cc1ccc(CCCn2c3nc(=O)[nH]c(=O)c-3nc3cc(-c4ccccc4)ccc32)cc1] | 423.2 | 4.58 | G | Prepared using the synthesis of Example 1 starting with 1-(3- bromopropyl)-4- methylbenzene and 3- nitrobiphenyl-4-amine | 7-phenyl-10- (3-(p- tolyl)propyl) benzo[g] pteridine- 2,4(3H,10H)- dione |
| 4 | 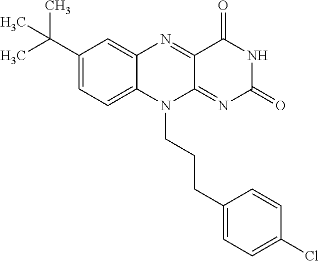 [SMILES: CC(C)(C)c1ccc2c(c1)nc1c(=O)[nH]c(=O)nc-1n2CCCc1ccc(Cl)cc1] | 423.1 | 4.30 | D | Prepared using the synthesis of example 1 using 4-tert-butyl-2- nitroaniline and intermediate B | 7-(tert- butyl)-10-(3- (4- chlorophenyl) propyl)benzo [g]pteridine- 2,4(3H,10H)- dione |
| 5 | 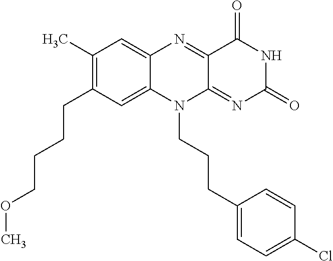 [SMILES: COCCCCc1cc2c(cc1C)nc1c(=O)[nH]c(=O)nc-1n2CCCc1ccc(Cl)cc1] | 467.1 | 4.42 | G | Prepared using the synthesis of Example 1 starting with N-[3-(4- chlorophenyl)propyl]- 5-(4-methoxybutyl)-4- methylbenzene-1,2- diamine, prepared as described for intermediate N. | 10-(3-(4- chlorophenyl) propyl)-8-(4- methoxybutyl)- 7- methylbenzo [g]pteridine- 2,4(3H,10H)- dione |
| 6 | 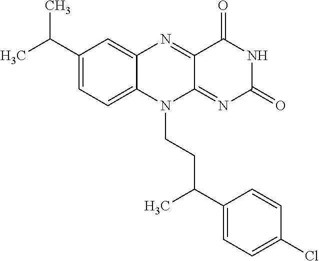 [SMILES: CC(C)c1ccc2c(c1)nc1c(=O)[nH]c(=O)nc-1n2CCC(C)c1ccc(Cl)cc1] | 423.2 | 4.28 | D | Prepared using the synthesis of example 1 starting with 4- isopropyl-2- nitroaniline and intermediate M | 10-(3-(4- chlorophenyl) butyl)-7- isopropylbenzo [g]pteridine- 2,4(3H,10H)- dione |
| 7 | 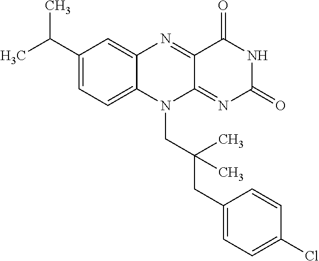 [SMILES: CC(C)c1ccc2c(c1)nc1c(=O)[nH]c(=O)nc-1n2CC(C)(C)Cc1ccc(Cl)cc1] | 437.2 | 4.87 | G | Prepared using the synthesis of Example 17 starting with 3-(4- Chlorophenyl)-2,2- dimethylpropan-1- amine, intermediate O | 10-(3-(4- chlorophenyl)- 2,2- dimethylprop- yl)-7- isopropylbenzo [g]pteridine- 2,4(3H,10H)- dione |
| 8 | 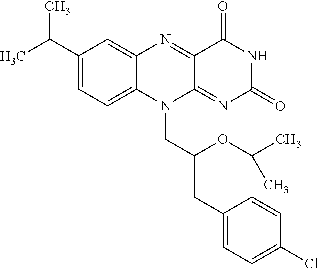 [SMILES: CC(C)OC(Cc1ccc(Cl)cc1)Cn1c2nc(=O)[nH]c(=O)c-2nc2cc(C(C)C)ccc21] | 467.3 | 4.43 | D | Prepared using synthesis of Example 17 starting from 2- bromo-5- isopropylnitrobenzene and 3-(4- chlorophenyl)-2- isopropoxypropan-1- amine | 10-(3-(4- chlorophenyl)- 2- isopropoxy- propyl)-7- isopropylbenzo [g]pteridine- 2,4(3H,10H)- dione |
| 9 | 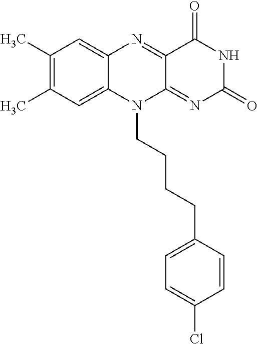 [SMILES: Cc1cc2nc3c(=O)[nH]c(=O)nc-3n(CCCCc3ccc(Cl)cc3)c2cc1C] | 409.1 | 4.37 | G | Prepared using the synthesis of Example 1 starting with 4,5- dimethyl-2- nitroaniline and 1-(4- bromobutyl)-4- chlorobenzene | 10-(4-(4- chlorophenyl) butyl)-7,8- dimethylbenzo [g]pteridine- 2,4(3H,10H)- dione |
| 10 | 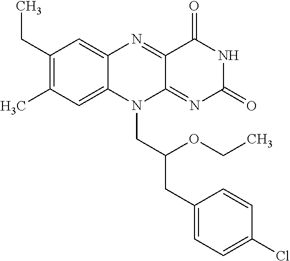 [SMILES: CCOC(Cc1ccc(Cl)cc1)Cn1c2nc(=O)[nH]c(=O)c-2nc2cc(CC)c(C)cc21] | 453.3 | 4.21 | D | Prepared using synthesis of Example 17 starting from 2- bromo-5-ethyl-4- methylnitrobenzene and 3-(4- chlorophenyl)-2- ethoxypropan-1- amine | 10-(3-(4- chlorophenyl)- 2- ethoxypropyl)- 7-ethyl-8- methylbenzo [g]pteridine- 2,4(3H,10H)- dione |
| 11 | 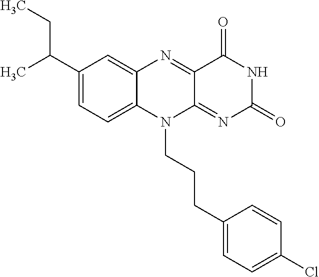 [SMILES: CCC(C)c1ccc2c(c1)nc1c(=O)[nH]c(=O)nc-1n2CCCc1ccc(Cl)cc1] | 423.0 | 4.36 | D | Prepared using the synthesis of Example 1 starting with 4-sec- butyl-2-nitroaniline and 1-(3- bromopropyl)-4- chlorobenzene | 7-(sec-butyl)- 10-(3-(4- chlorophenyl) propyl)benzo [g]pteridine- 2,4(3H,10H)- dione |
| 12 | 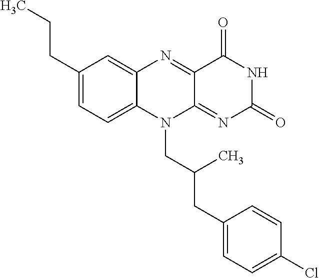 [SMILES: CCCc1ccc2c(c1)nc1c(=O)[nH]c(=O)nc-1n2CC(C)Cc1ccc(Cl)cc1] | 423.2 | 4.29 | D | Prepared using the synthesis of Example 1 starting with N-[3-(4- chlorophenyl)-2- methylpropyl]-2-nitro- 4-propylaniline prepared using the synthesis of intermediate R | 10-(3-(4- chlorophenyl)- 2- methylpropyl)- 7- propylbenzo [g]pteridine- 2,4(3H,10H)- dione |
| 13 | 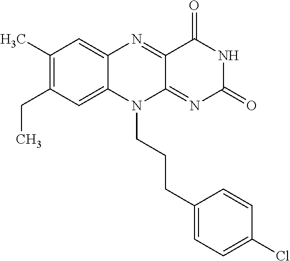 [SMILES: CCc1cc2c(cc1C)nc1c(=O)[nH]c(=O)nc-1n2CCCc1ccc(Cl)cc1] | 423.0 | 4.36 | D | Prepared using the synthesis of Example 1 starting with 5-ethyl- 4-methyl-2- nitroaniline and 1-(3- bromopropyl)-4- chlorobenzene | 10-(3-(4- chlorophenyl) propyl)-8- ethyl-7- methylbenzo [g]pteridine- 2,4(3H,10H)- dione |
| 14 | 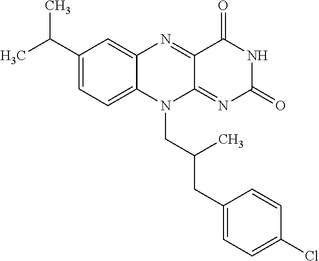 [SMILES: CC(Cc1ccc(Cl)cc1)Cn1c2nc(=O)[nH]c(=O)c-2nc2cc(C(C)C)ccc21] | 423.2 | 4.24 | D | Prepared using the synthesis of Example 1 starting with 4- isopropyp-2- nitroaniline and 1-(3- bromo-2- methylpropyl)-4- chlorobenzene | 10-(3-(4- chlorophenyl)- 2- methylpropyl)- 7- isopropylbenzo [g]pteridine- 2,4(3H,10H)- dione |
| 15 | 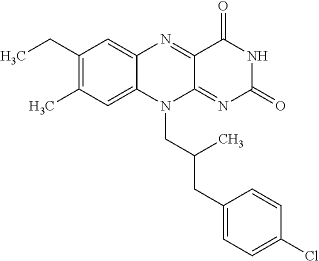 [SMILES: CCc1cc2nc3c(=O)[nH]c(=O)nc-3n(CC(C)Cc3ccc(Cl)cc3)c2cc1C] | 423.2 | 4.19 | D | Prepared using the synthesis of Example 1 starting with N-[3-(4- chlorophenyl)-2- methylpropyl]-2-nitro- 4-propylaniline prepared using the synthesis of intermediate R | 10-(3-(4- chlorophenyl)- 2- methylpropyl)- 7-ethyl-8- methylbenzo [g]pteridine- 2,4(3H,10H)- dione |
| 16 | 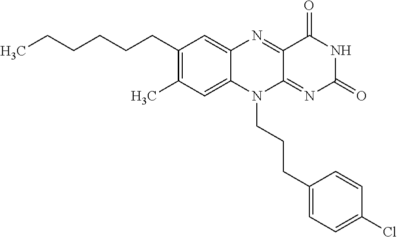 [SMILES: CCCCCCc1cc2nc3c(=O)[nH]c(=O)nc-3n(CCCc3ccc(Cl)cc3)c2cc1C] | 465.3 | 5.61 | G | Prepared using synthesis of Example 1 starting from 4-hexyl- 5-methyl-2- nitroaniline and 1-(3- bromopropyl)-4- chlorobenzene | 10-(3-(4- chlorophenyl) propyl)-7- hexyl-8- methylbenzo [g]pteridine- 2,4(3H,10H)- dione |
| 17 | 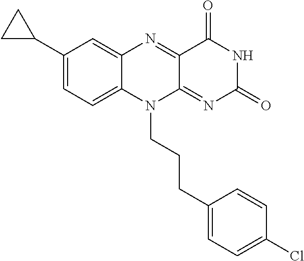 [SMILES: O=c1nc2n(CCCc3ccc(Cl)cc3)c3ccc(C4CC4)cc3nc-2c(=O)[nH]1] | 407.0 | 3.92 | D | Prepared using the synthesis of Example 1 starting with 4- cyclopropyl-2- nitroaniline and 1-(3- bromopropyl)-4- chlorobenzene | 10-(3-(4- chlorophenyl) propyl)-7- cyclopropyl- benzo[g] pteridine- 2,4(3H,10H)- dione |
| 18 | 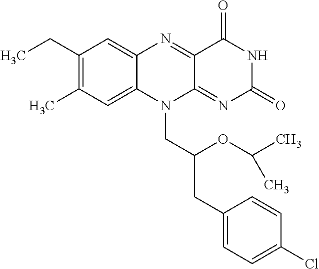 [SMILES: CCc1cc2nc3c(=O)[nH]c(=O)nc-3n(CC(Cc3ccc(Cl)cc3)OC(C)C)c2cc1C] | 467.4 | 4.84 | D | Prepared using synthesis of Example 17 starting from 2- bromo-5-ethyl-4- methylnitrobenzene and 3-(4- chlorophenyl)-2- isopropoxypropan-1- amine | 10-(3-(4- chlorophenyl)- 2- isopropoxy- propyl)-7-ethyl- 8- methylbenzo [g]pteridine- 2,4(3H,10H)- dione |
| 19 | 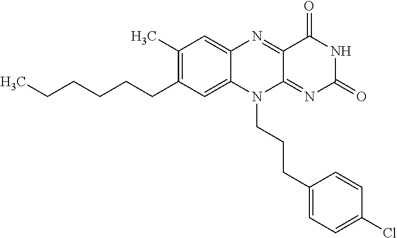 [SMILES: CCCCCCc1cc2c(cc1C)nc1c(=O)[nH]c(=O)nc-1n2CCCc1ccc(Cl)cc1] | 465.3 | 5.58 | G | Prepared using synthesis of Example 1 starting from 5-hexyl- 4-methyl-2-nitroanilin and 1-(3- bromopropyl)-4- chlorobenzene | 10-(3-(4- chlorophenyl) propyl)-8- hexyl-7- methylbenzo [g]pteridine- 2,4(3H,10H)- dione |
| 20 | 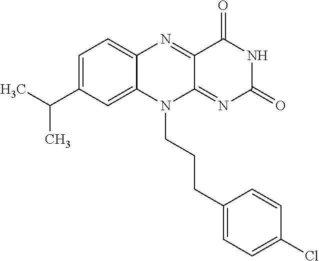 [SMILES: CC(C)c1ccc2nc3c(=O)[nH]c(=O)nc-3n(CCCc3ccc(Cl)cc3)c2c1] | 409.3 | 4.10 | D | Prepared using the synthesis of Example 1 starting with 5- isopropyl-2- nitroaniline and 1-(3- bromopropyl)-4- chlorobenzene | 10-(3-(4- chlorophenyl) propyl)-8- isopropylbenzo [g]pteridine- 2,4(3H,10H)- dione |
| 21 | 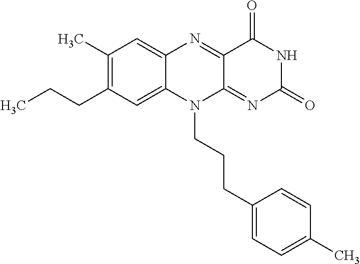 [SMILES: CCCc1cc2c(cc1C)nc1c(=O)[nH]c(=O)nc-1n2CCCc1ccc(C)cc1] | 403.4 | 4.37 | D | Prepared using the synthesis of Example 1 starting with 4- methyl-5-propyl-2- nitroaniline and 1-(3- bromopropyl)-4- methylbenzene | 7-methyl-8- propyl-10-(3- (p- tolyl)propyl) benzo[g] pteridine- 2,4(3H,10H)- dione |
| 22 | 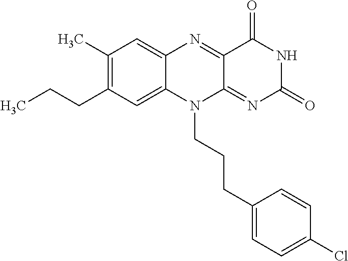 [SMILES: CCCc1cc2c(cc1C)nc1c(=O)[nH]c(=O)nc-1n2CCCc1ccc(Cl)cc1] | 423.2 | 4.25 | D | Prepared using the synthesis of Example 1 starting with 4- methyl-5-propyl-2- nitroaniline and 1-(3- bromopropyl)-4- chlorobenzene | 10-(3-(4- chlorophenyl) propyl)-7- methyl-8- propylbenzo [g]pteridine- 2,4(3H,10H)- dione |
| 23 | 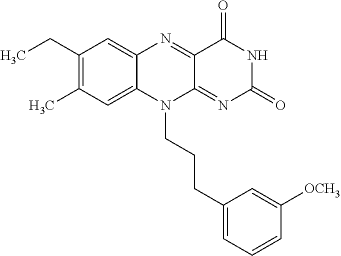 [SMILES: CCc1cc2nc3c(=O)[nH]c(=O)nc-3n(CCCc3cccc(OC)c3)c2cc1C] | 405.0 | 3.77 | D | Prepared using the synthesis of Example 1 starting with 4-ethyl- 5-methyl-2- nitroaniline and m-(3- bromopropyl)-Anisole | 7-ethyl-10- (3-(3- methoxyphen- yl)propyl)-8- methylbenzo [g]pteridine- 2,4(3H,10H)- dione |
| 24 | 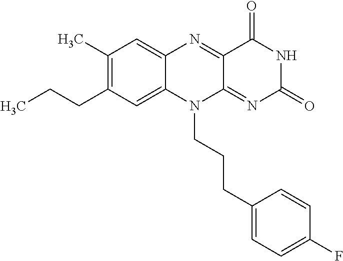 [SMILES: CCCc1cc2c(cc1C)nc1c(=O)[nH]c(=O)nc-1n2CCCc1ccc(F)cc1] | 407.3 | 3.99 | D | Prepared using the synthesis of Example 1 starting with 4- methyl-5-propyl-2- nitroaniline and 1-(3- bromopropyl)-4- fluorobenzene | 10-(3-(4- fluorophenyl) propyl)-7- methyl-8- propylbenzo [g]pteridine- 2,4(3H,10H)- dione |
| 25 | 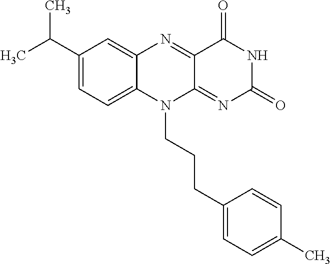 [SMILES: Cc1ccc(CCCn2c3nc(=O)[nH]c(=O)c-3nc3cc(C(C)C)ccc32)cc1] | 389.3 | 4.15 | D | Prepared using the synthesis of Example 1 starting with 4- isopropyl-2- nitroaniline and 1-(3- bromopropyl)-4- methylbenzene | 7-isopropyl- 10-(3-(p- tolyl)propyl) benzo[g] pteridine- 2,4(3H,10H)- dione |
| 26 | 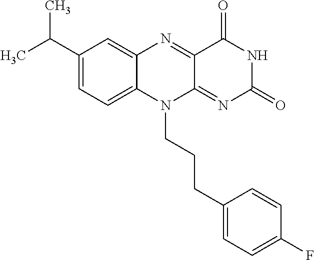 [SMILES: CC(C)c1ccc2c(c1)nc1c(=O)[nH]c(=O)nc-1n2CCCc1ccc(F)cc1] | 393.4 | 4.22 | G | Prepared using the synthesis of Example 1 starting with 4- isopropyl-2- nitroaniline and 1-(3- bromopropyl)-4- fluorobenzene | 10-(3-(4- fluorophenyl) propyl)-7- isopropylbenzo [g]pteridine- 2,4(3H,10H)- dione |
| 27 | 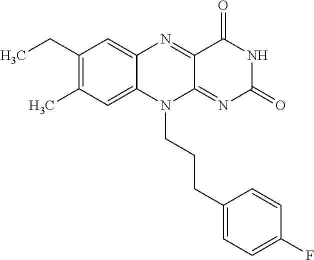 [SMILES: CCc1cc2nc3c(=O)[nH]c(=O)nc-3n(CCCc3ccc(F)cc3)c2cc1C] | 393.3 | 4.15 | G | Prepared using the synthesis of Example 1 starting with 5- methyl-2-nitro-4- vinylaniline and 1-(3- bromopropyl)-4- fluorobenzene | 7-ethyl-10- (3-(4- fluorophenyl) propyl) 8- methylbenzo [g]pteridine 2,4(3H,10H)- dione |
| 28 | 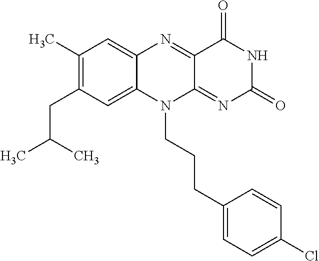 [SMILES: Cc1cc2nc3c(=O)[nH]c(=O)nc-3n(CCCc3ccc(Cl)cc3)c2cc1CC(C)C] | 437.0 | 4.57 | D | Prepared using the synthesis of Example 1 starting with 5- isobutyl-4-methyl-2- nitroaniline and 1-(3- bromopropyl)-4- chlorobenzene | 10-(3-(4- chlorophenyl) propyl)-8- isobutyl-7- methylbenzo [g]pteridine- 2,4(3H,10H)- dione |
| 29 | 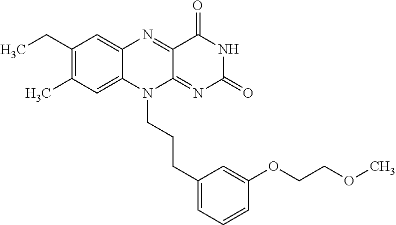 [SMILES: CCc1cc2nc3c(=O)[nH]c(=O)nc-3n(CCCc3cccc(OCCOC)c3)c2cc1C] | 449.3 | 3.69 | D | Prepared using the synthesis of example 1 starting with 4-ethyl- 5-methyl-2- nitroaniline and 1-(3- bromopropyl)-3-(2- methoxyethoxy)benzene | 7-ethyl-10- (3-(3-(2- methoxyeth- oxy)phenyl) propyl)-8- methylbenzo [g]pteridine- 2,4(3H,10H)- dione |
| 30 | 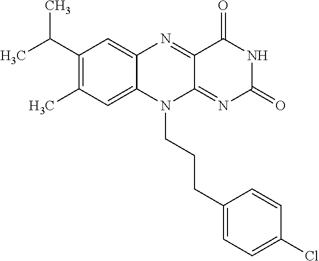 [SMILES: Cc1cc2c(cc1C(C)C)nc1c(=O)[nH]c(=O)nc-1n2CCCc1ccc(Cl)cc1] | 423.0 | 4.35 | D | Prepared using the synthesis of Example 1 starting with 4- isopropyl-5-methyl-2- nitroaniline and 1-(3- bromopropyl)-4- chlorobenzene | 10-(3-(4- chlorophenyl) propyl)-7- isopropyl-8- methylbenzo [g]pteridine- 2,4(3H,10H)- dione |
| 31 | 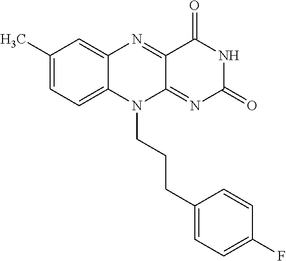 [SMILES: Cc1ccc2c(c1)nc1c(=O)[nH]c(=O)nc-1n2CCCc1ccc(F)cc1] | 365.4 | 3.38 | D | Prepared using the synthesis of Example 1 starting with 4- methyl-2-nitroaniline and 1-(3- bromopropyl)-4- fluorobenzene | 10-(3-(4- fluorophenyl) propyl)-7- methylbenzo [g]pteridine- 2,4(3H,10H)- dione |
| 32 | 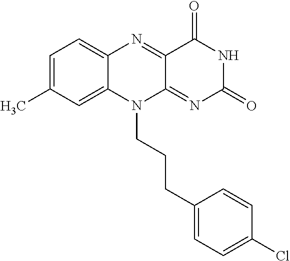 [SMILES: Cc1ccc2nc3c(=O)[nH]c(=O)nc-3n(CCCc3ccc(Cl)cc3)c2c1] | 381.3 | 3.60 | D | Prepared using the synthesis of Example 1 starting with 5- methyl-2-nitroaniline and 1-(3- bromopropyl)-4- chlorobenzene | 10-(3-(4- chlorophenyl) propyl)-8- methylbenzo [g]pteridine- 2,4(3H,10H)- dione |
| 33 | 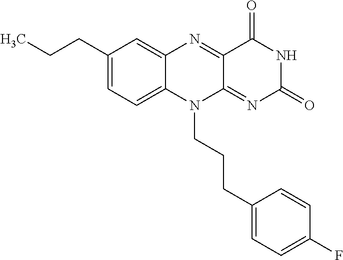 [SMILES: CCCc1ccc2c(c1)nc1c(=O)[nH]c(=O)nc-1n2CCCc1ccc(F)cc1] | 393.4 | 4.27 | G | Prepared using the synthesis of Example 1 starting with2-nitro-4- propylaniline and 1-(3- bromopropyl)-4- fluorobenzene | 10-(3-(4- fluorophenyl) propyl)-7- propylbenzo [g]pteridine- 2,4(3H,10H)- dione |
| 34 | 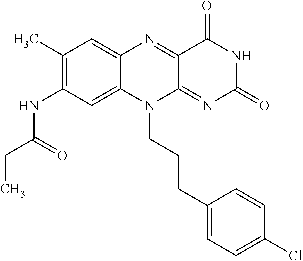 [SMILES: CCC(=O)Nc1cc2c(cc1C)nc1c(=O)[nH]c(=O)nc-1n2CCCc1ccc(Cl)cc1] | 452.2 | 3.53 | D | Prepared using the synthesis of example 23 starting with 8- amino-10-[3-(4- chlorophenyl)propyl]- 7- methylbenzo[g]pteridine- 2,4(3H,10H)-dione and propanoyl chloride | N-(10-(3-(4- chlorophenyl) propyl)-7- methyl-2,4- dioxo- 2,3,4,10- tetrahydro- benzo[g] pteridin-8- yl)propion- amide |
| 35 | 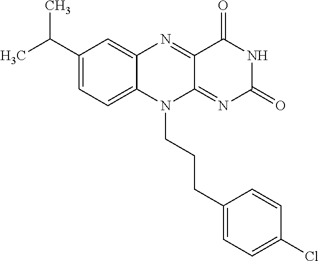 [SMILES: CC(C)c1ccc2c(c1)nc1c(=O)[nH]c(=O)nc-1n2CCCc1ccc(Cl)cc1] | 409.3 | 4.47 | G | Prepared using the synthesis of Example 1 starting with 4- isopropyl-2- nitroaniline and 1-(3- bromopropyl)-4- chlorobenzene | 10-(3-(4- chlorophenyl) propyl)-7- isopropylbenzo [g]pteridine- 2,4(3H,10H)- dione |
| 36 | 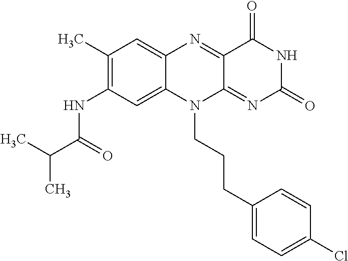 [SMILES: Cc1cc2nc3c(=O)[nH]c(=O)nc-3n(CCCc3ccc(Cl)cc3)c2cc1NC(=O)C(C)C] | 464.5 (ES−) | 3.77 | D | Prepared using the synthesis of example 23 starting with 8- amino-10-[3-(4- chlorophenyl)propyl]- 7- methylbenzo[g]pteridine- 2,4(3H,10H)-dione and isobutryl chloride | N-(10-(3-(4- chlorophenyl) propyl)-7- methyl-2,4- dioxo- 2,3,4,10- tetrahydrobenzo [g]pteridin- 8- yl)isobutyr- amide |
| 37 | 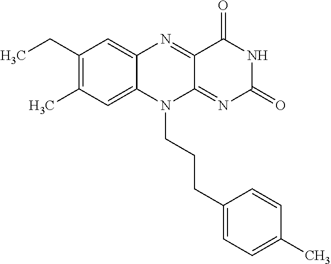 [SMILES: CCc1cc2nc3c(=O)[nH]c(=O)nc-3n(CCCc3ccc(C)cc3)c2cc1C] | 389.0 | 4.09 | D | Prepared using the synthesis of Example 1 starting with 4-ethyl- 5-methyl-2- nitroaniline and 1-(3- bromopropyl)-4- methylbenzene | 7-ethyl-8- methyl-10- (3-(p- tolyl)propyl) benzo[g] pteridine- 2,4(3H,10H)- dione |
| 38 | 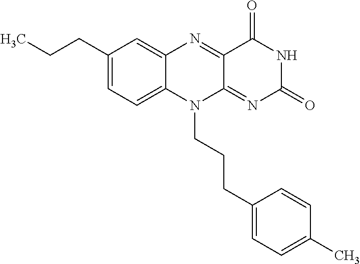 [SMILES: CCCc1ccc2c(c1)nc1c(=O)[nH]c(=O)nc-1n2CCCc1ccc(C)cc1] | 389.3 | 4.46 | G | Prepared using the synthesis of Example 1 starting with 4-propyl- 2-nitroaniline and 1- (3-bromopropyl)-4- methylbenzene | 7-propyl-10- (3-(p- tolyl)propyl) benzo[g] pteridine- 2,4(3H,10H)- dione |
| 39 | 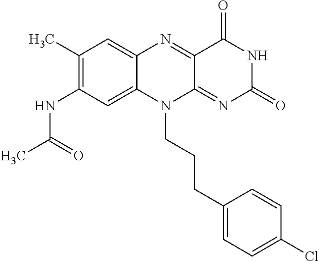 [SMILES: CC(=O)Nc1cc2c(cc1C)nc1c(=O)[nH]c(=O)nc-1n2CCCc1ccc(Cl)cc1] | 438.2 | 3.23 | D | Prepared using the synthesis of example 23 starting with 8- amino-10-[3-(4- chlorophenyl)propyl]- 7- methylbenzo[g]pteridine- 2,4(3H,10H)-dione and acetyl chloride | N-(10-(3-(4- chlorophenyl) propyl)-7- methyl-2,4- dioxo- 2,3,4,10- tetrahydro- benzo [g]pteridin- 8- yl)acetamide |
| 40 | 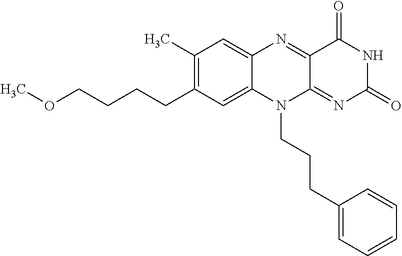 [SMILES: COCCCCc1cc2c(cc1C)nc1c(=O)[nH]c(=O)nc-1n2CCCc1ccccc1] | 433.4 | 4.10 | G | Prepared using the synthesis of Example 1 starting with 5-(4- methoxybutyl)-4- methyl-N-(3- phenylpropyl)benzene- 1,2-diamine, intermediate N. | 8-(4- methoxybutyl)- 7-methyl- 10-(3- phenylpropyl) benzo[g] pteridine- 2,4(3H,10H)- dione |
| 41 | 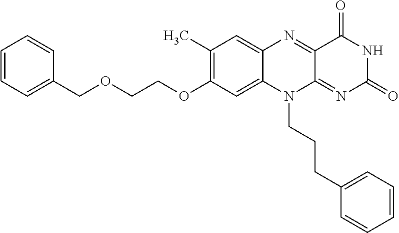 [SMILES: Cc1cc2nc3c(=O)[nH]c(=O)nc-3n(CCCc3ccccc3)c2cc1OCCOCc1ccccc1] | 497.4 | 4.59 | G | Prepared using the synthesis of Example 11 starting with 8- chloro-7-methyl-10- (3- phenylpropyl)benzo[g] pteridine-2,4(3H,10H)- dione and 2- (phenylmethoxy)- ethanol | 8-(2- (benzyloxy) ethoxy)-7- methyl-10- (3- phenylpropyl) benzo[g] pteridine- 2,4(3H,10H)- dione |
| 42 | 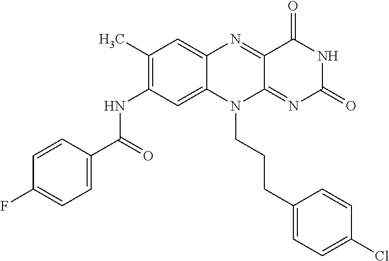 [SMILES: Cc1cc2nc3c(=O)[nH]c(=O)nc-3n(CCCc3ccc(Cl)cc3)c2cc1NC(=O)c1ccc(F)cc1] | 518.3 | 4.05 | D | Prepared using the synthesis of example 23 starting with 8- amino-10-[3-(4- chlorophenyl)propyl]- 7- methylbenzo[g]pteridine- 2,4(3H,10H)-dione and 4-fluorobenzoyl chloride | N-(10-(3-(4- chlorophenyl) propyl)-7- methyl-2,4- dioxo- 2,3,4,10- tetrahydro- benzo [g]pteridin- 8-yl)-4- fluorobenz- amide |
| 43 | 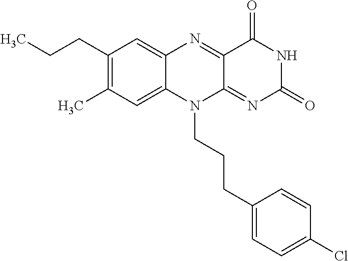 [SMILES: CCCc1cc2nc3c(=O)[nH]c(=O)nc-3n(CCCc3ccc(Cl)cc3)c2cc1C] | 423.3 | 4.40 | D | Prepared using the synthesis of Example 1 starting with (4-allyl-5- methyl-2-nitro- phenyl)-(3-(4- chlorophenyl)propyl)- amine prepared as described in the preparation of intermediate N. | 10-(3-(4- chlorophenyl) propyl)-8- methyl-7- propylbenzo [g]pteridine- 2,4(3H,10H)- dione |
| 44 | 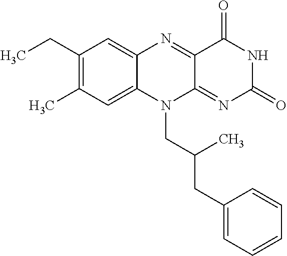 [SMILES: CCc1cc2nc3c(=O)[nH]c(=O)nc-3n(CC(C)Cc3ccccc3)c2cc1C] | 389.0 | 3.99 | D | Prepared using the synthesis of Example 1 starting with 4-ethyl- 5-methyl-2- nitroaniline and (3- bromo-2- methylpropyl)benzene | 7-ethyl-8- methyl-10- (2-methyl-3- phenylpropyl) benzo[g] pteridine- 2,4(3H,10H)- dione |
| 45 | 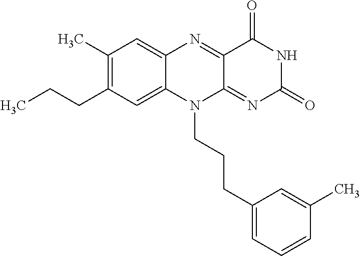 [SMILES: CCCc1cc2c(cc1C)nc1c(=O)[nH]c(=O)nc-1n2CCCc1cccc(C)c1] | 403.3 | 4.62 | G | Prepared using the synthesis of Example 1 starting with (5-allyl-4- methyl-2-nitro- phenyl)-(3-m-tolyl- propyl)-amine prepared as described in the preparation of intermediate N. | 7-methyl-8- propyl-10-(3- (m- tolyl)propyl) benzo[g] pteridine- 2,4(3H,10H)- dione |
| 46 | 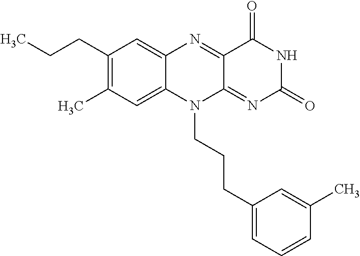 [SMILES: CCCc1cc2nc3c(=O)[nH]c(=O)nc-3n(CCCc3cccc(C)c3)c2cc1C] | 403.4 | 4.34 | D | Prepared using the synthesis of Example 1 starting with (4-allyl-5- methyl-2-nitro- phenyl)-(3-m-tolyl- propyl)-amine prepared as described in the preparation of intermediate N. | 8-methyl-7- propyl-10-(3- (m- tolyl)propyl) benzo[g] pteridine 2,4(3H,10H)- dione |
| 47 | 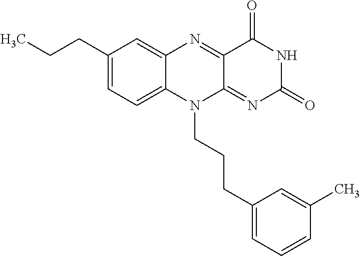 [SMILES: CCCc1ccc2c(c1)nc1c(=O)[nH]c(=O)nc-1n2CCCc1cccc(C)c1] | 389.3 | 4.43 | G | Prepared using the synthesis of Example 1 starting with 2-nitro- 4-propylaniline and 1- (3-bromopropyl)-3- methylbenzene | 7-propyl-10- (3-(m- tolyl)propyl) benzo[g] pteridine- 2,4(3H,10H)- dione |
| 48 | 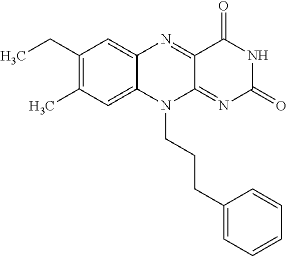 [SMILES: CCc1cc2nc3c(=O)[nH]c(=O)nc-3n(CCCc3ccccc3)c2cc1C] | 375.0 | 3.85 | D | Prepared using the synthesis of Example 1 starting with 4-ethyl- 5-methyl-2- nitroaniline and 1- Bromo-3- phenylpropane | 7-ethyl-8- methyl-10- (3- phenylpropyl) benzo[g] pteridine- 2,4(3H,10H)- dione |
| 49 | 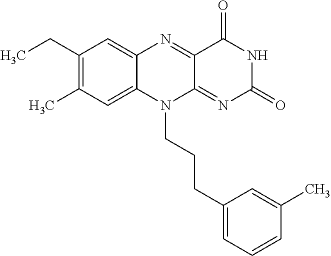 [SMILES: CCc1cc2nc3c(=O)[nH]c(=O)nc-3n(CCCc3cccc(C)c3)c2cc1C] | 389.0 | 4.05 | D | Prepared using the synthesis of Example 1 starting with 4-ethyl- 5-methyl-2- nitroaniline and 1-(3- bromopropyl)-3- methylbenzene | 7-ethyl-8- ethyl-10- (3-(m- tolyl)propyl) benzo[g] pteridine- 2,4(3H,10H)- dione |
| 50 | 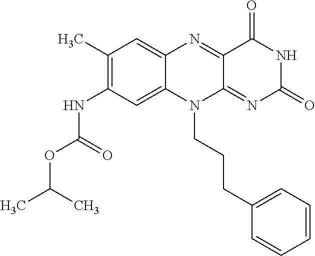 [SMILES: Cc1cc2nc3c(=O)[nH]c(=O)nc-3n(CCCc3ccccc3)c2cc1NC(=O)OC(C)C] | 448.2 | 3.68 | D | Prepared using the synthesis of Example 23 and using isopropyl chloroformate | isopropyl (7- methyl-2,4- dioxo-10-(3- phenylpropyl)- 2,3,4,10- tetrahydro- benzo [g]pteridin- 8- yl)carbamate |
| 51 | 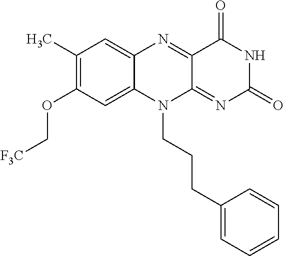 [SMILES: Cc1cc2nc3c(=O)[nH]c(=O)nc-3n(CCCc3ccccc3)c2cc1OCC(F)(F)F] | 445.2 | 4.29 | G | Prepared using the synthesis of Example 11 starting with 8- chloro-7-methyl-10- (3- phenylpropyl)benzo[g] pteridine-2,4(3H,10H)- dione and 2,2,2- trifluoroethanol | 7-methyl-10- (3- phenylpropyl)- 8-(2,2,2- trifluoroeth- oxy)benzo[g] pteridine- 2,4(3H,10H)- dione |
| 52 | 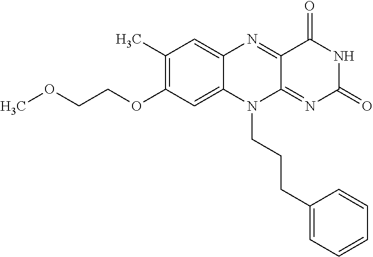 [SMILES: COCCOc1cc2c(cc1C)nc1c(=O)[nH]c(=O)nc-1n2CCCc1ccccc1] | 421.3 | 3.67 | G | Prepared using the synthesis of Example 11 starting with 8- chloro-7-methyl-10- (3- phenylpropyl)benzo[g] pteridine-2,4(3H,10H)- dione and 2- Methoxyethanol | 8-(2- methoxyeth- oxy)-7-methyl- 10-(3- phenylpropyl) benzo[g] pteridine- 2,4(3H,10H)- dione |
| 53 | 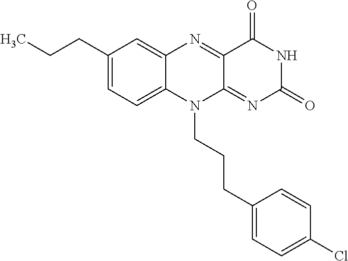 [SMILES: CCCc1ccc2c(c1)nc1c(=O)[nH]c(=O)nc-1n2CCCc1ccc(Cl)cc1] | 409.2 | 4.51 | G | Prepared using the synthesis of Example 1 starting with 2-nitro- 4-propylaniline and 1- (3-bromopropyl)-4- chlorobenzene | 10-(3-(4- chlorophenyl) propyl)-7- propylbenzo [g]pteridine- 2,4(3H,10H)- dione |
| 54 | 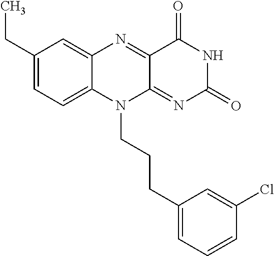 [SMILES: CCc1ccc2c(c1)nc1c(=O)[nH]c(=O)nc-1n2CCCc1cccc(Cl)c1] | 395.2 | 3.81 | D | Prepared using the synthesis of Example 1 starting with 4-ethyl- 2-nitroaniline and 1- (3-bromopropyl)-3- chlorobenzene | 10-(3-(3- chlorophenyl) propyl)-7- ethylbenzo [g]pteridine- 2,4(3H,10H)- dione |
| 55 | 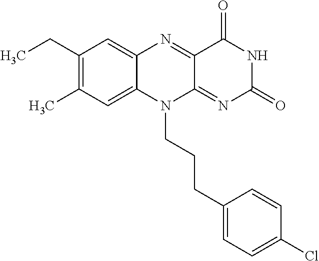 [SMILES: CCc1cc2nc3c(=O)[nH]c(=O)nc-3n(CCCc3ccc(Cl)cc3)c2cc1C] | 409.2 | 4.02 | D | Prepared using the synthesis of Example 1 starting with 4-ethyl- 5-methyl-2- nitroaniline and 1-(3- bromopropyl)-4- chlorobenzene | 10-(3-(4- chlorophenyl) propyl)-7- ethyl-8- methylbenzo [g]pteridine- 2,4(3H,10H)- dione |
| 56 | 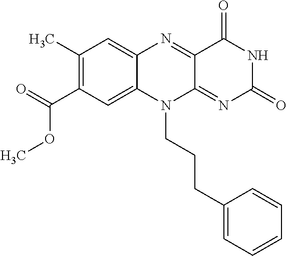 [SMILES: COC(=O)c1cc2c(cc1C)nc1c(=O)[nH]c(=O)nc-1n2CCCc1ccccc1] | 405.3 | 3.46 | D | Prepared using the synthesis of Example 1 starting with methyl 5-amino-2-methyl-4- nitrobenzoate and 1- bromo-3- phenylpropane | methyl 7- methyl-2,4- dioxo-10-(3- phenylpropyl)- 2,3,4,10- tetrahydro- benzo [g]pteridine- 8- carboxylate |
| 57 | 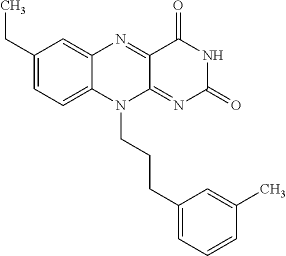 [SMILES: CCc1ccc2c(c1)nc1c(=O)[nH]c(=O)nc-1n2CCCc1cccc(C)c1] | 375.4 | 4.11 | G | Prepared using the synthesis of Example 1 starting with 4-ethyl- 2-nitroaniline and 1- (3-bromopropyl)-3- methylbenzene | 7-ethyl-10- (3-(m- tolyl)propyl) benzo[g] pteridine- 2,4(3H,10H)- dione |
| 58 | 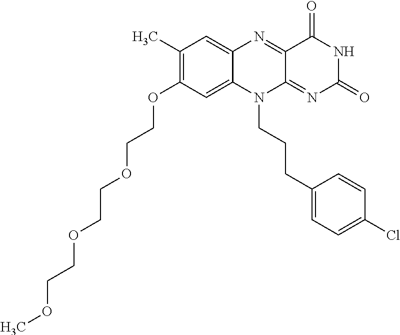 [SMILES: COCCOCCOCCOc1cc2c(cc1C)nc1c(=O)[nH]c(=O)nc-1n2CCCc1ccc(Cl)cc1] | 545.0 | 3.63 | D | Prepared using the synthesis of Example 11 starting with 8- chloro-7-methyl-10- (3-(4- chlorophenyl)propyl) benzo[g]pteridine- 2,4(3H,10H)-dione and 2-[2-(2- methoxyethoxy)ethoxy]- ethanol | 10-(3-(4- chlorophenyl) propyl)-8-(2- (2-(2- methoxyeth- oxy)ethoxy) ethoxy)-7- methylbenzo [g]pteridine- 2,4(3H,10H)- dione |
| 59 | 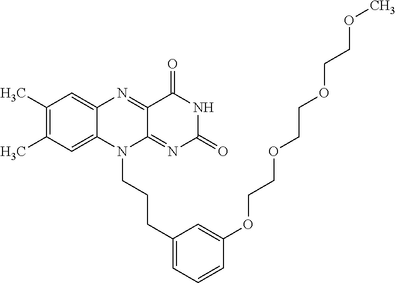 | 523.0 | 3.38 | D | Prepared using the synthesis of Example 1 starting with 4,5- dimethyl-2- nitroaniline and 1-(3- bromopropyl)-3-{2-[2- (2- methoxyethoxy)ethoxy] ethoxy}benzene | 10-(3-(3-(2- (2-(2- methoxyeth- oxy)ethoxy) ethoxy)phenyl) propyl)-7,8- dimethylbenzo [g]pteridine- 2,4(3H,10H)- dione |
| 60 | 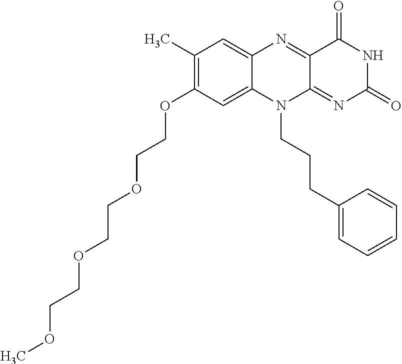 [SMILES: COCCOCCOCCOc1cc2c(cc1C)nc1c(=O)[nH]c(=O)nc-1n2CCCc1ccccc1] | 509.2 | 3.61 | G | Prepared using the synthesis of Example 11 starting with 8- chloro-7-methyl-10- (3- phenylpropyl)benzo[g] pteridine-2,4(3H,10H)- dione and 2-[2-(2- methoxyethoxy)ethoxy]- ethanol | 8-(2-(2-(2- methoxyeth- oxy)ethoxy) ethoxy)-7- methyl-10- (3- phenylpropyl) benzo[g] pteridine- 2,4(3H,10H)- dione |
| 61 | 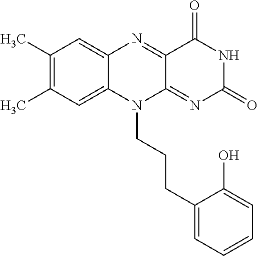 [SMILES: Cc1cc2nc3c(=O)[nH]c(=O)nc-3n(CCCc3ccccc3O)c2cc1C] | 377.2 | 3.16 | D | Prepared using the synthesis of Example 8, starting with 10-(3- (2- methoxyphenyl)propyl)- 7,8- dimethylbenzo[g] pteridine- 2,4(3H,10H)- dione | 10-(3-(2- hydroxyphen- yl)propyl)- 7,8- dimethylbenzo [g]pteridine- 2,4(3H,10H)- dione |
| 62 | 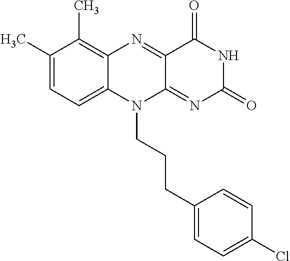 [SMILES: Cc1ccc2c(nc3c(=O)[nH]c(=O)nc-3n2CCCc2ccc(Cl)cc2)c1C] | 395.1 | 3.98 | D | Prepared using the synthesis of Example 4, starting with 3,4- Dimethyl-2- nitroaniline and intermediate B | 10-(3-(4- chlorophenyl) propyl)-6,7- dimethylbenzo [g]pteridine- 2,4(3H,10H)- dione |
| 63 | 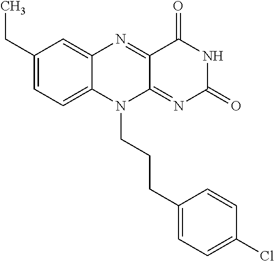 [SMILES: CCc1ccc2c(c1)nc1c(=O)[nH]c(=O)nc-1n2CCCc1ccc(Cl)cc1] | 395.0 | 3.95 | D | Prepared using the synthesis of Example 1, starting with 4- Ethyl-2-nitroaniline and intermediate B | 10-(3-(4- chlorophenyl) propyl)-7- ethylbenzo[g] pteridine- 2,4(3H,10H)- dione |
| 64 | 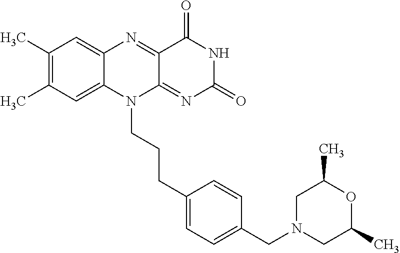 | 488.2 | 2.91 | G | Prepared using the synthesis of Example 1 starting with N-(3-{4- [(2,6- Dimethylmorpholin-4- yl)methyl]phenyl}prop- yl)-4,5-dimethyl-2- nitroaniline, intermediate P | 10-(3-(4- (((2S,6R)-2,6- dimethyl- morpholino) methyl) phenyl)prop- yl)-7,8- dimethylbenzo [g]pteridine- 2,4(3H,10H)- dione |
| 65 | 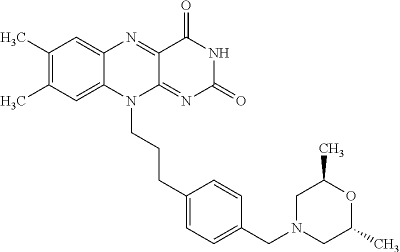 | 488.3 | 2.86 | G | Prepared using the synthesis of Example 1 starting with N-(3-{4- [(2,6- Dimethylmorpholin-4- yl)methyl]phenyl}prop- yl)-4,5-dimethyl-2- nitroaniline, intermediate P | 10-(3-(4- (((2R,6R)-2,6- dimethyl- morpholino) methyl) phenyl)prop- yl)-7,8- dimethylbenzo [g]pteridine- 2,4(3H,10H)- dione |
| 66 | 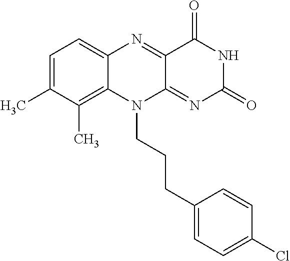 [SMILES: Cc1ccc2nc3c(=O)[nH]c(=O)nc-3n(CCCc3ccc(Cl)cc3)c2c1C] | 395.1 | 3.76 | D | Prepared using the synthesis of Example 1, starting with 5,6- Dimethyl-2- nitroaniline and intermediate B | 10-(3-(4- chlorophenyl) propyl)-8,9- dimethylbenzo [g]pteridine- 2,4(3H,10H)- dione |
| 67 | 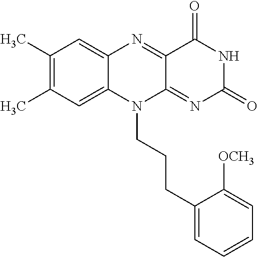 [SMILES: COc1ccccc1CCCn1c2nc(=O)[nH]c(=O)c-2nc2cc(C)c(C)cc21] | 391.1 | 3.60 | D | Prepared using the synthesis of Example 1, starting with 4,5- Dimethyl-2- nitroaniline and 3-(2- methoxyphenyl)propyl bromide | 10-(3-(2- methoxyphen- yl)propyl)- 7,8- dimethylbenzo [g]pteridine 2,4(3H,10H)- dione |
| 68 | 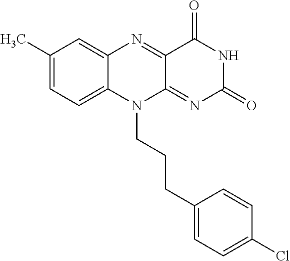 [SMILES: Cc1ccc2c(c1)nc1c(=O)[nH]c(=O)nc-1n2CCCc1ccc(Cl)cc1] | 381.1 | 3.68 | D | Prepared using the synthesis of Example 1, starting with 4- methyl-2-nitroaniline and intermediate B | 10-(3-(4- chlorophenyl) propyl)-7- methylbenzo [g]pteridine- 2,4(3H,10H)- dione |
| 69 | 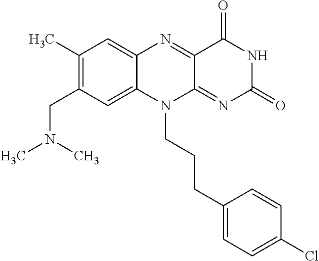 [SMILES: Cc1cc2nc3c(=O)[nH]c(=O)nc-3n(CCCc3ccc(Cl)cc3)c2cc1CN(C)C] | 438.0 | 2.65 | D | Prepared using the synthesis of Example 19 starting with 8- (bromomethyl)-10-[3- (4- chlorophenyl)propyl]- 7- methylbenzo[g]pteridine- 2,4(3H,10H)-dione and dimethylamine | 10-(3-(4- chlorophenyl) propyl)-8- ((dimethyl- amino)methyl)- 7- methylbenzo [g]pteridine- 2,4(3H,10H)- dione |
| 70 | 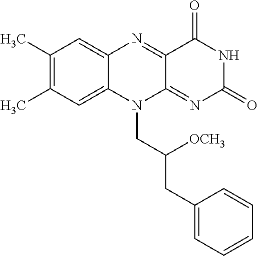 [SMILES: COC(Cc1ccccc1)Cn1c2nc(=O)[nH]c(=O)c-2nc2cc(C)c(C)cc21] | 391.3 | 3.52 | D | Prepared using synthesis of Example 17 starting from 2- bromo-4,5- dimethylnitrobenzene and 2-methoxy-3- phenylpropan-1- amine | 10-(2- methoxy-3- phenylpropyl)- 7,8- dimethylbenzo [g]pteridine- 2,4(3H,10H)- dione |
| 71 | 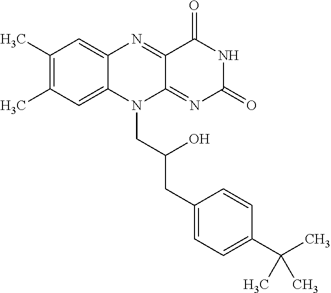 [SMILES: Cc1cc2nc3c(=O)[nH]c(=O)nc-3n(CC(O)Cc3ccc(C(C)(C)C)cc3)c2cc1C] | 433.2 | 3.98 | D | Prepared using synthesis of Example 22 starting from 4,5-dimethyl-2- nitroaniline and 2-(4- tertbutylbenzyl)oxirane | 10-(3-(4- (tert- butyl)phenyl)- 2- hydroxyprop- yl)-7,8- dimethylbenzo [g]pteridine- 2,4(3H,10H)- dione |
| 72 | 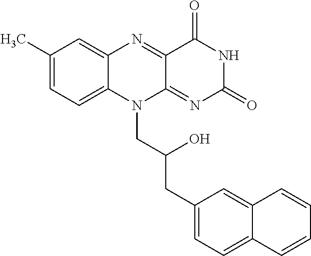 [SMILES: Cc1ccc2c(c1)nc1c(=O)[nH]c(=O)nc-1n2CC(O)Cc1ccc2ccccc2c1] | 413.3 | 3.37 | D | Prepared using synthesis of Example 22 starting from 4- methyl-2-nitroaniline and 2-(2- naphthylmethyl)oxirane | 10-(2- hydroxy-3- (naphthalen- 2-yl)propyl)- 7- methylbenzo [g]pteridine- 2,4(3H,10H)- dione |
| 73 | 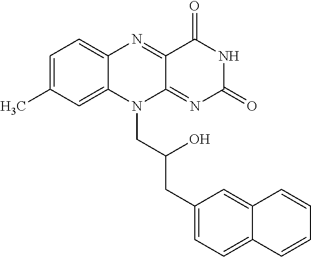 [SMILES: Cc1ccc2nc3c(=O)[nH]c(=O)nc-3n(CC(O)Cc3ccc4ccccc4c3)c2c1] | 413.2 | 3.38 | D | Prepared using synthesis of Example 22 starting from 5-methyl-2- nitroaniline and 2-(2- naphthylmethyl)oxirane | 10-(2- hydroxy-3- (naphthalen- 2-yl)propyl)- 8- methylbenzo [g]pteridine- 2,4(3H,10H)- dione |
| 74 | 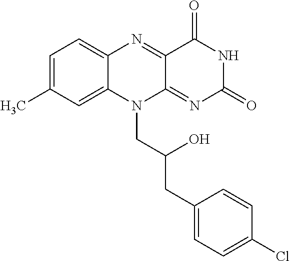 [SMILES: Cc1ccc2nc3c(=O)[nH]c(=O)nc-3n(CC(O)Cc3ccc(Cl)cc3)c2c1] | 397.1 | 3.26 | D | Prepared using synthesis of Example 22 starting from 5- Methyl-2-nitroaniline and 2-(4- chlorobenzyl)oxirane | 10-(3-(4- chlorophenyl)- 2- hydroxyprop- yl)-8- methylbenzo [g]pteridine- 2,4(3H,10H)- dione |
| 75 | 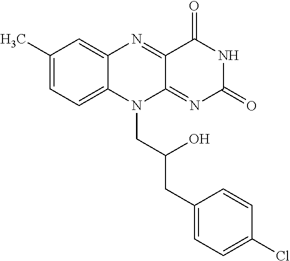 [SMILES: Cc1ccc2c(c1)nc1c(=O)[nH]c(=O)nc-1n2CC(O)Cc1ccc(Cl)cc1] | 397.0 | 3.27 | D | Prepared using synthesis of Example 22 starting from 4- Methyl-2-nitroaniline and 2-(4- chlorobenzyl)oxirane prepared as in intermediate H | 10-(3-(4- chlorophenyl)- 2- hydroxyprop- yl)-7- methylbenzo [g]pteridine- 2,4(3H,10H)- dione |
| 76 | 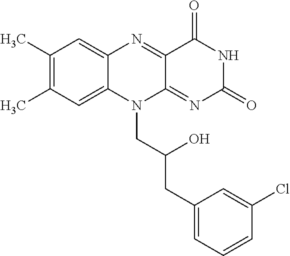 [SMILES: Cc1cc2nc3c(=O)[nH]c(=O)nc-3n(CC(O)Cc3cccc(Cl)c3)c2cc1C] | 411.0 | 3.40 | D | Prepared using synthesis of Example 22 starting from 4,5- Dimethyl-2- nitroaniline and 2-(3- chlorobenzyl)oxirane | 10-(3-(3- chlorophenyl)- 2- hydroxyprop- yl)-7,8- dimethylbenzo [g]pteridine- 2,4(3H,10H)- dione |
| 77 | 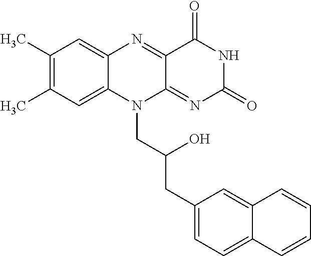 [SMILES: Cc1cc2nc3c(=O)[nH]c(=O)nc-3n(CC(O)Cc3ccc4ccccc4c3)c2cc1C] | 427.3 | 3.53 | D | Prepared using synthesis of Example 22 starting from 4,5- dimethyl-2- nitroaniline and 2-(2- naphthylmethyl)oxirane | 10-(2- hydroxy-3- (naphthalen- 2-yl)propyl)- 7,8- dimethylbenzo [g]pteridine- 2,4(3H,10H)- dione |
| 78 | 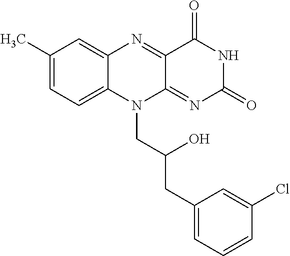 [SMILES: Cc1ccc2c(c1)nc1c(=O)[nH]c(=O)nc-1n2CC(O)Cc1cccc(Cl)c1] | 397.0 | 3.21 | D | Prepared using synthesis of Example 22 starting from 4- Methyl-2-nitroaniline and 2-(3- chlorobenzyl)oxirane prepared as in intermediate H | 10-(3-(3- chlorophenyl)- 2- hydroxyprop- yl)-7- methylbenzo [g]pteridine- 2,4(3H,10H)- dione |
| 79 | 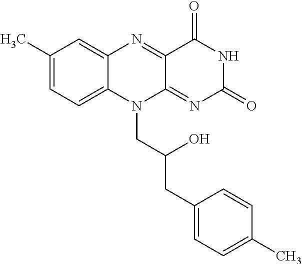 | 377.0 | 3.16 | D | Prepared using synthesis of Example 22 starting from 4- Methyl-2-nitroaniline and 2-(4- methylbenzyl)oxirane prepared as in intermediate H | 10-(2- hydroxy-3-(p- tolyl)propyl)- 7- methylbenzo [g]pteridine- 2,4(3H,10H)- dione |
| 80 | 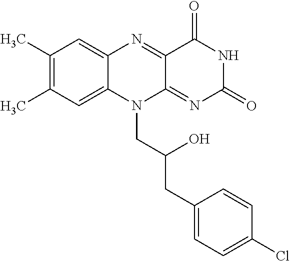 [SMILES: Cc1cc2nc3c(=O)[nH]c(=O)nc-3n(CC(O)Cc3ccc(Cl)cc3)c2cc1C] | 411.0 | 3.39 | D | Prepared using synthesis of Example 22 starting from 4,5- dimethyl-2- nitroaniline and 2-(4- chlorobenzyl)oxirane | 10-(3-(4- chlorophenyl)- 2- hydroxyprop- yl)-7,8- dimethylbenzo [g]pteridine- 2,4(3H,10H)- dione |
| 81 | 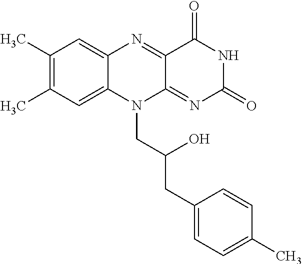 | 391.1 | 3.30 | D | Prepared using synthesis of Example 22 starting from 4,5- dimethyl-2- nitroaniline and 2-(4- methylbenzyl)oxirane | 10-(2- hydroxy-3-(p- tolyl)propyl)- 7,8- dimethylbenzo [g]pteridine- 2,4(3H,10H)- dione |
| 82 | 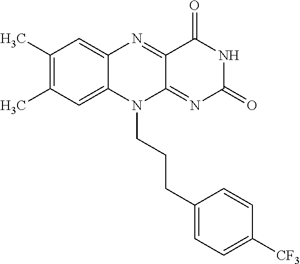 [SMILES: Cc1cc2nc3c(=O)[nH]c(=O)nc-3n(CCCc3ccc(C(F)(F)F)cc3)c2cc1C] | 429.0 | 3.91 | D | Prepared using synthesis of Example 1 starting from 4,5- dimethyl-2- nitroaniline and 1-(3- bromopropyl)-4- (trifluoromethyl)benz- ene | 7,8-dimethyl- 10-(3-(4- (trifluorometh- yl)phenyl) propyl)benzo[g] pteridine- 2,4(3H,10H)- dione |
| 83 | 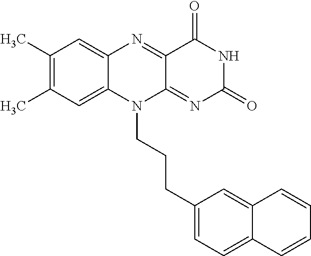 [SMILES: Cc1cc2nc3c(=O)[nH]c(=O)nc-3n(CCCc3ccc4ccccc4c3)c2cc1C] | 411.1 | 3.93 | D | Prepared using synthesis of Example 1 starting from 4,5- dimethyl-2- nitroaniline and 2-(3- bromopropyl)naphtha- lene | 7,8-dimethyl- 10-(3- (naphthalen- 2- yl)propyl)benzo [g]pteridine- 2,4(3H,10H)- dione |
| 84 | 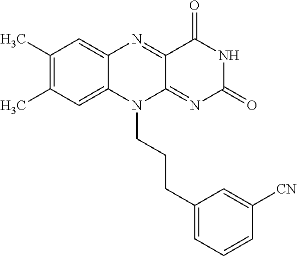 [SMILES: Cc1cc2nc3c(=O)[nH]c(=O)nc-3n(CCCc3cccc(C#N)c3)c2cc1C] | 386.0 | 3.28 | D | Prepared using the synthesis of Example 4 starting with 4,5- Dimethyl-o- phenylenediamine and 3-(3- bromopropyl)benzo- nitrile | 3-(3-(7,8- dimethyl-2,4- dioxo-3,4- dihydrobenzo [g]pteridin- 10(2H)- yl)propyl)benzo- nitrile |
| 85 | 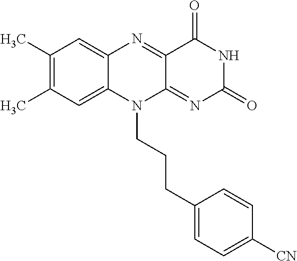 [SMILES: Cc1cc2nc3c(=O)[nH]c(=O)nc-3n(CCCc3ccc(C#N)cc3)c2cc1C] | 386.0 | 3.28 | D | Prepared using synthesis of Example 4 starting from 4,5- Dimethyl-o- phenylenediamine and 4-(3- bromopropyl)benzo- nitrile | 4-(3-(7,8- dimethyl-2,4- dioxo-3,4- dihydrobenzo [g]pteridin- 10(2H)- yl)propyl)benzo- nitrile |
| 86 | 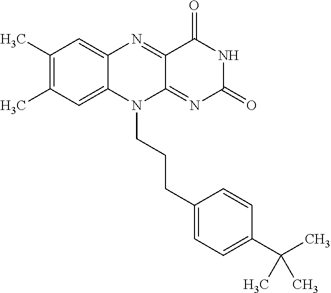 [SMILES: Cc1cc2nc3c(=O)[nH]c(=O)nc-3n(CCCc3ccc(C(C)(C)C)cc3)c2cc1C] | 417.2 | 4.46 | D | Prepared using synthesis of Example 4 starting from 4,5-Dimethyl-o- phenylenediamine and 1-(3-bromopropyl)-4- tert-butylbenzene which was prepared as in intermediate E, starting with 4- tertbutylbenzaldehyde | 10-(3-(4- (tert- butyl)phenyl) propyl)-7,8- dimethylbenzo [g]pteridine- 2,4(3H,10H)- dione |
| 87 | 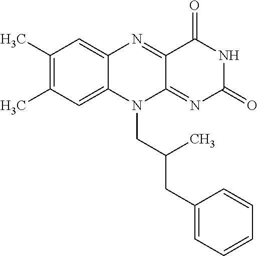 [SMILES: Cc1cc2nc3c(=O)[nH]c(=O)nc-3n(CC(C)Cc3ccccc3)c2cc1C] | 375.1 | 3.98 | G | Prepared using synthesis of Example 4 starting from 4,5- Dimethyl-o- phenylenediamine and (3-bromo-2- methylpropyl)benzene | 7,8-dimethyl- 10-(2- methyl-3- phenylpropyl) benzo[g] pteridine- 2,4(3H,10H)- dione |
| 88 | 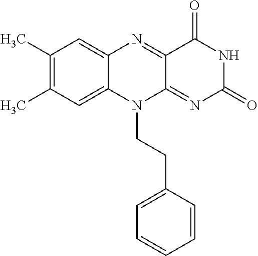 [SMILES: Cc1cc2nc3c(=O)[nH]c(=O)nc-3n(CCc3ccccc3)c2cc1C] | 347.2 | 4.19 | D | Prepared using synthesis of example 1 starting with 4,5- dimethyl-2-nitro-N-(2- phenylethyl)aniline | 7,8-dimethyl- 10- phenethylbenzo [g]pteridine- 2,4(3H,10H)- dione |
| 89 | 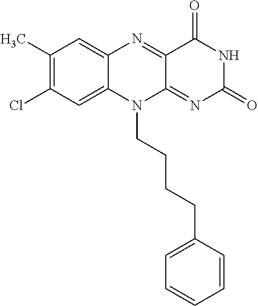 [SMILES: Cc1cc2nc3c(=O)[nH]c(=O)nc-3n(CCCCc3ccccc3)c2cc1Cl] | 395.1 | 5.91 | F | Prepared using synthesis of Example 9 starting from 4,5- dimethyl-2- nitroaniline and (4- bromobutyl)benzene | 8-chloro-7- methyl 10- (4- phenylbutyl) benzo[g] pteridine- 2,4(3H,10H)- dione |
| 90 | 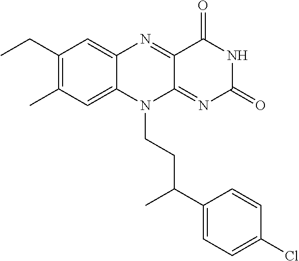 [SMILES: CCc1cc2nc3c(=O)[nH]c(=O)nc-3n(CCC(C)c3ccc(Cl)cc3)c2cc1C] | 423.1 | 4.23 | D | Prepared by the synthesis of example 1 starting from 4-ethyl- 5-methyl-2- nitroaniline and 1-(3- bromo-1- methylpropyl)-4- chlorobenzene | 10-[3-(4- chlorophenyl) butyl]-7- ethyl-8- methylbenzo [g]pteridine- 2,4(3H,10H)- dione |
| 91 | 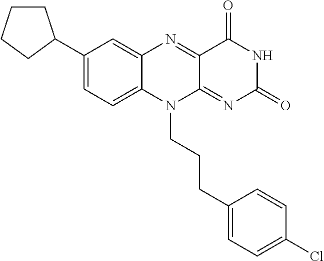 [SMILES: O=c1nc2n(CCCc3ccc(Cl)cc3)c3ccc(C4CCCC4)cc3nc-2c(=O)[nH]1] | 435.1 | 4.48 | D | Prepared by the synthesis of example 1 starting from 4- cyclopentyl-2- nitroaniline and 1-(3- bromopropyl)-4- chlorobenzene | 10-[3-(4- chlorophenyl) propyl]-7- cyclopentyl- benzo[g] pteridine- 2,4(3H,10H)- dione |
| 92 | 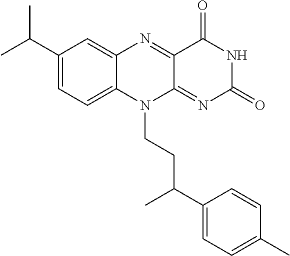 [SMILES: Cc1ccc(C(C)CCn2c3nc(=O)[nH]c(=O)c-3nc3cc(C(C)C)ccc32)cc1] | 403.1 | 4.24 | D | Prepared by the synthesis of example 1 starting from 4- isopropyl-2- nitroaniline and 1-(3- bromo-1- methylpropyl)-4- methylbenzene | 7-isopropyl- 10-[3-(4- methylphenyl) butyl]benzo [g]pteridine- 2,4(3H,10H)- dione |
| 93 | 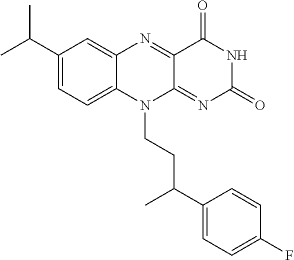 [SMILES: CC(C)c1ccc2c(c1)nc1c(=O)[nH]c(=O)nc-1n2CCC(C)c1ccc(F)cc1] | 407.1 | 4.06 | D | Prepared by the synthesis of example 1 starting from 4- isopropyl-2- nitroaniline and 1-(3- bromo-1- methylpropyl)-4- fluorobenzene | 10-[3-(4- fluorophenyl) butyl]-7- isopropylbenzo [g]pteridine- 2,4(3H,10H)- dione |
| 94 | 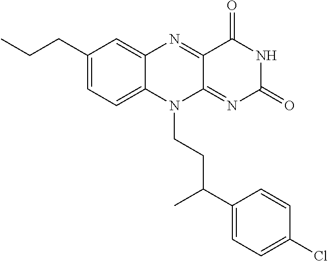 [SMILES: CCCc1ccc2c(c1)nc1c(=O)[nH]c(=O)nc-1n2CCC(C)c1ccc(Cl)cc1] | 423.1 | 4.33 | D | Prepared by the synthesis of example 1 starting from 4- propyl-2-nitroaniline and 1-(3-bromo-1- methylpropyl)-4- chlorobenzene | 10-[3-(4- chlorophenyl) butyl]-7- propylbenzo [g]pteridine- 2,4(3H,10H)- dione |
| 95 | 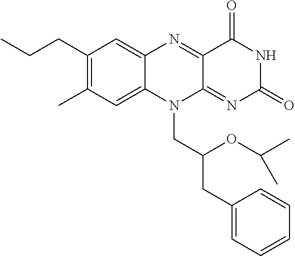 [SMILES: CCCc1cc2nc3c(=O)[nH]c(=O)nc-3n(CC(Cc3ccccc3)OC(C)C)c2cc1C] | 447.3 | 4.48 | D | Prepared by the synthesis of example 6 starting from 4- propyl-2-nitroaniline and (3-amino-2- isopropoxypropyl) benzene | 10-(2- isopropoxy- 3- phenylpropyl)- 8-methyl-7- propylbenzo [g]pteridine- 2,4(3H,10H)- dione |
| 96 | 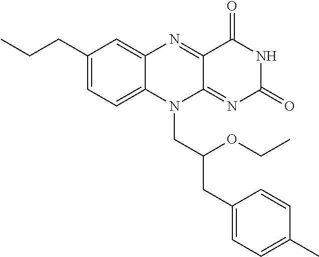 [SMILES: CCCc1ccc2c(c1)nc1c(=O)[nH]c(=O)nc-1n2CC(Cc1ccc(C)cc1)OCC] | 433.1 | 4.71 | G | Prepared by the synthesis of example 1 starting from 4- propyl-2-nitroaniline and 1-(3-Bromo-2- ethoxypropyl)-4- methylbenzene | 10-[2-ethoxy- 3-(4- methylphenyl) propyl]-7- propylbenzo [g]pteridine- 2,4(3H,10H)- dione |
| 97 | 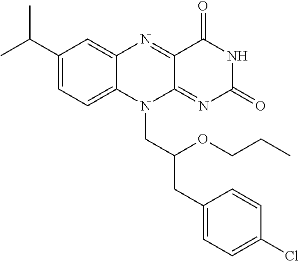 [SMILES: CCCOC(Cc1ccc(Cl)cc1)Cn1c2nc(=O)[nH]c(=O)c-2nc2cc(C(C)C)ccc21] | 467.1 | 4.65 | D | Prepared by the synthesis of example 1 starting from 4- isopropyl-2- nitroaniline and 1-(3- bromo-2- propoxypropyl)-4- chlorobenzene | 10-[3-(4- chlorophenyl)- 2- propoxypropyl]- 7- isopropylbenzo [g]pteridine- 2,4(3H,10H)- dione |
| 98 | 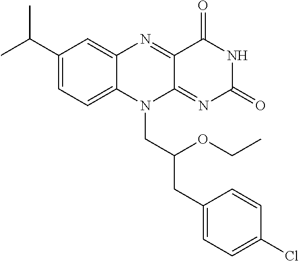 [SMILES: CCOC(Cc1ccc(Cl)cc1)Cn1c2nc(=O)[nH]c(=O)c-2nc2cc(C(C)C)ccc21] | 453.0 | 4.73 | G | Prepared by the synthesis of example 6 starting from 1- bromo-4-isopropyl-2- nitro-benzene and 3- (4-chlorophenyl)-2- ethoxypropan-1- amine | 10-[3-(4- chlorophenyl)- 2- ethoxypropyl]- 7- isopropylbenzo [g]pteridine- 2,4(3H,10H)- dione |
| 99 | 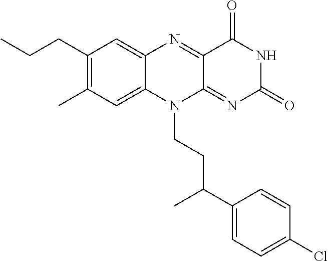 [SMILES: CCCc1cc2nc3c(=O)[nH]c(=O)nc-3n(CCC(C)c3ccc(Cl)cc3)c2cc1C] | 437.1 | 4.51 | D | Prepared by the synthesis of example 1 starting from 4-ethyl- 5-methyl-2- nitroaniline and 1-(3- bromo-1- methylpropyl)-4- chlorobenzene | 10-[3-(4- chlorophenyl) butyl]-8- methyl-7- propylbenzo [g]pteridine- 2,4(3H,10H)- dione |
| 100 | 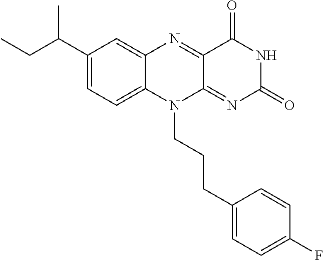 [SMILES: CCC(C)c1ccc2c(c1)nc1c(=O)[nH]c(=O)nc-1n2CCCc1ccc(F)cc1] | 407.1 | 4.11 | D | Prepared by the synthesis of example 1 starting from 4-sec- butyl-2-nitroaniline and 1-(3- bromopropyl)-4- fluorobenzene | 7-sec-butyl- 10-[3-(4- fluorophenyl) propyl]benzo [g]pteridine- 2,4(3H,10H)- dione |
| 101 | 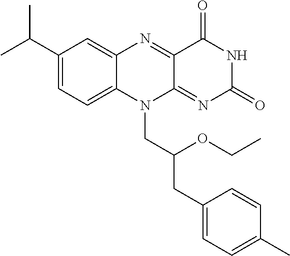 [SMILES: CCOC(Cc1ccc(C)cc1)Cn1c2nc(=O)[nH]c(=O)c-2nc2cc(C(C)C)ccc21] | 433.1 | 4.67 | G | Prepared by the synthesis of example 1 starting from 4- isopropyl-2- nitroaniline and 1-(3- Bromo-2- ethoxypropyl)-4- methylbenzene | 10-[2-ethoxy- 3-(4- methylphenyl) propyl]-7- isopropylbenzo [g]pteridine- 2,4(3H,10H)- dione |
| 102 | 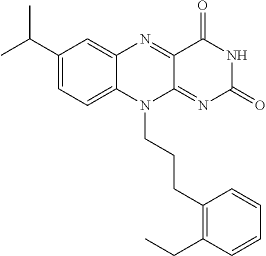 [SMILES: CCc1ccccc1CCCn1c2nc(=O)[nH]c(=O)c-2nc2cc(C(C)C)ccc21] | 403.1 | 4.26 | D | Prepared by the synthesis of example 1 starting from 4- isopropyl-2- nitroaniline and 1-(3- bromopropyl)-2- ethylbenzene | 10-[3-(2- ethylphenyl) propyl]-7- isopropylbenzo [g]pteridine- 2,4(3H,10H)- dione |
| 103 | 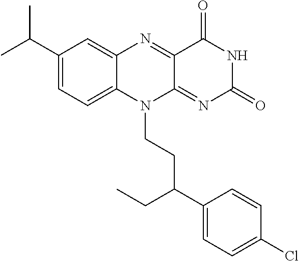 [SMILES: CCC(CCn1c2nc(=O)[nH]c(=O)c-2nc2cc(C(C)C)ccc21)c1ccc(Cl)cc1] | 437.2 | 4.88 | G | Prepared by the synthesis of example 1 starting from 4- isopropyl-2- nitroaniline and 1-(3- bromo-1-ethyl- propyl)-4-chloro- benzene | 10-[3-(4- chlorophenyl) pentyl]-7- isopropylbenzo [g]pteridine- 2,4(3H,10H)- dione |
| 104 | 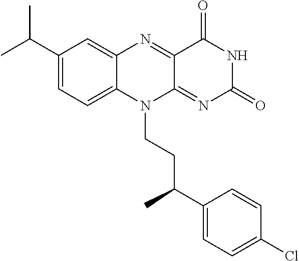 [SMILES: CC(C)c1ccc2c(c1)nc1c(=O)[nH]c(=O)nc-1n2CC[C@H](C)c1ccc(Cl)cc1] | 423.1 | 4.29 | D | Prepared by the synthesis of example 1 starting from 4- isopropyl-2- nitroaniline and 1- [(1S)-3-bromo-1- methylpropyl]-4- chlorobenzene | 10-[(3S)-3-(4- chlorophenyl) butyl]-7- isopropylbenzo [g]pteridine- 2,4(3H,10H)- dione |
| 105 | 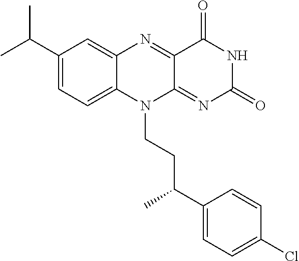 [SMILES: CC(C)c1ccc2c(c1)nc1c(=O)[nH]c(=O)nc-1n2CC[C@@H](C)c1ccc(Cl)cc1] | 423.0 | 4.29 | D | Prepared by the synthesis of example 1 starting from 4- isopropyl-2- nitroaniline and 1- [(1R)-3-bromo-1- methylpropyl]-4- chlorobenzene | 10-[(3R)-3-(4- chlorophenyl) butyl]-7- isopropylbenzo [g]pteridine- 2,4(3H,10H)- dione |
| 106 | 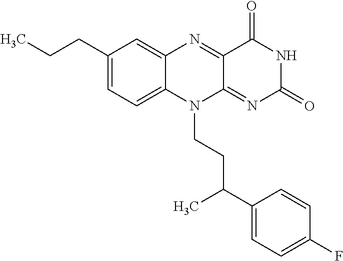 [SMILES: CCCc1ccc2c(c1)nc1c(=O)[nH]c(=O)nc-1n2CCC(C)c1ccc(F)cc1] | 407.1 | 4.08 | D | Prepared by the synthesis of example 1 starting from 4- propyl-2-nitroaniline and 1-(3-bromo-1- methylpropyl)-4- fluorobenzene | 10-[3-(4- fluorophenyl) butyl]-7- propylbenzo [g]pteridine- 2,4(3H,10H)- dione |

1. A compound of Formula P: 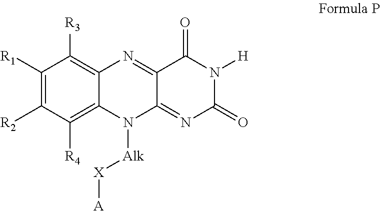
wherein:
(i) Alk is C₁₋₆alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl, ethyl or isobutyl), arylC₁₋₄alkyl (e.g., benzyl) and/or —N(R_{c})(R_{d}); or
Alk is C₁₋₆alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄alkoxy (e.g., methoxy, ethoxy, propoxy, isobutoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
C₁₋₄alkyl (e.g., methyl, ethyl, t-butyl or n-prop-2-en-1-yl),
C₁₋₄alkoxy (e.g., methoxy),
hydroxy,
—O—C₁₋₄alkyl-N(R_{c})(R_{d}), for example —OCH₂CH₂N(CH₃)₂,
halo (e.g., Cl, F),
haloC₁₋₄alkyl (e.g., CF₃),
—O-haloC₁₋₄alkyl (e.g., —OCF₃),
cyano,
—O—(CH₂CH₂O)₁₋₃—C₁₋₄alkyl (e.g., —OCH₂CH₂OCH₃ or —O(CH₂CH₂O)₃CH₃), and/or
—CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
H,
C₁₋₆alkyl, e.g., C₁₋₄alkyl (for example, methyl, ethyl, n-propyl, isopropyl, isobutyl, t-butyl, 1-methylpropyl or n-hexyl),
C₃₋₈cycloalkyl (e.g., cyclopropyl or cyclopentyl),
aryl (e.g., phenyl), or
C₁₋₄alkoxy (e.g., methoxy);
(v) R₂ is:
H,
C₁₋₆alkyl, C₁₋₄alkyl (for example, methyl, ethyl, n-propyl, isopropyl, n-prop-2-en-1-yl, n-butyl, isobutyl, n-but-2-en-1-yl, n-hexyl),
—C₀₋₄alkyl-C₃₋₈cycloalkyl (e.g., cyclopropyl),
—C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
—C₀₋₄alkyl-N(Rₐ)(R_{b}), for example —C₀alkyl-N(Rₐ)(R_{b}) or —C₁alkyl-N(Rₐ)(R_{b}),
C₁₋₄alkoxy (e.g., methoxy),
halo (e.g., Cl),
—O—(CH₂CH₂O)₁₋₃—C₁₋₄alkyl (e.g., —OCH₂CH₂OCH₃ or —O(CH₂CH₂O)₃CH₃),
—N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
—N(Rₑ)—C(O)—O—C₁₋₄alkyl (e.g., —N(H)—C(O)—O—C(H)(CH₃)CH₃),
—N(Rₑ)—C(O)-aryl wherein said aryl is optionally substituted with one or more halo (e.g., F), for example —N(H)—C(O)-(4-fluorophenyl),
—C₁₋₆alkyl-OC₁₋₄alkyl (e.g., —CH₂CH₂CH₂CH₂—O—CH₃),
—O—CH₂CH₂—O—CH₂-phenyl,
—O-haloC₁₋₄alkyl (e.g., —OCH₂CF₃),
—CH₂—O—C(O)—C₁₋₄alkyl (e.g., —CH₂—O—C(O)—CH₃),
—C(O)O—C₁₋₄alkyl (e.g., —C(O)OCH₃), or
C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
H,
C₁₋₄alkyl (e.g., methyl) optionally substituted with one or more hydroxy groups for example, 2,3-dihydroxyprop-1-yl,
C₃₋₈cycloalkyl (e.g., cyclopropyl or cyclopentyl),
C₁₋₄alkoxy-C₁₋₄alkyl (e.g., methoxyethyl),
hydroxy-C₁₋₄alkyl (e.g., hydroxyethyl),
N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
in free or salt form, provided that:
(1) when -Alk-X-A is —CH₂CH₂-phenyl or —CH₂CH₂—O-phenyl, R₁ and R₂ are not both H;
(2) when -Alk-X-A is —CH₂CH₂-(3-methoxyphenyl), R₁ and R₂ are not both methyl; or
(3) when R₂ is —C(O)OEt and -Alk-X-A is phenylethyl, then R₁ is C₁₋₆alkyl, e.g., C₁₋₄alkyl (for example, methyl, ethyl, n-propyl, isopropyl, n-prop-2-enyl, n-butyl, n-but-2-en-yl or n-hexyl), C₃₋₈cycloalkyl (e.g., cyclopropyl), or C₁₋₄alkoxy (e.g., methoxy).

2. The compound according to claim 1, wherein said compound is a compound of formula Q 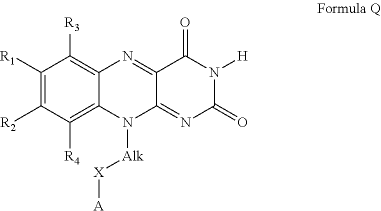
wherein:
(i) Alk is C₁₋₆alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl or isobutyl) and/or —N(R_{c})(R_{d}); or
Alk is C₁₋₆alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄alkoxy (e.g., methoxy, ethoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
C₁₋₄alkyl (e.g., methyl, t-butyl or n-prop-2-en-1-yl),
C₁₋₄alkoxy (e.g., methoxy),
hydroxy,
—O—C₁₋₄alkyl-N(R_{c})(R_{d}), for example —OCH₂CH₂N(CH₃)₂,
halo (e.g., Cl, F),
haloC₁₋₄alkyl (e.g., CF₃),
—O-haloC₁₋₄alkyl (e.g., —OCF₃),
cyano,
—O—(CH₂CH₂O)₁₋₃—C₁₋₄alkyl (e.g., —OCH₂CH₂OCH₃ or —O(CH₂CH₂O)₃CH₃), and/or
—CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
H,
C₁₋₆alkyl, e.g., C₁₋₄alkyl (for example, methyl, ethyl, n-propyl, isopropyl, isobutyl, t-butyl, 1-methylpropyl or n-hexyl),
C₃₋₈cycloalkyl (e.g., cyclopropyl),
aryl (e.g., phenyl), or
C₁₋₄alkoxy (e.g., methoxy);
(v) R₂ is:
H,
C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
—C₀₋₄alkyl-C₃₋₈cycloalkyl (e.g., cyclopropyl),
—C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
—C₀₋₄alkyl-N(Rₐ)(R_{b}), for example —C₀alkyl-N(Rₐ)(R_{b}) or —C₁alkyl-N(Rₐ)(R_{b}),
C₁₋₄alkoxy (e.g., methoxy),
halo (e.g., Cl),
—O—(CH₂CH₂O)₁₋₃—C₁₋₄alkyl (e.g., —OCH₂CH₂OCH₃ or —O(CH₂CH₂O)₃CH₃),
—N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
—N(Rₑ)—C(O)—O—C₁₋₄alkyl (e.g., —N(H)—C(O)—O—C(H)(CH₃)CH₃),
—N(Rₑ)—C(O)-aryl wherein said aryl is optionally substituted with one or more halo (e.g., F), for example —N(H)—C(O)-(4-fluorophenyl),
—C₁₋₆alkyl-OC₁₋₄alkyl (e.g., —CH₂CH₂CH₂CH₂—O—CH₃),
—O—CH₂CH₂—O—CH₂-phenyl,
—O-haloC₁₋₄alkyl (e.g., —OCH₂CF₃),
—CH₂—O—C(O)—C₁₋₄alkyl (e.g., —CH₂—O—C(O)—CH₃),
—C(O)O—C₁₋₄alkyl (e.g., —C(O)OCH₃), or
C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
H,
C₁₋₄alkyl (e.g., methyl) optionally substituted with one or more hydroxy groups for example, 2,3-dihydroxyprop-1-yl,
C₃₋₈cycloalkyl (e.g., cyclopropyl or cyclopentyl),
C₁₋₄alkoxy-C₁₋₄alkyl (e.g., methoxyethyl),
hydroxy-C₁₋₄alkyl (e.g., hydroxyethyl),
N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
in free or salt form.

3. The compound according to claim 1 or 2, wherein said compound is a compound of Formula I: 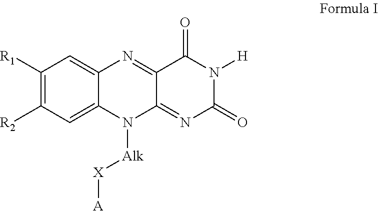
wherein:
(i) Alk is C₁₋₆alkylene (e.g., ethylene, n-propylene, n-butylene, n-pentylene) optionally substituted with one or more C₁₋₄alkyl, —N(R_{c})(R_{d}); or
Alk is C₁₋₆alkylene (e.g., n-propylene, n-butylene, n-pentylene) optionally substituted with one hydroxy or C₁₋₄alkoxy group;
(ii) X is a single bond, —S— or —O—;
(iii) A is aryl (e.g., phenyl) or aryl-C₁₋₄alkyl (e.g., benzyl), wherein the aryl group of said aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl) or C₁₋₄alkoxy (e.g., methoxy);
(v) R₂ is H, C₁₋₄alkyl (e.g., methyl), —C₀₋₄alkyl-C₃₋₈cycloalkyl (e.g., cyclopropyl), —C₀₋₄alkyl-N(Rₐ)(R_{b}), C₁ alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy; or
(vi) Optionally, R₁ and R₂ are linked together so that together with the carbon atoms to which they are attached they form a cyclic structure (e.g., R₁ and R₂ are linked together to form a ethylenedioxy);
(vii) Rₐ and R_{b} are independently H, C₁₋₄alkyl (e.g., methyl), C₃₋₈cycloalkyl (e.g., cyclopropyl, cyclopentyl), C₁₋₄alkoxy-C₁₋₄alkyl (e.g., methoxyethyl), hydroxy-C₁₋₄alkyl (e.g., hydroxyethyl), N(R_{c})(R_{d})—C₁₋₄alkyl (e.g., dimethylaminoethyl);
(viii) R_{c} and R_{d} are independently H or C₁₋₄alkyl (e.g., methyl);
in free or salt form, provided that (1) when -Alk-X-A is —CH₂CH₂-phenyl or —CH₂CH₂—O-phenyl, R₁ and R₂ are not both H; or (2) when -Alk-X-A is —CH₂CH₂-(3-methoxyphenyl), —CH₂CH₂-(3,4,5-trimethoxyphenyl), —CH₂CH₂CH₂-(2,5-dimethoxyphenyl) or —CH₂CH₂CH₂-(2,5-dihydroxyphenyl), R₁ and R₂ are not both methyl.

4. The compound according to any one of claims 1-3, wherein:
Alk is C₂₋₃alkylene (e.g., ethylene, i.e., CH₂CH₂—, n-propylene, i.e., —CH₂CH₂CH₂—) optionally substituted with one or more C₁₋₄alkyl (e.g., methyl, ethyl or isobutyl); or
Alk is C₂₋₃alkylene (e.g., ethylene, i.e., CH₂CH₂— or n-propylene, i.e., —CH₂CH₂CH₂—) optionally substituted with one C₁₋₄alkoxy (e.g., ethoxy or isopropyloxy) group;
X is a single bond, —S— or —O—;
A is aryl (e.g., phenyl), wherein the aryl group is optionally substituted with one or more
C₁₋₄alkyl (e.g., methyl),
halo (e.g., Cl, F),
R₁ is:
C₁₋₆alkyl, e.g., C₁₋₄alkyl (for example, methyl, ethyl, n-propyl, isopropyl, isobutyl, t-butyl, 1-methylpropyl),
C₃₋₈cycloalkyl (e.g., cyclopentyl),
R₂ is:
H,
C₁₋₆alkyl, e.g., C₁₋₄alkyl (for example, methyl, ethyl, n-propyl or isopropyl),
—C₀₋₄alkyl-C₃₋₈cycloalkyl (e.g., cyclopropyl),
in free or salt form.

5. The compound according to any one of claims 1-4, wherein:
Alk is C₃alkylene (e.g., n-propylene, i.e., —CH₂CH₂CH₂—) optionally substituted with one or more C₁₋₄alkyl (e.g., methyl or ethyl); or
Alk is C₃alkylene (e.g., n-propylene, i.e., —CH₂CH₂CH₂—) optionally substituted with one C₁₋₄alkoxy (e.g., ethoxy or isopropyloxy) group;
X is a single bond;
A is aryl (e.g., phenyl), wherein the aryl group is optionally substituted with one or more
C₁₋₄alkyl (e.g., methyl),
halo (e.g., Cl, F),
R₁ is:
C₁₋₆alkyl, e.g., C₁₋₄alkyl (for example, methyl, ethyl, n-propyl, isopropyl or 1-methylpropyl),
C₃₋₈cycloalkyl (e.g., cyclopentyl),
R₂ is:
H,
C₁₋₆alkyl, e.g., C₁₋₄alkyl (for example, methyl, ethyl, n-propyl or isopropyl),
—C₀₋₄alkyl-C₃₋₈cycloalkyl (e.g., cyclopropyl),
in free or salt form.

6. The compound according to any one of claims 1-5, wherein:
Alk is —CH₂CH₂CH₂—;
X is a single bond;
A is aryl (e.g., phenyl);
R₁ is C₁₋₆alkyl, e.g., C₁₋₄alkyl (for example, methyl),
R₂ is C₁₋₆alkyl, e.g., C₁₋₄alkyl (for example, methyl),
R₃ and R₄ are H;
in free or salt form.

7. The compound according to any one of claims 1-6 selected from any of following: 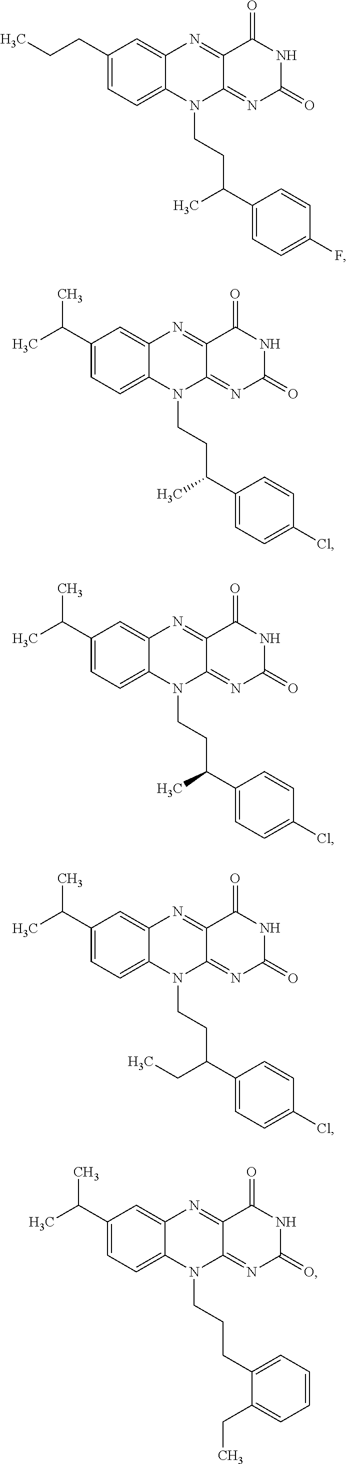 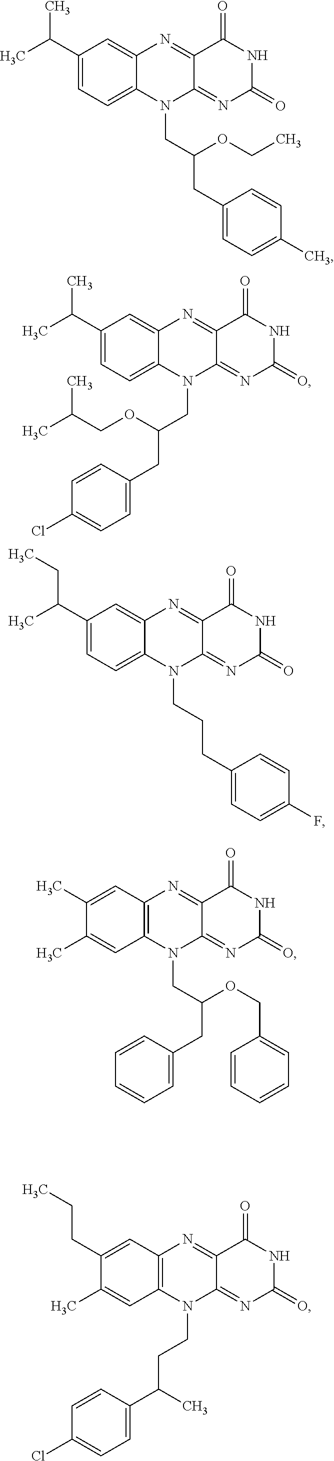 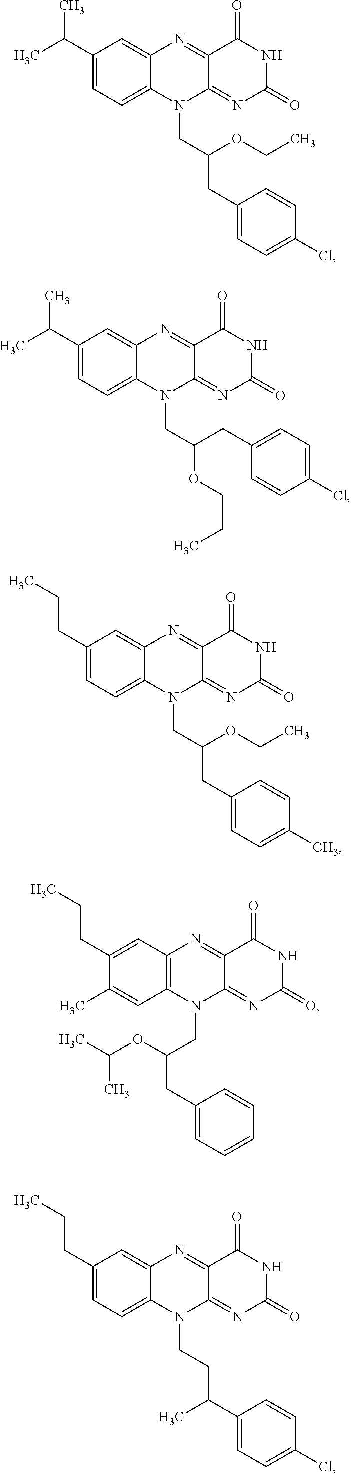 [SMILES: CCOC(Cc1ccc(Cl)cc1)Cn1c2nc(=O)[nH]c(=O)c-2nc2cc(C(C)C)ccc21 ; CCCOC(Cc1ccc(Cl)cc1)Cn1c2nc(=O)[nH]c(=O)c-2nc2cc(C(C)C)ccc21 ; CCCc1ccc2c(c1)nc1c(=O)[nH]c(=O)nc-1n2CC(Cc1ccc(C)cc1)OCC ; CCCc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCC(C)c1ccc(Cl)cc1] 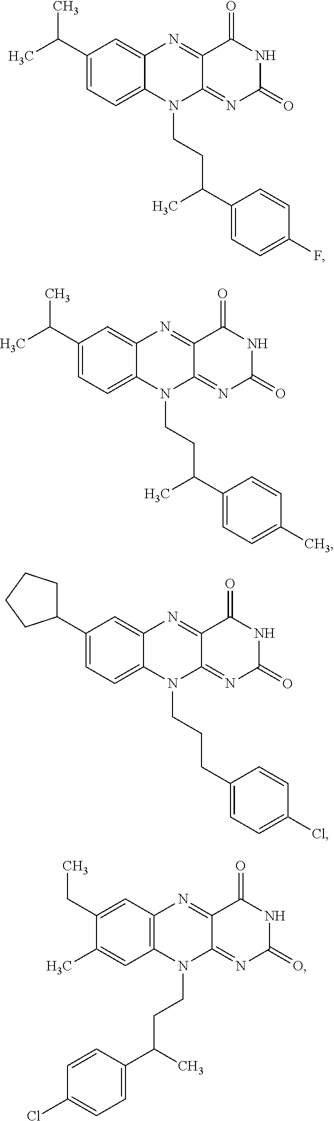 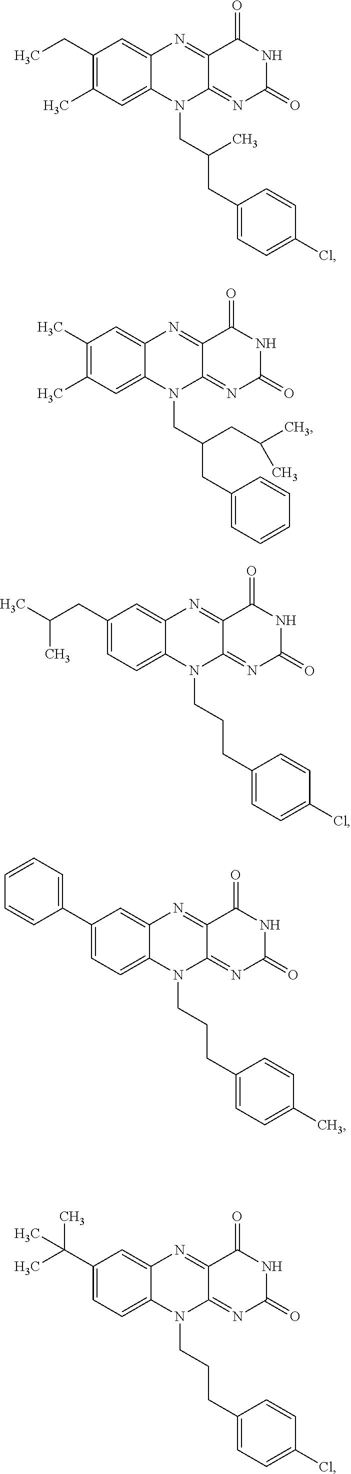 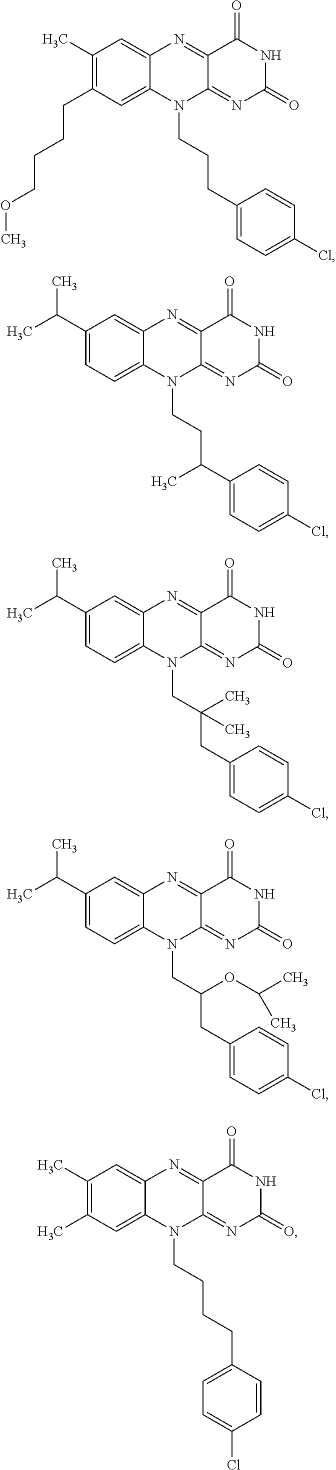 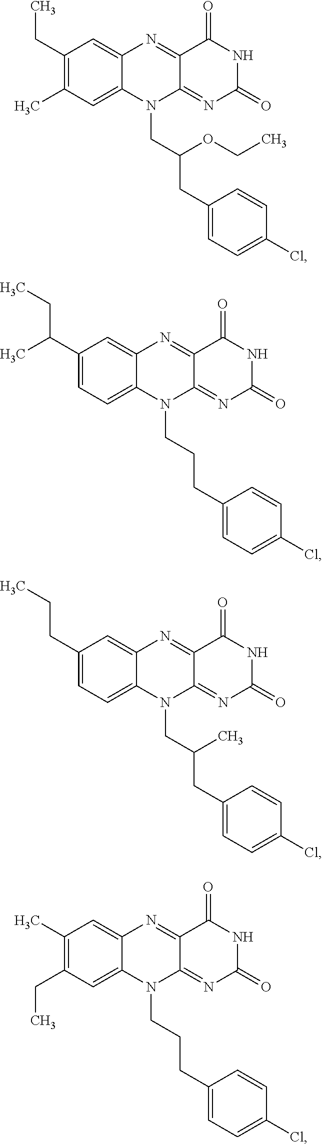 [SMILES: CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1 ; CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1] 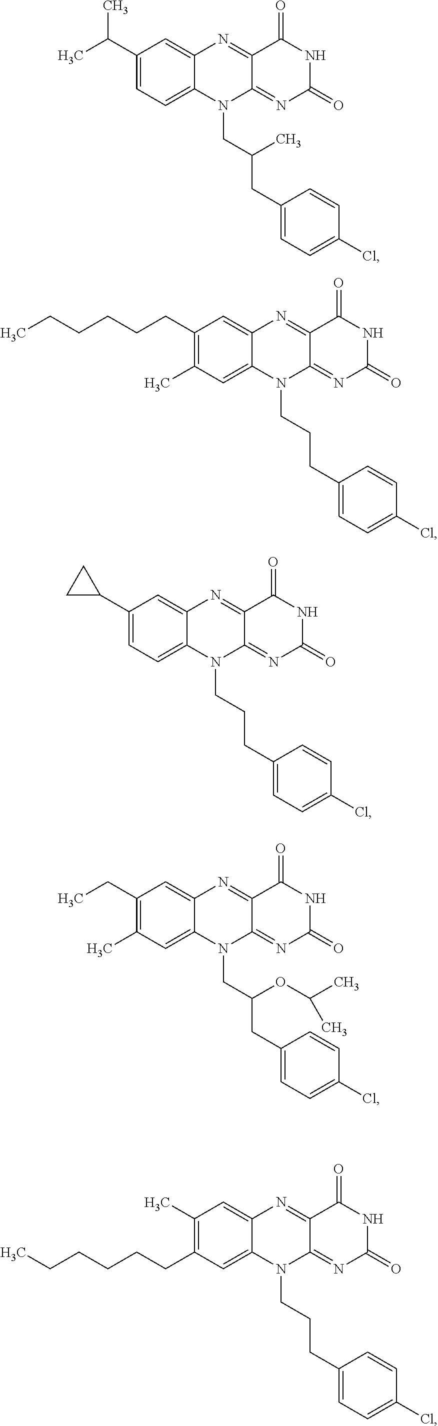 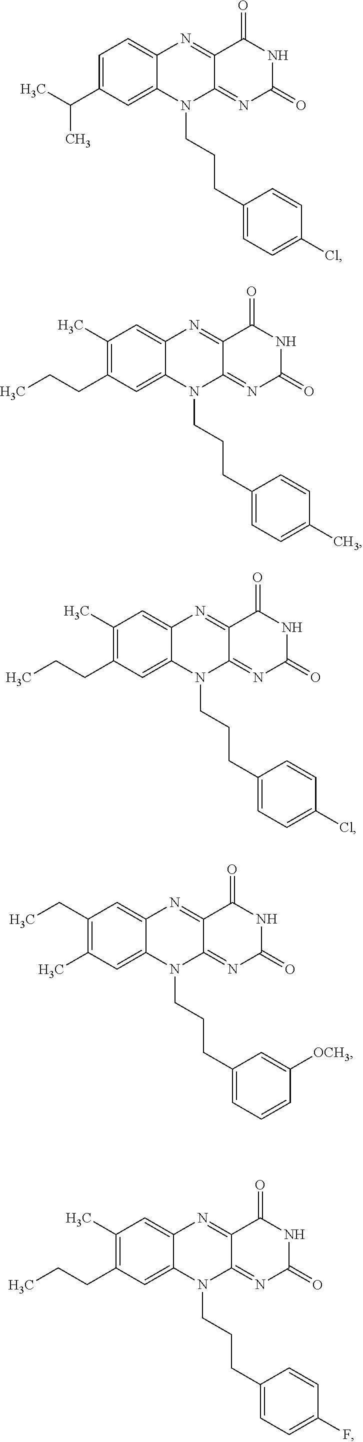 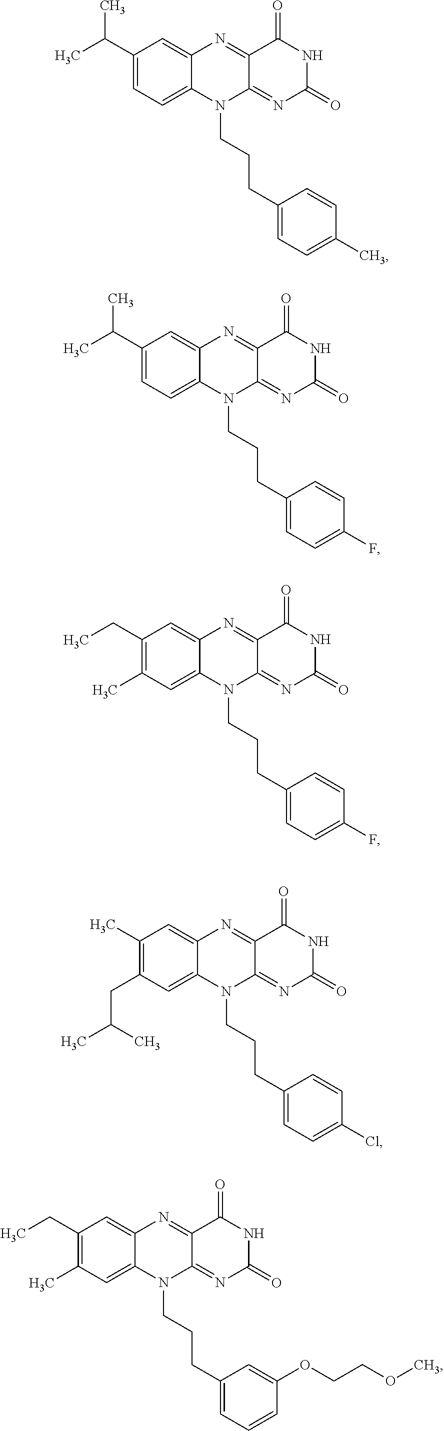 [SMILES: Cc1ccc(CCCn2c3nc(=O)[nH]c(=O)c-3nc3cc(C(C)C)ccc32)cc1 ; CC(C)c1ccc2c(c1)nc1c(=O)[nH]c(=O)nc-1n2CCCc1ccc(F)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C ; CCc1cc2nc3c(=O)[nH]c(=O)nc-3n(CCCc3cccc(OCCOC)c3)c2cc1C] 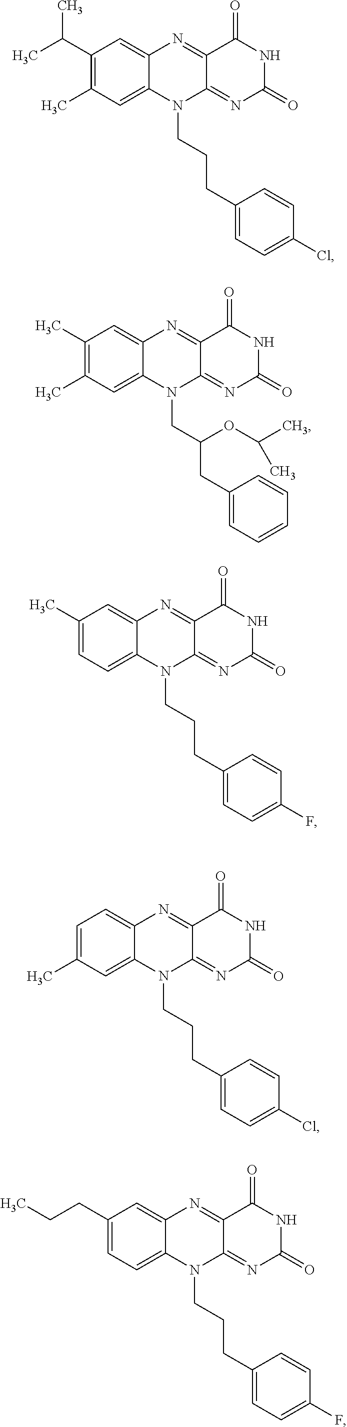 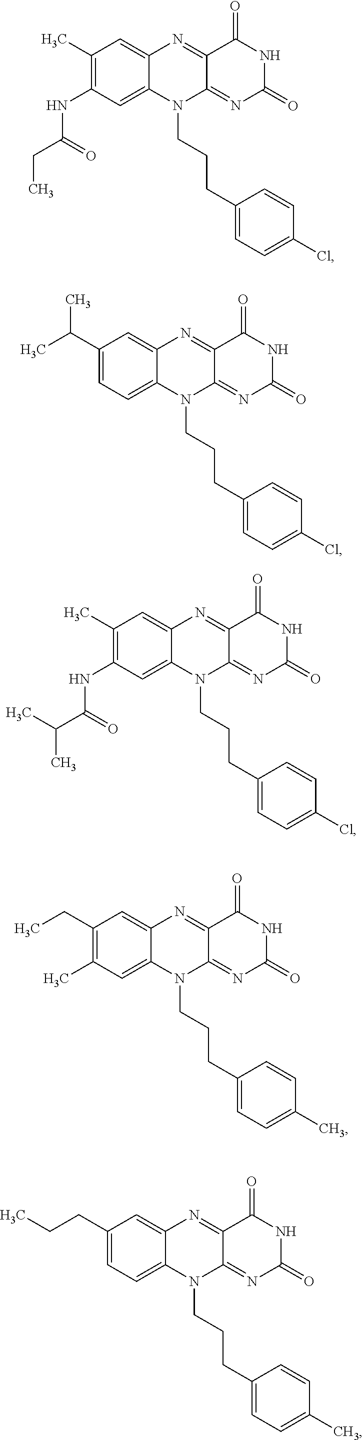 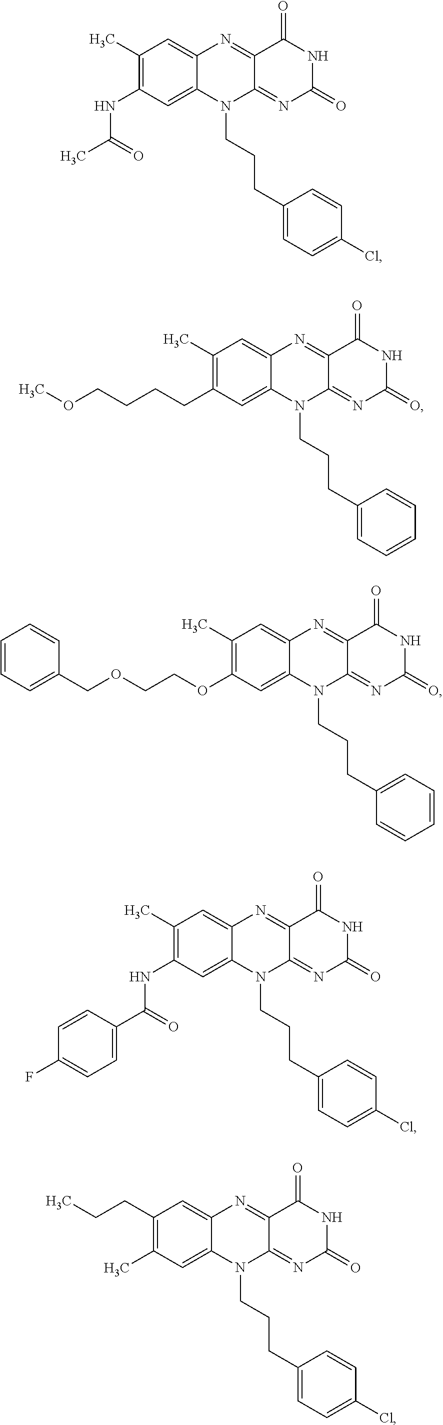 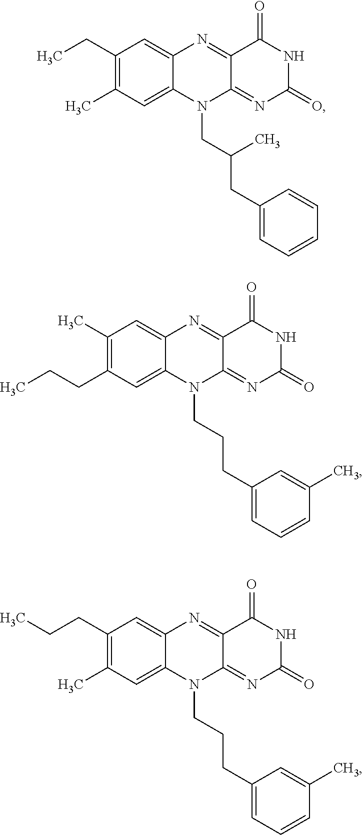 [SMILES: CCc1cc2nc3c(=O)[nH]c(=O)nc-3n(CC(C)Cc3ccccc3)c2cc1C ; CCCc1cc2c(cc1C)nc1c(=O)[nH]c(=O)nc-1n2CCCc1cccc(C)c1 ; CCCc1cc2nc3c(=O)[nH]c(=O)nc-3n(CCCc3cccc(C)c3)c2cc1C] 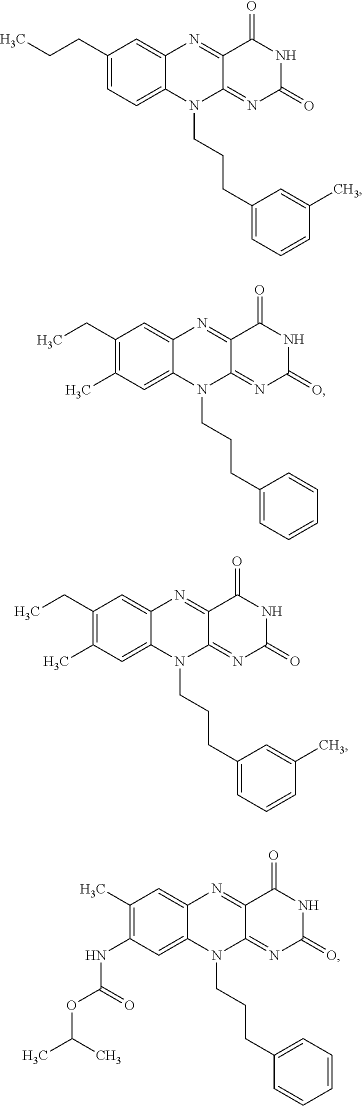 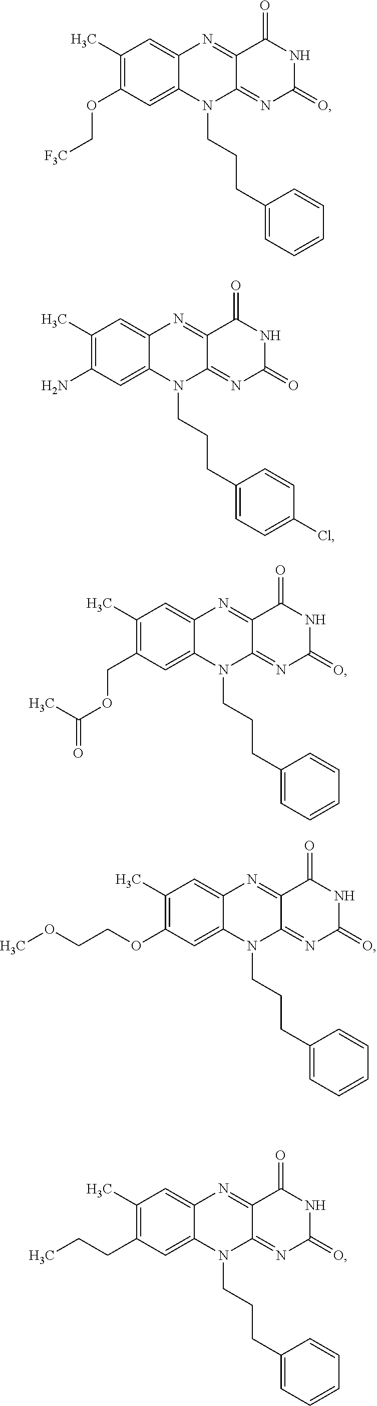 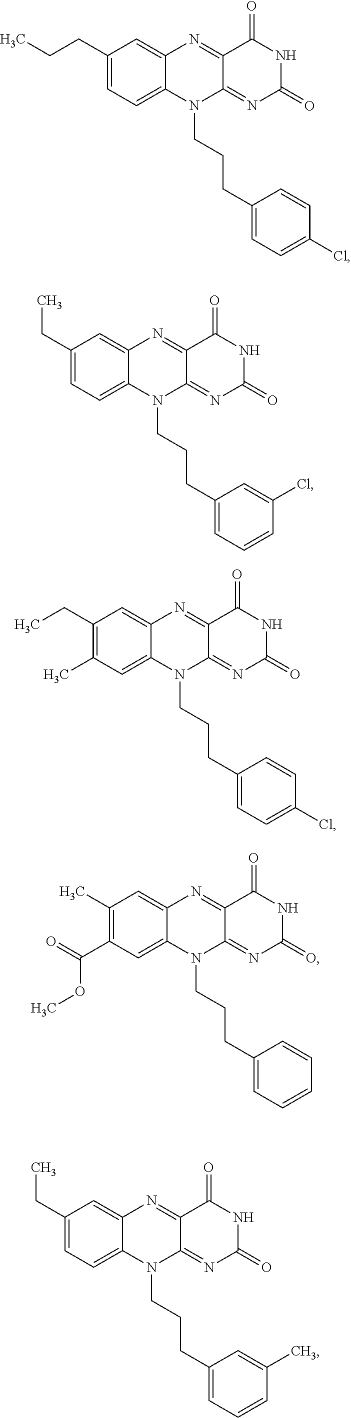 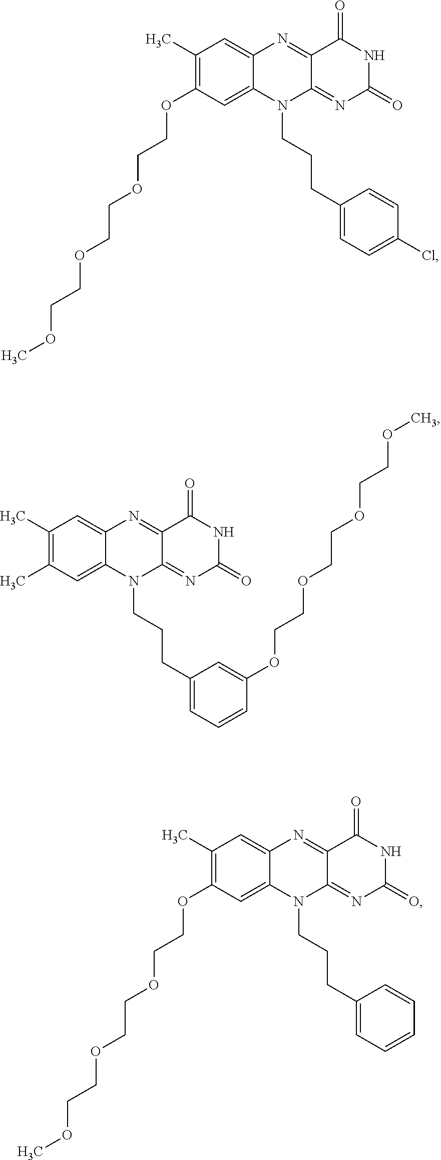 [SMILES: COCCOCCOCCOc1cc2c(cc1C)nc1c(=O)[nH]c(=O)nc-1n2CCCc1ccc(Cl)cc1 ; COCCOCCOCCOc1cccc(CCCn2c3nc(=O)[nH]c(=O)c-3nc3cc(C)c(C)cc32)c1 ; COCCOCCOCCOc1cc2c(cc1C)nc1c(=O)[nH]c(=O)nc-1n2CCCc1ccccc1] 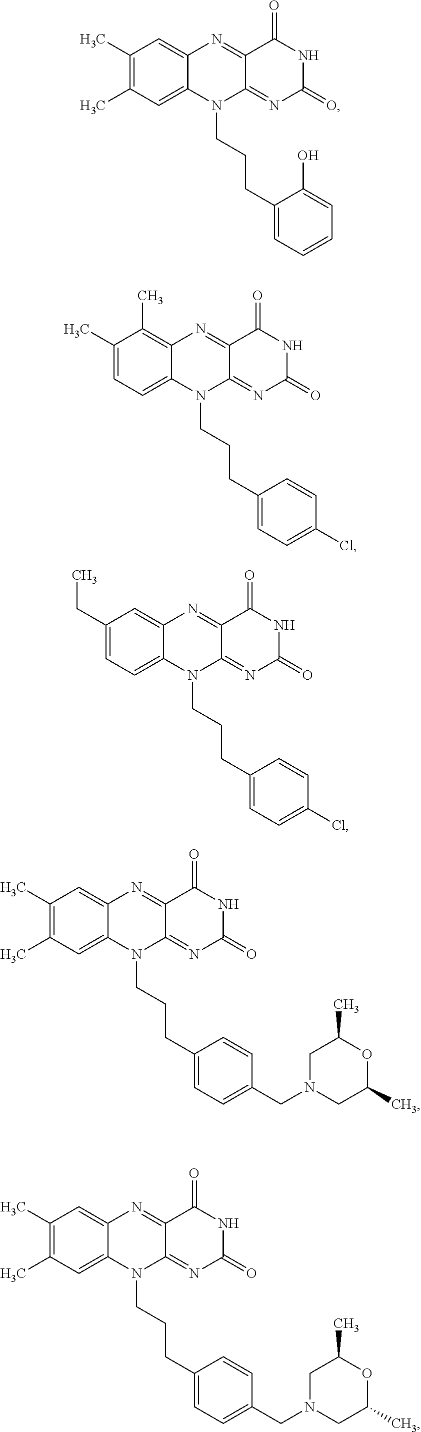 [SMILES: Cc1cc2nc3c(=O)[nH]c(=O)nc-3n(CCCc3ccccc3O)c2cc1C ; Cc1ccc2c(nc3c(=O)[nH]c(=O)nc-3n2CCCc2ccc(Cl)cc2)c1C ; CCc1ccc2c(c1)nc1c(=O)[nH]c(=O)nc-1n2CCCc1ccc(Cl)cc1 ; Cc1cc2nc3c(=O)[nH]c(=O)nc-3n(CCCc3ccc(CN4C[C@@H](C)O[C@@H](C)C4)cc3)c2cc1C ; Cc1cc2nc3c(=O)[nH]c(=O)nc-3n(CCCc3ccc(CN4C[C@@H](C)O[C@H](C)C4)cc3)c2cc1C] 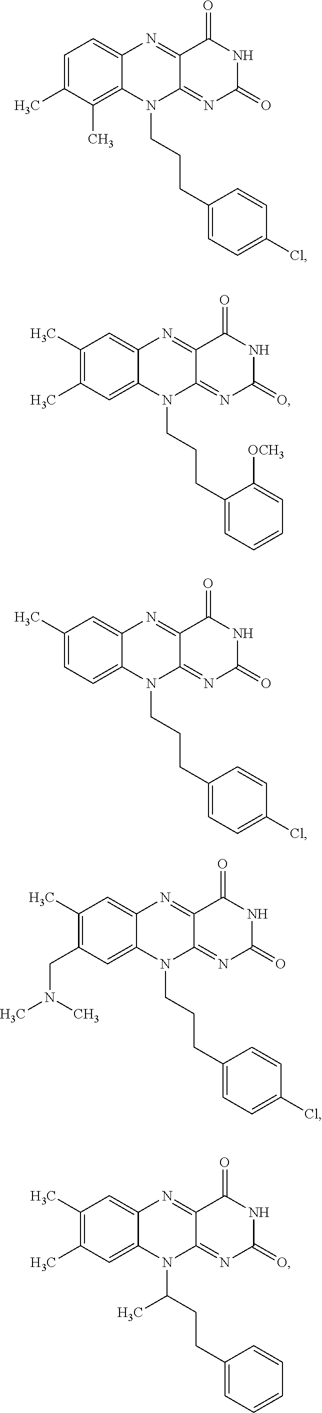 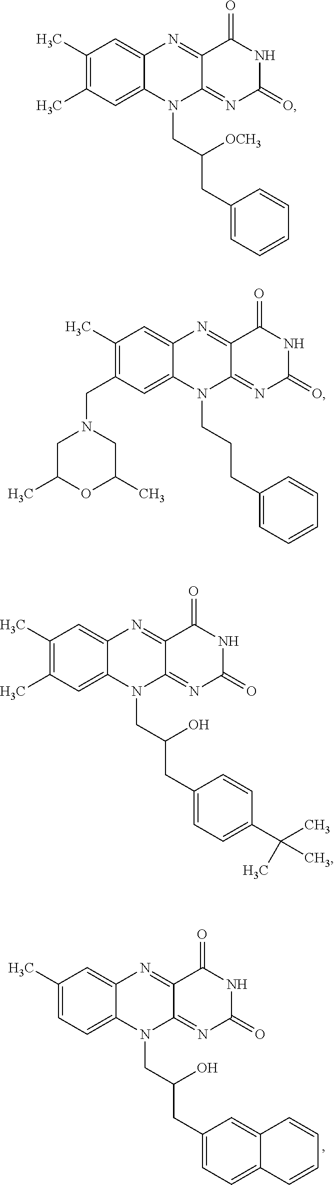 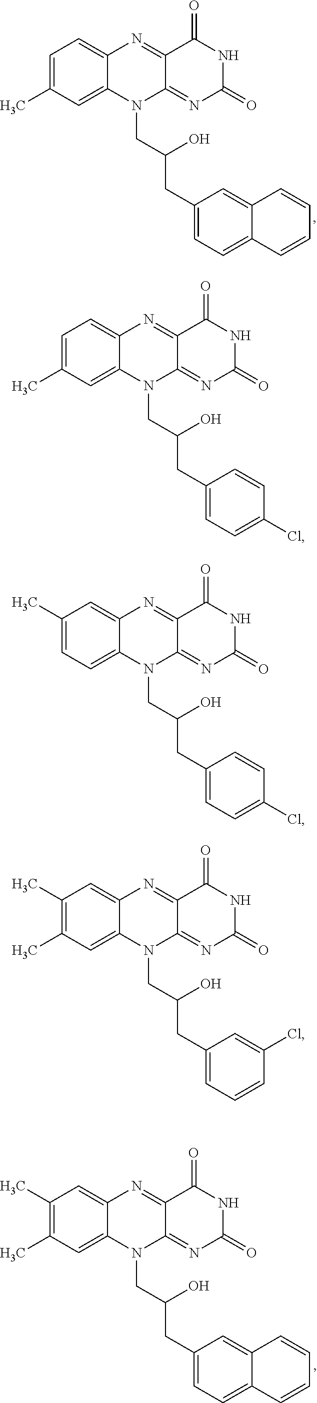 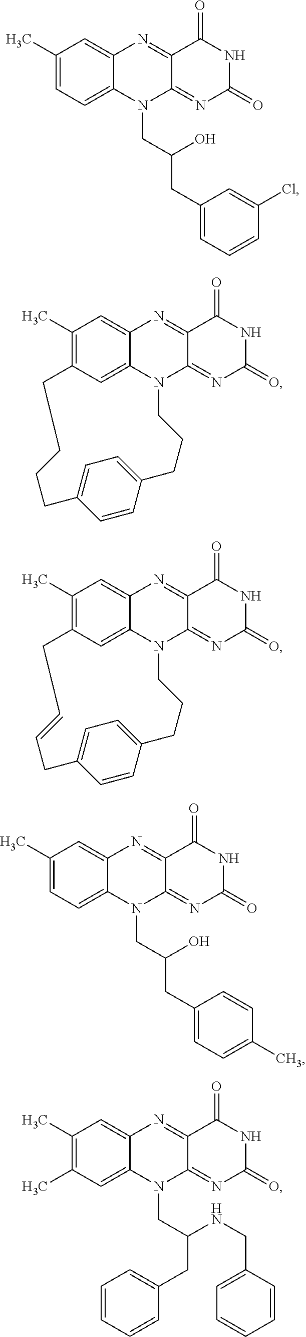 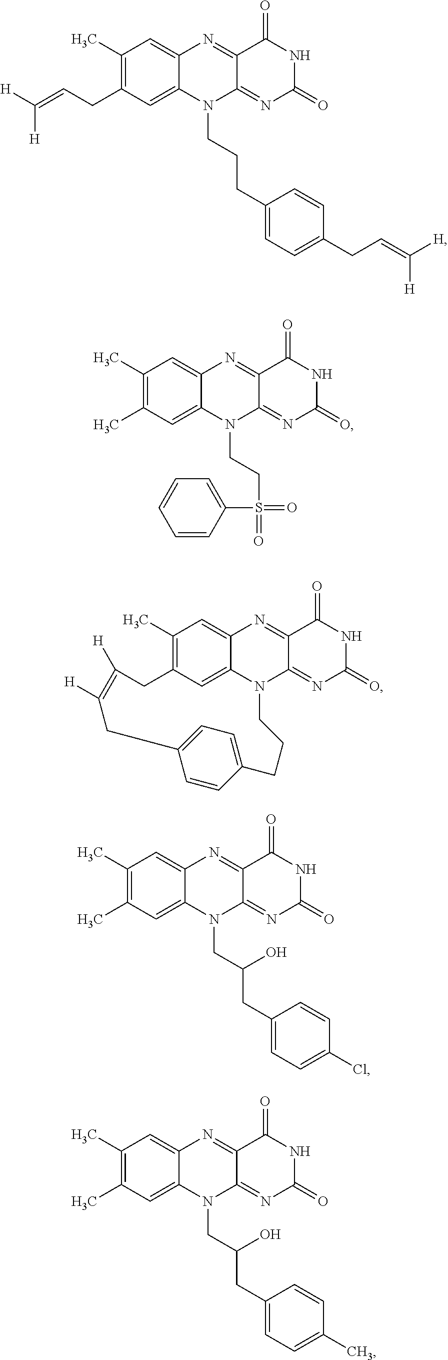 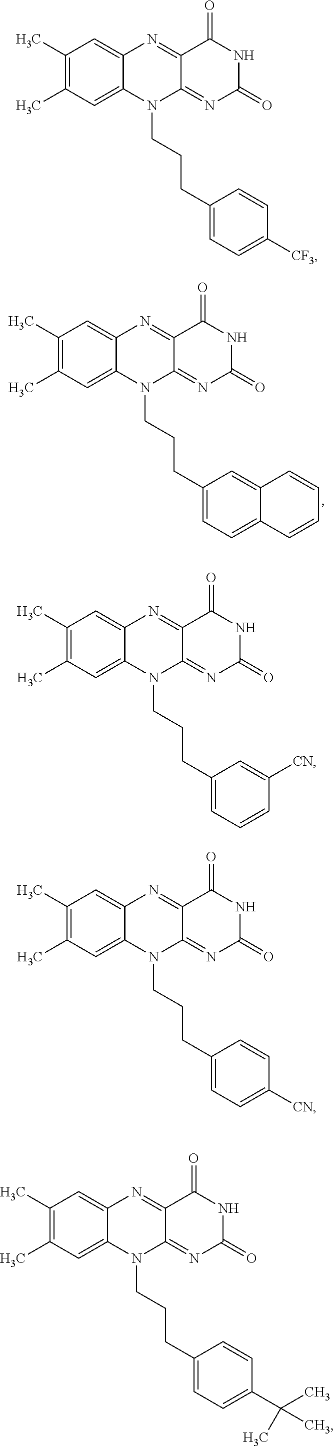 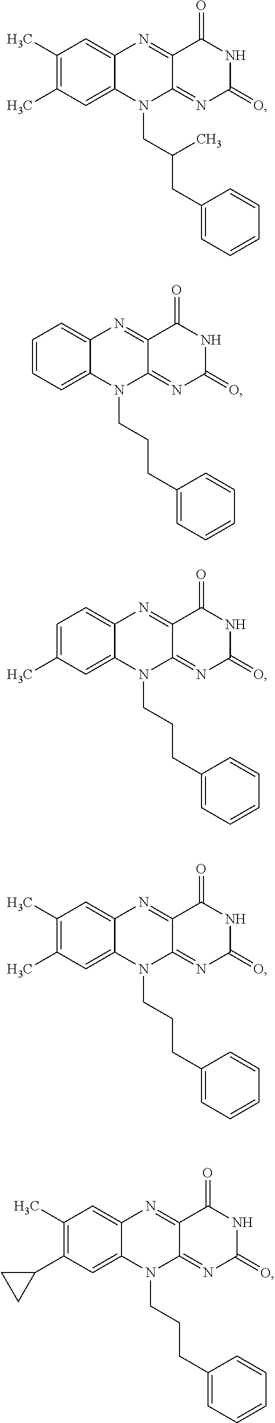 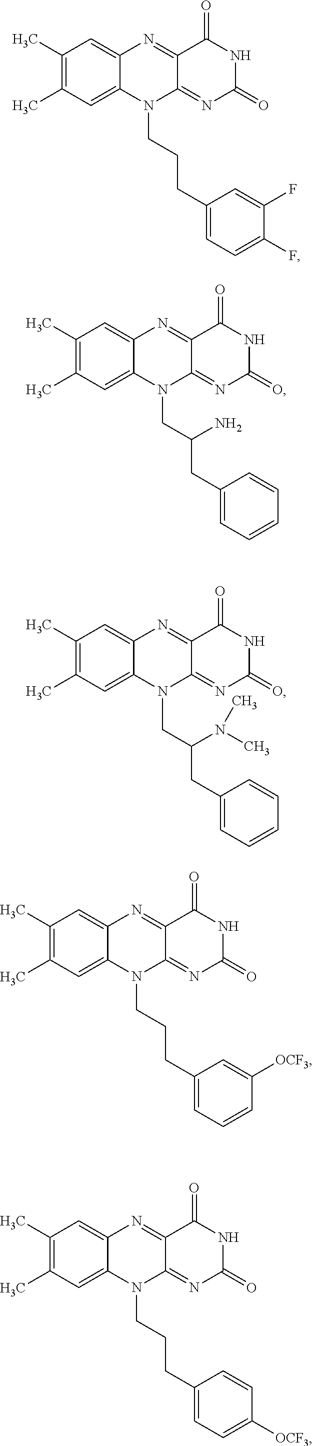 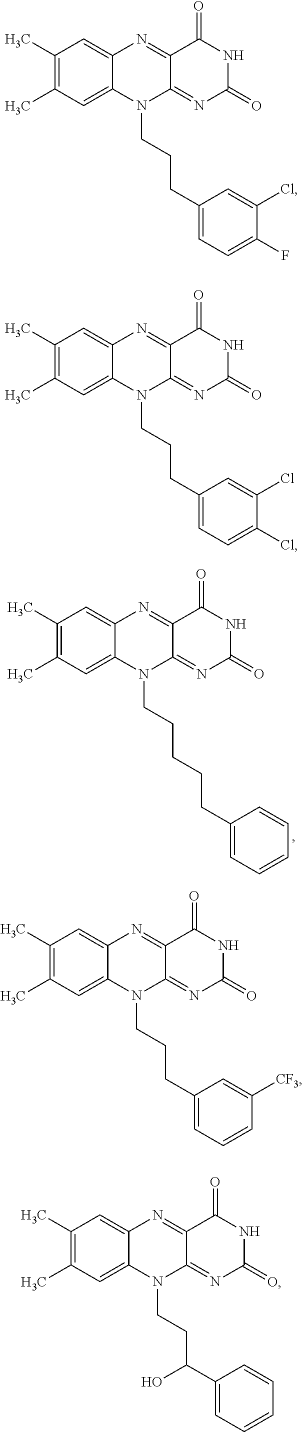 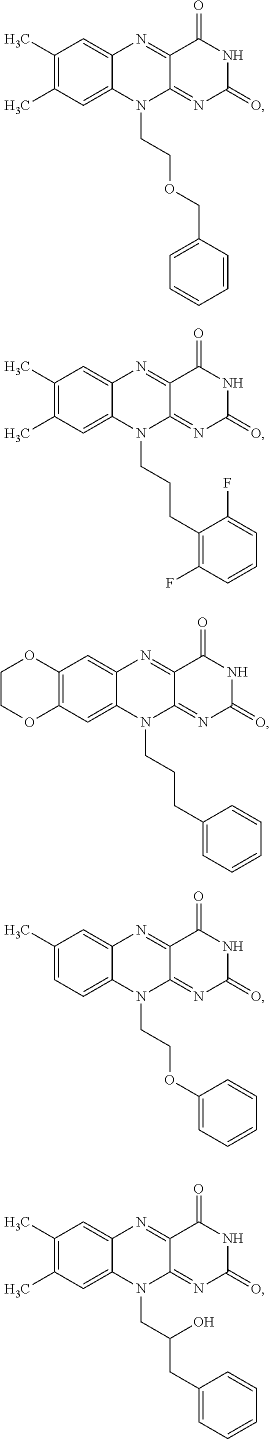 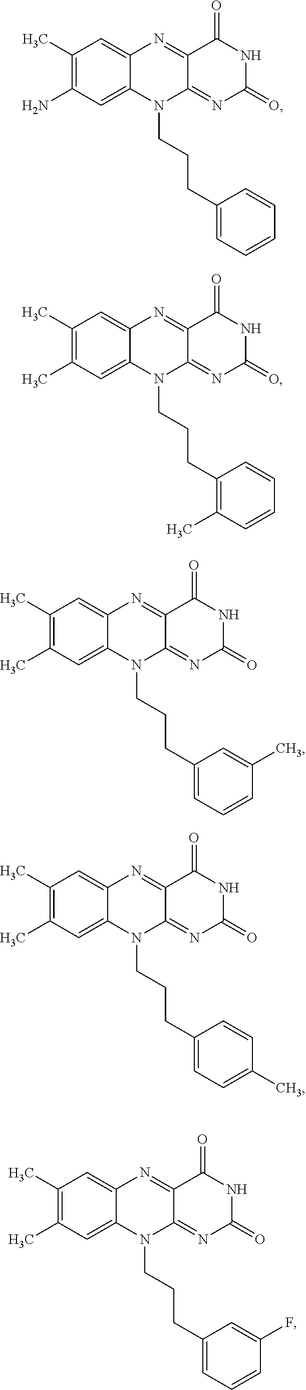 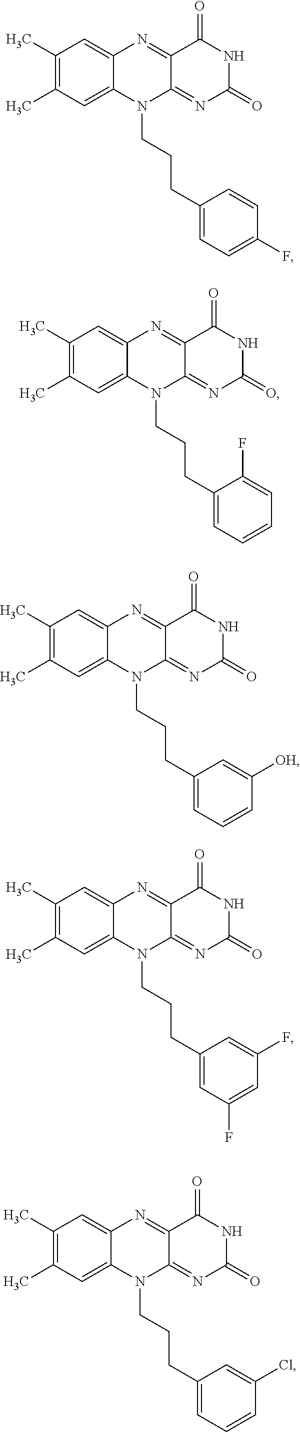 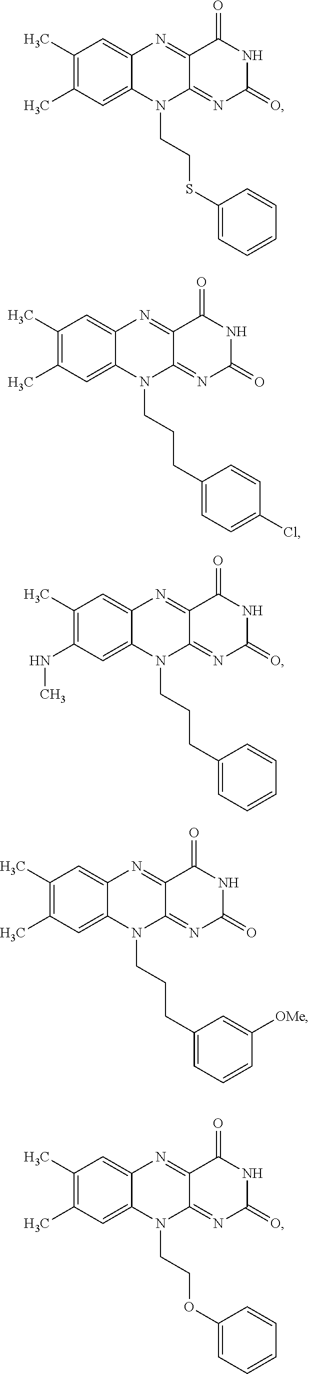 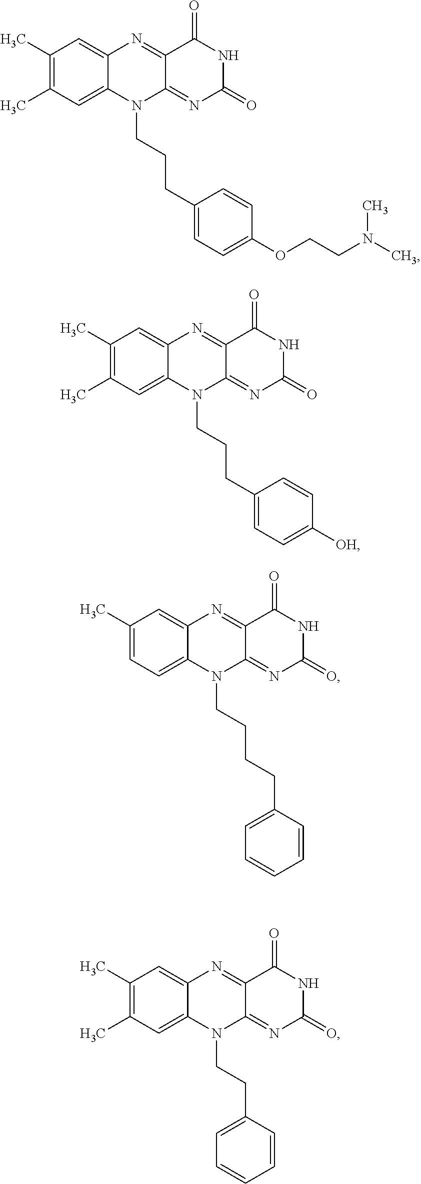 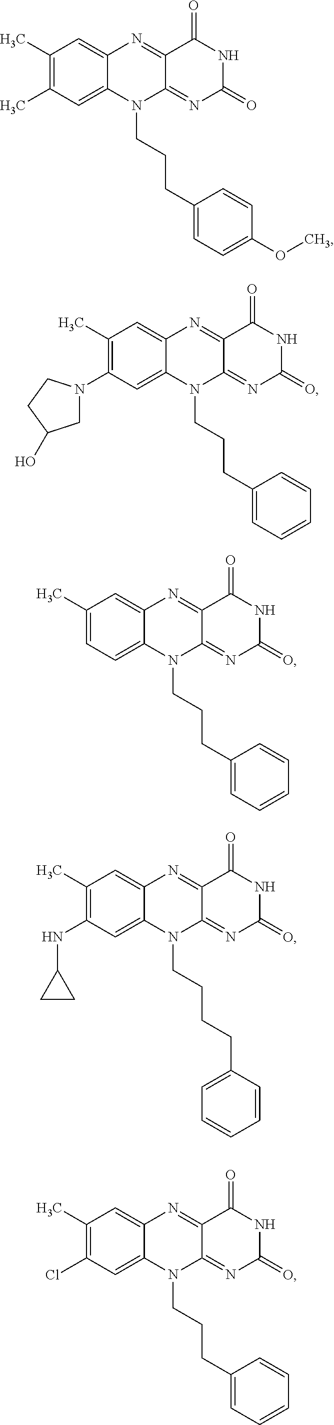 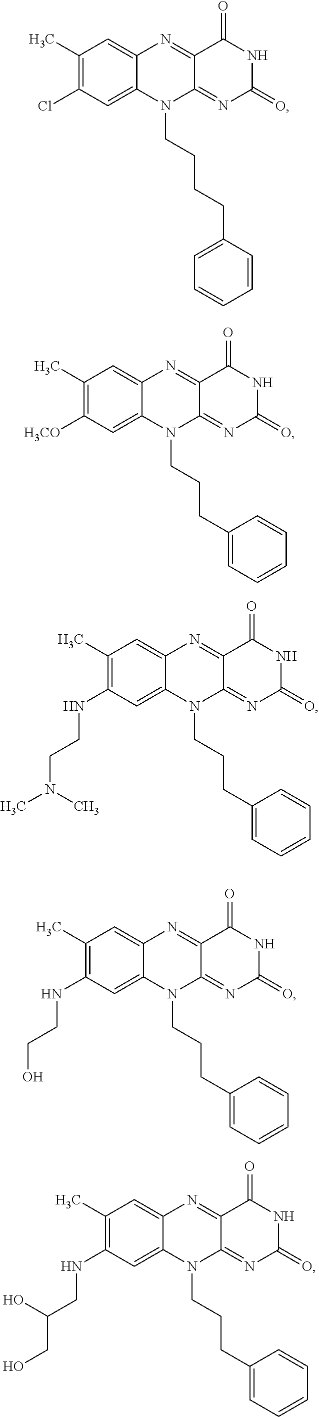 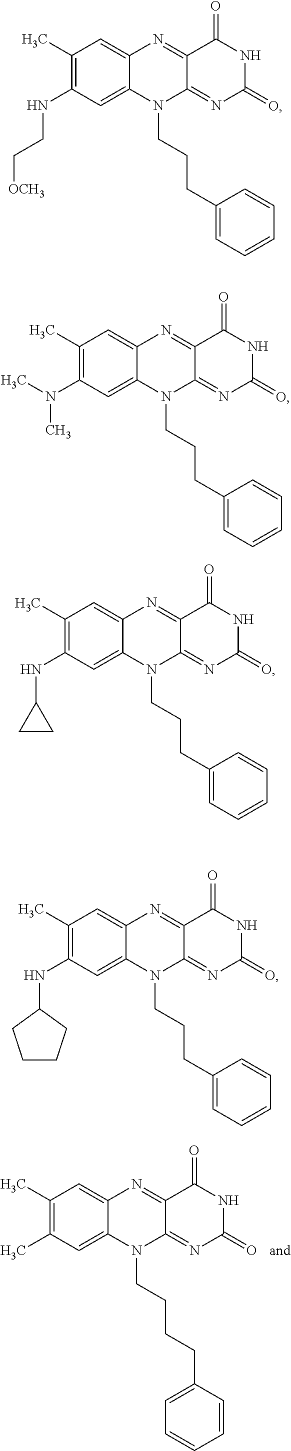 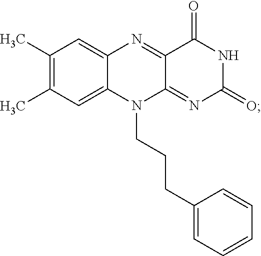 [SMILES: Cc1cc2nc3c(=O)[nH]c(=O)nc-3n(CCCc3ccccc3)c2cc1C]
in free or salt form.

8. The compound according to any one of claims 1-7 selected from any of following: 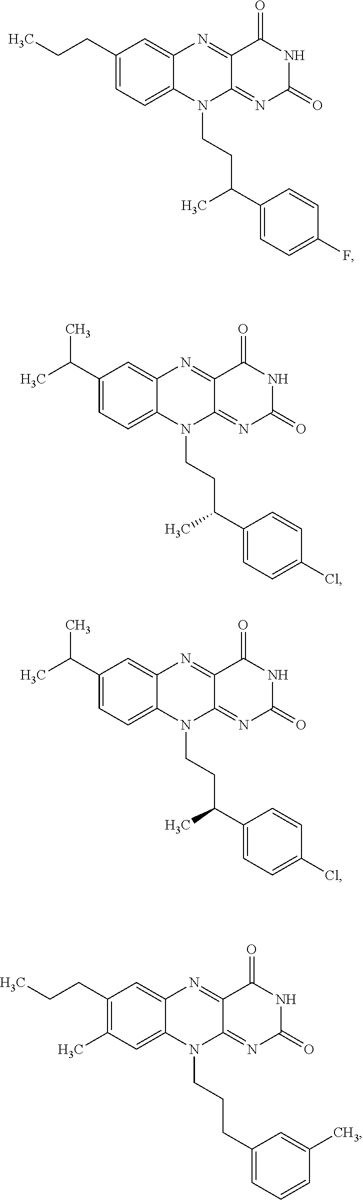 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCCc1cc2nc3c(=O)[nH]c(=O)nc-3n(CCCc3cccc(C)c3)c2cc1C] 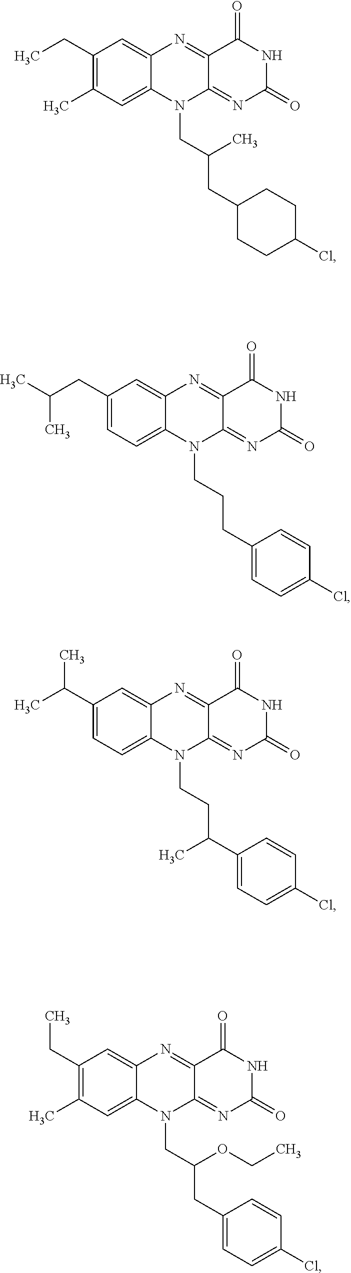 [SMILES: CCc1cc2nc3c(=O)[nH]c(=O)nc-3n(CC(C)CC3CCC(Cl)CC3)c2cc1C ; CC(C)Cc1ccc2c(c1)nc1c(=O)[nH]c(=O)nc-1n2CCCc1ccc(Cl)cc1 ; CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21] 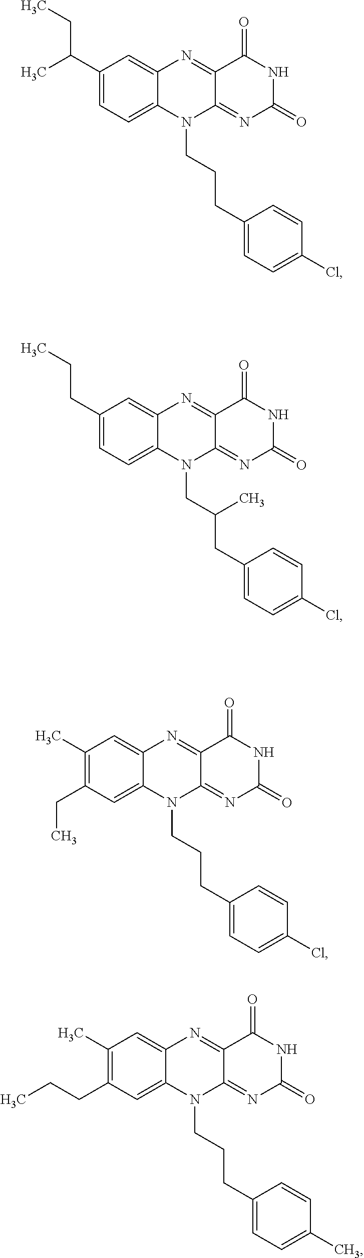 [SMILES: CCC(C)c1ccc2c(c1)nc1c(=O)[nH]c(=O)nc-1n2CCCc1ccc(Cl)cc1 ; CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1 ; CCCc1cc2c(cc1C)nc1c(=O)[nH]c(=O)nc-1n2CCCc1ccc(C)cc1] 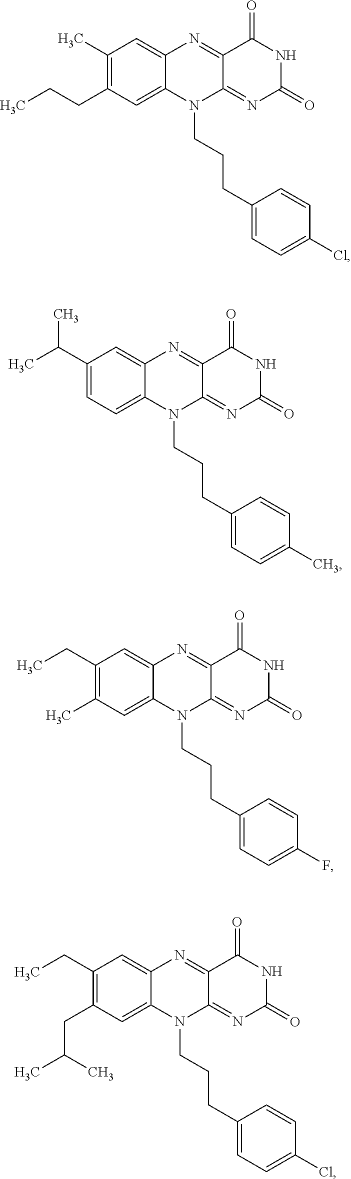 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; CCc1cc2nc3c(=O)[nH]c(=O)nc-3n(CCCc3ccc(Cl)cc3)c2cc1CC(C)C] 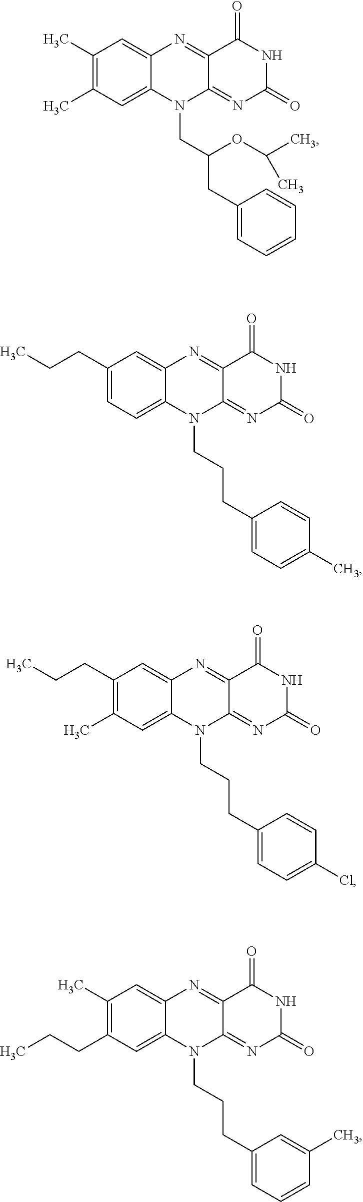 [SMILES: Cc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCCc1ccc(C)cc1 ; CCCc1cc2nc3c(=O)[nH]c(=O)nc-3n(CCCc3ccc(Cl)cc3)c2cc1C ; CCCc1cc2c(cc1C)nc1c(=O)[nH]c(=O)nc-1n2CCCc1cccc(C)c1] 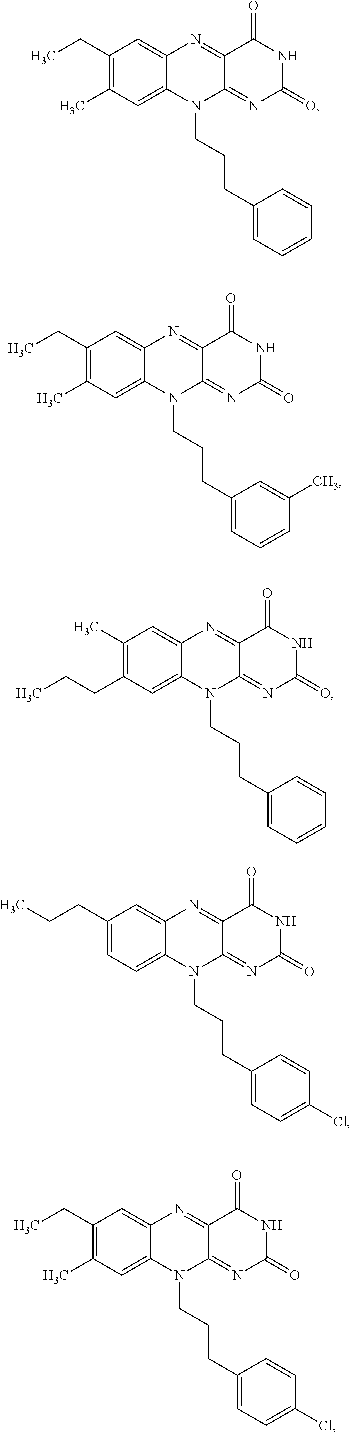 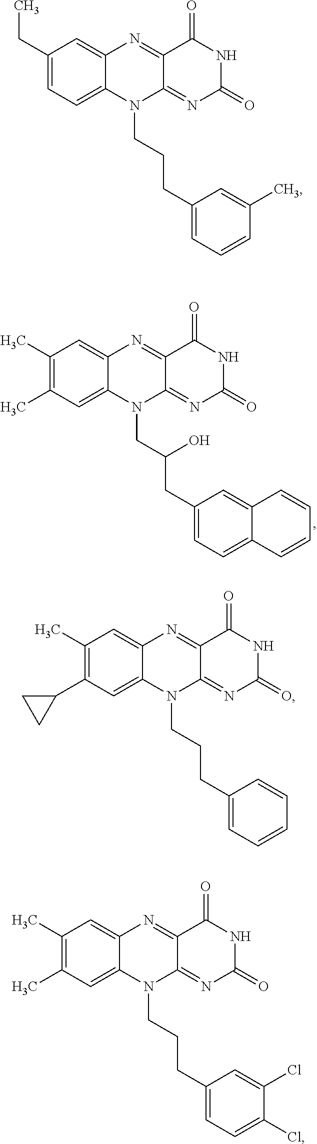 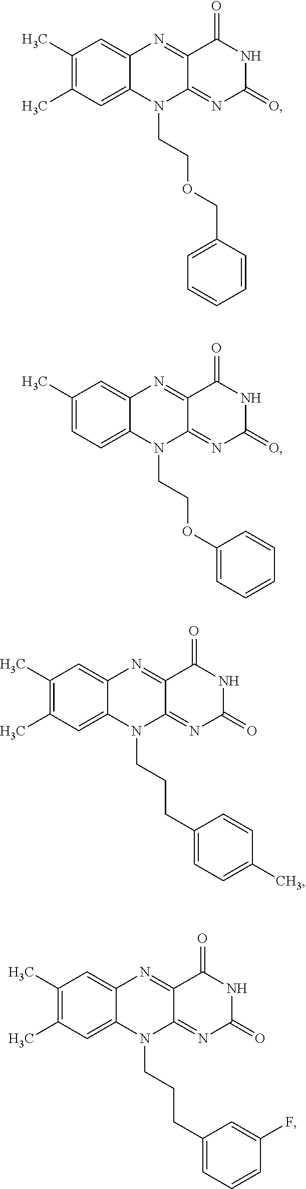 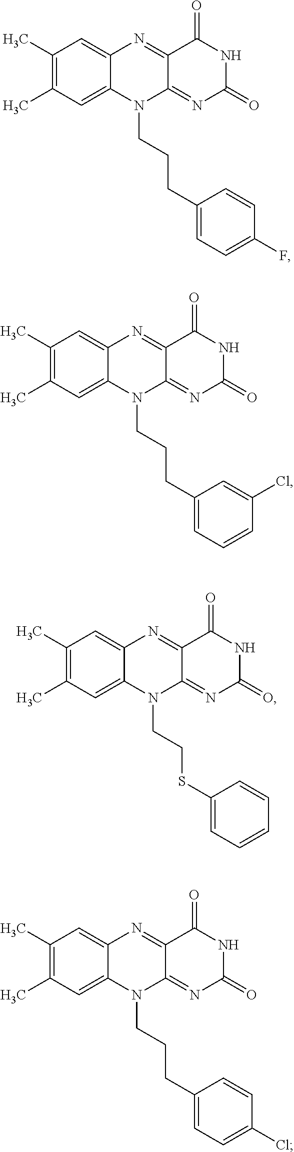 [SMILES: Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(Cl)c3)c2cc1C ; Cc1cc2nc3c(=O)[nH]c(=O)nc-3n(CCSc3ccccc3)c2cc1C ; Cc1cc2nc3c(=O)[nH]c(=O)nc-3n(CCCc3ccc(Cl)cc3)c2cc1C] 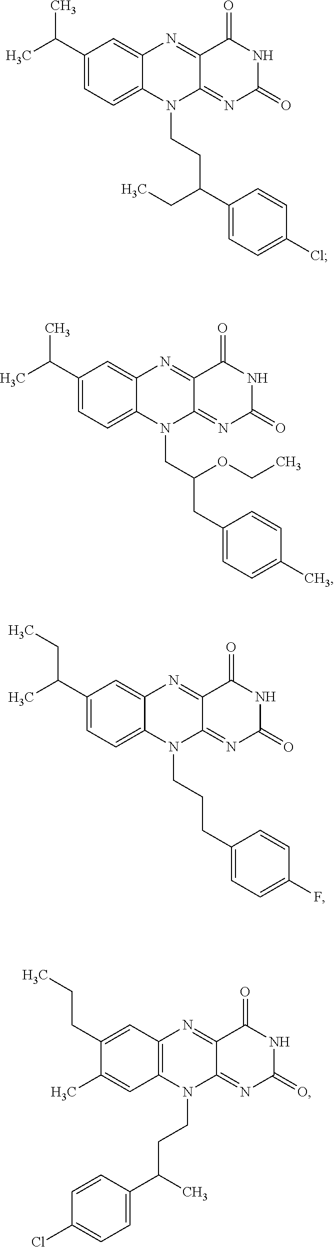 [SMILES: CCC(CCn1c2nc(=O)[nH]c(=O)c-2nc2cc(C(C)C)ccc21)c1ccc(Cl)cc1 ; CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C] 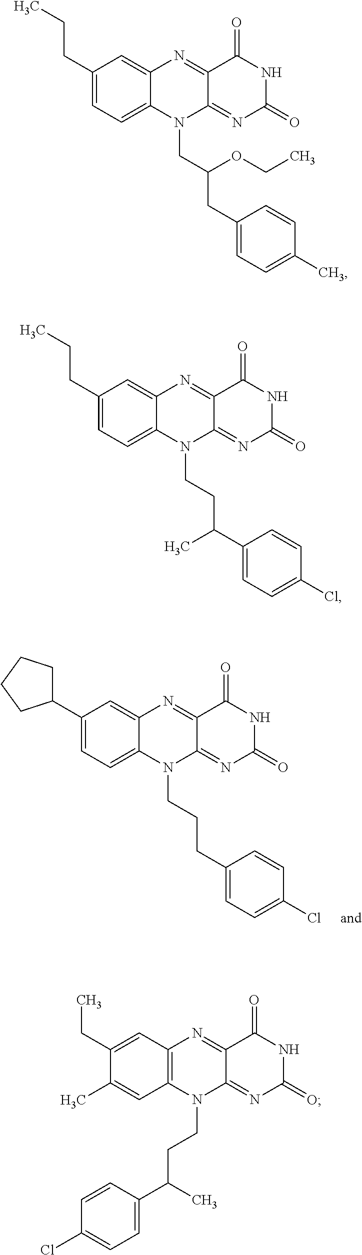 [SMILES: CCCc1ccc2c(c1)nc1c(=O)[nH]c(=O)nc-1n2CC(Cc1ccc(C)cc1)OCC ; CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C]
in free or salt form.

9. The compound according to any one of claims 1-8 selected from any of following: in free or salt form. 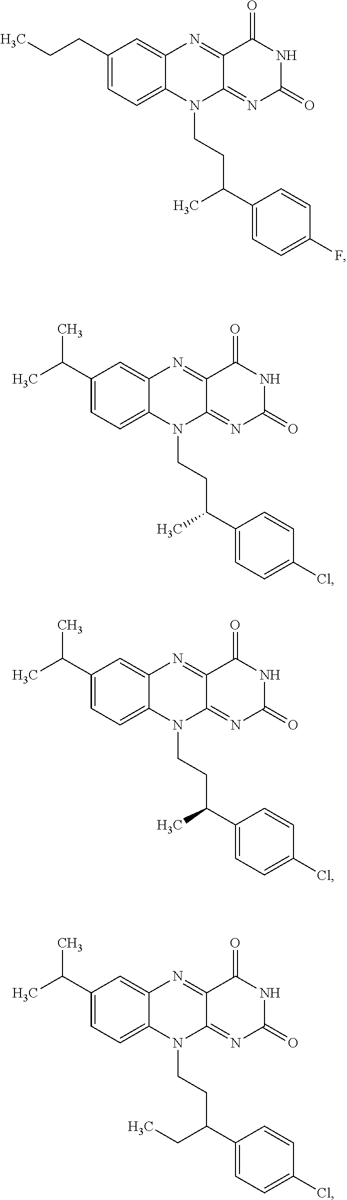 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1] 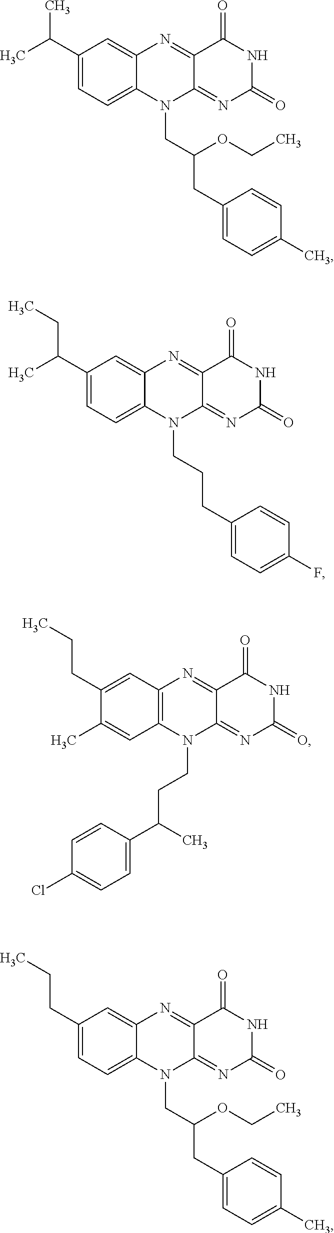 [SMILES: CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C ; CCCc1ccc2c(c1)nc1c(=O)[nH]c(=O)nc-1n2CC(Cc1ccc(C)cc1)OCC] 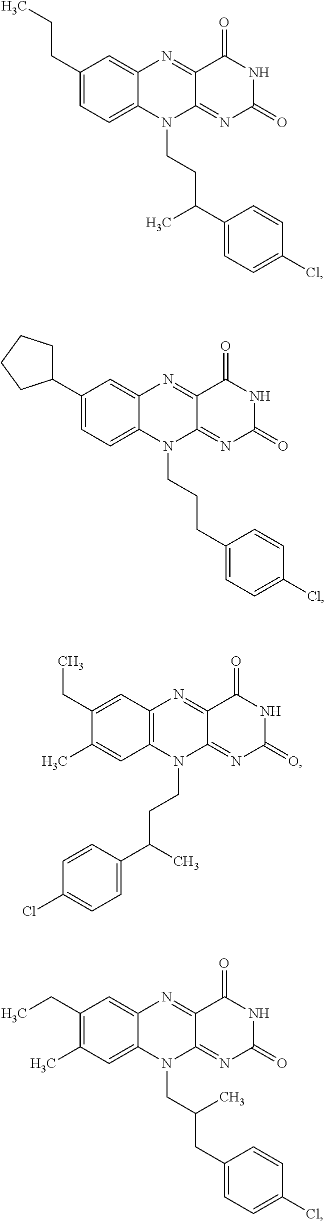 [SMILES: CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C ; CCc1cc2nc3c(=O)[nH]c(=O)nc-3n(CC(C)Cc3ccc(Cl)cc3)c2cc1C] 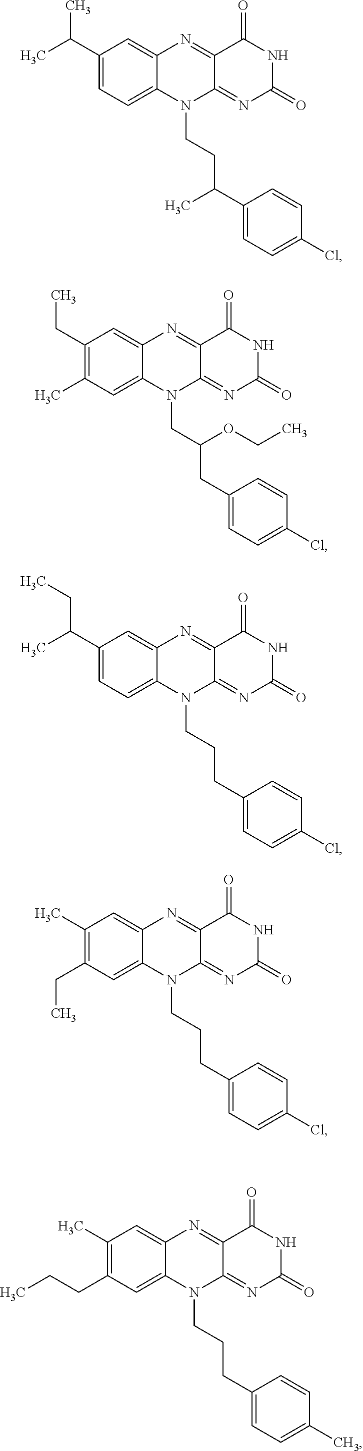 [SMILES: CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1 ; CCc1cc2c(cc1C)nc1c(=O)[nH]c(=O)nc-1n2CCCc1ccc(Cl)cc1 ; CCCc1cc2c(cc1C)nc1c(=O)[nH]c(=O)nc-1n2CCCc1ccc(C)cc1] 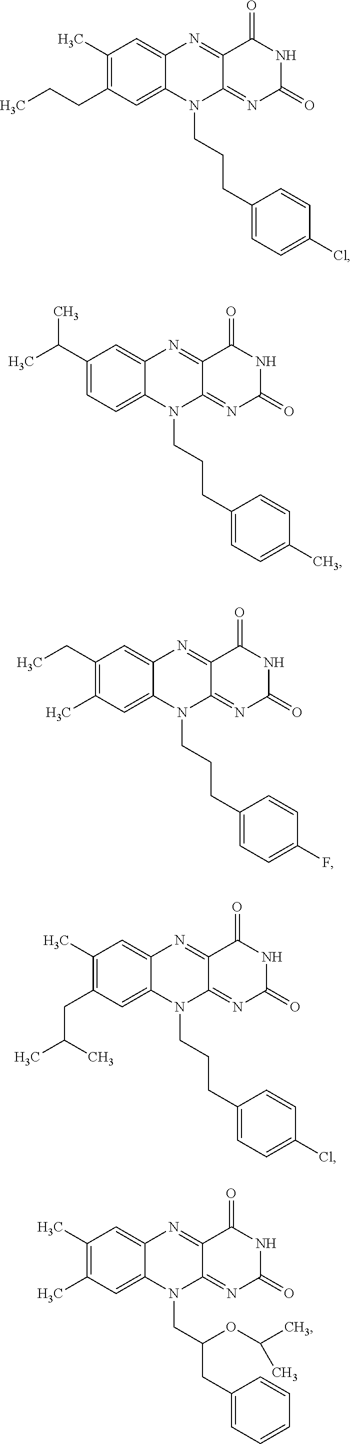 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C ; Cc1cc2nc3c(=O)[nH]c(=O)nc-3n(CC(Cc3ccccc3)OC(C)C)c2cc1C] 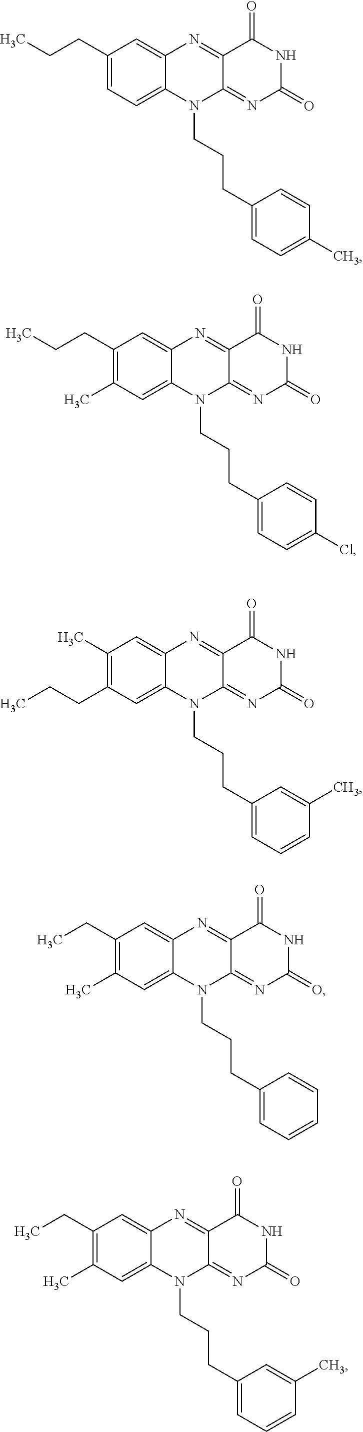 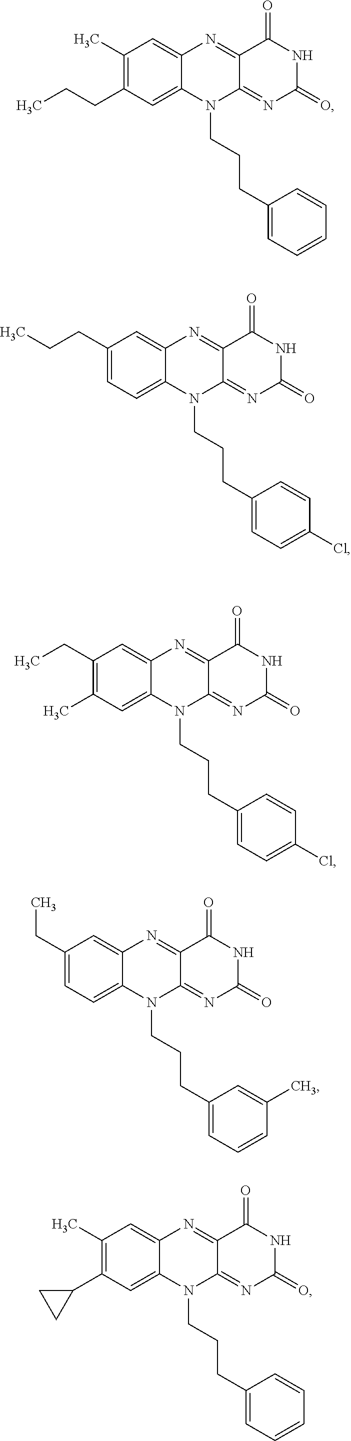 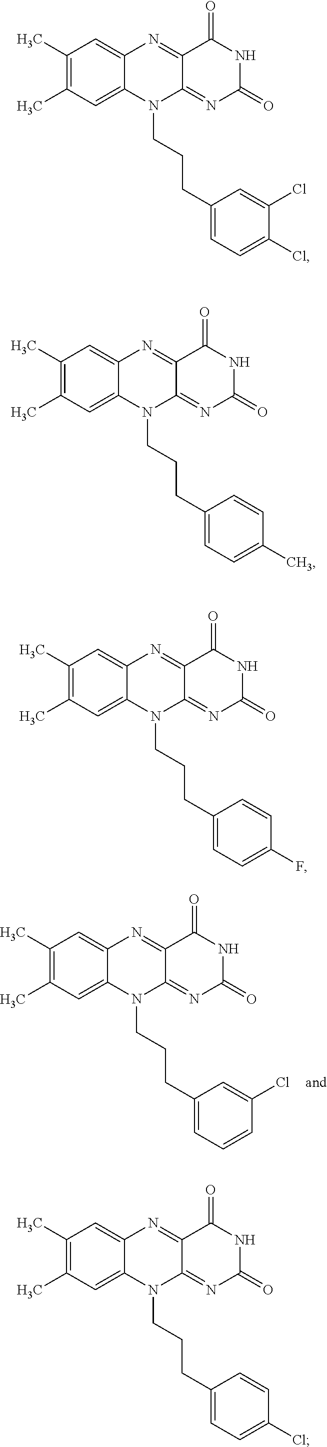
in free or salt form.

10. A pharmaceutical composition comprising a compound of Formula P: 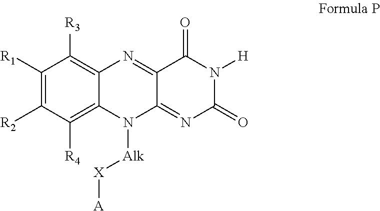
wherein:
(i) Alk is C₁₋₄alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl, ethyl or isobutyl), (e.g., benzyl) and/or —N(R_{c})(R_{d}); or
Alk is C₁₋₄alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄alkoxy (e.g., methoxy, ethoxy, propoxy, isobutoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
C₁₋₄alkyl (e.g., methyl, ethyl, t-butyl or n-prop-2-en-1-yl),
C₁₋₄alkoxy (e.g., methoxy),
hydroxy,
—O—C₁₋₄alkyl-N(R_{c})(R_{d}), for example —OCH₂CH₂N(CH₃)₂,
halo (e.g., Cl, F),
haloC₁₋₄alkyl (e.g., CF₃),
—O-haloC₁₋₄alkyl (e.g., —OCF₃),
cyano,
—O—(CH₂CH₂O)₁₋₃—C₁₋₄alkyl (e.g., —OCH₂CH₂OCH₃ or —O(CH₂CH₂O)₃CH₃), and/or
—CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
H,
C₁₋₆alkyl, e.g., C₁₋₄alkyl (for example, methyl, ethyl, n-propyl, isopropyl, isobutyl, t-butyl, 1-methylpropyl or n-hexyl),
C₃₋₈cycloalkyl (e.g., cyclopropyl or cyclopentyl),
aryl (e.g., phenyl), or
C₁₋₄alkoxy (e.g., methoxy);
(v) R₂ is:
H,
C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
—C₀₋₄alkyl-C₃₋₈cycloalkyl (e.g., cyclopropyl),
—C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
—C₀₋₄alkyl-N(Rₐ)(R_{b}), for example —C₀alkyl-N(Rₐ)(R_{b}) or —C₁alkyl-N(Rₐ)(R_{b}),
C₁₋₄alkoxy (e.g., methoxy),
halo (e.g., Cl),
—O—(CH₂CH₂O)₁₋₃—C₁₋₄alkyl (e.g., —OCH₂CH₂OCH₃ or —O(CH₂CH₂O)₃CH₃),
—N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
—N(Rₑ)—C(O)—O—C₁₋₄alkyl (e.g., —N(H)—C(O)—O—C(H)(CH₃)CH₃),
—N(Rₑ)—C(O)-aryl wherein said aryl is optionally substituted with one or more halo (e.g., F), for example —N(H)—C(O)-(4-fluorophenyl),
—C₁₋₆alkyl-OC₁₋₄alkyl (e.g., —CH₂CH₂CH₂CH₂—O—CH₃),
—O—CH₂CH₂—O—CH₂-phenyl,
—O-haloC₁₋₄alkyl (e.g., —OCH₂CF₃),
—CH₂—O—C(O)—C₁₋₄alkyl (e.g., —CH₂—O—C(O)—CH₃),
—C(O)O—C₁₋₄alkyl (e.g., —C(O)OCH₃), or
C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
H,
C₁₋₄alkyl (e.g., methyl) optionally substituted with one or more hydroxy groups for example, 2,3-dihydroxyprop-1-yl,
C₃₋₈cycloalkyl (e.g., cyclopropyl or cyclopentyl),
C₁₋₄alkoxy-C₁₋₄alkyl (e.g., methoxyethyl),
hydroxy-C₁₋₄alkyl (e.g., hydroxyethyl),
N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
in free or pharmaceutically acceptable salt form, in admixture with a pharmaceutically acceptable diluent or carrier.

11. The pharmaceutical composition according to claim 10, wherein said compound is a compound of Formula Q: 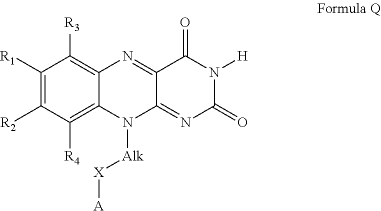
wherein:
(i) Alk is C₁₋₆alkylene (e.g., C₂₋₅alkylene, for example ethylene i.e., —CH₂CH₂—, n-propylene, i.e., —CH₂CH₂CH₂—, n-butylene, e.g., —CH₂CH₂CH₂CH₂— or n-pentylene, i.e., —CH₂CH₂CH₂CH₂CH₂—) optionally substituted with one or more C₁₋₄alkyl (e.g., methyl or isobutyl) and/or —N(R_{c})(R_{d}); or
Alk is C₁₋₆alkylene (e.g., C₂₋₅alkylene, for example n-propylene, i.e., —CH₂CH₂CH₂—, n-butylene, i.e., —CH₂CH₂CH₂CH₂— or n-pentylene, i.e., —CH₂CH₂CH₂CH₂CH₂—) optionally substituted with one hydroxy or C₁₋₄alkoxy (e.g., methoxy, ethoxy or isopropyloxy) group; and
(ii) X is a single bond, —S—, —S(O)₂—, —S(O)— or —O—;
(iii) A is aryl (e.g., phenyl or naphthyl) or aryl-C₁₋₄alkyl (e.g., benzyl or naphthylmethyl), wherein the aryl group of said aryl or arylalkyl is optionally substituted with one or more
C₁₋₄alkyl (e.g., methyl, t-butyl or n-prop-2-en-1-yl),
C₁₋₄alkoxy (e.g., methoxy),
hydroxy,
—O—C₁₋₄alkyl-N(R_{c})(R_{d}), for example —OCH₂CH₂N(CH₃)₂,
halo (e.g., Cl, F),
haloC₁₋₄alkyl (e.g., CF₃),
—O-haloC₁₋₄alkyl (e.g., —OCF₃),
cyano,
—O—(CH₂CH₂O)₁₋₃—C₁₋₄alkyl (e.g., —OCH₂CH₂OCH₃ or —O(CH₂CH₂O)₃CH₃), and/or
—CH₂-heteroC₃₋₈cycloalkyl wherein said cycloalkyl is optionally substituted with one or more C₁₋₄alkyl (e.g., methyl), for example, [2,6-dimethylmorpholin-4-yl]methyl, e.g. [(2R,6S)-2,6-dimethylmorpholin-4-yl]methyl) or [(2R,6R)-2,6-dimethylmorpholin-4-yl]methyl);
(iv) R₁ is:
H,
C₁₋₆alkyl, e.g., C₁₋₄alkyl (for example, methyl, ethyl, n-propyl, isopropyl, isobutyl, t-butyl, 1-methylpropyl or n-hexyl),
C₃₋₈cycloalkyl (e.g., cyclopropyl),
aryl (e.g., phenyl), or
C₁₋₄alkoxy (e.g., methoxy);
(v) R₂ is:
H,
C₁₋₆alkyl, e.g., C₁₋₄alkyl (for example, methyl, ethyl, n-propyl, isopropyl, n-prop-2-en-1-yl, n-butyl, isobutyl, n-but-2-en-1-yl, n-hexyl),
—C₀₋₄alkyl-C₃₋₈cycloalkyl (e.g., cyclopropyl),
—C₁₋₄alkyl-heteroC₃₋₈cycloalkyl, wherein said heterocycloalkyl is optionally substituted with one or more hydroxy and/or C₁₋₄alkyl (e.g., methyl) groups, for example, [2,6-dimethylmorpholin-4-yl]methyl,
—C₀₋₄alkyl-N(Rₐ)(R_{b}), for example —C₀alkyl-N(Rₐ)(R_{b}) or —C₁alkyl-N(Rₐ)(R_{b}),
C₁₋₄alkoxy (e.g., methoxy),
halo (e.g., Cl),
—O—(CH₂CH₂O)₁₋₃—C₁₋₄alkyl (e.g., —OCH₂CH₂OCH₃ or —O(CH₂CH₂O)₃CH₃),
—N(Rₑ)—C(O)—C₁₋₄alkyl (e.g., —N(H)—C(O)—CH₃, —N(H)—C(O)—CH₂CH₃ or —N(H)—C(O)—C(H)(CH₃)CH₃),
—N(Rₑ)—C(O)—O—C₁₋₄alkyl (e.g., —N(H)—C(O)—O—C(H)(CH₃)CH₃),
—N(Rₑ)—C(O)-aryl wherein said aryl is optionally substituted with one or more halo (e.g., F), for example —N(H)—C(O)-(4-fluorophenyl),
—C₁₋₆alkyl-OC₁₋₄alkyl (e.g., —CH₂CH₂CH₂CH₂—O—CH₃),
—O—CH₂CH₂—O—CH₂-phenyl,
—O-haloC₁₋₄alkyl (e.g., —OCH₂CF₃),
—CH₂—O—C(O)—C₁₋₄alkyl (e.g., —CH₂—O—C(O)—CH₃),
—C(O)O—C₁₋₄alkyl (e.g., —C(O)OCH₃), or
C₃₋₈heterocycloalkyl (e.g., pyrrolidinyl, for example pyrrolidin-1-yl) wherein said heterocycloalkyl is optionally substituted with one or more hydroxy, for example 3-hydroxypyrrolidin-1-yl; or
(vi) Optionally, R₁ and R₂ are linked together so that together with the carbon atoms to which they are attached they form a cyclic structure (e.g., R₁ and R₂ are linked together to form ethylenedioxy);
(vii) Optionally, R₂ and A may be linked together so that together with the carbon atoms to which they are attached they form a cyclic structure (e.g., R₂ and A are linked together to form, e.g., 14-methyl-1,17,20,22-tetraazapentacyclo[11.10.2.25,8.016,24.018,23]heptacosa-5,7,10,13(25),14,16(24),17,22,26-nonaene-19,21-dione or 14-methyl-1,17,20,22-tetraazapentacyclo[11.10.2.25,8.016,24.018,23]heptacosa-5,7,13(25),14,16(24),17,22,26-octaene-19,21-dione;
(viii) Rₐ and R_{b} are independently:
H,
C₁₋₄alkyl (e.g., methyl) optionally substituted with one or more hydroxy groups for example, 2,3-dihydroxyprop-1-yl,
C₃₋₈cycloalkyl (e.g., cyclopropyl or cyclopentyl),
C₁₋₄alkoxy-C₁₋₄alkyl (e.g., methoxyethyl),
hydroxy-C₁₋₄alkyl (e.g., hydroxyethyl),
N(R_{c})(R_{d})—C₁₋₄alkyl (e.g., dimethylaminoethyl);
(ix) R_{c} and R_{d} are independently H, C₁₋₄alkyl (e.g., methyl) or arylC₁₋₄alkyl (e.g., benzyl);
(x) R₃ and R₄ are independently H or C₁₋₄alkyl (e.g., methyl);
(xi) Rₑ is H or C₁₋₄alkyl,
in free or pharmaceutically acceptable salt form.

12. The pharmaceutical composition according to claim 10 or 11, wherein said compound is a compound of Formula I: 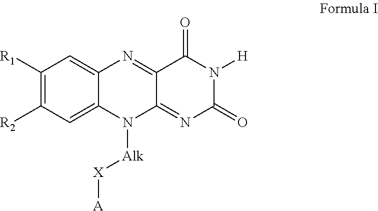
wherein:
(i) Alk is C₁₋₆alkylene (e.g., ethylene, n-propylene, n-butylene, n-pentylene) optionally substituted with one or more C₁₋₄alkyl, —N(R_{c})(R_{d}); or
Alk is C₁₋₆alkylene (e.g., n-propylene, n-butylene, n-pentylene) optionally substituted with one hydroxy or C₁₋₄alkoxy group;
(ii) X is a single bond, —S— or —O—;
(iii) A is aryl (e.g., phenyl) or aryl-C₁₋₄alkyl (e.g., benzyl), wherein the aryl group of said aryl or arylalkyl is optionally substituted with one or more C₁₋₄alkyl (e.g., methyl), C₁₋₄alkoxy (e.g., methoxy), hydroxy, —O—C₁₋₄alkyl-N(R_{c})(R_{d}), halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl) or C₁₋₄alkoxy (e.g., methoxy);
(v) R₂ is H, C₁₋₄alkyl (e.g., methyl), —C₀₋₄alkyl-C₃₋₈cycloalkyl (e.g., cyclopropyl), —C₀₋₄alkyl-N(Rₐ)(R_{b}), C₁₋₄alkoxy (e.g., methoxy), halo (e.g., Cl), C₃₋₈heterocycloalkyl (e.g., pyrrolidinyl, for example pyrrolidin-1-yl) wherein said heterocycloalkyl is optionally substituted with one or more hydroxy; or
(vi) Optionally, R₁ and R₂ are linked together so that together with the carbon atoms to which they are attached they form a cyclic structure (e.g., R₁ and R₂ are linked together to form ethylenedioxy);
(vii) Rₐ and R_{b} are independently H, C₁₋₄alkyl (e.g., methyl), C₃₋₈cycloalkyl (e.g., cyclopropyl, cyclopentyl), C₁₋₄alkoxy-C₁₋₄alkyl (e.g., methoxyethyl), hydroxy-C₁₋₄alkyl (e.g., hydroxyethyl), N(R_{c})(R_{d})—C₁₋₄alkyl (e.g., dimethylaminoethyl);
(viii) R_{c} and R_{d} are independently H or C₁₋₄alkyl (e.g., methyl);
in free or pharmaceutically acceptable salt form.

13. The pharmaceutical composition according to any one of claims 10-12, wherein:
Alk is C₂₋₃alkylene (e.g., ethylene, i.e., CH₂CH₂—, n-propylene, i.e., —CH₂CH₂CH₂—) optionally substituted with one or more C₁₋₄alkyl (e.g., methyl, ethyl or isobutyl); or
Alk is C₂₋₃alkylene (e.g., ethylene, i.e., CH₂CH₂— or n-propylene, i.e., —CH₂CH₂CH₂—) optionally substituted with one C₁₋₄alkoxy (e.g., ethoxy or isopropyloxy) group;
X is a single bond, —S— or —O—;
A is aryl (e.g., phenyl), wherein the aryl group is optionally substituted with one or more
C₁₋₄alkyl (e.g., methyl),
halo (e.g., Cl, F),
R₁ is:
C₁₋₆alkyl, e.g., C₁₋₄alkyl (for example, methyl, ethyl, n-propyl, isopropyl, isobutyl, t-butyl, 1-methylpropyl),
C₃₋₈cycloalkyl (e.g., cyclopentyl),
R₂ is:
H,
C₁₋₆alkyl, e.g., C₁₋₄alkyl (for example, methyl, ethyl, n-propyl or isopropyl),
—C₀₋₄alkyl-C₃₋₈cycloalkyl (e.g., cyclopropyl),
in free or pharmaceutically acceptable salt form.

14. The pharmaceutical composition according to any one of claims 10-13, wherein:
Alk is C₃alkylene (e.g., n-propylene, i.e., —CH₂CH₂CH₂—) optionally substituted with one or more C₁₋₄alkyl (e.g., methyl or ethyl); or
Alk is C₃alkylene (e.g., n-propylene, i.e., —CH₂CH₂CH₂—) optionally substituted with one C₁₋₄alkoxy (e.g., ethoxy or isopropyloxy) group;
X is a single bond;
A is aryl (e.g., phenyl), wherein the aryl group is optionally substituted with one or more
C₁₋₄alkyl (e.g., methyl),
halo (e.g., Cl, F),
R₁ is:
C₁₋₆alkyl, e.g., C₁₋₄alkyl (for example, methyl, ethyl, n-propyl, isopropyl or 1-methylpropyl),
C₃₋₈cycloalkyl (e.g., cyclopentyl),
R₂ is:
H,
C₁₋₆alkyl, e.g., C₁₋₄alkyl (for example, methyl, ethyl, n-propyl or isopropyl),
—C₀₋₄alkyl-C₃₋₈cycloalkyl (e.g., cyclopropyl),
in free or pharmaceutically acceptable salt form.

15. The pharmaceutical composition according to any one of claims 10-14, wherein:
Alk is —CH₂CH₂CH₂—;
X is a single bond;
A is aryl (e.g., phenyl);
R₁ is C₁₋₆alkyl, e.g., C₁₋₄alkyl (for example, methyl),
R₂ is C₁₋₆alkyl, e.g., C₁₋₄alkyl (for example, methyl),
R₃ and R₄ are H
in free or pharmaceutically acceptable salt form.

16. The pharmaceutical composition according to any one of claims 10-15, wherein said compound is selected from any of the following: 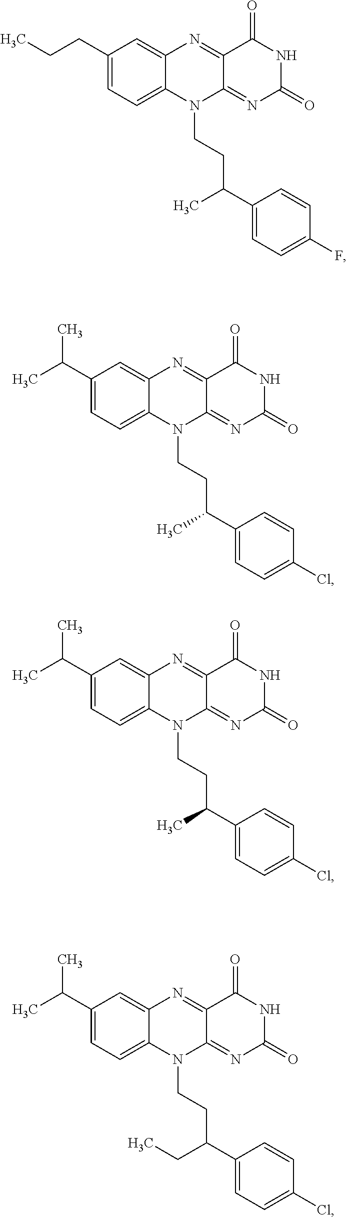 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1] 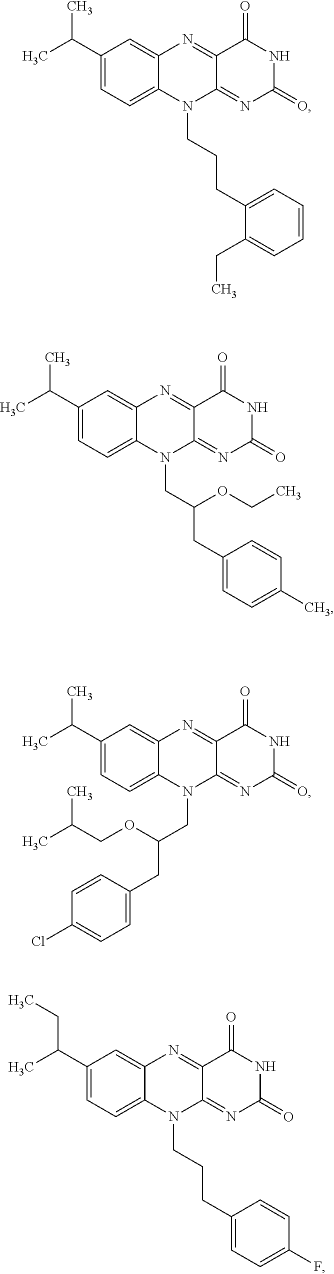 [SMILES: CCc1ccccc1CCCn1c2nc(=O)[nH]c(=O)c-2nc2cc(C(C)C)ccc21 ; CCOC(Cc1ccc(C)cc1)Cn1c2nc(=O)[nH]c(=O)c-2nc2cc(C(C)C)ccc21 ; CC(C)COC(Cc1ccc(Cl)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1] 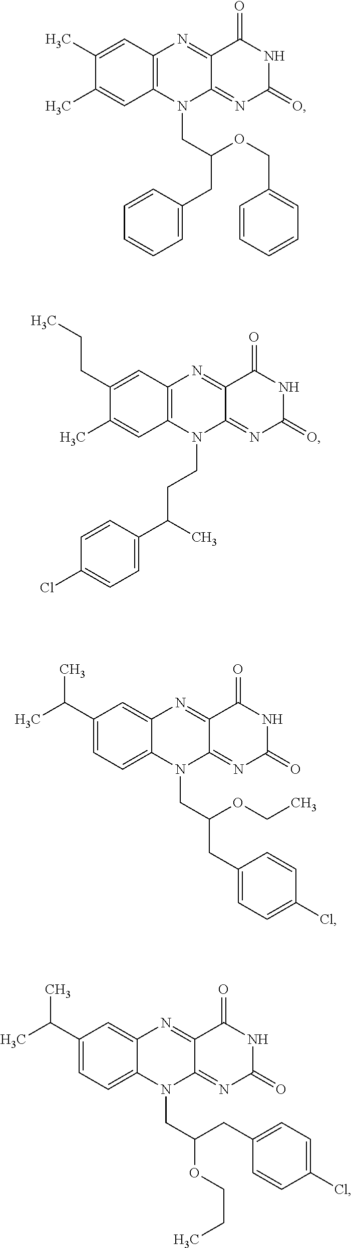 [SMILES: Cc1cc2nc3c(=O)[nH]c(=O)nc-3n(CC(Cc3ccccc3)OCc3ccccc3)c2cc1C ; CCCc1cc2nc3c(=O)[nH]c(=O)nc-3n(CCC(C)c3ccc(Cl)cc3)c2cc1C ; CCOC(Cc1ccc(Cl)cc1)Cn1c2nc(=O)[nH]c(=O)c-2nc2cc(C(C)C)ccc21 ; CCCOC(Cc1ccc(Cl)cc1)Cn1c2nc(=O)[nH]c(=O)c-2nc2cc(C(C)C)ccc21] 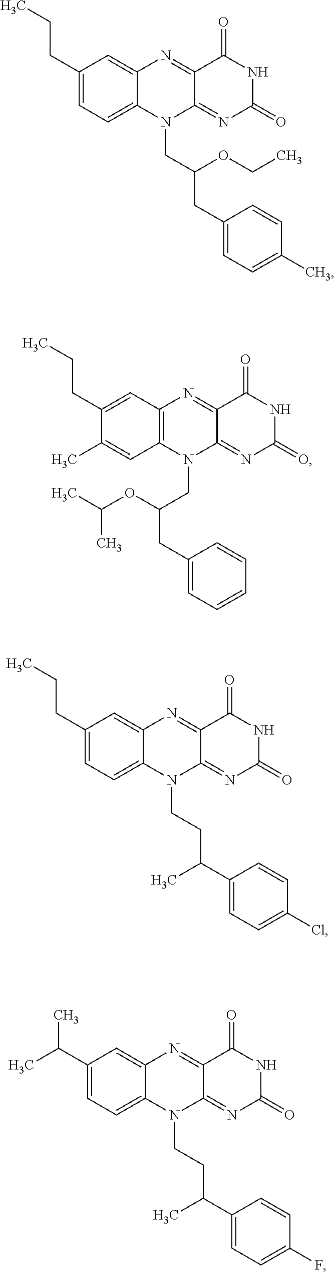 [SMILES: CCCc1ccc2c(c1)nc1c(=O)[nH]c(=O)nc-1n2CC(Cc1ccc(C)cc1)OCC ; CCCc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCC(C)c1ccc(Cl)cc1 ; CC(C)c1ccc2c(c1)nc1c(=O)[nH]c(=O)nc-1n2CCC(C)c1ccc(F)cc1] 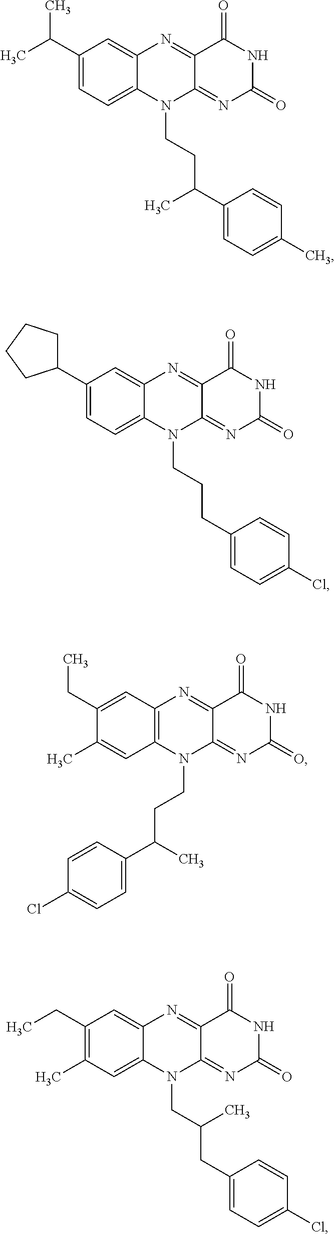 [SMILES: Cc1ccc(C(C)CCn2c3nc(=O)[nH]c(=O)c-3nc3cc(C(C)C)ccc32)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C ; CCc1cc2nc3c(=O)[nH]c(=O)nc-3n(CC(C)Cc3ccc(Cl)cc3)c2cc1C] 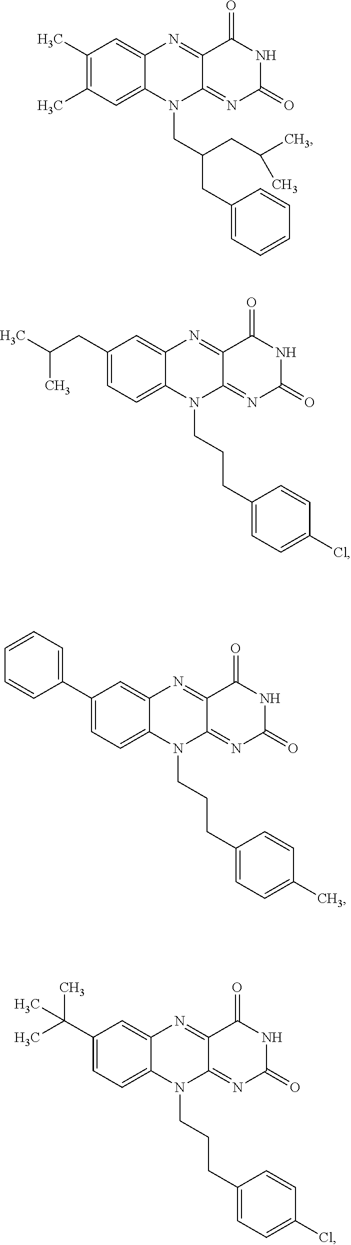 [SMILES: Cc1cc2nc3c(=O)[nH]c(=O)nc-3n(CC(Cc3ccccc3)CC(C)C)c2cc1C ; CC(C)Cc1ccc2c(c1)nc1c(=O)[nH]c(=O)nc-1n2CCCc1ccc(Cl)cc1 ; Cc1ccc(CCCn2c3nc(=O)[nH]c(=O)c-3nc3cc(-c4ccccc4)ccc32)cc1 ; CC(C)(C)c1ccc2c(c1)nc1c(=O)[nH]c(=O)nc-1n2CCCc1ccc(Cl)cc1] 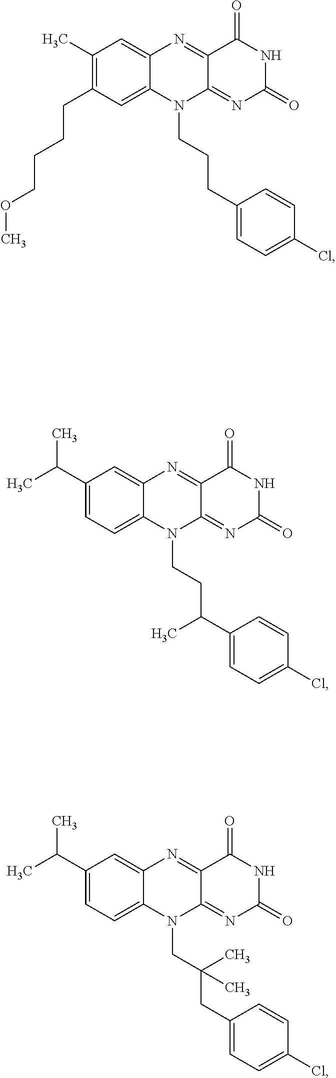 [SMILES: COCCCCc1cc2c(cc1C)nc1c(=O)[nH]c(=O)nc-1n2CCCc1ccc(Cl)cc1 ; CC(C)c1ccc2c(c1)nc1c(=O)[nH]c(=O)nc-1n2CCC(C)c1ccc(Cl)cc1 ; CC(C)c1ccc2c(c1)nc1c(=O)[nH]c(=O)nc-1n2CC(C)(C)Cc1ccc(Cl)cc1] 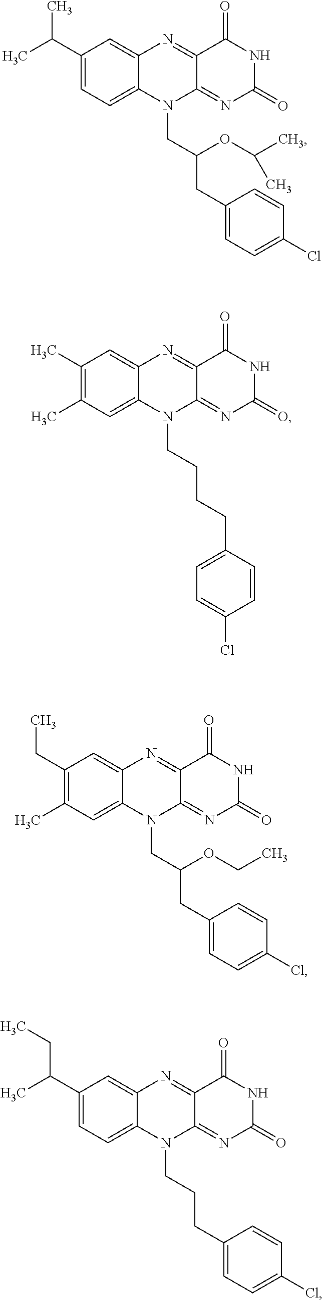 [SMILES: CC(C)OC(Cc1ccc(Cl)cc1)Cn1c2nc(=O)[nH]c(=O)c-2nc2cc(C(C)C)ccc21 ; Cc1cc2nc3c(=O)[nH]c(=O)nc-3n(CCCCc3ccc(Cl)cc3)c2cc1C ; CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1] 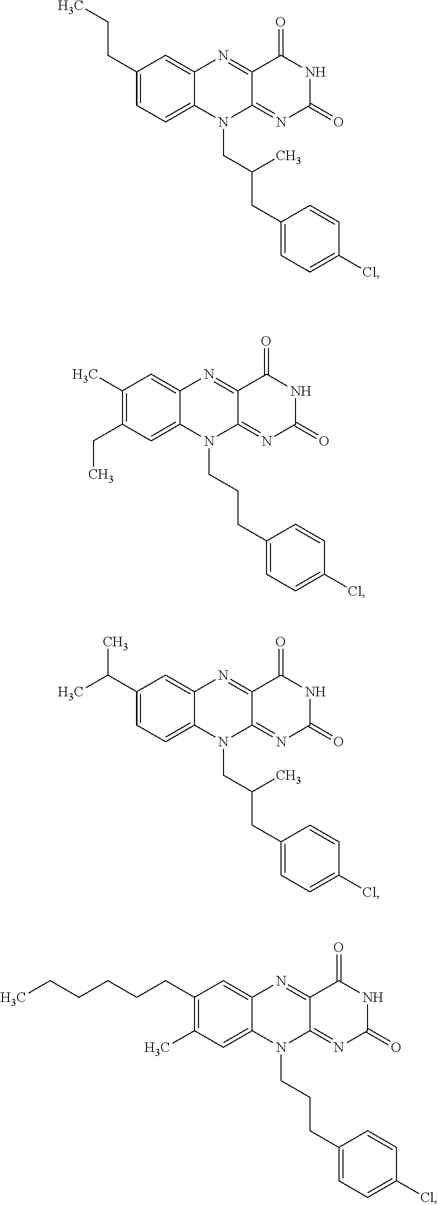 [SMILES: CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1 ; CC(Cc1ccc(Cl)cc1)Cn1c2nc(=O)[nH]c(=O)c-2nc2cc(C(C)C)ccc21 ; CCCCCCc1cc2nc3c(=O)[nH]c(=O)nc-3n(CCCc3ccc(Cl)cc3)c2cc1C] 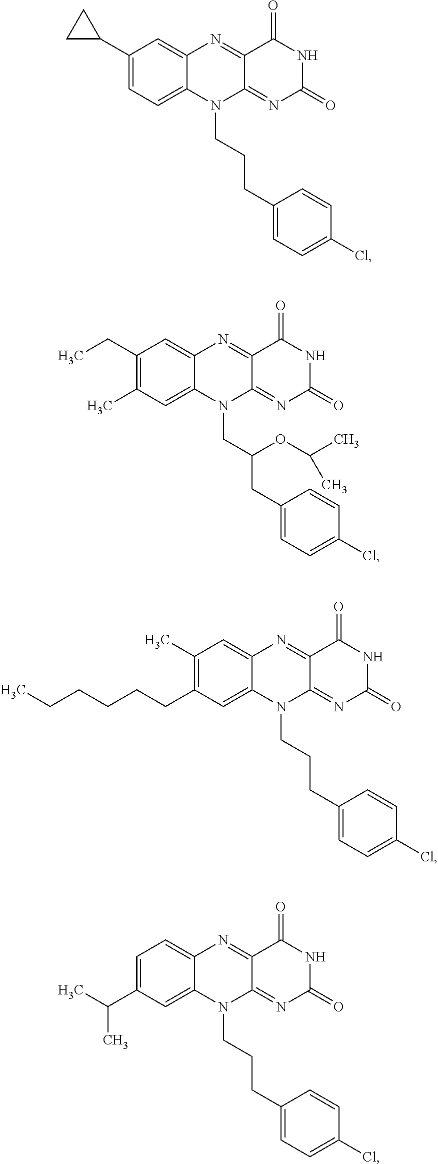 [SMILES: O=c1nc2n(CCCc3ccc(Cl)cc3)c3ccc(C4CC4)cc3nc-2c(=O)[nH]1 ; CCc1cc2nc3c(=O)[nH]c(=O)nc-3n(CC(Cc3ccc(Cl)cc3)OC(C)C)c2cc1C ; CCCCCCc1cc2c(cc1C)nc1c(=O)[nH]c(=O)nc-1n2CCCc1ccc(Cl)cc1 ; CC(C)c1ccc2nc3c(=O)[nH]c(=O)nc-3n(CCCc3ccc(Cl)cc3)c2c1] 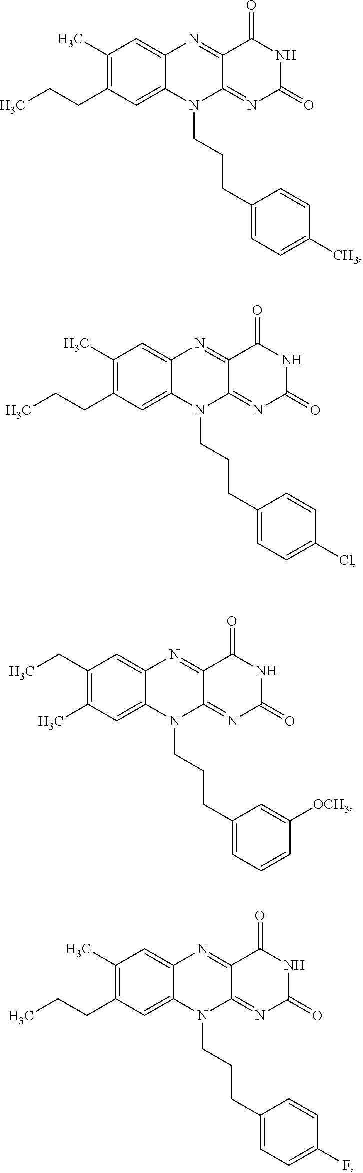 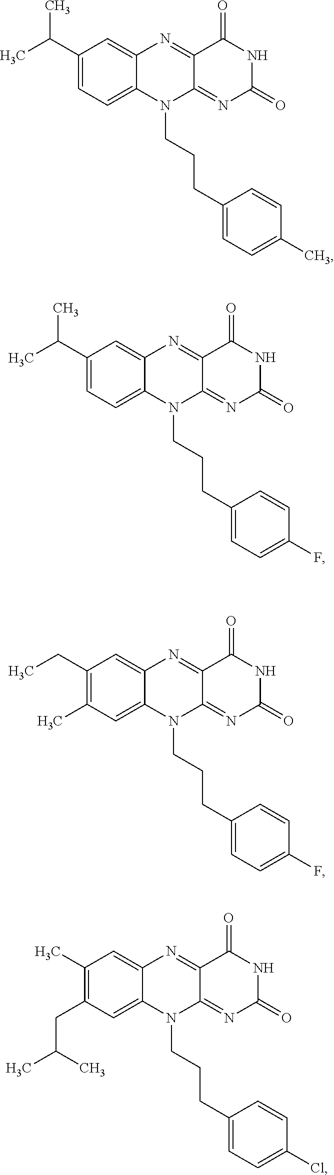 [SMILES: Cc1ccc(CCCn2c3nc(=O)[nH]c(=O)c-3nc3cc(C(C)C)ccc32)cc1 ; CC(C)c1ccc2c(c1)nc1c(=O)[nH]c(=O)nc-1n2CCCc1ccc(F)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C] 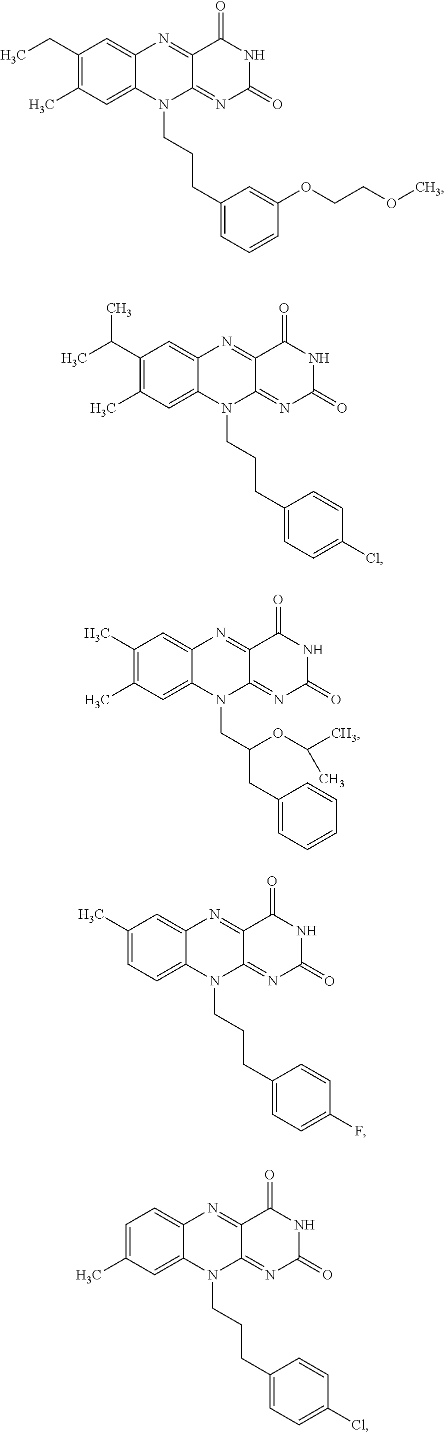 [SMILES: CCc1cc2nc3c(=O)[nH]c(=O)nc-3n(CCCc3cccc(OCCOC)c3)c2cc1C ; Cc1cc2c(cc1C(C)C)nc1c(=O)[nH]c(=O)nc-1n2CCCc1ccc(Cl)cc1 ; Cc1cc2nc3c(=O)[nH]c(=O)nc-3n(CC(Cc3ccccc3)OC(C)C)c2cc1C ; Cc1ccc2c(c1)nc1c(=O)[nH]c(=O)nc-1n2CCCc1ccc(F)cc1 ; Cc1ccc2nc3c(=O)[nH]c(=O)nc-3n(CCCc3ccc(Cl)cc3)c2c1] 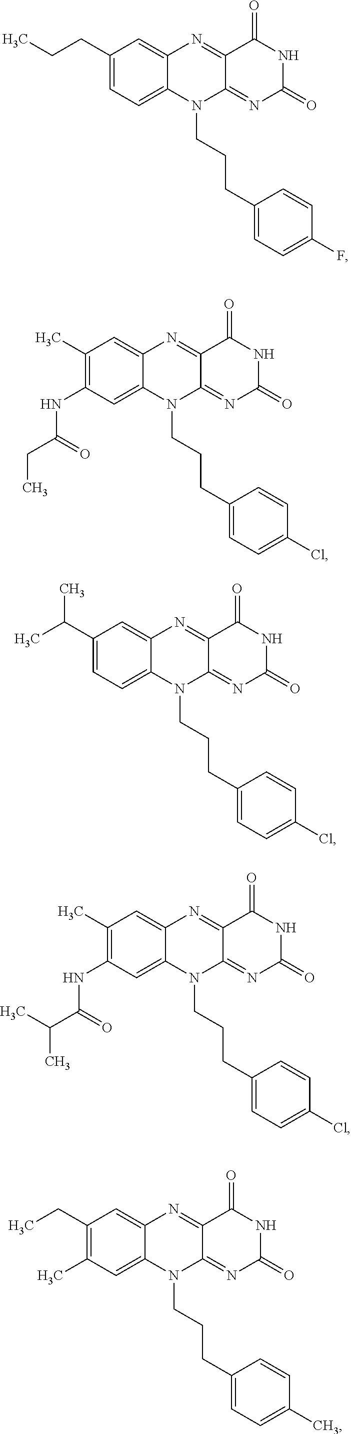 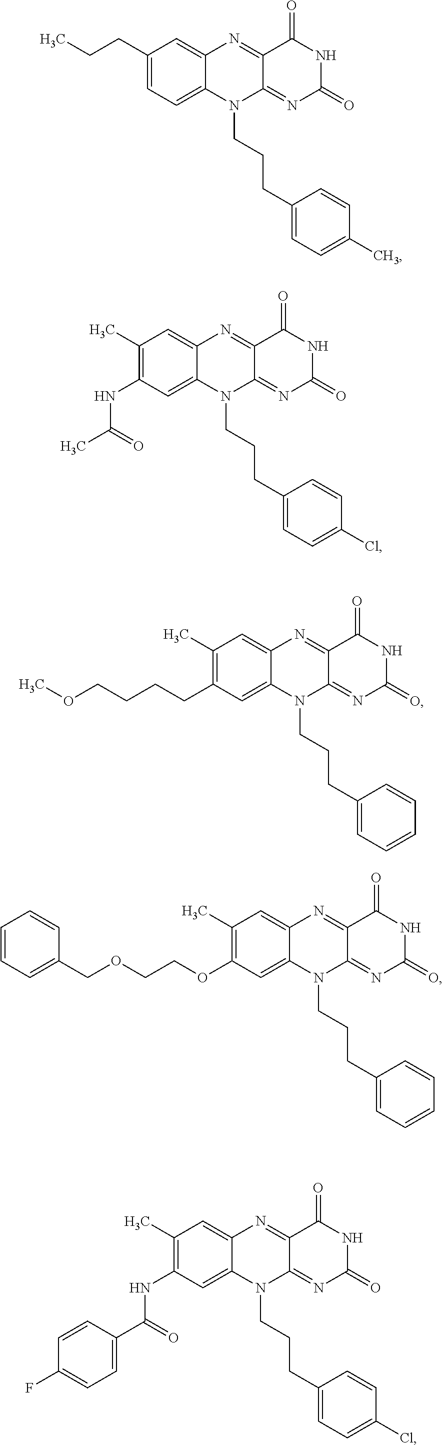 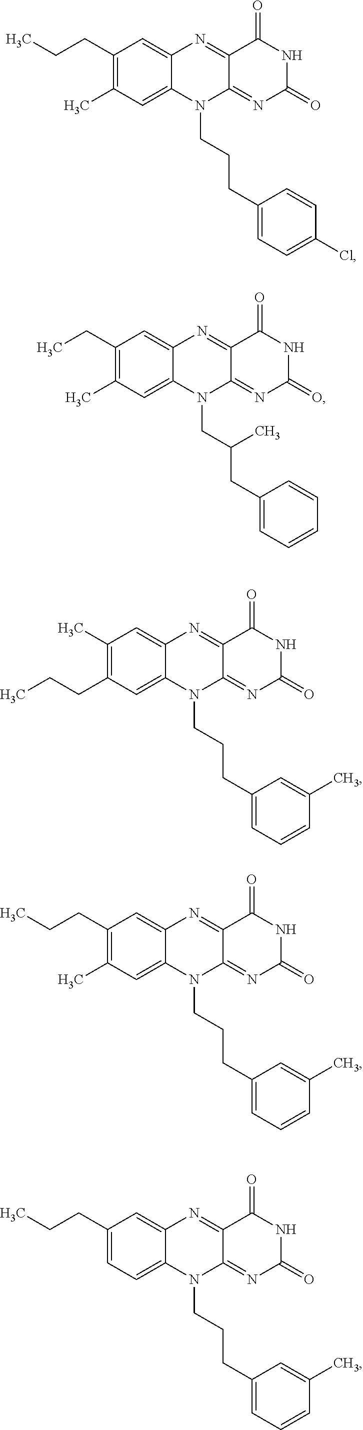 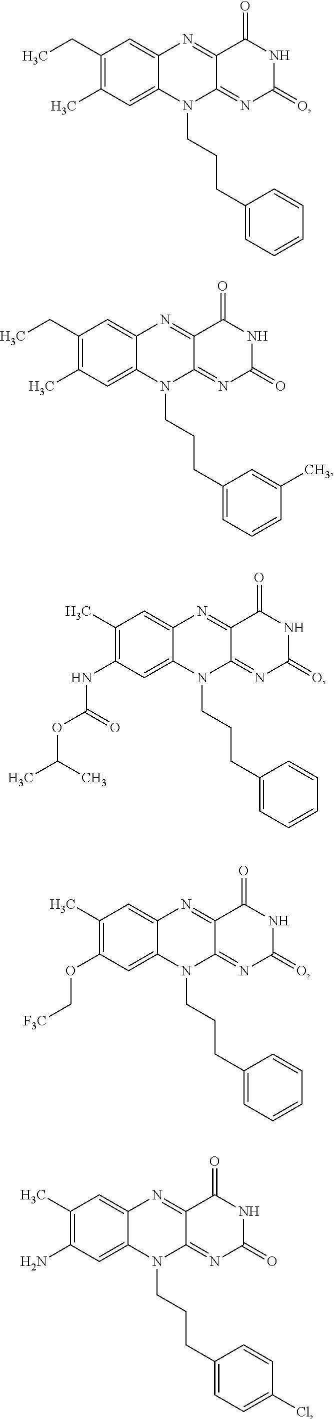 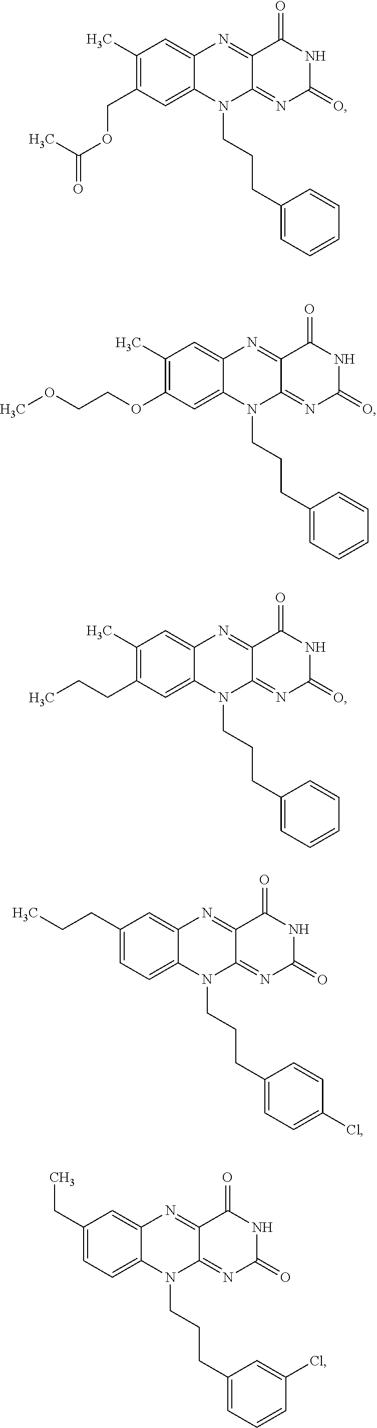 [SMILES: CC(=O)OCc1cc2c(cc1C)nc1c(=O)[nH]c(=O)nc-1n2CCCc1ccccc1 ; COCCOc1cc2c(cc1C)nc1c(=O)[nH]c(=O)nc-1n2CCCc1ccccc1 ; CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1 ; CCc1ccc2c(c1)nc1c(=O)[nH]c(=O)nc-1n2CCCc1cccc(Cl)c1] 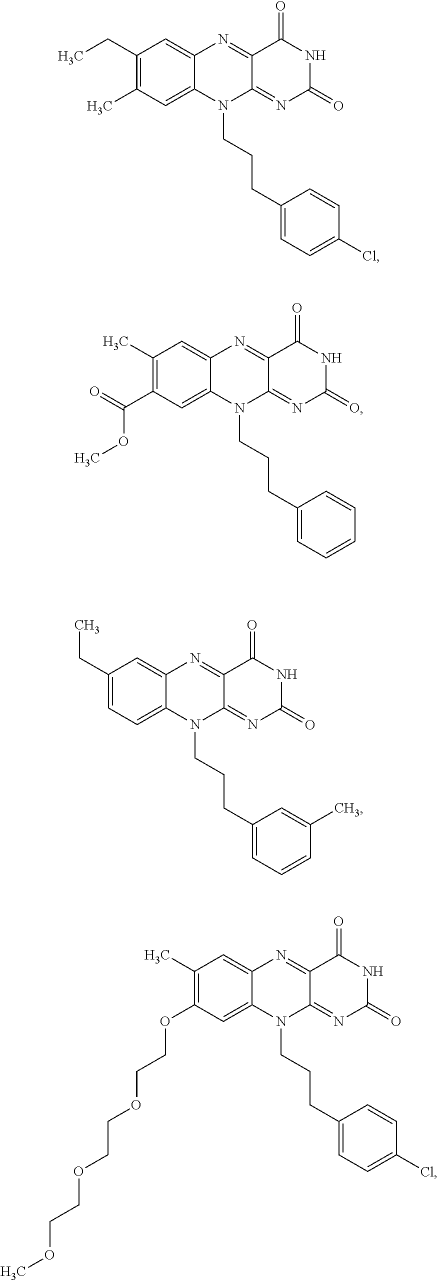 [SMILES: CCc1cc2nc3c(=O)[nH]c(=O)nc-3n(CCCc3ccc(Cl)cc3)c2cc1C ; COC(=O)c1cc2c(cc1C)nc1c(=O)[nH]c(=O)nc-1n2CCCc1ccccc1 ; CCc1ccc2c(c1)nc1c(=O)[nH]c(=O)nc-1n2CCCc1cccc(C)c1 ; COCCOCCOCCOc1cc2c(cc1C)nc1c(=O)[nH]c(=O)nc-1n2CCCc1ccc(Cl)cc1] 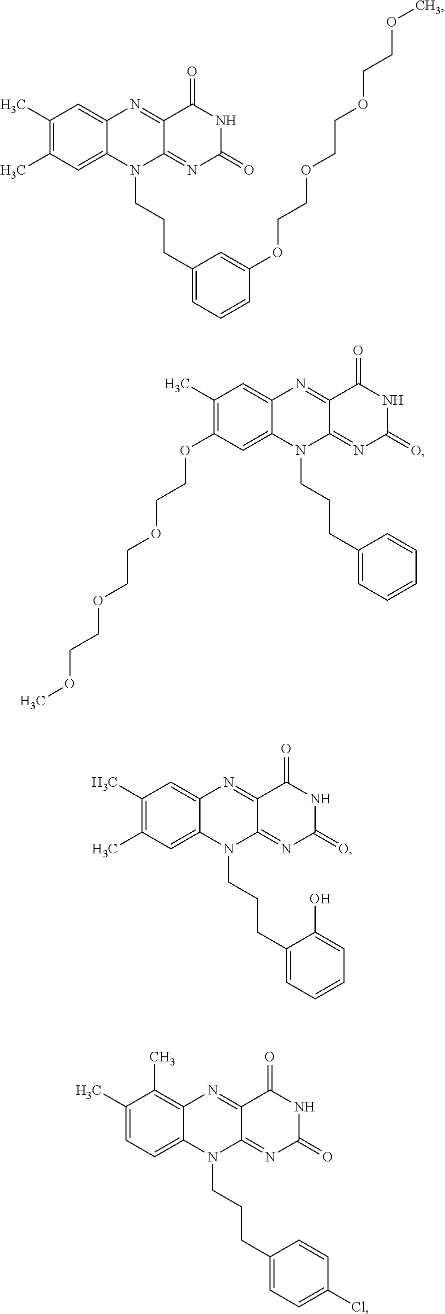 [SMILES: COCCOCCOCCOc1cccc(CCCn2c3nc(=O)[nH]c(=O)c-3nc3cc(C)c(C)cc32)c1 ; COCCOCCOCCOc1cc2c(cc1C)nc1c(=O)[nH]c(=O)nc-1n2CCCc1ccccc1 ; Cc1cc2nc3c(=O)[nH]c(=O)nc-3n(CCCc3ccccc3O)c2cc1C ; Cc1ccc2c(nc3c(=O)[nH]c(=O)nc-3n2CCCc2ccc(Cl)cc2)c1C] 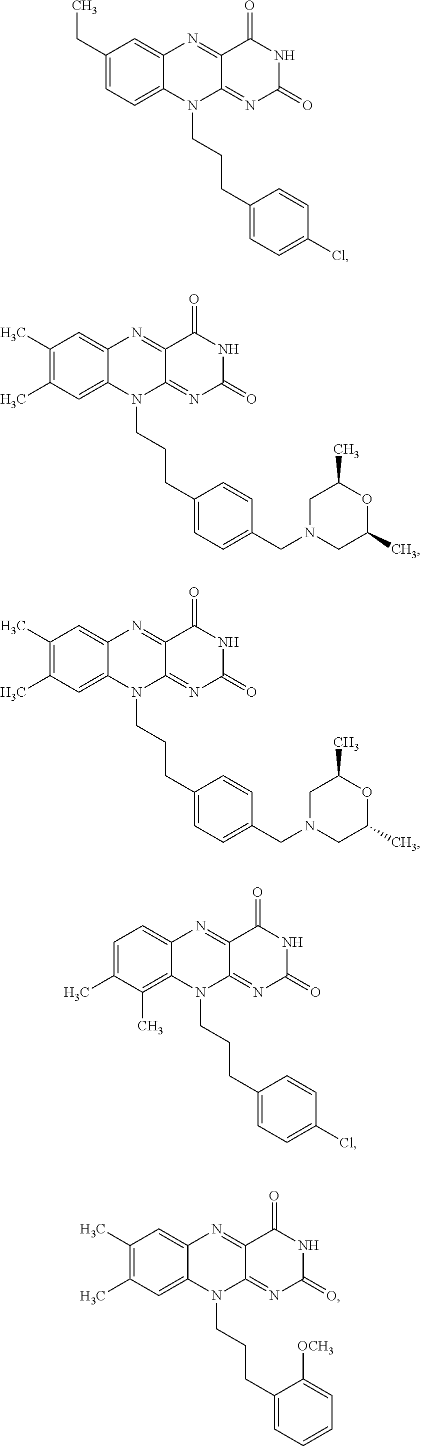 [SMILES: CCc1ccc2c(c1)nc1c(=O)[nH]c(=O)nc-1n2CCCc1ccc(Cl)cc1 ; Cc1cc2nc3c(=O)[nH]c(=O)nc-3n(CCCc3ccc(CN4C[C@@H](C)O[C@@H](C)C4)cc3)c2cc1C ; Cc1cc2nc3c(=O)[nH]c(=O)nc-3n(CCCc3ccc(CN4C[C@@H](C)O[C@H](C)C4)cc3)c2cc1C ; Cc1ccc2nc3c(=O)[nH]c(=O)nc-3n(CCCc3ccc(Cl)cc3)c2c1C ; COc1ccccc1CCCn1c2nc(=O)[nH]c(=O)c-2nc2cc(C)c(C)cc21] 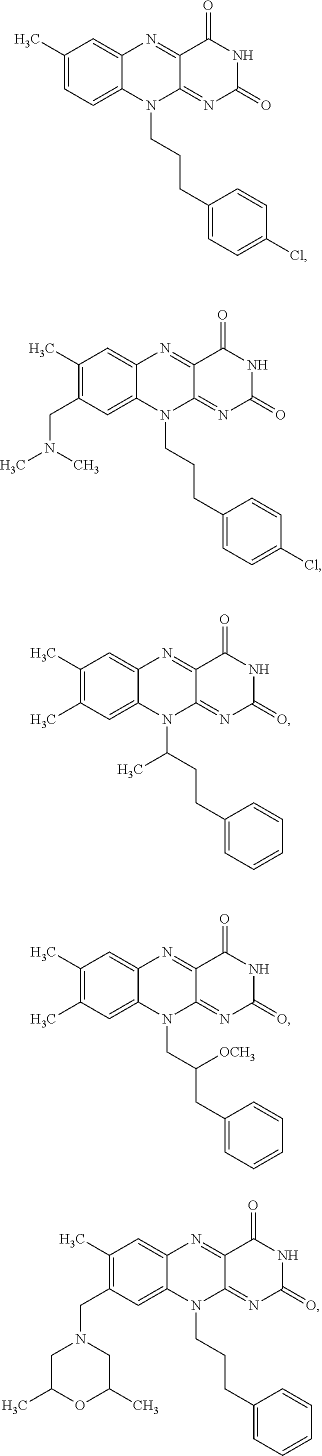 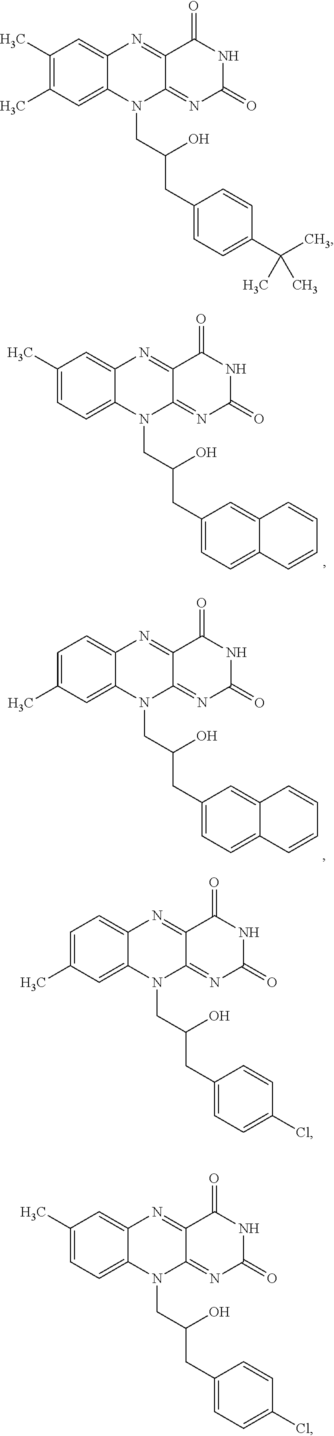 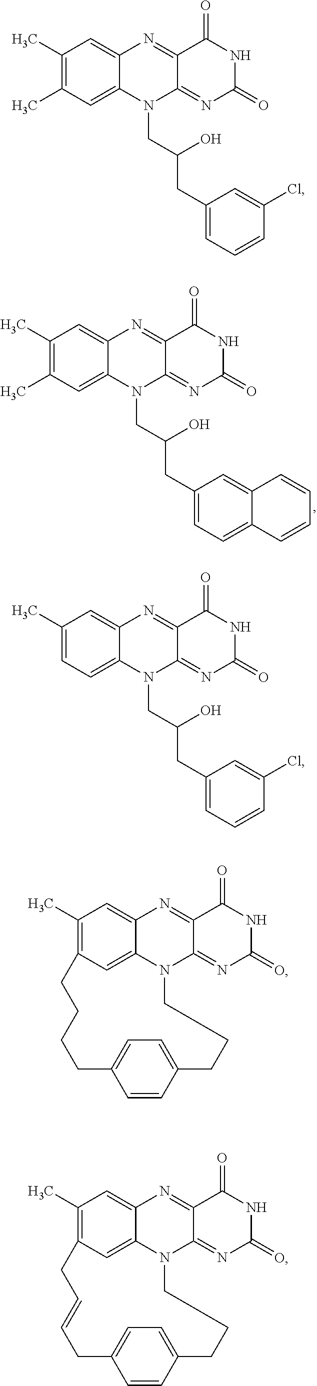 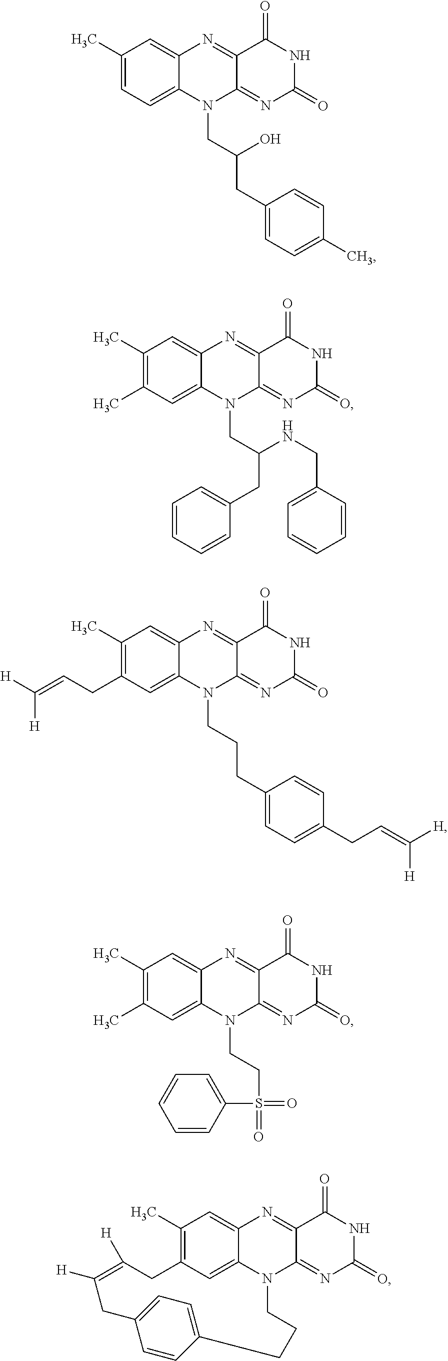 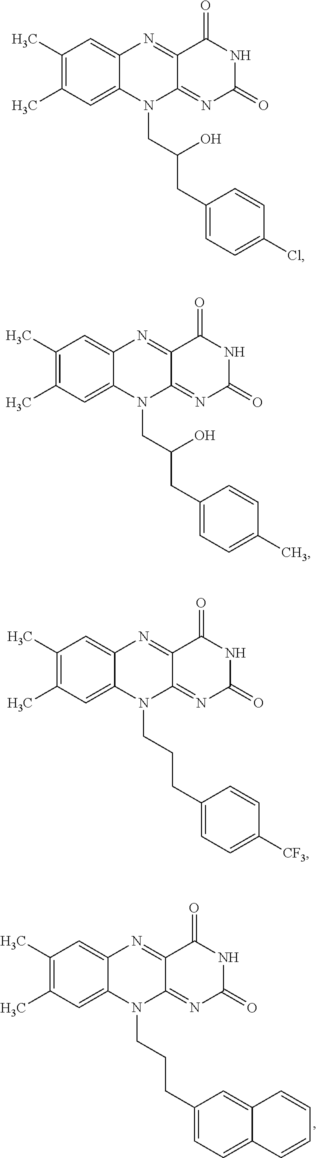 [SMILES: Cc1cc2nc3c(=O)[nH]c(=O)nc-3n(CC(O)Cc3ccc(Cl)cc3)c2cc1C ; Cc1ccc(CC(O)Cn2c3nc(=O)[nH]c(=O)c-3nc3cc(C)c(C)cc32)cc1 ; Cc1cc2nc3c(=O)[nH]c(=O)nc-3n(CCCc3ccc(C(F)(F)F)cc3)c2cc1C ; Cc1cc2nc3c(=O)[nH]c(=O)nc-3n(CCCc3ccc4ccccc4c3)c2cc1C] 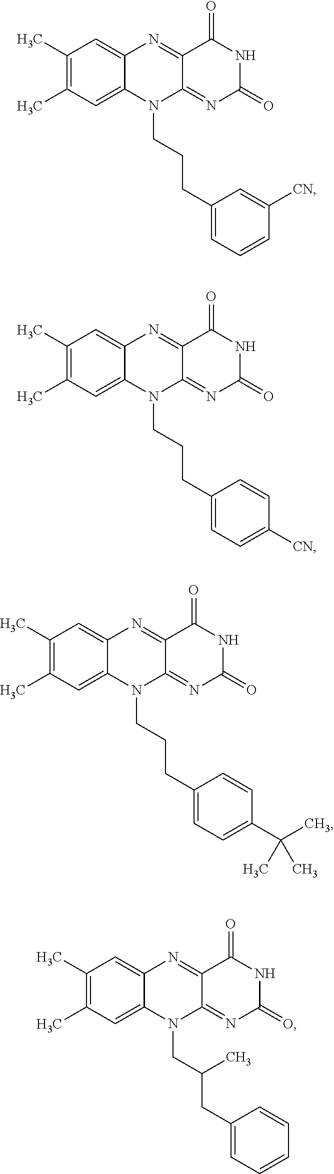 [SMILES: Cc1cc2nc3c(=O)[nH]c(=O)nc-3n(CCCc3cccc(C#N)c3)c2cc1C ; Cc1cc2nc3c(=O)[nH]c(=O)nc-3n(CCCc3ccc(C#N)cc3)c2cc1C ; Cc1cc2nc3c(=O)[nH]c(=O)nc-3n(CCCc3ccc(C(C)(C)C)cc3)c2cc1C ; Cc1cc2nc3c(=O)[nH]c(=O)nc-3n(CC(C)Cc3ccccc3)c2cc1C] 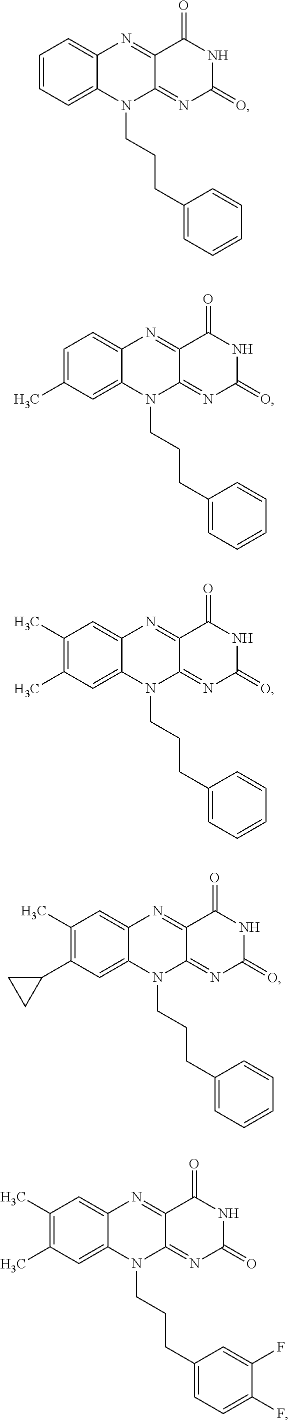 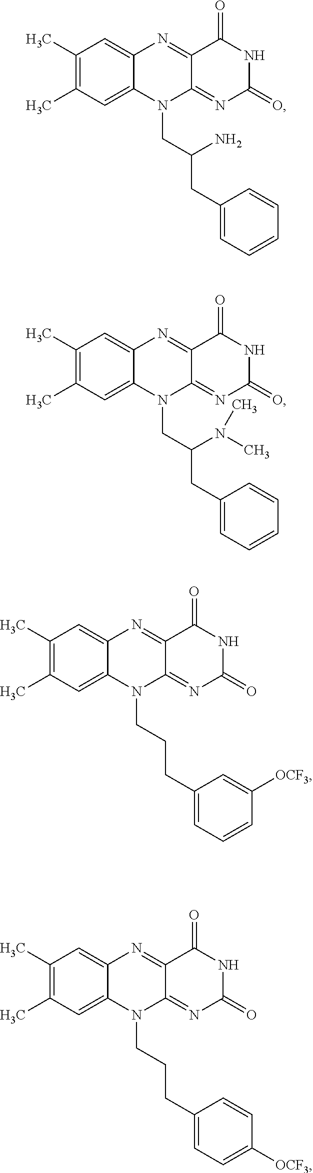 [SMILES: Cc1cc2nc3c(=O)[nH]c(=O)nc-3n(CC(N)Cc3ccccc3)c2cc1C ; Cc1cc2nc3c(=O)[nH]c(=O)nc-3n(CC(Cc3ccccc3)N(C)C)c2cc1C ; Cc1cc2nc3c(=O)[nH]c(=O)nc-3n(CCCc3cccc(OC(F)(F)F)c3)c2cc1C ; Cc1cc2nc3c(=O)[nH]c(=O)nc-3n(CCCc3ccc(OC(F)(F)F)cc3)c2cc1C] 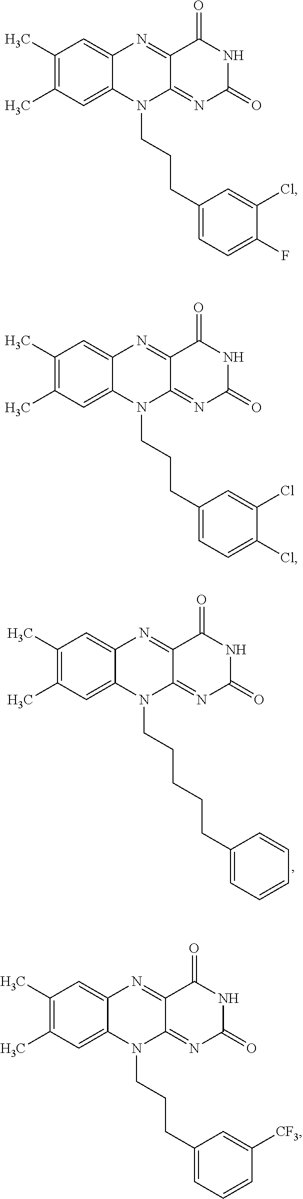 [SMILES: Cc1cc2nc3c(=O)[nH]c(=O)nc-3n(CCCc3ccc(F)c(Cl)c3)c2cc1C ; Cc1cc2nc3c(=O)[nH]c(=O)nc-3n(CCCc3ccc(Cl)c(Cl)c3)c2cc1C ; Cc1cc2nc3c(=O)[nH]c(=O)nc-3n(CCCCCc3ccccc3)c2cc1C ; Cc1cc2nc3c(=O)[nH]c(=O)nc-3n(CCCc3cccc(C(F)(F)F)c3)c2cc1C] 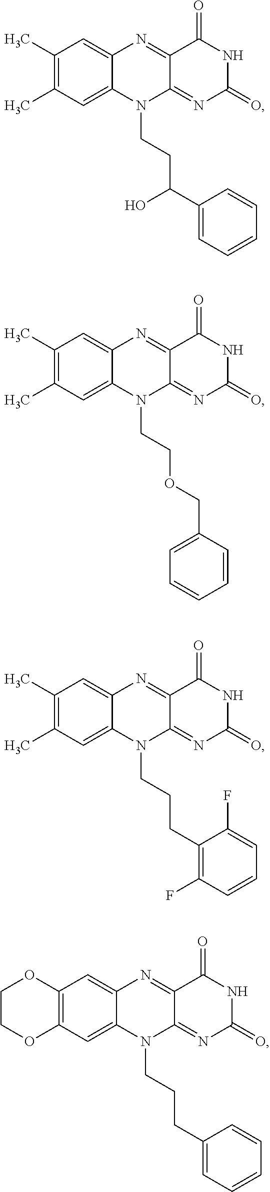 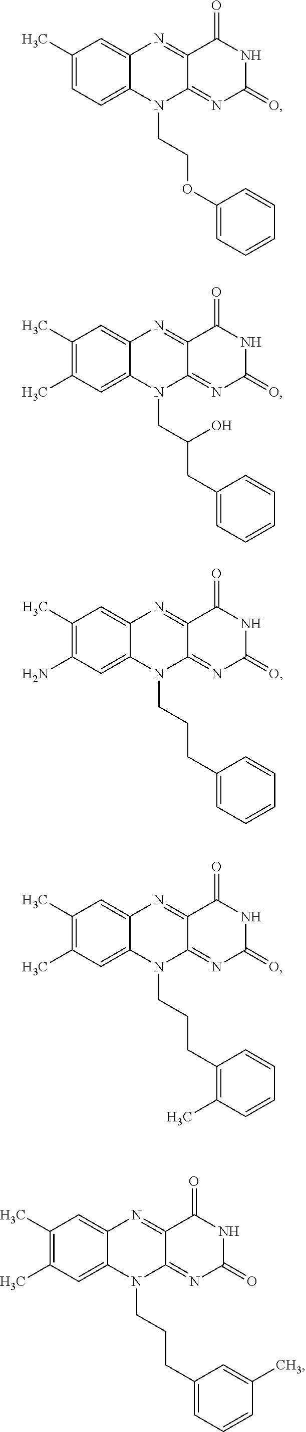 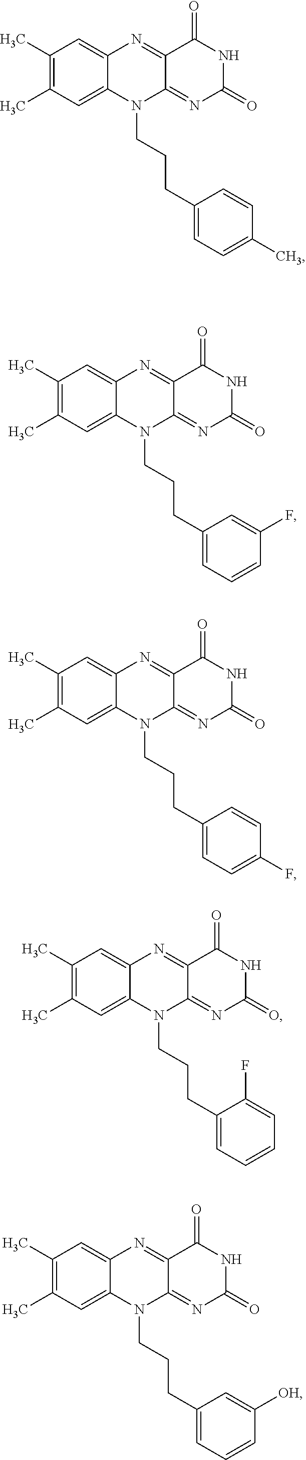 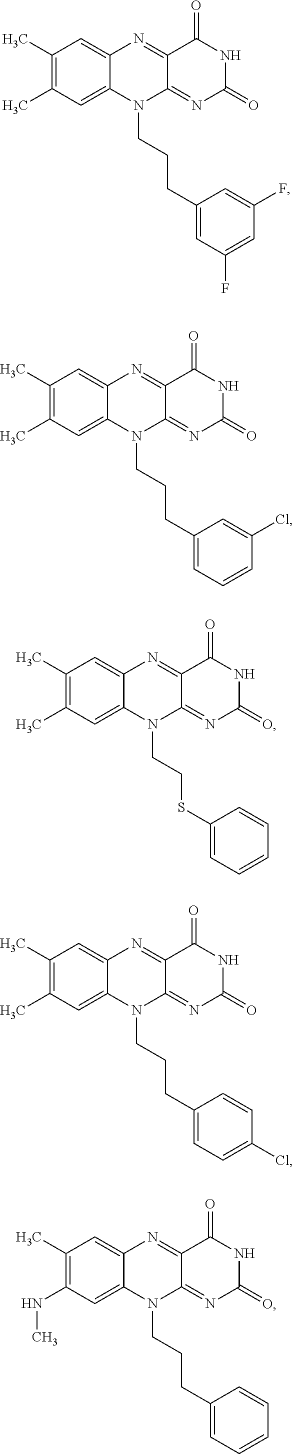 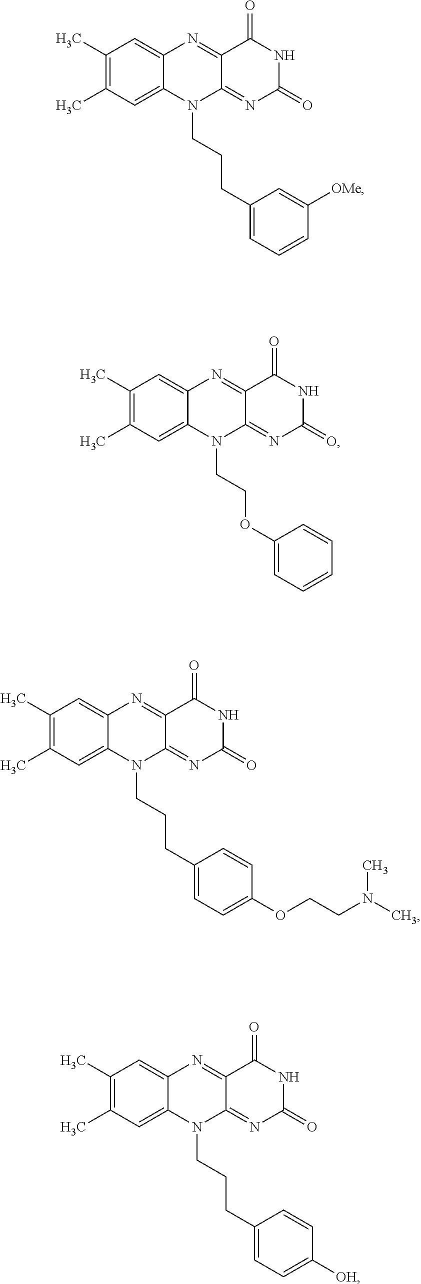 [SMILES: COc1cccc(CCCn2c3nc(=O)[nH]c(=O)c-3nc3cc(C)c(C)cc32)c1 ; Cc1cc2nc3c(=O)[nH]c(=O)nc-3n(CCOc3ccccc3)c2cc1C ; Cc1cc2nc3c(=O)[nH]c(=O)nc-3n(CCCc3ccc(OCCN(C)C)cc3)c2cc1C ; Cc1cc2nc3c(=O)[nH]c(=O)nc-3n(CCCc3ccc(O)cc3)c2cc1C] 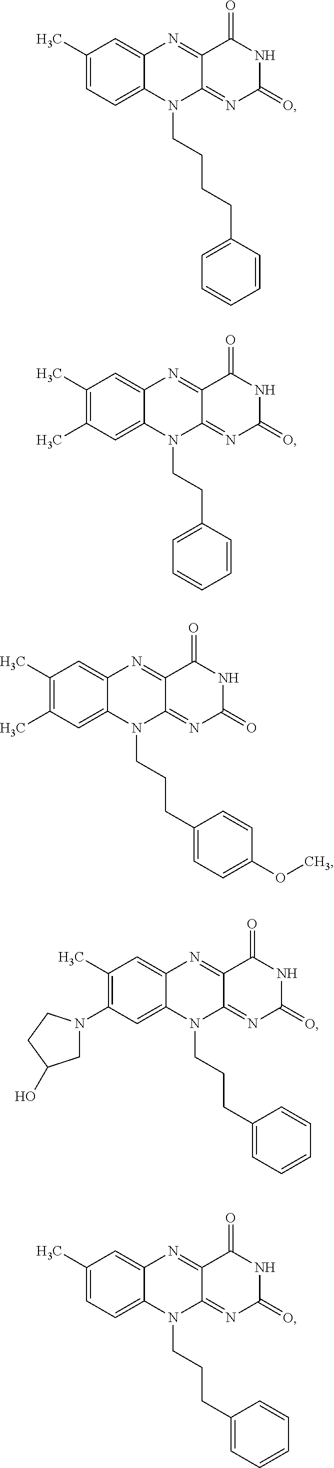 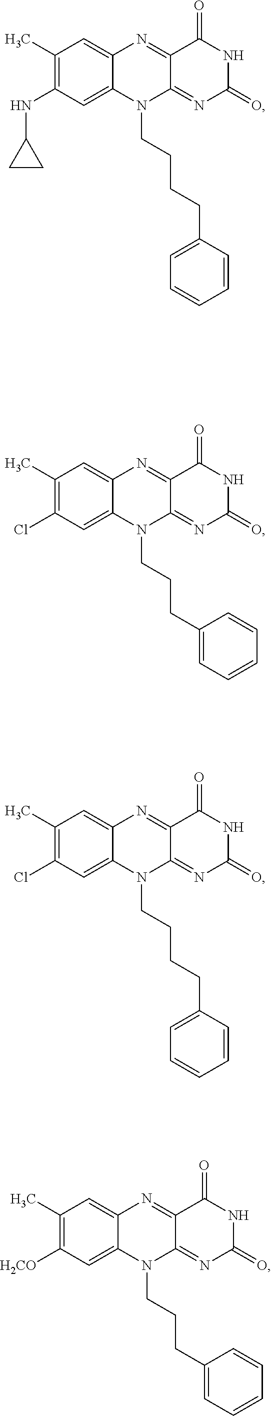 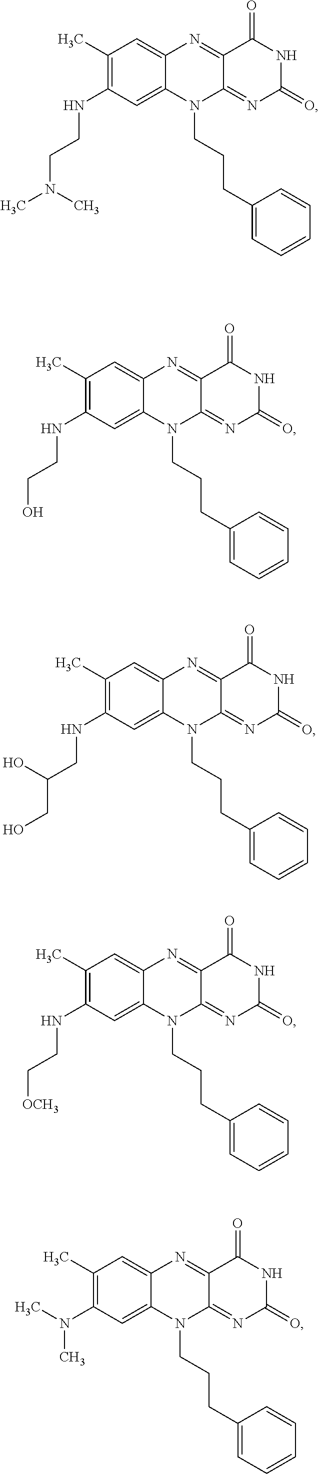 [SMILES: Cc1cc2nc3c(=O)[nH]c(=O)nc-3n(CCCc3ccccc3)c2cc1NCCN(C)C ; Cc1cc2nc3c(=O)[nH]c(=O)nc-3n(CCCc3ccccc3)c2cc1NCCO ; Cc1cc2nc3c(=O)[nH]c(=O)nc-3n(CCCc3ccccc3)c2cc1NCC(O)CO ; COCCNc1cc2c(cc1C)nc1c(=O)[nH]c(=O)nc-1n2CCCc1ccccc1 ; Cc1cc2nc3c(=O)[nH]c(=O)nc-3n(CCCc3ccccc3)c2cc1N(C)C] 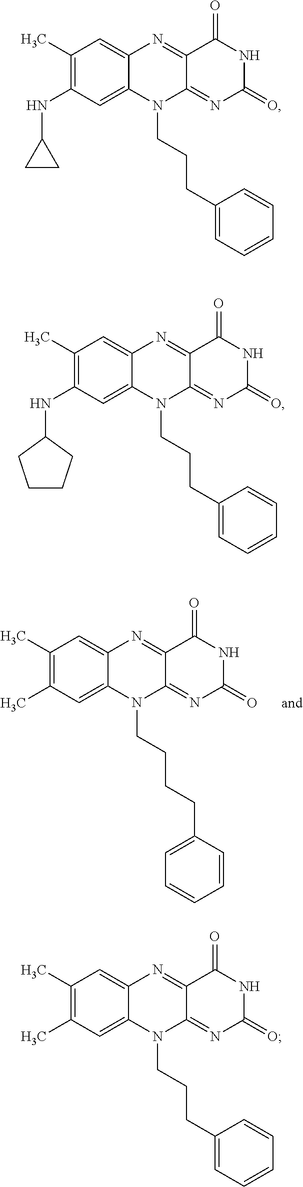
in free or pharmaceutically acceptable salt form.

17. The pharmaceutical composition according to any one of claims 10-16, wherein said compound is selected from any of the following 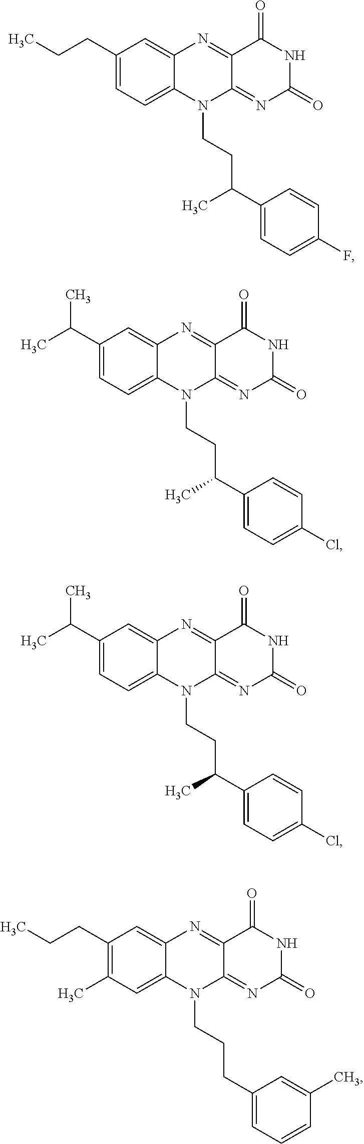 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCCc1cc2nc3c(=O)[nH]c(=O)nc-3n(CCCc3cccc(C)c3)c2cc1C] 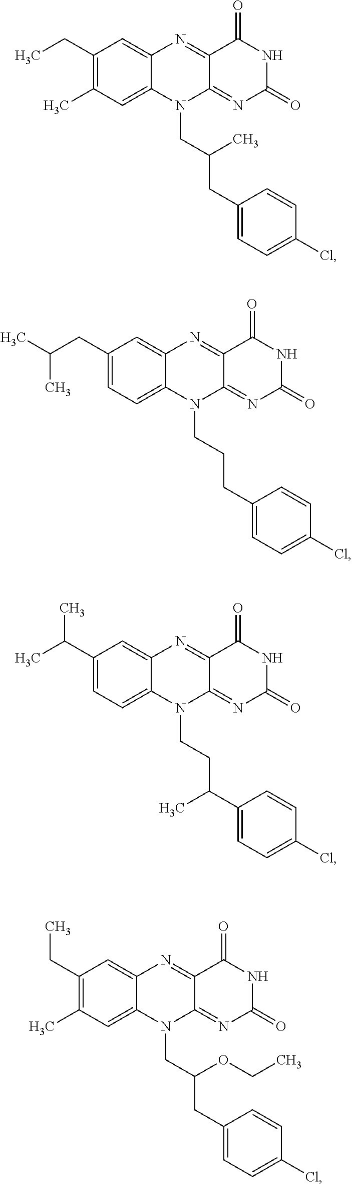 [SMILES: CCc1cc2nc3c(=O)[nH]c(=O)nc-3n(CC(C)Cc3ccc(Cl)cc3)c2cc1C ; CC(C)Cc1ccc2c(c1)nc1c(=O)[nH]c(=O)nc-1n2CCCc1ccc(Cl)cc1 ; CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21] 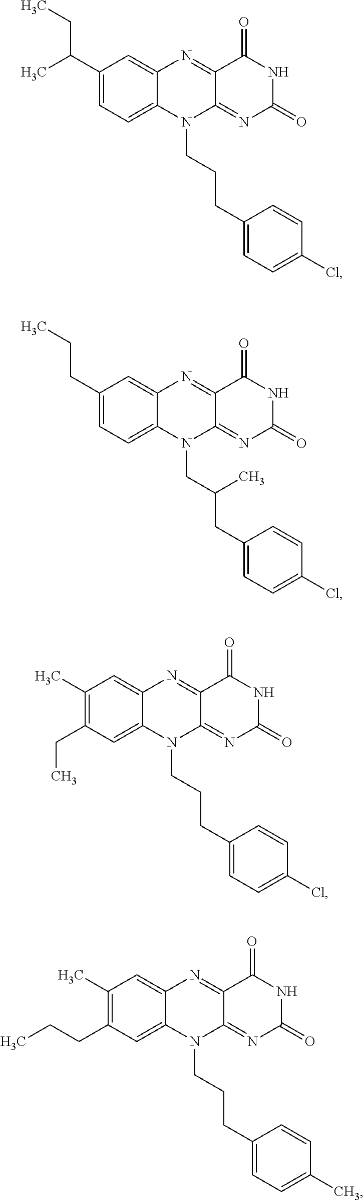 [SMILES: CCC(C)c1ccc2c(c1)nc1c(=O)[nH]c(=O)nc-1n2CCCc1ccc(Cl)cc1 ; CCCc1ccc2c(c1)nc1c(=O)[nH]c(=O)nc-1n2CC(C)Cc1ccc(Cl)cc1 ; CCc1cc2c(cc1C)nc1c(=O)[nH]c(=O)nc-1n2CCCc1ccc(Cl)cc1 ; CCCc1cc2c(cc1C)nc1c(=O)[nH]c(=O)nc-1n2CCCc1ccc(C)cc1] 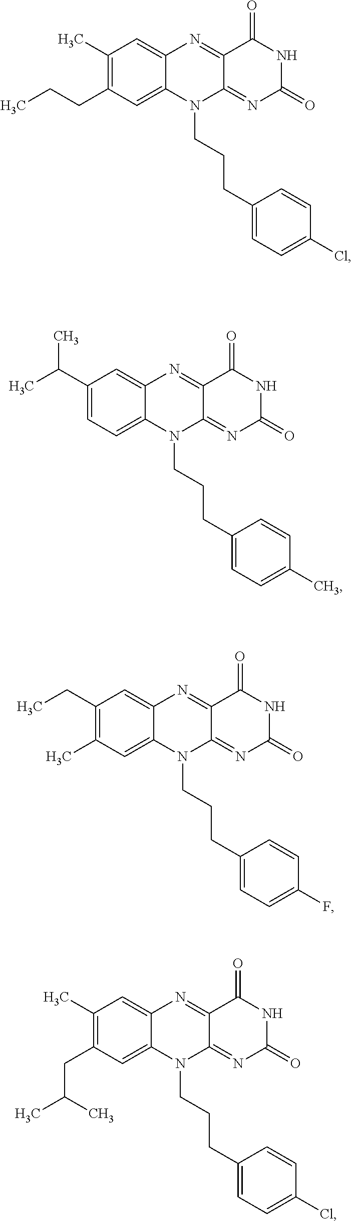 [SMILES: CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C ; Cc1cc2nc3c(=O)[nH]c(=O)nc-3n(CCCc3ccc(Cl)cc3)c2cc1CC(C)C] 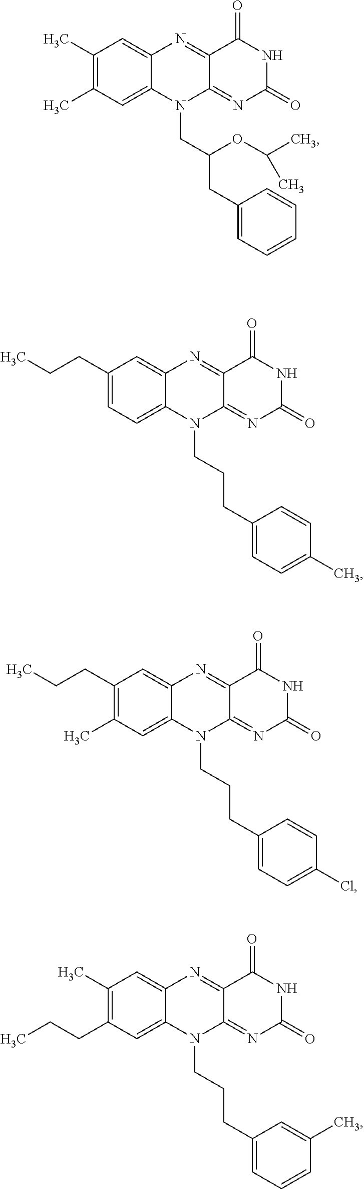 [SMILES: Cc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCCc1ccc(C)cc1 ; CCCc1cc2nc3c(=O)[nH]c(=O)nc-3n(CCCc3ccc(Cl)cc3)c2cc1C ; CCCc1cc2c(cc1C)nc1c(=O)[nH]c(=O)nc-1n2CCCc1cccc(C)c1] 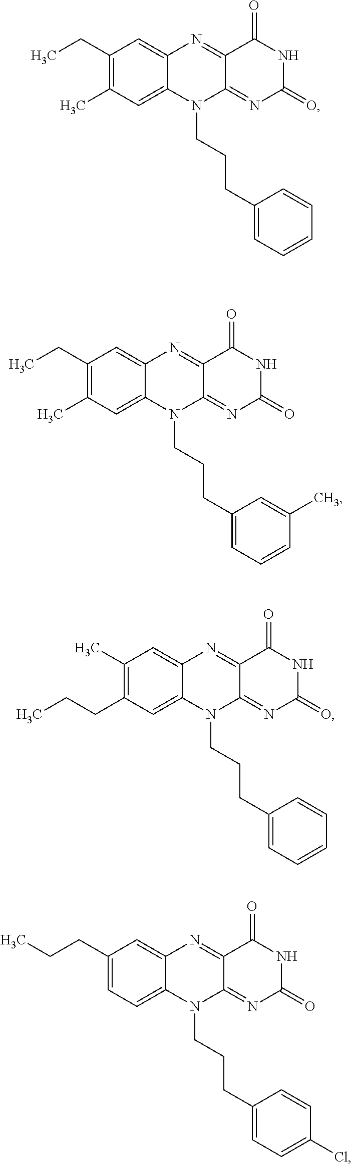 [SMILES: CCc1cc2nc3c(=O)[nH]c(=O)nc-3n(CCCc3ccccc3)c2cc1C ; CCc1cc2nc3c(=O)[nH]c(=O)nc-3n(CCCc3cccc(C)c3)c2cc1C ; CCCc1cc2c(cc1C)nc1c(=O)[nH]c(=O)nc-1n2CCCc1ccccc1 ; CCCc1ccc2c(c1)nc1c(=O)[nH]c(=O)nc-1n2CCCc1ccc(Cl)cc1] 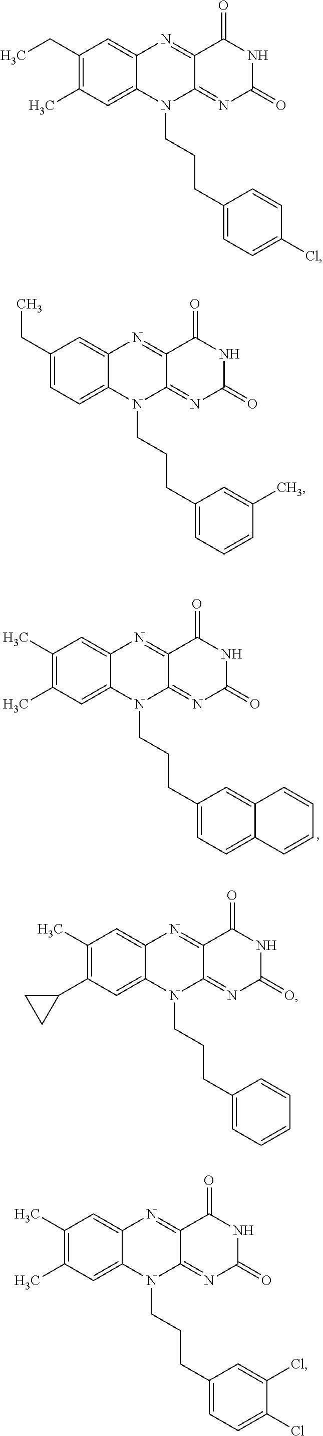 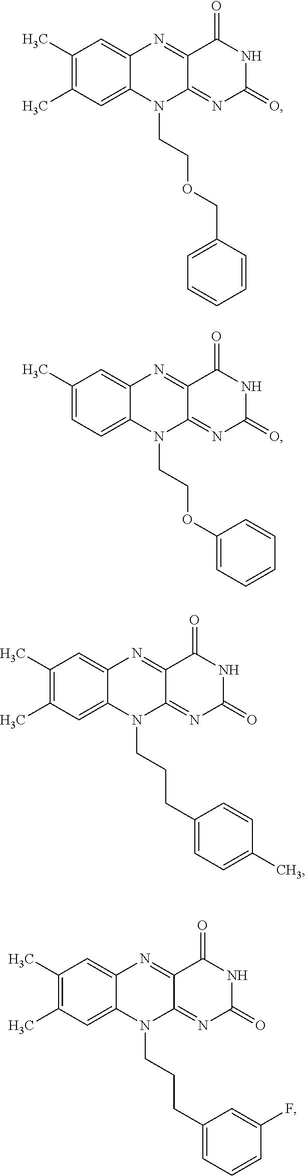 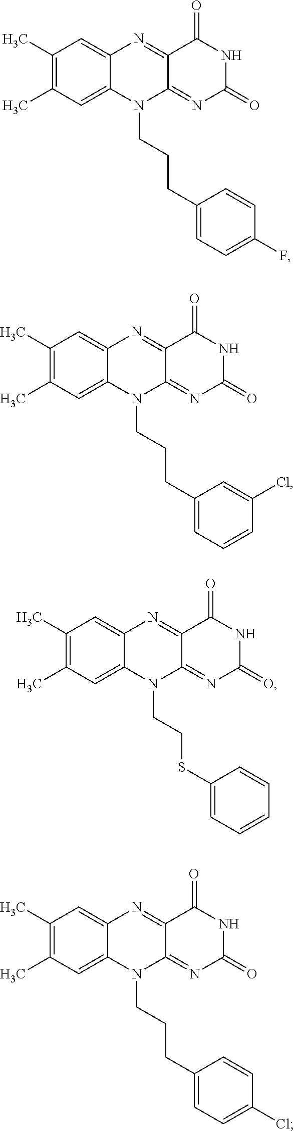 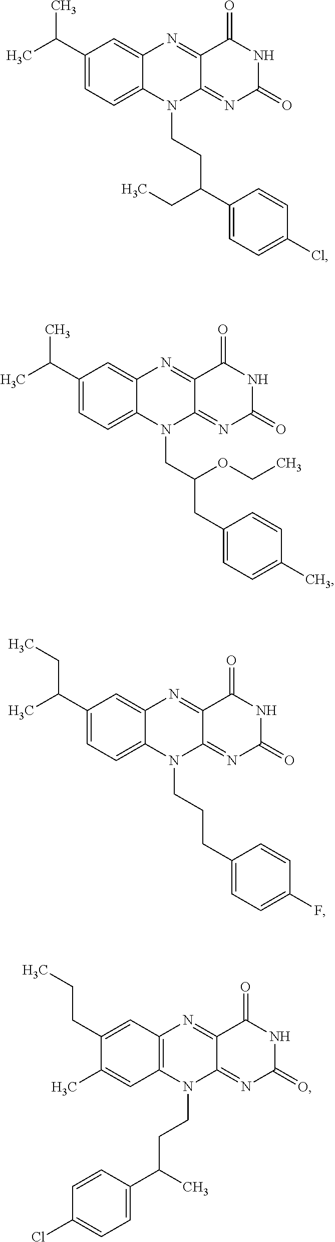 [SMILES: CCC(CCn1c2nc(=O)[nH]c(=O)c-2nc2cc(C(C)C)ccc21)c1ccc(Cl)cc1 ; CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C] 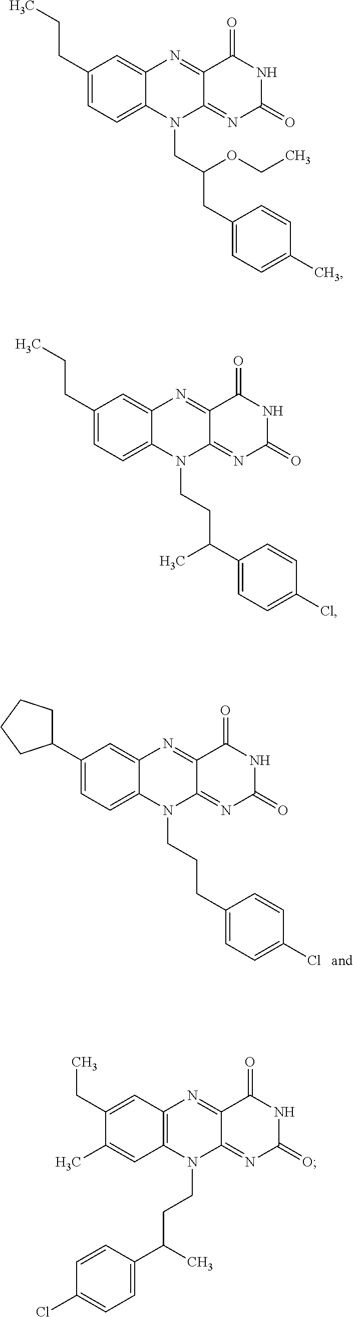 [SMILES: CCCc1ccc2c(c1)nc1c(=O)[nH]c(=O)nc-1n2CC(Cc1ccc(C)cc1)OCC ; CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; "and" ; CCc1cc2nc3c(=O)[nH]c(=O)nc-3n(CCC(C)c3ccc(Cl)cc3)c2cc1C]
in free or pharmaceutically acceptable salt form.

18. The pharmaceutical composition according to any one of claims 10-17, wherein said compound is selected from any of the following 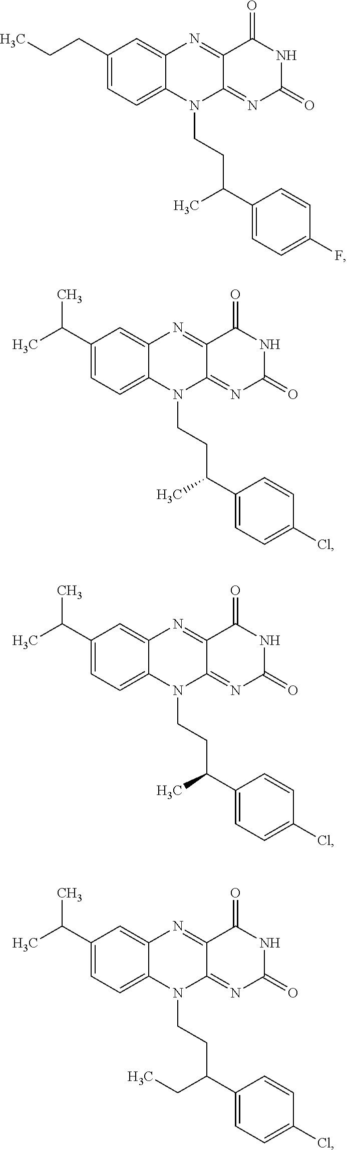 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1] 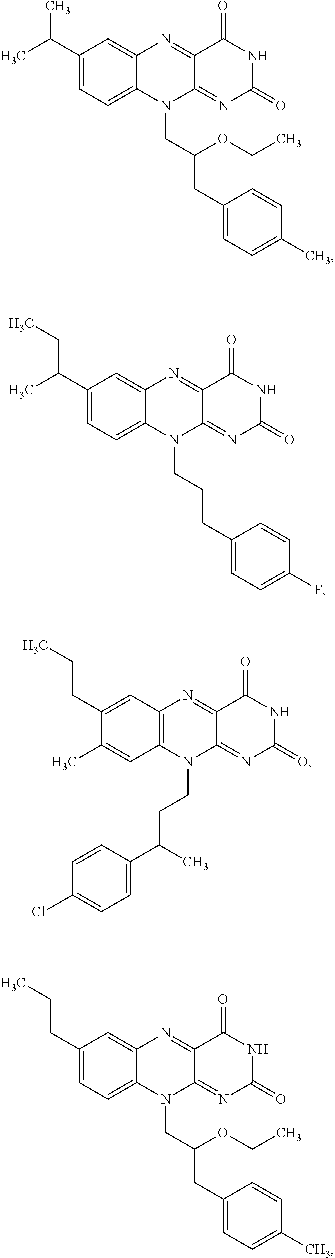 [SMILES: CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCc1cc2nc3c(=O)[nH]c(=O)nc-3n(CCC(C)c3ccc(Cl)cc3)c2cc1C ; CCCc1ccc2c(c1)nc1c(=O)[nH]c(=O)nc-1n2CC(Cc1ccc(C)cc1)OCC] 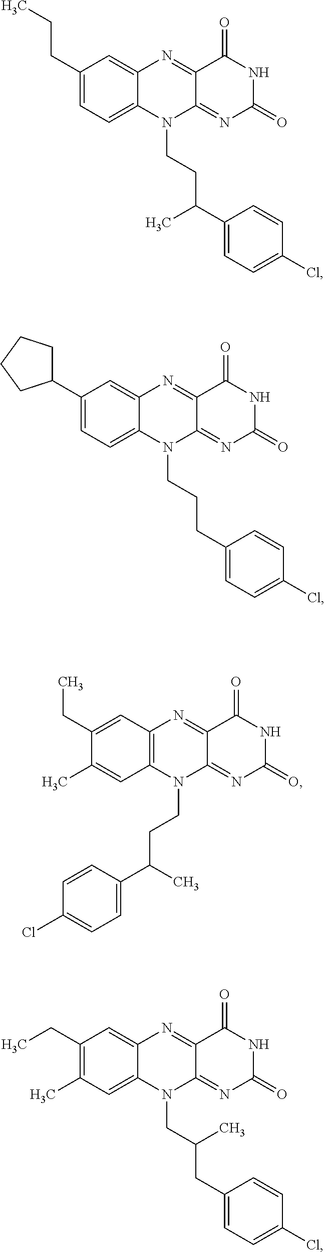 [SMILES: CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C ; CCc1cc2nc3c(=O)[nH]c(=O)nc-3n(CC(C)Cc3ccc(Cl)cc3)c2cc1C] 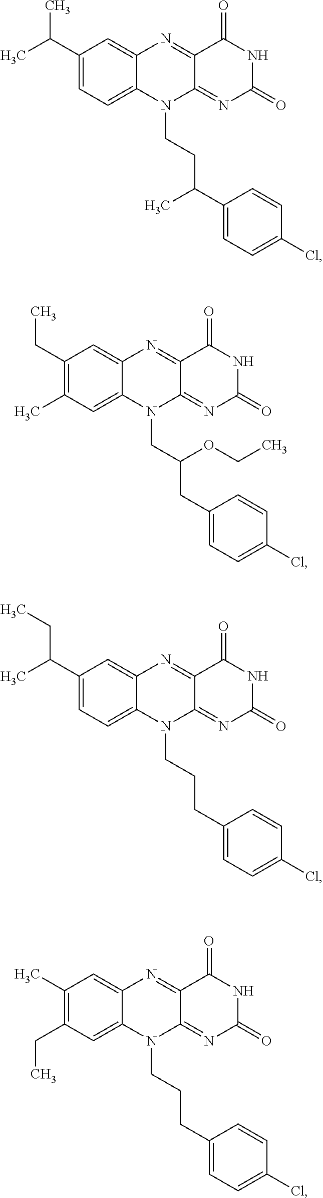 [SMILES: CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1 ; CCc1cc2c(cc1C)nc1c(=O)[nH]c(=O)nc-1n2CCCc1ccc(Cl)cc1] 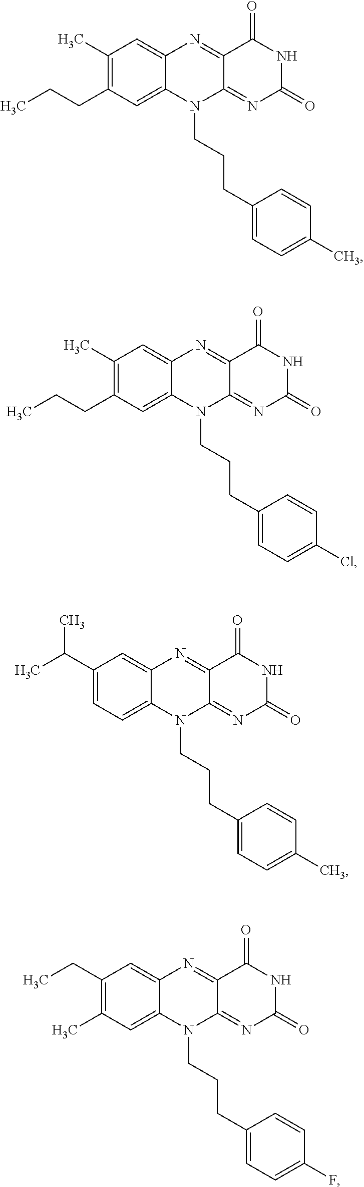 [SMILES: CCCc1cc2c(cc1C)nc1c(=O)[nH]c(=O)nc-1n2CCCc1ccc(C)cc1 ; CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C] 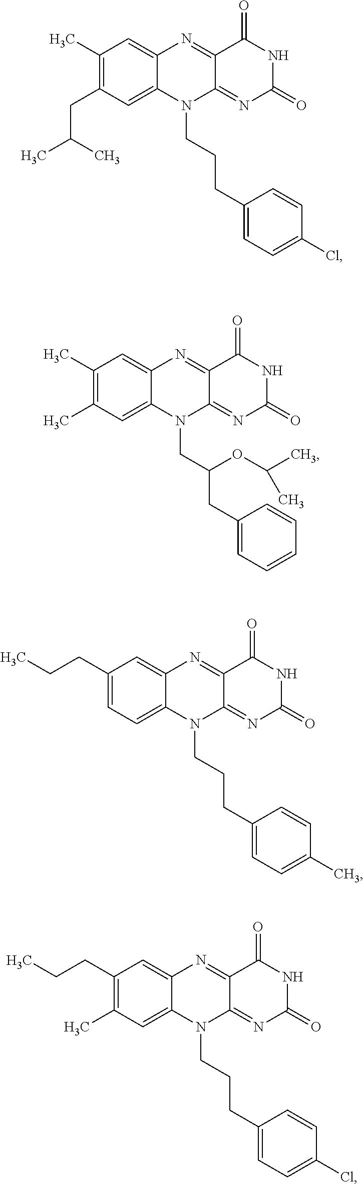 [SMILES: Cc1cc2nc3c(=O)[nH]c(=O)nc-3n(CCCc3ccc(Cl)cc3)c2cc1CC(C)C ; Cc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCCc1ccc(C)cc1 ; CCCc1cc2nc3c(=O)[nH]c(=O)nc-3n(CCCc3ccc(Cl)cc3)c2cc1C] 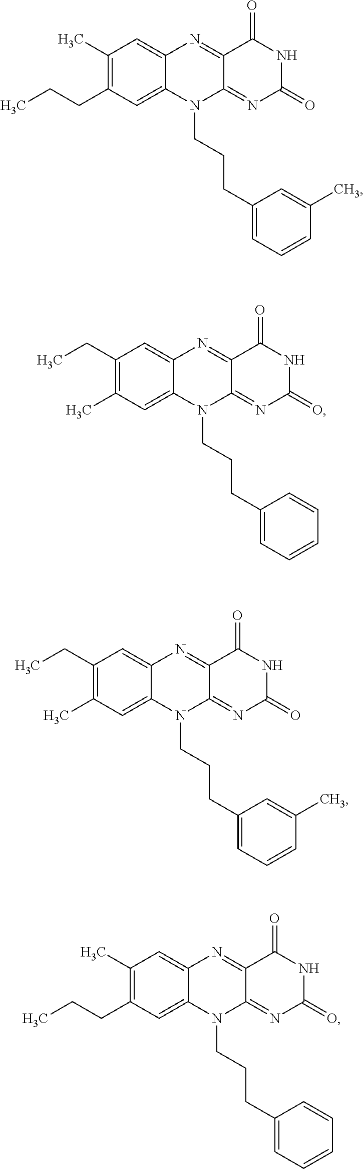 [SMILES: CCCc1cc2c(cc1C)nc1c(=O)[nH]c(=O)nc-1n2CCCc1cccc(C)c1 ; CCc1cc2nc3c(=O)[nH]c(=O)nc-3n(CCCc3ccccc3)c2cc1C ; CCc1cc2nc3c(=O)[nH]c(=O)nc-3n(CCCc3cccc(C)c3)c2cc1C ; CCCc1cc2c(cc1C)nc1c(=O)[nH]c(=O)nc-1n2CCCc1ccccc1] 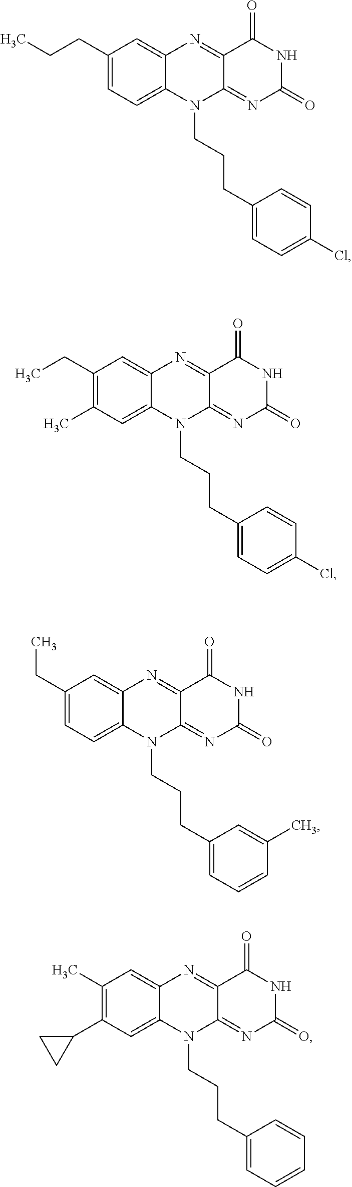 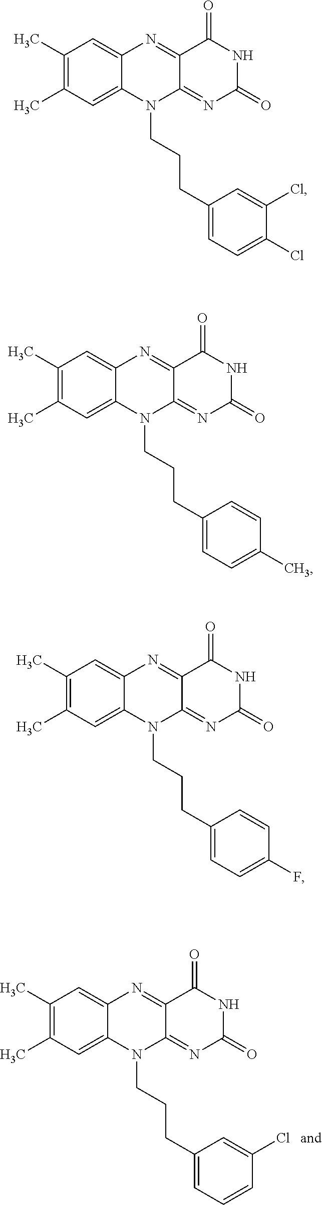 [SMILES: Cc1cc2nc3c(=O)[nH]c(=O)nc-3n(CCCc3ccc(Cl)c(Cl)c3)c2cc1C ; Cc1ccc(CCCn2c3nc(=O)[nH]c(=O)c-3nc3cc(C)c(C)cc32)cc1 ; Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(Cl)c3)c2cc1C ; "and"] 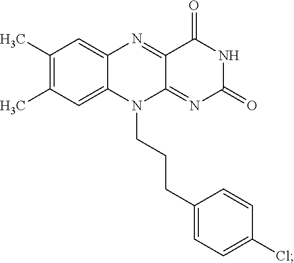 [SMILES: Cc1cc2nc3c(=O)[nH]c(=O)nc-3n(CCCc3ccc(Cl)cc3)c2cc1C]
in free or pharmaceutically acceptable salt form.

19. A method for the treatment or prophylaxis of a bacterial infection comprising administering to a patient in need of such treatment an effective amount of a compound according to any one of claims 10-18.

20. The method according to any one of claim 19, wherein the infection is a Gram-positive or Gram-negative bacterial infection.

21. The method according to any one of claims 19-20, wherein the bacterial infection is selected from a group consisting of Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and Borrelia burgdorferi.

22. The method according to any one of claims 19-21, wherein the bacterial infection is a C. difficile infection.

23. The method according to claim 22, wherein the compound is selected from any of the following: 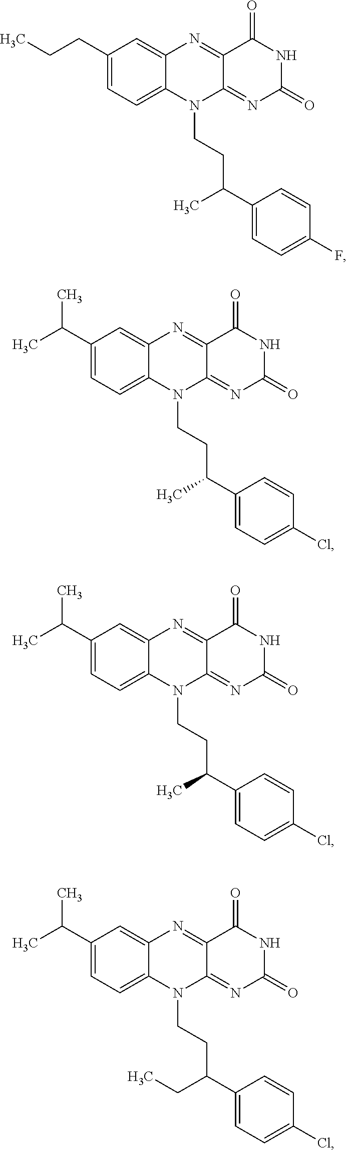 [SMILES: CCCc1ccc2c(c1)nc1c(=O)[nH]c(=O)nc-1n2CCC(C)c1ccc(F)cc1 ; CC(C)c1ccc2c(c1)nc1c(=O)[nH]c(=O)nc-1n2CC[C@@H](C)c1ccc(Cl)cc1 ; CC(C)c1ccc2c(c1)nc1c(=O)[nH]c(=O)nc-1n2CC[C@H](C)c1ccc(Cl)cc1 ; CCC(CCn1c2nc(=O)[nH]c(=O)c-2nc2cc(C(C)C)ccc21)c1ccc(Cl)cc1] 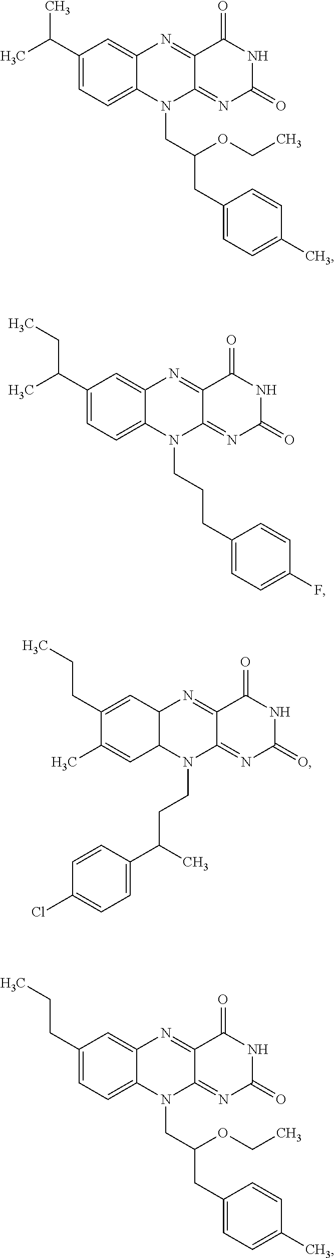 [SMILES: CCOC(Cc1ccc(C)cc1)Cn1c2nc(=O)[nH]c(=O)c-2nc2cc(C(C)C)ccc21 ; CCC(C)c1ccc2c(c1)nc1c(=O)[nH]c(=O)nc-1n2CCCc1ccc(F)cc1 ; CCCC1=CC2N=C3C(=O)NC(=O)N=C3N(CCC(C)c3ccc(Cl)cc3)C2C=C1C ; CCCc1ccc2c(c1)nc1c(=O)[nH]c(=O)nc-1n2CC(Cc1ccc(C)cc1)OCC] 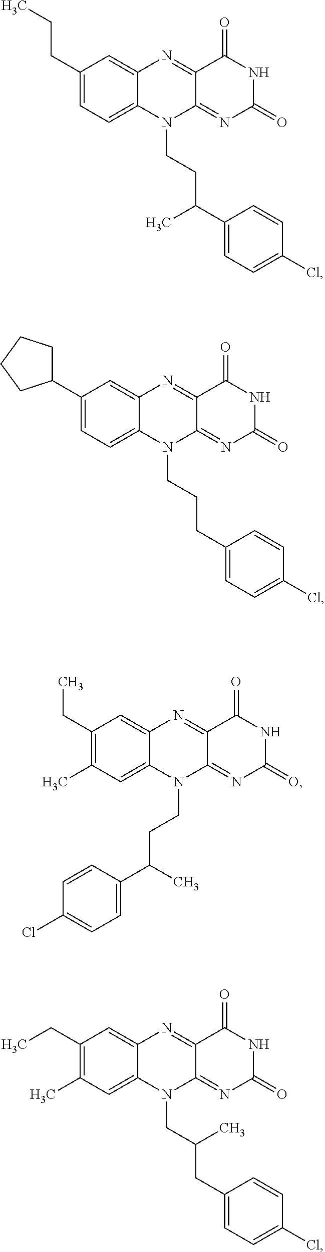 [SMILES: CCCc1ccc2c(c1)nc1c(=O)[nH]c(=O)nc-1n2CCC(C)c1ccc(Cl)cc1 ; O=c1nc2n(CCCc3ccc(Cl)cc3)c3ccc(C4CCCC4)cc3nc-2c(=O)[nH]1 ; CCc1cc2nc3c(=O)[nH]c(=O)nc-3n(CCC(C)c3ccc(Cl)cc3)c2cc1C ; CCc1cc2nc3c(=O)[nH]c(=O)nc-3n(CC(C)Cc3ccc(Cl)cc3)c2cc1C] 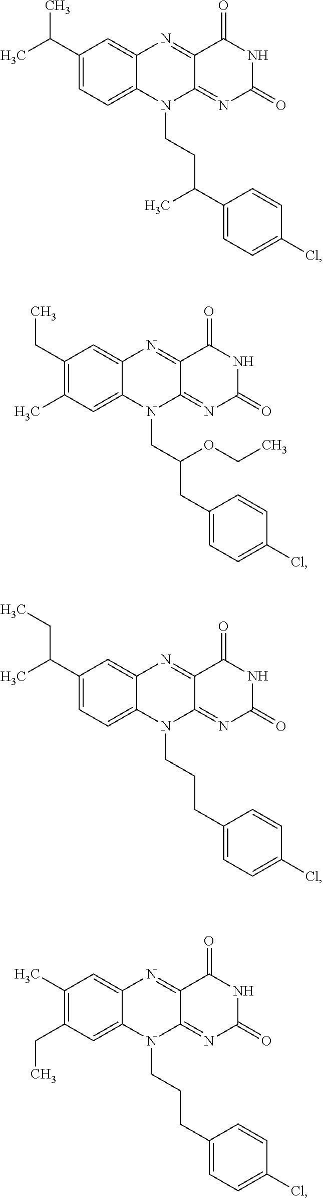 [SMILES: CC(C)c1ccc2c(c1)nc1c(=O)[nH]c(=O)nc-1n2CCC(C)c1ccc(Cl)cc1 ; CCOC(Cc1ccc(Cl)cc1)Cn1c2nc(=O)[nH]c(=O)c-2nc2cc(CC)c(C)cc21 ; CCC(C)c1ccc2c(c1)nc1c(=O)[nH]c(=O)nc-1n2CCCc1ccc(Cl)cc1 ; CCc1cc2c(cc1C)nc1c(=O)[nH]c(=O)nc-1n2CCCc1ccc(Cl)cc1] 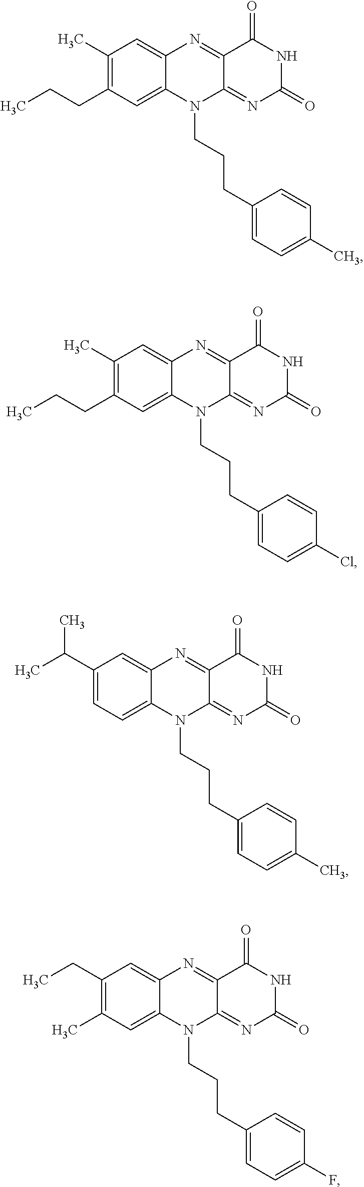 [SMILES: CCCc1cc2c(cc1C)nc1c(=O)[nH]c(=O)nc-1n2CCCc1ccc(C)cc1 ; CCCc1cc2c(cc1C)nc1c(=O)[nH]c(=O)nc-1n2CCCc1ccc(Cl)cc1 ; Cc1ccc(CCCn2c3nc(=O)[nH]c(=O)c-3nc3cc(C(C)C)ccc32)cc1 ; CCc1cc2nc3c(=O)[nH]c(=O)nc-3n(CCCc3ccc(F)cc3)c2cc1C] 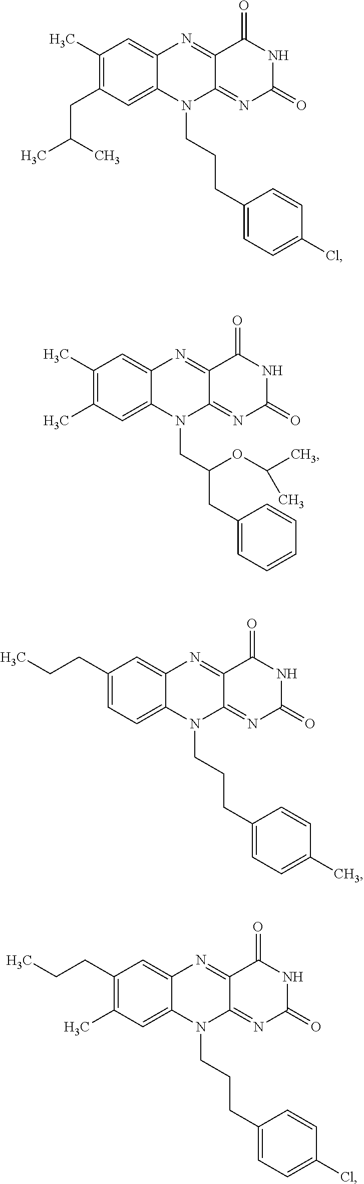 [SMILES: Cc1cc2nc3c(=O)[nH]c(=O)nc-3n(CCCc3ccc(Cl)cc3)c2cc1CC(C)C ; Cc1cc2nc3c(=O)[nH]c(=O)nc-3n(CC(Cc3ccccc3)OC(C)C)c2cc1C ; CCCc1ccc2c(c1)nc1c(=O)[nH]c(=O)nc-1n2CCCc1ccc(C)cc1 ; CCCc1cc2nc3c(=O)[nH]c(=O)nc-3n(CCCc3ccc(Cl)cc3)c2cc1C] 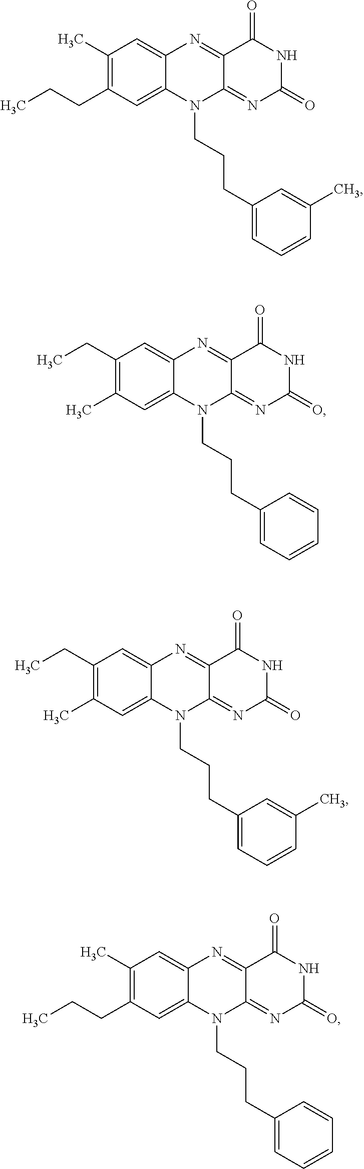 [SMILES: CCCc1cc2c(cc1C)nc1c(=O)[nH]c(=O)nc-1n2CCCc1cccc(C)c1 ; CCc1cc2nc3c(=O)[nH]c(=O)nc-3n(CCCc3ccccc3)c2cc1C ; CCc1cc2nc3c(=O)[nH]c(=O)nc-3n(CCCc3cccc(C)c3)c2cc1C ; CCCc1cc2c(cc1C)nc1c(=O)[nH]c(=O)nc-1n2CCCc1ccccc1] 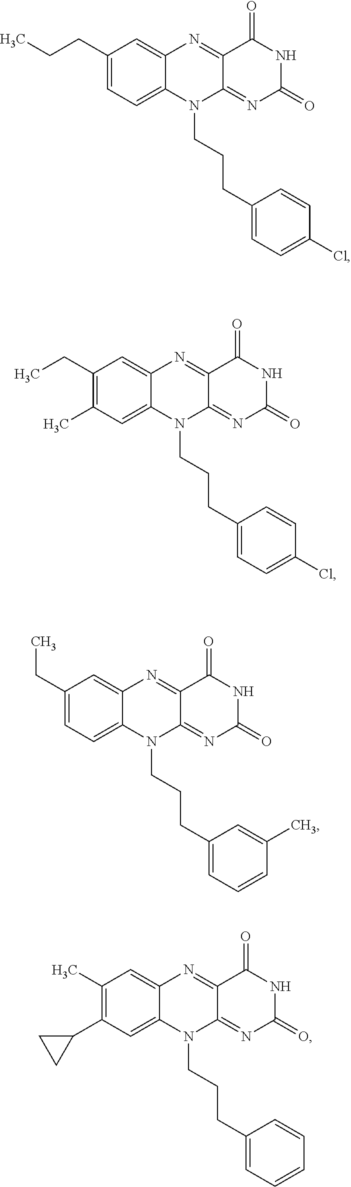 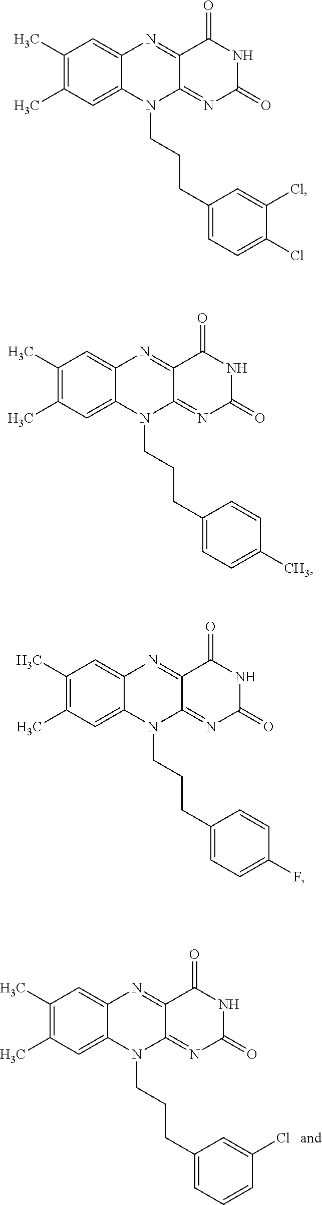 [SMILES: Cc1cc2nc3c(=O)[nH]c(=O)nc-3n(CCCc3ccc(Cl)c(Cl)c3)c2cc1C ; Cc1ccc(CCCn2c3nc(=O)[nH]c(=O)c-3nc3cc(C)c(C)cc32)cc1 ; Cc1cc2nc3c(=O)[nH]c(=O)nc-3n(CCCc3ccc(F)cc3)c2cc1C ; Cc1cc2nc3c(=O)[nH]c(=O)nc-3n(CCCc3cccc(Cl)c3)c2cc1C ; "and"] 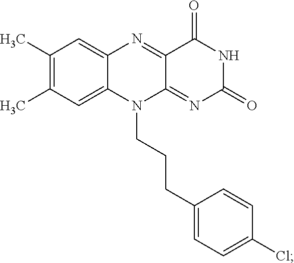 [SMILES: Cc1cc2nc3c(=O)[nH]c(=O)nc-3n(CCCc3ccc(Cl)cc3)c2cc1C]
in free or pharmaceutically acceptable salt form.

24. The method according to any one of claims 19-21, wherein the bacterial infection is a Staphylococcus aureus infection.

25. The method according to claim 24, wherein the compound is selected from any of the following: 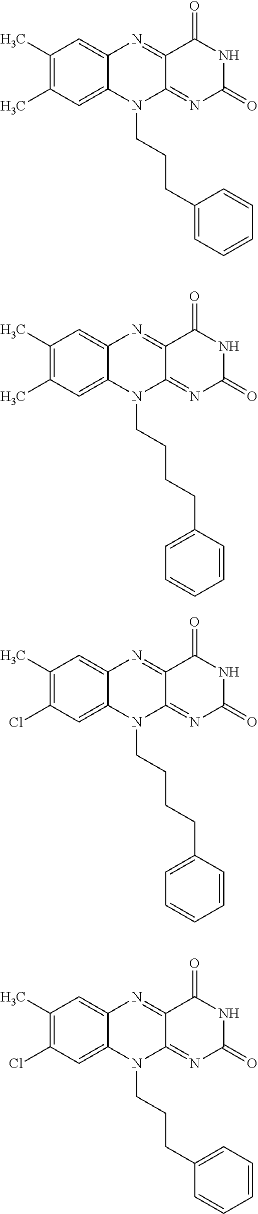 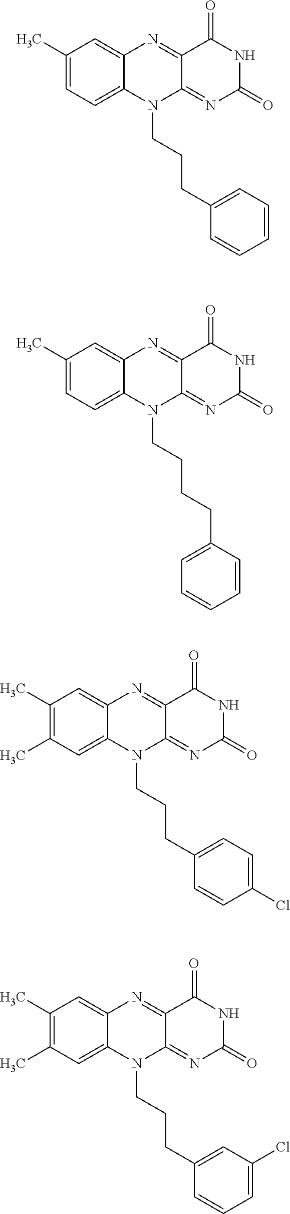 [SMILES: Cc1ccc2c(c1)nc1c(=O)[nH]c(=O)nc-1n2CCCc1ccccc1 ; Cc1ccc2c(c1)nc1c(=O)[nH]c(=O)nc-1n2CCCCc1ccccc1 ; Cc1cc2nc3c(=O)[nH]c(=O)nc-3n(CCCc3ccc(Cl)cc3)c2cc1C ; Cc1cc2nc3c(=O)[nH]c(=O)nc-3n(CCCc3cccc(Cl)c3)c2cc1C] 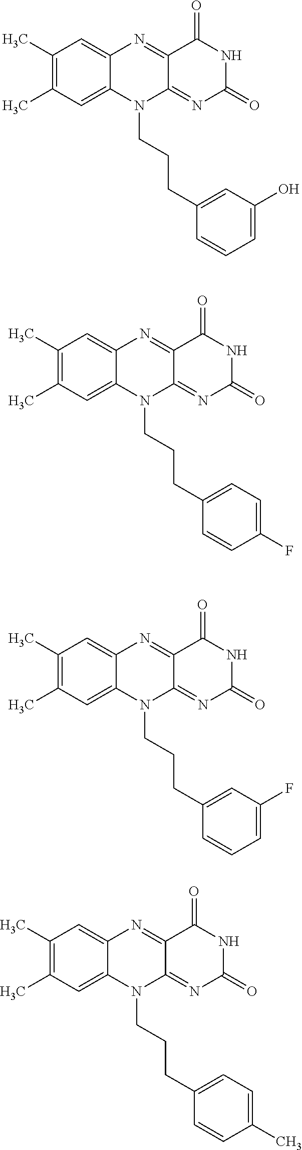 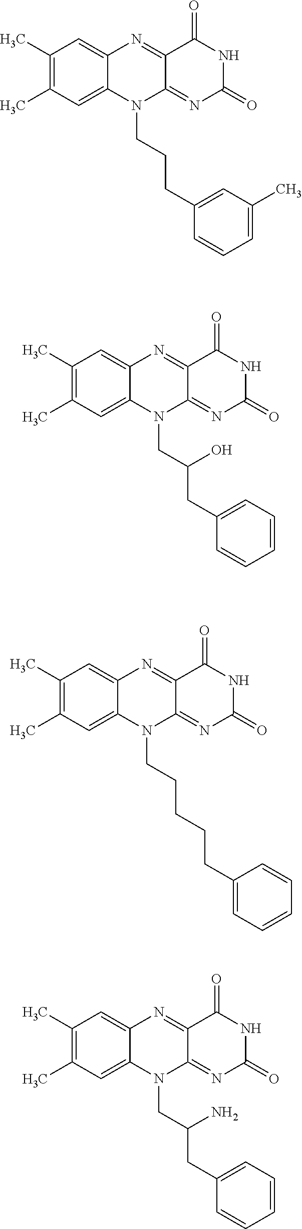 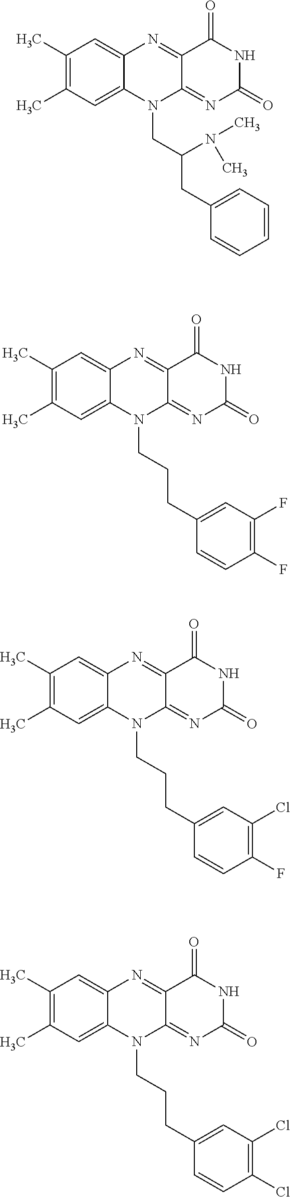 [SMILES: Cc1cc2nc3c(=O)[nH]c(=O)nc-3n(CC(Cc3ccccc3)N(C)C)c2cc1C ; Cc1cc2nc3c(=O)[nH]c(=O)nc-3n(CCCc3ccc(F)c(F)c3)c2cc1C ; Cc1cc2nc3c(=O)[nH]c(=O)nc-3n(CCCc3ccc(F)c(Cl)c3)c2cc1C ; Cc1cc2nc3c(=O)[nH]c(=O)nc-3n(CCCc3ccc(Cl)c(Cl)c3)c2cc1C]
in free or pharmaceutically acceptable salt form.

26. The method according to claim 23 or 25, wherein said infection is by an infectious agent which is resistant to a drug that is not a riboswitch ligand.

27. The method according to claim 25, wherein the infection is an infection which is resistant to one or more drugs selected from a group consisting of a penicillin, vancomycin, cephalosporin and methicillin.

28. The method according to claim 27, wherein the infection is a methicillin-resistant Staphylococcus aureus infection.

29. The method according to claim 23 or 25, wherein the infection is an infection which is resistant to fluoroquinolone (e.g., ciprofloxacin- and/or levofloxacin-resistant infection), metronidazole and/or vancomycin.

30. The method according to any one of claims 19-29, wherein such method is effective for the treatment or prophylaxis of a disease, condition or infection selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD).

31. Use of a compound according to any one of claims 10-18, in the manufacture of a medicament for the treatment or prophylaxis of a bacterial infection as described in any one of claims 19-30.

32. A method for the treatment or prophylaxis of a bacterial infection in a plant comprising administering to said plant an effective amount of a compound of any one of claims 10-18.

33. A pharmaceutical composition according to any one of claims 10-18 for use in the manufacture of a medicament for the treatment or prophylaxis of a bacterial infection as described in any one of claims 19-30.

34. A compound of Formula II″: 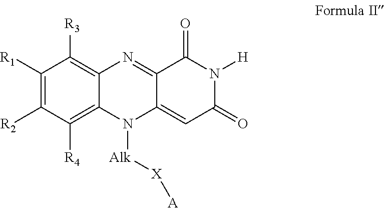
wherein:
(i) Alk is C₁₋₆alkylene (e.g., n-propylene, n-butylene, n-pentylene) optionally substituted with one or more C₁₋₆alkyl (e.g., methyl) or one hydroxy or C₁₋₄alkoxy group;
(ii) X is a single bond, —S— or —O—;
(iii) A is aryl (e.g., phenyl) or heteroaryl (e.g. pyridinyl) optionally substituted with one or more C₁₋₄ alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl), or C₁₋₄alkoxy (e.g., methoxy);
(v) R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C₁₋₄alkoxy)-C₁₋₄alkyl, (2-C₁₋₄ alkoxyethoxy)-C₁₋₄alkyl;
(vi) R₃ is H, C₁₋₄alkyl (e.g., methyl);
(vii) R₄ is H, C₁₋₄alkyl (e.g., methyl);
(viii) Rₐ and R_{b} are independently H, C₁₋₄alkyl (e.g., methyl) or C₃₋₈cycloalkyl (e.g., cyclopropyl, cyclopentyl),
in free or salt form.

35. The compound according to claim 34, wherein said compound is a compound of Formula II: 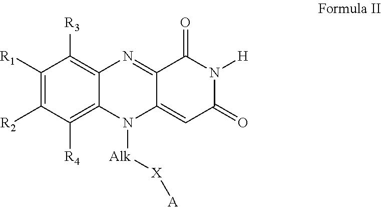
wherein:
(i) Alk is C₁₋₆alkylene (e.g., n-propylene, n-butylene, n-pentylene) optionally substituted with one hydroxy or C₁₋₄alkoxy group;
(ii) X is a single bond, —S— or —O—;
(iii) A is aryl (e.g., phenyl) or heteroaryl (e.g. pyridinyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
(iv) R₁ is H, C₁₋₄alkyl (e.g., methyl), or C₁₋₄alkoxy (e.g., methoxy);
(v) R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C₁₋₄ alkoxy)-C₁₋₄alkyl, (2-C₁₋₄alkoxyethoxy)-C₁₋₄alkyl;
(vi) R₃ is H, C₁₋₄alkyl (e.g., methyl);
(vii) R₄ is H, C₁₋₄alkyl (e.g., methyl);
(viii) Rₐ and R_{b} are independently H, C₁₋₄alkyl (e.g., methyl) or C₃₋₈cycloalkyl (e.g., cyclopropyl, cyclopentyl),
in free or salt form.

36. The compound according to claim 34 or 35, wherein X is a single bond, wherein said compound is a compound of Formula II′: 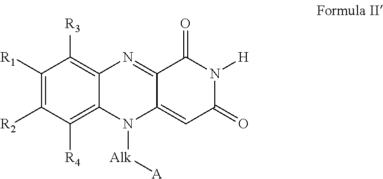
wherein:
(i) Alk is C₁₋₆alkylene (e.g., n-propylene, n-butylene, n-pentylene) optionally substituted with one hydroxy or C₁₋₄alkoxy group;
(ii) A is aryl (e.g., phenyl) or heteroaryl (e.g. pyridinyl) optionally substituted with one or more C₁₋₄alkyl (e.g., methyl), C₁₋₄alkoxy (e.g., methoxy), hydroxy, halo (e.g., Cl, F), haloC₁₋₄alkyl (e.g., CF₃), —O-haloC₁₋₄alkyl (e.g., —OCF₃);
(iii) R₁ is H, C₁₋₄alkyl (e.g., methyl), or C₁₋₄alkoxy (e.g., methoxy);
(iv) R₂ is H, C₁₋₄alkyl (e.g., methyl), C₁₋₄alkoxy (e.g., methoxy), halo (e.g., Cl), C₃₋₈cycloalkyl-C₁₋₄alkyl, —C₁₋₄alkyl-N(Rₐ)(R_{b}), (C₁₋₄alkoxy)-C₁₋₄alkyl, (2-C₁₋₄alkoxyethoxy)-C₁₋₄alkyl;
(v) R₃ is H, C₁₋₄alkyl (e.g., methyl);
(vi) R₄ is H, C₁₋₄alkyl (e.g., methyl)
(vii) Rₐ and R_{b} are independently H, C₁₋₄alkyl (e.g., methyl) or C₃₋₈cycloalkyl (e.g., cyclopropyl, cyclopentyl)
in free or salt form.

37. A pharmaceutical composition comprising a compound according to claim 34, 35 or 36, in free or pharmaceutically acceptable salt form, in admixture with a pharmaceutically acceptable diluent or carrier.

38. A method for the treatment or prophylaxis of a bacterial infection comprising administering to a patient in need of such treatment an effective amount of a compound according to claim 34, 35 or 36, in free or pharmaceutically acceptable salt form.

39. The method according to claim 38, wherein the infection is a Gram-positive or Gram-negative bacterial infection.

40. The method according to any one of claims 38-39, wherein the bacterial infection is selected from a group consisting of Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and Borrelia burgdorferi.

41. The method according to any one of claim 40, wherein the bacterial infection is a C. difficile infection.

42. The method according to any one of claims 38-41, wherein said infection is by an infectious agent which is resistant to a drug that is not a riboswitch ligand.

43. The method according to claim 42, wherein the infection is an infection which is resistant to fluoroquinolone (e.g., ciprofloxacin- and/or levofloxacin-resistant infection), metronidazole and/or vancomycin.

44. The method according to any one of claims 38-43, wherein such method is effective for the treatment or prophylaxis of a disease, condition or infection selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD).

45. Use of a compound according to claim 34, 35 or 36 in free or pharmaceutically acceptable salt form, in the manufacture of a medicament for the treatment or prophylaxis of a bacterial infection.

46. Use according to claim 45, wherein the bacterial infection is selected from a group consisting of Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and Borrelia burgdorferi.

47. Use of a compound according to claim 34, 35 or 36 in free or pharmaceutically acceptable salt form, in the manufacture of a medicament for the treatment of a disease, condition or infection selected from a group consisting of anthrax, staphylococcal scalded skin syndrome (staph infections), pneumonia, impetigo, boils, cellulitis, folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, brain abscess, tularemia, urinary tract infection, empyema, food poisoning, diarrhea, conjunctivitis and clostridium difficile associated disease (CDAD).

48. A method for the treatment or prophylaxis of a bacterial infection in a plant comprising administering to said plant an effective amount of a compound according to claim 34, 35 or 36 in free or salt form.

49. A pharmaceutical composition according to claim 37 for use in the manufacture of a medicament for the treatment or prophylaxis of a bacterial infection.

50. The pharmaceutical composition according to claim 49, wherein the bacterial infection is selected from a group consisting of Clostridium difficile, Staphylococcus epidermidis, Staphylococcus aureus, Streptococcus pneumoniae, Pseudomonas aeruginosa, Acinetobacter baumannii, Escherichia coli, Haemophilus influenzae, Enterococcus faecalis, Streptococcus pyogenes, Listeria monocytogenes, Salmonella enterica, Vibrio cholerae, Brucella melitensis, Bacillus anthracis, Francisella tularensis, Moraxella catarrhalis, Klebsiella pneumoniae, Yersinia pestis, Streptococcus viridans, Enterococcus faecium, and Borrelia burgdorferi.
